(19) United States
(12) Patent Application Publication
(10) Pub. No.: US 2024/0269139 A1
(43) Pub. Date: Aug. 15, 2024

(54) COMPOUNDS FOR TARGETED PROTEIN DEGRADATION OF KINASES

(71) Applicants: DANA-FARBER CANCER INSTITUTE, INC., Boston, MA (US); Korea Institute of Science and Technology (KIST), Seoul (KR)

(72) Inventors: Nathanael S. Gray, Boston, MA (US); Eric S. Fischer, Chestnut Hill, MA (US); Fleur M. Ferguson, Cambridge, MA (US); Katherine Donovan, Brookline, MA (US); Jonathan W. Bushman, Boston, MA (US); Taebo Sim, Seoul (KR); Debabrata Bhunia, Hyderabad (IN); SeongShick Ryu, Seoul (KR)

(73) Assignees: DANA-FARBER CANCER INSTITUTE, INC., Boston, MA (US); Korea Institute of Science and Technology (KIST), Seoul (KR)

(21) Appl. No.: 18/033,872

(22) PCT Filed: Oct. 26, 2021
(86) PCT No.: PCT/US2021/056545

§ 371 Date: Apr. 26, 2023

Related U.S. Application Data
(60) Provisional application No. 63/105,728, filed on Oct. 26, 2020.

(51) Int. Cl. A61K 31/506; A61K 31/4725; A61K 31/5025; A61K 31/519; A61K 31/55; G01N 33/50

(52) U.S. Cl. CPC A61K 31/506; A61K 31/4725; A61K 31/5025; A61K 31/519; A61K 31/55; G01N 33/502; G01N 2333/912; G01N 2560/00; G01N 2570/00

(57) ABSTRACT

The present invention relates to bifunctional compounds for targeted kinase degradation, methods for treating diseases or conditions mediated by aberrant kinase activity, and methods for identifying degradable kinases and optimal kinase:scaffold pairs.

### RELATED APPLICATIONS

This application is a national stage application, filed under 35 U.S.C. § 371, of International Application No. PCT/US2021/056545, filed Oct. 26, 2021, which claims the benefit of priority under 35 U.S.C. § 119(e) to U.S. Provisional Application No: 63/105,728, filed on Oct. 26, 2020, each of which is incorporated herein by reference in its entirety.

### GOVERNMENT LICENSE RIGHTS

This invention was made with government support under grant numbers R01CA214608. R01CA218278, and U24-DKI16204 awarded by the National Institutes of Health. The government has certain rights in the invention.

### BACKGROUND OF THE INVENTION

Targeted protein degradation refers to the use of small molecules to induce ubiquitin-dependent degradation of proteins. These degrader molecules are of great interest in drug development as they can address previously inaccessible targets (Russ and Lampel, Drug Discov Today 10(2377):1607-1610 (2005). However, degrader development remains an inefficient and empirical process due to a lack of understanding of the key properties that require optimization (Kostic and Jones. Trends Pharmacol Sci. 41(5):305-317 (2020)).

### SUMMARY OF THE INVENTION

A first aspect of the present invention is directed to bifunctional compounds (also referred to as degraders) and pharmaceutically acceptable salts and stereoisomers thereof for targeted degradation of kinases.

Another aspect of the present invention is directed to a pharmaceutical composition containing a therapeutically effective amount of a bifunctional compound of the present invention or a pharmaceutically acceptable salt or stereoisomer thereof, and a pharmaceutically acceptable carrier.

In another aspect of the present invention, methods of making the bifunctional compounds are provided.

Another aspect of the present invention is directed to a method of treating a disease or disorder associated with aberrant activity of AP2-associated protein kinase 1 (AAK1), ABL proto-oncogene (ABL)1, ABL2, Serine/Threonine kinase (AKT)2, AKT3, Aurora kinase (AURK)4, AURKA, AURKB, branched chain ketoacid dehydrogenase kinase (BCKDK). B-lymphoid tyrosine kinase (BLK), BMP-2-inducible protein kinase (BMP2K), Bone morphogenetic protein receptor type-1A (BMPR1A), mitotic checkpoint serine/threonine-protein kinase BUB 1 (BUB1), BUB1B, calcium/calmodulin-dependent protein kinase kinase 1 (CAMKK1), cell division cycle 7 (CDC7), cyclin-dependent kinase (CDK)1, CDK10, CDK11A, CDK11B, CDK12, CDK13, CDK14, CDK16, CDK17, CDK18, CDK2, CDK3, CDK4, CDK5, CDK6, CDK7, CDK9. Checkpoint kinase 1(CHEK1), citron Rho-interacting kinase (CIT), CDC Like Kinase 1 (CLK1), coenzyme Q8 (COQ8)A, COQ8B, Tyrosine-protein kinase CSK (CSK), casein kinase 1 (CSNK1)A1, CSNK1D, CSNK1E, death-associated protein kinase 1 (DAPK1), discoidin domain-containing receptor 2 (DDR2), eukaryotic translation initiation factor 2-alpha kinase (EIF2AK)2, EIF2AK4, ephrin type-A receptor (EPHA)1, EPHA2, EPHA3, ephrin type-B receptor (EPHB)2, EPHB3, EPHB4, EPHB6, endoplasmic reticulum to nucleus signaling 1 (ERN1), tyrosine-protein kinase Fer (FER), fibroblast growth factor receptor 1 (FGFR1), fibroblast growth factor receptor 2(FGR2), proto-oncogene tyrosine-protein kinase Fyn (FYN), cyclin G-associated kinase (GAK), glycogen synthase kinase 3 (GSK3)A, GSK3B, homeodomain-interacting protein kinase 1 (HIPK1), interleukin-1 receptor-associated kinase (IRAK)1. IRAK4, tyrosine-protein kinase ITKITSK (ITK), large tumor suppressor kinase 1 (LATS1), lymphocyte cell-specific protein-tyrosine kinase (LCK), LIM domain kinase (LIMK)1, LIMK2, leucine-rich repeat kinase 2 (LRRK2), tyrosine-protein kinase Lyn (LYN), dual specificity mitogen-activated protein kinase kinase 5 (MAP2K5), mitogen-activated protein kinase kinase kinase (MAP3K)1, MAP3K11, MAP3K12, MAP3K20, MAP3K21, MAP3K7, mitogen-activated protein kinase kinase kinase kinase (MAP4K)l, MAP4K2, MAP4K3, MAP4K5, mitogen-activated protein kinase (MAPK)1L, MAPK12, MAPK14, MAPK6, MAPK7, MAPK8, MAPK9, mitogen-activated protein kinase-activated protein kinase (MAPKAPK)2, MAPKAPK3, MAPKAPK5, microtubule affinity regulating kinase (MARK)2, MARK3, MARK4, microtubule-associated serine/threonine-protein kinase 3 (MAST3), maternal embryonic leucine zipper kinase (MELK), misshapen like kinase 1 (MINK1), MAP kinase-interacting serine/threonine-protein kinase 2 (MKNK2), never in mitosis A-related kinase (NEK)2. NEK9, nemo like kinase (NLK). NUAK family SNF1-like kinase 1 (NUAK1), serine/threonine-protein kinase PAK 4 (PAK4), serine/threonine-protein kinase PDIK1L (PDIK1L), 3-phosphoinositide-dependent protein kinase (PDK)1, PDK2, PDK3, phosphatidylinositol 4,5-bisphosphate 3-kinase catalytic subunit gamma isoform (PIK3CG), serine/threonine-protein kinase pim-2 (PIM2), membrane-associated tyrosine- and threonine-specific cdc2-inhibitory kinase (PKMYT1), serine/threonine-protein kinase N3 (PKN3), polo like kinase (PLK)1, PLK4, PEAK1 related, kinase-activating pseudokinase 1 (PRAG1), 5′-AMP-activated protein kinase catalytic subunit alpha (PRKAA)1. PRKAA2, protein tyrosine kinase (PTK)2, PTK2B, PTK6, RIO kinase 2 (RIOK2), receptor-interacting serine/threonine-protein kinase (RIPK)1. RIPK2, ribosomal protein S6 kinase 2 alpha (RPS6KA)1, RPS6KA3, RPS6KA4, RPS6KA6, ribosomal protein S6 kinase beta 1 (RPS6KB1), ribosomal protein S6 kinase beta C1 (RPS6KC1), SH3 domain binding kinase 1 (SBK1), serum/glucocorticoid-regulated kinase 3 (SGK3), salt inducible kinase (SIK)2, SIK3, SIKA2, sucrose nonfermenting 1-related kinase (SNRK), proto-oncogene tyrosine-protein kinase Src (SRC), serine/threonine-protein kinase (STK)10, STK17A, STK17B, STK32C, STK33, STK35, STK38, STK4, STK40, thousand and one amino-acid kinase (TAOK)2, TAOK3, tyrosine-protein kinase Tec (TEC), dual specificity testis-specific protein kinase 2 (TESK2), transforming growth factor beta receptor 1 (TGFBR1), tyrosine kinase non receptor (TNK)1, TNK2, Tribbles homolog 3 (TRIB3), transient receptor potential cation channel subfamily M member 7 (TRPM7), dual specificity protein kinase TTK (TTK), non-receptor tyrosine-protein kinase (TYK2) TYK2, U2AF homology motif kinase 1 (UHMK1), unc-51 like autophagy activating kinase (ULK)1, ULK3, WEE1 G2 checkpoint kinase (WEE1), or YES proto-oncogene 1 (YES1), that includes administering a therapeutically effective amount of a bifunctional compound of the present invention or a pharmaceutically acceptable salt or stereoisomer thereof, to a subject in need thereof.

Accordingly, the bifunctional compounds of the present invention may serve as a set of new chemical tools for AAK1, ABL1, ABL2, AKT2, AKT3, AURK4, AURKA, AURKB, BCKDK, BLK, BMP2K. BMPR1A, BUB1, BUB1B, CAMKK1, CDC7, CDK1, CDK10, CDK11A, CDK11B, CDK12, CDK13, CDK14, CDK16, CDK17, CDK18, CDK2, CDK3, CDK4, CDK5, CDK6, CDK7, CDK9, CHEK1, CIT, CLK1, COQ8A, COQ8B, CSK, CSNK1A1, CSNK1D, CSNK1E, DAPK1, DDR2, EIF2AK2, EIF2AK4, EPHA1, EPHA2, EPHA3, EPHB2, EPHB3, EPHB4, EPHB6, ERN1, FER, FGFR1, FGR2, FYN, GAK, GSK3A, GSK3B, HIPK1, IRAK1, IRAK4, ITK, LATS1, LCK, LIMK1, LIMK2, LRRK2, LYN, MAP2K5, MAP3K1, MAP3K11, MAP3K12. MAP3K20, MAP3K21, MAP3K7, MAP4K1, MAP4K2, MAP4K3, MAP4K5, MAPK11, MAPK12, MAPK14, MAPK6, MAPK7, MAPK8, MAPK9, MAPKAPK2, MAPKAPK3, MAPKAPK5, MARK2, MARK3, MARK4, MAST3, MELK, MINK1, MKNK2, NEK2, NEK9, NLK, NUAK1, PAK4, PDIK1L, PDK1, PDK2, PDK3, PIK3CG, PIM2. PKMYT1, PKN3, PLK1, PLK4, PRAG1, PRKAA1, PRKAA2. PTK2 PTK2B, PTK6, RIOK2, RIPK1, RIPK2, RPS6KAL, RPS6KA3, RPS6KA4, RPS6KA6, RPS6KB1, RPS6KC1, SBK1, SGK3, SIK2, SIK3, SIKA2, SNRK, SRC, STK10, STK17A, STK17B, STK32C, STK33, STK35, STK38, STK4, STK40, TAOK2, TAOK3, TEC, TESK2, TGFBR1, TNK1, TNK2, TRIB3, TRPM7, TTK, TYK2, UHMK1, ULK1, ULK3, WEE1, and YES1 knockdown, exemplify a broadly applicable approach to arrive at degraders that may provide effective treatments for diseases and disorders associated with a kinase selected from AAK1, ABL1, ABL2, AKT2, AKT3, AURK4, AURKA, AURKB. BCKDK, BLK, BMP2K, BMPR1A, BUB1, BUB1B. CAMKK1, CDC7, CDK1, CDK10, CDK11A, CDK11B, CDK12, CDK13, CDK14, CDK16, CDK17, CDK18, CDK2, CDK3, CDK4, CDK5, CDK6, CDK7, CDK9, CHEK1, CIT. CLK1, COQ8A, COQ8B, CSK, CSNK1A1, CSNK1D, CSNK1E, DAPK1, DDR2, EIF2AK2, EIF2AK4. EPHA1, EPHA2, EPHA3. EPHB2, EPHB3, EPHB4, EPHB6, ERN1. FER, FGFR1, FGR2, FYN, GAK, GSK3A, GSK3B, HIPK1, IRAK1, IRAK4, ITK, LATS1, LCK, LIMK1, LIMK2, LRRK2, LYN, MAP2K5, MAP3K1, MAP3K11, MAP3K12, MAP3K20, MAP3K21, MAP3K7, MAP4K1, MAP4K2, MAP4K3, MAP4K5, MAPK11, MAPK12, MAPK14, MAPK6, MAPK7, MAPK8, MAPK9, MAPKAPK2, MAPKAPK3, MAPKAPK5, MARK2, MARK3, MARK4, MAST3, MELK, MINK1, MKNK2, NEK2, NEK9, NLK, NUAK1, PAK4, PDIK1L, PDK1, PDK2, PDK3, PIK3CG, PIM2, PKMYT1. PKN3, PLK1, PLK4, PRAG1, PRKAA1, PRKAA2, PTK2, PTK2B, PTK6. RIOK2, RIPK1, RIPK2, RPS6KA 1, RPS6KA3, RPS6KA4, RPS6KA6, RPS6KB1. RPS6KC1. SBK1, SGK3, SIK2, SIK3, SIKA2, SNRK, SRC, STK10, STK17A, STK17B, STK32C, STK33, STK35, STK38, STK4, STK40, TAOK2, TAOK3, TEC, TESK2, TGFBR1. TNK1, TNK2, TRIB3, TRPM7, TTK, TYK2. UHMK1, ULK1, ULK3, WEE1, and YES1, e.g., cancer, neurodegenerative diseases, inflammatory disorders, infectious diseases, and autoimmune diseases.

In some aspects, the bifunctional compounds of the present invention may be useful tools for rapidly interrogating targeted protein degradation of a plurality of kinases.

A further aspect of the present invention is directed to methods for a degradable kinase comprising:
assembling a kinase-targeting degrader library comprising a plurality of kinase-targeting scaffolds:
prescreening candidate degrader compounds for cellular permeability in a relevant E3-ligase target engagement assay;
selecting a cell permeable degrader for further characterization of degradation targets; treating a cell with the selected cell permeable degrader:
employing whole cell multiplexed quantitative proteomics to measure changes in abundance of the proteome in response to treatment with the degrader relative to DMSO; and analyzing the generated datasets to calculate kinase degradation frequency across the library, as a measure of target tractability.

In some embodiments, the methods may also be used for rapidly identifying optimal kinase:scaffold pairs.

In some embodiments, the degradation targets are further characterized using unbiased mass-spectrometry-based global proteomics analysis, based on chemical diversity and ranking in cellular ligase engagement assays relative to close analogs.

Chemo-proteomics was used to annotate the ‘degradable kinome’. The comprehensive dataset provided chemical leads for approximately 200 kinases and demonstrated that the current practice of starting from the highest potency binder is an inefficient method for discovering leads. The dataset also enabled rapid chemical probe discovery for ‘understudied kinases’. Highly multi-targeted degraders were developed to answer fundamental questions about the ubiquitin proteasome system, such as the role of the p97 unfoldase targeted protein degradation. The methods of the present invention may not only fuel kinase degrader discovery, but also provide a blueprint for evaluating targeted degradation across entire gene families, to accelerate understanding of targeted protein degradation beyond the kinome.

### BRIEF DESCRIPTION OF THE DRAWINGS

FIG. 1A-FIG. 1J are a series of schematics, graphs, and a heatmap showing an experimental map of the degradable kinome. FIG. 1A is schematic representing mode of action of targeted protein degraders. FIG. 1B is workflow detailing the experimental approach taken in this study. FIG. 1C graph of the features of the profiled chemical library of protein kinase targeting heterobifunctional degrader molecules. Chemical structures reported in Table 1. FIG. 1D is a kinome tree presenting protein kinases that were significantly downregulated by at least one degrader. Image created using KinMap, illustration reproduced courtesy of Cell Signaling Technology®, Inc. FIG. 1E is graph showing proportion of the human protein kinome detected and degraded by whole cell quantitative proteomics analysis in at least one experiment described herein. Data reported in Tables 1-2. FIG. 1F is a graph showing a comparison of degraded kinase targets reported in the literature and in this study. Literature searching was performed in PubMed, using search terms ‘kinase PROTAC1@’ and ‘kinase degrader’. FIG. 1(G is a graph showing the number of independent compound treatments for which degradation was observed for each kinase. Inset, the top 20 most frequently degraded kinases. FIG. 1H is a heatmap correlation comparison of kinase degradability score with PubMed Count and Protein Data Bank (PDB) count knowledge metrics. FIG. 1I is a table showing proportion of understudied kinases, lipid kinases and pseudokinases detected and degraded by whole cell quantitative proteomics analysis in at least one experiment described herein. FIG. 1J id a scatterplot depicting relative protein abundance following treatment of MOLT-4 cells with 1 μM DD-03-156 for 5 h compared to DMSO treatment. Inset, chemical structure of DD-03-156. Scatterplot displays fold change in abundance relative to DMSO.

FIG. 2A-FIG. 2G are a set of plots, heatmap, immunoblots, and a graph showing that the degradable kinome dataset accelerates lead discovery. FIG. 2A is a heatmap comparing relative fold change in protein abundance in response to treatment with indicated degrader. Inset, chemical structure of dasatinib-based CSK degrader DB-3-291. FIG. 2B is scatterplot depicting relative protein abundance following treatment of MOLT-4 cells with 1 μM DB-3-291 for 5 h compared to DMSO treatment. Scatterplot displays fold change abundance relative to DMSO. FIG. 2C is a kinome tree representing the kinase degradability (DK) score (number of times kinase is degraded by a unique degrader) calculated for each of the protein kinases degraded, illustrating the high calculated degradability of AURKA. Image created using KinMap, illustration reproduced courtesy of Cell Signaling Technology®, Inc. FIG. 2D is a scheme showing a strategy for conversion of Alisertib into selective AURKA degrader dAURK-4. FIG. 2E is a scatterplot depicting relative protein abundance following treatment of MOLT-4 cells with 1 μM dAURK-4 for 5 h compared to DMSO treatment. Scatterplot displays fold change in abundance relative to DMSO. FIG. 2F is a picture of an immunoblot analysis of MM.1S cells treated with the indicated concentration of dAURK-4 for 4 or 24 h. Data in FIG. 2F are representative of n=2 independent experiments. FIG. 2G is graph showing DMSO-normalized antiproliferation of MM.1S cells treated with Alisertib or dAURK-4. Data are presented as mean±s.d. of n=3 biologically independent samples and are representative of n=2 independent experiments.

FIG. 3A-FIG. 3F are a series of schematics, chemical structures, and scatterplots showing cellular target engagement does not predict degradation FIG. 3A is a schematic representation of multiplexed tandem mass tag (TMT)-based quantitative proteomics workflow used herein. FIG. 3B is a Schematic representation of activity-based protein profiling (ABPP)-based KiNativ™ proteomics workflow used for target engagement measurements. FIG. 3C is a schematic representation of AP-MS approach used to enrich for degrader-mediated ternary complexes with cereblon (CRBN). FIG. 3D depicts the chemical structures of the 4 multitargeted degrader probes. FIG. 3E is a scatterplot comparing kinase engagement with kinase degradation. Plot shows the % inhibition of ABPP probe binding observed for each kinase (x-axis) in a KiNativ™ experiment. KiNativ™ data are from n=2 technically independent samples, proteomics analysis data are from n=1 biologically independent treatment samples. Negative KiNativ™ values were interpreted as 0% inhibition of binding. FIG. 3F is a bar chart showing the proportion of degraded kinase targets for which detectable target engagement (TE, >35% inhibition of binding) and degradation (FC>1.25, P-value<0.01) were observed for the 4 compounds tested.

FIG. 4A-FIG. 4F are a series of plots and graphs depicting effects of temary complex formation and target protein abundance on degrader efficacy. FIG. 4A: Left. Protein abundance following treatment of HEK293T cells treated with 1 μM of the indicated compound for 5 h compared to DMSO treatment. Scatterplots depict fold change in abundance relative to DMSO. Right. Rank order plot showing the ranked relative abundance ratios of enriched proteins in FLAG-CRBN AP-MS experiments from HEK293T cells co-treated with proteasome inhibitor and 1 μM of the indicated compound for 5 h compared to co-treated with proteasome inhibitor and DMSO control. Data in scatterplots are from n=2 biologically independent treatment samples. Data in rank order plots are from n=3 biologically independent treatment samples. FIG. 4B is a bar chart depicting the proportion of targets complexed and degraded by the indicated compounds. FIG. 4C is a set of Venn diagrams showing unique and overlapping kinase hits found for each compound in MOLT-4 (blue), KELLY (orange) and HEK293T (gray) cells. FIG. 4D is graph showing a kinome wide comparison of the degradation frequency and the relative protein abundance in MOLT-4 cells. FIG. 4E is a bar plot showing the average relative expression of CRL4CRBN degradation machinery proteins (left) and number of kinases degraded by each of the indicated degraders in MOLT-4, KELLY and HEK293T cells (right). Protein expression measurements were made using whole cell quantitative proteomics to measure protein abundances across the three indicated cell lines. Average abundance measurements were derived from n=2 independent biological treatments. FIG. 4F is a plot showing correlation of kinase degradability score and reported protein half-life in listed cell types.

FIG. 5A-FIG. 5D are a series of plots and a diagram showing that varying the target recruiting ligase can influence degrader selectivity. FIG. 5A-FIG. 5C are a set of chemical structures and a scatterplots showing the log 2 FC pairwise comparison of relative protein abundance resulting from treatment with Von Hippel-Lindau tumor suppressor (VHL) vs CRBN degrader pairs, 5D is a Venn diagram illustrating the target overlap for the aggregated data in FIG. 5A-FIG. 5C.

FIG. 6A-FIG. 6E are a series chemical structures, graphs, and heatmaps showing that protein kinases have varied tolerance for subtle changes in linker design. FIG. 6A is a series of evaluated chemical structures. FIG. 6B is a series of graphs of intracellular ligase engagement assay for indicated compounds. BRD4^{BD2}-GFP reporter cells were treated with increasing concentration of indicated compound for 5 hours in the presence of dBET6. Relative abundance of BRD4^{BD2}-GFP was measured by fluorescence-activated cell sorting (FACS). Data are represented as means±s.d of three replicates (n=3). FIG. 6C is a heatmap showing log 2 FC of kinases determined to be hits (FC>1.25 and P-value<0.01) following a 5 h treatment of MOLT-4 cells with 0.1 μM of the indicated compounds. FIG. 6D is a heatmap plotting log 2 FC of known immunomodulatory imide drug (IMiD) off-targets (determined to be hits (FC>1.25 and P-value<0.01) following a 5 h treatment of MOLT-4 cells with 0.1 μM of the indicated compounds. FIG. 6E is a split bar plot showing the number of CRBN-recruiting degraders found to hit at least one known IMiD off-target compared to the number that do not hit IMiD off-targets. CRBN-recruiting degraders are categorized according their linker attachment chemistry.

FIG. 7A-FIG. 7D are a series of scatterplots, chemical structures, and a graph showing that proteasomal degradation of most kinases is p97 dependent. FIG. 7A is a series of scatterplots depicting the fold change in relative abundance following a 5-hour treatment of MOLT-4 cells with 1 μM of the indicated compounds with (blue) and without (orange) co-treatment with 5 μM of CB-5083, a p97 inhibitor, and compared to DMSO control. FIG. 7B is a bar chart comparing the relative protein abundance of the top 5 degraded kinases from each of the indicated treatments in FIG. 7A. Bars indicate relative protein expression in response to inhibition of p97, with 5 μM of CB-5083, over a time course experiment in MOLT-4. Relative expression data are represented as mean±s.d. of from n=2 biologically independent treatment. FIG. 7C is a series of chemical structures of GNF7-based kinase degraders utilizing either CRBN, VHL, or (inhibitors of apoptosis protein) TAP binding moiety. FIG. 7D is a series of scatterplots depicting the fold change in relative abundance following a 5-hour treatment of MOLT-4 cells with 1 μM of the indicated compounds with (blue) and without (orange) co-treatment with 5 μM of CB-5083, a p97 inhibitor, and compared to DMSO control.

FIG. 8A-FIG. 8B are a series of scatterplots depicting kinase hits across degradable kinome dataset. The scatterplots in FIG. 8A-FIG. 8B depict the fold change in relative abundance comparing treatment to DMSO control determined using quantitative proteomics.

FIG. 9A-FIG. 9E are a series of graphs and a heatmap showing proteomics hit generation and analysis of kinase transcript levels. FIG. 9A is a pie chart depicting the proportion of kinases unique to the extended kinome detected in at least one experiment and degraded in at least one compound treatment in this study. FIG. 9B is a heatmap comparing relative abundance of representative kinase transcripts following treatment with DMSO or 1 μM SK-3-91 for the indicated time periods. FIG. 9C is plot of mean reads per gene observed by RNA-sequencing analysis of MOLT-4 cells treated with 1 μM SK-3-91 or DMSO for the indicated time periods. Data in FIG. 9B and FIG. 9C are from n=4 biologically independent samples. FIG. 9D is a plot showing full correlation relationships between kinase degradation frequency, maximum fold change in protein abundance and common knowledge metrics (PDB and PubMed count). FIG. 9E is a plot showing correlation between degradation frequency and common knowledge metrics (PDB and PubMed count) of how well studied a gene of interest is.

FIG. 10A-FIG. 10F are a series of graphs and scatterplots showing an assessment of the relationship between cellular target engagement and degradation. FIG. 10A is a plot of various 4-degrader combinations and the number of unique protein kinases that can be degraded by that combination. FIG. 10B is a series of graphs of intracellular ligase engagement assay for indicated compounds. BRD4^{BD2}-GFP reporter cells were treated with increasing concentration of lenalidomide or indicated compound for 5 hours in the presence of dBET6. Relative abundance of BRD4^{BD2}-GFP was measured by FACS. Data are represented as means±s.d. of three replicates (n=3). FIG. 10C is a series of dendrograms of kinase inhibition of MOLT-4 CRBN^{-/-} cells treated with 1 μM of indicated multi-kinase targeting degraders for 5 hours. FIG. 10D is a series of scatterplots depicting the fold change in relative abundance comparing treatment 1 μM SK-3-91, DB0646, SB1-G-187, or WH-10417-099 to DMSO control for 5 hours in MOLT-4 cells determined using quantitative proteomics. Log₂ FC is displayed on the y-axis and negative log₁₀ P value on the x-axis. FIG. 10E is a scatterplot comparing the c Log P of degrader molecules and the number of kinase degradation targets. c Log P was calculated using Collaborative Drug Discovery (CDD) Vault. FIG. 10F is a bar graph showing the relative transcript levels of selected kinases after treatment with DMSO or SK-3-91 for 1 hour and for 4 hours. Graph depicts replicates presented as means±s.d. (n=4).

FIG. 11A-FIG. 11F are a series of plots, heatmaps, and a table showing an assessment of the impact of temary complex formation and protein expression on protein degradation. FIG. 11A is rank order plot showing the ranked relative abundance ratios of enriched proteins in FLAG-CRBN AP-MS experiments from HEK293T cells co-treated with proteasome inhibitor and 1 μM of Pomalidomide for 5 h. Data are from n=2 biologically independent samples. FIG. 11B is a heatmap comparing the relative fold change in protein abundance of protein kinases enriched by the presence of indicated degraders in AP-MS experiments relative to DMSO control. FIG. 11C is a table summarizing the number of protein kinases quantified and degraded in response to each of the indicated compounds (1 μM, 5 h) in MOLT-4, KELLY and HEK293T cells. FIG. 11D is a kinome wide comparison of the fold change in relative abundance and the relative protein abundance of protein kinases in MOLT-4, KELLY and HEK293T cells. FIG. 11E is a set of heatmaps displaying the log 2 FC in protein abundance resulting from MOTL-4 cells treated in a time course (1, 2, 4, 8 and 12 h) with 1 μM SK-3-91, or 1 μM of DB0646 relative to DMSO control. Data are from n=1 biologically independent treatment samples. FIG. 11F is a plot showing correlation of kinase degradability score and reported protein half-life in listed cell types.

FIG. 12A-FIG. 12C are a series of graphs and immunoblots showing comparative analysis of how recruitment of CRBN or VHL impact the kinases degraded. FIG. 12A is a series of graphs of intracellular ligase engagement assay for indicated compounds. BRD4^{BD2}-GFP reporter cells were treated with increasing concentration of lenalidomide or indicated compound for 5 h in the presence of dBET6 1010 (CRBN) or AT1 (VHL). Relative abundance of BRD4^{BD2}-GFP was measured by FACS. Data are represented as means±s.d. of n=3 biologically independent replicates. FIG. 12B is an image of the chemical structures of RSS0628 and RSS0680. FIG. 12C is an image of an immunoblot analysis of MOLT-4 cells treated with RSS0628 or RSS0680 at the indicated dose for 4 h. Data representative of n=2 independent experiments.

FIG. 13A-FIG. 13B are a set of scatterplots showing an assessment of the protein kinases that are degraded through a p97 dependent mechanism. The scatter plots in FIG. 13A-FIG. 13B depict the fold change in relative abundance following a 5-h treatment of MOLT-4 cells with 1 μM of the indicated compounds with (blue) and without (orange) co-treatment with 5 μM of CB-5083, a p97 inhibitor.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “binding” as it relates to interaction between the targeting ligand (moiety of the bifunctional compounds that bind targeted protein/s) and the targeted proteins, which in this invention include AAK1. ABL1, ABL2, AKT2, AKT3, AURK4. AURKA, AURKB, BCKDK, BLK, BMP2K, BMPR1A, BUB1, BUB1B, CAMKK1, CDC7, CDK1, CDK10, CDK11A, CDK11B, CDK12, CDK13, CDK14, CDK16, CDK17, CDK18, CDK2, CDK3, CDK4, CDK5, CDK6, CDK7, CDK9, CHEK1, CIT, CLK1, COQ8A, COQ8B, CSK, CSNK1A1, CSNK1D, CSNK1E, DAPK1, DDR2, EIF2AK2, EIF2AK4, EPHA1, EPHA2, EPHA3, EPHB2, EPHB3, EPHB4, EPHB6, ERN1, FER, FGFR1, FGR2, FYN, GAK, GSK3A, GSK3B, HIPK1, IRAK1, IRAK4, ITK, LATS1, LCK, LIMK1, LIMK2, LRRK2, LYN, MAP2K5, MAP3K1, MAP3K11, MAP3K12, MAP3K20, MAP3K21, MAP3K7, MAP4K1, MAP4K2, MAP4K3, MAP4K5, MAPK11, MAPK12, MAPK14, MAPK6, MAPK7, MAPK8, MAPK9, MAPKAPK2, MAPKAPK3, MAPKAPK5, MARK2, MARK3, MARK4, MAST3, MELK, MINK1, MKNK2, NEK2, NEK9, NLK, NUAK1, PAK4, PDIK1L, PDK1, PDK2, PDK3, PIK3CG, PIM2, PKMYT1, PKN3, PLK1, PLK4, PRAG1, PRKAA1, PRKAA2, PTK2, PTK2B. PTK6, RIOK2, RIPK1, RIPK2. RPS6KAL. RPS6KA3, RPS6KA4, RPS6KA6, RPS6KB1, RPS6KC1, SBK1, SGK3, SIK2, SIK3, SIKA2, SNRK, SRC, STK10, STK17A, STK17B, STK32C, STK33, STK35, STK38, STK4, STK40, TAOK2, TAOK3, TEC, TESK2, TGFBR1, TNK1, TNK2, TRIB3, TRPM7, TTK, TYK2, UHMK1, ULK1, ULK3, WEE1, and YES1, typically refers to an inter-molecular interaction that is preferential (also referred to herein as “selective”) in that binding of the targeting ligand with other proteins present in the cell, including other isoforms, is substantially less and in some cases may be functionally insignificant, at least from the standpoint of degradation.

The term “binding” as it relates to interaction between the degron (moiety of the bifunctional compounds that binds an E3 ubiquitin ligase) the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, bifunctional compounds (also referred to herein as degraders) for targeted kinase degradation are represented by any of the following structures:

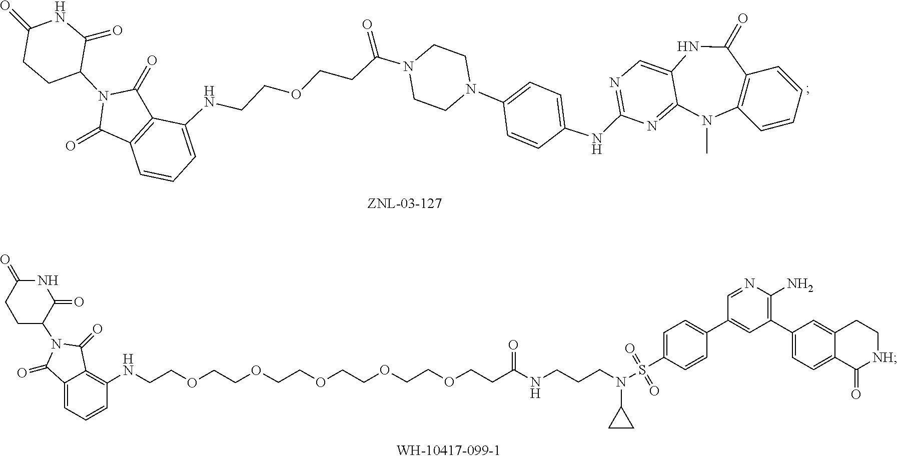 [SMILES: CN1c2ccccc2C(=O)Nc2cnc(Nc3ccc(N4CCN(C(=O)CCOCCNc5cccc6c5C(=O)N(C5CCC(=O)NC5=O)C6=O)CC4)cc3)nc21 ; Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2)cc1-c1ccc2c(c1)CCNC2=O]

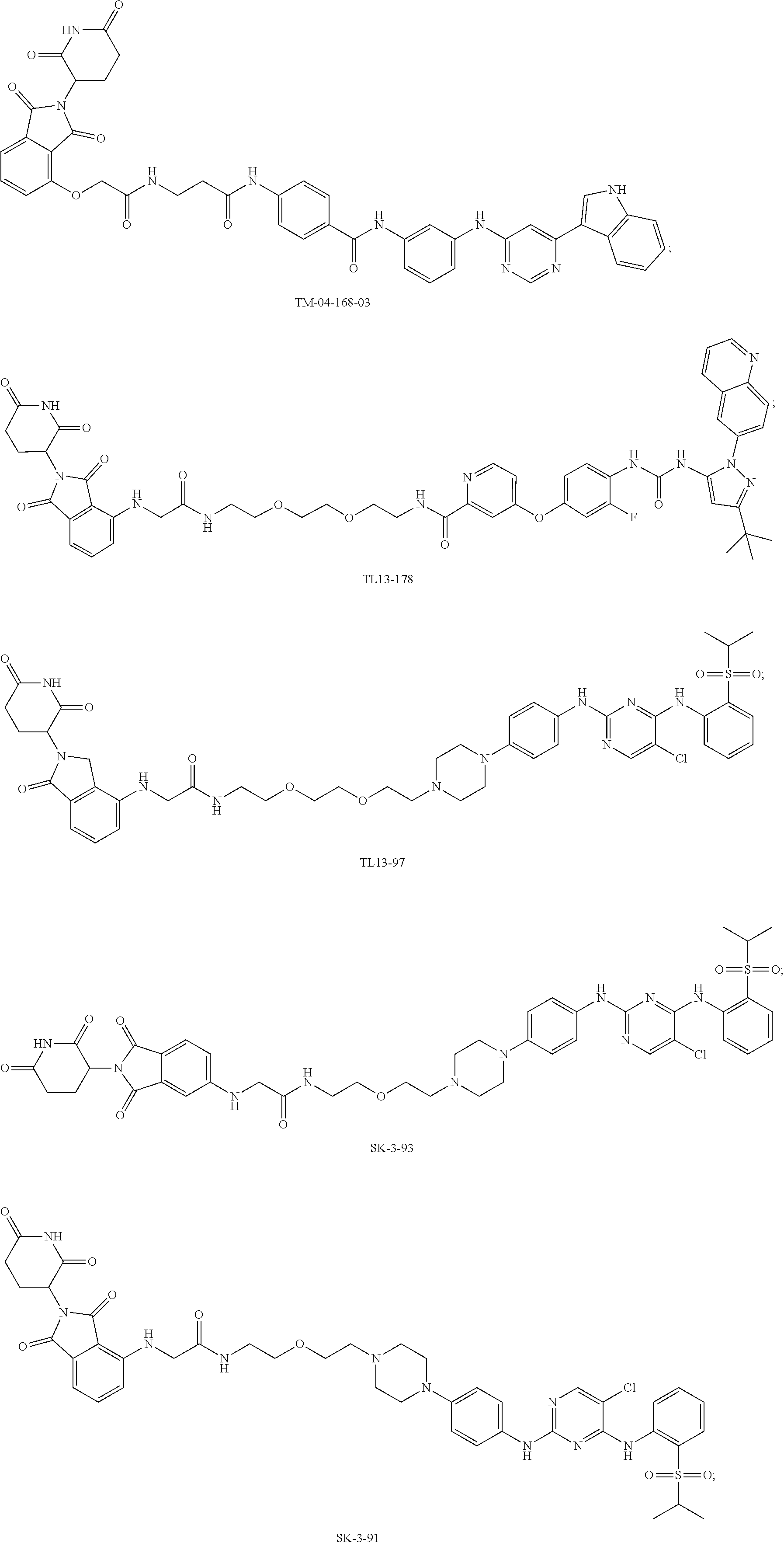

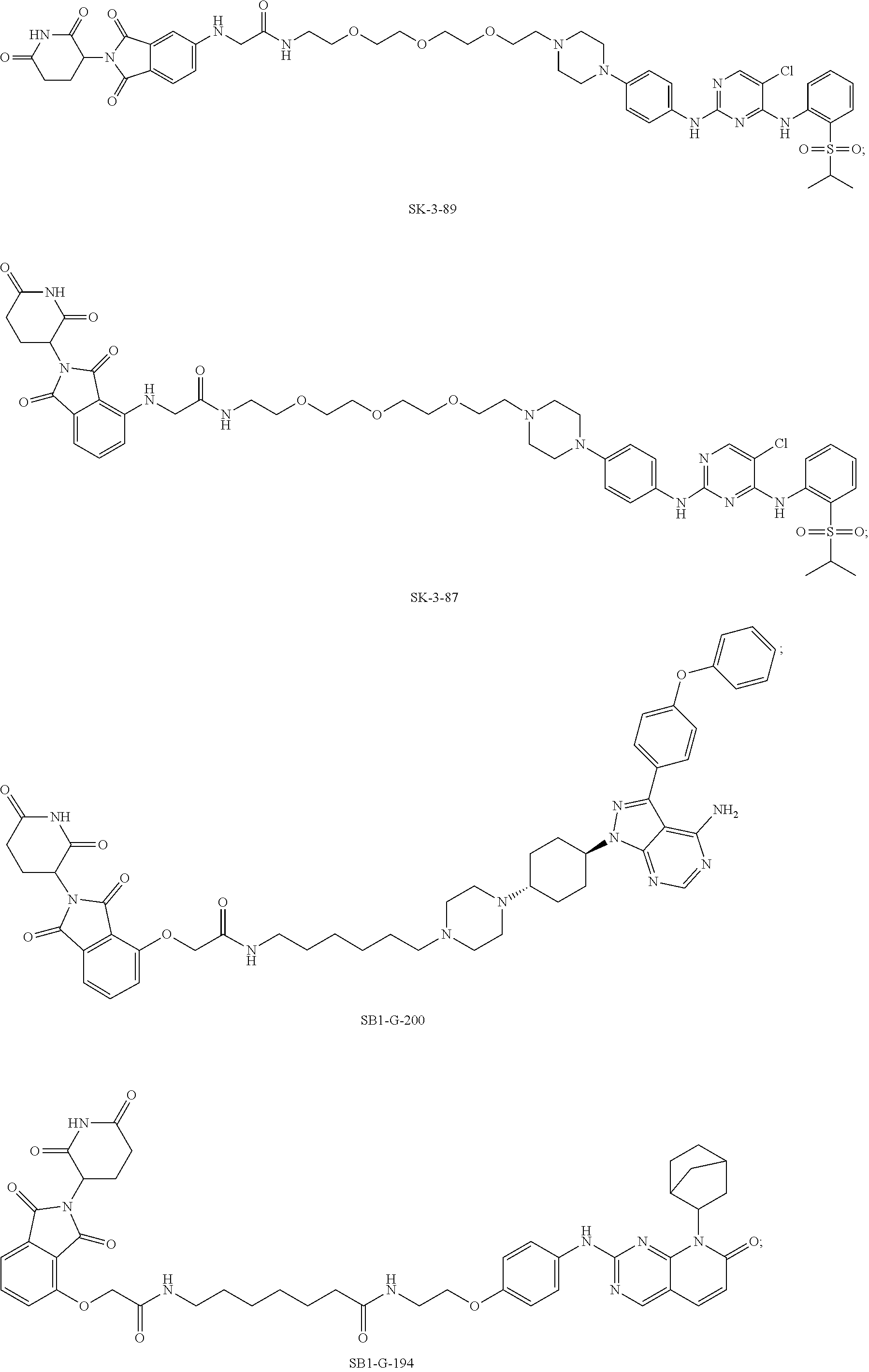 [SMILES: CC(C)S(=O)(=O)c1ccccc1Nc1nc(Nc2ccc(N3CCN(CCOCCOCCOCCNC(=O)CNc4ccc5c(c4)C(=O)N(C4CCC(=O)NC4=O)C5=O)CC3)cc2)ncc1Cl ; CC(C)S(=O)(=O)c1ccccc1Nc1nc(Nc2ccc(N3CCN(CCOCCOCCOCCNC(=O)CNc4cccc5c4C(=O)N(C4CCC(=O)NC4=O)C5=O)CC3)cc2)ncc1Cl ; Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1 ; O=C(CCCCCCNC(=O)COc1cccc2c1C(=O)N(C1CCC(=O)NC1=O)C2=O)NCCOc1ccc(Nc2ncc3ccc(=O)n(C4CC5CCC4C5)c3n2)cc1]

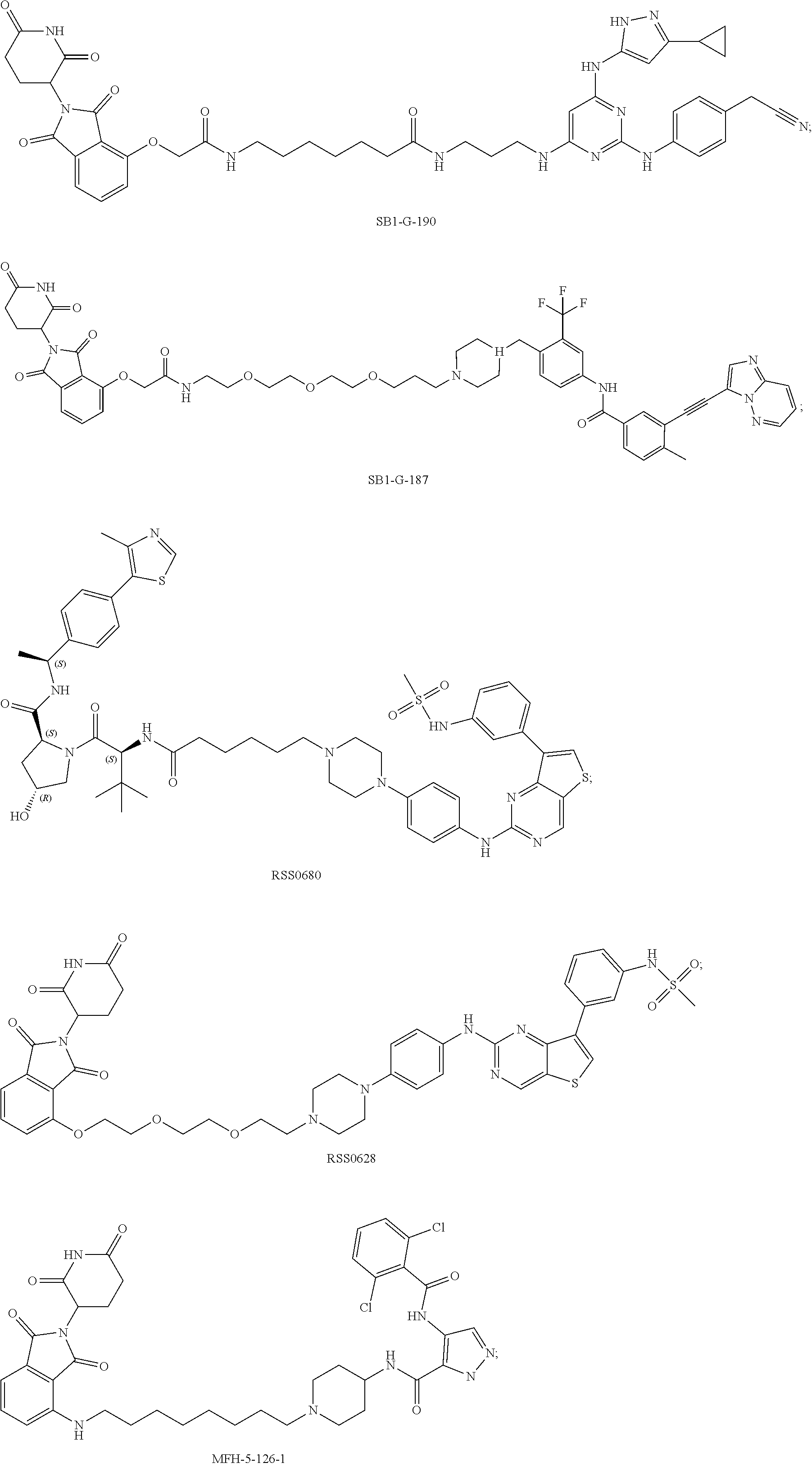

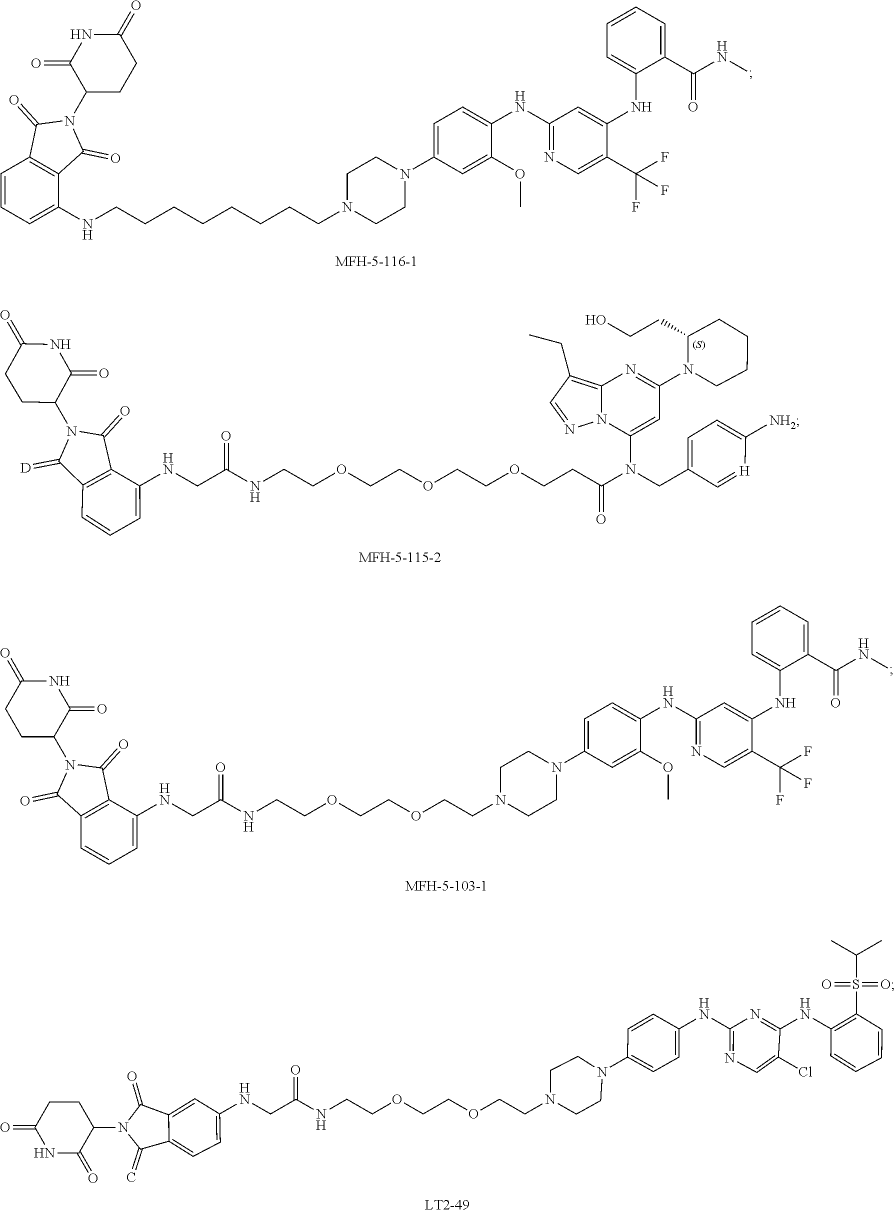

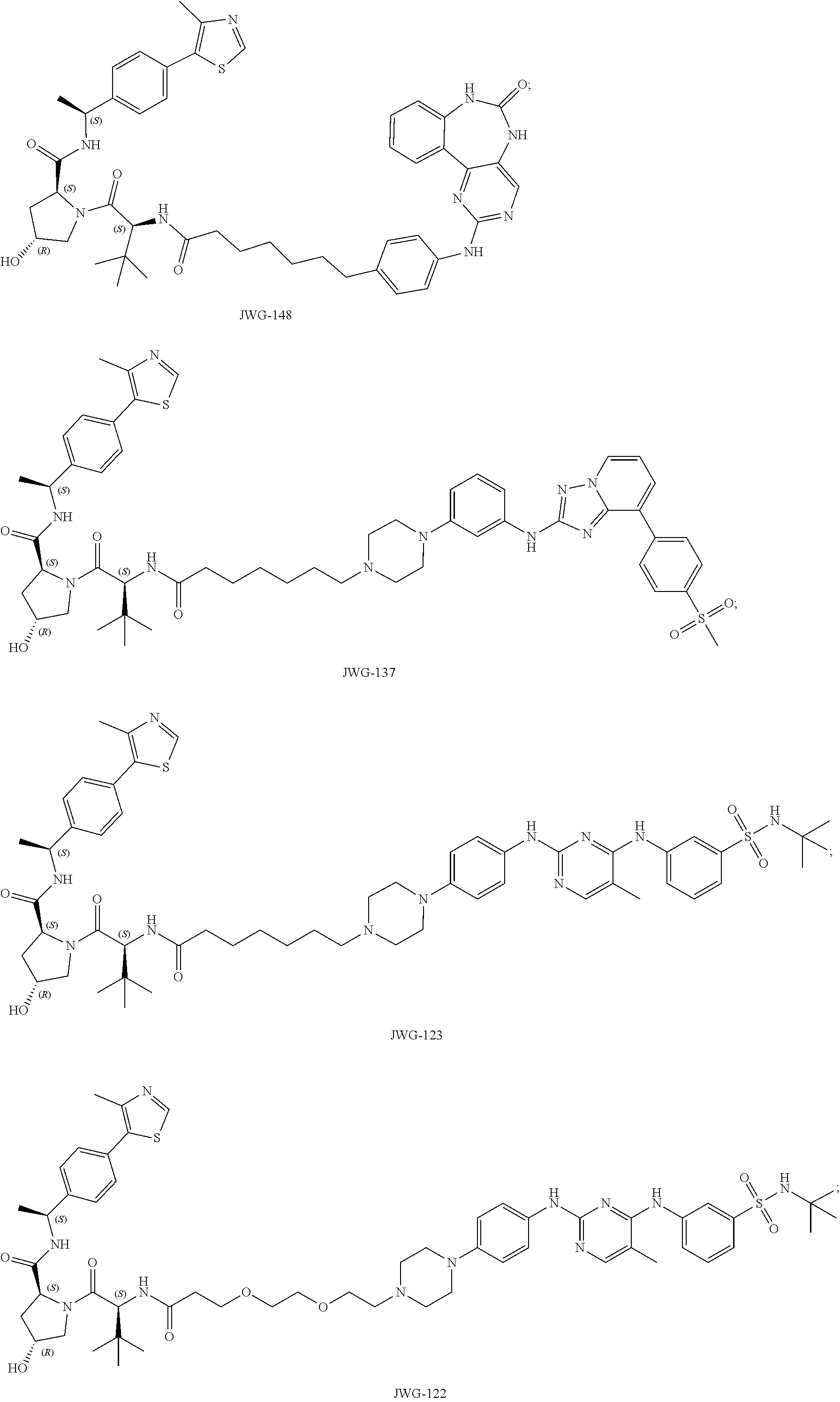

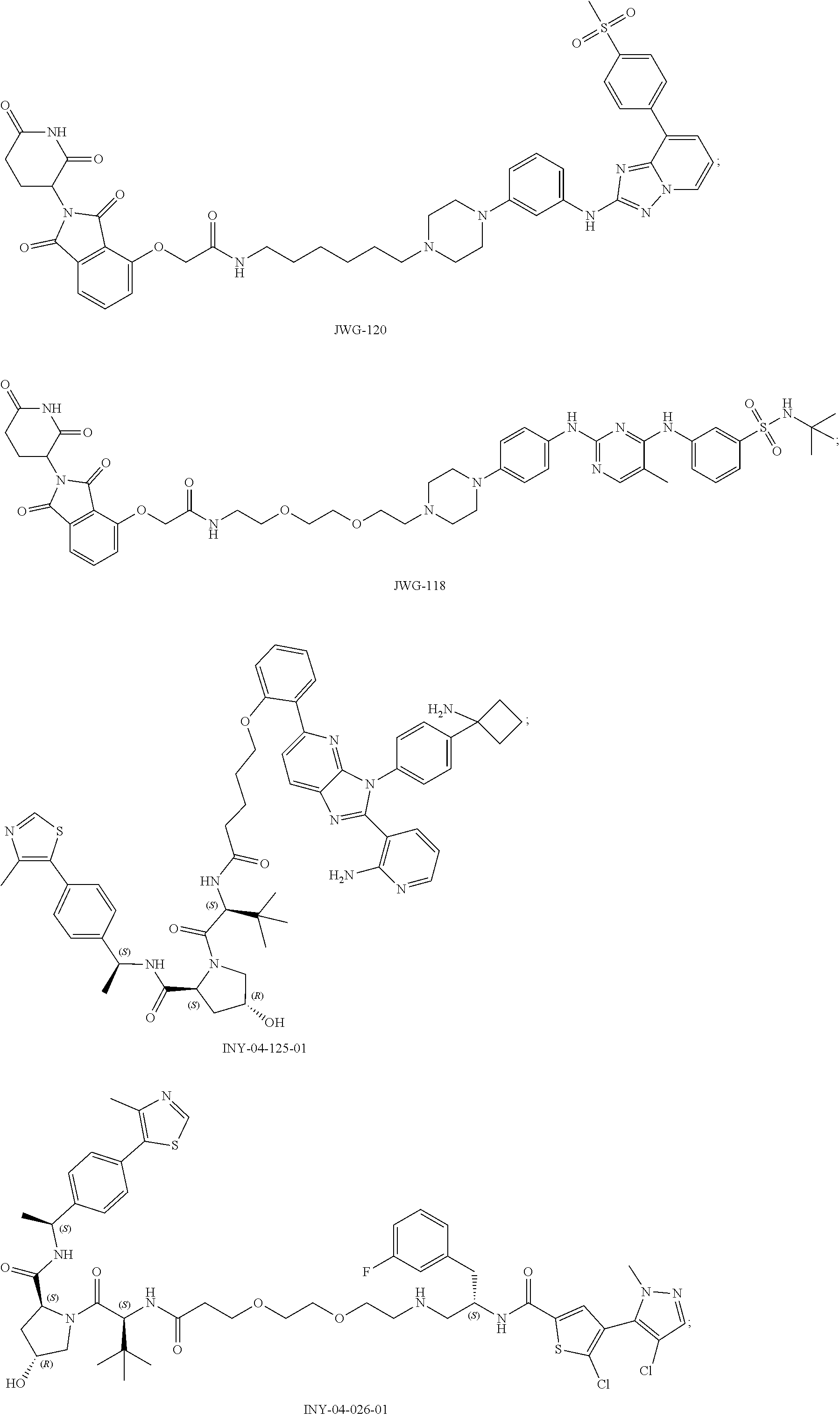

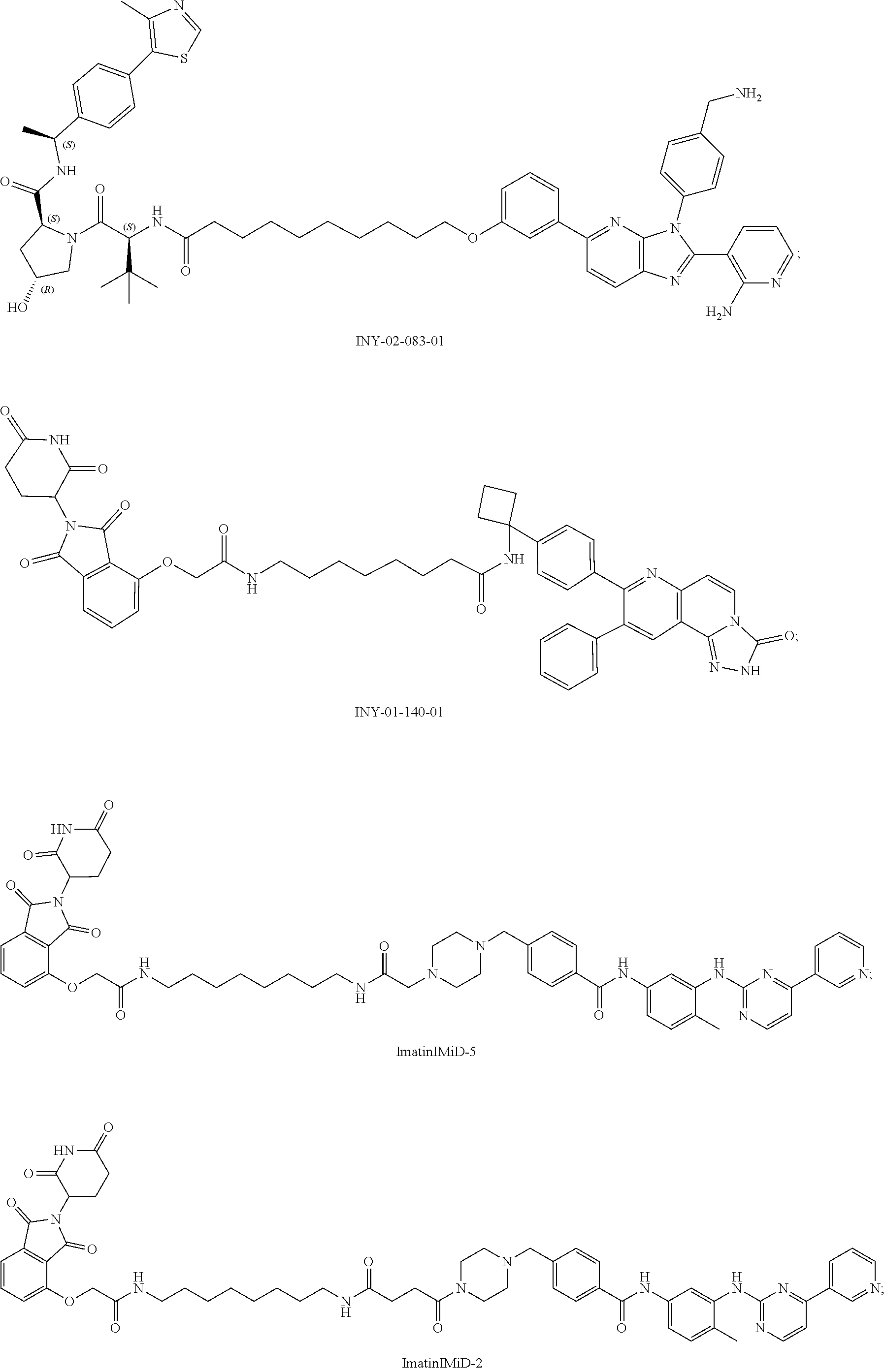

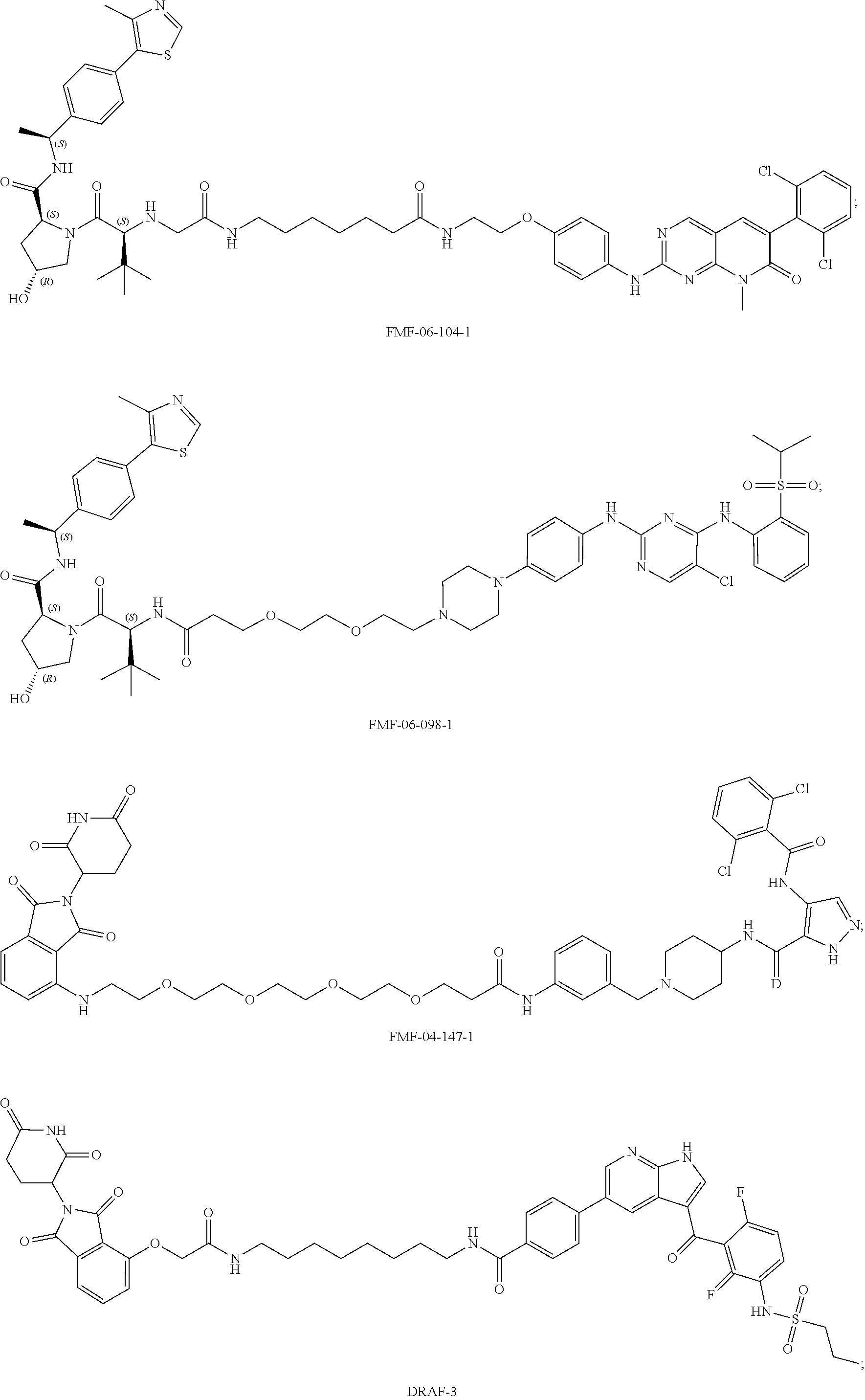

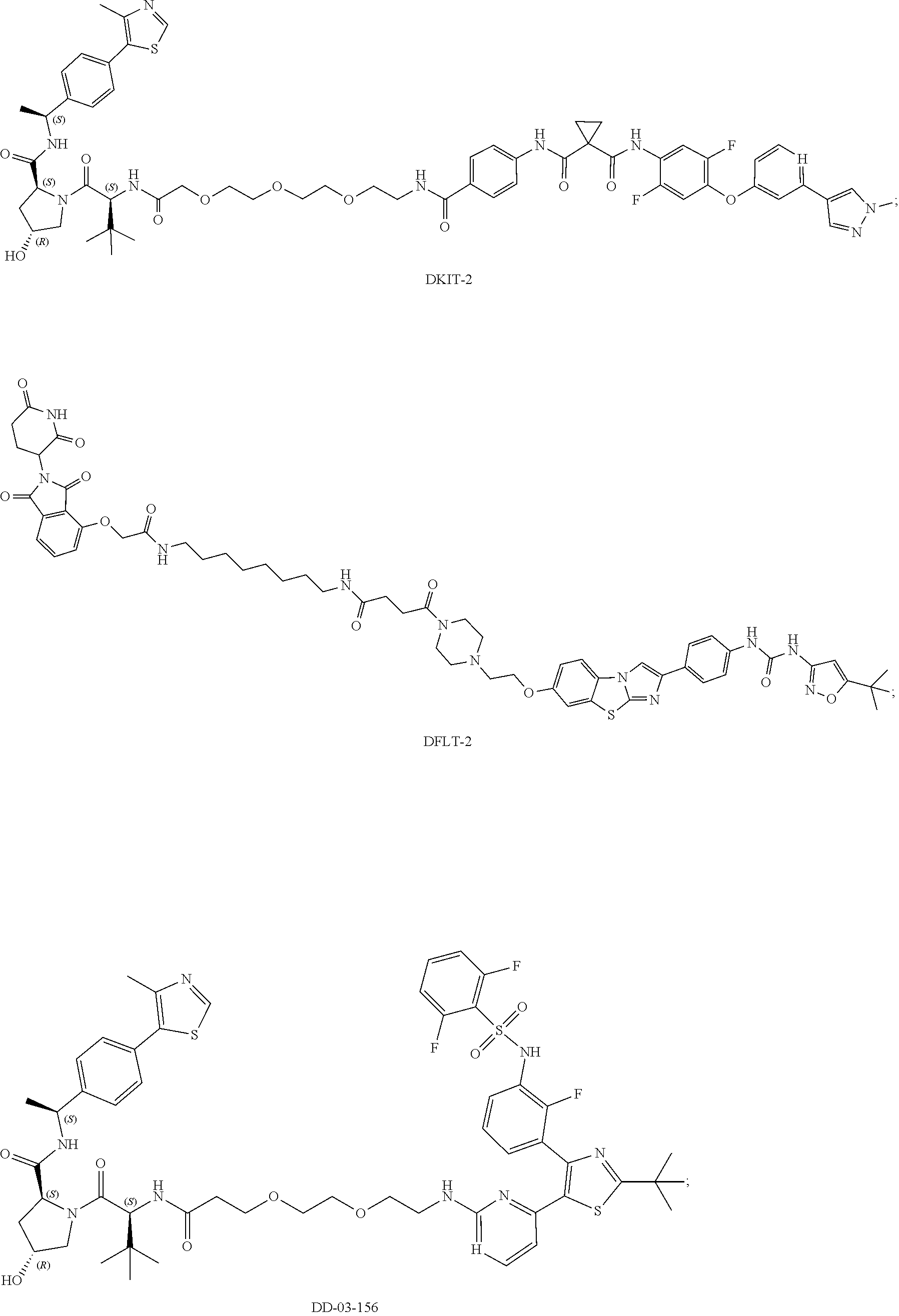

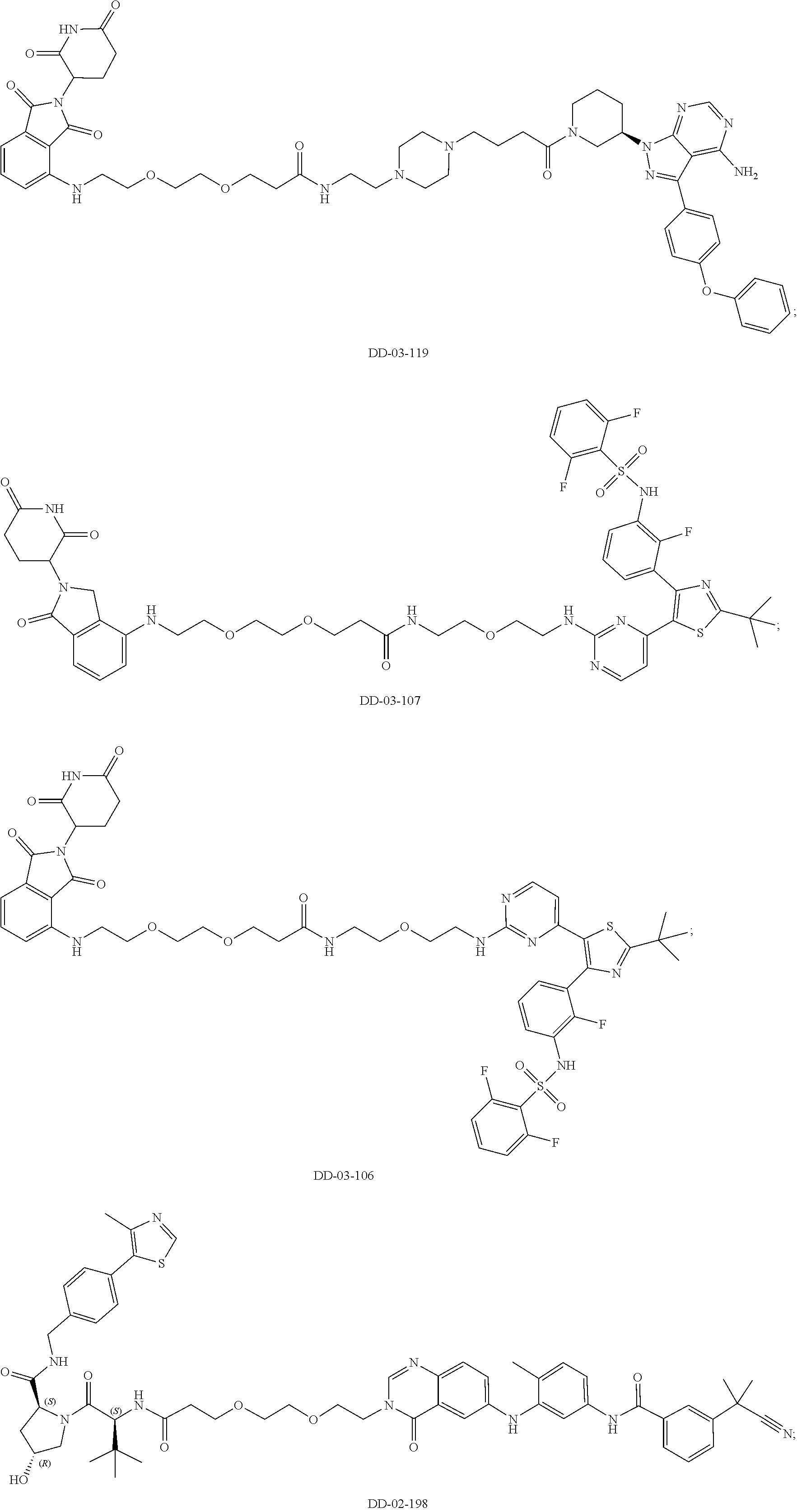

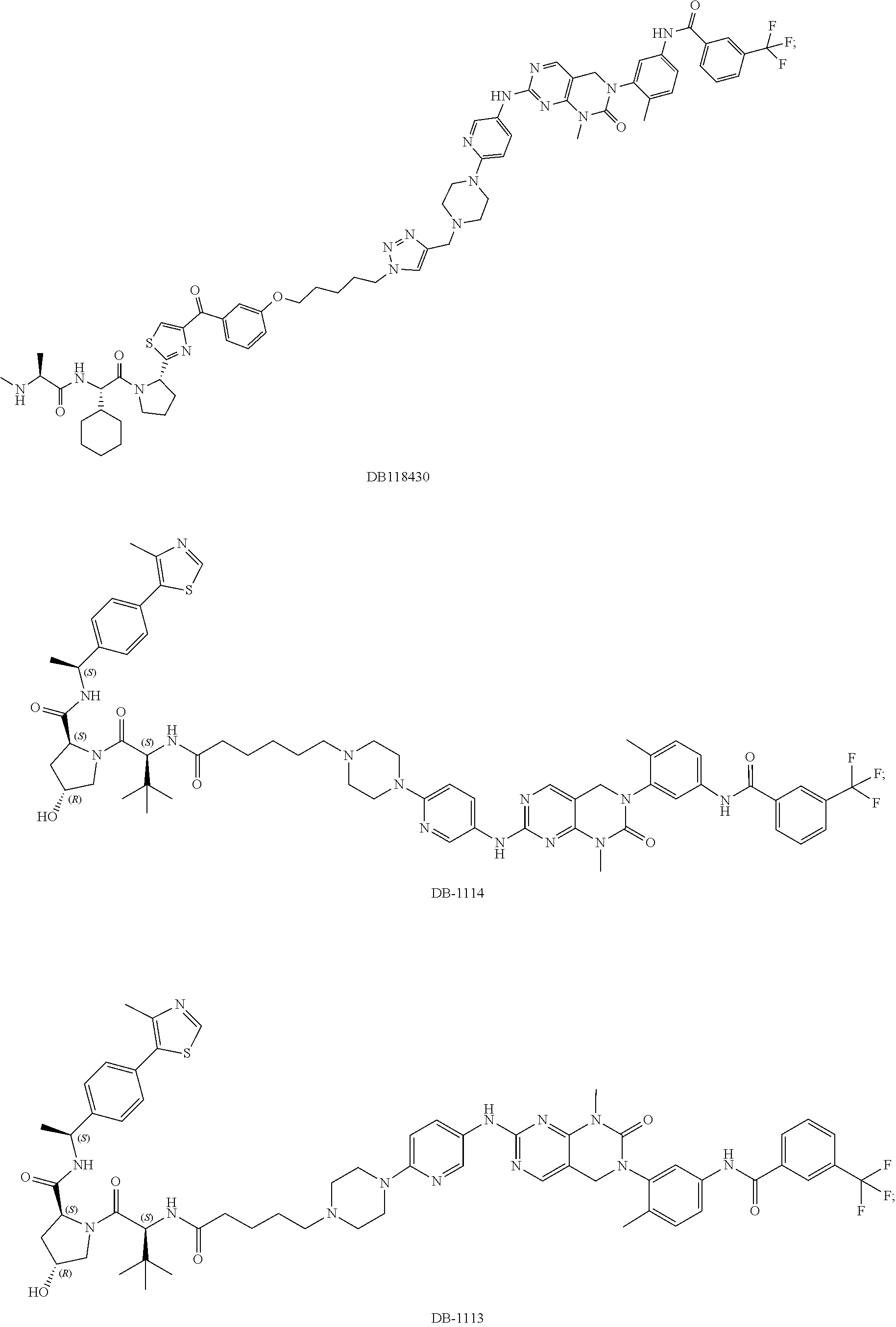

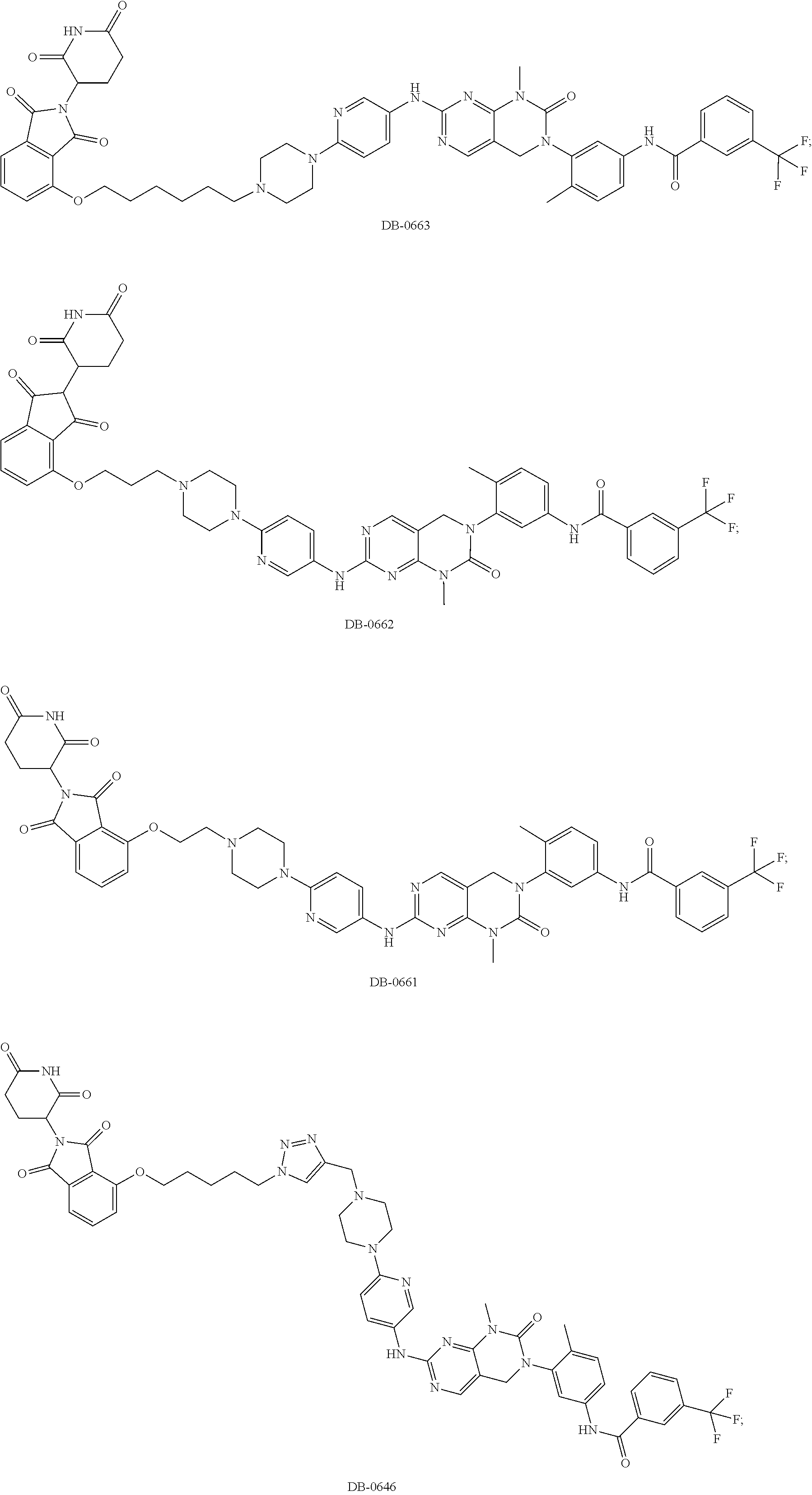 [SMILES: Cc1ccc(NC(=O)c2cccc(C(F)(F)F)c2)cc1N1Cc2cnc(Nc3ccc(N4CCN(CCCCCCOc5cccc6c5C(=O)N(C5CCC(=O)NC5=O)C6=O)CC4)nc3)nc2N(C)C1=O ; Cc1ccc(NC(=O)c2cccc(C(F)(F)F)c2)cc1N1Cc2cnc(Nc3ccc(N4CCN(CCCOc5cccc6c5C(=O)C(C5CCC(=O)NC5=O)C6=O)CC4)nc3)nc2N(C)C1=O ; Cc1ccc(NC(=O)c2cccc(C(F)(F)F)c2)cc1N1Cc2cnc(Nc3ccc(N4CCN(CCOc5cccc6c5C(=O)N(C5CCC(=O)NC5=O)C6=O)CC4)nc3)nc2N(C)C1=O ; Cc1ccc(NC(=O)c2cccc(C(F)(F)F)c2)cc1N1Cc2cnc(Nc3ccc(N4CCN(Cc5cn(CCCCCOc6cccc7c6C(=O)N(C6CCC(=O)NC6=O)C7=O)nn5)CC4)nc3)nc2N(C)C1=O]

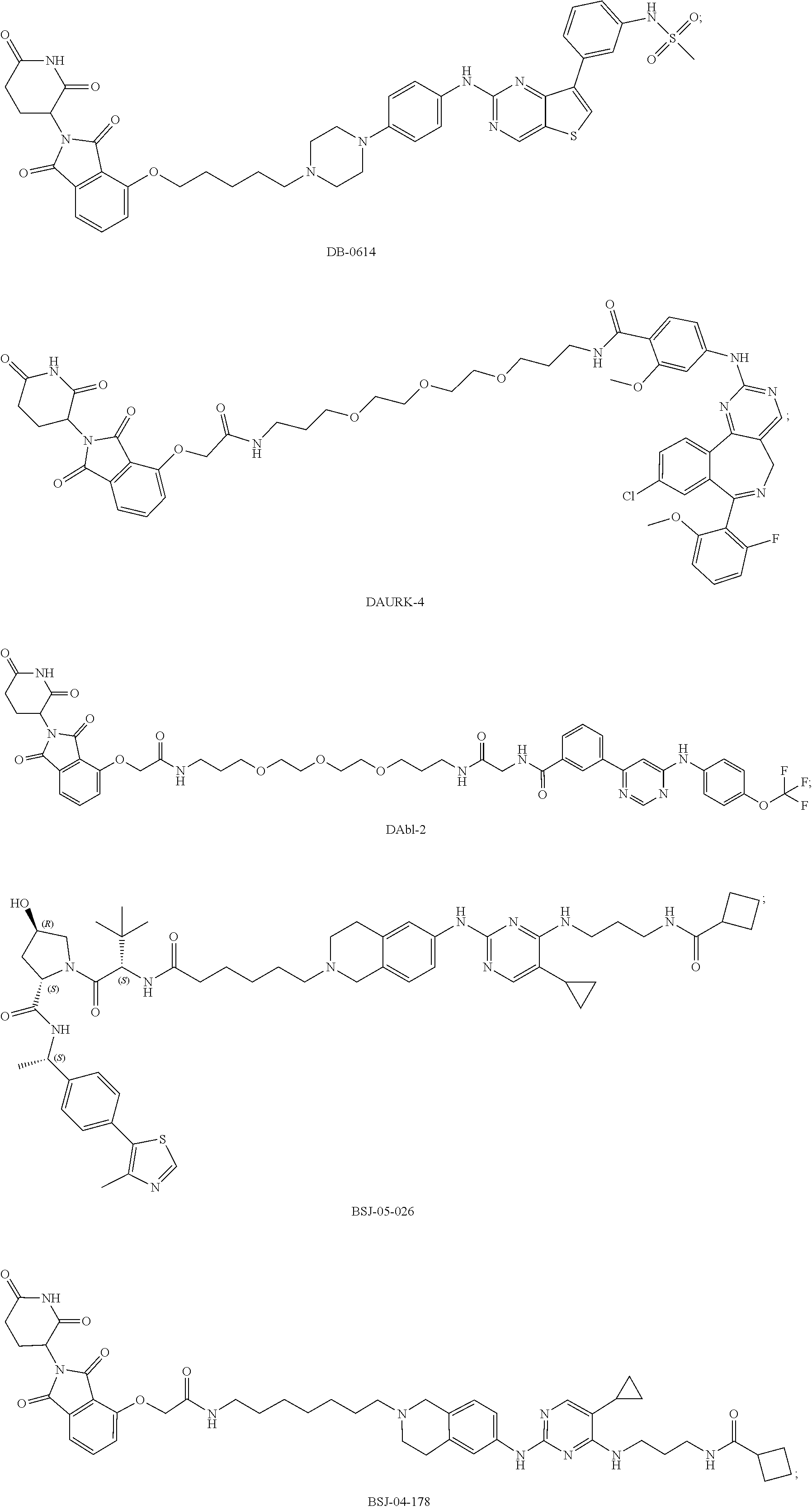

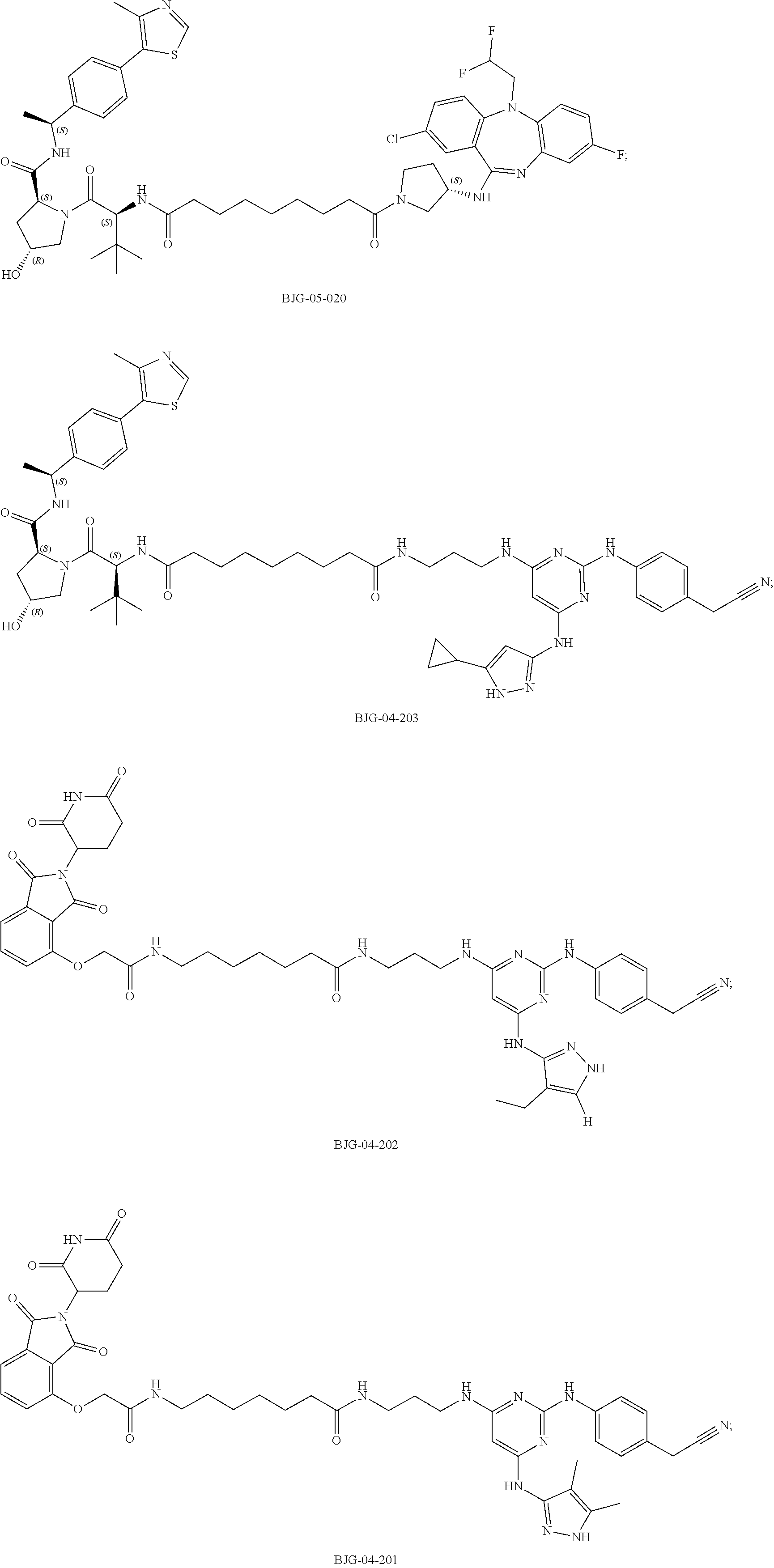

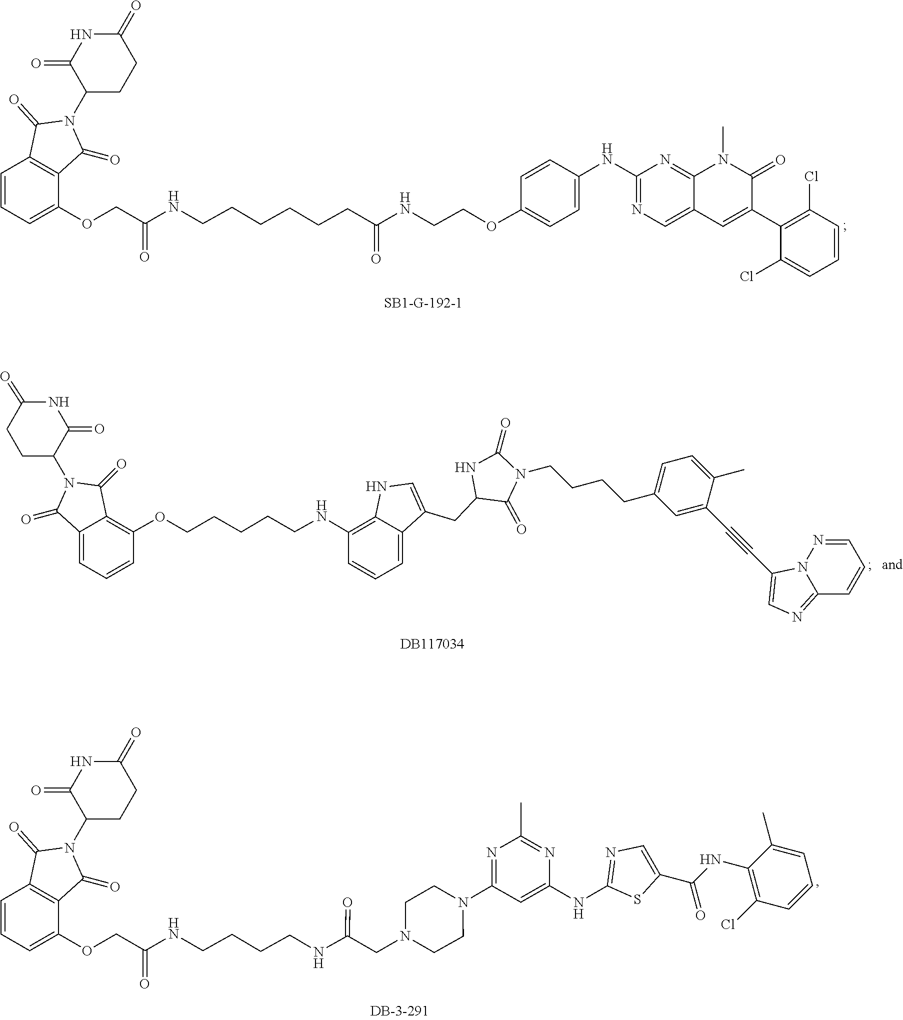 [SMILES: Cn1c(=O)c(-c2c(Cl)cccc2Cl)cc2cnc(Nc3ccc(OCCNC(=O)CCCCCCNC(=O)COc4cccc5c4C(=O)N(C4CCC(=O)NC4=O)C5=O)cc3)nc21 ; Cc1ccc(CCCCN2C(=O)NC(Cc3c[nH]c4c(NCCCCCOc5cccc6c5C(=O)N(C5CCC(=O)NC5=O)C6=O)cccc34)C2=O)cc1C#Cc1cnc2cccnn12 ; Cc1nc(Nc2ncc(C(=O)Nc3c(C)cccc3Cl)s2)cc(N2CCN(CC(=O)NCCCCNC(=O)COc3cccc4c3C(=O)N(C3CCC(=O)NC3=O)C4=O)CC2)n1]

and pharmaceutically acceptable salts or stereoisomers thereof.

In some embodiments, the bifunctional compound degrades BLK, LIMK1, LIMK2, STK17A, and TNK2, and is represented by structure:

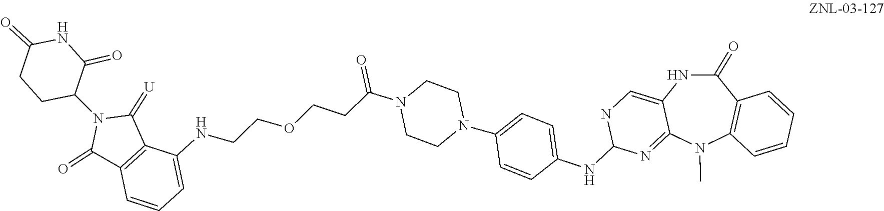

or a pharmaceutically acceptable salt or stereoisomer thereof.

In some embodiments, the bifunctional compound degrades CDK14, CSNK1A1, CSNK1D, CSNK1E, GSK3A, GSK3B, LIMK2, MAP3K1, MINK1, NUAK1, PAK4, PIM2, STK10, STK17B, STK35, and STK4, and is represented by structure:

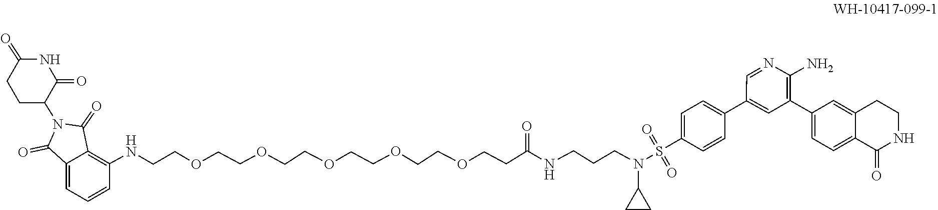 [SMILES: Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2)cc1-c1ccc2c(c1)CCNC2=O]

or a pharmaceutically acceptable salt or stereoisomer thereof.

In some embodiments, the bifunctional compound degrades CDK4, LIMK1, MAP3K20, MAPK14, and MAST3, and is represented by structure:

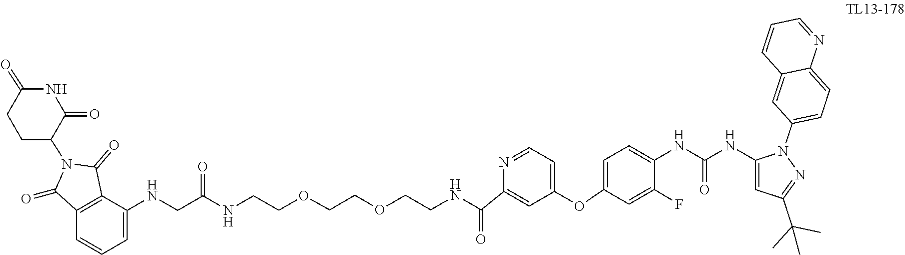 [SMILES: CC(C)(C)c1cc(NC(=O)Nc2ccc(Oc3ccnc(C(=O)NCCOCCOCCNC(=O)CNc4cccc5c4C(=O)N(C4CCC(=O)NC4=O)C5=O)c3)cc2F)n(-c2ccc3ncccc3c2)n1]

or a pharmaceutically acceptable salt or stereoisomer thereof.

In some embodiments, the bifunctional compound degrades AAK1, ABL2, AURKA, AURKB, BLK, BUB1B, CDK13, CDK17, CDK2, CDK4, CDK5. CDK6. CDK7. CDK9. FER, ITK, LCK, LIMK1, LIMK2, MAP3K11, MARK4, PLK4, PRKAA1, RPS6KA1, SRC. STK10, STK38, TEC, TNK2, ULK1, ULK3, and WEE1, and is represented by structure:

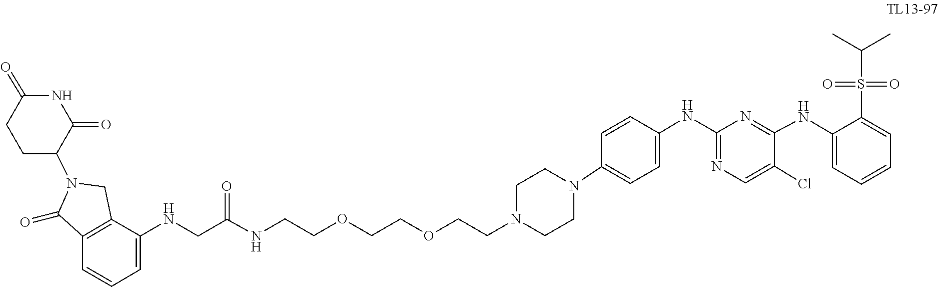 [SMILES: CC(C)S(=O)(=O)c1ccccc1Nc1nc(Nc2ccc(N3CCN(CCOCCOCCNC(=O)CNc4cccc5c4CN(C4CCC(=O)NC4=O)C5=O)CC3)cc2)ncc1Cl]

or a pharmaceutically acceptable salt or stereoisomer thereof.

In some embodiments, the bifunctional compound degrades AAK1, ABL2, AURKA, BLK, BMP2K, CDK12, CDK13, CDK17, CDK2, CDK4, CDK5, CDK6, CDK7, CDK9, FER, GAK, ITK. LCK, LIMK2. PRKAA1, PTK2B, RPS6KA1, SRC, and WEE1, and is represented by structure:

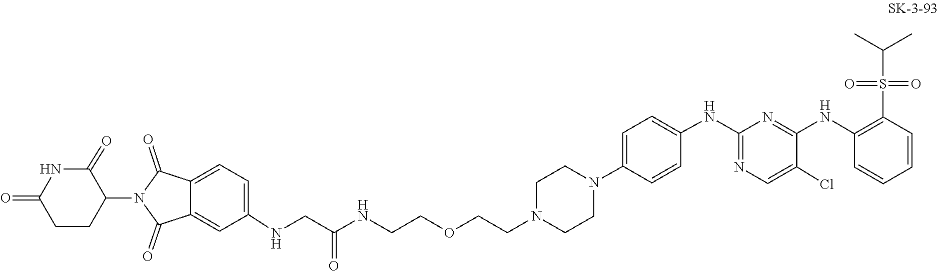 [SMILES: CC(C)S(=O)(=O)c1ccccc1Nc1nc(Nc2ccc(N3CCN(CCOCCNC(=O)CNc4ccc5c(c4)C(=O)N(C4CCC(=O)NC4=O)C5=O)CC3)cc2)ncc1Cl]

or a pharmaceutically acceptable salt or stereoisomer thereof.

In some embodiments, the bifunctional compound degrades AAK1, ABL1, ABL2, AKT2, AKT3, AURKA, AURKB, BCKDK, BLK, BMP2K, BMPR1A, BUB1, BUB1B, CDC7, CDK1O, CDK12, CDK13, CDK14, CDK16, CDK17, CDK18, CDK2, CDK4, CDK5, CDK6, CDK7, CDK9, CO18A, CSK, CSNK1D. EPHB2, EPHB4, FER, FYN, GAK, HIPK1, ITK, LATS1, LCK, LIMK1, LIMK2, LRRK2, MAP3K1, MAP3K11, MAP3K12, MAP3K21, MAP4K1, MAP4K3, MAPK6, MAPK7, MARK2, MARK4, MAST3, MKNK2, NEK2, PDK3, PLK1, PLK4, PRAG1, PRKAA1, PRKAA2, PTK2, PTK2B, PTK6, RIOK2, RPS6KA1, RPS6KA6, RPS6KB1, RPS6KC1, SBK1, SIK2, SRC. STK17A, STK17B, STK32C, STK33, STK40, TEC, TGFBR1, TNK1, TNK2, TRIB3, TRPM7, TTK, UHMK1, ULK1, ULK3, WEE1, and YES1, and is represented by structure:

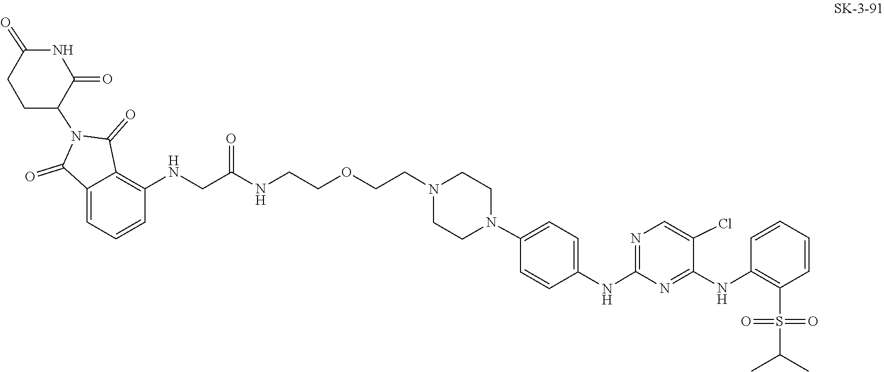 [SMILES: CC(C)S(=O)(=O)c1ccccc1Nc1nc(Nc2ccc(N3CCN(CCOCCNC(=O)CNc4cccc5c4C(=O)N(C4CCC(=O)NC4=O)C5=O)CC3)cc2)ncc1Cl]

or a pharmaceutically acceptable salt or stereoisomer thereof.

In some embodiments, the bifunctional compound degrades AAK1, AURKA, BLK, CDK12, CDK17, CDK2, CDK4, CDK5, CDK6, CDK7, CDK9, FER, ITK, LCK, LIMK2, PTK2B, STK10, and WEE1, and is represented by structure:

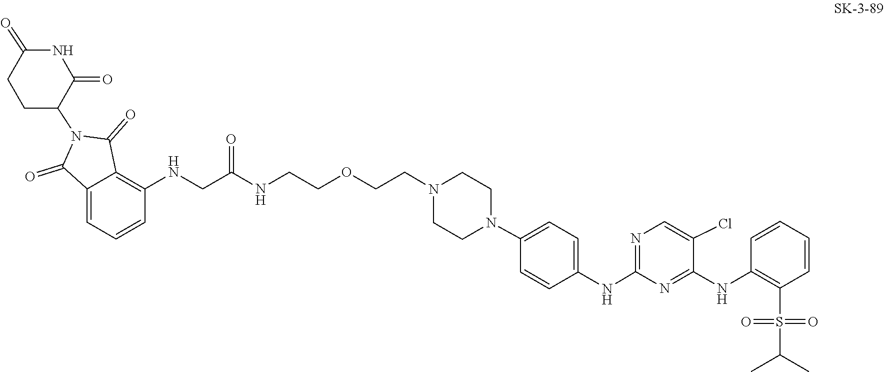 [SMILES: CC(C)S(=O)(=O)c1ccccc1Nc1nc(Nc2ccc(N3CCN(CCOCCNC(=O)CNc4cccc5c4C(=O)N(C4CCC(=O)NC4=O)C5=O)CC3)cc2)ncc1Cl]

or a pharmaceutically acceptable salt or stereoisomer thereof.

In some embodiments, the bifunctional compound degrades AAK1, AURKA, BLK, CDK12, CDK17, CDK2, CDK5, CDK7, CDK9, FER, ITK, LCK, LIMK2, PTK2B, and WEE1, and is represented by structure:

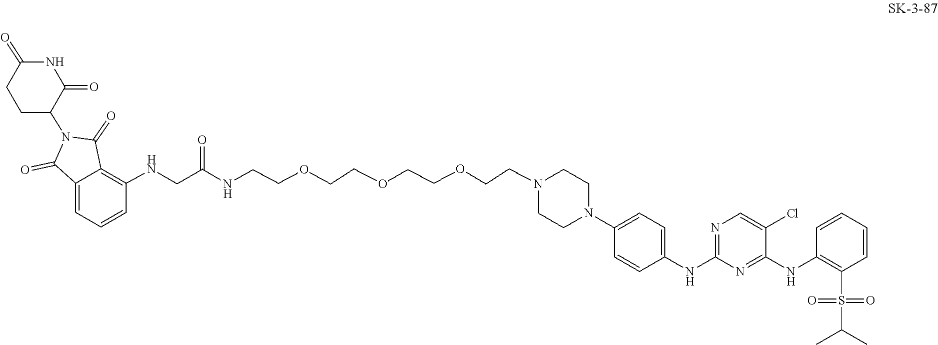 [SMILES: CC(C)S(=O)(=O)c1ccccc1Nc1nc(Nc2ccc(N3CCN(CCOCCOCCOCCNC(=O)CNc4cccc5c4C(=O)N(C4CCC(=O)NC4=O)C5=O)CC3)cc2)ncc1Cl]

or a pharmaceutically acceptable salt or stereoisomer thereof.

In some embodiments, the bifunctional compound degrades ABL1, ABL2, BLK, CSNK1E. CSK, FYN, LATS1, LCK, LIMK1, MAP2K5, and SRC, and is represented by structure:

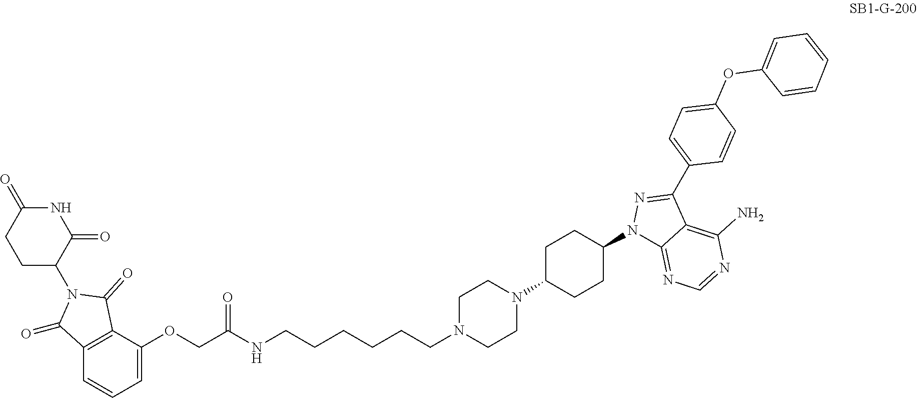 [SMILES: Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1]

or a pharmaceutically acceptable salt or stereoisomer thereof.

In some embodiments, the bifunctional compound degrades AAK1, AURKA, AURKB, CDK6, CDK9, FGR2, STK17A, and TTK, and is represented by structure:

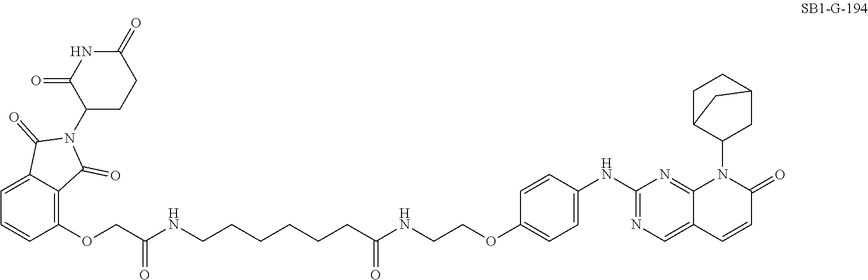 [SMILES: O=C(CCCCCCNC(=O)COc1cccc2c1C(=O)N(C1CCC(=O)NC1=O)C2=O)NCCOc1ccc(Nc2ncc3ccc(=O)n(C4CC5CCC4C5)c3n2)cc1]

or a pharmaceutically acceptable salt or stereoisomer thereof.

In some embodiments, the bifunctional compound degrades AAK1, AURKA, BMP2K, GSK3A, and GSK3B, and is represented by structure:

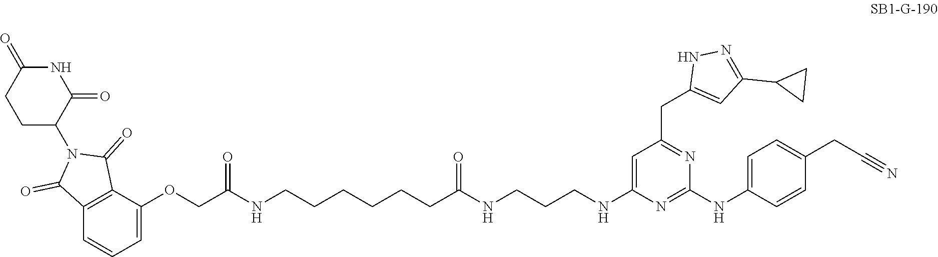 [SMILES: N#CCc1ccc(Nc2nc(Cc3cc(C4CC4)n[nH]3)cc(NCCCNC(=O)CCCCCCNC(=O)COc3cccc4c3C(=O)N(C3CCC(=O)NC3=O)C4=O)n2)cc1]

or a pharmaceutically acceptable salt or stereoisomer thereof.

In some embodiments, the bifunctional compound degrades ABL1, ABL2, BLK, CDK14, CDK17, CDK5. CDK6, COQ8A, EPHA1, EPHA2, FER, FYN, GAK, IRAK1, LCK, LYN, MAP3K1, MAP3K20, MAP3K7, MAP4K2, MAP4K5, MAPK14, PDK1, PDK2, PDK3, RIPK1, RIPK2, SRC, STK10, TAOK3, and YES1, and is represented by structure:

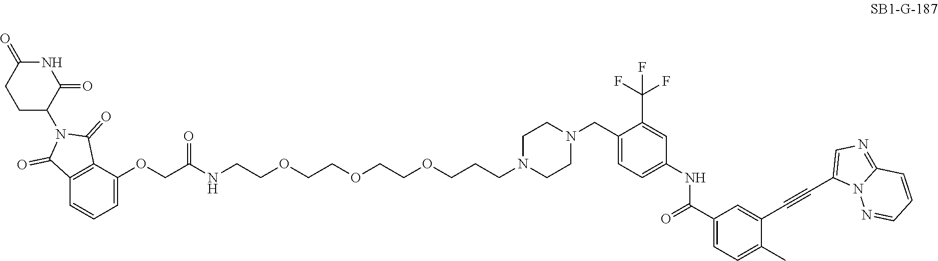 [SMILES: Cc1ccc(C(=O)Nc2ccc(CN3CCN(CCCOCCOCCOCCNC(=O)COc4cccc5c4C(=O)N(C4CCC(=O)NC4=O)C5=O)CC3)c(C(F)(F)F)c2)cc1C#Cc1cnc2cccnn12]

or a pharmaceutically acceptable salt or stereoisomer thereof.

In some embodiments, the bifunctional compound degrades AAK1, CDK1, CDK16, CDK2, CDK4, CDK6, EIF2AK4, GAK. LATS1, LIMK2, MAPK6, MAPKAPK5, MARK2, MARK4, MKNK2, NEK9, RPS6KB1, SIK2, SNRK, STK17A, STK17B, STK35, and WEE1, and is represented by structure:

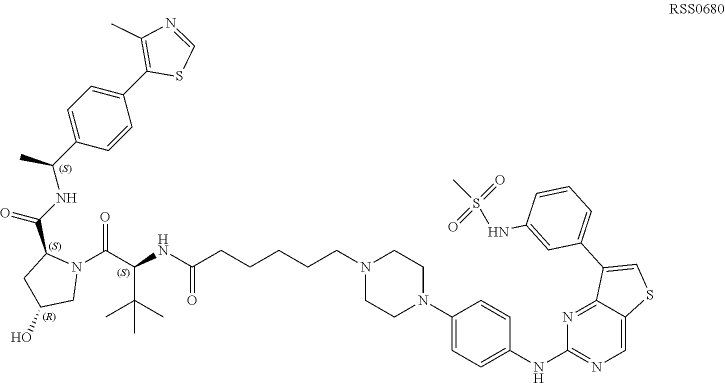

or a pharmaceutically acceptable salt or stereoisomer thereof.

In some embodiments, the bifunctional compound degrades AAK1. AURKA, CAMKK1, CDK4, CDK6, LIMK2, NEK9, PTK2B, STK17A, STK17B, ULK1, ULK3, and WEE1, and is represented by structure:

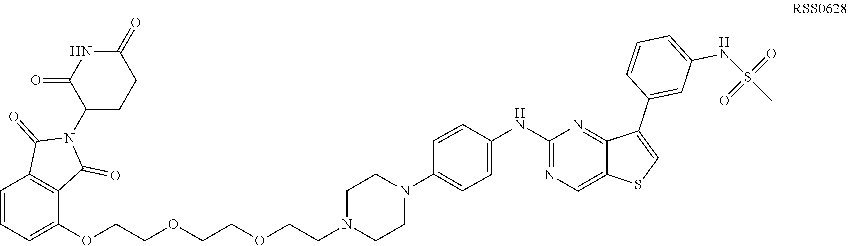 [SMILES: CS(=O)(=O)Nc1cccc(-c2csc3cnc(Nc4ccc(N5CCN(CCOCCOCCOc6cccc7c6C(=O)N(C6CCC(=O)NC6=O)C7=O)CC5)cc4)nc23)c1]

or a pharmaceutically acceptable salt or stereoisomer thereof.

In some embodiments, the bifunctional compound degrades AURKA, BUB1, BUB1B, CDK13, CDK14, CDK17, CDK4, CDK9, CHEK1, CLK1, CSNK1A1, CSNK1D, DAPK1, ERN1, GSK3A, GSK3B, MAP3K1, NUAK1, PIK3CG, PIM2, PLK1, RIOK2, STK17A, STK7B, TTK, UHMK1, and WEE1, and is represented by structure:

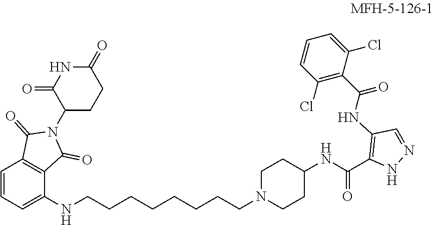 [SMILES: O=C1CCC(N2C(=O)c3cccc(NCCCCCCCCN4CCC(NC(=O)c5[nH]ncc5NC(=O)c5c(Cl)cccc5Cl)CC4)c3C2=O)C(=O)N1]

or a pharmaceutically acceptable salt or stereoisomer thereof.

In some embodiments, the bifunctional compound degrades AURKA, NUAK1, PTK2B, RPS6KA1, RPS6KA3, STK33, and WEE1, and is represented by structure:

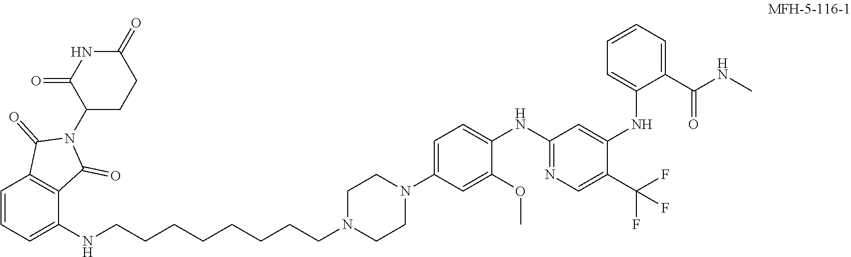 [SMILES: CNC(=O)c1ccccc1Nc1cc(Nc2ccc(N3CCN(CCCCCCCCNc4cccc5c4C(=O)N(C4CCC(=O)NC4=O)C5=O)CC3)cc2OC)ncc1C(F)(F)F]

or a pharmaceutically acceptable salt or stereoisomer thereof.

In some embodiments, the bifunctional compound degrades CDK4. AURK4. WEE1. STK17A, PLK1, BUB1, TTK, UHMK1, MAP3K1, BUB1B, RIOK2, NUAK1, PIM2, and CSNK1A1, and is represented by structure:

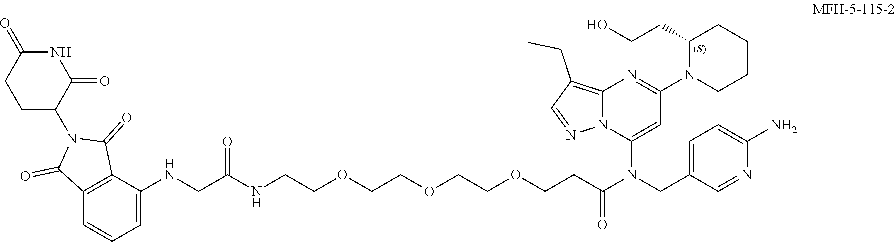

or a pharmaceutically acceptable salt or stereoisomer thereof.

In some embodiments, the bifunctional compound degrades AURKA, CDK1O, CDK7, MAPK7, PTK2B, RPS6KA1, RPS6KA3, STK33, and WEE1, and is represented by structure:

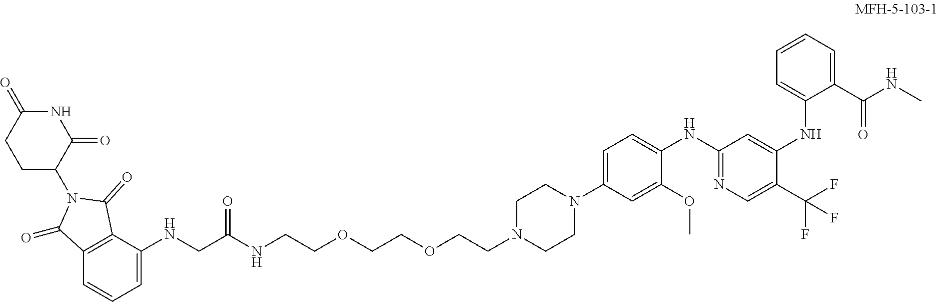 [SMILES: CNC(=O)c1ccccc1Nc1cc(Nc2ccc(N3CCN(CCOCCOCCNC(=O)CNc4cccc5c4C(=O)N(C4CCC(=O)NC4=O)C5=O)CC3)cc2OC)ncc1C(F)(F)F]

or a pharmaceutically acceptable salt or stereoisomer thereof.

In some embodiments, the bifunctional compound degrades CDK4, AURKA, WEE1, BLK, FER, CDK6, LIMK2, AAK1, CDK5, CDK2, ITK, CDK17, LCK, PTK2B, CDK9, CDK7, CDK13, PRKAA1, CDK12, BMP2K, and STK10, and is represented by structure:

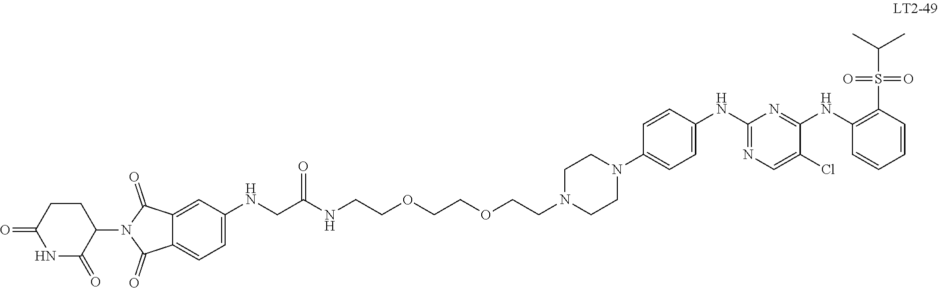 [SMILES: CC(C)S(=O)(=O)c1ccccc1Nc1nc(Nc2ccc(N3CCN(CCOCCOCCNC(=O)CNc4ccc5c(c4)C(=O)N(C4CCC(=O)NC4=O)C5=O)CC3)cc2)ncc1Cl]

or a pharmaceutically acceptable salt or stereoisomer thereof.

In some embodiments, the bifunctional compound degrades ABL2, EPHB2, SIK2, and TYK2, and is represented by structure:

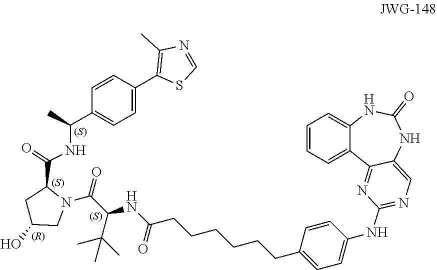

or a pharmaceutically acceptable salt or stereoisomer thereof.

In some embodiments, the bifunctional compound degrades AAK1, CDK16, WEE1, GAK, MARK4, NEK9, RPS6KB1, SIK2, SIK3, SNRK, STK17A, and STK17B, and is represented by structure:

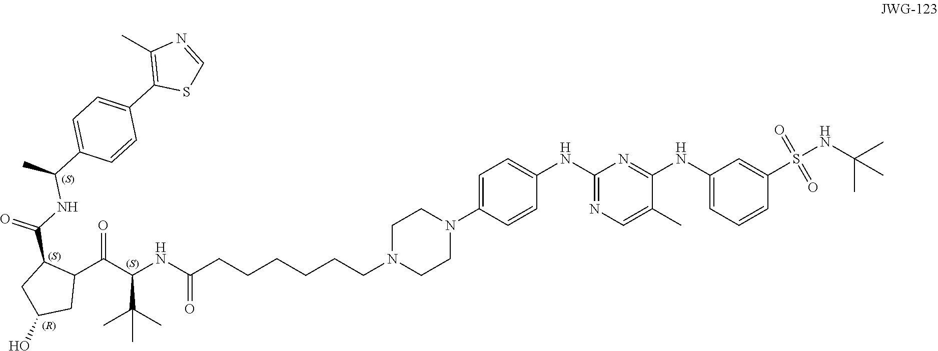

or a pharmaceutically acceptable salt or stereoisomer thereof.

In some embodiments, the bifunctional compound degrades AAK1 and GAK, and is represented by structure:

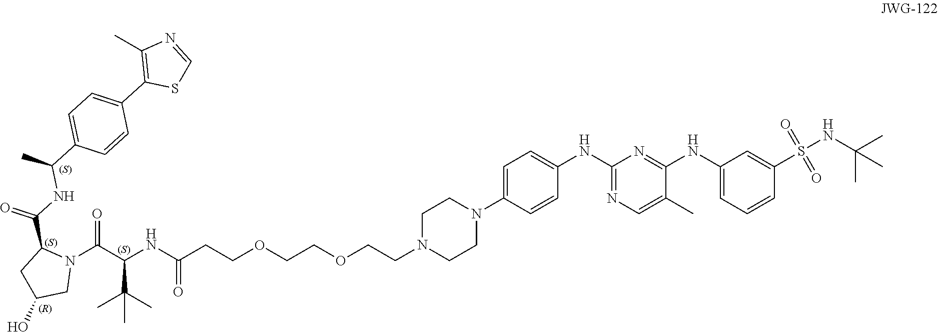

or a pharmaceutically acceptable salt or stereoisomer thereof.

In some embodiments, the bifunctional compound degrades AAK1 and AURKA, and is represented by structure:

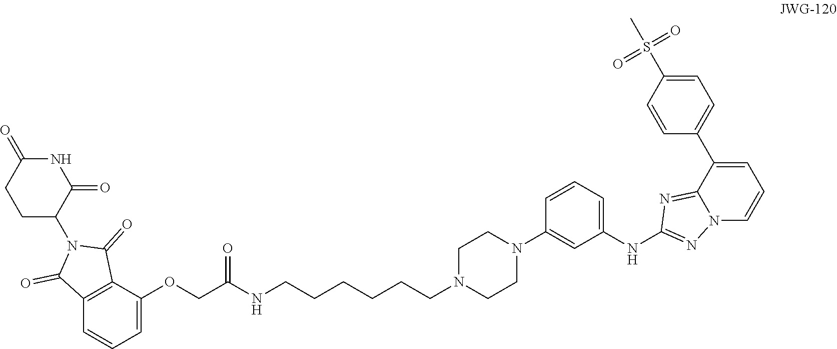 [SMILES: CS(=O)(=O)c1ccc(-c2cccn3nc(Nc4cccc(N5CCN(CCCCCCNC(=O)COc6cccc7c6C(=O)N(C6CCC(=O)NC6=O)C7=O)CC5)c4)nc23)cc1]

or a pharmaceutically acceptable salt or stereoisomer thereof.

In some embodiments, the bifunctional compound degrades AAK1, AURKA, BMP2K, GAK, and WEE1, and is represented by structure:

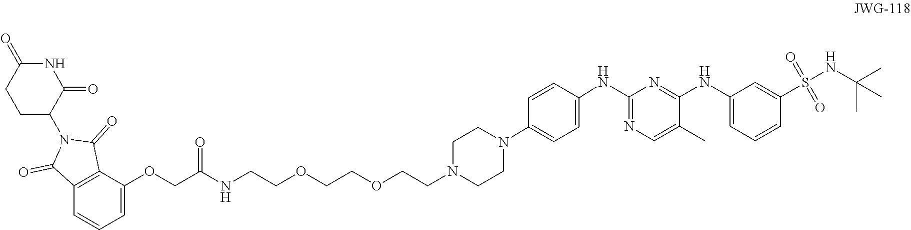 [SMILES: Cc1cnc(Nc2ccc(N3CCN(CCOCCOCCNC(=O)COc4cccc5c4C(=O)N(C4CCC(=O)NC4=O)C5=O)CC3)cc2)nc1Nc1cccc(S(=O)(=O)NC(C)(C)C)c1]

or a pharmaceutically acceptable salt or stereoisomer thereof.

In some embodiments, the bifunctional compound degrades LATS1 and STK17A, and is represented by structure:

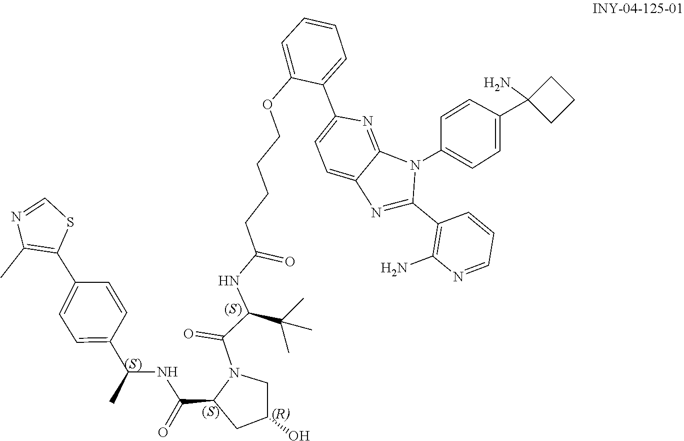

or a pharmaceutically acceptable salt or stereoisomer thereof.

In some embodiments, the bifunctional compound degrades PDK1, PDK2, and PDK3, and is represented by structure:

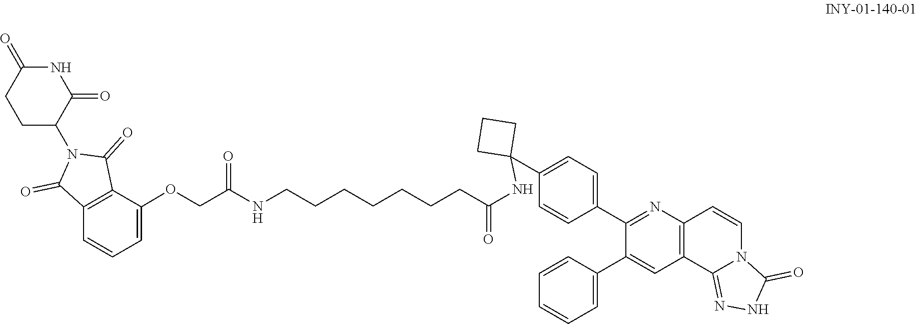 [SMILES: O=C(COc1cccc2c1C(=O)N(C1CCC(=O)NC1=O)C2=O)NCCCCCCCC(=O)NC1(c2ccc(-c3nc4ccn5c(=O)[nH]nc5c4cc3-c3ccccc3)cc2)CCC1]

or a pharmaceutically acceptable salt or stereoisomer thereof.

In some embodiments, the bifunctional compound degrades AAK1, ABL2, AURKA, AURKB, BUB1B, CDC7, CDK1, CDK12, CDK13, CDK2, CDK4, CDk6, CDK7, CDK9, CHEK1, CSNK1D. EPHA1, FER, FGFR1, GAK, IRAK4, ITK, LIMK2, MAP4K2, MAP4K3, MAPK6, MAPK7, MARK4, MELK, PKN3, PLK4, PRKAA1, PTK2, PTK6, RPS6KA4, SIK2, STK35, TNK2. UHMK1, ULK1, and WEE1, and is represented by structure:

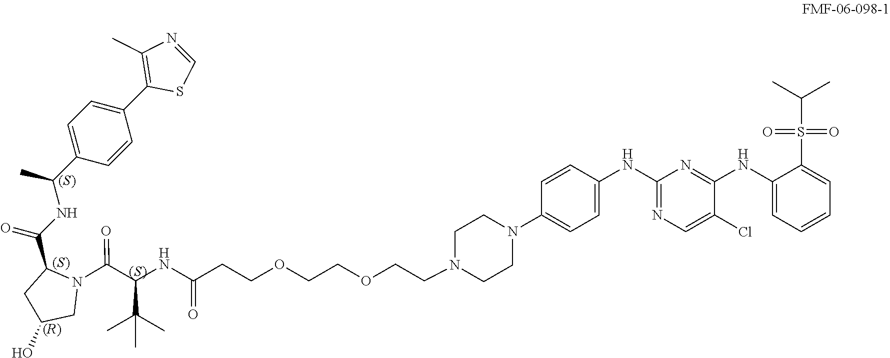

or a pharmaceutically acceptable salt or stereoisomer thereof.

In some embodiments, the bifunctional compound degrades CDK11A, CDK9, CLK1, GSK3A, GSK3B, PIK3CG, and SGK3, and is represented by structure:

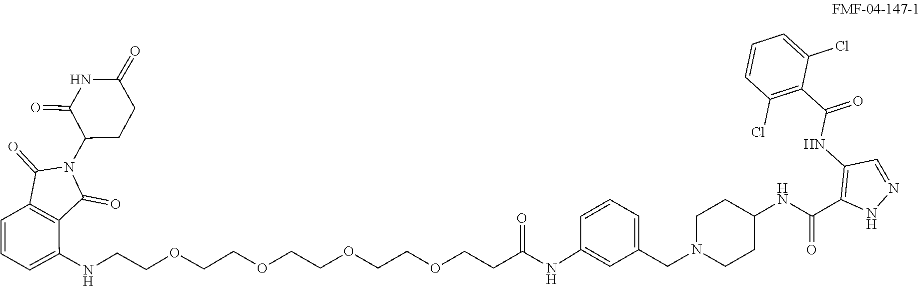 [SMILES: O=C1CCC(N2C(=O)c3cccc(NCCOCCOCCOCCOCCC(=O)Nc4cccc(CN5CCC(NC(=O)c6[nH]ncc6NC(=O)c6c(Cl)cccc6Cl)CC5)c4)c3C2=O)C(=O)N1]

or a pharmaceutically acceptable salt or stereoisomer thereof.

In some embodiments, the bifunctional compound degrades BLK, CSK, LCK, LIMK2, MAP2K5, and MAP3K20, and is represented by structure:

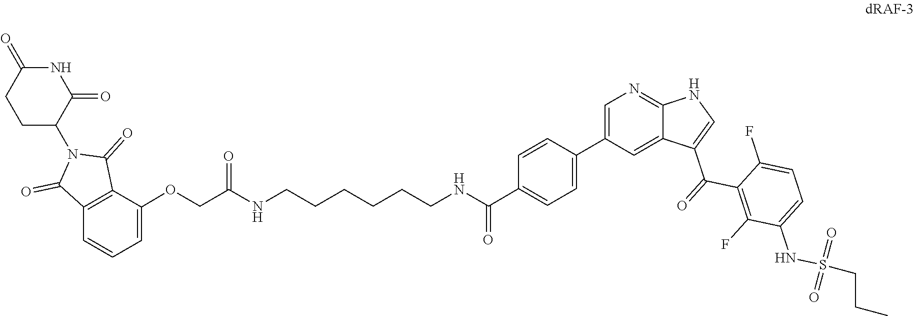 [SMILES: CCCS(=O)(=O)Nc1ccc(F)c(C(=O)c2c[nH]c3ncc(-c4ccc(C(=O)NCCCCCCNC(=O)COc5cccc6c5C(=O)N(C5CCC(=O)NC5=O)C6=O)cc4)cc23)c1F]

or a pharmaceutically acceptable salt or stereoisomer thereof.

In some embodiments, the bifunctional compound degrades CDK17, LIMK1, and LIMK2, and is represented by structure:

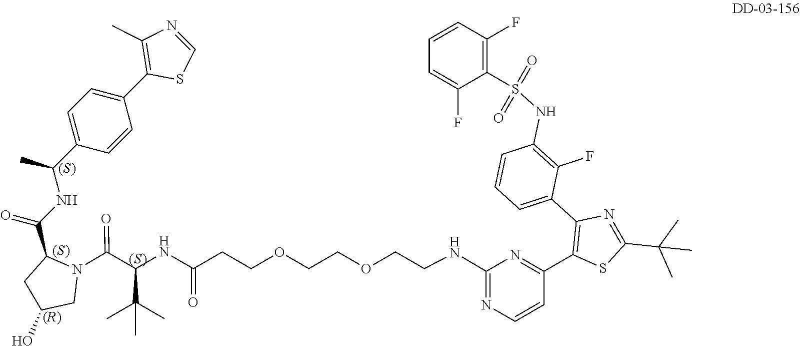

or a pharmaceutically acceptable salt or stereoisomer thereof.

In some embodiments, the bifunctional compound degrades ABL2, BLK, CSK, FYN, LCK, SRC, and TEC, and is represented by structure:

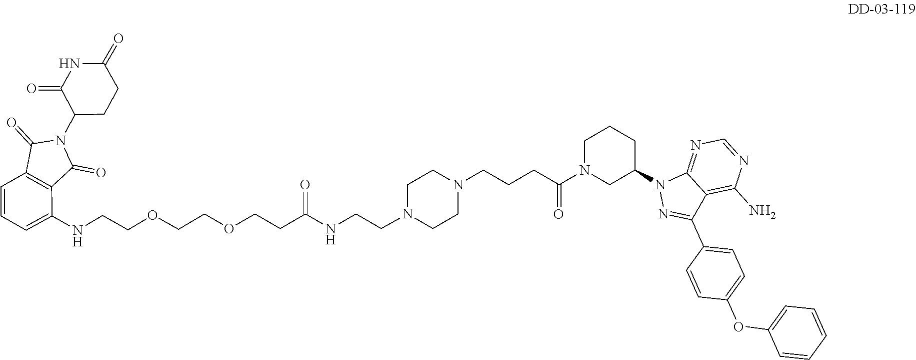 [SMILES: Nc1ncnc2c1c(-c1ccc(Oc3ccccc3)cc1)nn2[C@@H]1CCCN(C(=O)CCCN2CCN(CCNC(=O)CCOCCOCCNc3cccc4c3C(=O)N(C3CCC(=O)NC3=O)C4=O)CC2)C1]

or a pharmaceutically acceptable salt or stereoisomer thereof.

In some embodiments, the bifunctional compound degrades BCKDK, COQ8A, LIMK1, PDK1, PDK2, and PDK3, and is represented by structure:

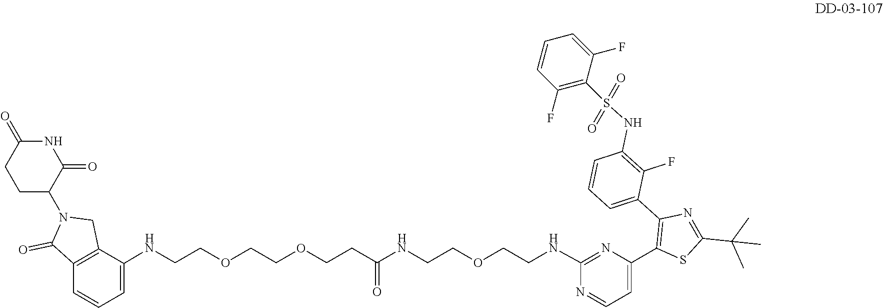 [SMILES: CC(C)(C)c1nc(-c2cccc(NS(=O)(=O)c3c(F)cccc3F)c2F)c(-c2ccnc(NCCOCCNC(=O)CCOCCOCCNc3cccc4c3CN(C3CCC(=O)NC3=O)C4=O)n2)s1]

or a pharmaceutically acceptable salt or stereoisomer thereof.

In some embodiments, the bifunctional compound degrades AURKA, BCKDK, CDK1, CDK16, CDK17, CDK2, CDK3, CDK4, CDK6, COQ8A, COQ8B, CSK, EIF2AK2, LIMK1, LIMK2, MAP3K20, NLK, PLK1, PDK1, PDK2, and TESK2, and is re resented by structure:

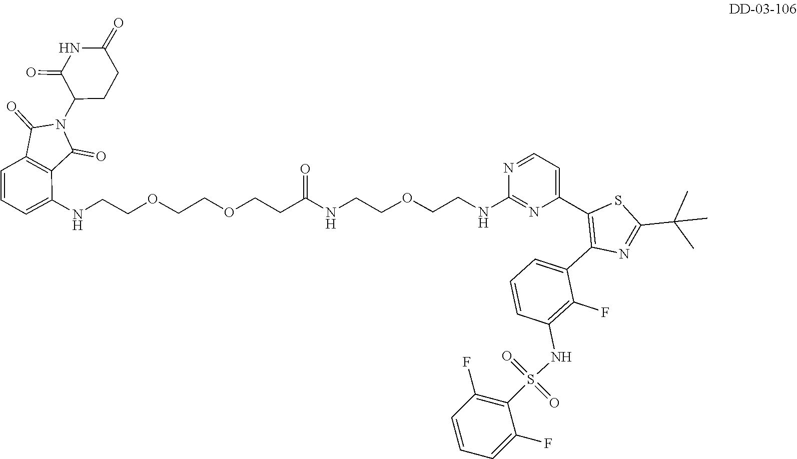 [SMILES: CC(C)(C)c1nc(-c2cccc(NS(=O)(=O)c3c(F)cccc3F)c2F)c(-c2ccnc(NCCOCCNC(=O)CCOCCOCCNc3cccc4c3C(=O)N(C3CCC(=O)NC3=O)C4=O)n2)s1]

or a pharmaceutically acceptable salt or stereoisomer thereof.

In some embodiments, the bifunctional compound degrades MAPK14 and is represented by structure:

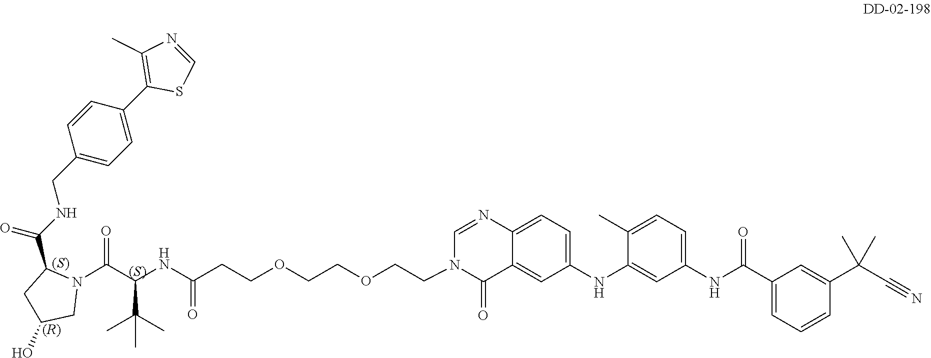

or a pharmaceutically acceptable salt or stereoisomer thereof.

In some embodiments, the bifunctional compound degrades BLK, BUB1, CDK4, LIMK2, SIK2, STK17A, TEC, TNK2, and UHMK1, and is represented by structure:

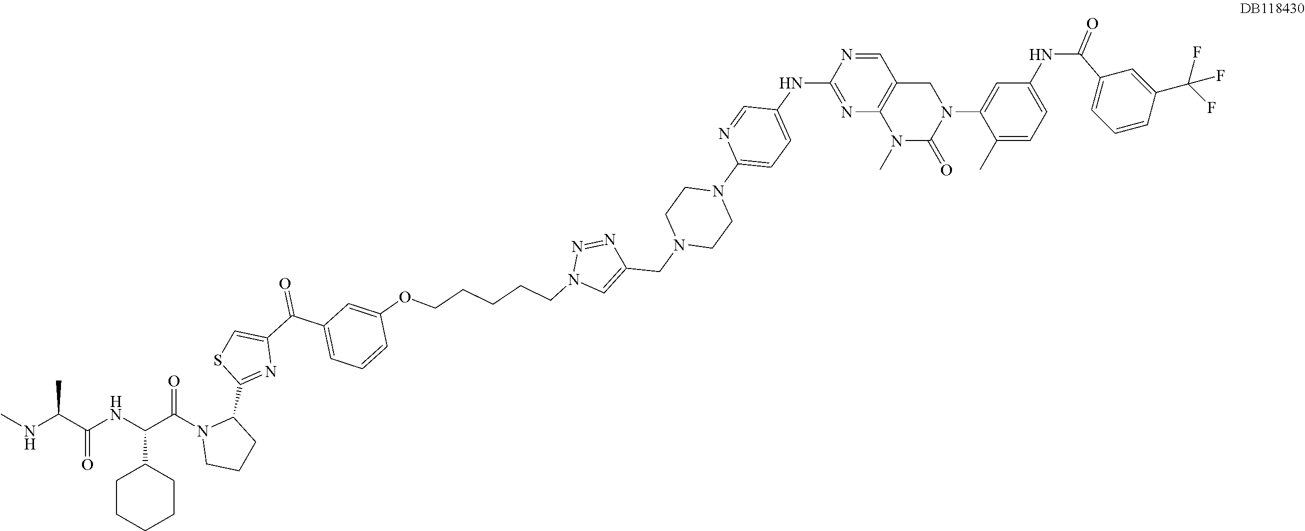 [SMILES: CN[C@@H](C)C(=O)N[C@H](C(=O)N1CCC[C@H]1c1nc(C(=O)c2cccc(OCCCCCn3cc(CN4CCN(c5ccc(Nc6ncc7c(n6)N(C)C(=O)N(c6cc(NC(=O)c8cccc(C(F)(F)F)c8)ccc6C)C7)cn5)CC4)nn3)c2)cs1)C1CCCCC1]

or a pharmaceutically acceptable salt or stereoisomer thereof, 100691In some embodiments, the bifunctional compound degrades ABL1, ABL2, BLK, CDK11B, CDK4, CIT, CSK, EPHA3, FER, GAK, a LCK. LIMK2, MAP3K20, MAP3K7, MAP4K1, MAP4K2, MAP4K5, MAPK14, MAPK7, MAPK9, MAPKAPK2, MAPKAPK3, PDIK1L, PTK2B, RIPK1, RPS6KA1, SIK2, STK35, TAOK2, and ULK1, and is represented by structure:

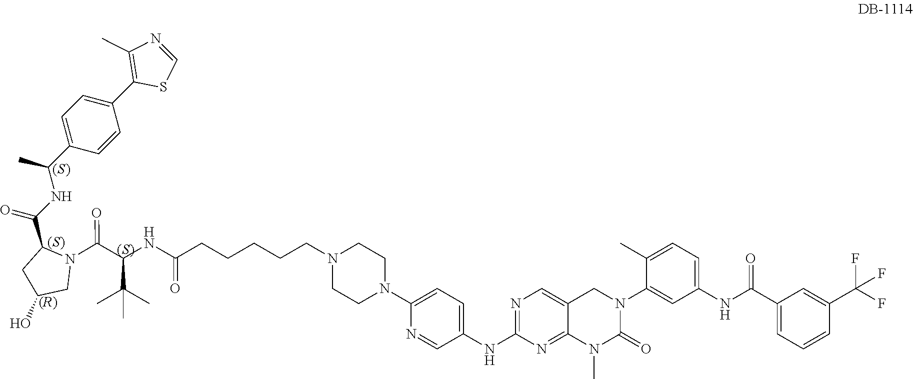

or a pharmaceutically acceptable salt or stereoisomer thereof.

In some embodiments, the bifunctional compound degrades ABL1, ABL2, BLK, CDK11B, CDK4, CSK, EPHA3, FER, GAK, LIMK1, MAP3K20, MAP4K1, MAP4K2, MAP4K3, MAP4K5, MAPK14, MAPK7, MAPK8, MAPK9, MAPKAPK2, MAPKAPK3, NLK, PDIK1L, PTK2B, RIPK1, RPS6KA1, RPS6KA3, SIK2, SIK3, STK35, TNK2, and ULK1, and is represented by structure.

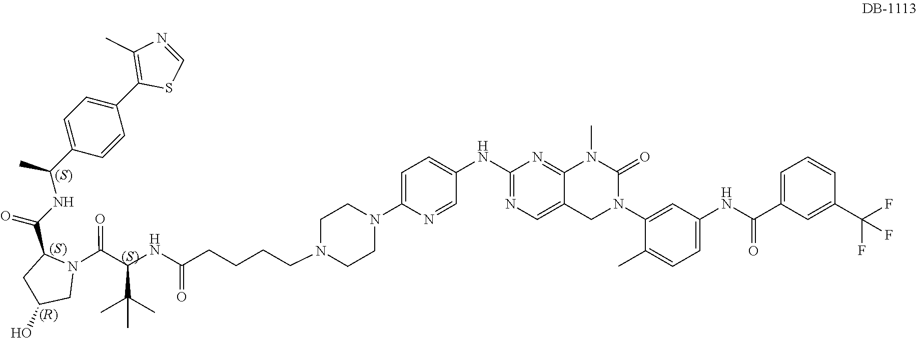

or a pharmaceutically acceptable salt or stereoisomer thereof.

In some embodiments, the bifunctional compound degrades CDK4, BLK, FER, LIMK2, GAK, CSK, SIK2, LCK, PTK2B, SRC, ABL2, MAPK14,a MAPK9, MAP4K2, MKNK2, MAP3K20, and TNK2, and is represented by structure:

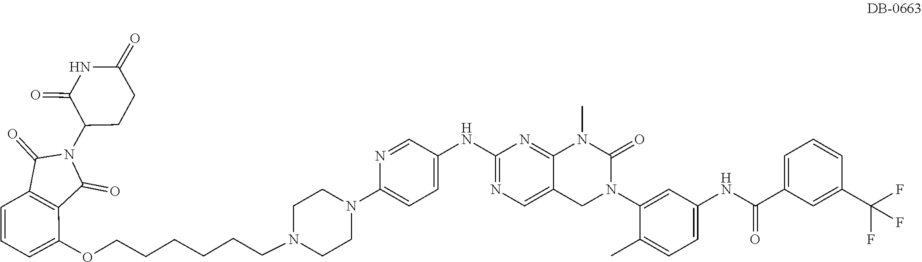 [SMILES: Cc1ccc(NC(=O)c2cccc(C(F)(F)F)c2)cc1N1Cc2cnc(Nc3ccc(N4CCN(CCCCCCOc5cccc6c5C(=O)N(C5CCC(=O)NC5=O)C6=O)CC4)nc3)nc2N(C)C1=O]

or a pharmaceutically acceptable salt or stereoisomer thereof.

In some embodiments, the bifunctional compound degrades ABL1, ABL2, BLK, BUB1, CDK11B, CDK4, CSK, EPHB6, FER. FYN, GAK, LCK, LIMK1, MAP3K1, MAP3K11, MAP3K20, MAP4K1, MAPK14, MAPK8, MAPK9, MAPKAPK2, MKNK2, PAK4, PDIK1L, PTK2B, RPS6KA1, RPS6KA3, SIK2, SRC, TNK2, UHMK1, ULK1, and YES1, and is represented by structure:

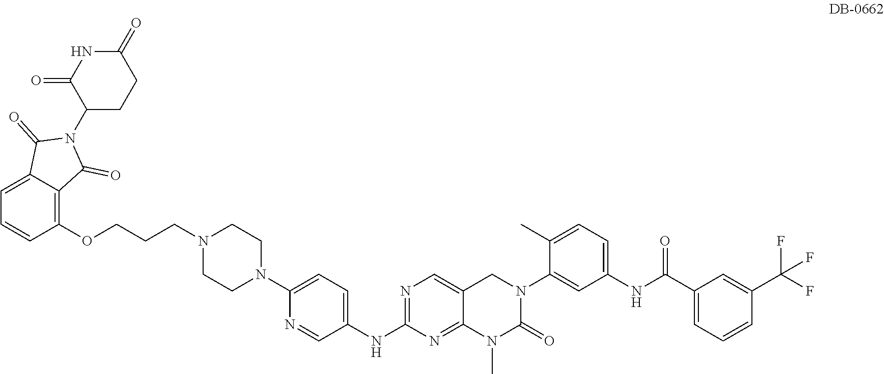 [SMILES: Cc1ccc(NC(=O)c2cccc(C(F)(F)F)c2)cc1N1Cc2cnc(Nc3ccc(N4CCN(CCCOc5cccc6c5C(=O)N(C5CCC(=O)NC5=O)C6=O)CC4)nc3)nc2N(C)C1=O]

or a pharmaceutically acceptable salt or stereoisomer thereof.

In some embodiments, the bifunctional compound degrades BLK, CDK4, CLK1, CSK, FER, LCK, LIMK1, MAPK8, MAPK9, MKNK2, PLK1, PTK2B, SIKA2, SRC, TNK2, UHMK1, and YES1, and is represented by structure:

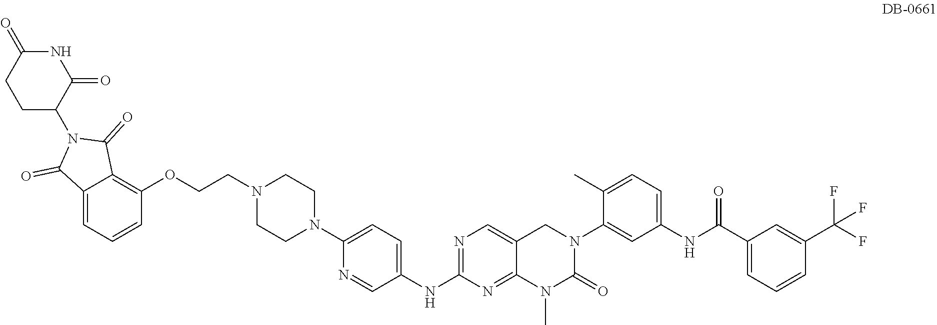 [SMILES: Cc1ccc(NC(=O)c2cccc(C(F)(F)F)c2)cc1N1Cc2cnc(Nc3ccc(N4CCN(CCOc5cccc6c5C(=O)N(C5CCC(=O)NC5=O)C6=O)CC4)nc3)nc2N(C)C1=O]

or a pharmaceutically acceptable salt or stereoisomer thereof.

In some embodiments, the bifunctional compound degrades ABL2, AURKA, BLK, BUB1, CDK11A, CDK11B, CDK4, CSK, DDR2, EPHA3, EPHB3, EPHB6, FER, FYN, GAK. LATS1, LCK, LIMK1, LIMK2, LRRK2, LYN, MAP3K1, MAP3K11. MAP3K20, MAP4K1, MAP4K2, MAP4K5, MAPK11, MAPK12, MAPK14, MAPK8, MAPK9, MAPKAPK2, MKNK2, NLK, PLK1, PTK2, PTK2B, RIPK1, RIPK2, RPS6KA3, SIK2, SRC, TAOK2, TEC, TNK2, TTK, UHMK1, ULK1, WEE1, and YES1, and is represented by structure:

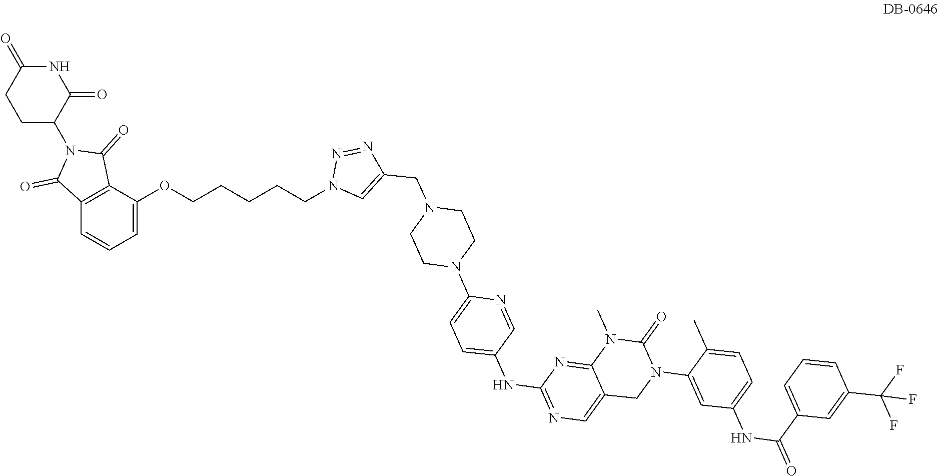 [SMILES: Cc1ccc(NC(=O)c2cccc(C(F)(F)F)c2)cc1N1Cc2cnc(Nc3ccc(N4CCN(Cc5cn(CCCCCOc6cccc7c6C(=O)N(C6CCC(=O)NC6=O)C7=O)nn5)CC4)nc3)nc2N(C)C1=O]

or a pharmaceutically acceptable salt or stereoisomer thereof.

In some embodiments, the bifunctional compound degrades AAK1, AURKA, BMP2K, CAMKK1, CDK16, CDK4, CDK6, EIF2AK2, FER, GAK, LCK, LIMK2, MAP3K11, MAPK8, MAPK9, NEK9, PLK4, PTK2B, SIK2, STK17A, STK17B, ULK1, ULK3, and WEE1, and is represented by structure:

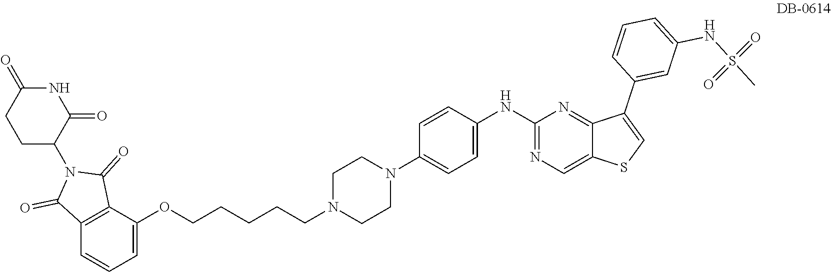 [SMILES: CS(=O)(=O)Nc1cccc(-c2csc3cnc(Nc4ccc(N5CCN(CCCCCOc6cccc7c6C(=O)N(C6CCC(=O)NC6=O)C7=O)CC5)cc4)nc23)c1]

or a pharmaceutically acceptable salt or stereoisomer thereof.

In some embodiments, the bifunctional compound degrades AURKA and AURKB, and is represented by structure:

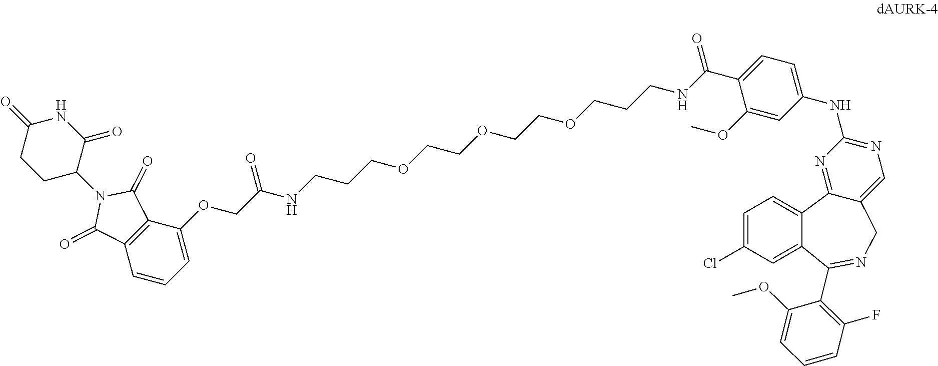 [SMILES: COc1cc(Nc2ncc3c(n2)-c2ccc(Cl)cc2C(c2c(F)cccc2OC)=NC3)ccc1C(=O)NCCCOCCOCCOCCCNC(=O)COc1cccc2c1C(=O)N(C1CCC(=O)NC1=O)C2=O]

or a pharmaceutically acceptable salt or stereoisomer thereof.

In some embodiments, the bifunctional compound degrades AAK1, GAK, MARK2, MARK3, MARK4, RPS6KB1. SIK2, SIK3, SNRK, STK17A, STK17B, ULK1, and WEE1, and is represented by structure:

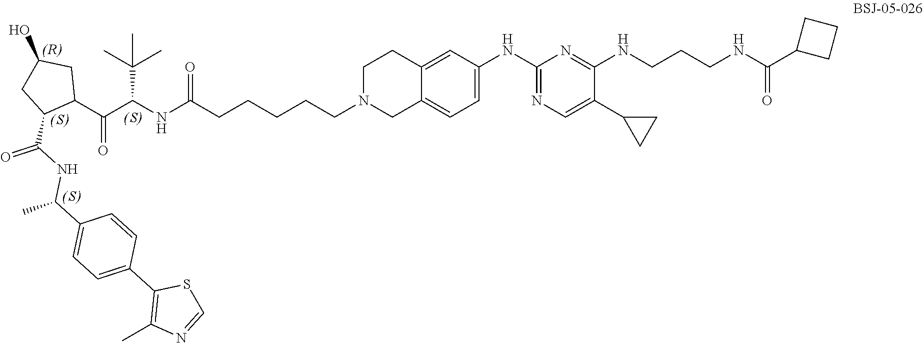

or a pharmaceutically acceptable salt or stereoisomer thereof.

In some embodiments, the bifunctional compound degrades AAK1, AURKA, AURKB. BMP2K, CDK10, CDK9, GAK, MARK2, MARK3, MARK4, SIK2, STK17A, STK17B, SNRK, ULK1, and WEE1, and is represented by structure:

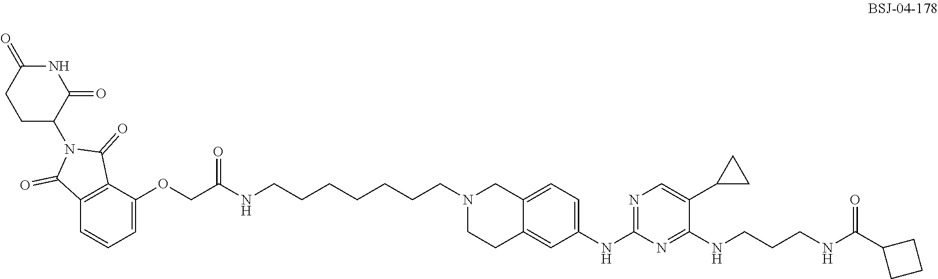 [SMILES: O=C(COc1cccc2c1C(=O)N(C1CCC(=O)NC1=O)C2=O)NCCCCCCCN1CCc2cc(Nc3ncc(C4CC4)c(NCCCNC(=O)C4CCC4)n3)ccc2C1]

or a pharmaceutically acceptable salt or stereoisomer thereof.

In some embodiments, the bifunctional compound degrades AAK1, AURKA, AURKB, BMP2K, CDK9, EPHB2, GSK3B, ITK, LATS1, MAP4K2, NEK9, PAK4, PLK4, and STK L7B, and is represented by structure:

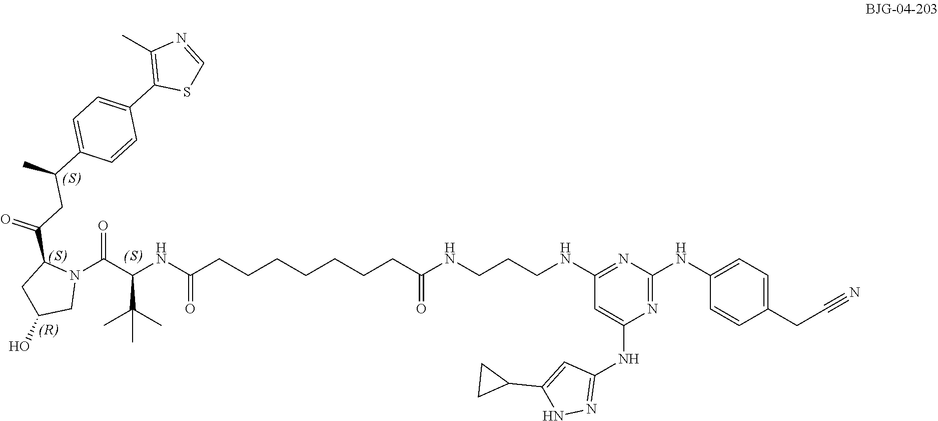

or a pharmaceutically acceptable salt or stereoisomer thereof.

In some embodiments, the bifunctional compound degrades ABL1, ABL2, AURKA, BLK, CSK, EPHA3, EPHB6, FYN, GAK, LCK. LIMK2, MAPK14, NLK, PDK1, PKMYT1, SIK2, SRC, TNK2, WEE1, and YES1, and is represented by structure:

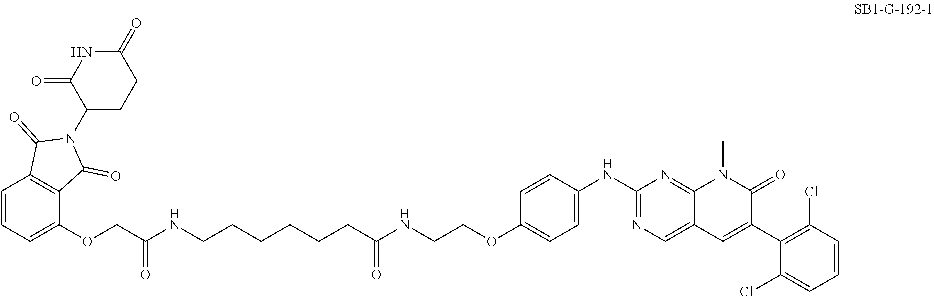 [SMILES: Cn1c(=O)c(-c2c(Cl)cccc2Cl)cc2cnc(Nc3ccc(OCCNC(=O)CCCCCCNC(=O)COc4cccc5c4C(=O)N(C4CCC(=O)NC4=O)C5=O)cc3)nc21]

or a pharmaceutically acceptable salt or stereoisomer thereof.

In some embodiments, the bifunctional compound degrades ABL2, BLK, CSK, and WEE1, and is represented by structure:

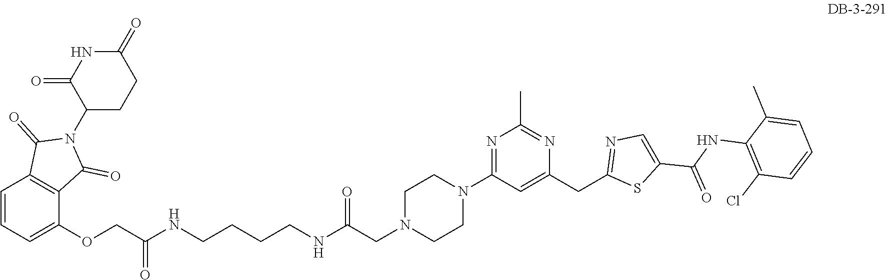 [SMILES: Cc1nc(Cc2ncc(C(=O)Nc3c(C)cccc3Cl)s2)cc(N2CCN(CC(=O)NCCCCNC(=O)COc3cccc4c3C(=O)N(C3CCC(=O)NC3=O)C4=O)CC2)n1]

or a pharmaceutically acceptable salt or stereoisomer thereof.

Bifunctional compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

Bifunctional compounds of the present invention may have at least one chiral center. Therefore, they may be in the form of a stereoisomer. As used herein, the term “stereoisomer” embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the bifunctional compound of the present invention is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium. i.e. ²H, may afford certain therapeutic advantages resulting from greater metabolic stability, for example, increased in vivo half-life or reduced dosage requirements, and thus may be advantageous in some circumstances.

In addition, bifunctional compounds of the present invention embrace N-oxides, crystalline forms (also known as polymorphs), active metabolites of the compounds having the same type of activity, tautomers, and unsolvated as well as solvated and hydrated forms with pharmaceutically acceptable solvents such as water, ethanol, and the like, of the compounds. The solvated forms of the conjugates presented herein are also considered to be disclosed herein.

### Methods of Synthesis

In some embodiments, the present invention is directed to a method for making a bifunctional compounds the present invention or a pharmaceutically acceptable salts or stereoisomers thereof. Broadly, the inventive compounds or pharmaceutically-acceptable salts or stereoisomers thereof, may be prepared by any process known to be applicable to the preparation of chemically related compounds. The compounds of the present invention will be better understood in connection with the synthetic schemes that described in various working examples that illustrate non-limiting methods by which the compounds of the invention may be prepared.

### Pharmaceutical Compositions

Another aspect of the present invention is directed to a pharmaceutical composition that includes a therapeutically effective amount of a bifunctional compound of the present invention or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may further include one or more pharmaceutically acceptable excipients.

Broadly, bifunctional compounds of the present invention and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of the present invention may be formulated in a hard or soft gelatin capsule. Representative excipients that may be used include 44egelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of the present invention may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bifunctional compounds may be formulated for buccal or sublingual administration, examples of which include tablets, lozenges and gels.

The bifunctional compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of the present invention may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I, and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound of the present invention or a pharmaceutically acceptable salt or a stereoisomer thereof, or a composition including a bifunctional compound of the present invention or a pharmaceutically acceptable salt or a stereoisomer thereof, effective in producing the desired therapeutic response in a particular patient suffering from a disease or disorder characterized or mediated by aberrant protein activity. The term “therapeutically effective amount” thus includes the amount of a bifunctional compound of the invention or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amount of aberrant proteins in diseased cells.

The total daily dosage of the bifunctional compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof(e.g., its present status); the age, body weight, general health, sex and diet of the subject; the time of administration, route of administration, and rate of excretion of the specific compound employed; the duration of the treatment; drugs used in combination or coincidental with the bifunctional compound; and like factors well known in the medical arts (see, for example, Goodman and Gilman's The Pharmacological Basis of Therapeutics, 10th Edition, A. Gilman, J. Hardman and L. Limbird, eds., McGraw-Hill Press, 155-173, 2001).

Bifunctional compounds of the present invention and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a bifunctional compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present invention is directed to methods of treating diseases or disorders involving aberrant protein activity, that entails administration of a therapeutically effective amount of a bifunctional compound of the present invention or a pharmaceutically acceptable salt or stereoisomer thereof, to a subject in need thereof.

The diseases or disorders are characterized or mediated by aberrant protein activity (e.g., elevated levels of the protein or otherwise functionally abnormal the protein relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

In some embodiments, bifunctional compounds of the present invention may be used to treat diseases or disorders involving aberrant AP2-associated protein kinase 1 (AAK1), ABL proto-oncogene (ABL)1, ABL2, Serine/Threonine kinase (AKT)2, AKT3, Aurora kinase (AURK)4, AURKA, AURKB, branched chain ketoacid dehydrogenase kinase (BCKDK), B-lymphoid tyrosine kinase (BLK), BMP-2-inducible protein kinase (BMP2K), Bone morphogenetic protein receptor type-1A (BMPR1A), mitotic checkpoint serine/threonine-protein kinase BUB 1 (BUB1), BUB1B, calcium/calmodulin-dependent protein kinase kinase 1 (CAMKK1), cell division cycle 7 (CDC7), cyclin-dependent kinase (CDK)1, CDK1O, CDK11A, CDK11B, CDK12, CDK13, CDK14, CDK16, CDK17, CDK18, CDK2, CDK3, CDK4, CDK5, CDK6, CDK7, CDK9, Checkpoint kinase 1(CHEK1), citron Rho-interacting kinase (CIT), CDC Like Kinase 1 (CLK1), coenzyme Q8 (COQ8)A, COQ8B, Tyrosine-protein kinase CSK (CSK), casein kinase 1 (CSNK1)A1, CSNK1D, CSNK1E, death-associated protein kinase 1 (DAPK1), discoidin domain-containing receptor 2 (DDR2), eukaryotic translation initiation factor 2-alpha kinase (EIF2AK)2, EIF2AK4, ephrin type-A receptor (EPHA)l, EPHA2, EPHA3, ephrin type-B receptor (EPHB)2, EPHB3, EPHB4, EPHB6, endoplasmic reticulum to nucleus signaling 1 (ERN1), tyrosine-protein kinase Fer (FER), fibroblast growth factor receptor 1 (FGFR1), fibroblast growth factor receptor 2 (FGR2), proto-oncogene tyrosine-protein kinase Fyn (FYN), cyclin G-associated kinase (GAK), glycogen synthase kinase 3 (GSK3)A, GSK3B, homeodomain-interacting protein kinase 1 (HIPK1), interleukin-1 receptor-associated kinase (IRAK)1, IRAK4, tyrosine-protein kinase ITK/TSK (ITK), large tumor suppressor kinase 1 (LATS1), lymphocyte cell-specific protein-tyrosine kinase (LCK), LIM domain kinase (LIMK)1, LIMK2, leucine-rich repeat kinase 2 (LRRK2), tyrosine-protein kinase Lyn (LYN), dual specificity mitogen-activated protein kinase kinase 5 (MAP2K5), mitogen-activated protein kinase kinase kinase (MAP3K)1, MAP3K11. MAP3K12, MAP3K20, MAP3K21, MAP3K7, mitogen-activated protein kinase kinase kinase kinase (MAP4K)1, MAP4K2, MAP4K3, MAP4K5, mitogen-activated protein kinase (MAPK)11, MAPK12, MAPK14, MAPK6, MAPK7, MAPK8, MAPK9, mitogen-activated protein kinase-activated protein kinase (MAPKAPK)2, MAPKAPK3, MAPKAPK5, microtubule affinity regulating kinase (MARK)2, MARK3, MARK4, microtubule-associated serine/threonine-protein kinase 3 (MAST3), maternal embryonic leucine zipper kinase (MELK), misshapen like kinase 1 (MINK1), MAP kinase-interacting serine/threonine-protein kinase 2 (MKNK2), never in mitosis A-related kinase (NEK)2, NEK9, nemo like kinase (NLK). NUAK family SNF1-like kinase 1 (NUAK1), serine/threonine-protein kinase PAK 4 (PAK4), serine/threonine-protein kinase PDIK1L (PDIK1L), 3-phosphoinositide-dependent protein kinase (PDK)1, PDK2, PDK3, phosphatidylinositol 4,5-bisphosphate 3-kinase catalytic subunit gamma isoform (PIK3CG), serine/threonine-protein kinase pim-2 (PIM2), membrane-associated tyrosine- and threonine-specific cdc2-inhibitory kinase (PKMYT1), serine/threonine-protein kinase N3 (PKN3), polo like kinase (PLK)1, PLK4, PEAK1 related, kinase-activating pseudokinase 1 (PRAG1), 5-AMP-activated protein kinase catalytic subunit alpha (PRKAA)1, PRKAA2, protein tyrosine kinase (PTK)2. PTK2B, PTK6, RIO kinase 2 (RIOK2), receptor-interacting serine/threonine-protein kinase (RIPK)1, RIPK2, ribosomal protein S6 kinase 2 alpha (RPS6KA)1, RPS6KA3, RPS6KA4, RPS6KA6, ribosomal protein S6 kinase beta 1 (RPS6KB1), ribosomal protein S6 kinase beta C1 (RPS6KC1), SH3 domain binding kinase 1 (SBK1), serum/glucocorticoid-regulated kinase 3 (SGK3), salt inducible kinase (SIK)2, SIK3, SIKA2, sucrose nonfermenting 1-related kinase (SNRK), proto-oncogene tyrosine-protein kinase Src (SRC), serine/threonine-protein kinase (STK)10, STK17A, STK17B, STK32C, STK33, STK35, STK38, STK4, STK40, thousand and one amino-acid kinase (TAOK)2. TAOK3, tyrosine-protein kinase Tec (TEC), dual specificity testis-specific protein kinase 2 (TESK2), transforming growth factor beta receptor 1 (TGFBR1), tyrosine kinase non receptor (TNK)1, TNK2, Tribbles homolog 3 (TRIB3), transient receptor potential cation channel subfamily M member 7 (TRPM7), dual specificity protein kinase TTK (TTK), non-receptor tyrosine-protein kinase (TYK2) TYK2, U2AF homology motif kinase 1 (UHMK1), unc-51 like autophagy activating kinase (ULK)1, ULK3, WEE1 G2 checkpoint kinase (WEE1), or YES proto-oncogene 1 (YES1).

The term “subject” (or “patient”) as used herein includes all members of the animal kingdom prone to or suffering from the indicated disease or disorder. In some embodiments, the subject is a mammal. e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In some embodiments, bifunctional compounds of the present invention may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, infectious diseases (e.g., viral diseases), chronic and acute kidney diseases or injuries, metabolic diseases, and allergic and genetic diseases.

In some embodiments, the bifunctional compounds may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD), Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the bifunctional compounds may be useful in the treatment of autoimmune diseases and disorders. As used herein, the term “autoimmune disease” refers to conditions where the immune system produces antibodies that attack normal body tissues. Representative examples of such diseases include autoimmune hematological disorders (e.g., hemolytic anemia, aplastic anemia, anhidrotic ectodermal dysplasia, pure red cell anemia and idiopathic thrombocytopenia), Sjogren's syndrome. Hashimoto thyroiditis, rheumatoid arthritis, juvenile (type 1) diabetes, polymyositis, scleroderma. Addison's disease, lupus including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy. Wegener granulomatosis, autoimmune oophoritis, sarcoidosis, rheumatic carditis, ankylosing spondylitis, Grave's disease, autoimmune thrombocytopenic purpura, psoriasis, psoriatic arthritis, dermatitis herpetiformis, ulcerative colitis, and temporal arteritis.

In other embodiments, the methods are directed to treating subjects having cancer. Broadly, the bifunctional compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eve cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinemia, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the bifunctional compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present invention entail treatment of subjects having cell proliferative diseases or disorders of the hematological system, liver, brain, lung, colon, pancreas, prostate, ovary, breast, skin and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a bifunctional compound of the present invention may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis. MYH associated polyposis, Gardner's syndrome, Peutz-Jeghers syndrome. Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

The bifunctional compounds of the present invention may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments, or “second-line”, as a treatment in patients who have undergone a prior anti-cancer treatment regimen, either alone or in combination with other treatments: or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment. i.e., to prevent reoccurrence of cancer in patients with no currently detectable disease or after surgical removal of a tumor. Thus, in some embodiments, the bifunctional compounds may be administered to a patient who has received another therapy, such as chemotherapy, radioimmunotherapy, surgical therapy, immunotherapy, radiation therapy, targeted therapy or any combination thereof.

The methods of the present invention may entail administration of bifunctional compounds of the present invention or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of five days.

In some aspects, the compounds of the present invention may be useful tools for rapidly interrogating targeted protein degradation of a plurality of kinases.

### Combination Therapy

Bifunctional compounds of the present invention may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bifunctional compound of the present invention in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference. 60th ed., 2006. For example, anti-cancer agents that may be suitable for use in combination with the inventive bifunctional compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes. HDAC inhibitors, proteasome inhibitors, and topoisomerase inhibitors), immunomodulators, therapeutic antibodies (e.g., mono-specific and bifunctional antibodies) and CAR-T therapy.

In some embodiments, the bifunctional compound of the present invention and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 1I hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, the bifunctional compound of the present invention and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present bifunctional compounds and/or compositions containing them may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a bifunctional compound of the present invention or a pharmaceutical composition thereof. The kits or pharmaceutical systems of the invention may also include printed instructions for using the compounds and compositions.

### Methods for Identifying Degradable Kinases

A further aspect of the present invention is directed to methods for identifying a degradable kinase comprising:
assembling a kinase-targeting degrader library comprising a plurality of kinase-targeting scaffolds;
prescreening candidate degrader compounds for cellular permeability in a relevant E3-ligase target engagement assay;
selecting a cell permeable degrader for further characterization of degradation targets;
treating a cell with the selected cell permeable degrader;
employing whole cell multiplexed quantitative proteomics to measure changes in abundance of the proteome in response to treatment with the degrader relative to DMSO; and analyzing the generated datasets to calculate kinase degradation frequency across the library, as a measure of target tractability.

In some embodiments, the degradation targets are further characterized using unbiased mass-spectrometry-based global proteomics analysis, based on chemical diversity and ranking in cellular ligase engagement assays relative to close analogs.

In some embodiments, the relevant E3-ligase target engagement assay is a cereblon (CRBN) or Von Hippel-Lindau tumor suppressor (VHL) target engagement assay.

In some embodiments, the cell is ar mammalian cell. In some embodiments, the mammalian cell is a human cell.

In some embodiments, the cell is a myeloid cell, lymphoid cell, neural cell, epithelial cell, endothelial cell, stem or progenitor cell, hepatocyte, myoblast, osteoblast, osteoclast, lymphocyte, keratinocyte, melanocyte, mesothelial cell, germ cell, muscle cell, fibroblast, transformed cell, or cancer cell.

In some embodiments, the cell is a HEK293T, MOLT-4, Mino, MM1.S, OVCAR-8, KATO III, or KELLY cell.

In some embodiments, the cell is treated with a cell permeable degrader for 1 h, 2 h, 3 h, 4 h, 5 h, 6 h, 7 h, or 8 h.

In some embodiments, the cell is treated with a cell permeable degrader for 5 h.

In some embodiments, the cell is treated with 0.1-10 μM cell permeable degrader.

In some embodiments, the cell is treated with 0.1-5 μM cell permeable degrader.

In some embodiments, the cell is treated with 1 μM cell permeable degrader.

In some embodiments, the abundance fold change cutoff is set at −1.25, and P-value<0.01.

In some embodiments, the methods may also be used for rapidly identifying optimal kinase:scaffold pairs.

A comprehensive experimental map of the degradable kinome was build using the methods described herein. A library of 91 kinase-targeting degrader molecules designed to target all clades of the kinome was used to establish meta-data guided principles for degrader design. In addition, chemical starting points for more than 200 distinct kinases are reported. Through analysis of this unprecedented dataset fundamental rules of induced protein degradation were formulated.

Previous studies have alluded to various development guidelines that make sense given their respective few targets, but oftentimes contradict the guidelines of another study. The study described herein provides a sufficient dataset to be able to 1) definitively say (with broad supporting data) that degrader development is an empirical process and 2) invalidate some of the pet hypotheses from the field.

A key benefit of having large datasets is to provide chemical starting points to skip the guess-make-test steps of development. Built from a kinase degrader library of 91 molecules and tested across 156 independent proteomics treatments, the database described herein enabled a systematic discovery of unexpected leads for degraders, as exemplified below. The methods of the present invention provide an efficient screening approach that presents a wealth of starting points for further medicinal chemistry-based optimization, allowing researchers to rapidly hone in on the most promising path for degrader development for a target of interest, reducing the amount of trial-and-error in the discovery phase.

### Mapping the Degradable Kinome

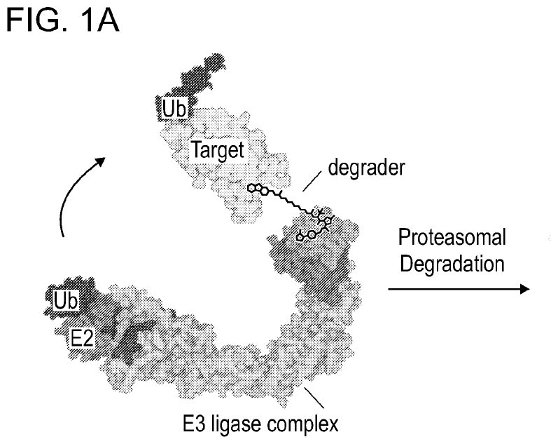

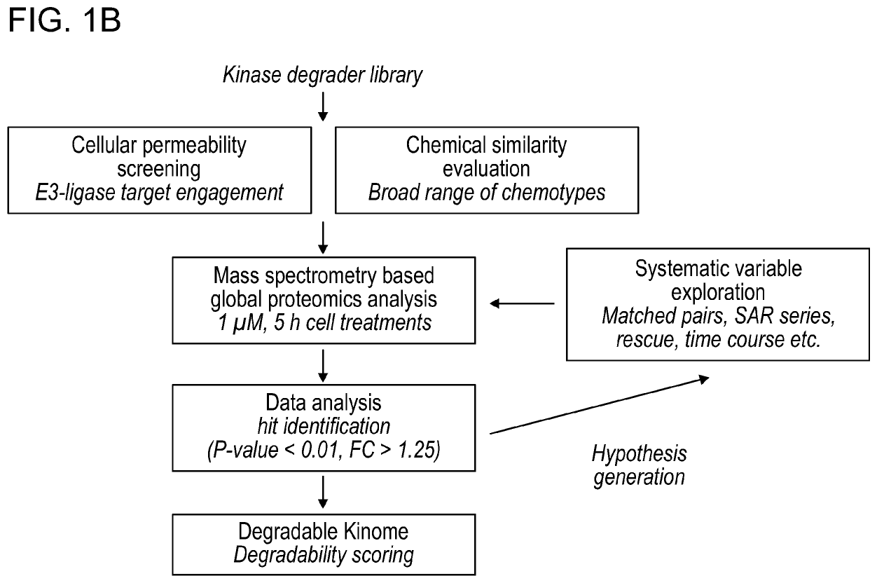

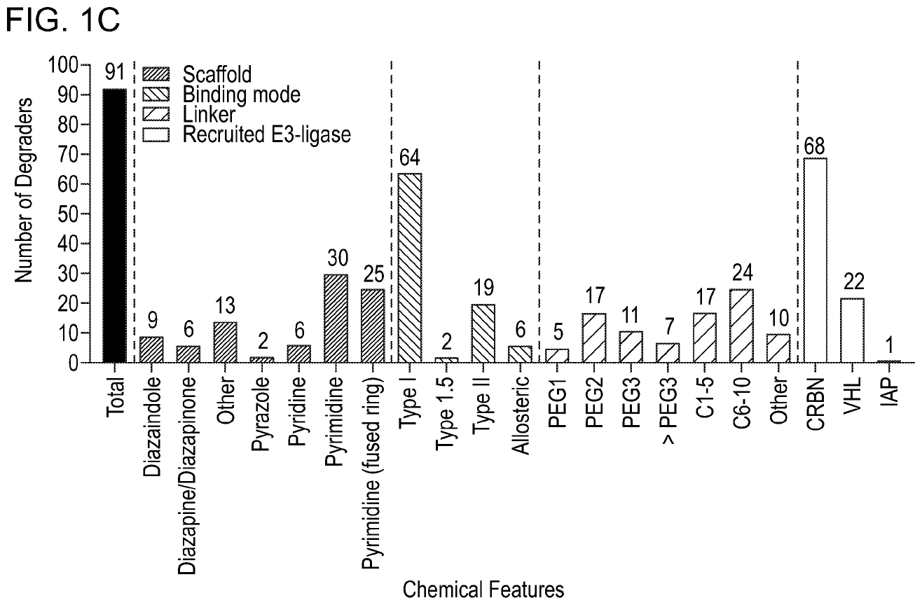

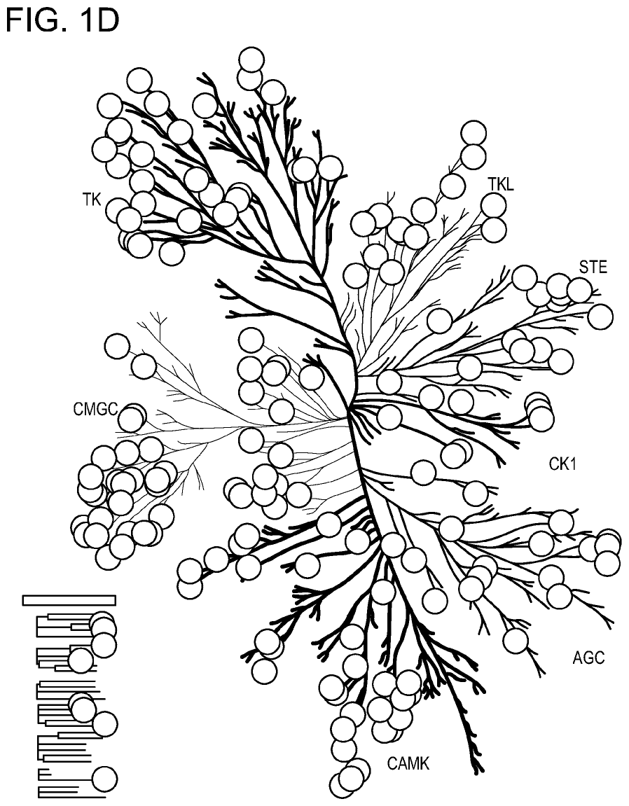

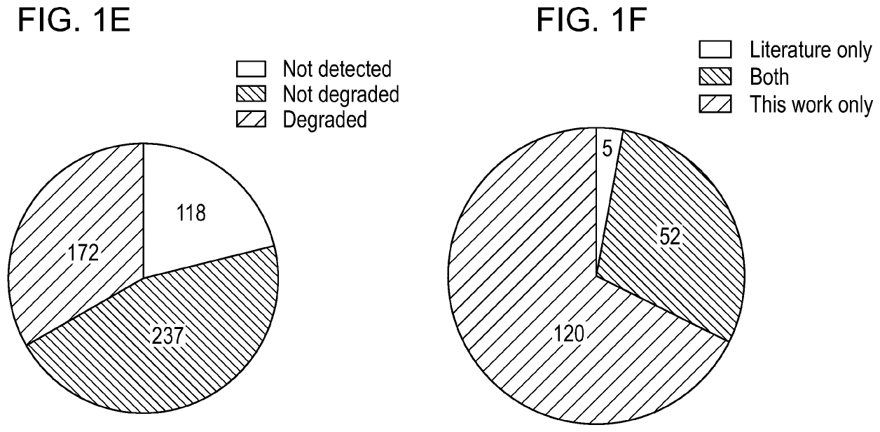

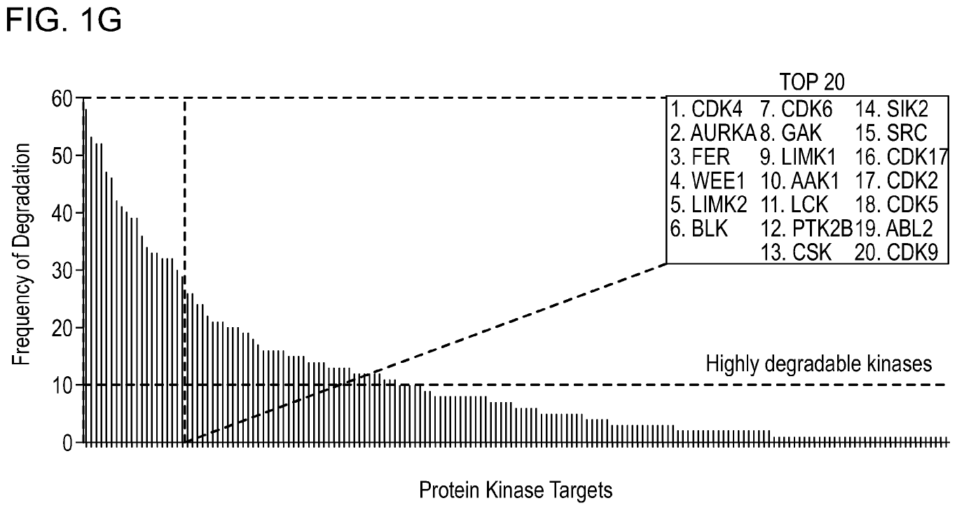

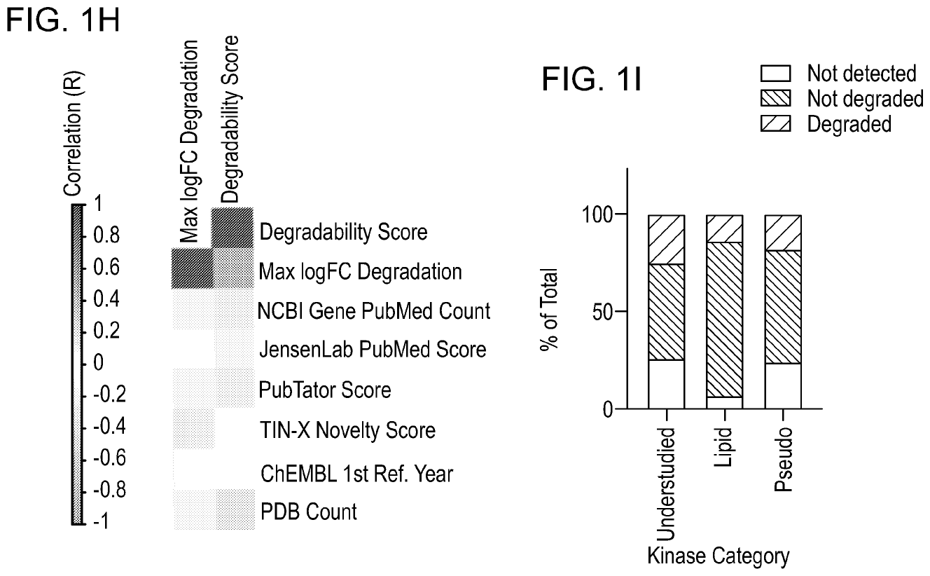

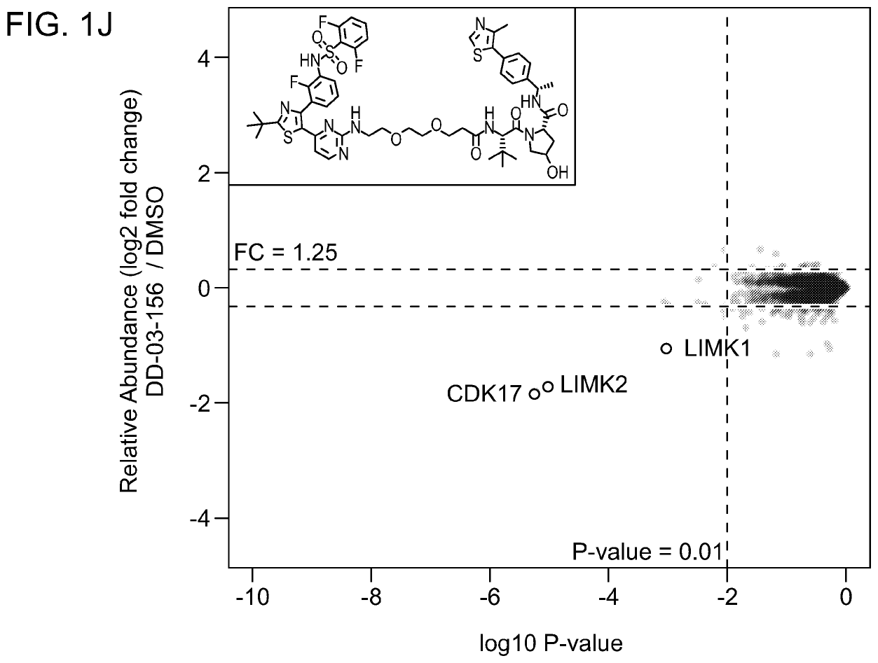

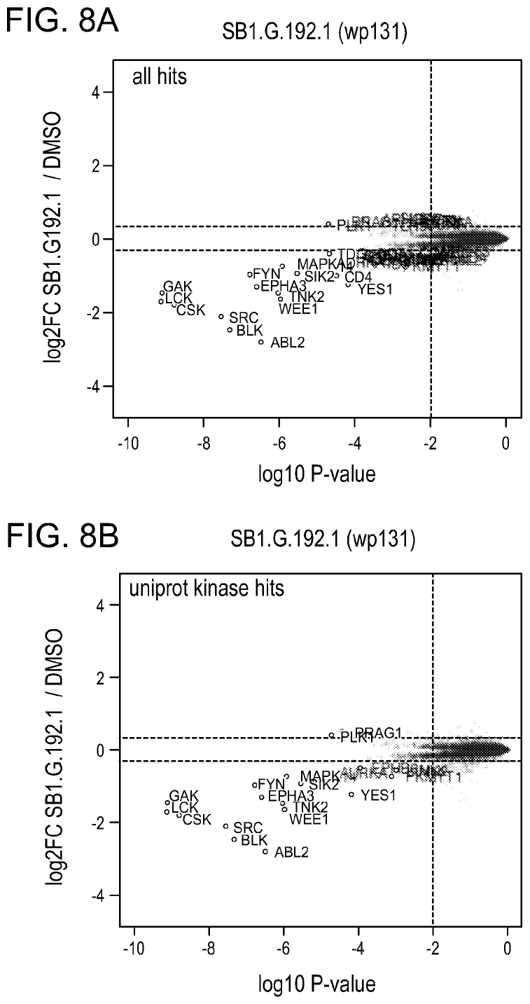

The human protein kinase super family consists of 514 protein kinases (Manning et al., Science 298:1912-1934 (2002)), which makes up 2.5% of the total human genome. Utilizing the vast amount of disclosed chemical matter reported to target kinases, as well as access to more than six thousand protein kinase X-ray structures in the Protein Data Bank (PDB) (Roskoski, Pharmacol Res. 144:19-50 (2019)) to guide the positioning of linker exit vectors compatible with compound binding, we developed a large library of kinase-targeting degraders as a toolset to define the degradable kinome (Table 1). This library was designed to incorporate a wide range of kinase targeting scaffolds and binding modes, including Type I, Type II and allosteric. These parental molecules were derived from numerous sources including; FDA approved small molecules, such as imatinib (Gleevec®), and ibrutinib (Imbruvica®), where degraders that can overcome clinical resistance may be of value, patents, publications and novel in-house kinase targeting ligands (FIG. 1C: Table 1). Finally, several degraders were synthesized based on highly multi-kinase targeted inhibitors, such as desmethoxy-TAE684, AT7519 and ponatinib. Based on reported and in-house biochemical data, the parental inhibitors corresponding to degraders profiled described herein are able to engage 370 of the 395 unique kinases present in the DiscovRX kinomeSCAN® panel (93%), corresponding to at least 70% coverage of the human kinome, enabling large scale investigation of the relative degradability of kinases (FIG. 1D, FIG. 1E; FIG. 8A-FIG. 8B). To increase the probability of favorable ternary complex formation between target kinase and recruiting ligase, a variety of linker lengths, compositions, and attachment chemistries were employed, and E3-recruiting ligands targeting both CRBN and VHL were incorporated into the library design (FIG. 1C; Table 1).

TABLE 1
Library compounds and degraded kinases.

| | | | Total no. |
|---|---|---|---|
| Compound | | | kinase |
| name | Structure | Kinase hits | hits |
| BJG-04-201 | 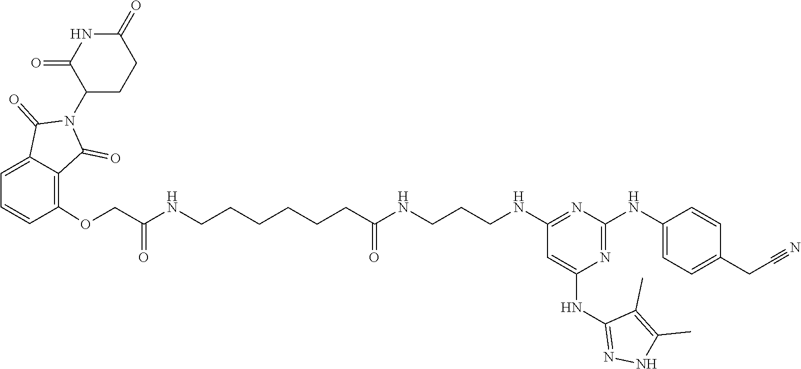 [SMILES: Cc1[nH]nc(Nc2cc(NCCCNC(=O)CCCCCCNC(=O)COc3cccc4c3C(=O)N(C3CCC(=O)NC3=O)C4=O)nc(Nc3ccc(CC#N)cc3)n2)c1C] | NA | NA |
| BJG-04-202 | 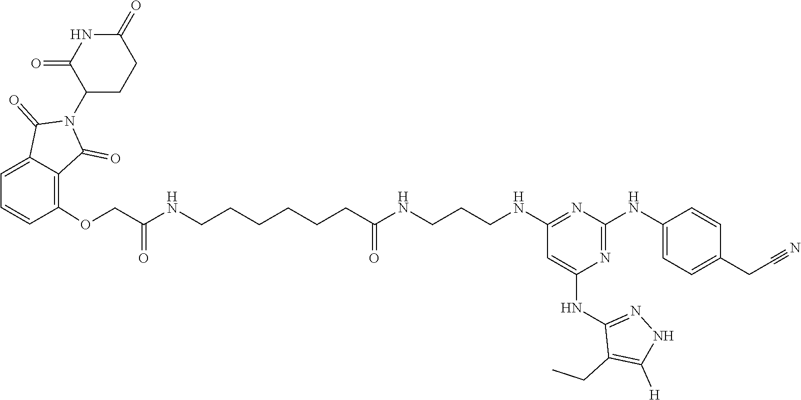 [SMILES: CCc1c[nH]nc1Nc1cc(NCCCNC(=O)CCCCCCNC(=O)COc2cccc3c2C(=O)N(C2CCC(=O)NC2=O)C3=O)nc(Nc2ccc(CC#N)cc2)n1] | NA | NA |
| BJG-04-203 | 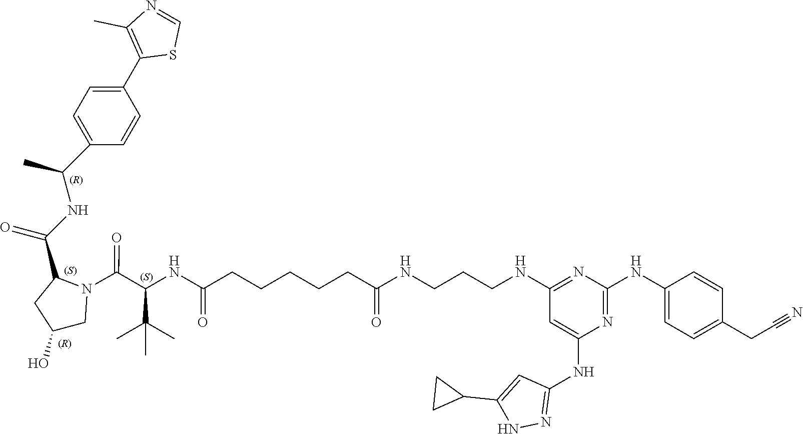 | AAK1, AURKA, AURKB, BMP2K, CDK9, EPHB2, GSK3B, ITK, LATS1, MAP4K2, NEK9, PAK4, PLKA, STK17B | 14 |
| BJG-05-020 | 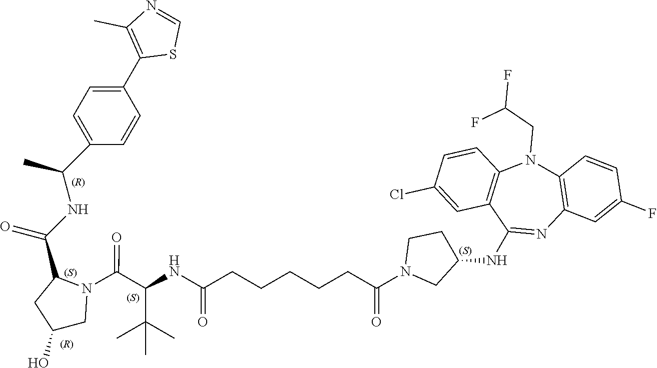 | NA | NA |
| BSJ-01-096 | 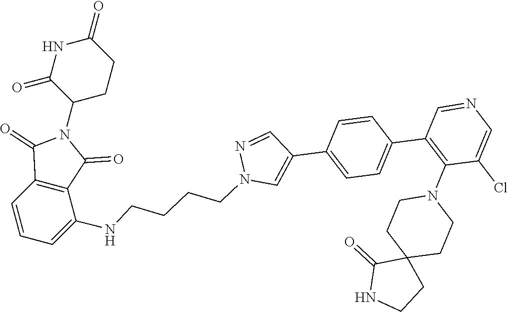 [SMILES: O=C1CCC(N2C(=O)c3cccc(NCCCCn4cc(-c5ccc(-c6cncc(Cl)c6N6CCC7(CCNC7=O)CC6)cc5)cn4)c3C2=O)C(=O)N1] | NA | NA |
| BSJ-01-147 | 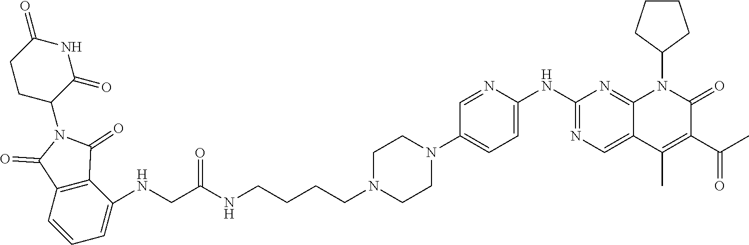 [SMILES: CC(=O)c1c(C)c2cnc(Nc3ccc(N4CCN(CCCCNC(=O)CNc5cccc6c5C(=O)N(C5CCC(=O)NC5=O)C6=O)CC4)cn3)nc2n(C2CCCC2)c1=O] | CDK4, CDK6, WEE1 | 3 |
| BSJ-03-123 | 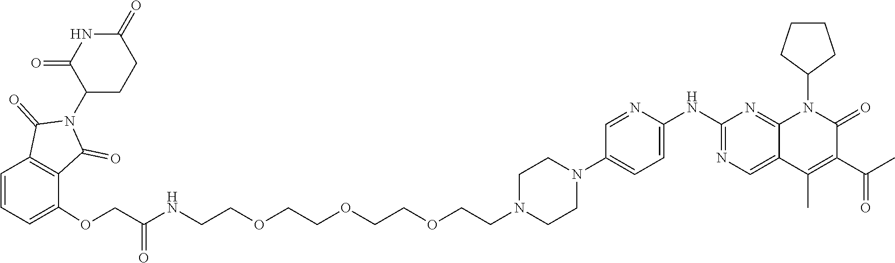 [SMILES: CC(=O)c1c(C)c2cnc(Nc3ccc(N4CCN(CCOCCOCCOCCNC(=O)COc5cccc6c5C(=O)N(C5CCC(=O)NC5=O)C6=O)CC4)cn3)nc2n(C2CCCC2)c1=O] | BCKDK, CDK4, CDK6, COQ8A, PDK1, PDK2, PDK3, WEE1 | 8 |
| BSJ-03-204 | 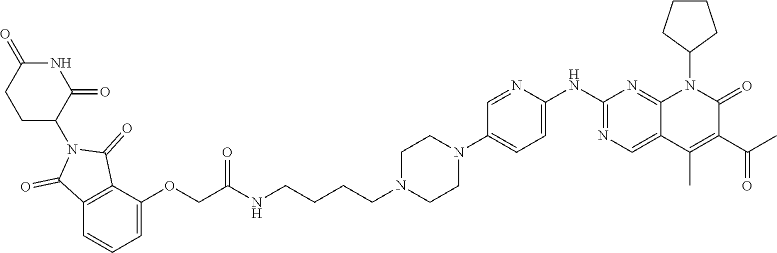 [SMILES: CC(=O)c1c(C)c2cnc(Nc3ccc(N4CCN(CCCCNC(=O)COc5cccc6c5C(=O)N(C5CCC(=O)NC5=O)C6=O)CC4)cn3)nc2n(C2CCCC2)c1=O] | CDK4, CDK6, PDK1, WEE1 | 4 |
| BSJ-04-132 | 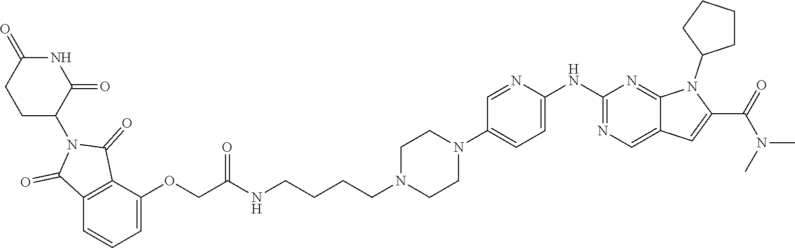 [SMILES: CN(C)C(=O)c1cc2cnc(Nc3ccc(N4CCN(CCCCNC(=O)COc5cccc6c5C(=O)N(C5CCC(=O)NC5=O)C6=O)CC4)cn3)nc2n1C1CCCC1] | BCKDK, CDK4, CDK6, COQ8A, PDK1, PDK2, PDK3, | 3 |
| BSJ-04-178 | 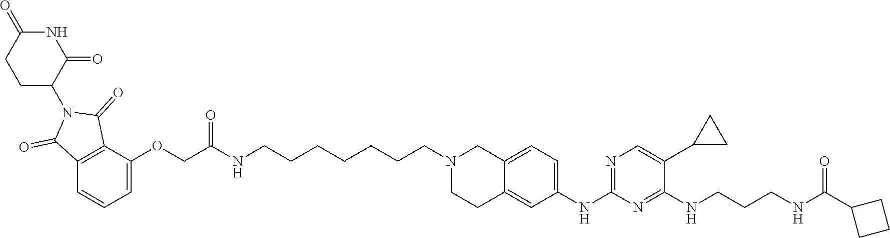 [SMILES: O=C(COc1cccc2c1C(=O)N(C1CCC(=O)NC1=O)C2=O)NCCCCCCCN1CCc2cc(Nc3ncc(C4CC4)c(NCCCNC(=O)C4CCC4)n3)ccc2C1] | AAK1, AURKA, AURKB, BMP2K, CDK10, CDK9, GAK, MARK2, MARK3, MARK4, SIK2, STKI7A, STK17B, SNRK, ULK1, WEE1 | 16 |
| BSJ-05-026 | 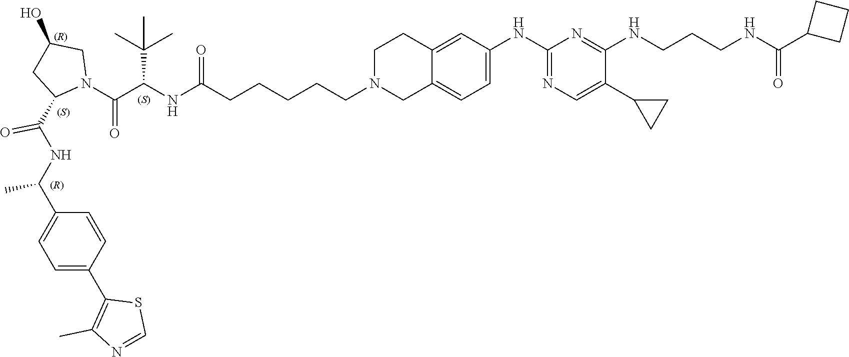 | AAK1, GAK, MARK2, MARK3, MARK4, RPS6KB1, SIK2, SIK3, SNRK, STKI7A, STKI7B, ULK1, WEE1 | 13 |
| dAbl-2 | 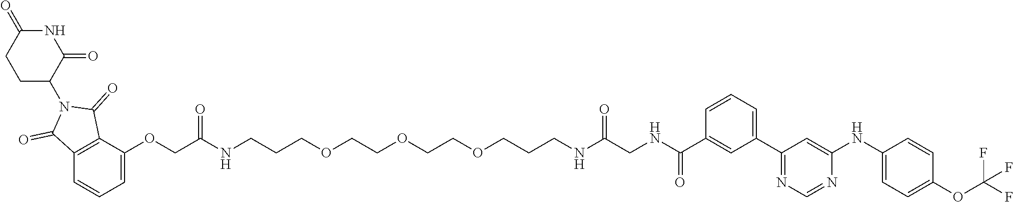 [SMILES: O=C(CNC(=O)c1cccc(-c2cc(Nc3ccc(OC(F)(F)F)cc3)ncn2)c1)NCCCOCCOCCOCCCNC(=O)COc1cccc2c1C(=O)N(C1CCC(=O)NC1=O)C2=O] | NA | NA |
| dAURK-4 | 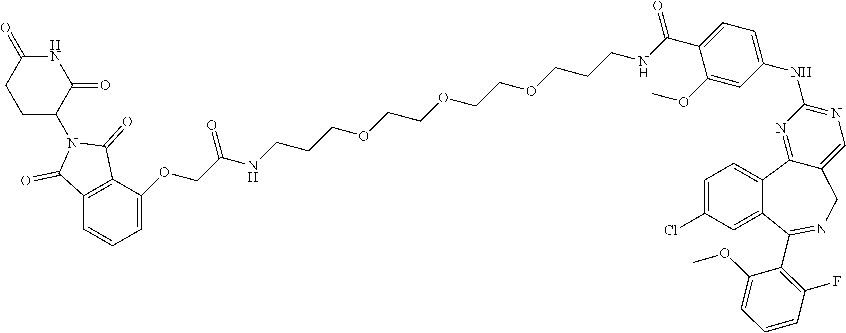 [SMILES: COc1cc(Nc2ncc3c(n2)-c2ccc(Cl)cc2C(c2c(F)cccc2OC)=NC3)ccc1C(=O)NCCCOCCOCCOCCCNC(=O)COc1cccc2c1C(=O)N(C1CCC(=O)NC1=O)C2=O] | AURKA, AURKB | 2 |
| DB0614 | 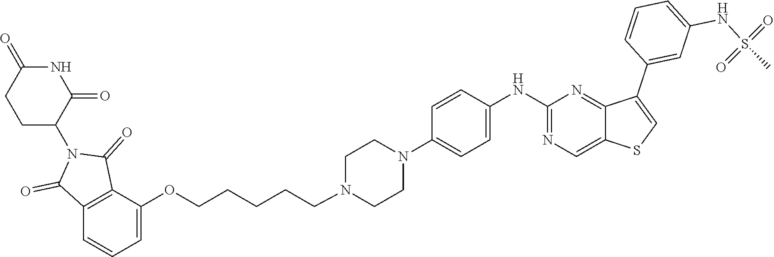 | AAK1, AURKA, BMP2K, CAMKK1, CDK16, CDK4, CDK6, EIF2AK2, FER, GAK, LCK, LIMK2, MAP3K11, MAPK8, MAPK9, NEK9, PLK4, PTK2B, SIK2, STKI7A, STKI7B, ULK1, ULK3, WEE1 | 24 |
| DB0646 | 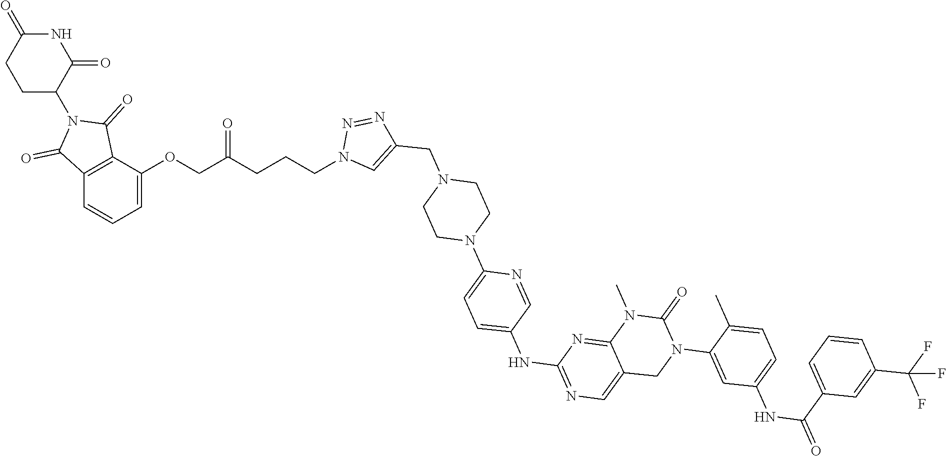 [SMILES: Cc1ccc(NC(=O)c2cccc(C(F)(F)F)c2)cc1N1Cc2cnc(Nc3ccc(N4CCN(Cc5cn(CCCC(=O)COc6cccc7c6C(=O)N(C6CCC(=O)NC6=O)C7=O)nn5)CC4)nc3)nc2N(C)C1=O] | ABL2, AURKA, BLK, BUB1, CDK11A, CDK11B, CDK4, CSK, DDR2, EPHA3, EPHB3, EPHB6, FER, FYN, GAK, LATS1, LCK, LIMK1, LIMK2, LRRK2, LYN, MAP3K1, MAP3K11, MAP3K20, MAP4K1, MAP4K2, MAP4K5, MAPK11, MAPK12, MAPK14, MAPK8, MAPK9, MAPKAPK2, MKNK2, NLK, PLK1, PTK2, PTK2B, RIPK1, RIPK2, RPS6KA3, SIK2, SRC, TAOK2, TEC, TNK2, TTK, UHMK1, ULK1, WEE1, YES1 | 33 |
| DB0661 | 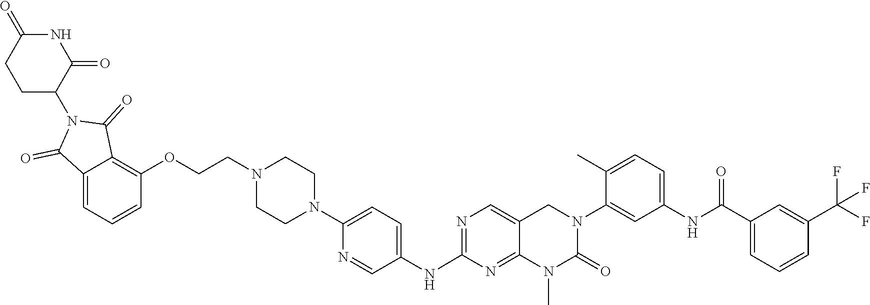 [SMILES: Cc1ccc(NC(=O)c2cccc(C(F)(F)F)c2)cc1N1Cc2cnc(Nc3ccc(N4CCN(CCOc5cccc6c5C(=O)N(C5CCC(=O)NC5=O)C6=O)CC4)nc3)nc2N(C)C1=O] | BLK, CDK4, CLK1, CSK, FER, LCK, LIMK1, MAPK8, MAPK9, MKNK2, PLK1, PTK2B, SIKa2, SRC, TNK2, UHMK1, YES1 | 17 |
| DB0662 | 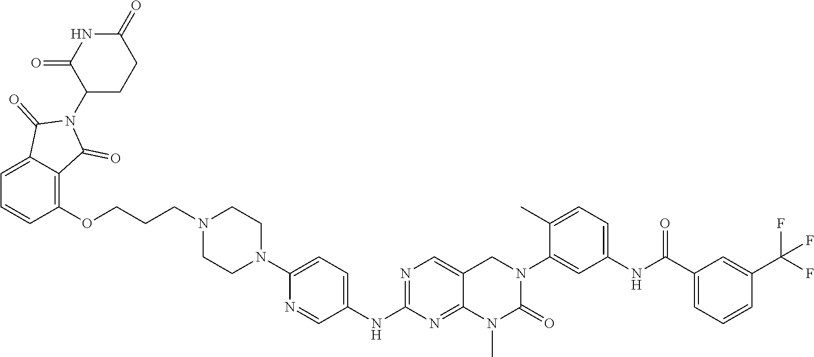 [SMILES: Cc1ccc(NC(=O)c2cccc(C(F)(F)F)c2)cc1N1Cc2cnc(Nc3ccc(N4CCN(CCCOc5cccc6c5C(=O)N(C5CCC(=O)NC5=O)C6=O)CC4)nc3)nc2N(C)C1=O] | ABL1, ABL2, BLK, BUB1, CDK11B, CDK4, CSK, EPHB6, FER, FYN, GAK, LCK, LIMK1, MAP3K1, MAP3K11, MAP3K20, MAP4K1, MAPK14, MAPK8, MAPK9, MAPKAPK2, MKNK2, PAK4, PDIK1L, PTK2B, RPS6KA1, RPS6KA3, SIK2, SRC, TNK2, UHMK1, ULK1,YES1 | 33 |
| DB0663 | 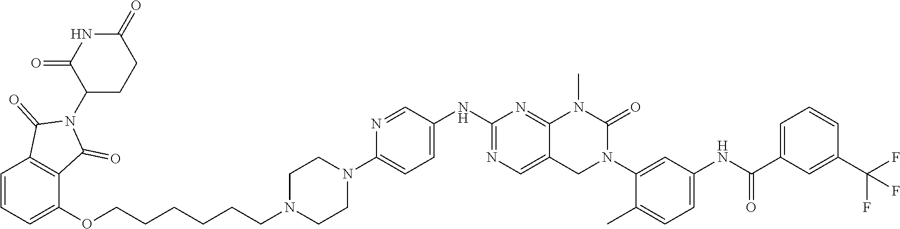 [SMILES: Cc1ccc(NC(=O)c2cccc(C(F)(F)F)c2)cc1N1Cc2cnc(Nc3ccc(N4CCN(CCCCCCOc5cccc6c5C(=O)N(C5CCC(=O)NC5=O)C6=O)CC4)nc3)nc2N(C)C1=O] | CDK4, BLK, FER, LIMK2, GAK, CSK, SIK2, LCK, PTK2B, SRC, ABL2, MAPK14.a MAPK9, MAP4K2, MKNK2, MAP3K20, TNK2 | 17 |
| DB1113 | 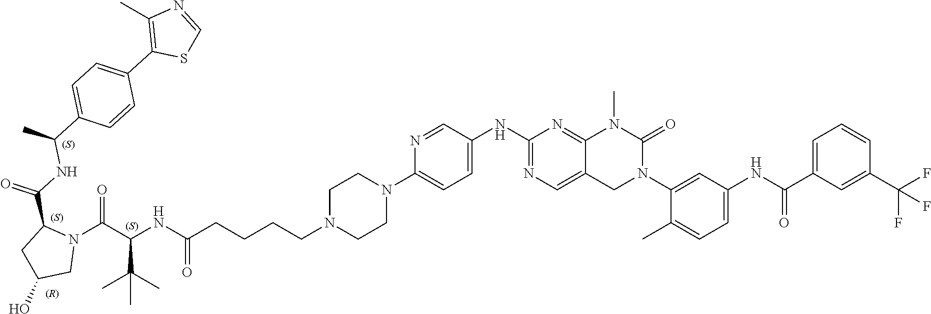 | ABL1, ABL2, BLK, CDK11B, CDK4, CSK, EPHA3, FER, GAK, LIMK1, MAP3K20, MAP4K1, MAP4K2, MAP4K3, MAP4K5, MAPK14, MAPK7, MAPK8, MAPK9, MAPKAPK2, MAPKAPK3, NLK, PDIK1L, PTK2B, RIPK1, RPS6KA1, RPS6KA3, SIK2, SIK3, STK35, TNK2, ULK1 | 31 |
| DB1114 | 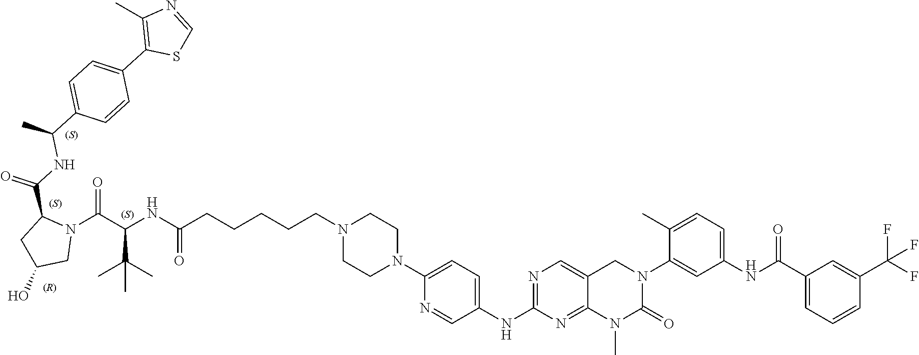 | ABL1, ABL2, BLK, CDK11B, CDK4, CIT, CSK, EPHA3, FER, GAK, LCK, LIMK2, MAP3K20, MAP3K7, MAP4K1, MAP4K2, MAP4K5, MAPK14, MAPK7, MAPK9, MAPKAPK2, MAPKAPK3, PDIK1L, PTK2B, RIPK1, RPS6KA1, SIK2, STK35, TAOK2, TNK2, ULK1 | 31 |
| DB117034 | 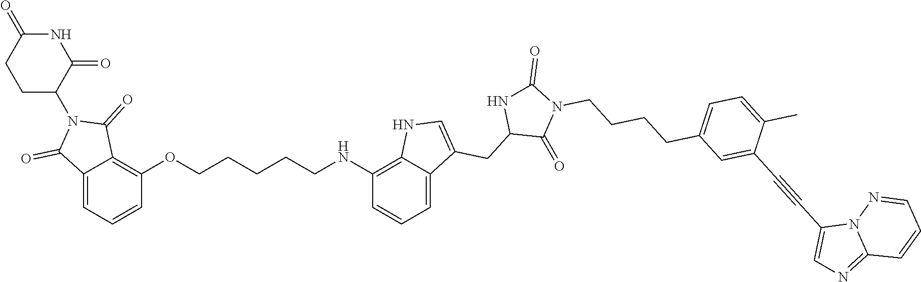 [SMILES: Cc1ccc(CCCCN2C(=O)NC(Cc3c[nH]c4c(NCCCCCOc5cccc6c5C(=O)N(C5CCC(=O)NC5=O)C6=O)cccc34)C2=O)cc1C#Cc1cnc2cccnn12] | NA | NA |
| DB118430 | 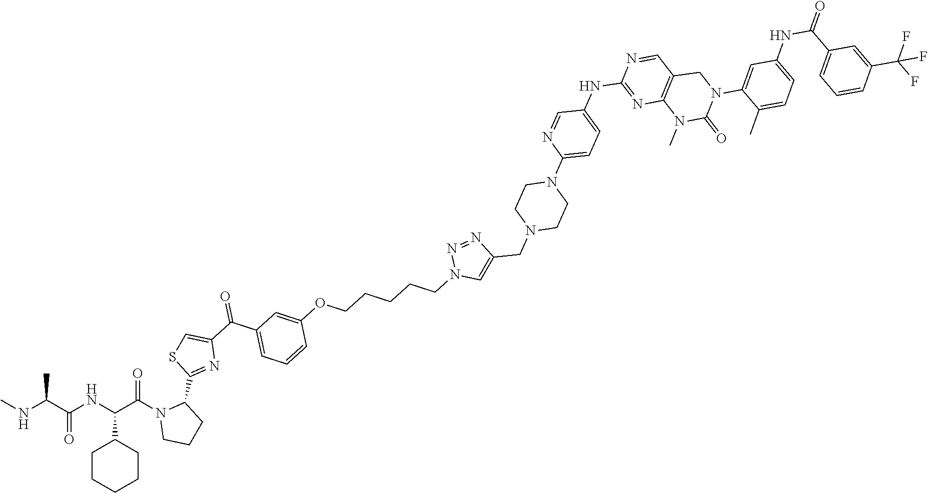 [SMILES: CN[C@@H](C)C(=O)N[C@H](C(=O)N1CCC[C@H]1c1nc(C(=O)c2cccc(OCCCCCn3cc(CN4CCN(c5ccc(Nc6ncc7c(n6)N(C)C(=O)N(c6cc(NC(=O)c8cccc(C(F)(F)F)c8)ccc6C)C7)cn5)CC4)nn3)c2)cs1)C1CCCCC1] | BLK, BUB1, CDK4, LIMK2, SIK2, STK17A, TEC, TNK2, UHMK1 | 9 |
| DB-3-291 | 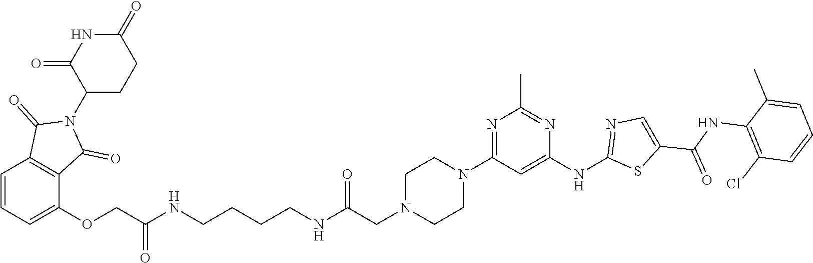 [SMILES: Cc1nc(Nc2ncc(C(=O)Nc3c(C)cccc3Cl)s2)cc(N2CCN(CC(=O)NCCCCNC(=O)COc3cccc4c3C(=O)N(C3CCC(=O)NC3=O)C4=O)CC2)n1] | ABL2, BLK, CSK, WEE1 | 4 |
| DD-02-198 | 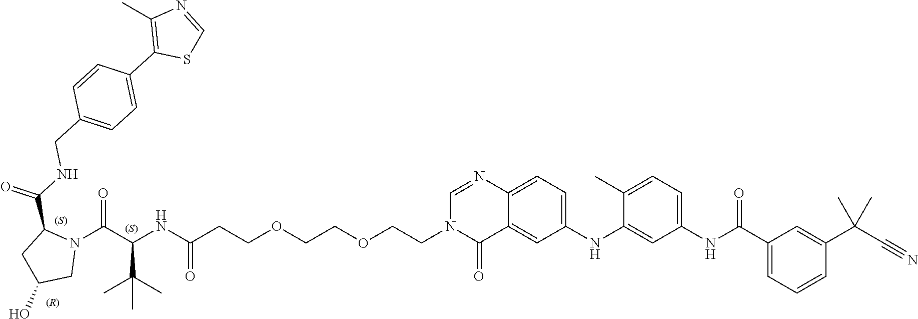 | MAPK14 | 1 |
| DD-03-106 | 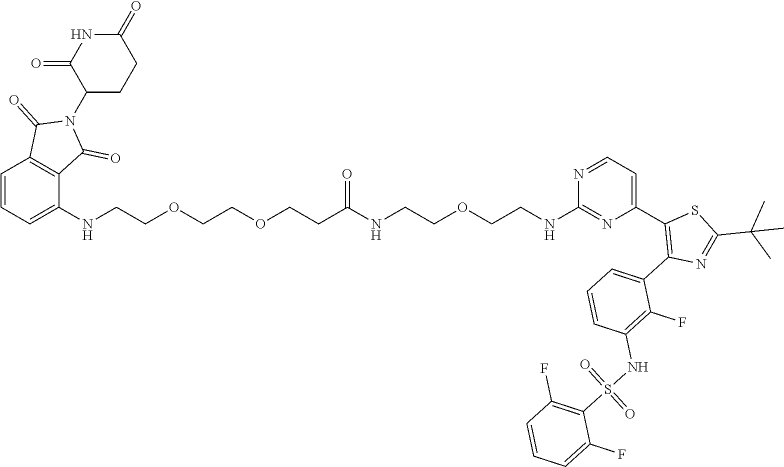 [SMILES: CC(C)(C)c1nc(-c2cccc(NS(=O)(=O)c3c(F)cccc3F)c2F)c(-c2ccnc(NCCOCCNC(=O)CCOCCOCCNc3cccc4c3C(=O)N(C3CCC(=O)NC3=O)C4=O)n2)s1] | AURKA, BCKDK, CDK1, CDK16, CDK17, CDK2, CDK3, CDK4, CDK6, COQ8A, COQ8B, CSK, EIF2AK2, LIMK1, LIMK2, MAP3K20, NLK, PLK1, PDK1, PDK2, TESK2 | 21 |
| DD-03-107 | 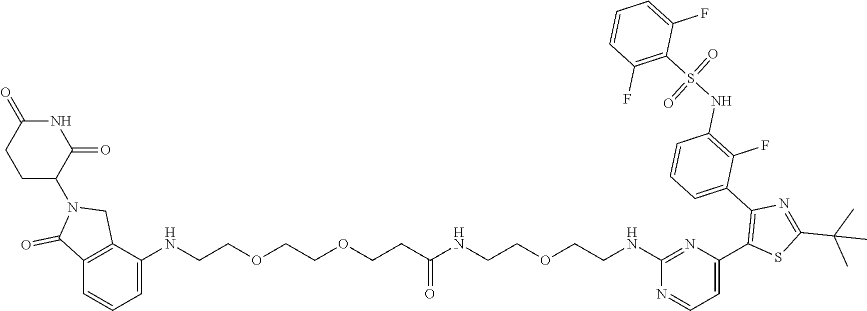 [SMILES: CC(C)(C)c1nc(-c2cccc(NS(=O)(=O)c3c(F)cccc3F)c2F)c(-c2ccnc(NCCOCCNC(=O)CCOCCOCCNc3cccc4c3CN(C3CCC(=O)NC3=O)C4=O)n2)s1] | BCKDK, COQ8A, LIMK1, PDK1, PDK2, PDK3 | 6 |
| DD-03-119 | 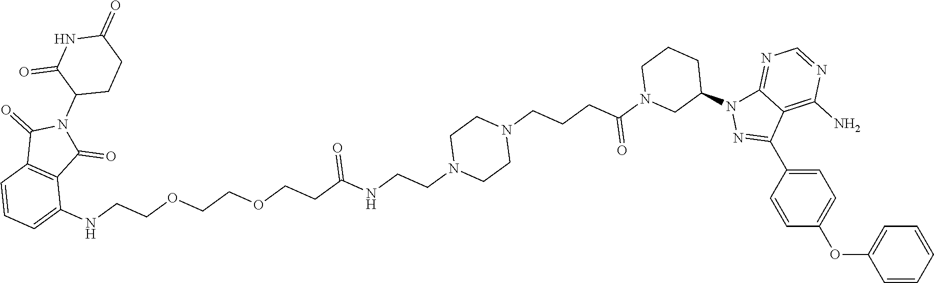 [SMILES: Nc1ncnc2c1c(-c1ccc(Oc3ccccc3)cc1)nn2[C@@H]1CCCN(C(=O)CCCN2CCN(CCNC(=O)CCOCCOCCNc3cccc4c3C(=O)N(C3CCC(=O)NC3=O)C4=O)CC2)C1] | ABL2, BLK, CSK, FYN, LCK, SRC, TEC | 3 |
| DD-03-156 | 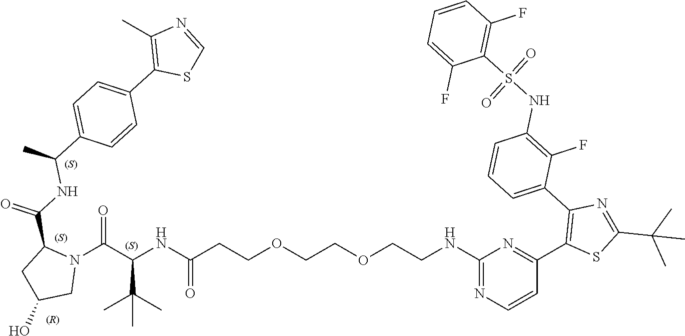 | CDK17, LIMK1, LIMK2 | 3 |
| DD-03-171 | 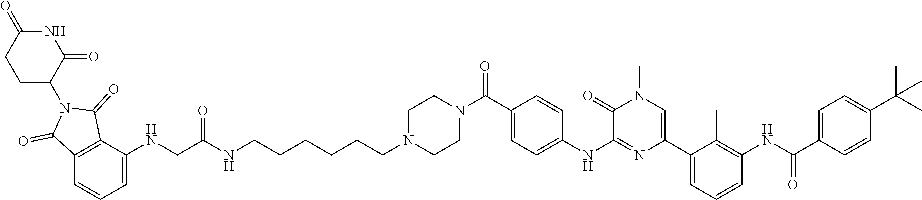 [SMILES: Cc1c(NC(=O)c2ccc(C(C)(C)C)cc2)cccc1-c1cn(C)c(=O)c(Nc2ccc(C(=O)N3CCN(CCCCCCNC(=O)CNc4cccc5c4C(=O)N(C4CCC(=O)NC4=O)C5=O)CC3)cc2)n1] | BTK | 1 |
| DD-04-118 | 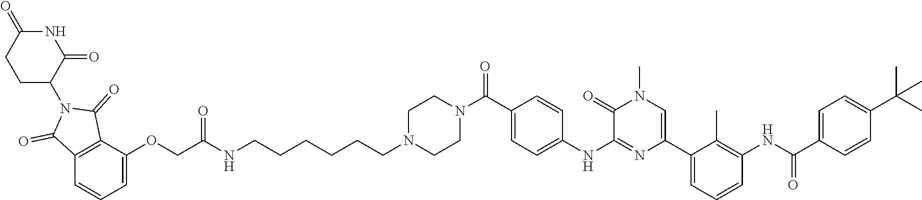 [SMILES: Cc1c(NC(=O)c2ccc(C(C)(C)C)cc2)cccc1-c1cn(C)c(=O)c(Nc2ccc(C(=O)N3CCN(CCCCCCNC(=O)COc4cccc5c4C(=O)N(C4CCC(=O)NC4=O)C5=O)CC3)cc2)n1] | BTK | 1 |
| dFLT-2 | 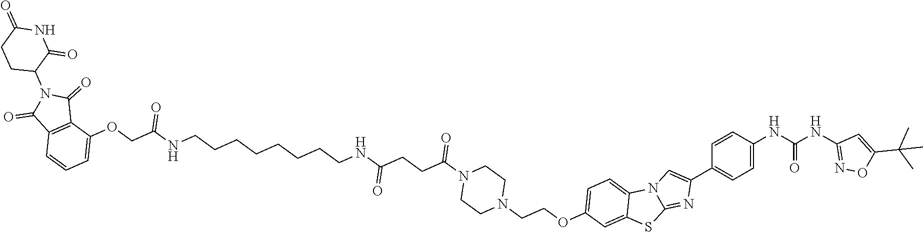 [SMILES: CC(C)(C)c1cc(NC(=O)Nc2ccc(-c3cn4c(n3)sc3cc(OCCN5CCN(C(=O)CCC(=O)NCCCCCCCCNC(=O)COc6cccc7c6C(=O)N(C6CCC(=O)NC6=O)C7=O)CC5)ccc34)cc2)no1] | NA | NA |
| dKIT-2 | 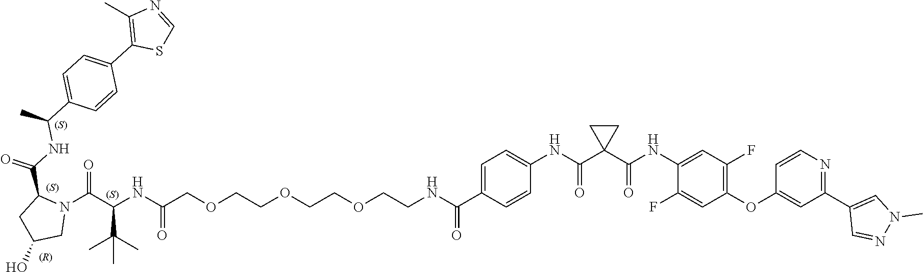 | NA | NA |
| dRAF-3 | 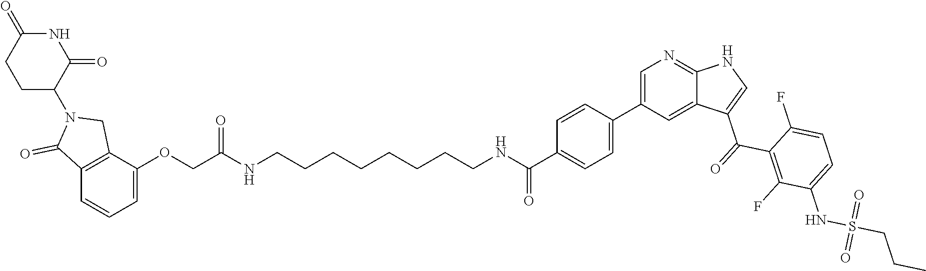 [SMILES: CCCS(=O)(=O)Nc1ccc(F)c(C(=O)c2c[nH]c3ncc(-c4ccc(C(=O)NCCCCCCCCNC(=O)COc5cccc6c5CN(C5CCC(=O)NC5=O)C6=O)cc4)cc23)c1F] | BLK, CSK, LCK, LIMK2, MAP2K5, MAP3K20 | 6 |
| FMF-02- 153-1 | 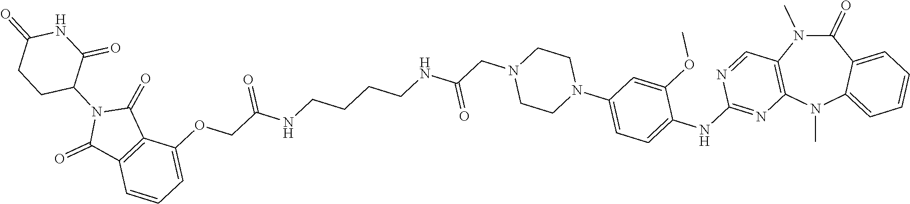 [SMILES: COc1cc(N2CCN(CC(=O)NCCCCNC(=O)COc3cccc4c3C(=O)N(C3CCC(=O)NC3=O)C4=O)CC2)ccc1Nc1ncc2c(n1)N(C)c1ccccc1C(=O)N2C] | NA | NA |
| FMF-04- 135-1 | 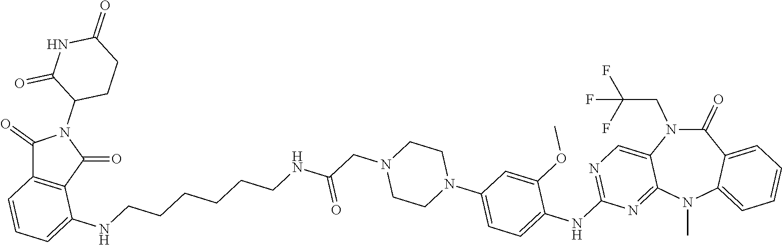 [SMILES: COc1cc(N2CCN(CC(=O)NCCCCCCNc3cccc4c3C(=O)N(C3CCC(=O)NC3=O)C4=O)CC2)ccc1Nc1ncc2c(n1)N(C)c1ccccc1C(=O)N2CC(F)(F)F] | NA | NA |
| FMF-04- 147-1 | 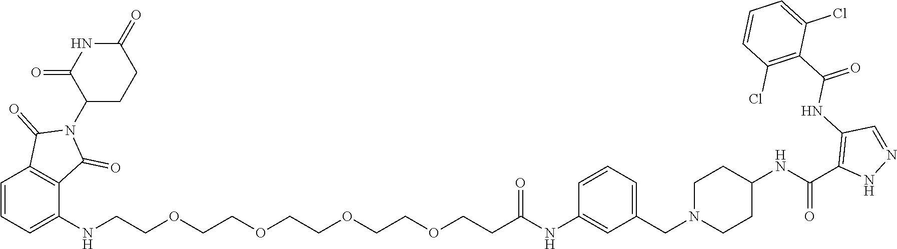 [SMILES: O=C1CCC(N2C(=O)c3cccc(NCCOCCOCCOCCOCCC(=O)Nc4cccc(CN5CCC(NC(=O)c6[nH]ncc6NC(=O)c6c(Cl)cccc6Cl)CC5)c4)c3C2=O)C(=O)N1] | CDK11A, CDK9, CLK1, GSK3A, GSK3B, PIK3CG, SGK3 | 7 |
| FMF-03- 178-1 | 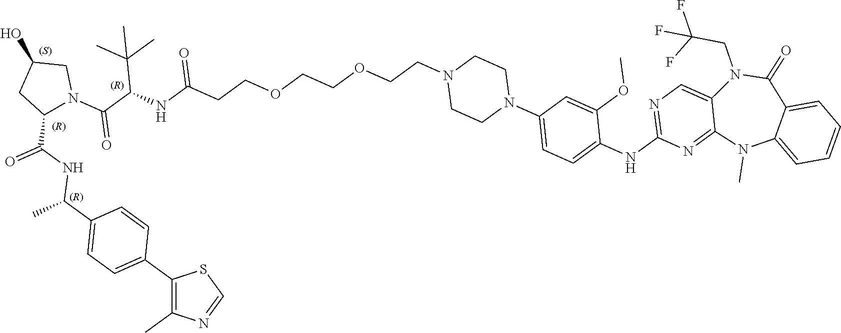 | BLK, CSK | 2 |
| FMF-06- 098-1 | 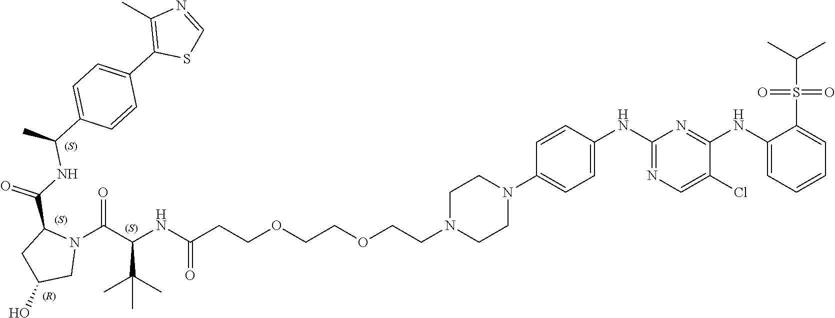 | AAK1, ABL2, AURKA, AURKB, BUB1B, CDC7, CDK1, CDK12, CDK13, CDK2, CDK4, CDk6, CDK7, CDK9, CHEK1, CSNK1D, EPHA1, FER, FGFR1, GAK, IRAK4, ITK, LIMK2, MAP4K2, MAP4K3, MAPK6, MAPK7, MARK4, MELK, PKN3, PLK4, PRKAA1, PTK2, PTK6, RPS6KA4, SIK2, STK35, TNK2, UHMK1, ULK1, WEE1 | 42 |
| FMF-06- 104-1 | 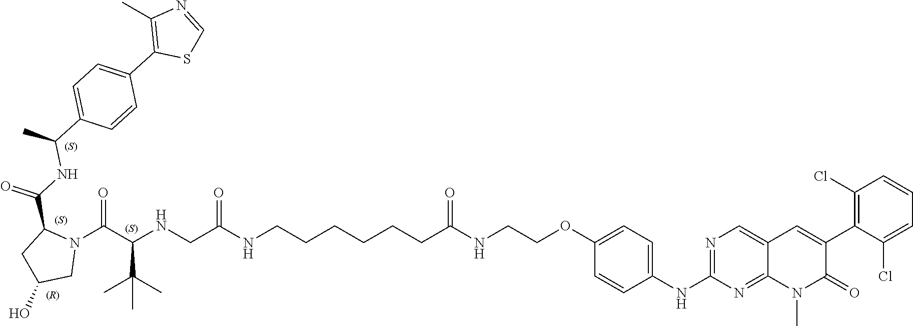 | NA | NA |
| ImatinIMiD- 2 | 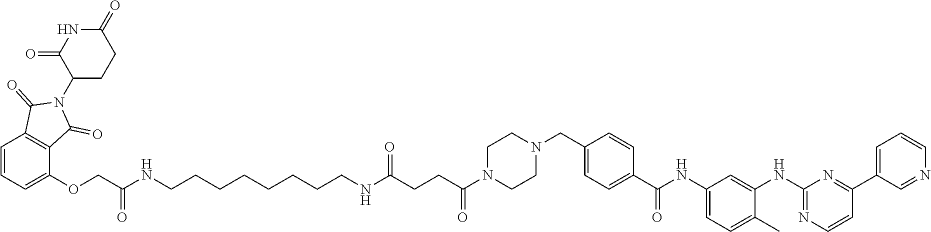 [SMILES: Cc1ccc(NC(=O)c2ccc(CN3CCN(C(=O)CCC(=O)NCCCCCCCCNC(=O)COc4cccc5c4C(=O)N(C4CCC(=O)NC4=O)C5=O)CC3)cc2)cc1Nc1nccc(-c2cccnc2)n1] | NA | NA |
| ImatinIMiD- 5 | 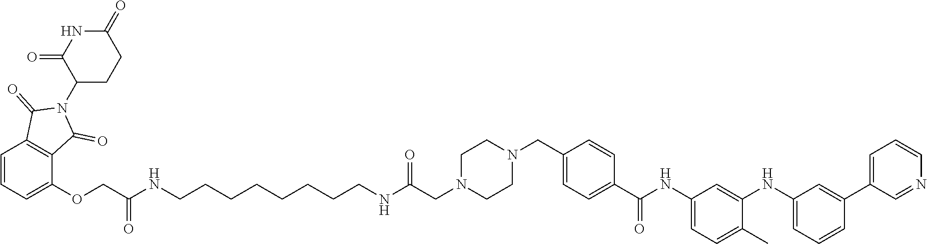 [SMILES: Cc1ccc(NC(=O)c2ccc(CN3CCN(CC(=O)NCCCCCCCCNC(=O)COc4cccc5c4C(=O)N(C4CCC(=O)NC4=O)C5=O)CC3)cc2)cc1Nc1cccc(-c2cccnc2)c1] | NA | NA |
| INY-01- 140-1 | 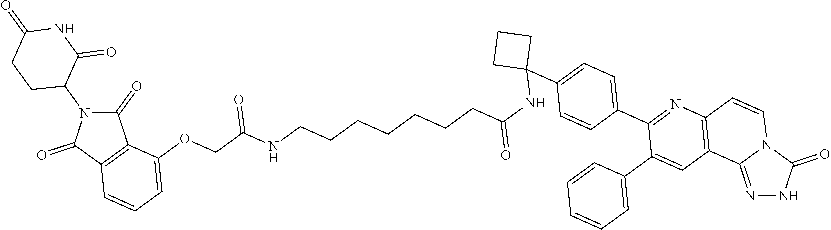 [SMILES: O=C(COc1cccc2c1C(=O)N(C1CCC(=O)NC1=O)C2=O)NCCCCCCCC(=O)NC1(c2ccc(-c3nc4ccn5c(=O)[nH]nc5c4cc3-c3ccccc3)cc2)CCC1] | PDK1, PDK2, PDK3 | 3 |
| INY-02- 083-01 | 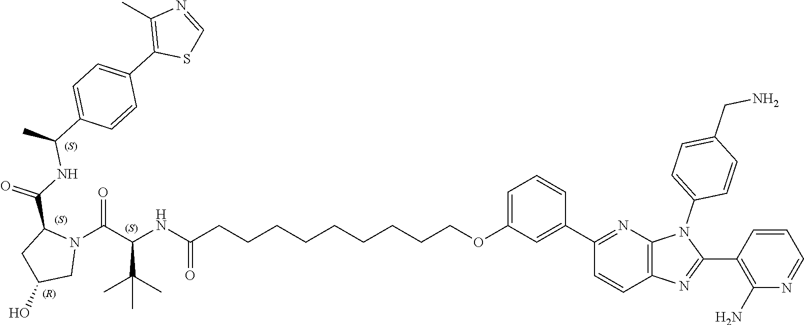 | NA | NA |
| INY-02-105 | 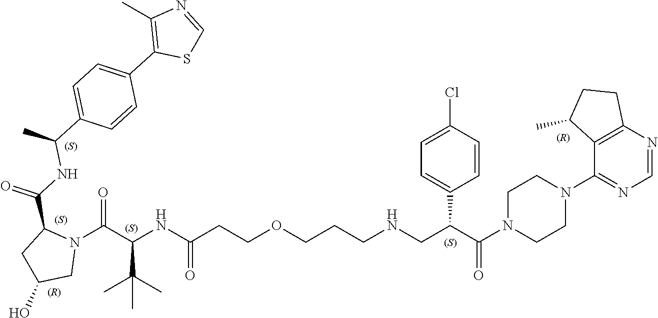 | NA | NA |
| INY-02-164 | 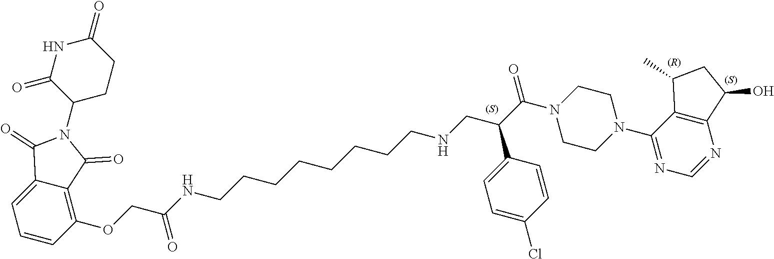 | AKT1, AKT2, AKT3, BCKDK, PDK1 | 5 |
| INY-02- 180-01 | 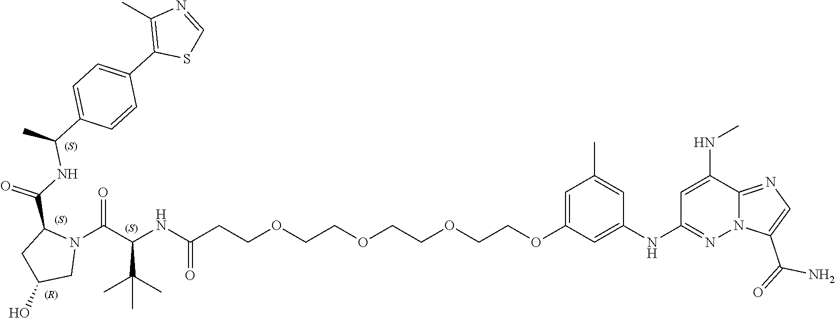 | NA | NA |
| INY-03- 041-01 | 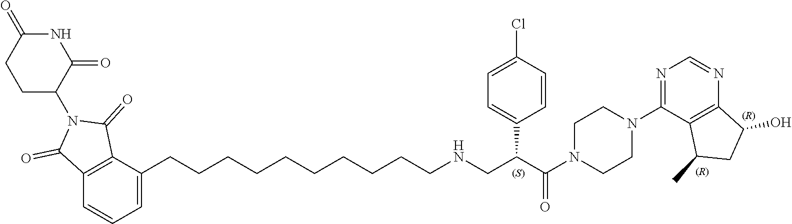 | AKT1, AKT2, AKT3, CDK4, MAPK6, STK17A | 6 |
| INY-04- 026-01 | 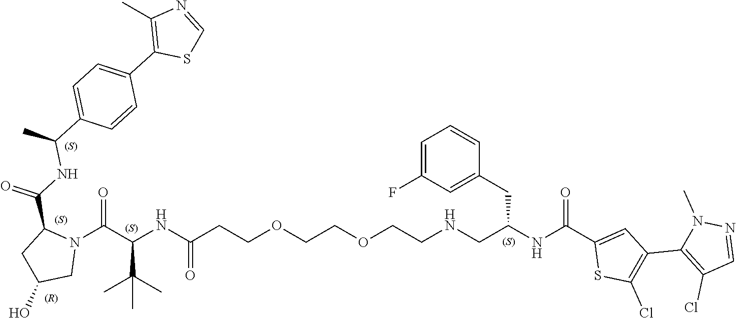 | NA | NA |
| JH-XI-10-02 | 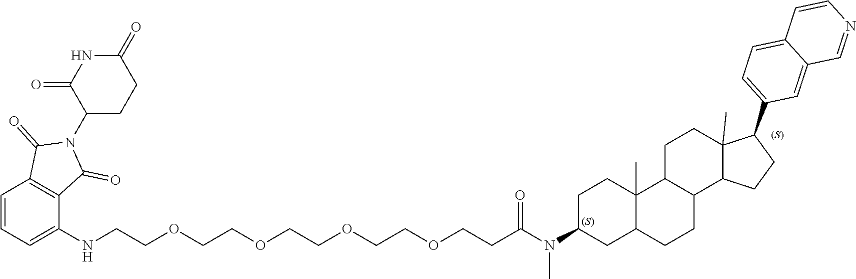 | NA | NA |
| INY-04- 125-01 | 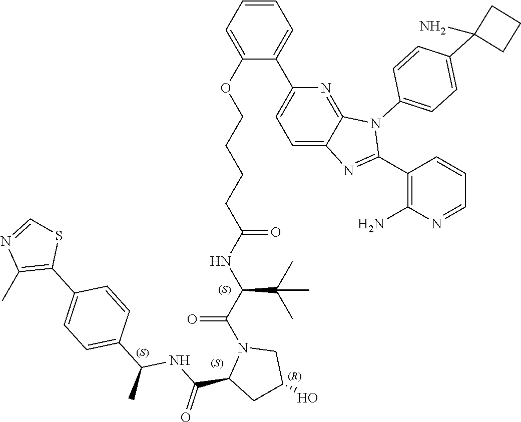 | LATS1, STK17A | 2 |
| JWG-118 | 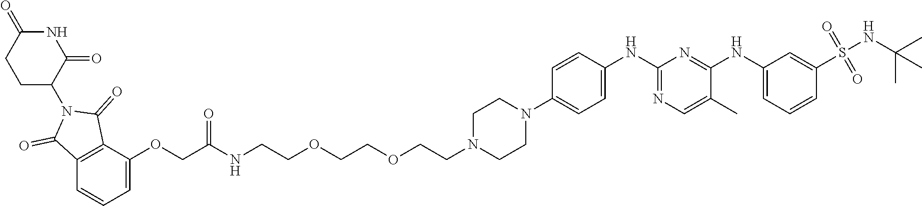 [SMILES: Cc1cnc(Nc2ccc(N3CCN(CCOCCOCCNC(=O)COc4cccc5c4C(=O)N(C4CCC(=O)NC4=O)C5=O)CC3)cc2)nc1Nc1cccc(S(=O)(=O)NC(C)(C)C)c1] | AAK1, AURKA, BMP2K, GAK, WEE1 | 5 |
| JWG-120 | 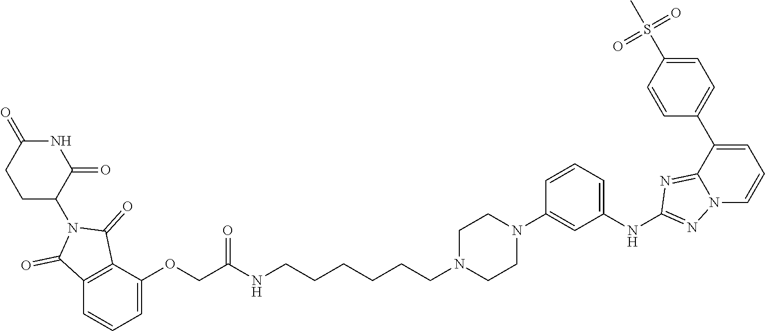 [SMILES: CS(=O)(=O)c1ccc(-c2cccn3nc(Nc4cccc(N5CCN(CCCCCCNC(=O)COc6cccc7c6C(=O)N(C6CCC(=O)NC6=O)C7=O)CC5)c4)nc23)cc1] | AAK1, AURKA | 2 |
| JWG-122 | 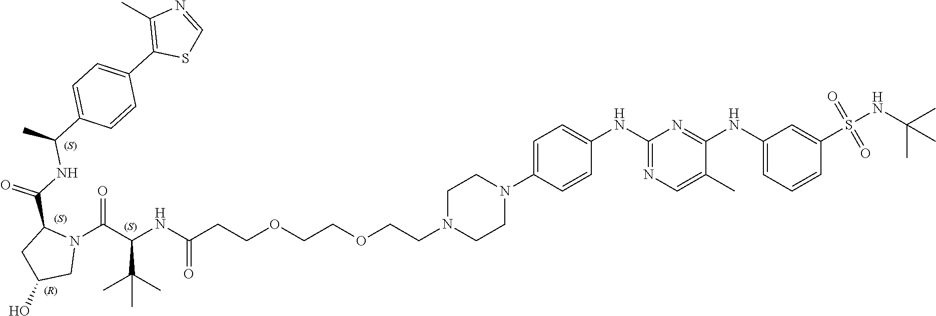 | AAK1, GAK | 2 |
| JWG-123 | 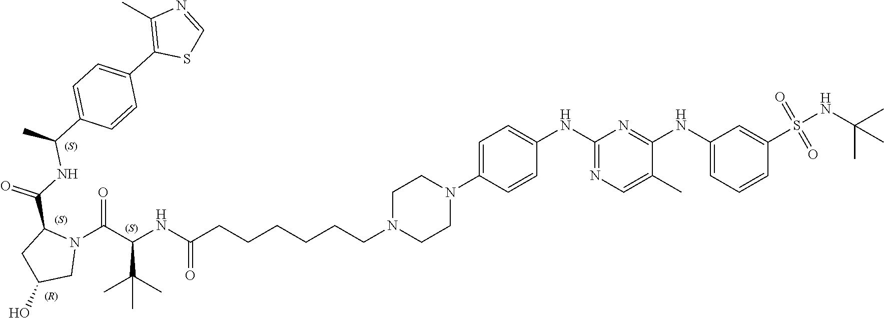 | AAK1, CDK16, WEE1, GAK, MARK4, NEK9, RPS6KB1, SIK2, SIK3, SNRK, STKI7A, STK17B | 12 |
| JWG-137 | 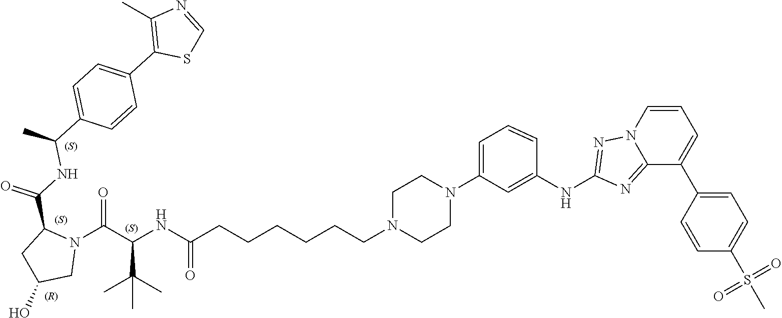 | NA | NA |
| JWG-148 | 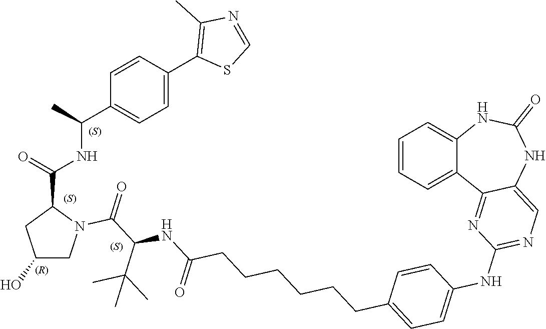 | ABL2, EPHB2, SIK2, TYK2 | 4 |
| LT2-49 | 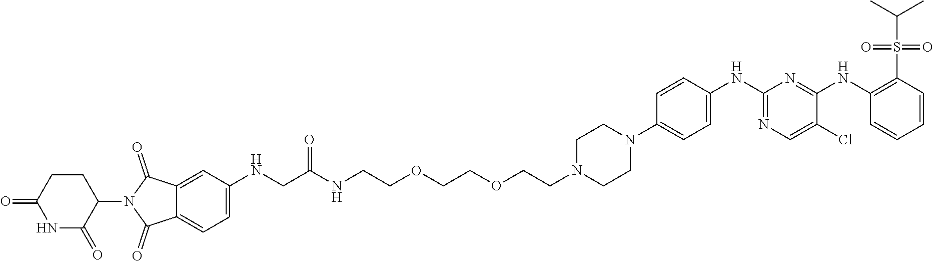 [SMILES: CC(C)S(=O)(=O)c1ccccc1Nc1nc(Nc2ccc(N3CCN(CCOCCOCCNC(=O)CNc4ccc5c(c4)C(=O)N(C4CCC(=O)NC4=O)C5=O)CC3)cc2)ncc1Cl] | CDK4, AURKA, WEE1, BLK, FER, CDK6, LIMK2, AAK1, CDK5, CDK2, ITK, CDK17, LCK, PTK2B, CDK9, CDK7, CDK13, PRKAA1, CDK12, BMP2K, STK10 | 21 |
| MFH-5-103- 1 | 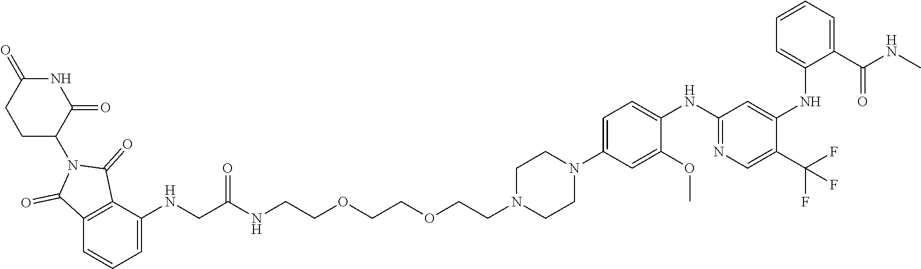 [SMILES: CNC(=O)c1ccccc1Nc1cc(Nc2ccc(N3CCN(CCOCCOCCNC(=O)CNc4cccc5c4C(=O)N(C4CCC(=O)NC4=O)C5=O)CC3)cc2OC)ncc1C(F)(F)F] | AURKA, CDK10, CDK7, MAPK7, PTK2B, RPS6KA1, RPS6KA3, STK33, WEE1 | 9 |
| MFH-5-115- 2 | 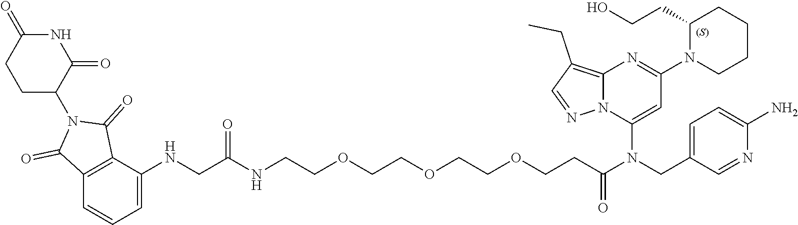 | CDK4, AURK4, WEE1, STK17A, PLK1, BUB1, TTK, UHMK1, MAP3K1, BUB1B, RIOK2, NUAK1, PIM2, CSNK1A1 | 14 |
| MFH-5-116- 1 | 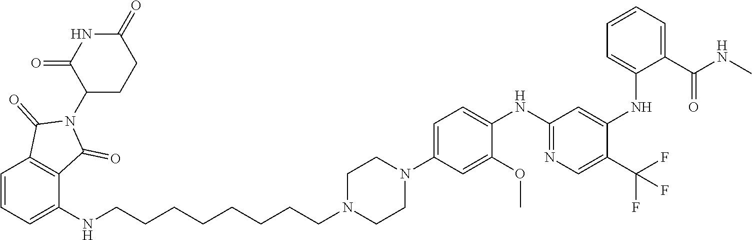 [SMILES: CNC(=O)c1ccccc1Nc1cc(Nc2ccc(N3CCN(CCCCCCCCNc4cccc5c4C(=O)N(C4CCC(=O)NC4=O)C5=O)CC3)cc2OC)ncc1C(F)(F)F] | AURKA, NUAK1, PTK2B, KPS6KA1, RPS6KA3, STK33, WEE1 | 7 |
| MFH-5-126- 1 | 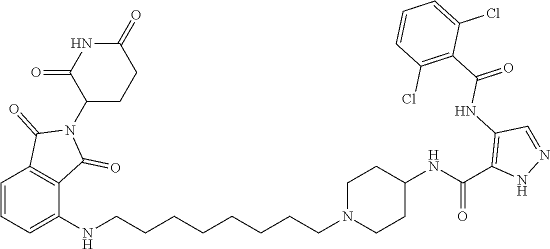 [SMILES: O=C1CCC(N2C(=O)c3cccc(NCCCCCCCCN4CCC(NC(=O)c5[nH]ncc5NC(=O)c5c(Cl)cccc5Cl)CC4)c3C2=O)C(=O)N1] | AURKA, BUB1, BUB1B, CDK13, CDK14, CDK17, CDK4, CDK9, CHEK1, CLK1, CSNK1A1, CSNK1D, DAPK1, ERN1, GSK3A, GSK3B, MAP3K1, NUAK1, PIK3CG, PIM2, PLK1, RIOK2, STK17A, STK17B, TTK, UHMK1, WEE1 | 27 |
| RSS0628 | 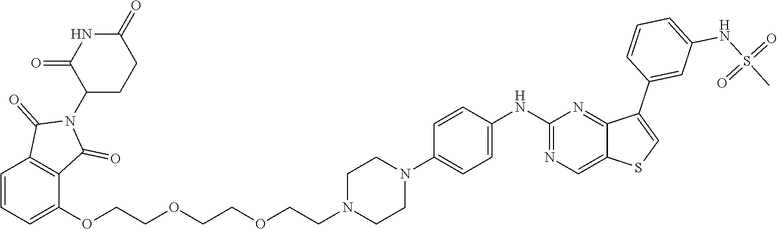 | AAK1, AURKA, CAMKK1, CDK4, CDK6, LIMK2, NEK9, PTK2B, STK17A, STK17B, ULK1, ULK3, WEE1 | 13 |
| RSS0680 | 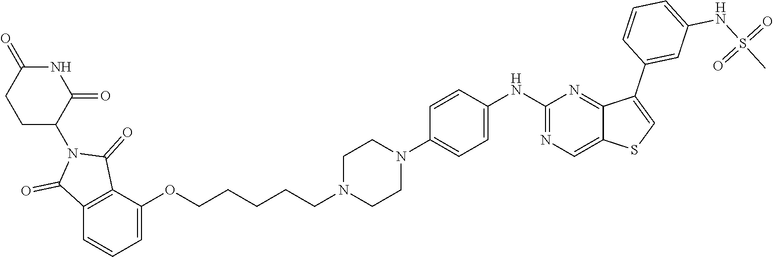 | AAK1, CDK1, CDK16, CDK2, CDKA, CDK6, EIF2AK4, GAK, LATS1, LIMK2, MAPK6, MAPKAPK5, MARK2, MARK4, MKNK2, NEK9, RPS6KB1, SIK2, SNRK, STK17A, STK17B. STK35, WEE1 | 26 |
| SB1-G-181 | 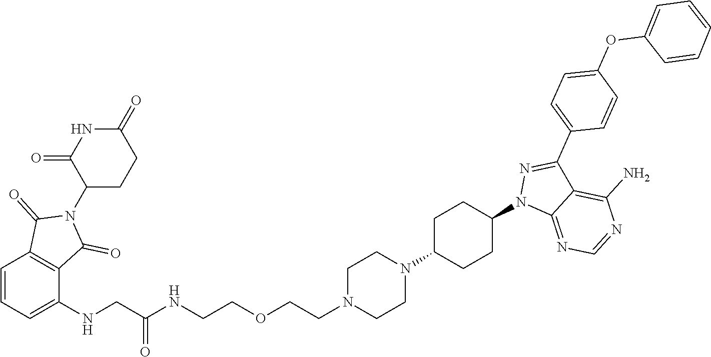 [SMILES: Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1] | ABL1, ABL2, AURKA, BLK, CSK, CSNK1E, FYN, LATS1, LCK, LIMK1, MAP2K5, SRC | 12 |
| SB1-G-187 | 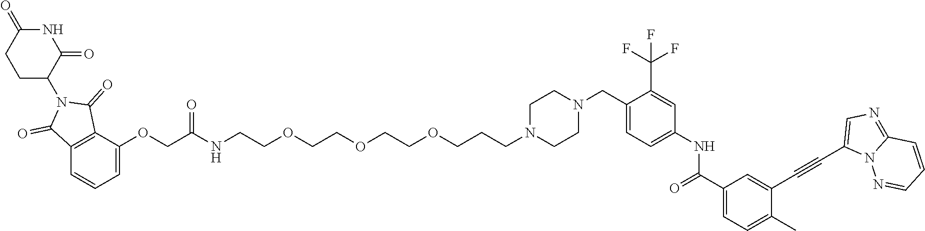 [SMILES: Cc1ccc(C(=O)Nc2ccc(CN3CCN(CCCOCCOCCOCCNC(=O)COc4cccc5c4C(=O)N(C4CCC(=O)NC4=O)C5=O)CC3)c(C(F)(F)F)c2)cc1C#Cc1cnc2cccnn12] | ABL1, ABL2, BLK, CDK14, CDK17, CDK5, CDK6, COQ8A, EPHA1, EPHA2, FER, FYN, GAK, IRAK1, LCK, LYN, MAP3K1, MAP3K20, MAP3K7, MAP4K2, MAP4K5, MAPK14, PDK1, PDK2, PDK3, RIPK1, RIPK2, SRC, STK10, TAOK3, YES1, | 31 |
| SB1-G-190 | 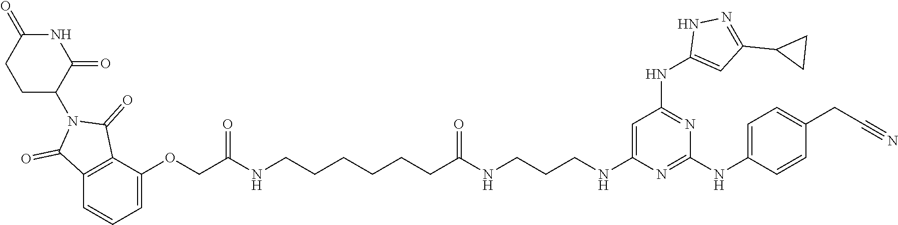 [SMILES: N#CCc1ccc(Nc2nc(NCCCNC(=O)CCCCCCNC(=O)COc3cccc4c3C(=O)N(C3CCC(=O)NC3=O)C4=O)cc(Nc3cc(C4CC4)n[nH]3)n2)cc1] | AAK1, AURKA, BMP2K, GSK34, GSK3B | 5 |
| SB1-G-192- 1 | 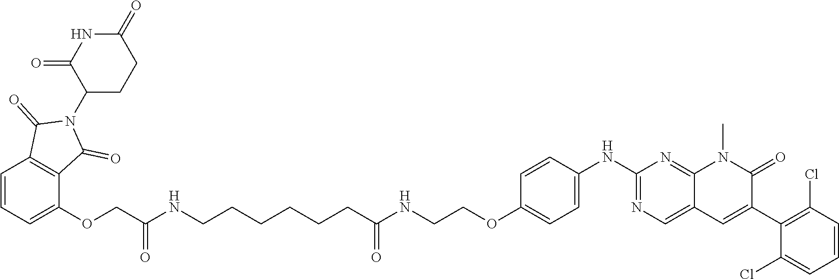 [SMILES: Cn1c(=O)c(-c2c(Cl)cccc2Cl)cc2cnc(Nc3ccc(OCCNC(=O)CCCCCCNC(=O)COc4cccc5c4C(=O)N(C4CCC(=O)NC4=O)C5=O)cc3)nc21] | ABL1, ABL2, AURKA, BLK, CSK, EPHA3, EPHB6, FYN, GAK, LCK, LIMK2, MAPK14, NLK, PDK1, PKMYT1, SIK2, SRC, TNK2, WEE1, YES1 | 20 |
| SB1-G-194 | 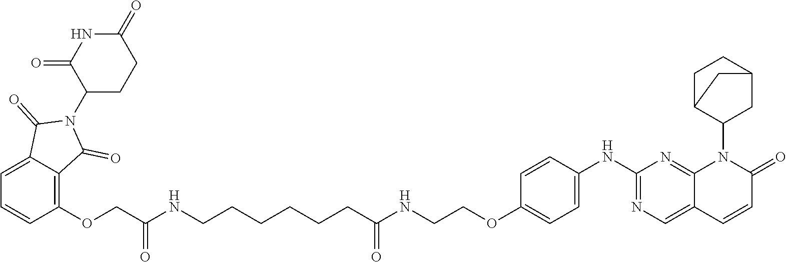 [SMILES: O=C(CCCCCCNC(=O)COc1cccc2c1C(=O)N(C1CCC(=O)NC1=O)C2=O)NCCOc1ccc(Nc2ncc3ccc(=O)n(C4CC5CCC4C5)c3n2)cc1] | AAK1, AURKA, AURKB, CDK6, CDK9, FGR2, STK17A, TTK | 8 |
| SB1-G-200 | 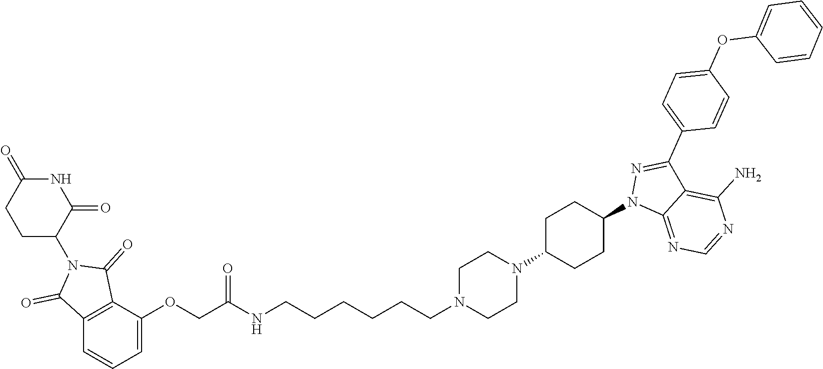 [SMILES: Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1] | ABL1, ABL2, BLK, CSNK1E, CSK, FYN, LATS1, LCK, LIMK1, MAP2K5, SRC | 11 |
| SK-3-87 | 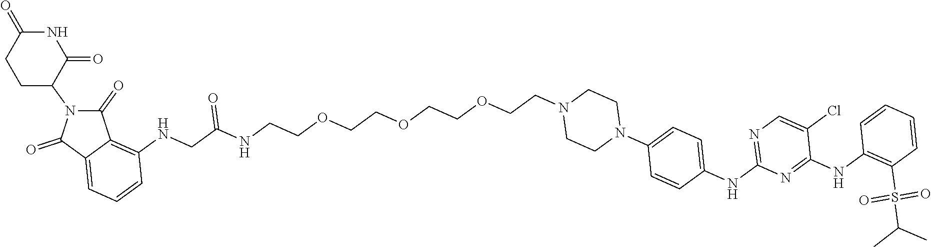 [SMILES: CC(C)S(=O)(=O)c1ccccc1Nc1nc(Nc2ccc(N3CCN(CCOCCOCCOCCNC(=O)CNc4cccc5c4C(=O)N(C4CCC(=O)NC4=O)C5=O)CC3)cc2)ncc1Cl] | AAK1, AURKA, BLK, CDK12, CDK17, CDK2, CDK5, CDK7, CDK9, FER, ITK, LCK, LIMK2, PTK2B, WEE1 | 15 |
| SK-3-89 | 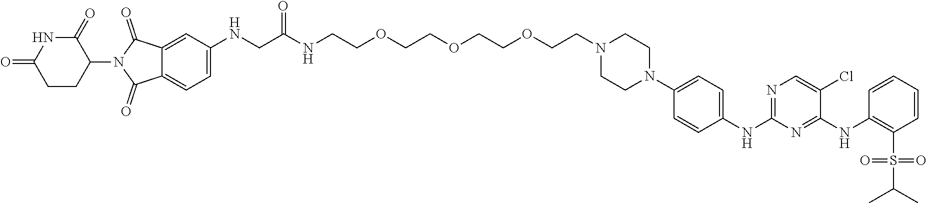 [SMILES: CC(C)S(=O)(=O)c1ccccc1Nc1nc(Nc2ccc(N3CCN(CCOCCOCCOCCNC(=O)CNc4ccc5c(c4)C(=O)N(C4CCC(=O)NC4=O)C5=O)CC3)cc2)ncc1Cl] | AAK1, AURKA, BLK, CDK12, CDK17, CDK2, CDK4, CDK5, CDK6, CDK7, CDK9, FER, ITK, LCK, LIMK2, PTK2B, STK10, WEE1 | 18 |
| SK-3-91 | 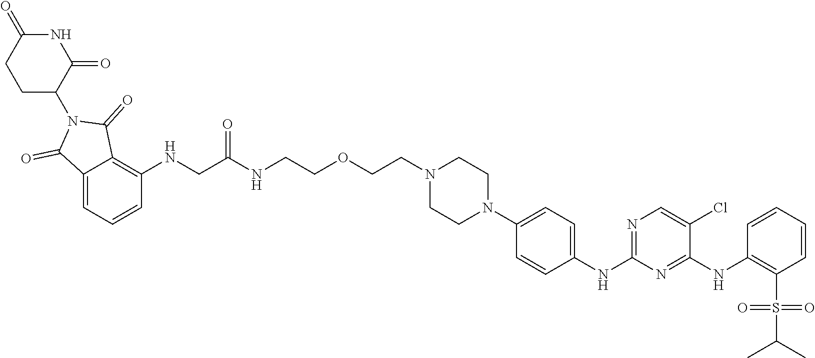 [SMILES: CC(C)S(=O)(=O)c1ccccc1Nc1nc(Nc2ccc(N3CCN(CCOCCNC(=O)CNc4cccc5c4C(=O)N(C4CCC(=O)NC4=O)C5=O)CC3)cc2)ncc1Cl] | AAK1, ABL1, ABL2, AKT2, AKT3, AURKA, AURKB, BCKDK, BLK, BMP2K, BMPR1A, BUB1, BUB1B, CDC7, CDK10, CDK12, CDK13, CDK14, CDK16, CDK17, CDK18, CDK2, CDK4, CDK5, CDK6, CDK7, CDK9, CO18A, CSK, CSNK1D, EPHB2, EPHB4, FER, FYN, GAK HIPK1, ITK, LATS1, | 89 |
| | | LCK, LIMK1, LIMK2, LRRK2, MAP3K1, MAP3K11, MAP3K12, MAP3K21, MAP4K1, MAP4K3, MAPK6, MAPK7, MARK2, MARK4, MAST3, MKNK2, NEK2, PDK3, PLK1, PLK4, PRAG1, PRKAA1, PRKAA2, PTK2, PTK2B, PTK6, RIOK2, RPS6KA1, RPS6KA6, RPS6KB1, RPS6KC1, SBK1, SIK2, SRC, STK17A, STK17B, STK32C, STK33, STK40, TEC, TGFBR1, TNK1, TNK2, TRIB3, TRPM7, TTK, UHMK1, ULK1, ULK3, WEE1, YES1 | |
| SK-3-93 | 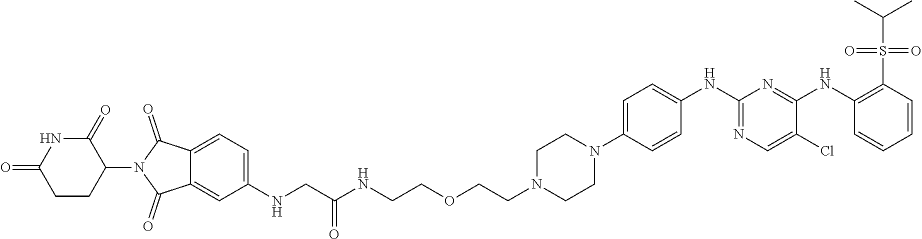 [SMILES: CC(C)S(=O)(=O)c1ccccc1Nc1nc(Nc2ccc(N3CCN(CCOCCNC(=O)CNc4ccc5c(c4)C(=O)N(C4CCC(=O)NC4=O)C5=O)CC3)cc2)ncc1Cl] | AAK1, ABL2, AURKA, BLK, BMP2K, CDK12, CDK13, CDK17, CDK2, CDK4, CDK5, CDK6, CDK7, CDK9, FER, GAK, ITK, LCK, LIMK2, PRKAA1, PTK2B, RPS6KA1, SRC, WEE1, | 24 |
| TL12-186 | 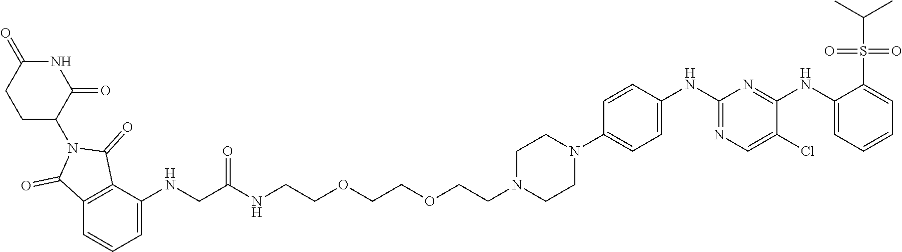 [SMILES: CC(C)S(=O)(=O)c1ccccc1Nc1nc(Nc2ccc(N3CCN(CCOCCOCCNC(=O)CNc4cccc5c4C(=O)N(C4CCC(=O)NC4=O)C5=O)CC3)cc2)ncc1Cl] | AAK1, ABL1, AURKA, AURKB, BLK, BMPR1A, BUB1, BUB1B, CDK10, CDK12, CDK13, CDK17, CDK2, CDK4, CDK5, CDK6, CDK7, CDK9, COQ8A, COQ8B, CSNK1D, CSNK1E, | 60 |
| | | EEF2K, FER, FLT1, GAK, HASPIN, IRAK4, ITK, LCK, LIMK1, LIMK2, MAP3K11, MAP3K21, MAP4K1, MAP4K3, MAPK6, MAPK7, MARK2, MARK4, NEK2, NLK, PDK2, PLK4, PRAG1, PRKAA1, PTK2B, RIOK2, RPS6KA1, SIK2, SRC, STK10, STK17A, STK35, TEC, TNK2, ULK1, ULK3, WEE1, YES1 | |
| TL13-112 | 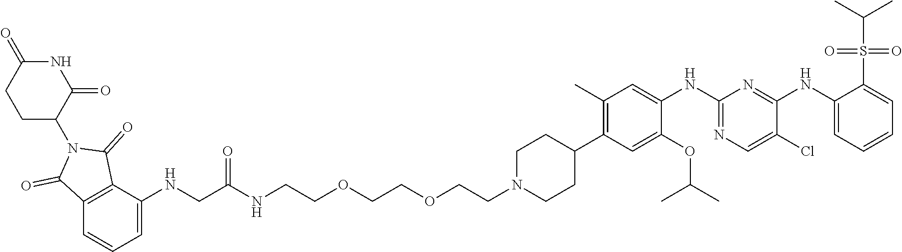 [SMILES: Cc1cc(Nc2ncc(Cl)c(Nc3ccccc3S(=O)(=O)C(C)C)n2)c(OC(C)C)cc1C1CCN(CCOCCOCCNC(=O)CNc2cccc3c2C(=O)N(C2CCC(=O)NC2=O)C3=O)CC1] | FER, PTK2, RPS6KA1 | 3 |
| TL13-117 | 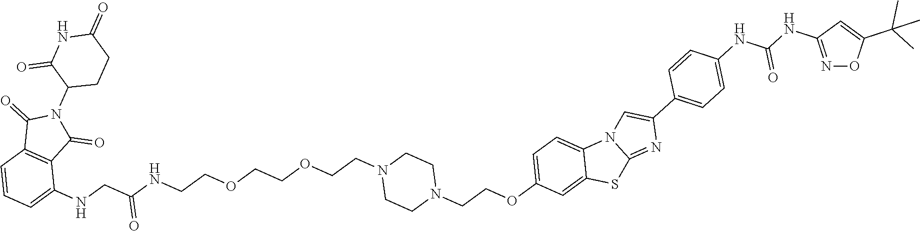 [SMILES: CC(C)(C)c1cc(NC(=O)Nc2ccc(-c3cn4c(n3)sc3cc(OCCN5CCN(CCOCCOCCNC(=O)CNc6cccc7c6C(=O)N(C6CCC(=O)NC6=O)C7=O)CC5)ccc34)cc2)no1] | NA | NA |
| TL13-12 | 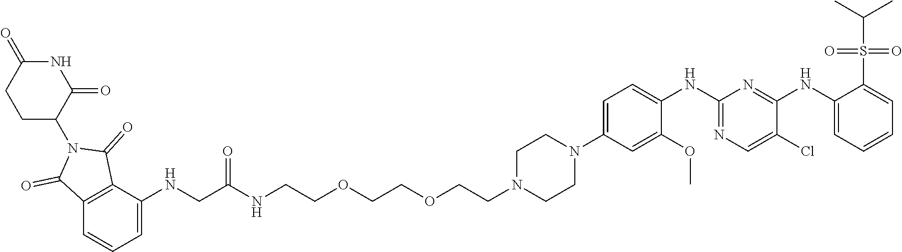 [SMILES: COc1cc(N2CCN(CCOCCOCCNC(=O)CNc3cccc4c3C(=O)N(C3CCC(=O)NC3=O)C4=O)CC2)ccc1Nc1ncc(Cl)c(Nc2ccccc2S(=O)(=O)C(C)C)n1] | AURKA, FER, PRK2 | 3 |
| TL13-150 | 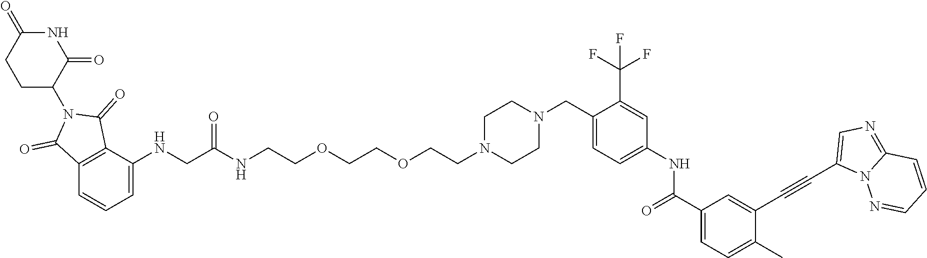 [SMILES: Cc1ccc(C(=O)Nc2ccc(CN3CCN(CCOCCOCCNC(=O)CNc4cccc5c4C(=O)N(C4CCC(=O)NC4=O)C5=O)CC3)c(C(F)(F)F)c2)cc1C#Cc1cnc2cccnn12] | BLK, CDK5, FER, GAK, IRAK1, LCK, MAP4K2, MAPK14, RIPK1, SRC | 10 |
| TL13-178 | 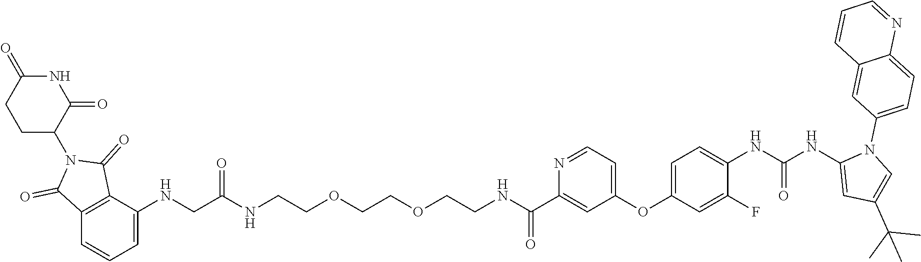 [SMILES: CC(C)(C)c1cc(NC(=O)Nc2ccc(Oc3ccnc(C(=O)NCCOCCOCCNC(=O)CNc4cccc5c4C(=O)N(C4CCC(=O)NC4=O)C5=O)c3)cc2F)n(-c2ccc3ncccc3c2)c1] | CDK4, LIMK1, MAP3K20, MAPK14, MAST3 | 5 |
| TL13-97 | 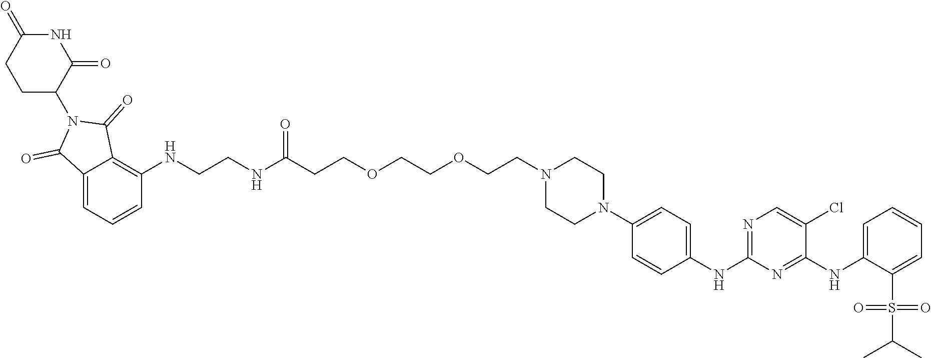 [SMILES: CC(C)S(=O)(=O)c1ccccc1Nc1nc(Nc2ccc(N3CCN(CCOCCOCCC(=O)NCCNc4cccc5c4C(=O)N(C4CCC(=O)NC4=O)C5=O)CC3)cc2)ncc1Cl] | AAK1, ABL2, AURKA, AURKB, BLK, BUB1B, CDK13, CDK17, CDK2, CDK4, CDK5, CDK6, CDK7, CDK9, FER, ITK, LCK, LIMK1, LIMK2, MAP3K11, MARK4, PLK4, PRKAA1, RPS6KA1, SRC, STK10, STK38, TEC, TNK2, ULK1, ULK3, WEE1 | 32 |
| TM-04-168- 03 | 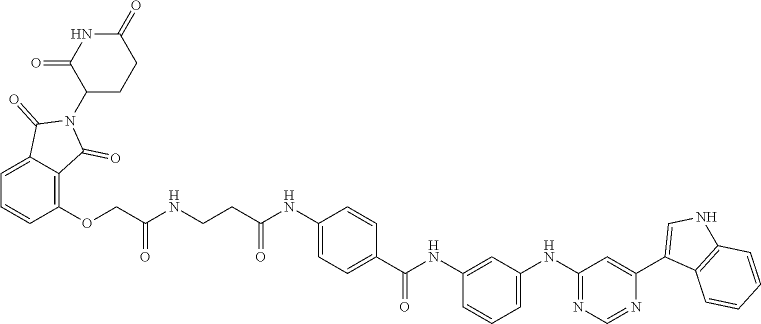 [SMILES: O=C(COc1cccc2c1C(=O)N(C1CCC(=O)NC1=O)C2=O)NCCC(=O)Nc1ccc(C(=O)Nc2cccc(Nc3cc(-c4c[nH]c5ccccc45)ncn3)c2)cc1] | NA | NA |
| TMX-01- 160-1 | 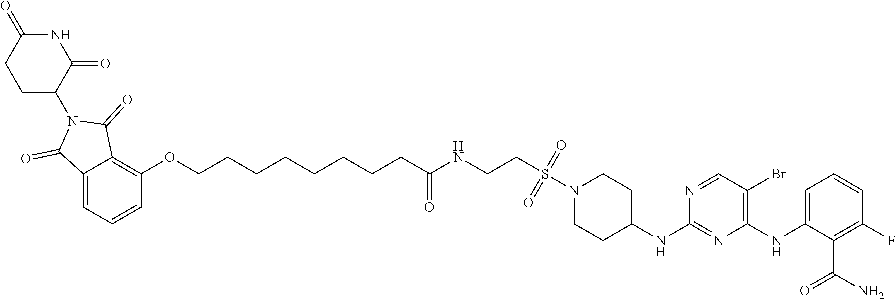 [SMILES: NC(=O)c1c(F)cccc1Nc1nc(NC2CCN(S(=O)(=O)CCNC(=O)CCCCCCCCOc3cccc4c3C(=O)N(C3CCC(=O)NC3=O)C4=O)CC2)ncc1Br] | AURKA, CDK1, CDK12, CDK16, CDK17, CDK2, CDK4, CDK5, CDK6, CDK7, CDK9, MAPK10, MAPK8, MAPK9, RPS6KA1 | 15 |
| TMX-02- 138 | 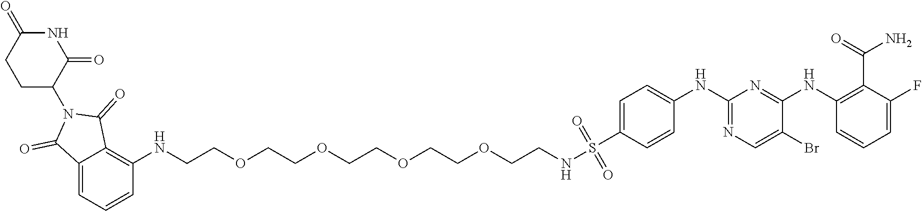 [SMILES: NC(=O)c1c(F)cccc1Nc1nc(Nc2ccc(S(=O)(=O)NCCOCCOCCOCCOCCNc3cccc4c3C(=O)N(C3CCC(=O)NC3=O)C4=O)cc2)ncc1Br] | AAKI, AURKA, AURKB, CDK1, CDK12, CDK16, CDK17, CDK2, CDK5, CDK7, CDK9, CLK1, FER, GSK3A, GSK3B, IRAK3, MAPSK11, MAPK8, MAPK9, NUAK1, PRKAA1, PRKAA2, PTK2B, RPS6KA1, RPS6KA3, STK33, STK38, TTK | 28 |
| TMX-02- 172 | 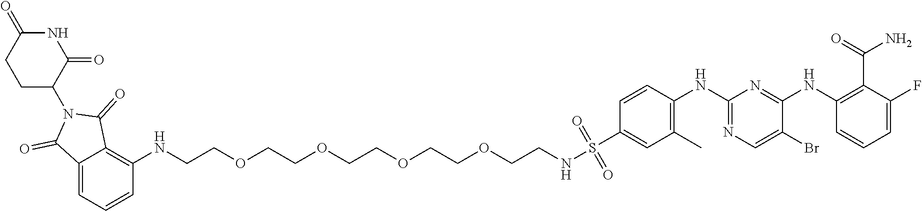 [SMILES: Cc1cc(S(=O)(=O)NCCOCCOCCOCCOCCNc2cccc3c2C(=O)N(C2CCC(=O)NC2=O)C3=O)ccc1Nc1ncc(Br)c(Nc2cccc(F)c2C(N)=O)n1] | AURKA, AURKB, CDK1, CDK2, CDK5, MAPK8, MAPK9, PTK2, RPS6KA1, RPS6KA3, STK33, TTK | 12 |
| TMX-02- 174 | 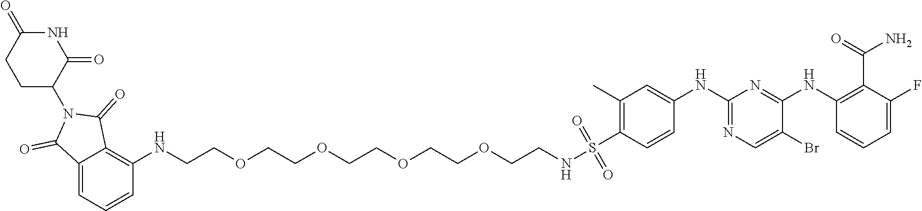 [SMILES: Cc1cc(Nc2ncc(Br)c(Nc3cccc(F)c3C(N)=O)n2)ccc1S(=O)(=O)NCCOCCOCCOCCOCCNc1cccc2c1C(=O)N(C1CCC(=O)NC1=O)C2=O] | AAK1, AURKA, AURKB, CDK1, CDK12, CDK16, CDK17, CDK2, CDK5, CDK7, CDK9, CLK1, FER, GSK3A, GSK3B, IRAK3, MAP3K11, MAPK8, MAPK9, NEK1, NEK3, NEK4, NEK9, NUAK1, PRKAA1, PRKAA2, PTK2, PTK2B, RPS6KA1, RPS6KA3, RPS6KA4, STK33, STK35, STK38, TBK1, TEC, TTK, WEE1 | 37 |
| WH-10417- 099 | 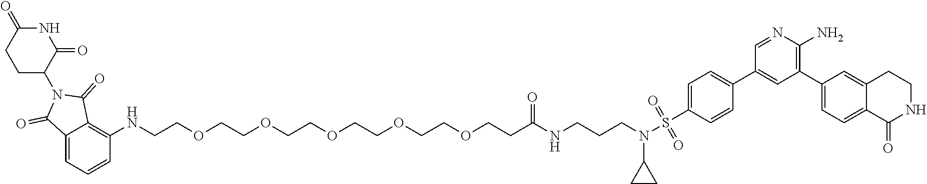 [SMILES: Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2)cc1-c1ccc2c(c1)CCNC2=O] | CDK14, CSNK1A1, CSNK1D, CSNK1E, GSK3A, GSK3B, LIMK2, MAP3K1, MINK1, NUAK1, PAK4, PIM2, STK10, STKI7B, STK35, STK4 | 13 |
| WH-9533- 153 | 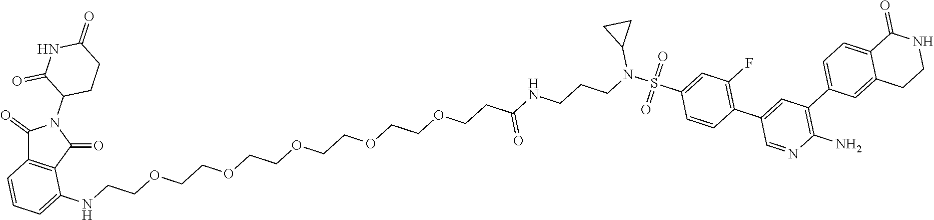 [SMILES: Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2F)cc1-c1ccc2c(c1)CCNC2=O] | CDK14, CSNK1D, GSK3A, GSK3B, NUAK1, PIM2, STK17B, STK35, STK4 | 13 |
| YKL-06- 102 | 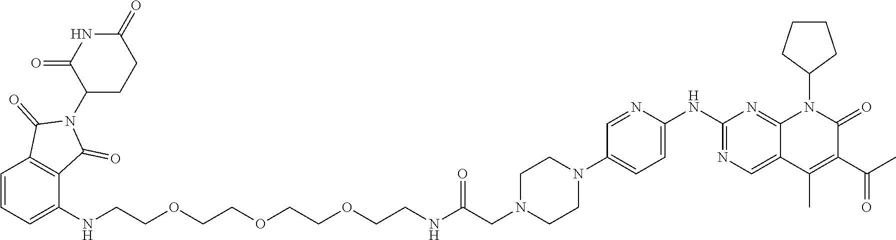 [SMILES: CC(=O)c1c(C)c2cnc(Nc3ccc(N4CCN(CC(=O)NCCOCCOCCOCCNc5cccc6c5C(=O)N(C5CCC(=O)NC5=O)C6=O)CC4)cn3)nc2n(C2CCCC2)c1=O] | CDK4, CDK6, WEE1 | 3 |
| ZNL-02-096 | 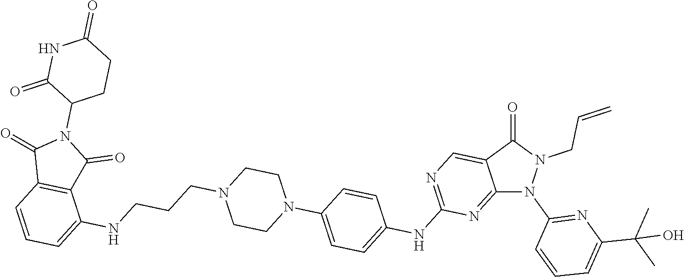 [SMILES: C=CCn1c(=O)c2cnc(Nc3ccc(N4CCN(CCCNc5cccc6c5C(=O)N(C5CCC(=O)NC5=O)C6=O)CC4)cc3)nc2n1-c1cccc(C(C)(C)O)n1] | COQ8A, MKNK2, PAK4, PDK1, PDK2, PDK3, PRKCI, TBCK, WEE1 | 9 |
| ZNL-03-127 | 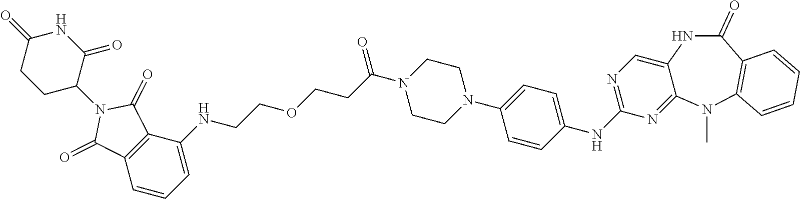 [SMILES: CN1c2ccccc2C(=O)Nc2cnc(Nc3ccc(N4CCN(C(=O)CCOCCNc5cccc6c5C(=O)N(C5CCC(=O)NC5=O)C6=O)CC4)cc3)nc21] | BLK LIMK1, LIMK2 STK17A, TNK2 | 5 |
| ZZ-1-31B | 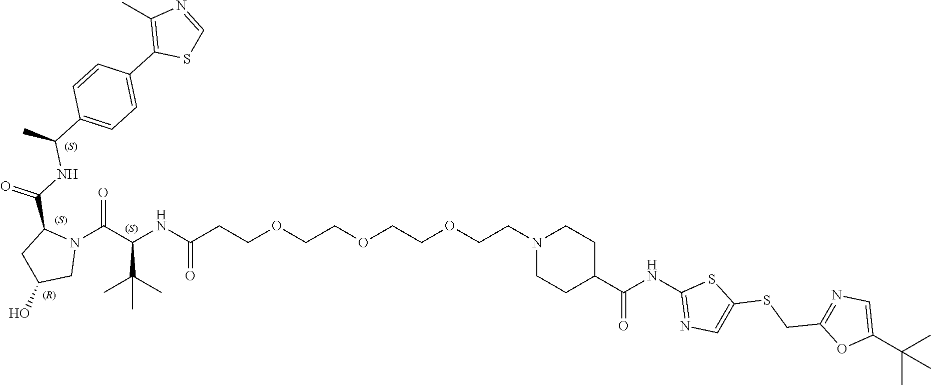 | CDK9, CLK1 | 2 |

A corresponding kinase hits list is set forth in Appendix I.

An extended kinome all hits list is set forth in Appendix II.

Degraders were prescreened for cellular permeability in the relevant CRBN or VHL target engagement (TE) assays and a final set of 91 compounds were selected for further characterization of their degradation targets using unbiased mass-spectrometry based global analysis, based on their chemical diversity and their ranking in cellular ligase engagement assays relative to close analogs (Table 1; FIG. 1B-FIG. 1C).

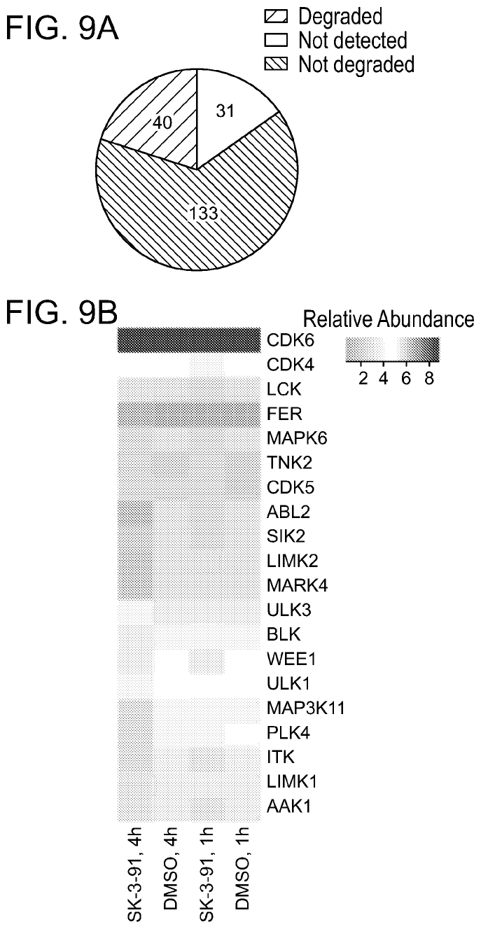

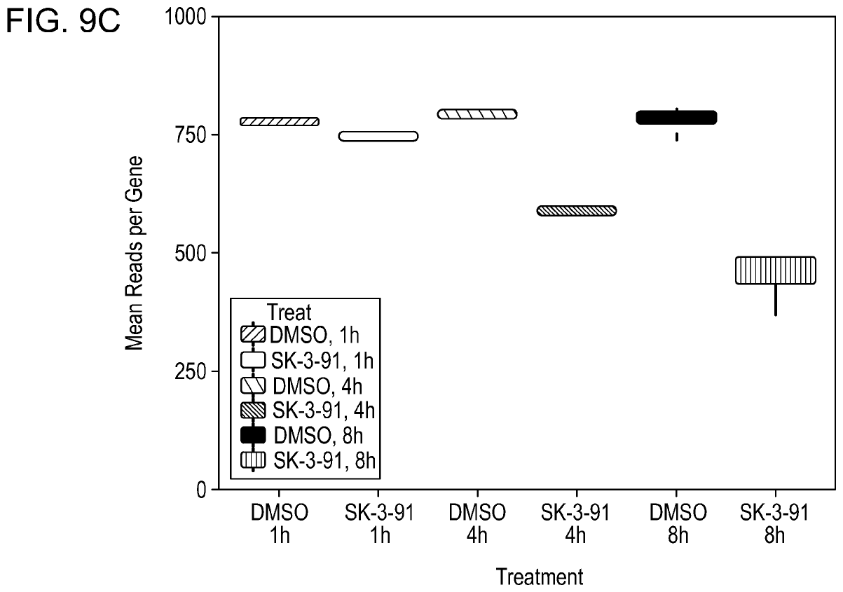

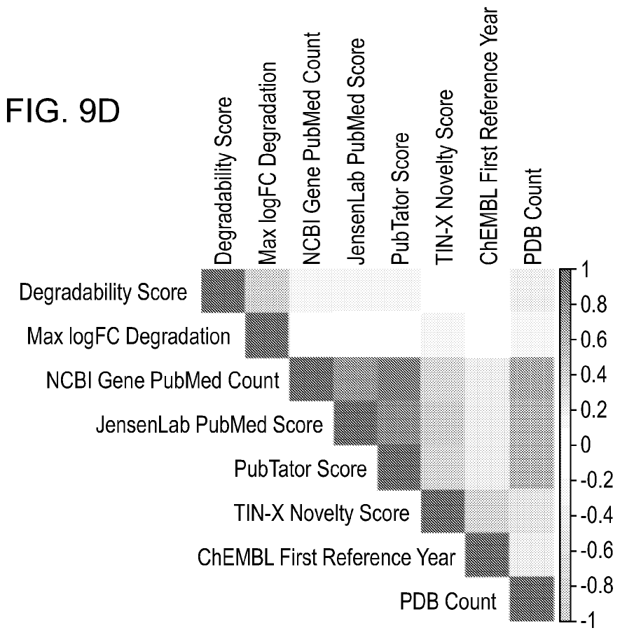

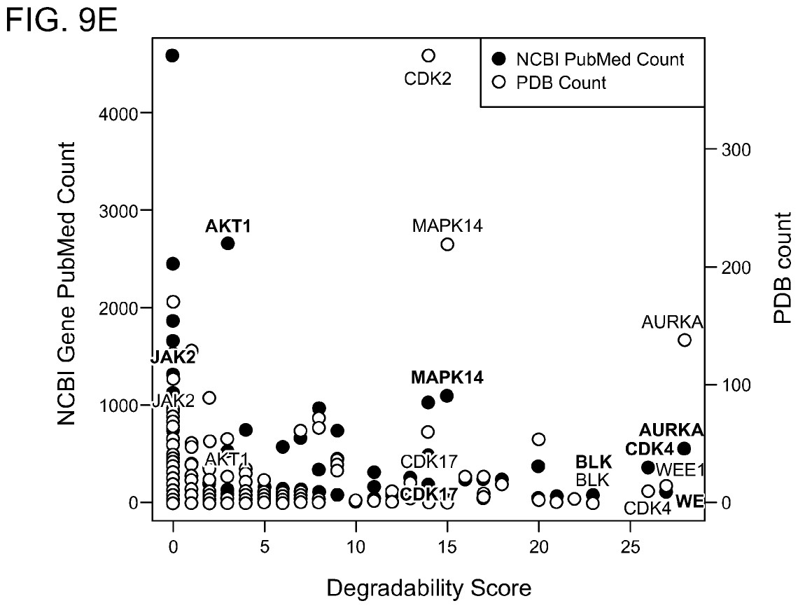

Whole cell multiplexed quantitative proteomics was employed to measure changes in abundance of the proteome in response to treatment with each of the kinase degrader molecules relative to DMSO (Table 1, Appendix I). Initial standard screening conditions of ≤8 h treatment time with 0.1-5 μM degrader compound were selected to reduce the likelihood of observing secondary effects on protein abundance and allow for similar comparisons (Bushman et al., Cell Chem Biol 28(1):78-87.e3 (2021)). Deep proteome coverage permitted quantification of 411 protein kinases across 7 cell lines: HEK293T, MOLT-4. Mino, MM1.S. OVCAR-8, KATO III and KELLY cells. To determine significantly downregulated targets, the abundance fold change cutoff was set at −1.25, and P-value<0.01, in order to allow detection of degradable kinases by unoptimized compounds at relatively short screening times of <8 h, 172 degraded protein-kinases were identified, corresponding to 33% of the human kinome, and 42% of the detected kinome (FIG. 1D, FIG. 1E: FIG. 8A-FIG. 8B; Table 1; Appendix I). An additional 204 proteins, that define the extended human kinome, were identified as kinase-like by sequence, structure, or annotation and include mitochondrial kinases, metabolic kinases which phosphorylate lipids, carbohydrates and nucleosides, and a subset of bromodomains (Moret el al., BioRxiv 10.1101:2020.2004.2002.022277 (2020)), 173 of these proteins were detected in at least 1 experiment, and degraders capable of inducing degradation of 40 proteins from this list were identified (Appendix II; FIG. 9A), validating them aspharmacologically related to protein kinases, and tractable TPD targets. In total, 212 degraded protein kinase or protein kinase-related targets were identified, a substantial increase from the kinases that have been reported to be targetable through degrader-induced mechanisms in the literature (FIG. 1F) (Bondeson el al., Cell Chem Biol 25:78-87.e75 (2018): Huang el al., Cell Chem Biol 25:88-99.e86 (2018)). Additionally, this dataset represents the first study that identifies kinases such as JAK2, CAMKK2 and DNAPK, that may be refractory to degradation using currently available TPD technologies, and characterizes their binary target engagement, ternary complex formation and expression profiles (Appendix I; Appendix II). The data reported herein quantify how different parental kinase-binder chemotypes affect degradation of individual kinases and will therefore serve as a valuable resource to support the decision making process for both tool compound and drug development pipelines.

A limitation of requirement for broad kinome coverage, extensive pharmacophore and binding mode representation, detectable cellular permeability, and whole proteome-based screening is that comprehensive synthesis and activity-based screening for all possible linker and E3-ligase recruiter analogs (>4,000 molecules) were not performed for each kinase binder series. Instead, focused SAR analysis coupled with proteomic profiling to interrogate these variables for a subset of highly multitargeted degraders.

### Assigning a Degradability Score

Empirical measures of target ligandability, a term that reflects the expected balance between effort and reward in a traditional small molecule inhibitor discovery project based on currently available technologies, have proved critical for target prioritization, and more recently, have enabled development of computational approaches to predict the ligandability of novel targets (Vukovic and Huggins, Drug Discov Today 23(6):1258-1266 (2018)). Although development of targeted protein degraders requires a POI binder, among already-liganded proteins such as kinases, it was hypothesized that different targets would have a different propensity for current approaches to degrader-mediated destruction, which was termed ‘degradability’. To assess degradability, the frequency of degradation (number of times a kinase is determined to be a down-regulated hit across the database) was determined for each protein kinase across all 154 treatments (Appendix I). It was rationalized that the probability of identifying the same kinase as a false positive in multiple treatments is low, therefore this analysis served to also assess the robustness of our data and subsequent interpretations. Across the 172 hits, 136 were shown to be downregulated in at least two independent treatments. Overall, 62 protein kinases were degraded in more than 10 independent treatments, and remarkably, 9 of these (CDK4, AURKA, FER, WEE1, BLK. LIMK2, CDK6, GAK, LIMK1) were each degraded in at least 40 of the 154 independent treatments, emphasizing their predisposition to induced degradation (FIG. 1G; Appendix I).

With long treatment times (>8 h), it is difficult to distinguish direct degradation of targets from down-regulation caused by downstream or secondary effects. To assess transcriptional changes in response to the most multi-targeted degrader molecule, SK-3-91, a time-course RNA-sequencing analysis experiment was performed, the transcript levels were found largely unchanged up until the 4-h time point (FIG. 9B), indicating that the hits in our database are unlikely to be transcriptionally downregulated. By the 8- and 12-h time points, complete transcriptional collapse occurred (FIG. 9C), an unsurprising result given the number of kinases (including transcriptional kinases such as CDK9) that are down-regulated in response to SK-3-91, and these data were therefore excluded from our kinase degradation count and degradability scoring assessments (FIG. 1E: FIG. 1G).

Next, the frequency of degradation assessment was corrected for over-representation of molecules in the full dataset by omitting replicate profiling of compounds under different experimental conditions, to remove any bias. This allowed the calculation of “the degradability score”, defined as the number of times a particular kinase scored as a downregulated ‘hit’ across all unique compound treatments. Unsurprisingly, the top degradable kinases mirror those from the previous analysis (CDK4, AURKA, FER, WEE1, BLK), confirming that in sufficiently large datasets, even with over-representation of certain molecules, frequency of degradation is a good measure of general tractability.

Published literature was used to assess the degree to which the scoring underestimates kinase degradability, 52 of the 57 kinases with at least one active degrader reported were also identified as degradable (>90%, FIG. 1F). Of the 5 degradable kinases that were detected in at least one published experiment by proteomics, and not degraded by any molecules in described herein (ALK, CK2, MEK, MAPK13 and HER2), it was assessed that all could be explained by low frequency of detection of the target protein, and/or slow degradation kinetics of the reported molecule. For example, reported CK2 degraders were active only at the 24-h time point (Chen et al., Bioorg Chem 81:536-544 (2018)). ALK degraders based on TAE684 have been reported in the literature, however, the reported degraders show maximal degradation at the 16-h time point and little activity at 4 h (Powell et al., J Med Chem 61:4249-4255 (2018)). Furthermore, in the profiling experiments, ALK was detected by proteomics in only 6/154 compound treatments. Outliers such as ALK represent limitations of the study, and indicate that some detected but not degraded kinases may indeed be tractable under different experimental conditions.

Previous studies have often been restricted to either a specific target, or chemical series, which has precluded formulation of general conclusions. With a large dataset in hand, global features of protein degradation were investigated. Whether the degradable kinase hits were biased towards kinases that are well-studied was evaluated by examining the correlation between the frequency of degradation and the maximum observed protein abundance fold change for each kinase, with knowledge metrics such as the pubmed score (Pletscher-Frankild et al., Methods 74:83-89 (2015)) and the number of entries in the PDB (FIG. 1H: FIG. 9D-FIG. 9E). No correlation was found between these variables, consistent with evidence that kinase inhibitor pharmacophores display a high degree of polypharmacology (Karaman et al., Nat Biotechnol 26:127-132 (2008); Knight el al., Nat Rev Cancer 10:130-137 (2010)) and indicating that our degradable kinome dataset may prove a valuable resource for generating initial leads for the development of selective chemical tools for understudied kinases, a key goal of the National Institutes of Health (NIH) Illuminating the Druggable Genome initiative (Rodgers et al., Nat Rev Drug Discov 17:301-302 (2018)). For example, this analysis revealed the presence of active degraders for at least 16 of the NIH's understudied kinases, some of which may be highly degradable (FIG. 1I). For example, cyclin-dependent kinase 17 (CDK17) is degraded by 15 different degraders. At least two lead-like degrader molecules, DD-03-156 and DD-03-106, were identified to exhibit a strong preference for degrading CDK17, with DD-03-156 inducing potent and selective degradation of only CDK17 and LIMK2 (FIG. 1J).

The human kinome contains approximately 55 pseudokinases, which are kinases that lack catalytic phospho-transfer activity but often have important scaffolding functions, making them potentially attractive targets for degraders (Moret et al., BioRxiv 10.1101:2020.2004.2002.022277 (2020)). Out of 42 pseudokinases quantified, 10 were degradable by at least one compound in the set described herein, including well characterized pseudokinases IRAK3 and TRIB3 (FIG. 1I). Due to the increased interest in targeting lipid kinases for therapeutic applications (Burke, Mol Cell 71:653-673 (2018)), their degradability was examined, finding leads for putative cancer targets PI3K-γ. PIP5K1A, PIP4K2B and PIP4K2C (FIG. 1I). Together with the extended kinome analysis described herein (Appendix II), these data suggest a subset of therapeutically relevant non-protein kinases are tractable targets for TPD.

### Degradable Kinome Dataset Accelerates Lead Discovery

Beginning a targeted protein degrader discovery project with solid prior knowledge of optimal chemotype-target pairs can rapidly speed up hit-to-lead time (Brand et al., Cell Chem Biol 26:300-306, e309 (2019); Cromm el al., J Am Chem Soc 140(49):17019-17026 (2018); Dobrovolsky et al., Blood 133:952-961 (2019); Jiang et al., Angew Chem Int Ed Engl 58: 6321-6326 (2019); Li et al., Cell Chem Biol 27(1):57-65 (2019); Olson et al., Nat Chem Biol 14:163-170 (2018)). The design of bifunctional degrader molecules requires the choice of a target ligand and a ligase binder as well as careful consideration of linker length, exit vector and linker properties, all essential determinants for its effectiveness. Current degrader design for a selected target campaign starts with the identification of a high affinity target binding ligand followed by the synthesis of a library of molecules incorporating different ligase recruiters and linker lengths. The library of degraders is then screened for degradation activity, usually by western blot. While the number of reported successes in compound development might imply that the design of these molecules is seamless (Lebraud and Heightman, Essays Biochem 61:517-527 (2017)), some proteins have proven resistant to TPD, and most unsuccessful campaigns likely remain unpublished (Gasic et al., Cells 9(5):1083 (2020); Zeng et al., Cell Chem Biol 27(1):19-31 (2019)). Furthermore, candidate degrader compounds ‘unsuccessful’ against one target, often potently degrade another when subjected to unbiased proteome-wide screening.

To circumvent the initial struggles of small molecule degrader design, the chemo-proteomics data described herein provides critical insights regarding target tractability, and potential starting points for degraders against novel targets. Equally important and often overlooked, is the negative data contained within the dataset which illuminates the kinases that are not yet ‘degradable’ and reveals the chemical structures that are not active towards a particular kinase target (Table 1, Appendix I).

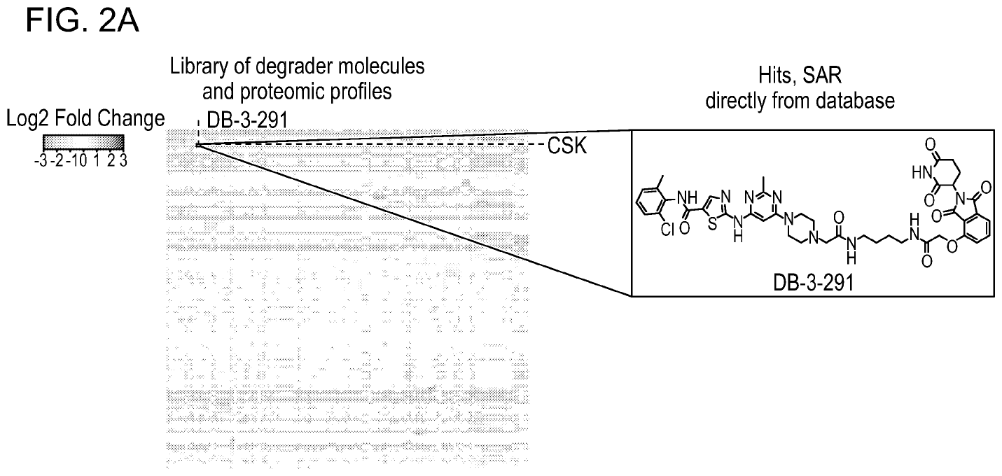

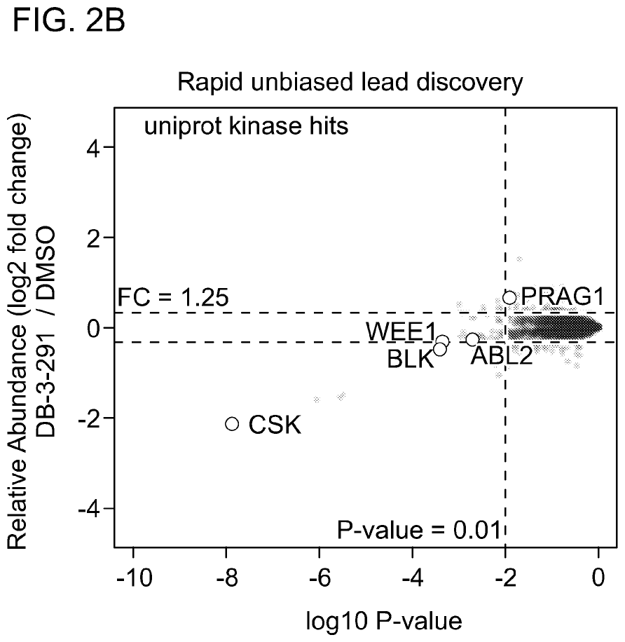

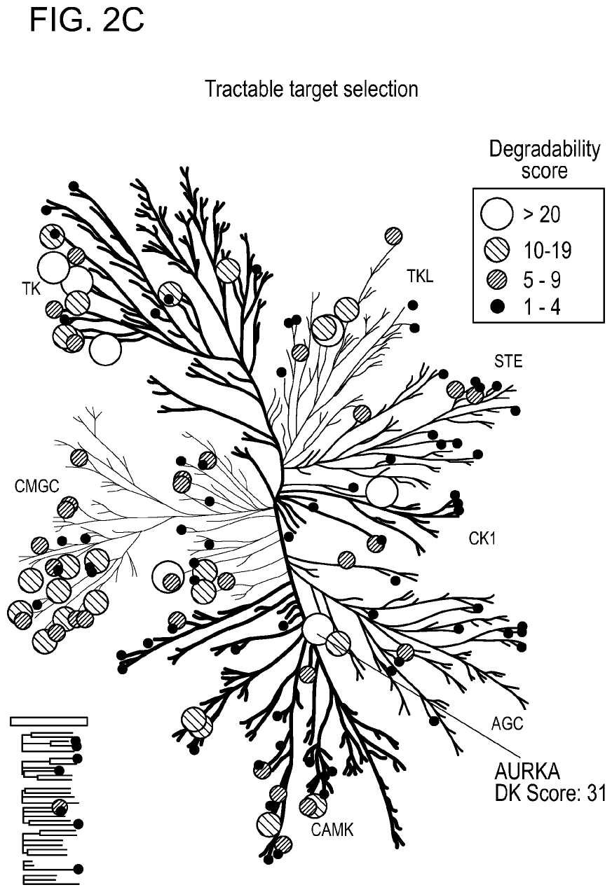

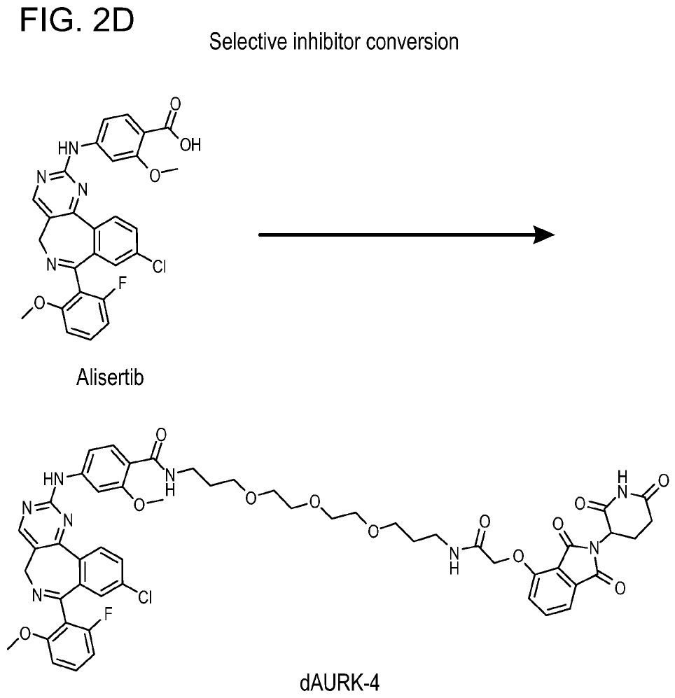

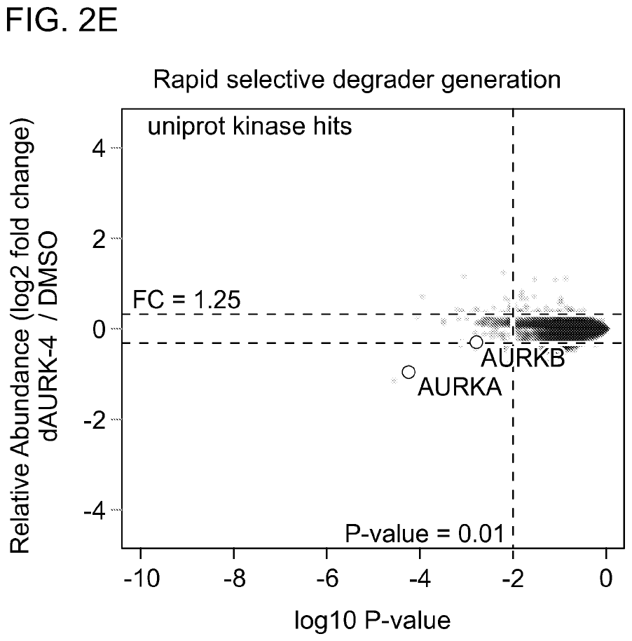

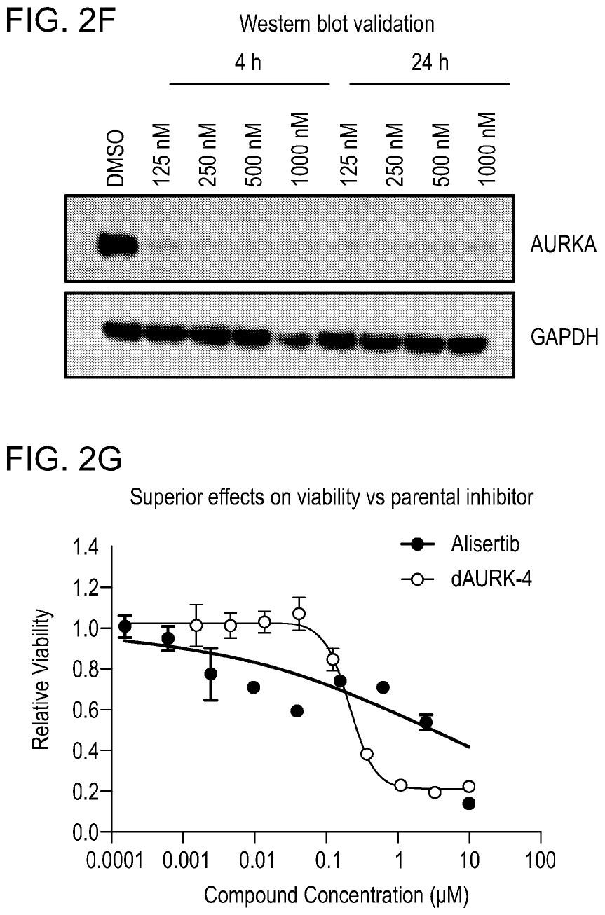

Two examples were used to illustrate the utility of database-assisted prioritization of lead molecules for novel kinase targets (FIG. 2A-FIG. 2G). To identify tractable targets, a list of degradable kinases (represented as heatmap in FIG. 2A) was created to evaluate the active molecules for lead-like selectivity profiles. Despite an absence of prior reports that CSK is a degradable kinase, 15 compounds in the library described herein were able to induce degradation of CSK. Compound DB-3-291 was found to induce the strongest degradation of CSK, in addition to having the greatest selectivity (FIG. 2A; FIG. 2B, Appendix I). The DB-3-291 degrader incorporates an immunomodulatory drug (IMiD) CRBN E3 ligase recruiter, an alkyl linker, and the multitargeted inhibitor dasatinib as the kinase binding ligand. Although the parental ligand was found to have a 1 nM in vitro binding affinity to CSK (KINOMEscan®), CSK was ranked 40th of over 100 kinases that had sub μM binding affinity (KD). Thus, it is surprising that this molecule does not degrade additional kinases (Davis et al., Nat Biotechnol 29:1046-1051 (2011)).

Given the observed selectivity profile of DB-3-291, a general lack of CSK selective inhibitors, and the role of this kinase in promoting the innate immune response to viral DNA, this molecule not only provides an advanced lead for further development into a chemical probe to interrogate CSK and STING signaling, but also demonstrates the utility of this resource for providing lead molecules for indications beyond oncology (Gao et al., Biochem Biophys Res Commun 526:199-205 (2020)). Whilst the inhibition profile of the kinase binder will likely contribute to phenotypic effects of selective degraders developed from multitargeted inhibitors at high doses, the substoichiometric and irreversible nature of degraders means that it is feasible to optimize degraders that effect complete target depletion at cellular concentrations well below those required for measurable target occupancy (Olson et al., Nat Chem Biol 14:163-170 (2018)).

Using a similar strategy, 31 molecules capable of inducing degradation of AURKA (Aurora A) were identified, indicating it is readily degradable by CRBN recruiting compounds (FIG. 2C; Appendix 1). Incorporation of an AURKA selective inhibitor, alisertib, as the target binder resulted in potent and selective degradation by dAURK-4, validated by immunoblotting, confirming the relative ease of active compound development for highly degradable kinases with reported selective ligands (FIG. 2C-FIG. F). Viability studies revealed that dAURK-4 has superior antiproliferative effects over parental inhibitor alisertib in the MM.1S multiple myeloma cell line (FIG. 2G). Mining the chemo-proteomics database described herein enabled rapid identification of degradable targets, and chemical starting points, significantly accelerating the development of effective kinase degrader molecules.

### Examining the Effect of Chemical and Cellular Variables on TPD Outcomes

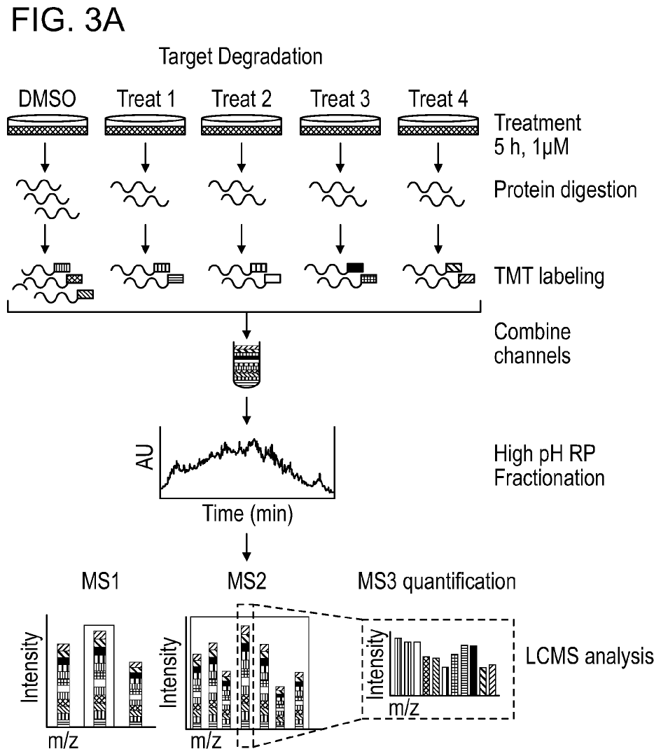

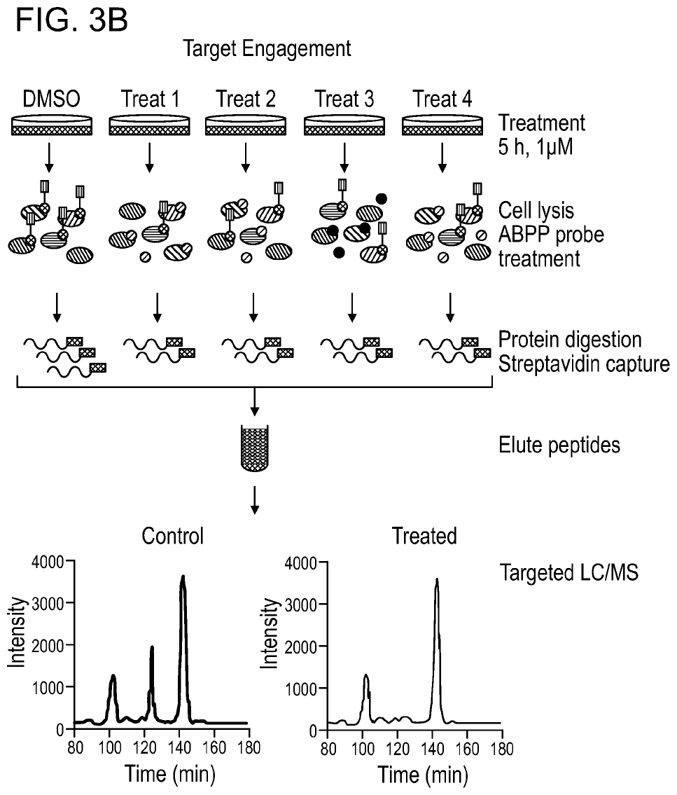

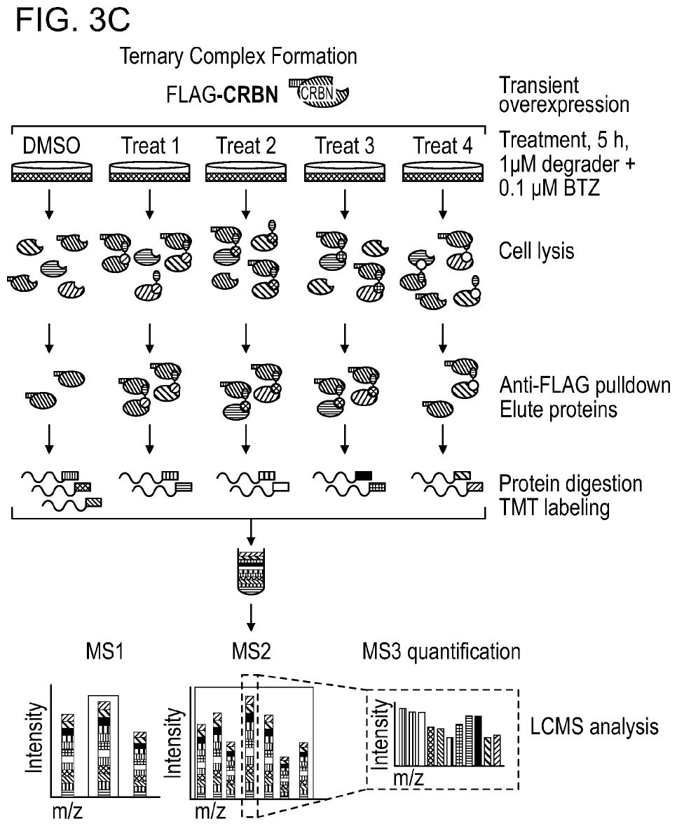

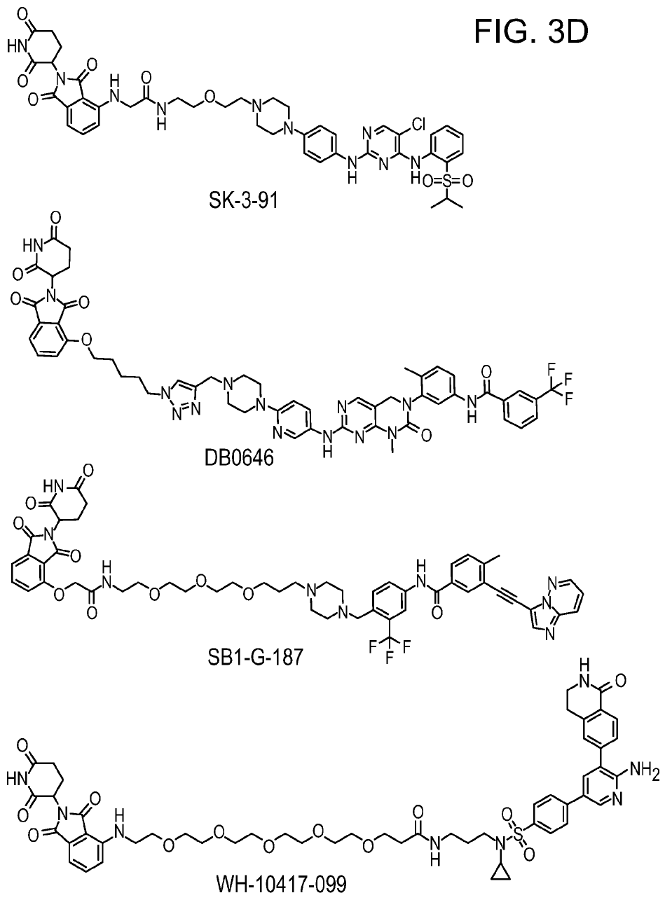

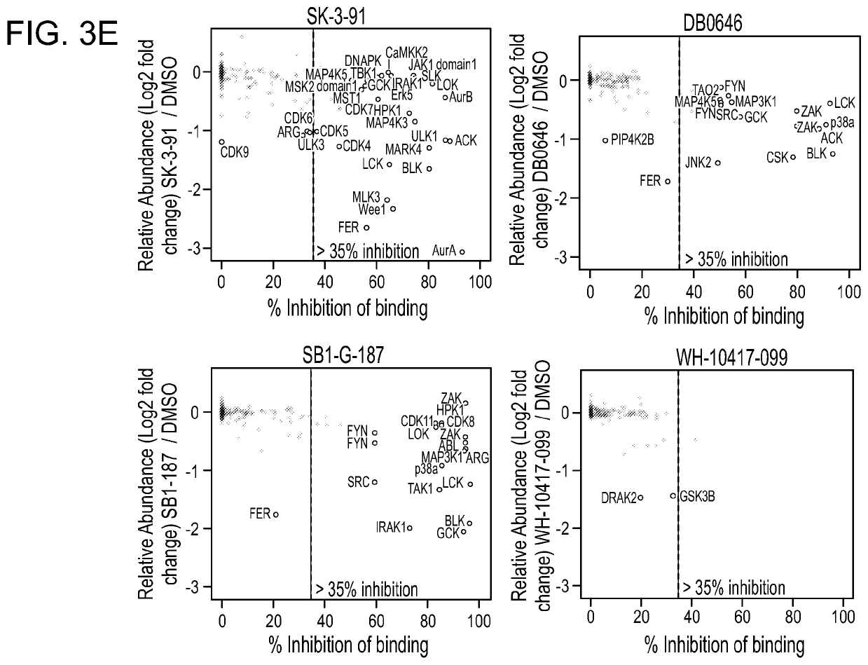

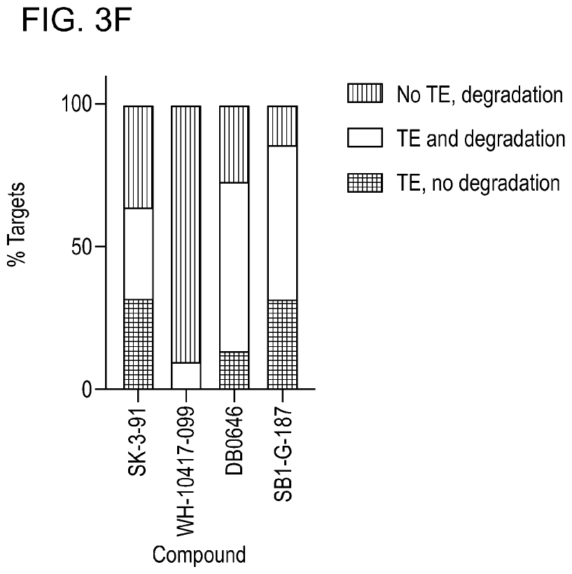

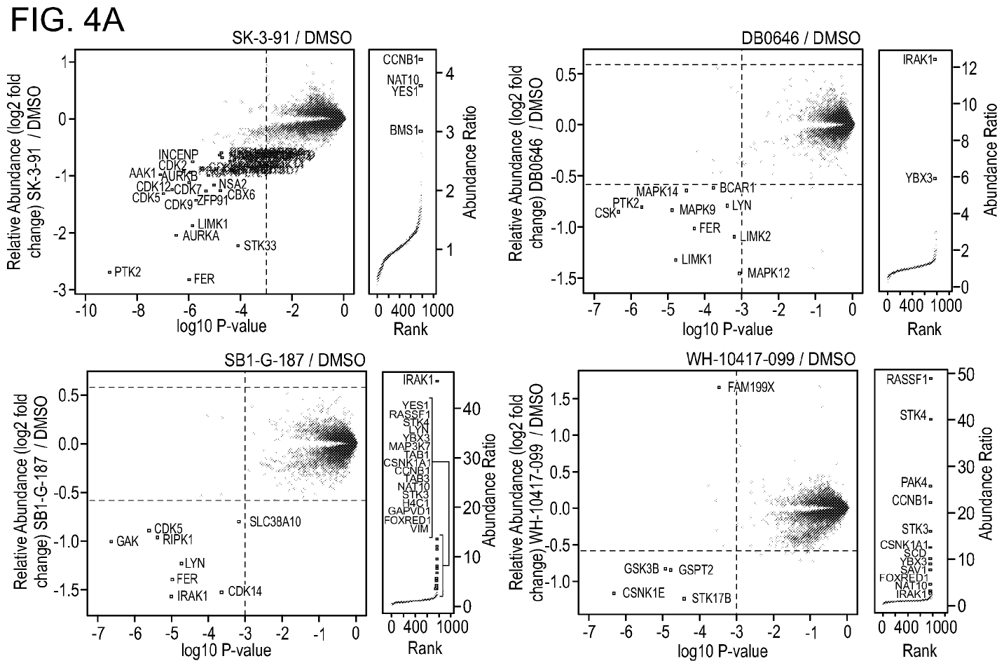

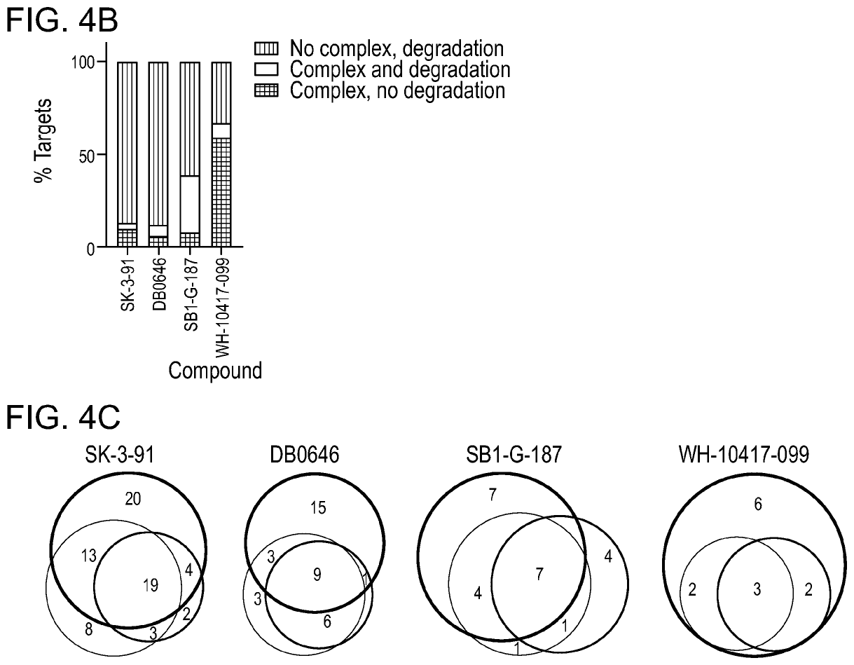

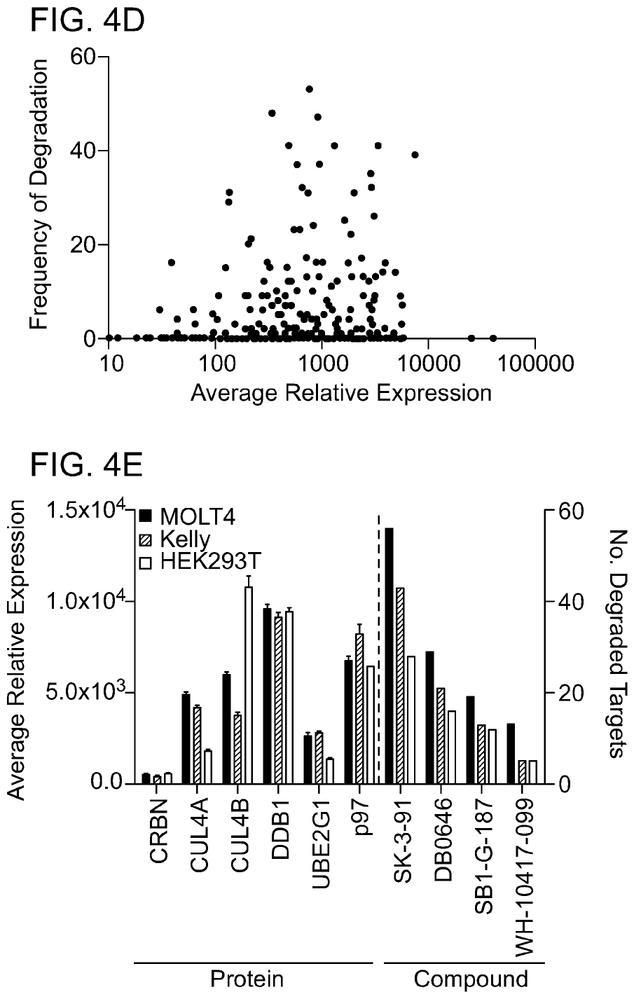

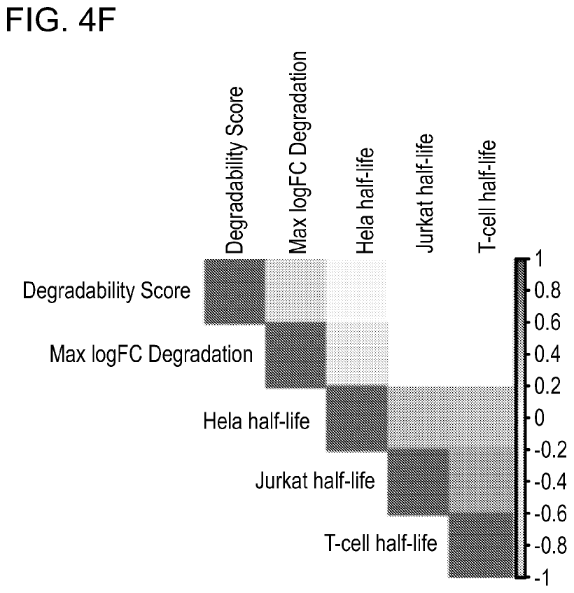

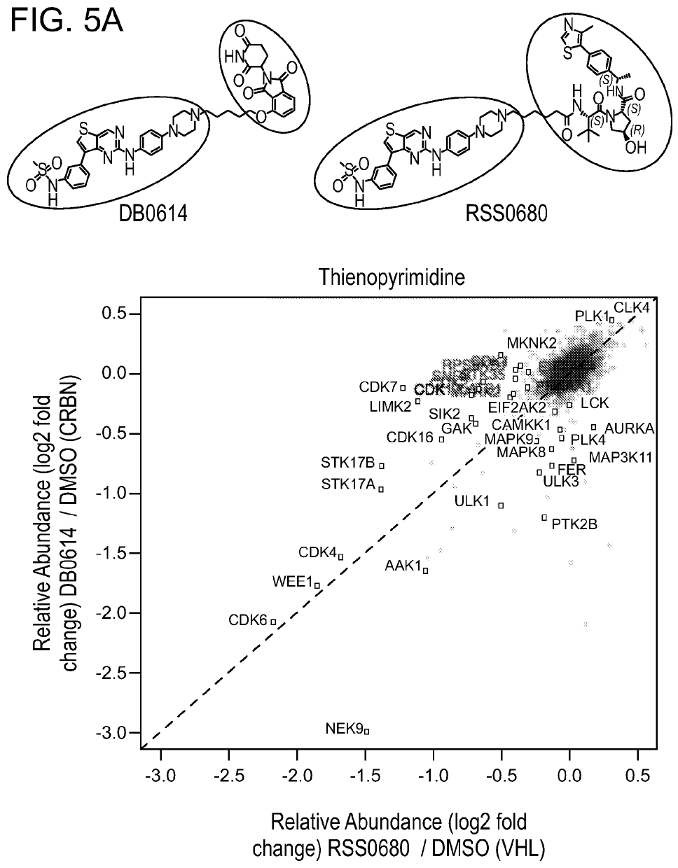

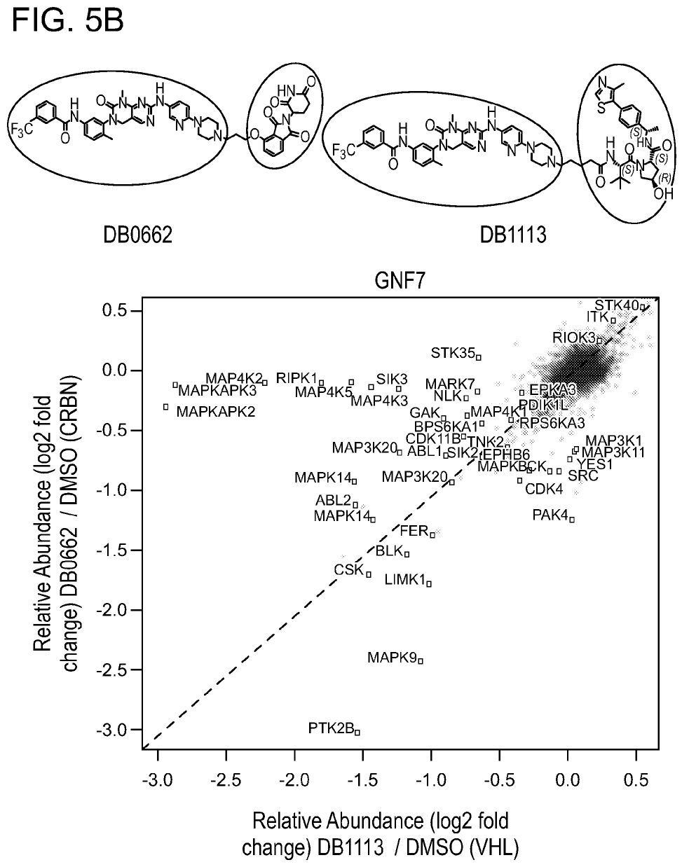

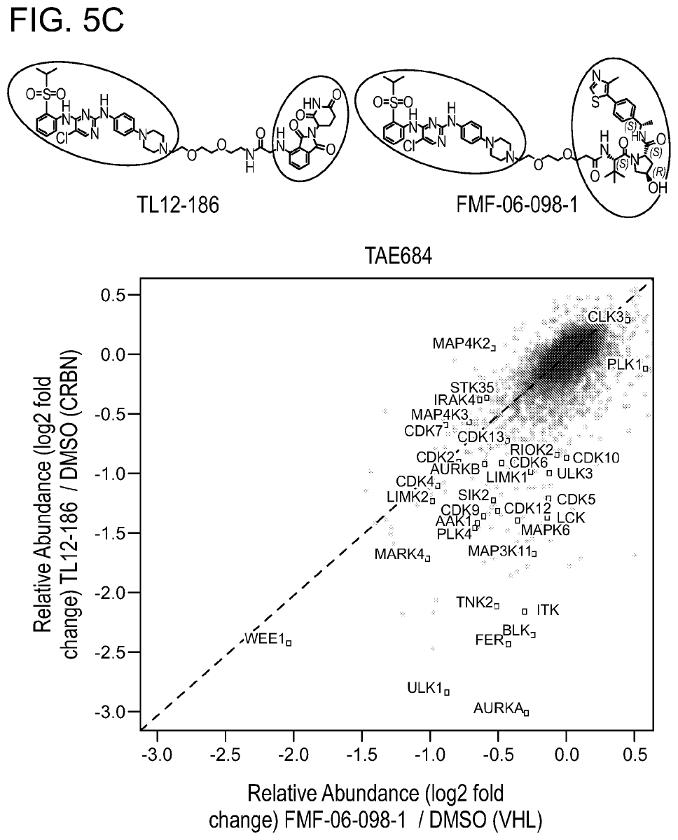

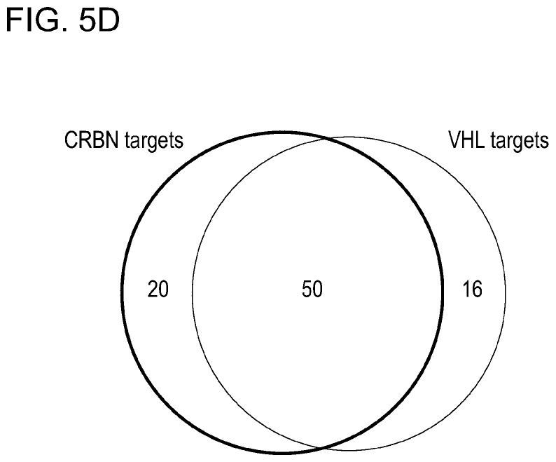

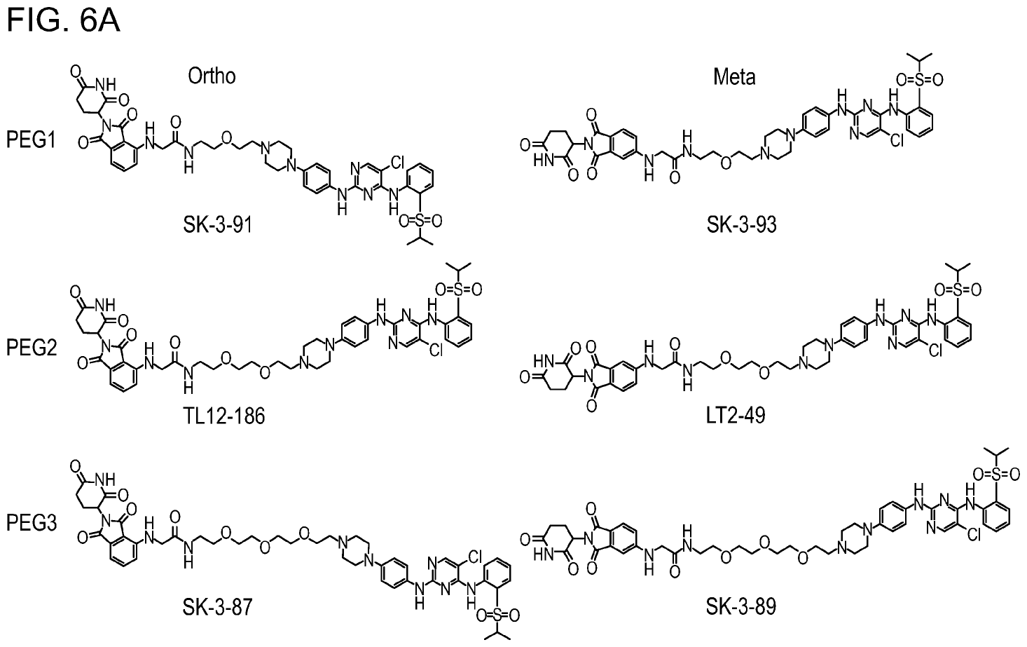

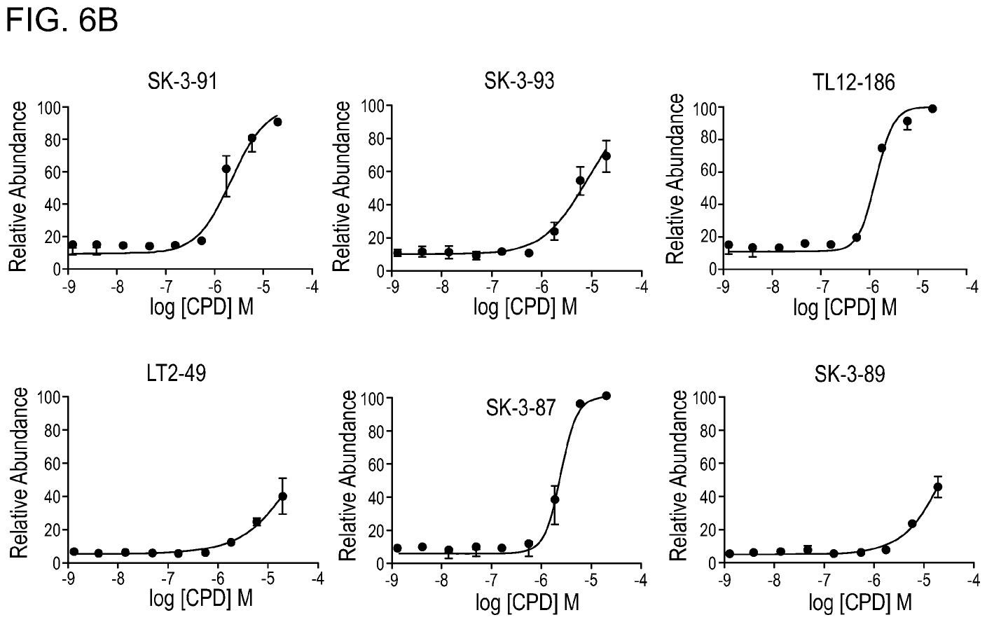

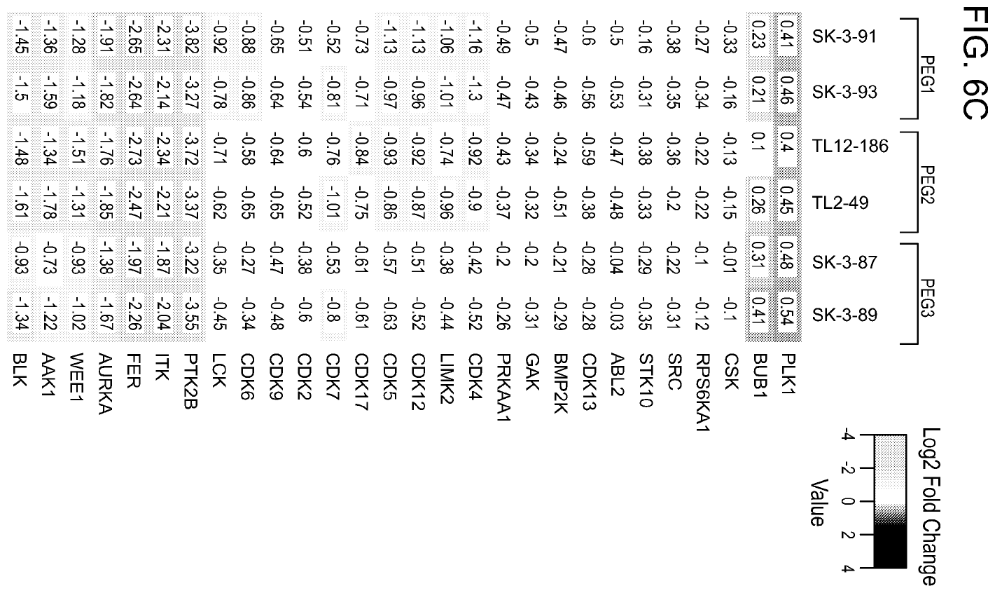

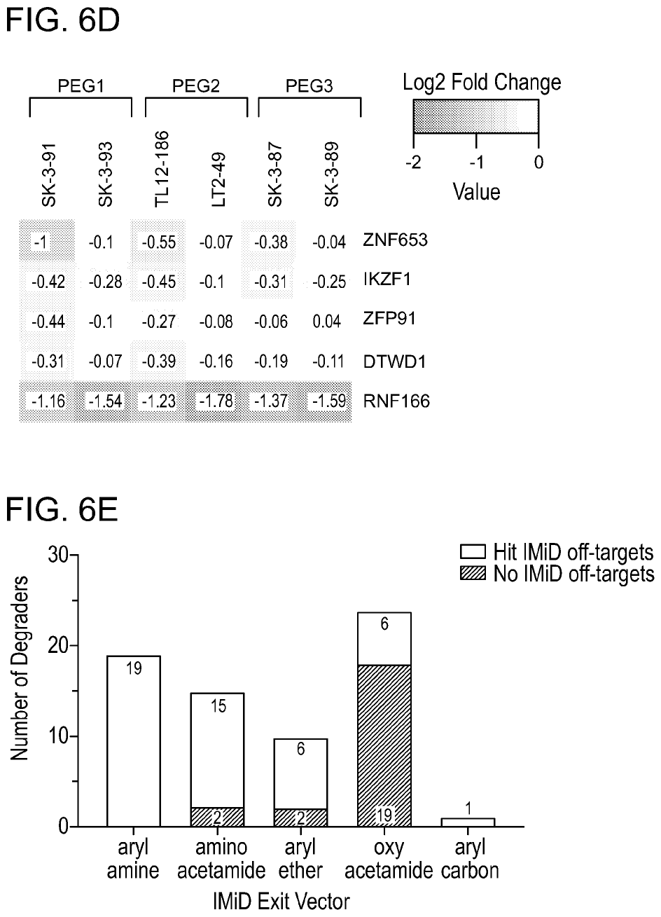

Having established tractable targets, the contributions that chemical and cellular variables have on TPD efficacy and selectivity kinome-wide were evaluated. Guidelines observations, oftentimes contradictory, have been reported for the optimization of degraders and summarized in a number of reviews (Churcher, J Med Chem 61(2):444-452 (2019); Paiva and Crews. Curr Opin Chem Biol 50:111-119 (2019)). However, as these studies are usually limited to one scaffold/binder, protein target and cell line combination, general conclusions about the frequency, magnitude and significance of these effects across a target space are challenging to extract. Motivated by a desire to understand the factors contributing to a successful TPD event, some of the field's new hypotheses kinome-wide were investigated. Herein, cellular events, including cellular target engagement (FIG. 3A-FIG. 3F), temary complex formation, target protein abundance, expression of components of the ubiquitin proteasome system (UPS) and ABC-drug transporters, target protein half-life, cell line variance (FIG. 4A-FIG. 4F), and the impact of altering the recruited E3-ligase (FIG. 5A-FIG. 5D), as well as chemical variables such as linker length and exit vector (FIG. 6A-FIG. 6E) were examined.

### Cellular Target Engagement Does Not Predict Degradation Efficiency

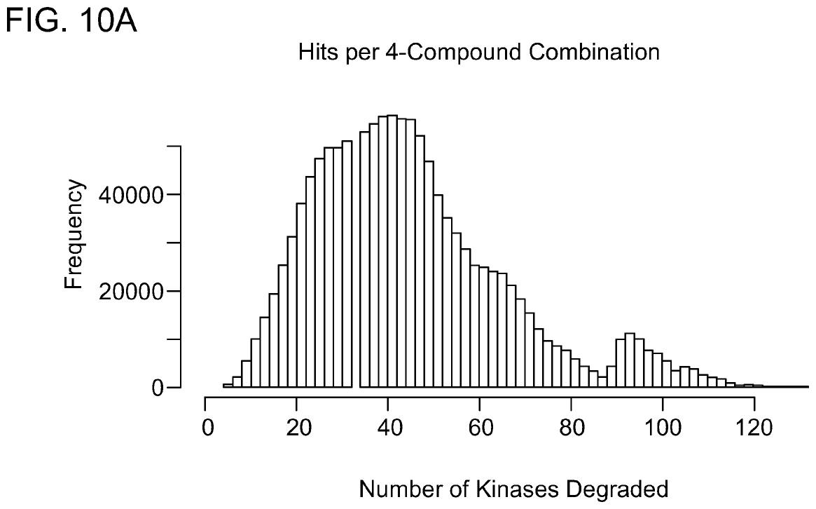

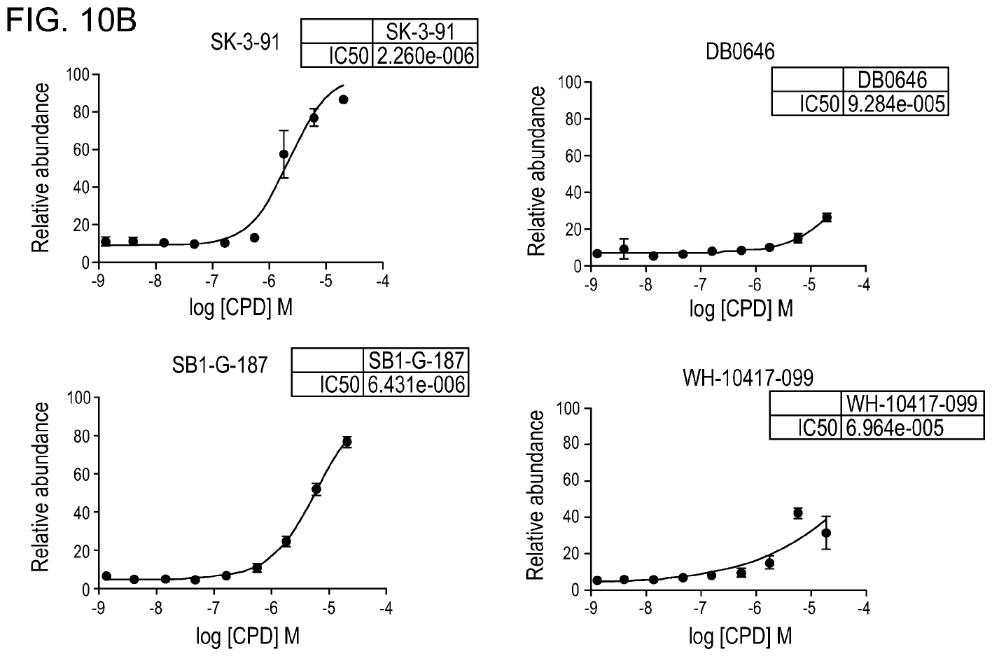

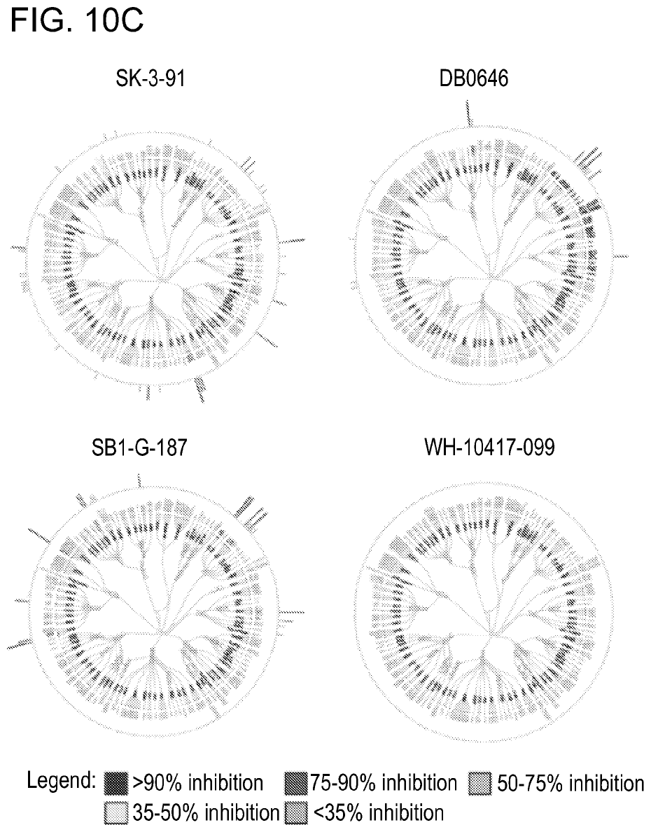

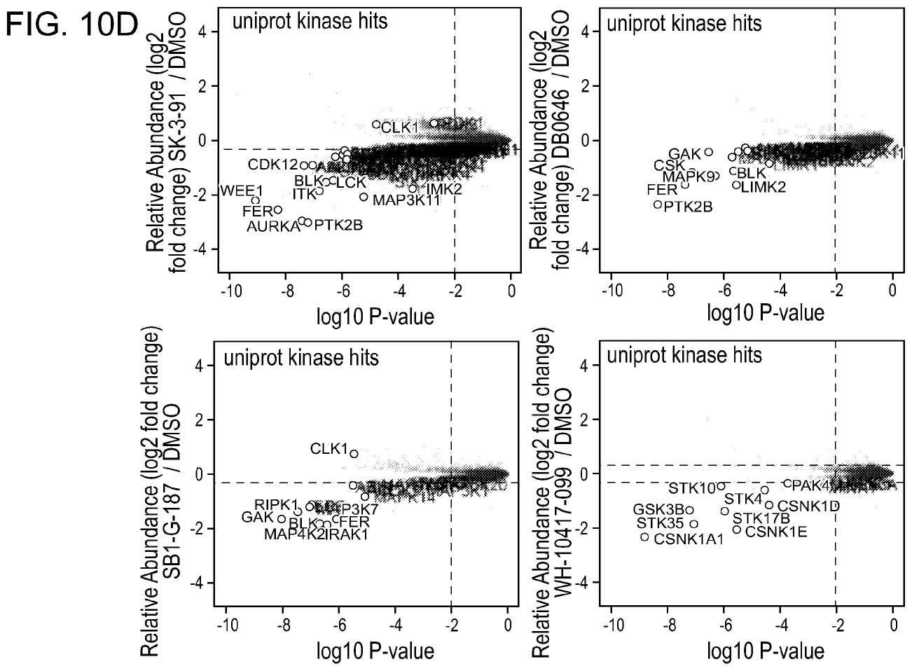

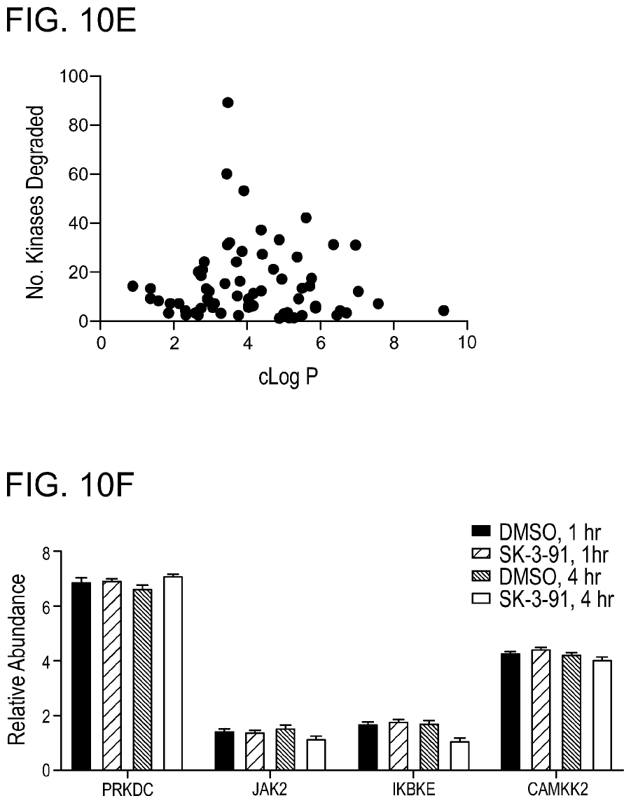

One of the key features of degraders is their potential for a sub-stoichiometric mode of action (Paiva and Crews, Curr Opin Chem Biol 50:111-119 (2019)). Together with the observation that ligase-degrader-POI ternary complexes can involve significant protein-protein contacts and even exhibit positive cooperative binding, it is widely believed that degraders uncouple efficacy from target occupancy (Gadd et al., Nat Chem Biol 13:514-521 (2017); Nowak et al., Nat Chem Biol 14:706-714 (2018); Olson et al., Nat Chem Biol 14:163-170 (2018); Roy et al., ACS Chem Biol 14:361-368 (2019)). While this has been confirmed in a limited number of individual studies, the generalizability of this hypothesis was tested across the kinome. To do so, the four degraders that could collectively in degradation of the largest number of unique kinases (SK-3-91, DB0646, SB1-G-187, and WH-10417-099, which together degrade>125 unique kinases) we selected (FIG. 3D. FIG. 10A). The focus of this test was to establish whether cellular target engagement (target occupancy) correlates with degrader efficacy. First cellular permeability of the four degraders using a cellular CRBN engagement assay was assessed to confirm that all four of these molecules are able to permeate the cell membrane and bind to the CRBN E3 ligase (FIG. 10B). Next, to measure the occupancy of kinase targets in live cells, KiNativ™ profiling in MOLT-4 CRBN−/− cells treated with 1 μM of each degrader (SK-3-91, DB0646, SB1-G-187, WH-10417-099) was performed for 5 h (FIG. 3B) (Patricelli et al., Biochemistry 46:350-358 (2007)). KiNativ™ is an activity based chemoproteomic assay, which measures the ability of a small molecule of interest to block the binding of a covalent ATP-mimetic probe. The resulting data revealed that of the ˜170 protein kinases quantified in both experiments. 47 were significantly engaged (>35% inhibition of binding) by at least one of the four multi-kinase targeting degraders (FIG. 10C). Comparison of the change in relative abundance of all quantified protein kinases (FIG. 10D) with their cellular binding affinities revealed that there is no correlation between cellular target engagement and potency of degradation across the four tested molecules (FIG. 3E), suggesting that target binding is not a major factor that drives efficacy of degradation. Furthermore, the proportion of degraded kinases with detectable binding varied dramatically from compound to compound, and was unrelated to cellular permeability (FIG. 3F).

However, instances were observed wherein a specific kinase is potently degraded with a high affinity degrader but shows no degradation with the weaker affinity molecules, suggesting that in order to be efficacious some degraders need to clear a certain threshold of binding affinity. For example, GCK is bound and degraded by DB0646 (58% I) and SB1-G-187 (94% I), but not degraded by SK-3-91 (50%1). Examples of kinases that were engaged by multiple degraders to a similar extent, but were degraded by only one of these molecules, such as IRAK1 and CDK17, were also observed (Appendix I).

Unlike kinase inhibitors, the clogP and the number of (degraded) targets of a molecule are not correlated across the dataset (FIG. 10E). Overall, the lack of correlation between target occupancy and degrader efficacy has important implications for degrader development, and shows how one can choose to develop degrader molecules whose pharmacology is driven by a combination of inhibition and degradation or where degradation is the primary driver. The empirical categorization of kinase and scaffold combinations into those where degradation efficacy is, and is not, affected by potency of binding may help determine if cellular target engagement (TE) should be incorporated into a compound optimization workflow.

There are many factors to consider when designing a degrader for a specific target, and w % ben a series is unsuccessful it is often difficult to gauge if this is in part because the target is particularly hard to degrade, and the size of the challenge if development is continued. Herein, the degradability score was used to identify four protein kinases (CAMKK2, DNAPK, IKKe, and JAK2) that, despite sufficient engagement by at least one molecule, show no indication of degradation by any of the 91 degraders included in our chemical library (Appendix I). To assess whether the absence of downregulation of protein levels could be a result of transcriptional compensation, the transcriptional changes of these four kinases was evaluated in response to a 4 h treatment with one of the multi-kinase targeting degraders. The resulting transcriptional analysis showed no upregulation of these kinases (FIG. 10F), ruling out compensatory upregulation through kinase engagement (FIG. 10C), and E3 ligase engagement (FIG. 10B) as causes of a lack of observed degradation.

Instead of relying on the binding profile of compounds to inform design, an activity-guided approach based on broad profiling data of multiple different scaffolds can accelerate lead identification for degraders.

### Formation of a Stable Temary Complex Does Not Predict Degradation Efficacy

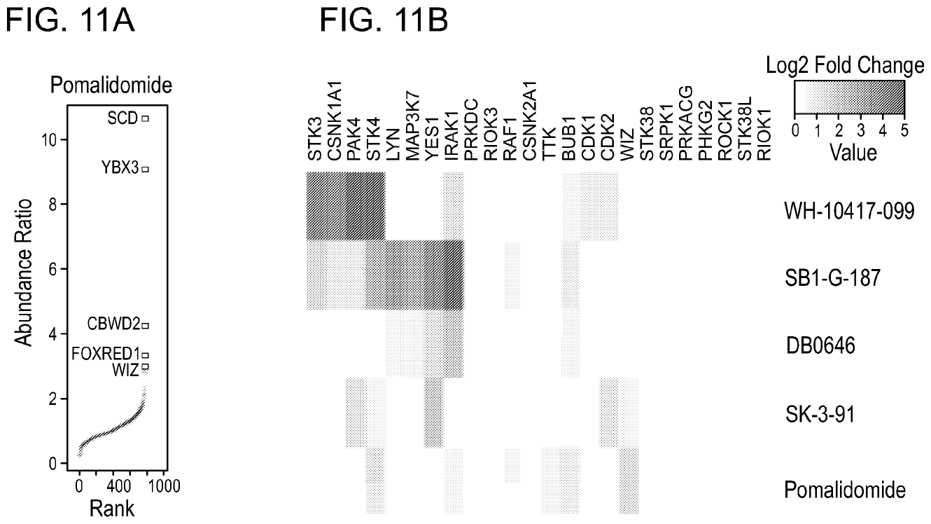

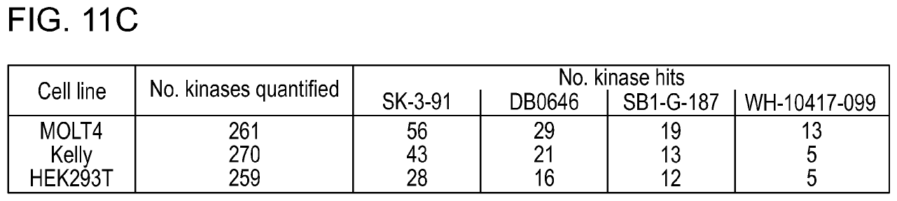

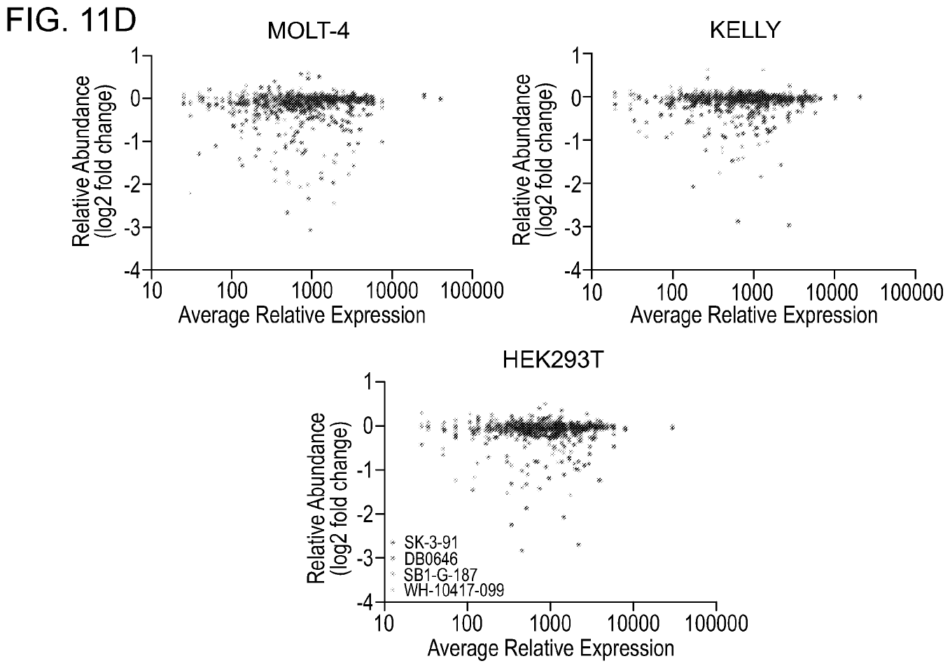

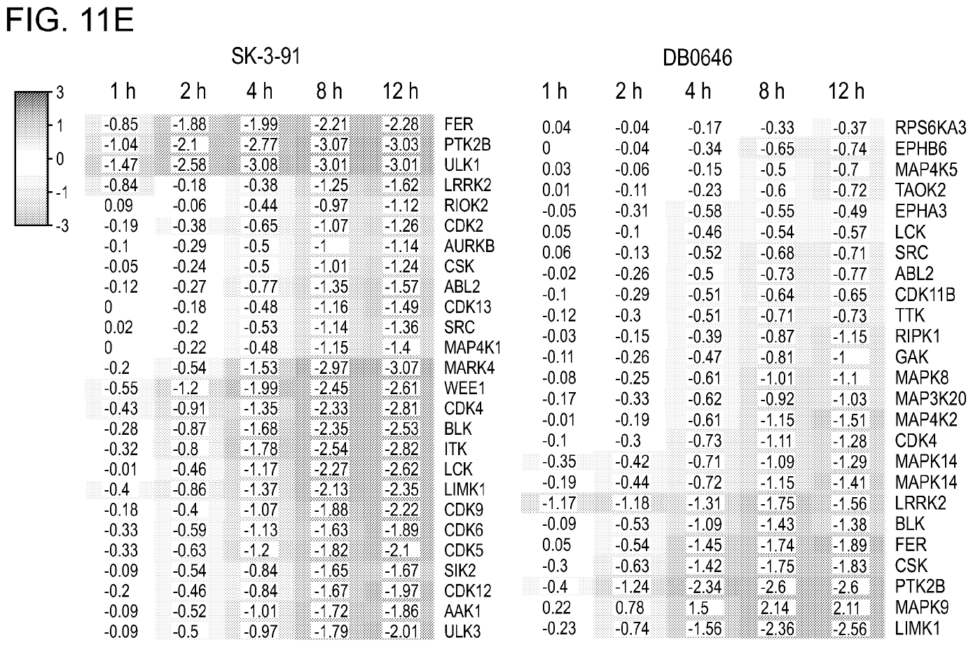

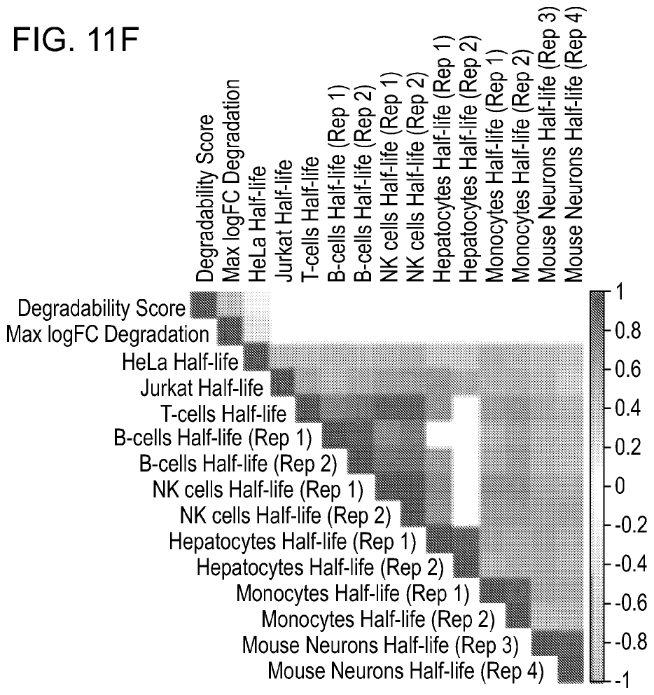

An important aspect of protein degraders is the multifaceted nature of the molecule that enables heightened target selectivity due to differences in complementarity of target-ligase interactions, which is not a consideration when assessing selectivity of inhibitors for target engagement (Famaby el al., Nat Chem Biol 15:672-680 (2019)). For compound-induced degradation to be successful, productive ternary complex formation (ligase-degrader-POI) is necessary for proximity-mediated ubiquitin transfer onto the POI. Multiple studies have reported that the stability of the E3-degrader-POI temary complex may influence degradation kinetics and selectivity, and may be a more reliable predictor for degradation than target engagement alone (Bondeson el al., Cell Chem Biol 25:78-87.e75 (2018); Roy et al., ACS Chem Biol 14:361-368 (2019)). To compare ternary complex formation to both target engagement and degradation across the kinome, and across multiple scaffolds, the breadth of kinases that form complexes with CRBN in the presence of our 4 selected multi-kinase targeting degraders (SK-3-91, DB0646, SB1-G-187, WH-10417-099; FIG. 4A-FIG. 4C, FIG. 11B) was experimentally assessed. Cellular affinity purification was performed, followed by mass spectrometry (AP-MS) of FLAG-tagged CRBN transiently overexpressed in HEK293T cells. FLAG-CRBN expressing cells were co-treated with proteosome inhibitor and 1 μM of each degrader for 5 h, and the degree of kinase target enrichment was compared to kinase degradation hits in matched global proteomics analysis experiments (FIG. 3A, FIG. 3C). The proteins identified as complexed with CRBN were enriched for kinases as well as their known binding partners such as Cyclin B (CDK1) and RASSF1 (STK4), consistent with the binding profiles of the assayed degraders. All kinases identified by AP-MS except CSNK1A1 were also detected in the HEK293T whole proteome degrader profiling experiment, allowing us to interrogate trends across 52 unique kinases (FIG. 4A; FIG. 11B). Limitations of this experiment include the potential loss of transient or weakly bound complexes in the enrichment and subsequent wash steps, the inherent noise associated with AP-MS relative to global proteomics analysis (Dunham et al., Proteomics 12:1576-1590 (2012): Yugandhar et al., Comput Struct Biotechnol J 17:805-811 (2019)) and the short time point of the experiment, which precludes detection of degradation events with slow kinetics. Therefore, the relationship between the formation of abundant, stable ternary complexes and rapid degradation was assessed.

Instances wherein a kinase enriched in the AP-MS experiment was degraded in the corresponding global proteomics profiling for every compound were found. However, overall, a low proportion of the degraded kinases for each molecule formed detectable ternary complexes in our experiment (FIG. 4B: Appendix I). For the two molecules with the highest number of degradation targets (DB0646, SK-3-91), fewer enriched proteins in the CRBN AP-MS were observed, relative to SB1-G-187 and WH-10417-099. It was hypothesized that this may be because these degraders form low levels of stable unique ternary complexes with multiple kinase partners, leading to less enrichment of any one kinase which dilutes the target enrichment to immeasurable levels, or these degraders form transient, unstable, but highly productive temary complexes that are unable to be captured in AP-MS experiments. In both interpretations, the rapid activity of DB0646 and SK-3-91 against their kinase targets is driven by their ability to induce more effective degradation catalysis, rather than induce higher levels of stable complex formation with their targets, relative to SB1-G-187 and WH-10417-099.

Although complex temary complex formation is a mechanistic requirement of TPD, the frequency with which effective complex formation results in productive degradation, is poorly understood. In this experiment, evidence of the formation of both productive and unproductive temary complexes with all compounds was observed (FIG. 4A: FIG. 11B). For YES1, IRAK1 and LYN, complex formation and degradation are sometimes detected together (YES1: DB0646, SB1-G-187. IRAK1, LYN: SB1-G-187), but complex formation does not predict degradation (YES1: SK-3-91, IRAK1: WH-10417-099). These data indicate that both kinases have high compatibility for degrader induced binding with CRBN, but that different complexes differ in their ability to efficiently catalyze degradation. Finally, it was observed that BUB1 was complexed but not degraded by all 4 degraders in HEK293T experiments, but was degraded in 24 independent treatments across the database in MOLT-4 and MM.1S cell lines, including by DB0646 and SK-3-91 (Appendix 1). Here, altered degradation kinetics or other cell-type related variables such as the presence and expression level of DUBs, or differences in post-translational modification of target lysines, could be drivers of this discrepancy. Together, these data highlight how the complex cellular environment, and the substoichiometric mode of action of degraders, decouple single molecular events, such as the degree of stable temary complex formation, from efficient degradation.

### Target Protein Abundance Does Not Predict Degrader Efficacy

The concentration of the two protein binding partners can affect ternary complex formation kinetics and equilibria in cells, and it has been suggested that target expression and/or local concentration influence target degradability (Sievers el al., Science 362(6414):eaat0572 (2018)). To investigate the dependence of target protein degradation efficiency on target expression level, the relative expression of proteins across three cell lines of different tissues of origin, MOLT-4, KELLY and HEK293T was quantitatively evaluated, and the four multi-kinase degraders in these lines were profiled profiled (FIG. 4C). Differences in the number of degraded kinase targets of each molecule dependent on the cell line were observed. In all cases the largest number of protein kinases per compound were degraded in MOLT-4 cells, followed by KELLY and HEK293T (FIG. 11C). Encouragingly, the target overlap across cell lines for a given compound was good, with ˜50% of the hits in MOLT-4 cells degraded in all 3 cell lines. Cell-line specific kinase hits were also found for 3 of the 4 compounds (FIG. 4D). Whilst a small number of these differences are driven by differences in detection of a particular kinase, a linear relationship between protein expression and protein abundance fold change relative to DMSO (FC) was not globally observed upon degrader treatment across the 3 cell lines (FIG. 4E). This relationship across the dataset was examined by calculating the frequency of degradation for each kinase profiled in MOLT-4 cells. In both cases, a U-shaped relationship was observed between either max FC or degradation frequency and protein expression (FIG. 4D), consistent with that expected from mathematical models of three-body binding equilibria (Douglass Jr et al. J Am Chem Soc 135:6092-6099 (2013)). Together these data indicate it may be more challenging to rapidly degrade kinases with either very high or very low relative expression levels.

There are many examples within the dataset of significantly different degrader efficacy against a given target in the different cell lines with no obvious difference in protein expression; SIK2, LIMK1, FAK (SK-3-91); CSK, MAPK9 (DB0646): GSK3B (WH-10417-099) (FIG. 4C; FIG. 11D). These data indicate that expression is not a regulating factor for degradation of the majority of kinases.

Finally, expression levels of the previously identified poorly-degradable kinases were assessed. DNAPK was identified as the most highly expressed kinase in MOLT-4 cells, potentially explaining its resistance to rapid degradation. CAMKK2 and IKKe have intermediate expression levels, and JAK2 was not quantified in the cell line relative protein expression experiment.

Although target expression did not appear to be the key driver of degradation differences between cell lines, we hypothesized that kinase expression level may alter degradation kinetics. To assess the degradation rate of different protein kinases, MOLT-4 cells were treated with either SK-3-91 or DB0646, at five different time points (1, 2, 4, 8 and 12 h). As a general observation, most kinases showed increased degradation over time in response to each of the degraders, with kinetics ranging from fast (ULK1, PTK2B. LRRK2) to slow (RIOK2, CDK13 or MAP4K5) (FIG. 11E; Appendix I), but no correlation between expression level and degradation rate was observed. Using combined analysis of protein degradation data with relative protein expression analysis no any evidence to support target expression levels as a predictor of propensity for degradation for the majority of kinases was found.

Next, the expression levels of the CRL4CRBN E3 ligase subunits, the E2 enzyme UBE2G1, the p97-unfoldase, proteosome subunits and ABC drug transporters across the 3 cell lines were assessed (FIG. 4E). Expression levels of CUL4A mirror trends were found in the observed numbers of targets per compound in each cell line (MOLT-4>KELLY>HEK293T).

Finally, the relationship between the reported protein half-life and a target's propensity to undergo degradation was examined. Reported kinase half-life data from 3 independent studies and 8 different cell lines or primary cell types were used and compared to the totaled degradation frequency across the dataset described herein (Becher et al., Cell 173:1495-1507.e1418 (2018); Mathieson et al., Nat Commun 9:1-10 (2018); Zechaetal., Mol Cell Proteomics 17(5):974-992 (2018)). Positive correlation was observed between kinase half-lives reported in different studies and different cell types (FIG. 4F; FIG. 11F). A weak negative correlation was observed in the HeLa cell kinase half-life data, where highly degradable kinases had a lower T1/2 (FIG. 4F). No correlation was present between kinase half-life and either degradation frequency or maximal protein abundance fold change in response to degraders in all other cell types (FIG. 4F: FIG. 10F), leading to the conclusion that endogenous protein turnover rate is unrelated to TPD tractability.

The clear differences observed in the potency of molecules for specific targets in distinct cell lines suggest that it is important to examine degrader target profiles and efficiency across different cell lines or tissues in organismal systems. Protein expression levels of CUL4A as correlated with higher numbers of degraded targets for a given molecule across 3 cell lines were identified. The different cellular states, presence of target specific deubiquitinating enzymes, or other factors may also be drivers of such differences.

### Varying the Recruited E3 Ligase can Influence Degrader Selectivity

The propensity of different E3 ligases to ubiquitinate specific proteins has been reported to vary in the literature (Bondeson et al., Cell Chem Biol 25, 78-87.e75 (2018); Smith et al., Nat Commun 10:131 (2019)). These differences have been attributed to the unique protein-protein interactions that may form between the E3 ligase and target. To build upon previous reports (Bondeson et al., Cell Chem Biol 25, 78-87.e75 (2018)), and assess the impact of altering the target scaffold on the ability of the E3 ligase to influence accessible target scope, the degradation profiles of three matched pairs of multitargeted kinase degrader molecules in MOLT-4 cells were compared. Each of the pairs contained the same kinase targeting ligand (either a thienopyrimidine, desmethoxy-TAE684, or GNF-7) and linker, and either a CRBN or a VHL binding moiety, enabling an evaluation of the E3-ligase preference of 86 degraded kinases (FIG. 5A-FIG. 5D).

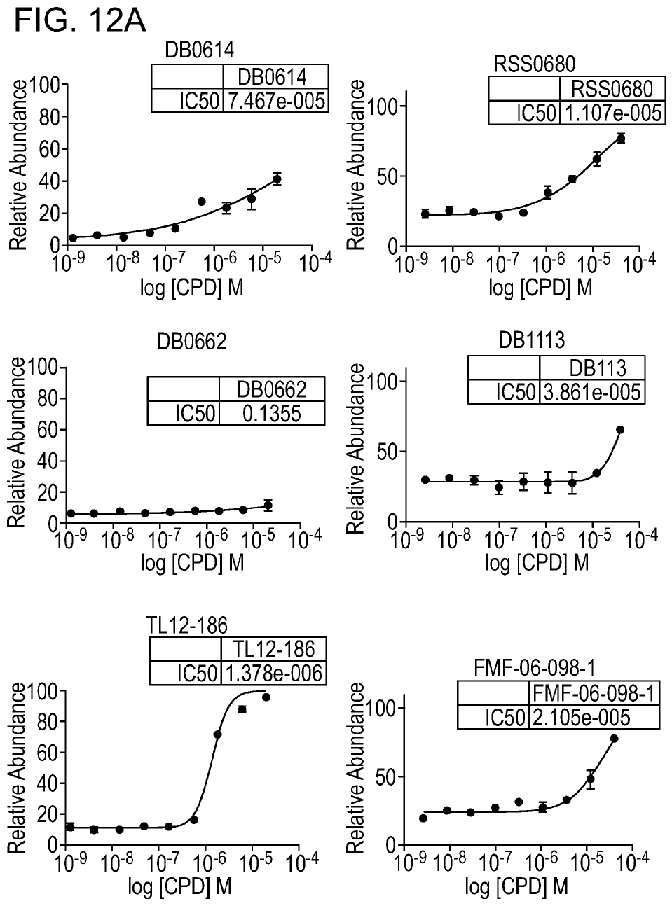

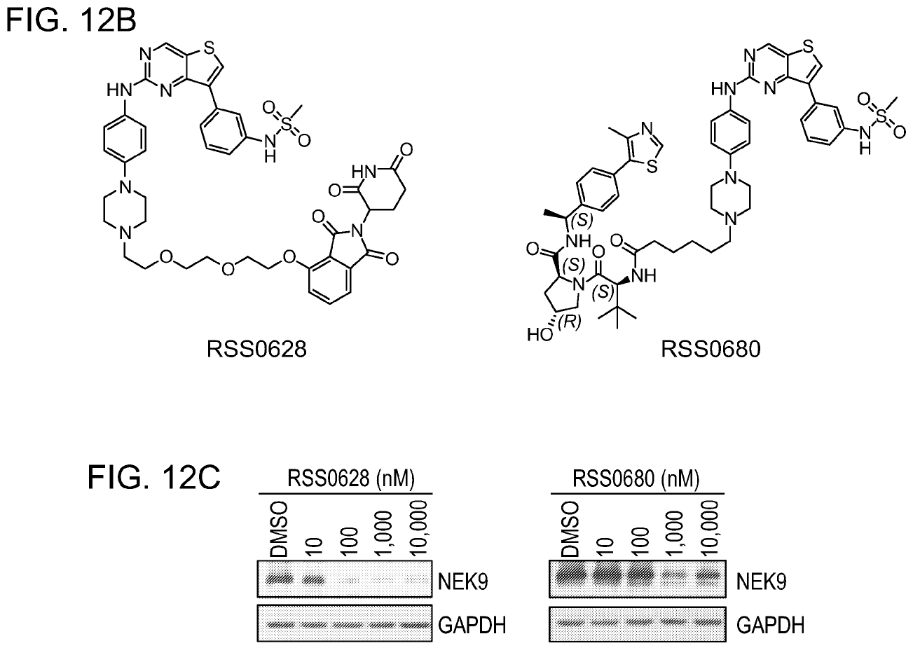

The CRBN and VHL ligands have distinct chemical properties. To rule out differences in cell permeability as a cause for observed differences in target scope, these six degraders were tested in intracellular E3 ligase engagement assays. Side-by-side comparison of each of the matched pairs of degrader molecules revealed only minor differences, with the exception of the desmethoxy-TAE684 based degraders where the CRBN-based degrader was significantly more cell permeable (FIG. 12A).

By altering the ligase recruited, the degradable kinases accessible using these three scaffolds expanded. Seventy unique kinases were degraded by at least one of the three CRBN-recruiting degraders. Upon inclusion of the VHL-recruiting pairs, we identified an additional 16 degraded kinases, corresponding to a 23% increase in kinases targeted. Of the targeted kinases, encouragingly, 50 kinases were degradable by either CRBN or VHL ligase, 16 were exclusive to VHL recruiting compounds and 20 kinases were exclusive to CRBN (FIG. 5D). Whether the nature of the target recruiting ligand impacted the observed ligase preference was assessed. A number of kinases were found to show the same preference across more than one pair. BUB1, LCK, MAP3K11 and SRC were only degradable by CRBN-recruiting degraders, and CDK1, CDK17, MAP4K2 and MAPK7 were only targetable by VHL-recruiting degraders. This ligase preference held true across the whole degradable kinome database for MAP3K11 and SRC, which despite being targeted by many different compounds (8 and 14, respectively), were found to be degraded exclusively by CRBN-recruiting degraders.

In addition to highlighting kinases that are exclusively targeted by one E3 ligase over another, this dataset can be used to assist with compound design and synthetic prioritization by extracting information about which ligase may be more effective at degrading specific targets. Analysis of the thienopyrimidine pair revealed that several kinases, such as CDK4, CDK6 and WEE1 trend evenly between CRBN and VHL, consistent with previous reports, whereas NEK9 is degradable by both ligases but clearly favors CRBN (FIG. 5A, Figure FIG. 11A-FIG. 11F) (Li et al., Cell Chem Biol. 27(1):57-65 (2019); Steinebach et al., Chem Sci 11:3474-3486 (2020)). Evaluation of the GNF-7 pair revealed that the VHL compound degrades 11 of the 14 MAPKs targeted by this pair, and 6 of them are unique to the VHL compound.

However, even within a group of closely related kinases, it was observed that E3 ligase preference may not be conserved, as shown by MAPK9, which was found to be degradable by both ligases but is degraded more effectively by CRBN (FIG. 5B).

Utilizing direct comparisons of CRBN and VHL degrader pairs with three different kinase targeting scaffolds, the magnitude of effect that can be achieved by the addition of a second E3 ligase to the TPD toolbox was quantified. This systematic comparison across scaffolds and ligases provides additional evidence that expanding the number of ligandable E3-ligases may significantly expand the degradable target space, thus justifying efforts towards developing new E3-targeting molecules. In addition, this dataset delivers critical insights into E3 ligase preferences for over 80 protein kinases, which is valuable information for assisting initial designs of new degrader molecules.

Protein Kinases and IMiD Off-Targets have Varied Tolerance for Subtle Changes in Linker Design

Next, how subtle chemical differences in the linker can influence both the activity and the selectivity of degraders was systematically explored. X-ray crystallography studies have demonstrated that the linkers can participate in extensive contacts with both the target and the E3 ligase, leading to structure-based design strategies that focus on optimizing the linker properties, such as chemical composition, length and rigidity (Gadd et al., Nat Chem Biol 13(5):514-521 (2017); Nowak et al., Nat Chem Biol 14:706-714 (2018); Testa el al., Angew Chem Int Ed Engl 132:1744-1751 (2020)). Changes to linker length have proven to significantly alter the selectivity profile of degraders, an example is the pan-BET to BRD4 selective degrader (Nowak et al., Nat Chem Biol 14:706-714 (2018)). Using post hoc PPI docking, it was rationalized that these observed differences in selectivity were likely due to differences in the temary complex conformations available. Similar phenomena have been described for a limited number of kinase examples. For example, varying the linker length of palbociclib-based CRBN recruiting degraders enabled the development of a series of degraders with either selective or dual CDK4/6 degradation profiles (Jiang et al., Angew Chem Int Ed Engl 58:6321-6326 (2019)).

To assess the importance of subtle linker differences more broadly across kinases, we synthesized six multi-kinase targeting degrader molecules designed to cover the linker space and E3 binding exit vector around the previously published multitargeted TL12-186 degrader (Huang et al., Cell Chem Biol 25:88-99.e862018) (FIG. 6A). All six of these compounds were profiled for cellular CRBN engagement to ensure comparable intracellular ligase engagement across the series (FIG. 6B). It was found that of the 26 kinases degraded, a subset showed comparable degradation across all compounds suggesting that they are highly tolerant of linker alterations (FIG. 6C: Appendix I). This result indicates that these specific kinases may have the ability to adopt multiple productive complex conformations with CRBN. This set of kinases is enriched for kinases found to be highly degradable across the dataset—PTK2B, ITK and FER, suggesting that the plasticity of the temary complex may be an important feature of highly degradable targets.

Analysis of the data revealed that the number of kinase targets decreased with increasing linker length—PEG-1 (25), PEG-2 (23) and PEG-3 (18) (FIG. 6C). This is a surprising finding, as PEG-3 is a commonly used linker for degraders and is often trialed early in the degrader optimization process (Brand et al., Cell Chem Biol 26:300-306, e309 (2019); Jiang el al., Angew Chem Int Ed Engl 58:6321-6326 (2019); Smith et al., Nat Commun 10(1):131 (2019)). In multiple use cases, longer linkers have been reported to be more productive at forming temary complexes and inducing degradation than their shorter counterparts (Chan et al., J Med Chem 61:504-513 (2018); Zorba et al., Proc Natl Acad Sci USA 115:E7285-E7292 (2018)). This is in contrast to the data presented here and highlights the difficulty in extrapolating TPD design rules across different E3 ligase-target pairs. In these data, a subset of kinases had strong linker preference, ranging from preference for a specific molecule (CSK, CDK9), preference for short linkers (ABL2. CDK4, CDK5, CDK12 and LIMK2), and specific linker-attachment regioselectivity (CDK7, AAK1, BLK).

Another aspect of target specificity that has shown to be amenable to manipulation of the linker exit vector is the degradation of common IMiD targets that are often a consequence of using IMiD molecules to recruit CRBN. Direct comparison of the expression of known IMiD targets in response to these degraders provided some insights into the linker structure activity relationships for this family of off-targets, revealing that ZNF653 and IKZF1 clearly favor ortho-linked degraders, whereas RNF166 favors meta-linked degraders (FIG. 6D).

Previous studies have shown that a minor nitrogen to oxygen modification on the thalidomide aryl attachment point can significantly reduce or remove IMiD off-target effects in BTK and CDK4/6 targeting degraders (Dobrovolsky et al., Blood 133:952-961 (2019); Jiang et al., Angew Chem Int Ed Engl 58:6321-6326 (2019)). To assess whether this design feature remains applicable over a broad target and scaffold scope, the propensity of the 68 CRBN-recruiting degraders in this database to degrade known IMiD targets was assessed (Donovan et al., Elife 7:e38430 (2018); Sievers et al., Science 362(6414):eaat0572 (2018)). Consistent with the above-mentioned previous reports, it was found that all 34 degraders containing an aryl amine at the thalidomide exit location were able to induce degradation of at least one, but in most cases several, known IMiD targets. Surprisingly, the majority of aryl ether linked degraders also induced degradation of IMiD off-targets, however, 19 of the 25 CRBN-recruiting degraders with an aryl oxy acetamide conjugation to linker showed no degradation of IMiD off-targets, highlighting this as a preferred linker attachment chemistry for selective degraders (FIG. 6E).

Through systematic analysis of linker length and CRBN ligand attachment chemistry on a multi-kinase targeting degrader, it was observed that linker length and ligase orientation have varying effects on the degradation of different protein kinases and common IMiD off-targets such as IKZF1, ZFP91 and RNF166. In the absence of empirical data, or yet-to-be developed predictive models, linker exploration by extensive analog synthesis may be required to find compounds active toward the subset of kinases with narrow linker SAR.

### Proteasomal Degradation of Most Kinases is p97 Dependent

It was hypothesized that the dataset presented herein, in particular the four multi-kinase degraders targeting—100 protein kinases, provides unique tools to study fundamental aspects of ubiquitin biology and induced degradation. The necessity and role of AAA+ ATPase p97 activity upstream of the proteasome is a step in the ubiquitin dependent proteolysis that is poorly understood, p97 unfoldase activity has been demonstrated to be necessary for extracting a subset of proteins marked for degradation from multi-protein complexes, chromatin, or membrane bound complexes (Ramadan et al, Nature 450:1258-1262 (2007): Shcherbik and Haines, Mol Cell 25:385-397 (2007): Verma et al., Mol Cell 41:82-92 (2011)). However, it is unclear what factors determine whether degradation of a ubiquitinated protein occurs in a p97 dependent, or independent manner. The ability to induce rapid polyubiquitination of large numbers of kinases with multitargeted degraders provided an opportunity to examine whether degradation of protein kinases is p97 dependent, and if this dependency changes with differences in recruited E3 ligase or the target.

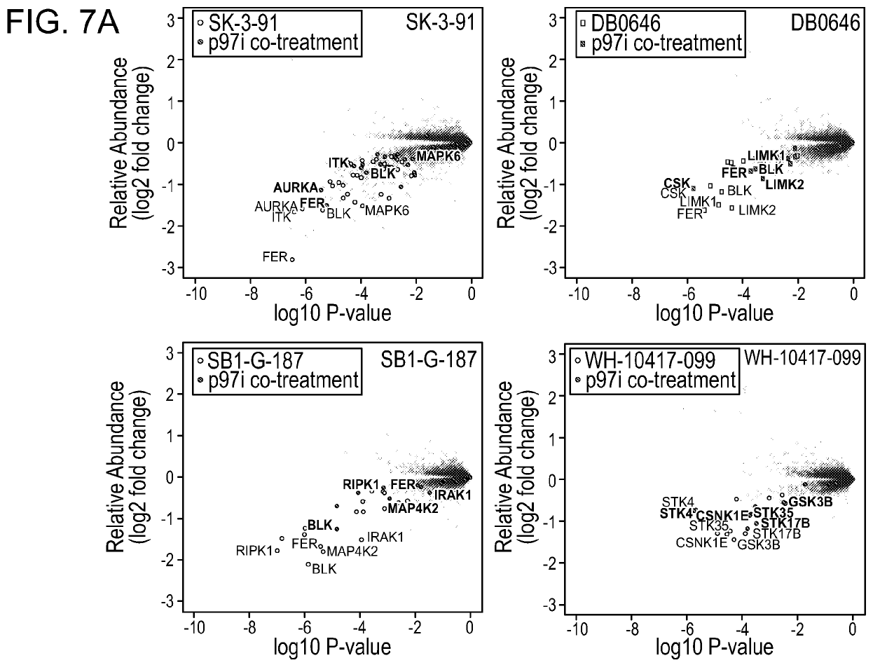

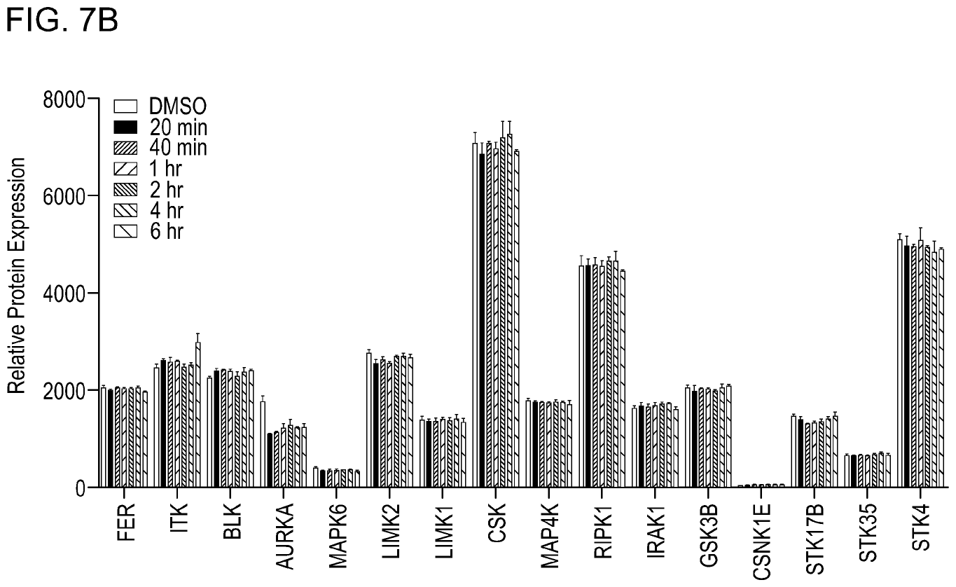

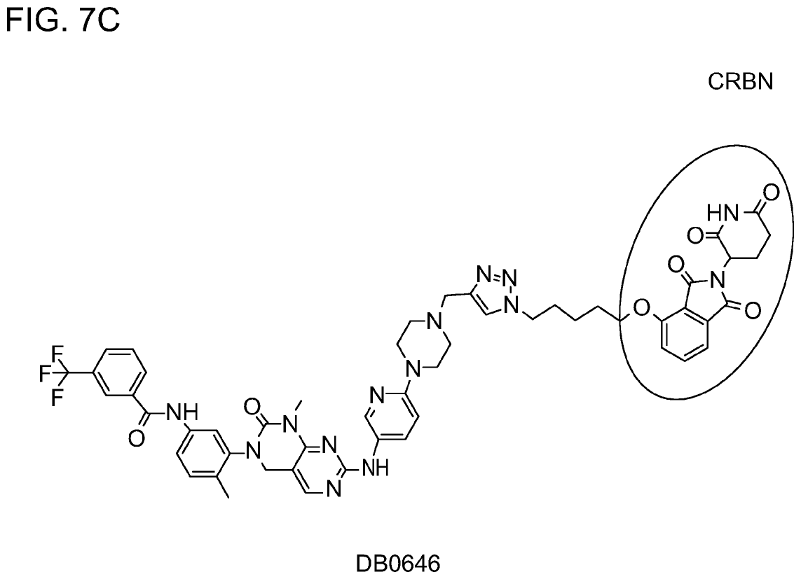

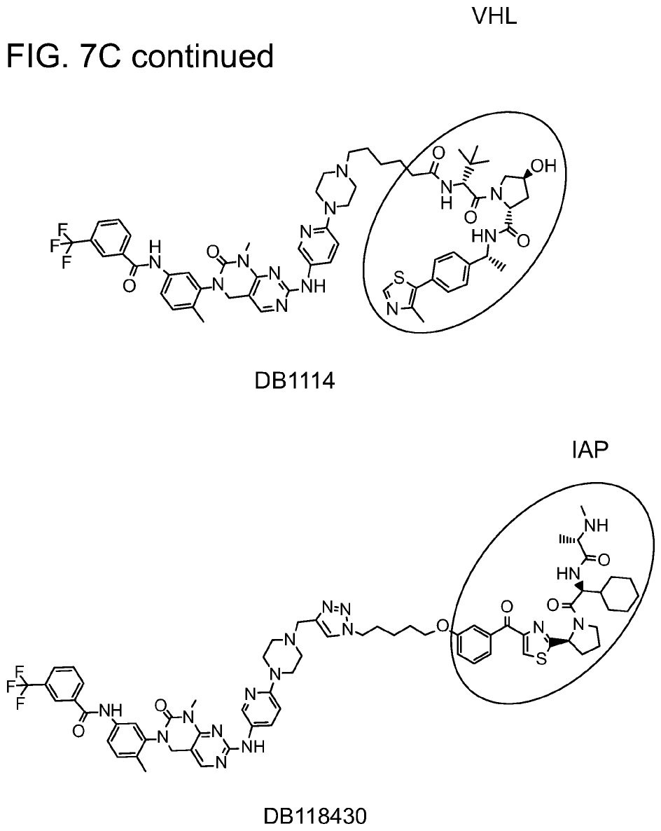

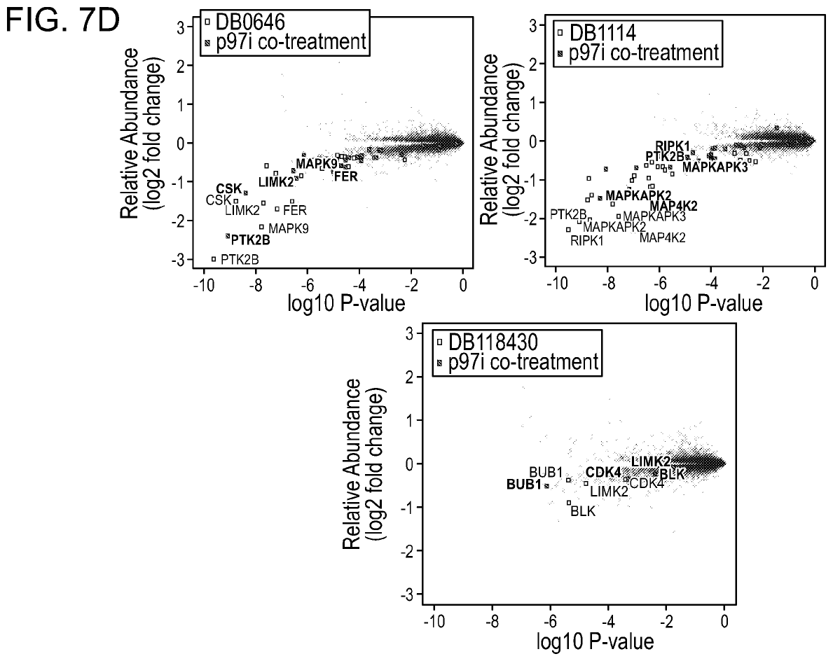

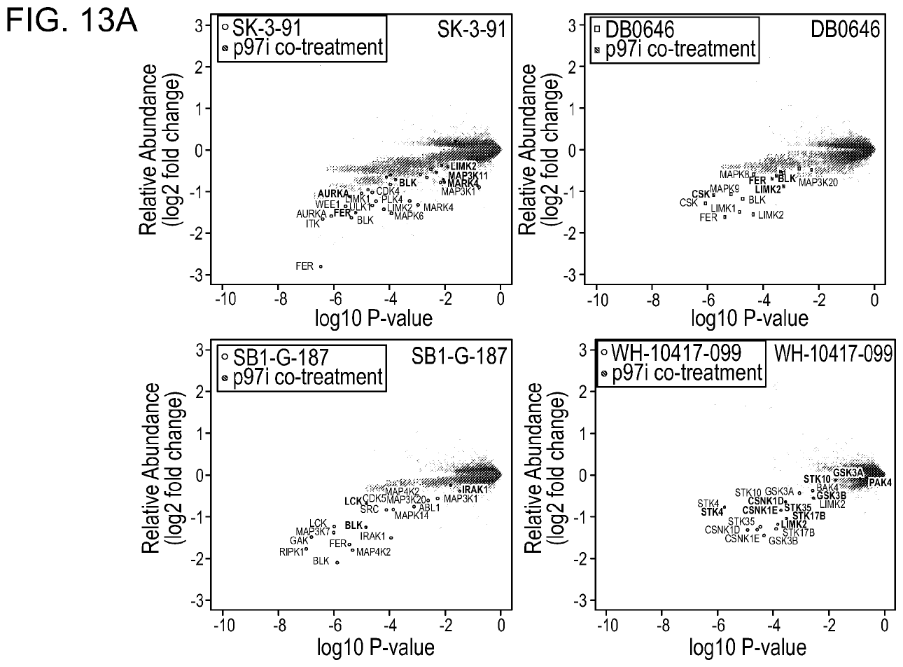

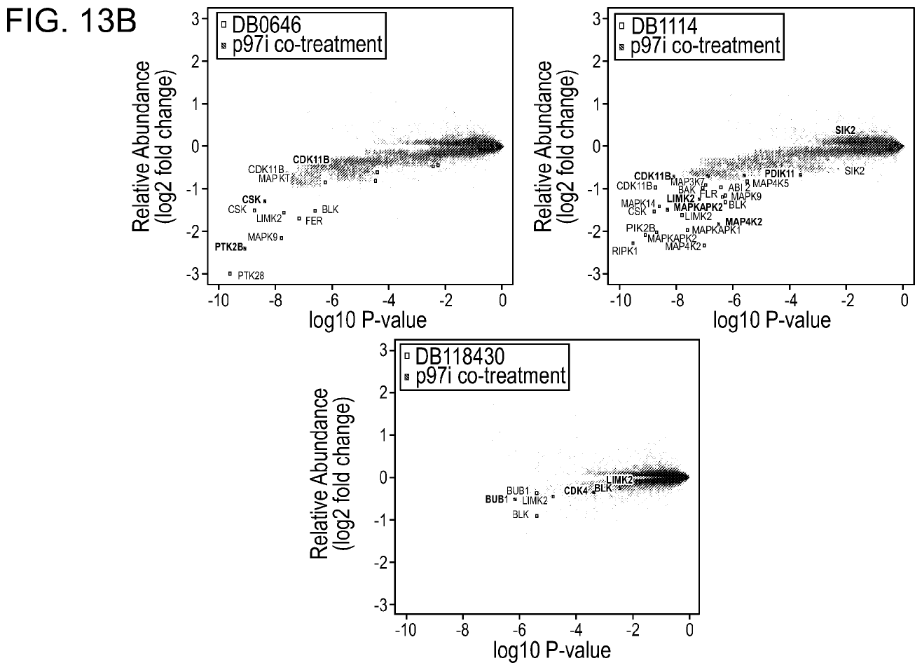

To assess p97 dependence across the kinome, changes in protein abundance in response to treatment with each of our four multi-kinase targeting degraders alone and compared to co-treatment with the p97 inhibitor CB-5083 were measured at the 5-h time point. Analysis of the four treatment groups revealed that almost all of the kinases downregulated in response to degrader treatment show some degradation rescue when p97 is inhibited (FIG. 7A, FIG. 13A). It was observed that CSK and STK4 were exceptions to this trend, as they show very little degradation rescue in the DB0646 and WH-10417-099 rescue treatments, respectively.

Given the major role of p97 in regulating cellular protein degradation, additional experiments were needed to rule out the possibility of indirect effects of p97 inhibition contributing to the p97-dependent degradation rescue observed in response to our kinase degraders. To thoroughly test the broad proteome response to p97 inhibition, global protein expression measurements were performed after a time course (20 min to 6 h) of CB-5083 treatments. Careful analysis of protein expression of the top protein kinase hits from each of the four degraders revealed that expression levels are stable over the 6 h p97 inhibition experiment, confirming that in absence of degrader, p97 inhibition does not cause global blocking of kinase degradation (FIG. 7B).

In addition, it was confirmed that the proposed p97 dependence is independent of the ligase responsible for mediating ubiquitination by comparing results from three multi-kinase targeting degraders based on GNF-7 that recruit different E3 ligases (CRBN, VHL or IAP) (FIG. 7C-FIG. 7D; FIG. 13B).

Taken together, the results suggest that the role of p97 in the handover of substrates to the proteasome goes beyond the extraction of proteins from large cellular structures, but also includes unfolding soluble polyubiquitinated proteins, such as the diverse array of kinases we focus on here, for proteasomal degradation.

This study addressed the current lack of comparable datasets from which to extract general features of TPD-mediated degradation through a wide-ranging analysis of the degradability of the kinome. The use of a curated library of degraders (91) was combined with multiplexed mass spectrometry-based quantitative proteomics to map the degradability of more than 200 kinases across 7 different cell lines. The resulting degradable kinome database represents the first publicly accessible resource of its kind, providing information on the degradability of individual kinases, proteome-wide compound selectivity, and chemical structures of initial lead compounds suitable for further optimization.

Many of the degraders characterized herein represent valuable initial leads for the development of selective degrader chemical probes for understudied kinases—a key goal of the NIH Illuminating the Druggable Genome initiative (Oprea et al., Nat Rev Drug Discov 17:317-332 (2018)). Strikingly, active degrader molecules were found for more than 16 understudied kinases including two potent and selective degraders for CDK17. Assessment of the kinase binding scaffold reveals that the kinase ligand for these two molecules is dabrafenib (Tafinlar®), an approved inhibitor of BRAF V600E mutations in patients with malignant melanoma. Given that dabrafenib is commonly described as a BRAF selective molecule (Rheault et al., ACS Med Chem Lett 4:358-362(2013)), it is extremely unlikely that dabrafenib would feature on the list of initial ligands for beginning a CDK17 selective degrader campaign, yet the selectivity and potency of DD-03-156 is exquisite and would make an advanced starting point for the development of a chemical probe for the degradation of CDK17. This example illustrates how the additional constraints required for degradation can lead to dramatically improved selectivity in the degrader relative to a parental inhibitor, and the significant benefit that informed scaffold selection can have for the identification of starting chemistry and degrader design.

One of the largest challenges in the use of degrader technology is the length of the resource-intensive discovery phase (Burslem and Crews, Cell 181:102-114 (2020)). So far, a number of potential trends or observations to guide rational degrader design have been reported, often only backed by a few exemplified molecules and targets. This first-of-its-kind degradation dataset has sufficient size to allow the assessment of key parameters and evaluation of current hypotheses in the field of degrader design. Herein, it is demonstrated that many factors typically considered important, such a linker length, ligase binding moiety, cellular target occupancy, ternary complex formation or target expression level, play a surprisingly inconsistent role in the efficacy of degraders for kinases, highlighting the need for data-driven approaches. Kinases were successfully sorted according to how they are affected by each of these variables, and this experimentally-determined categorization will prove crucial for the design of optimization workflows and synthetic prioritization. For example, while it can generally be concluded that cellular target engagement is not a good predictor of degrader efficacy, suggesting a catalytic mechanism uncoupled from primary affinities, kinases, wherein an affinity threshold must be met for degradation, such as GCK, were also discovered. Evidence of both productive and unproductive degrader induced temary complex formation with CRBN was found, and it was observed that many kinases were degraded even though they do not form detectable temary complexes, indicating transient or low abundance complexes can result in efficient degradation. While many targets can be degraded with both CRBN and VHL targeting degraders, a significant number show clear preferential compatibility with one over the other. Differences in the target profile of compounds when tested in MOLT-4, KELLY or HEK293T cells were observed, and target expression levels were ruled out as the determining factors driving these differences. Instead, finding that relative protein expression levels of CUL4A, but not CRBN or DDB1, correlated with number of degradation targets of a given molecule in these lines. It was concluded that the downregulated targets of degraders should be characterized in the cellular or in vivo systems in which their effects can be studied. Furthermore, the effects of linker length or connection differences on degradation were found to be highly variable across the kinome. High linker-variant tolerance was observed for the most degradable kinases, indicating that these proteins can form a range of compatible ternary complex conformations with CRBN or VHL. Lastly, structure-activity relationships for IMiD off-target degradation was identified across the dataset, which may facilitate rational design of dual zinc finger:kinase or selective kinase degraders.

Together these conclusions underline the complexity of the degradation-based mechanism of action, and the importance of creating and expanding systematic resources, such as those described herein. Crucially, the database includes negative data, which although often overlooked and underreported is critical for accelerating degrader discovery in the broader community.

Technological advances often facilitate new biological discoveries (Botstein, Mol Biol Cell 21:3791-3792 (2010)). It is demonstrated herein that this database can serve as a rich source of small molecule tools with which to study the basic biology of the ubiquitin proteasome system (UPS), by interrogating the role of the AAA+-ATPase p97. These observations suggest that the majority of the degradable kinome is processed in a p97-dependent fashion, and that this dependence occurs irrespective of the E3 ligase recruited (CRBN, VHL and IAP). Although much still remains to be understood about the role of p97 in facilitating the proteasomal degradation of kinases, this study demonstrates how this collection of multitargeted degraders can be harnessed to reveal effects of perturbations to the UPS on protein degradation across gene families.

This large dataset may accelerate development not only of degrader chemical probes and clinically relevant lead compounds across the kinome, but also of informatics and molecular modeling-based approaches that may lead to improved prediction of degradation activity and rational design of these bifunctional entities.

One of the many conclusions from this work is that starting from the most potent and selective binders of a kinase of interest is not always the best approach for developing a bivalent degrader for that kinase. This is an important finding because this suggests the current dominant approach for making heterobifunctional kinase degraders should be altered, and provides a solution for experimentally identifying the most suitable starting point.

These and other aspects of the present invention will be further appreciated upon consideration of the following Examples, which are intended to illustrate certain particular embodiments of the invention but are not intended to limit its scope, as defined by the claims.

### EXAMPLES

These and other aspects of the present invention will be further appreciated upon consideration of the following Examples, which are intended to illustrate certain particular embodiments of the invention but are not intended to limit its scope, as defined by the claims.

### Data and Code Availability

The proteomics datasets generated during this study are available at PRIDE accession: PXD019142; PXD019143; PXD019144; PXD019242; PXD019168; PXD019167; PXD019166; PXD019164; PXD019165; PXD019171 PXD021255; PXD021313; and PXD021242.

Proteomics data generated during this study are also available at our custom online database (http://dev.dfci-fischerlab.com).

The RNA sequencing data generated during this study is available at GEO accession: GSE157560.

### General Methods

### STAR★Methods

### Cell Culture

HEK293T cells were cultured in DMEM media supplemented with 10% fetal bovine serum. MM1.S, MOLT-4, KELLY, OVCAR-8 and Mino cells were cultured in RPMI-1640 media supplemented with 10% fetal bovine serum. KATO III cells were cultured in IMDM media supplemented with 20% fetal bovine serum. All cells were grown in a 37° C. incubator with 5% CO₂.

TABLE 2
KEY RESOURCES TABLE

| | | |
|---|---|---|
| REAGENT or RESOURCE | SOURCE | IDENTIFIER |

Critical Commercial Assays

| | | |
|---|---|---|
| Tandem Mass Tag (TMT) Reagents | Thermo Fisher | Cat# A90406 |
| | Scientific | Cat# 4808 |
| | | Cat# A44520 |
| Pierce BCA Protein Assay Kit | Life Technologies | Cat# 23225 |
| RNeasy Mini Kit | Qiagen | Cat# 74136 |
| KiNativ | ActivX Biosciences | http://www.kinativ. |
| | | com/ |
| CellTiter-Glo | Promega | Cat# G7570 |

Chemicals, Peptides, and Recombinant Proteins

| | | |
|---|---|---|
| CB-5083 | Med Chem Express | HY-12861 |
| cOmplete, Mini Protease Inhibitor Cocktail | Sigma-Aldrich | Cat# 11836153001 |
| PhosSTOP Phosphatase Inhibitor Tablets | Sigma-Aldrich | Cat# 04906837001 |
| Halt Protease and Phosphatase Inhibitor Single- | Thermo Fisher | Cat# 78442 |
| Use Cocktail | Scientific | |
| dBET6 | Gray Lab | N/A |
| AT1 | Tocris | Cat# 6356 |
| BRD4₈₀₂-GFP-P2a-mCherry | Fischer Lab | N/A |
| | Nowak et al. (2018) | |
| | Nat. Chem. Biol. | |

Deposited Data

| | | |
|---|---|---|
| wp-esf_016 | This paper | PXD019142 |
| wp-esf_103 | This paper | PXD019143 |
| wp-esf_105 | This paper | PXD019144 |
| wp-esf_107 | This paper | PXD019242 |
| wp-esf_117 | This paper | PXD019168 |
| wp-esf_131 | This paper | PXD019167 |
| wp-esf_133 | This paper | PXD019166 |
| wp-esf_152 | This paper | PXD019164 |
| wp-esf_160 | This paper | PXD019165 |
| wp-esf_168 | This paper | PXD019171 |
| wp-esf_172 | This paper | PXD021255 |
| wp_esf_173 | This paper | PXD021313 |
| wp-esf_195 | This paper | PXD021242 |
| RNA sequencing | This paper | GSE157560 |

Experimental Models: Cell Lines

| | | |
|---|---|---|
| HEK293T (H. sapiens) | ATCC | CRL-11268; |
| | | RRID: CVCL_0063 |
| MOLT-4 (H. sapiens) | ATCC | CRL-1582; |
| | | RRID: CVCL_0013 |
| Mino (H. sapiens) | ATCC | CRL-3000; |
| | | RRID.CVCL_1872 |
| MM1.S (H. sapiens) | ATCC | CRL-2974; |
| | | RRID:CVCL_8792 |
| OVCAR-8 (H. sapiens) | Gray Lab | RRID:CVCL_1629 |
| KATO III (H. sapiens) | ATCC | HTB-103: |
| | | RRID:CVCL_0371 |
| KELLY (H. sapiens) | Sigma-Aldrich | 92110411; |
| | | RRID:CVCL_2092 |

Recombinant DNA

| | | |
|---|---|---|
| pKD214 | This paper | pNTM-Flag-CRBN |

Software and Algorithms

| | | |
|---|---|---|
| Proteome Discoverer 2.1, 2.2, 2.4 | Thermo Fisher | RRID: |
| | Scientific | SCR_014477 |
| R Framework | Team RCR: A | http://www.R. |
| | Language | pro.org/ |
| | and Environment for | |
| | Statistical | |
| | Computing | |
| Statistical Analysis | Ritchie et al. (2015) | https://bioconductor. |
| Limma Package | Nucleic Acids Res. | org/packages/rel |
| (R framework) | | ease/bloc/html/limma. |
| | | html |
| GraphPad Prism 8 | | http://www.graphpad. |
| | | com/ |

Antibodies

| | | |
|---|---|---|
| NEK9 (1:1,000) | Santa Cruz Biotech | Cat# SC-100401 |
| GAPDH (1:5,000) | Cell Signaling | Cat# 5174S; |
| | | RRID:AB_10622025 |
| GAPDH (1:5,000) | Santa Cruz Biotech | Cat# sc-47724; |
| | | RRID:AB_627678 |
| AURKA (1:1,000) | Cell Signaling | Cat# D3E4Q; |
| | | RRID:AB_2665504 |
| Goat anti-Mouse IgG-HRP (1:10,000) | genDEPOT | Cat# SA001-500 |
| Goat anti-Rabbit IgG-HRP (1:10,000) | genDEPOT | Cat# SA002-500 |
| Anti-Flag M2 magnetic beads | Millipore Sigma | Cat# M8823; |
| | | RRID:AB_2637089 |

### Example 1: Competitive Displacement Assay for Cellular CRBN and VHL Engagement

HEK293T cells stably expressing the BRD4_{BD2}-GFP with mCherry reporter were seeded at 30-50% confluency in 384-well plates with 50 μL FluoroBrite™ Dulbecco's Modified Eagle's medium (DMEM) media (Thermo Fisher Scientific. A18967) containing 10% fetal bovine serum (FBS) per well a day before compound treatment. Degrader titrations and 100 nM dBET6 or 250 nM AT1 were dispensed using a D300e Digital Dispenser (HP), normalized to 0.5% DMSO, and incubated with cells for 5 hours. Assay plates were imaged using Acumen® (TTP Labtech), as described above. Experiments were performed in triplicates and the values for the concentrations that lead to a 50% increase in BRD4_{BD2}-eGFP accumulation (EC₅₀) were calculated using the nonlinear fit variable slope model (GraphPad Software).

### Example 2: CellTiter-Glo® Viability Assay

MM1.S (purchased from ATCC) was seeded in a 96-well microplate at 10,000 cells per 1130 well in RPMI-1(40 media supplemented with 10% FBS and incubated with compounds (final DMSO concentration at 0.1%). Relative cell viability was measured 72 hours after drug addition using CellTiter-Glo® (Promega) according to the manufacturer's protocol. Each analysis was performed in biological triplicate.

### Example 3: KiNativ® Live Cell Profiling Protocol

CRBN^{-/-} MOLT-4 cells were plated in fresh media (RPMI-1640+10% FBS) in 15 cm plates and treated for 5 hours with candidate compounds. To harvest cells, plates were harvested using detachment using CellStripper™ detachment solution (Corning®) and washed 2× with cold phosphate-buffered saline (abbreviated PBS), followed by centrifugation and snap-freezing of cell pellets in liquid nitrogen. The remainder of the KiNativ® profiling experiment was performed by ActivX® Biosciences (La Jolla. CA).

### Example 4: RNA Sequencing

MOLT-4 cells were seeded into 24 T25 flasks with 10 mL of culture at 10⁶ cells/mL prior to compound treatment. Cells were treated in four replicates each with either 0.05% dimethyl sulfoxide (DMSO) or 1 μM SK-3-91 for a total duration of 1, 2, 4 or 8 hours. Cells were harvested using CellStripper™ Dissociation reagent (Corning®), washed twice with PBS, and followed by snap freezing in liquid nitrogen. Total RNA was isolated from cell pellets using the RNeasy® Mini Kit (Qiagen®) following the manufacturer's directions. For quality control, RNA concentration and rRNA ratio (28S/18S) were measured using an Agilent 2100 Bioanalyzer. Samples were submitted to BGI Group for RNA-seq library preparation and Next Generation Sequencing using the BGISEQ-500 platform producing 50 base-pair single-end reads. Sequencing reads were aligned to the human genome (BSgenome.Hsapiens.UCSC.hg19 Bioconductor package, using splicedAlignment=FALSE) and quantified at the level of genes (TxDb.Hsapiens.UCSC.hg19.knownGene Bioconductor package) using the QuasR package with default parameters (Gaidatzis et al., Bioinformatics 31(7):1130-1132 (2015)). Expressed genes were identified using the edgeR Bioconductor package (Robinson et al., Bioinformatics 26(1):139-140 (2010)).

### Example 5: Immunoblots

Cells were treated with indicated compounds and doses for 4 hours and washed with ice-cold PBS once. Cells were lysed in an NP40 buffer (50 mM Tris-HCl pH 7.5, 1% NP40, 1 mM ethylenediaminetetraacetic acid (EDTA), 150 mM NaCl, 5 mM NaVO₄ and 2.5 mM NaF) containing a protease inhibitor cocktail (Roche®, 11873580001) or a Triton™ buffer (20 mM Tris HCl pH 7.5, 150 mM NaCl, 1 mM EDTA, 1 mM egtazic acid (EGTA), 1% Triton®, 2.5 mM sodium pyrophosphate, 1 mM P-glycerophosphate, 1 mM Na₃VO₄, 1 μg/ml leupeptin) containing halt protease and phosphatase inhibitor cocktail (Thermo Fisher Scientific, 1166 78442). Protein quantification was performed using Pierce™ BCA Protein Assay (Life Technologies™). Equal amounts of each lysate were loaded and separated on an 8% SDS-PAGE gel and transferred to polyvinylidene difluoride (PVDF) membrane. All primary antibodies were diluted in Tris-buffered saline (TBS) containing 0.05% Tween®-20 were incubated overnight. After three washes with Tris-buffered saline 0.1%—Tween®-20 (TBS-T), secondary antibodies were incubated for 1 hour. EnhancedChemiLuminescence solution (ECL) (Lugen LGW-P1001, Korea) was dropped on the membrane and exposed to X-ray film (Agfa. Japan).

### Example 6: Affinity Purification Tandem Mass Tag (TMT) LC-MS3 Mass Spectrometry

HEK293T cells were seeded into 15 cm plates and cells were transiently transfected with 8 μg of pNTM-FLAG-CRBN construct using lipofectamine 2000. 30 hours post transfection, cells were co-treated for 5 hours with 0.1 μM bortezomib and 1 μM of either SK-3-91, DB0646, SB1-G-187, WH-0417099 in biological triplicates or pomalidomide or DMSO control in biological duplicates. Cells were harvested with non-enzymatic CellStripper™ Dissociation reagent (Corning®), followed by three washes with cold PBS and snap freezing. Cell lysis was performed by the addition of IP lysis buffer (50 mM Tris. pH 7.5, 0.5% NP-40, 1 mM EDTA, 10% glycerol and 200 mM NaCl) containing protease inhibitor cocktail (cOmplete™) and relevant co-treatment (above), followed by end-over-end rotation at 4° C. for 3 hours. Lysate was clarified by centrifugation and salt concentration diluted to 100 mM NaCl with the addition of 0 mM NaCl lysis buffer (containing protease inhibitors and 1 μM of relevant compounds to retain temary complexes throughout binding). Lysate was added to 20 μL of pre-washed anti-FLAG M2 magnetic bead slurry (MilliporeSigma) and incubated with end-over-end rotation at 4° C. overnight. Beads were washed six times with 100 mM NaCl lysis buffer containing 1 μM of relevant degraders to retain ternary complexes throughout wash steps.

Proteins were eluted in a two-step elution with the addition of 0.1 M Glycine hydrochloride (MilliporeSigma) and elution buffered to pH 8.5 using 200 mM Tris buffer, pH 8.5. Protein eluates were reduced, alkylated and precipitated using methanol/chloroform as previously described in Donovan et al., eLife 7:e38430 (2018), and the resulting washed precipitated protein was allowed to air dry. Protein pellets were resuspended in 50 μL of EPPs pH 8 and first digested with 2 μg LysC for 12 h at room temperature (RT), followed by 1 μg of trypsin for 6 hours at 37° C. Tandem mass tag (TMT) reagents (Thermo Fisher Scientific) were dissolved in anhydrous acetonitrile (ACN) according to manufacturer's instructions. Anhydrous ACN was added to each peptide sample to a final concentration of 30% v/v, and labeling was induced with the addition of 4 μL of TMT reagent to each sample. The 16-plex labeling reactions were performed for 1 hour at RT and the reaction quenched by the addition of hydroxylamine to a final concentration of 0.3% for 15 minutes at RT. Each of the sample channels were combined in a 1:1 ratio, desalted using C18 solid phase extraction plates (SOLA™, Thermo Fisher Scientific) and analyzed by LC-MS.

### Example 7: Sample Preparation TMT LC-MS3 Mass Spectrometry

Cells were treated with DMSO (biological triplicate) or degrader at indicated dose and time and cells were harvested by centrifugation. Lysis buffer (8 M Urea, 50 mM NaCl, 50 mM 4-(2hydroxyethyl)-1-piperazineethanesulfonic acid (EPPS) pH 8.5, protease and phosphatase inhibitors) was added to the cell pellets and homogenized by 20 passes through a 21-gauge (1.25 in. long) needle to achieve a cell lysate with a protein concentration between 1-4 mg mL-1. A bradford (Bio-Rad) was used to determine the final protein concentration in the cell lysate, 100-200 μg of protein for each sample was reduced, alkylated and precipitated using methanol/chloroform as previously described in Donovan et al., Elife 7:e38430 (2018), and the resulting washed precipitated protein was allowed to air dry. Precipitated protein was resuspended in 4 M Urea, 50 mM HEPES pH 7.4, followed by dilution to 1 M urea with the addition of 200 mM EPPS, pH 8. Proteins were first digested with LysC (1:50: enzyme:protein) for 12 hours at room temperature. The LysC digestion was diluted to 0.5 M Urea with 200 mM EPPS pH 8 followed by digestion with trypsin (1:50; enzyme:protein) for 6 hours at 37° C. Tandem mass tag (TMT) reagents (Thermo Fisher Scientific) were dissolved in anhydrous acetonitrile (ACN) according to manufacturer's instructions. Anhydrous ACN was added to each peptide sample to a final concentration of 30% v/v, and labeling was induced with the addition of TMT reagent to each sample at a ratio of 1:4 peptide:TMT label. The 10, 11, or 16-plex labeling reactions were performed for 1.5 hours at room temperature and the reaction quenched by the addition of hydroxylamine to a final concentration of 0.3% for 15 minutes at room temperature. The sample channels were combined at a 1:1 ratio, desalted using C18 solid phase extraction cartridges (Waters®) and analyzed by LC-MS for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak®, Waters®). Samples were then offline fractionated into 96 fractions by high pH reverse-phase HPLC (Agilent® LC1260) through an aeris peptide xb-c18 column (Phenomenex®) with mobile phase A containing 5% acetonitrile and 10 mM NH₄HCO₃ in LC-MS grade H₂O, and mobile phase B containing 90% acetonitrile and 10 mM NH₄HCO₃ in LC-MS grade H₂O (both pH 8.0). The 96 resulting fractions were then pooled in a non-continuous manner into 24 fractions and desalted using C18 solid phase extraction plates (SOLA®, Thermo Fisher Scientific) followed by subsequent mass spectrometry analysis.

Data were collected using an Orbitrap Fusion™ Lumos™ mass spectrometer (Thermo Fisher Scientific, San Jose, CA, USA) coupled with a Proxeon EASY-nLC™ 1200 LC pump (Thermo Fisher Scientific) or an Orbitrap Eclipse™ Tribrid™ mass spectrometer (Thermo Fisher Scientific, San Jose, CA, USA) coupled with an UltiMate™ 3000 RSLCnano System. Peptides were separated on an EasySpray™ ES803a/ES803a.rev2 75 μm inner diameter microcapillary column (ThermoFisher Scientific). Peptides were separated using a 190 min gradient of 6-27% acetonitrile in 1.0% formic acid with a flow rate of 350 nL/min.

Each analysis used an MS3-based TMT method as described previously(McAlister et al., 2014). The data were acquired using a mass range of m/z 340-1350, resolution 120,000, automatic gain control (AGC) target 5×10⁵, maximum injection time 100 ms, dynamic exclusion of 120 seconds for the peptide measurements in the Orbitrap. Data dependent MS2 spectra were acquired in the ion trap with a normalized collision energy (NCE) set at 35%, AGC target set to 1.8×10⁴ and a maximum injection time of 120 ms. MS3 scans were acquired in the Orbitrap with HCD collision energy set to 55%. AGC target set to 2×10⁵, maximum injection time of 150 ms, resolution at 50,000 and with a maximum synchronous precursor selection (SPS) precursors set to 10.

### Example 8: LC-MS Data Analysis

Proteome Discoverer™ 2.1, 2.2 or 2.4 (Thermo Fisher Scientific) was used for .RAW file processing and controlling peptide and protein level false discovery rates, assembling proteins from peptides, and protein quantification from peptides. MS/MS spectra were searched against a Uniprot human database (September 2016 or December 2019) with both the forward and reverse_sequences. Database search criteria are as follows: tryptic with two missed cleavages, a precursor mass tolerance of 20 ppm, fragment ion mass tolerance of 0.6 Da, static alkylation of cysteine (57.02146 Da), static TMT labelling of lysine residues and N-termini of peptides (229.16293 Da), and variable oxidation of methionine (15.99491 Da). TMT reporter ion intensities were measured using a 0.003 Da window around the theoretical m/z for each reporter ion in the MS3 scan. Peptide spectral matches with poor quality MS3 spectra were excluded from quantitation (summed signal-to-noise across channels <100 (Whole Proteome) or <50 (Affinity Purification) and precursor isolation specificity <0.5), and resulting data was filtered to only include proteins that had a minimum of 2 unique peptides quantified. Reporter ion intensities were 1264 normalized and scaled using in-house scripts in the R framework (R Development Core Team, 2014). Statistical analysis was carried out using the limma package within the R framework (Ritchie et al., Nucleic Acids Res. 43:e47 (2015)).

### Example 9: General Chemistry Methods

Unless otherwise noted, reagents and solvents were obtained from commercial suppliers and were used without further purification. 1H NMR spectra were recorded on 500 MHz Bruker Avance™ III spectrometer, and chemical shifts are reported in parts per million (ppm, δ) downfield from tetramethylsilane (TMS). Coupling constants (J) are reported in Hz. Spin multiplicities are described as s (singlet), br (broad singlet), d (doublet), t (triplet), q (quartet), and m (multiplet). Mass spectra were obtained on a Waters® Acquity UPLC. Preparative HPLC was performed on a 1276 Waters® Sunfire™ C18 column (19 mm×50 mm, 5 μM) using a gradient of 15-95% methanol in water containing 0.05% trifluoroacetic acid (TFA) over 22 minutes (28 minutes run time) at a flow 1278 rate of 20 mL/min. Assayed compounds were isolated and tested as TFA salts. Purities of assayed 1279 compounds were in all cases greater than 95%, as determined by reverse-phase HPLC analysis.

### ABBREVIATIONS

AIBN: azobisisobutyronitrile
DIEA: diisopropylethylamine
HATU: hexafluorophosphate azabenzotriazole tetramethyl uronium
DCM: dichloromethane
DCE: dichloroethane
DMF: dimethylformamide
m-CPBA: meta-chloroperoxybenzoic acid
NBS: N-bromosuccinimide
PE: petroleum ether
RT: room temperature

### Example 10: Synthesis of Synthesis of (2S,4R)-1-((S)-2-(3-(2-(2-(4-(4-((5-chloro-4-((2-(isopropylsulfonyl)phenyl)amino)pyrimidin-1320 2-yl)amino)phenyl)piperazin-I-yl)ethoxy)ethoxy)propanamido)-3,3-dimethylbutanoyl)-4-1321 hydroxy-N—((S)-1-(4-(4-methylthiazol-5-yl)phenyl)ethyl)pyrrolidine-2-carboxamide (FMF-06-098-1)

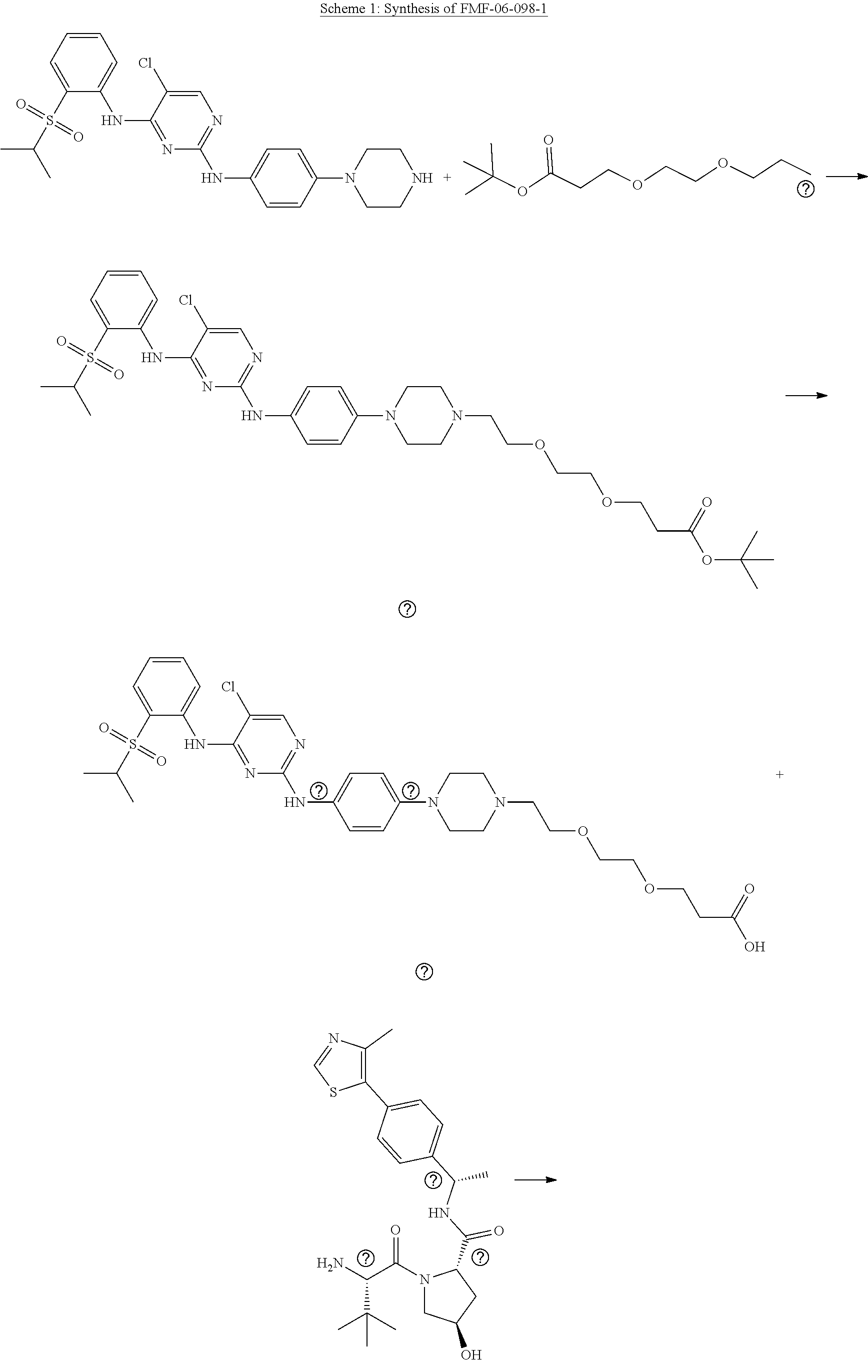

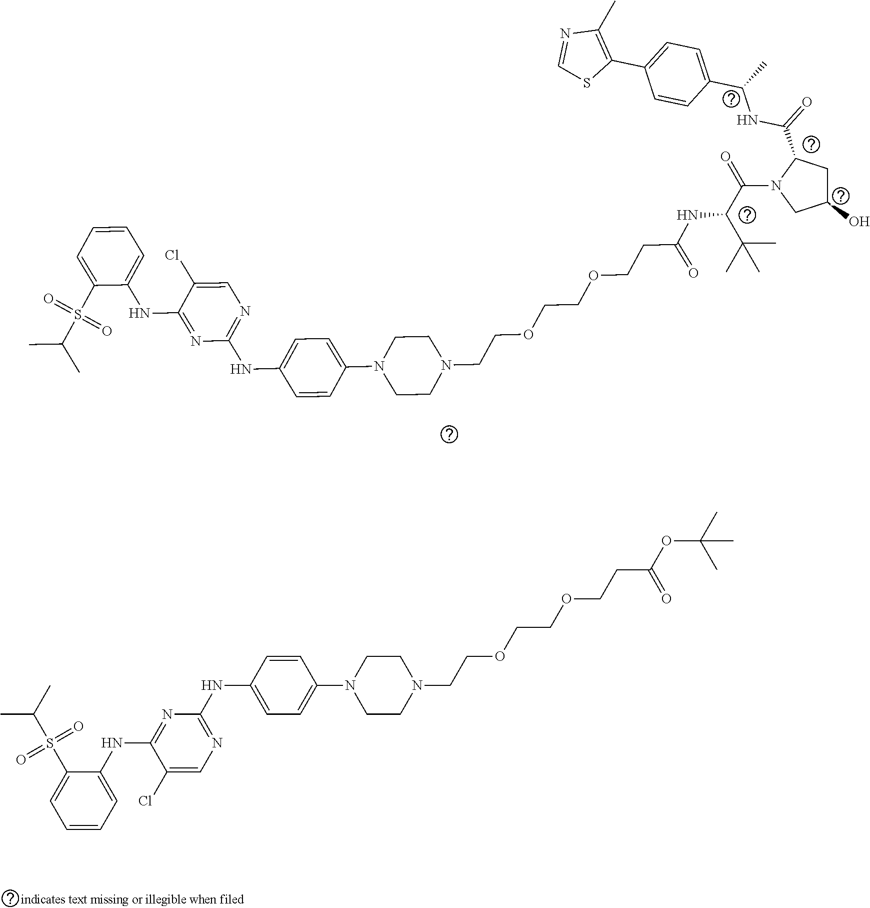 [SMILES: Cc1ncsc1-c1ccc([C@H](C)NC(=O)[C@@H]2C[C@@H](O)CN2C(=O)[C@@H](NC(=O)CCOCCOCCN2CCN(c3ccc(Nc4ncc(Cl)c(Nc5ccccc5S(=O)(=O)C(C)C)n4)cc3)CC2)C(C)(C)C)cc1 ; CC(C)S(=O)(=O)c1ccccc1Nc1nc(Nc2ccc(N3CCN(CCOCCOCCC(=O)OC(C)(C)C)CC3)cc2)ncc1Cl]

### tert-Butyl 3-(2-(2-(4-(4-((5-chloro-4-((2-(isopropylsulfonyl)phenyl)amino) pyrimidin-2-yl)amino)phenyl)piperazin-1-yl)ethoxy)ethoxy)propanoate (FMF-06-091-1)

Intermediate 1 was prepared according to the literature (Huang et al., Cell Chem Biol 25: 88-99 (2018)). Intermediate 1 (25 mg, 0.048 mmol), tert-butyl 3-(2-(2-bromoethoxy)ethoxy)propanoate (16 mg, 0.058 mmol) and potassium carbonate (20 mg, 0.144 mmol) were stirred in MeCN (3 mL) at 80° C., and monitored by UPLC/MS. The reaction mixture was cooled to RT, diluted with water (5 mL) and extracted with DCM (3×10 mL). The pooled organic layers were dried over MgSO₄, filtered and concentrated under reduced pressure. The residue was purified by column chromatography (10:1, DCM:MeOH) to yield 31 mg of FMF-06-091-1 as a colorless oil.

¹H NMR (500 MHz, MeOD) δ 8.53 (d, J=8.5 Hz, 1H), 7.99 (s, 1H), 7.77 (dd, J=7.9, 1.7 Hz, 1H), 7.53 (ddd, J=8.7, 7.3, 1.6 Hz, 1H), 7.33-7.26 (m, 2H), 7.24-7.17 (m, 1H), 6.87-6.80 (m, 2H), 3.64-3.56 (m, 4H), 3.51 (s, 4H), 3.07 (t, J=5.1 Hz, 4H), 2.64 (t, J=5.0 Hz, 4H), 2.57 (dd, J=11.3, 5.7 Hz, 3H), 2.38 (t, J=6.2 Hz, 2H), 1.35 (s, 9H), 1.15 (d, J=6.8 Hz, 6H).

LC/MS (EST) m/z 704.2 [[M+H]⁺ calcd for C₃₄H₄₇ClN₆O₆S: 703.301.

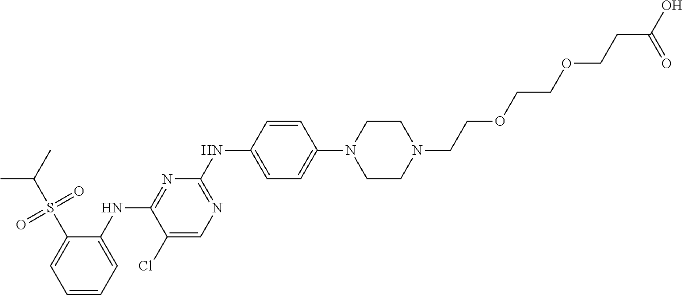 [SMILES: CC(C)S(=O)(=O)c1ccccc1Nc1nc(Nc2ccc(N3CCN(CCOCCOCCC(=O)O)CC3)cc2)ncc1Cl]

### 3-(2-(2-(4-(4-((5-chloro-4-((2-(isopropylsulfonyl)phenyl)amino)pyrimidin-2yl)amino)phenyl)piperazin-1-yl)ethoxy)ethoxy)propanoic acid (FMF-06-095-1)

FMF-06-091-1 (31 mg, 0.044 mmol) was dissolved in 10 mL of DCM, to which was added 1 mL TFA. The reaction was stirred at RT for 2 hours, concentrated under reduced pressure, and used in the next step without further purification (FMF-06-095-1, 30 mg, 0.039 mmol).

LC/MS (ESI) m/z 648.3 [[M+H]⁺; calcd for C₃₀H₃₉ClN₆O₆S: 647.19].

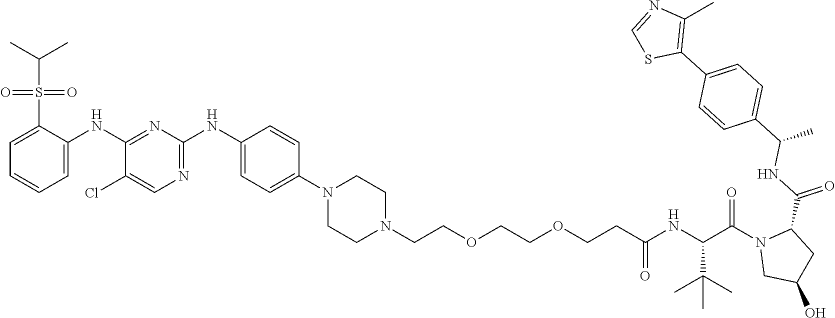 [SMILES: Cc1ncsc1-c1ccc([C@H](C)NC(=O)[C@@H]2C[C@@H](O)CN2C(=O)[C@@H](NC(=O)CCOCCOCCN2CCN(c3ccc(Nc4ncc(Cl)c(Nc5ccccc5S(=O)(=O)C(C)C)n4)cc3)CC2)C(C)(C)C)cc1]

### FMF-06-098-1

FMF-06095-1 (30 mg, 0.039 mmol), VHL ligand (Raina et al., Proc. Natl. Acad. Sci. U.S.A. 113:7124-7129 (2016)) (23 mg, 0.51 mmol), HATU (21 mg, 0.055 mmol), DIPEA (26 μL, 0.140 mmol) were dissolved in DMF and stirred for 16 hours. The reaction mixture was filtered and purified by preparative phase HPLC to give FMF-06-098-1 as a colorless oil (46 mg, trifluoroacetate salt, 0.038 mmol).

¹H NMR (500 MHz, DMSO) δ 9.85 (s, 2H), 9.51 (s, 1H), 9.40 (s, 1H), 8.98 (d, J=4.3 Hz, 1H), 8.65 (s, 1H), 8.37 (d, J=7.8 Hz, 1H), 8.25 (d, J=1.3 Hz, 1H), 7.91-7.83 (m, 2H), 7.77-7.72 (m, 1H), 7.50 (d, J=8.7 Hz, 3H), 7.43 (dt, J=7.3, 2.2 Hz, 2H), 7.38 (dt, J=8.3, 3.5 Hz, 4H), 6.95 (dd, J=8.9, 3.7 Hz, 3H), 4.92 (q, J=7.3 Hz, 1H), 4.55 (d, J=9.4 Hz, 1H), 4.43 (t, J=8.1 Hz, 1H), 4.29 (s, 1H), 3.80 (t, J=4.9 Hz, 2H), 3.56 (q, J=5.1, 4.5 Hz, 1H), 3.75 (d, J=12.5 Hz, 1H), 3.45 (p, J=6.8 Hz, 1H), 3.39 (s, 3H), 3.22 (s, 1H), 3.02 (d, J=12.3 Hz, 2H), 2.59-2.53 (m, 1H), 2.46 (d, J=3.0 Hz, 4H), 2.40 (dt, J=14.7, 6.1 Hz, 1H), 2.07-1.98 (m, 1H), 1.80 (ddd, J=12.9, 8.6, 4.6 Hz, 1H), 1.37 (d, J=6.9 Hz, 1H), 1.17 (d, J=6.8 Hz, 1H), 0.95 (s, 7H).

¹³C NMR (126 MHz, DMSO) δ 171.04, 170.62, 169.89, 162.99, 158.25, 151.97, 129.29, 126.85, 121.61, 116.88, 70.05, 69.67, 69.01, 67.35, 64.75, 59.04, 56.81, 55.35, 54.01, 51.76, 48.17, 46.62, 42.27, 40.50, 40.43, 40.33, 40.28, 40.17, 40.00, 39.83, 39.67, 39.50, 35.87, 26.88, 22.86, 18.53, 17.20, 16.45, 15.33, 12.88.

LC/MS (ESI) m/z 1074.9 [[M+H]⁺; calcd for C₅₃H₉ClN₁₀O₈S₂: 1073.771.

### Example 11: Synthesis of N-(2-(4-((6-(2,6-dichlorophenyl)-8-methyl-7-oxo-7,8-dihydropyrido[2,3-d]pyrimidin-2-yl)amino)phenoxy)ethyl)-7-(2-((2-(2,6-dioxopiperidin-3-yl)-1,3-dioxoisoindolin-4-1413 yl)oxy)acetamido)heptanamide (SB1-G-192)

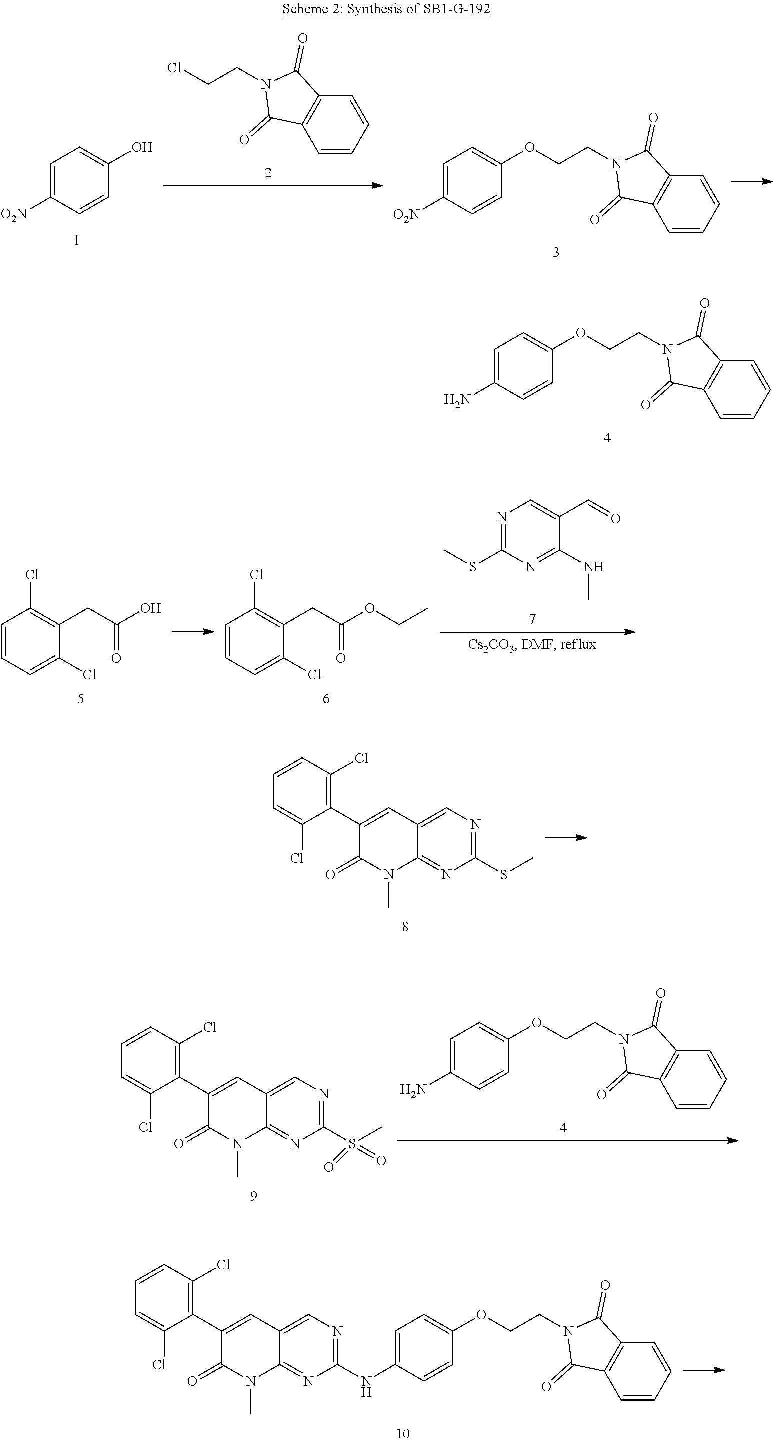

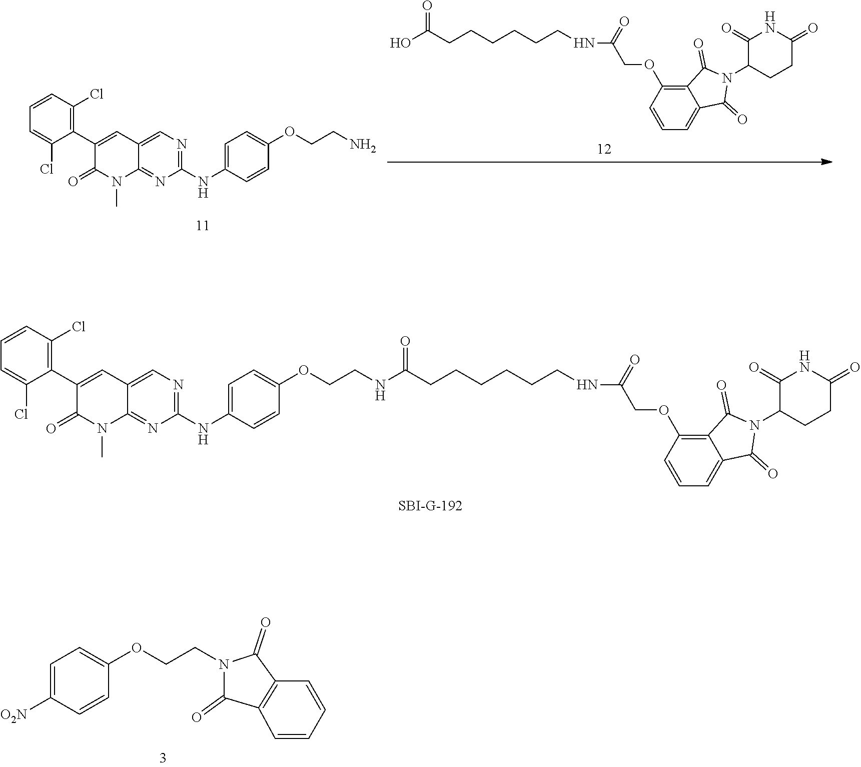 [SMILES: Cn1c(=O)c(-c2c(Cl)cccc2Cl)cc2cnc(Nc3ccc(OCCN)cc3)nc21 ; O=C(O)CCCCCCNC(=O)COc1cccc2c1C(=O)N(C1CCC(=O)NC1=O)C2=O ; Cn1c(=O)c(-c2c(Cl)cccc2Cl)cc2cnc(Nc3ccc(OCCNC(=O)CCCCCCNC(=O)COc4cccc5c4C(=O)N(C4CCC(=O)NC4=O)C5=O)cc3)nc21 ; O=C1c2ccccc2C(=O)N1CCOc1ccc([N+](=O)[O-])cc1]

### 2-(2-(4-Nitrophenoxy)ethyl)isoindoline-1,3-dione (3)

The mixture of 4-nitrophenol (5.564 g, 40.0 mmol), 2-(2-chloroethyl)isoindoline-1,3-dione (9.041 g, 1348 43.1 mmol) and Cs₂CO—; (23.4 g, 71.8 mmol) in DMF (80 mL) was stirred at 100° C. for 16 hours. The mixture was diluted with water(500 mL) and extracted with DCM (300 mL×2), the combined organic layers were dried over anhydrous Na₂SO₄, filtered and concentrated under reduced pressure. The residue was purified by recrystallization from EtOH to give compound 3 as an off-white solid (7.8 g, yield 71%).

LC/MS (EST) m/z: 313.1 [M+H]⁺.

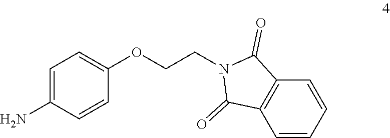 [SMILES: Nc1ccc(OCCN2C(=O)c3ccccc3C2=O)cc1]

### 2-(2-(4-Aminophenoxy)ethyl)isoindoline-1,3-dione (4)

The mixture of compound 3 (6.6 g, 21.1 mmol), Fe (5.9 g, 105.6 mmol), NH₄Cl (6.7 g, 126.8 mmol) and concentrated HCl solution (10.6 mL, 12 M) in EtOH (200 mL) was stirred at 90° C. for 2 hours. The mixture was filtered through Celite®. The filtrate was concentrated under reduced pressure and purified by column chromatography (silica gel, DCM/MeOH=20/1-5/1) to get compound 4 as an off-white solid (5.0 g, yield 84%).

LC/MS (ESI) m/z: 283.1 [M+H]⁺.

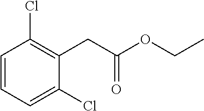

### Ethyl 2-(2,6-dichlorophenyl) acetate (6)

A mixture of compound 5 (25 g, 121.9 mmol) and concentrated H₂SO₄ (15 mL) in EtOH (200 mL) was heated to reflux for 8 hours. The mixture was concentrated to remove most of the organic solvent. The residue was diluted with water (500 mL). The pH was adjusted to 8-9 with aqueous Na₂CO₃ before extraction with DCM (300 mL×2). The combined pooled organic layers were dried over anhydrous Na₂SO₄, filtered, and concentrated under reduced pressure. The residue was purified by column chromatography (silica gel, (PE/EtOAc=20/1-4/1) to give compound 6 as an off-white solid (23.5 g, yield 83%).

LC/MS (ESI) m/z: 232.9 [M+H]⁺.

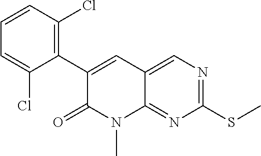 [SMILES: CSc1ncc2cc(-c3c(Cl)cccc3Cl)c(=O)n(C)c2n1]

### 6-(2,6-Dichlorophenyl)-8-methyl-2-(methylthio)pyrido[2.3-d]pyrimidin-7(8H)-one (8)

A mixture of compound 6 (5.0 g, 21.4 mmol), compound 7 (2.63 g, 14.3 mmol) and K₂CO₃ (11.8 1375 g, 85.8 mmol) in DMSO (150 mL) was stirred at 100° C. for 16 hours. The mixture was concentrated to remove most of the organic solvent. The residue was diluted with water (200 mL) and filtered. The resulting cake was washed with EtOAc/PE=1/3 (50 mL) to give compound 8 as an off-white solid (1.85 g, yield 24%).

LC/MS (EST) m/z: 351.9 [M+H]⁺.

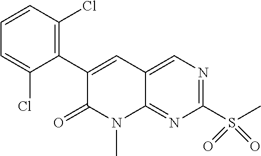 [SMILES: Cn1c(=O)c(-c2c(Cl)cccc2Cl)cc2cnc(S(C)(=O)=O)nc21]

### 6-(2,6-Dichlorophenyl)-8-methyl-2-(methylsulfonyl)pyrido[2,3-d]pyrimidin-7(8H)-one (9)

A mixture of compound 9 (1.6 g, 4.5 mmol) and m-CPBA (1.9 g, 11.3 mmol) in MeOH (50 mL) was stirred at 25° C. for 16 hours. The mixture was concentrated to remove most of the organic solvent. The residue was diluted with water (100 mL) and extracted with DCM (200 mL×2). The combined organic layers were dried over anhydrous Na₂SO₄, filtered and concentrated under reduced pressure. The residue was purified by column chromatography (silica gel, (PE/EtOAc=4/1-1/1) to give compound 9 as an off-white solid (1.4 g, yield 82%).

LC/MS (ESI) m/z: 383.8 [M+H]⁺.

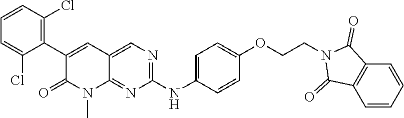 [SMILES: Cn1c(=O)c(-c2c(Cl)cccc2Cl)cc2cnc(Nc3ccc(OCCN4C(=O)c5ccccc5C4=O)cc3)nc21]

### 2-(2-(4-((6-(2,6-Dichlorophenyl)-8-methyl-7-oxo-7,8-dihydropyrido[2,3-d]pyrimidin-2-yl)amino)phenoxy)ethyl)isoindoline-1,3-dione (10)

A mixture of compound 4 (3.3 g, 11.9 mmol), compound 9 (0.92 g, 2.4 mmol) and TFA (1.1 g, 9.6 mmol) in 2-BuOH (20 mL) was stirred at 100° C. for 24 hours. The mixture was diluted with brine (100 mL) and extracted with DCM (200 mL×2). The combined organic layers were dried over anhydrous Na₂SO₄, filtered and concentrated in vacuum, the residue was purified by column chromatography (silica gel, DCM/MeOH=10/1) to give compound 10 as a white solid (690 mg, yield 49%).

LC/MS (ESI) m/z: 586.1 [M+H]⁺.

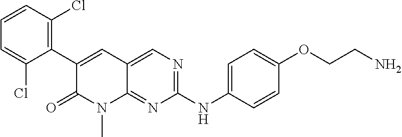 [SMILES: Cn1c(=O)c(-c2c(Cl)cccc2Cl)cc2cnc(Nc3ccc(OCCN)cc3)nc21]

### 2-((4-(2-Aminoethoxy)phenyl)amino)-6-(2,6-dichlorophenyl)-8-methylpyrido[2,3-d]pyrimidin-7(8H)-one (11)

A mixture of compound 10 (590 mg, 1.01 mmol) and N₂H₄·H₂O (503 mg, 10 mmol) in EtOH (20 mL) was heated to reflux for 16 hours. The mixture was concentrated under reduced pressure. The residue was diluted with water (100 mL) and extracted with EtOAc (100 mL×2). The combined organic layers were dried over anhydrous Na₂SO₄, filtered and concentrated under reduced pressure. The resulting residue was purified by column chromatography (silica gel, (DCM/MeOH=10/l) to give compound 11 as a yellow solid (290 mg, yield 63%).

LC/MS (ESI) m z: 456.1 [M+H]⁺.

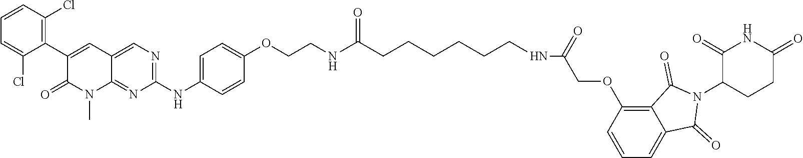 [SMILES: Cn1c(=O)c(-c2c(Cl)cccc2Cl)cc2cnc(Nc3ccc(OCCNC(=O)CCCCCCNC(=O)COc4cccc5c4C(=O)N(C4CCC(=O)NC4=O)C5=O)cc3)nc21]

### SB1-G-192

A mixture of compound 11 (109 mg, 0.24 mmol), compound 12 (1I0 mg, 0.22 mmol), HATU (28.4 mg, 0.07 mmol) and DIPEA (48.3 mg, 0.37 mmol) in DCM (3.0 mL) was stirred at RT for 4 hours, until LC/MS showed full conversion of starting material. The mixture was diluted with water (50 mL) and extracted with DCM (100 mL×2). The combined organic layers were dried over anhydrous Na₂SO₄, filtered and concentrated under reduced pressure. The resulting residue was purified by preparative HPLC (C18 column, CH₃CN/H₂O, containing 0.05% NH₄HCO₃) to give compound SB1-G-192 as an off-white solid (35.7 mg).

¹H NMR (DMSO-dc, 400 MHz): δ (ppm) 10.10 (br, 1H), 8.80 (s, 1H), 8.04 (t, J=5.6 Hz, 1H), 7.91 (t, J=5.6 Hz, 1H), 7.87 (s, 1H), 7.80 (dd, J=7.2 Hz, 8.4 Hz, 1H), 7.72 (d, J=8.4 Hz, 2H), 7.60 (s, 1H), 7.58 (s, 1H), 7.52-7.45 (m, 2H), 7.38 (d, J=8.4 Hz, 1H), 6.95 (d, J=8.8 Hz, 2H), 5.12 (dd. J=12.8 Hz, 5.2 Hz, 1H), 4.76 (s, 2H), 3.97 (t, J=5.6 Hz, 2H), 3.63 (s, 3H), 3.17-3.08 (m, 2H), 2.94-2.85 (m, 1H), 2.69-2.53 (m, 2H), 2.15-1.95 (m, 4H), 1.54-1.35 (m, 4H), 1.32-1.16 (m, 7H).

LC/MS (EST) m/z: 897.1 [M+H]⁺.

### Example 12: Synthesis of N-(4-((4-(6-(2-((2-(2,6-dioxopiperidin-3-yl)-1,3-dioxoisoindolin-4-yl)oxy)acetamido)hexyl)piperazin-1-yl)methyl)-3-(trifluoromethyl)phenyl)-3-(imidazo[1,2-b]pyridazin-3-ylethynyl)-4-methylbenzamide (SB1-G-188)

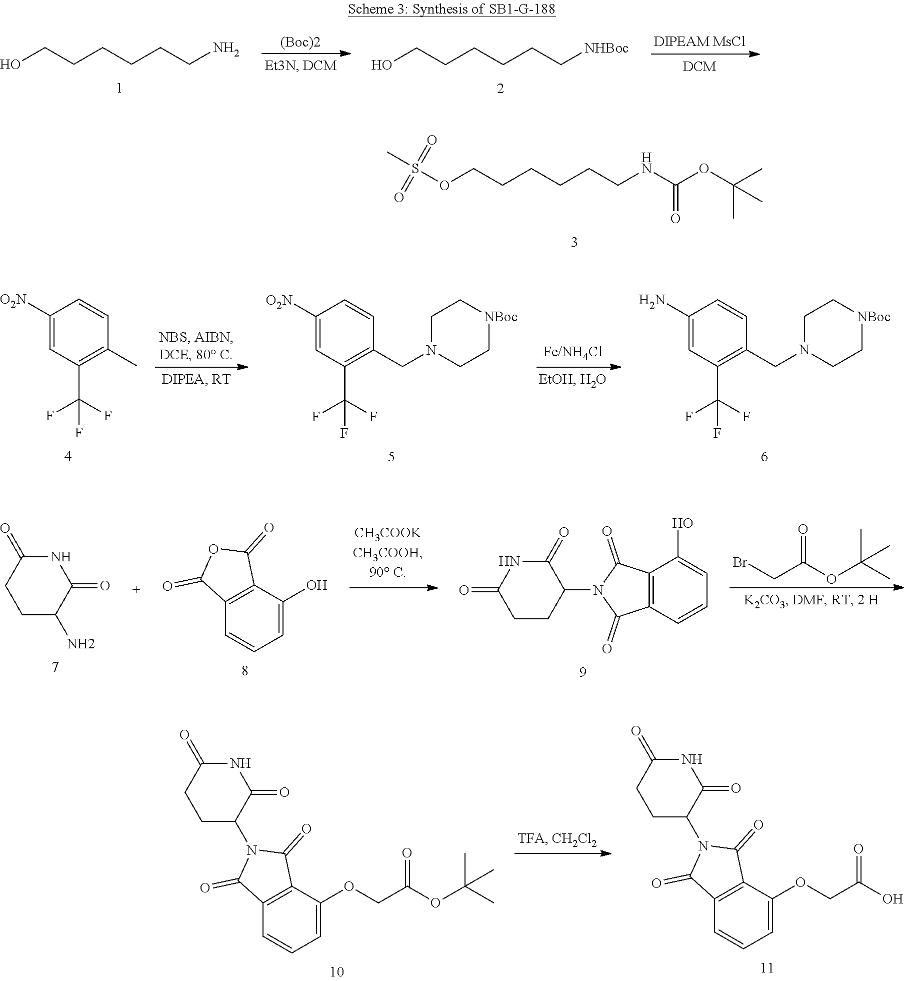

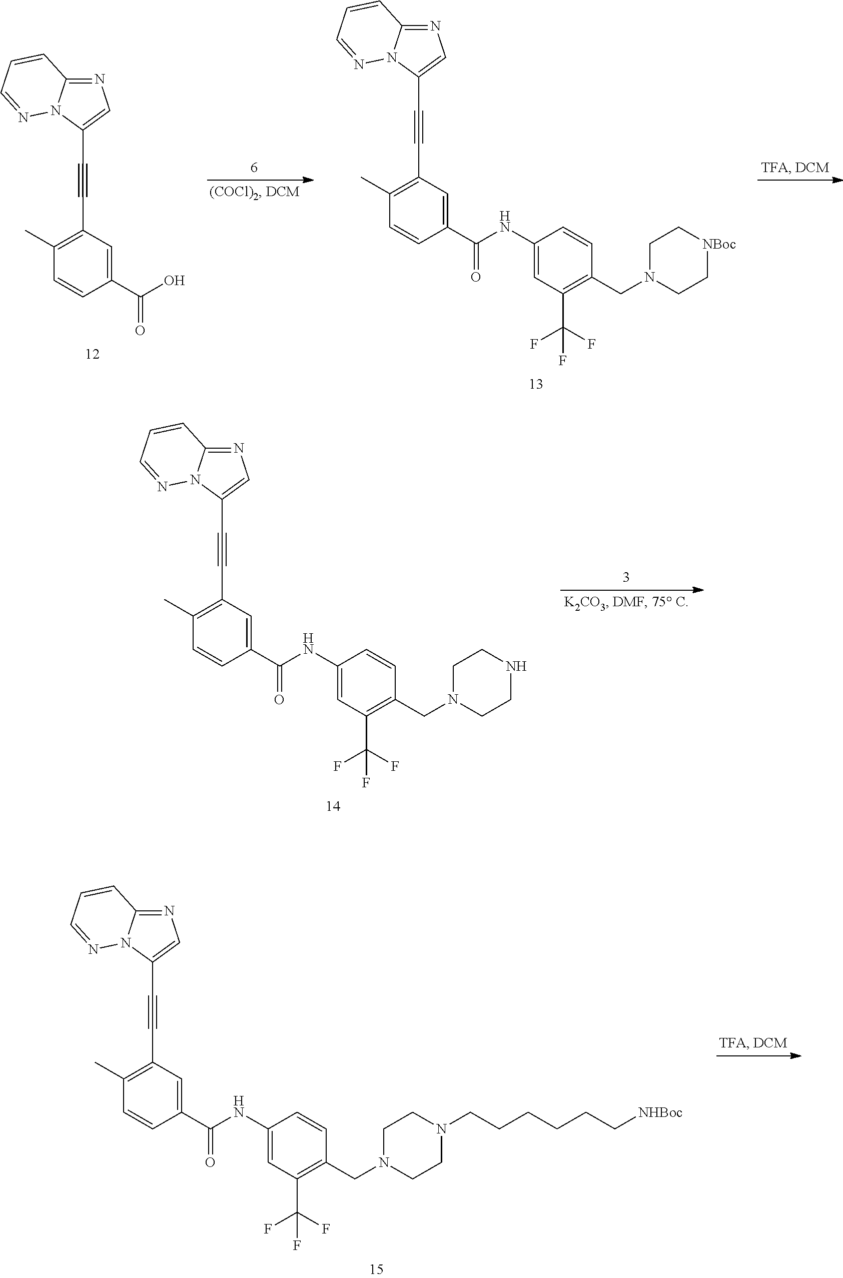 [SMILES: Cc1ccc(C(=O)O)cc1C#Cc1cnc2cccnn12 ; Cc1ccc(C(=O)Nc2ccc(CN3CCN(C(=O)OC(C)(C)C)CC3)c(C(F)(F)F)c2)cc1C#Cc1cnc2cccnn12 ; Cc1ccc(C(=O)Nc2ccc(CN3CCNCC3)c(C(F)(F)F)c2)cc1C#Cc1cnc2cccnn12 ; Cc1ccc(C(=O)Nc2ccc(CN3CCN(CCCCCCNC(=O)OC(C)(C)C)CC3)c(C(F)(F)F)c2)cc1C#Cc1cnc2cccnn12]

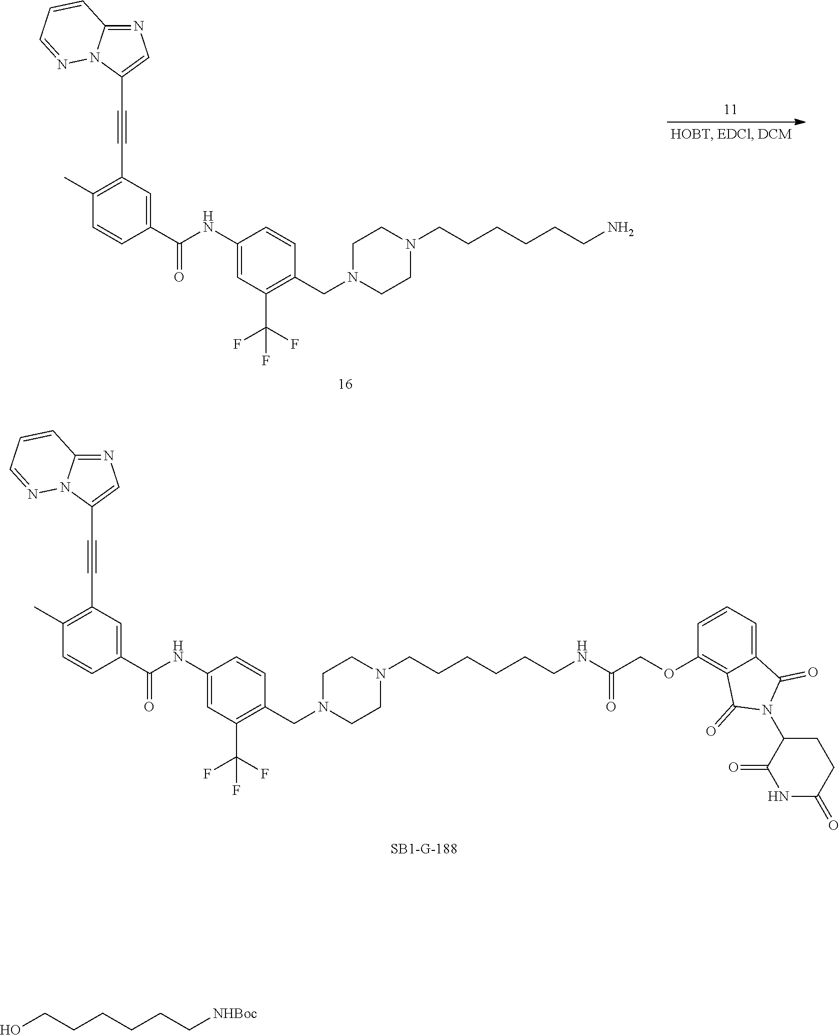 [SMILES: Cc1ccc(C(=O)Nc2ccc(CN3CCN(CCCCCCN)CC3)c(C(F)(F)F)c2)cc1C#Cc1cnc2cccnn12 ; Cc1ccc(C(=O)Nc2ccc(CN3CCN(CCCCCCNC(=O)COc4cccc5c4C(=O)N(C4CCC(=O)NC4=O)C5=O)CC3)c(C(F)(F)F)c2)cc1C#Cc1cnc2cccnn12 ; CC(C)(C)OC(=O)NCCCCCCO]

### tert-Butyl (6-hydroxyhexyl)carbamate (2)

To a mixture of 6-aminohexan-1-ol (2.0 g 17.0 mmol) and Et₃N (6.0 mL) in THF (15 mL) was added (Boc)₂O (5.6 g, 25.0 mmol) at 0° C. The mixture was stirred at RT for 16 hours and concentrated under reduced pressure. The resulting residue was purified by column chromatography (silica gel, MeOH/DCM=1/20) to afford compound 2 as a white solid (2.8 g, yield 77%).

LC/MS (ESI) m/z: 118.2 [M−100+H]⁺, 240.1 [M+Na]⁺.

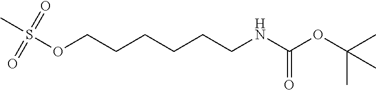 [SMILES: CC(C)(C)OC(=O)NCCCCCCOS(C)(=O)=O]

### 6-((tert-Butoxycarbonyl)amino)hexyl methanesulfonate (3)

MsCl (2.1 g, 18.6 mmol) was added dropwise to a mixture of compound 2 (2.7 g, 12.4 mmol) and DIPEA (4.2 mL) in DCM (30 mL) at 0° C. The mixture was stirred at RT for 4 hours and then diluted with saturated aqueous NaHCO₃(100 mL). The mixture was extracted with DCM (100 mL×2). The combined organic layers were washed with brine (100 mL×2), dried over anhydrous Na₂SO₄, filtered, and concentrated under reduced pressure to obtain crude compound 3 as a yellow oil (2.5 g, yield 69%).

LC/MS (ESI) m/z: 196.1 [M−100+H]⁺, 318.0 [M+Na]⁺.

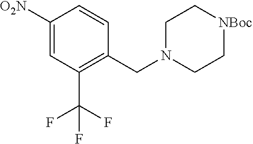 [SMILES: CC(C)(C)OC(=O)N1CCN(Cc2ccc([N+](=O)[O-])cc2C(F)(F)F)CC1]

### tert-Butyl 4-(4-nitro-2-(trifluoromethyl)benzyl)piperazine-1-carboxylate (5)

A mixture of 1-methyl-4-nitro-2-(trifluoromethyl)benzene (4.0 g, 19.5 mmol), NBS (3.8 g, 21.4 mmol), and AIBN (639 mg, 3.9 mmol) in DCE (30.0 mL) was stirred at 80° C. for 16 hours. The mixture was cooled to RT before addition of tert-butyl piperazine-1-carboxylate (4.7 g, 25.3 mmol) and DIPEA (6.7 mL). The resulting mixture was stirred at RT for 2 hours, diluted with water (200 1454 mL), and extracted with DCM (100 mL×2). The combined organic layers were washed with brine (100 mL×2), dried over anhydrous Na₂SO₄, filtered and concentrated under reduced pressure. The resulting residue was purified by column chromatography (silica gel, EtOAc/PE=1/5) to obtain compound 5 as a white solid (4.5 g, yield 60%).

LC/MS (ESI) m/z: 390.0 [M+H]⁺.

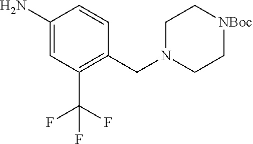 [SMILES: CC(C)(C)OC(=O)N1CCN(Cc2ccc(N)cc2C(F)(F)F)CC1]

### tert-Butyl 4-(4-amino-2-(trifluoromethyl)benzyl)piperazine-1-carboxylate (6)

A mixture of compound 5 (4.4 g, 11.3 mmol), Fe (3.16 g, 56.5 mmol) and NH₄Cl (3.16 g, 56.5 mmol) in EtOH (30.0 mL) and H₂O (4.0 mL) was stirred at 80° C. for 3 hour. The mixture was filtered, and the filtrate was concentrated under reduced pressure. The resulting residue was diluted with H₂O (2(X) mL) and extracted with DCM (150 mL×2). The combined organic layers were dried over anhydrous Na₂SO₄, filtered, and concentrated under reduced pressure. The resulting residue was purified by column chromatography (silica gel, MeOH/DCM=1/20) to obtain compound 6 as a yellow oil (3.5 g, yield 87.5%).

LC/MS (ESI) m/z: 360.0 [M+H]⁺.

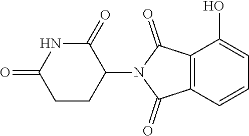 [SMILES: O=C1CCC(N2C(=O)c3cccc(O)c3C2=O)C(=O)N1]

### 2-(2,6-Dioxopiperidin-3-yl)-4-hydroxyisoindoline-1,3-dione (9)

A mixture of 3-aminopiperidine-2,6-dione (8.0 g, 48.7 mmol), 4-hydroxyisobenzofuran-1,3-dione (8.0 g, 48.7 mmol) and CH₃COOK (14.3 g, 146.3 mmol) in CH₃COOH (30.0 mL) was stirred at 90° C. for 16 hours. The mixture was cooled to RT and diluted with H₂O (200 mL), and filtered. The resulting precipitate was collected and air dried to obtain compound 9 as a gray solid (10.0 g, yield 77%).

LC/MS (ESI) m/z: 275.2 [M+H]⁺.

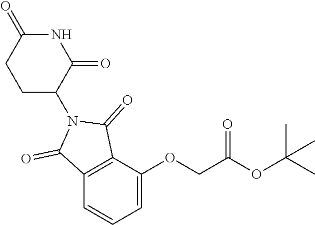 [SMILES: CC(C)(C)OC(=O)COc1cccc2c1C(=O)N(C1CCC(=O)NC1=O)C2=O]

### tert-Butyl 2-((2-(2,6-dioxopiperidin-3-yl)-1,3-dioxoisoindolin-4-yl)oxy)acetate (10)

tert-Butyl 2-bromoacetate (4.98 g, 25.5 mmol) was added dropwise to a mixture of compound 9(7.0 g, 25.5 mmol) and K₂CO₃ (5.27 g, 38.25 mmol) in DMF (30 mL). The mixture was stirred at RT for 4 hours, diluted with EtOAc (300 mL), and washed with brine (100 mL×2). The organic layer was dried over anhydrous Na₂SO₄, filtered and concentrated under reduced pressure. The resulting residue was slurried in a mixture of EtOAc/PE (1/8, 100 mL), filtered, and dried under vacuum to obtain compound 10 as a white solid (8.7 g, yield 87.8%).

LC/MS (ESI) m/z: 333.0 [M−56+H]⁺.

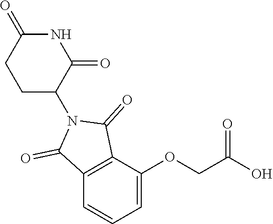 [SMILES: O=C(O)COc1cccc2c1C(=O)N(C1CCC(=O)NC1=O)C2=O]

### 2-((2-(2,6-Dioxopiperidin-3-yl)-1,3-dioxoisoindolin-4-yl)oxy)acetic add (11)

A mixture of compound 10 (7.7 g, 19.8 mmol) and TFA (20.0 mL) in DCM (20.0 mL) was stirred at RT for 2 hours. The mixture was concentrated under reduced pressure to obtain crude compound 11 as a white solid (6.0 g, yield 92%).

LC/MS (ESI) m/z: 333.3 [M+H]⁺.

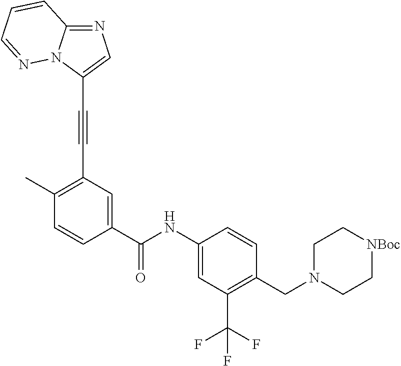 [SMILES: Cc1ccc(C(=O)Nc2ccc(CN3CCN(C(=O)OC(C)(C)C)CC3)c(C(F)(F)F)c2)cc1C#Cc1cnc2cccnn12]

### tert-Butyl 4-(4-(3-(imidazo[1,2-b]pyridazin-3-ylethynyl)-4-methylbenzamido)-2-(trifluoromethyl)benzyl)piperazine-1-carboxylate (13)

To a solution of compound 12 (300 mg, 1.08 mmol) in DCM (50 mL) was added DMF (0.1 mL). (COCl)₂ (0.5 mL) was added dropwise to the mixture at 0° C., and the reaction was stirred at RT for 2 hours. The crude mixture was concentrated under reduced pressure. The resulting residue was redissolved in DCM (10 mL) and added dropwise to a solution of compound 6 (466 mg, 1.29 mmol) and DIPEA (1.0 mL) in DCM (40 mL) at 0° C. The resulting mixture was stirred at RT for 2 hours, diluted with brine (100 mL) and extracted with DCM (100 mL×2). The combined organic layers were dried over anhydrous Na₂SO₄, filtered, and concentrated under reduced pressure. The crude residue was purified by preparative HPLC to obtain compound 13 as a yellow solid (240 mg, yield 36%).

LC/MS (ESI) m/z: 619.0 [M+H]⁺.

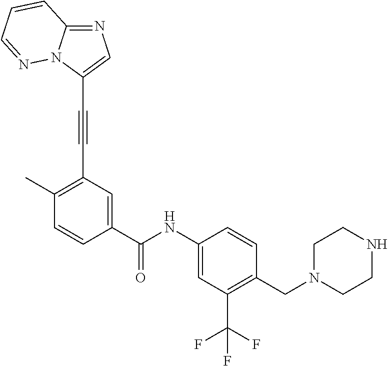 [SMILES: Cc1ccc(C(=O)Nc2ccc(CN3CCNCC3)c(C(F)(F)F)c2)cc1C#Cc1cnc2cccnn12]

### 3-(Imidazo[1,2-b]pyridazin-3-ylethynyl)-4-methyl-N-(4-(piperazin-1-ylmethyl)-3-(trifluoromethyl)phenyl)benzamide (14)

A solution of compound 13 (100 mg, 0.16 mmol) and TFA (2.0 mL) in DCM (2.0 mL) was stirred at RT for 1 hour. The mixture was concentrated under reduced pressure, and the resulting residue was diluted with water (50 mL). The pH was adjusted to 8 before extraction of the mixture with EtOAc (50 mL×2). The combined organic layers were washed with brine (50 mL×2), dried over anhydrous Na₂SO₄, filtered, and concentrated in vacuum to obtain crude compound 14 as a yellow oil (80 mg, yield 96%).

LC/MS (ESI) m/z: 519.0 [M+H]⁺.

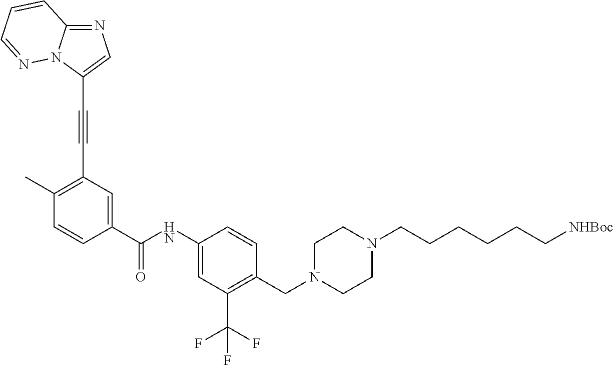 [SMILES: Cc1ccc(C(=O)Nc2ccc(CN3CCN(CCCCCCNC(=O)OC(C)(C)C)CC3)c(C(F)(F)F)c2)cc1C#Cc1cnc2cccnn12]

### tert-Butyl (6-(4-(4-(3-(imidazo[1,2-b]pyridazin-3-ylethynyl)-4-methylbenzamido)-2-(trifluoromethyl)benzyl)piperazin-1-yl)hexyl)carbamate (15)

A mixture of compound 14 (80 mg, 0.15 mmol), compound 3 (70 mg, 0.23 mmol) and K₂CO₃ (42 mg, 0.3 mmol) in DMF (6.0 mL) was stirred at 90° C. for 16 hours. The mixture was diluted with EtOAc (100 mL), washed with brine (50 mL×2). The organic layer was dried over anhydrous Na₂SO₄, filtered, and concentrated under reduced pressure. The resulting residue was purified by column chromatography (silica gel, MeOH/DCM=1/20) to obtain compound 15 as a brown oil (80 mg, yield 72.7%).

LC/MS (ESI) m/z.: 718.1 [M+H]⁺.

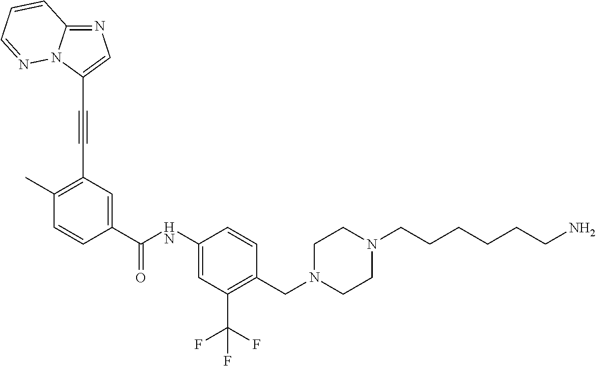 [SMILES: Cc1ccc(C(=O)Nc2ccc(CN3CCN(CCCCCCN)CC3)c(C(F)(F)F)c2)cc1C#Cc1cnc2cccnn12]

### N-(4-((4-(6-Aminohexyl)piperazin-1-yl)methyl)-3-(trifluoromethyl)phenyl)-3-(imidazo[1,2-b]pyridazin-3-ylethynyl)-4-methylbenzamide (16)

A solution of compound 15 (80 mg, 0.11 mmol) and TFA (1.0 mL) in DCM (2.0 mL) was stirred at RT for 1 hour. The mixture was concentrated under reduced pressure, and the resulting residue was diluted with water (50 mL). The pH was adjusted to 8 before extraction of the mixture with EtOAc (50 mL×2). The combined organic layers were washed with brine (50 mL×2), dried over anhydrous Na₂SO₄, filtered, and concentrated in vacuum to obtain crude compound 16 as a brown oil (60 mg, yield 88%).

LC/MS (ESI) m/z: 618.0 [M+H]⁺.

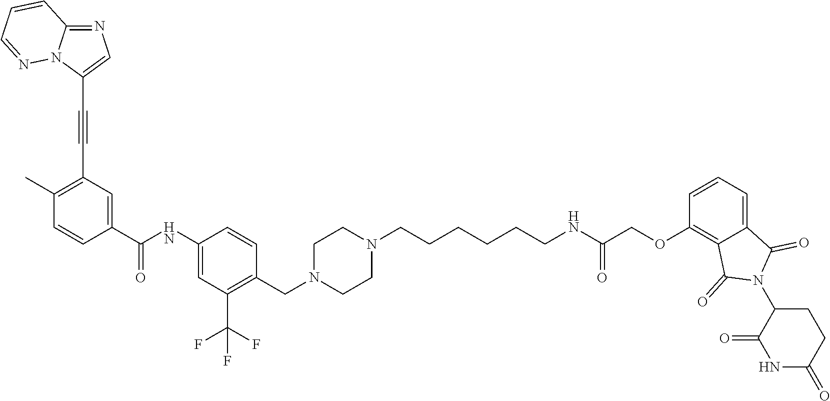 [SMILES: Cc1ccc(C(=O)Nc2ccc(CN3CCN(CCCCCCNC(=O)COc4cccc5c4C(=O)N(C4CCC(=O)NC4=O)C5=O)CC3)c(C(F)(F)F)c2)cc1C#Cc1cnc2cccnn12]

### SB1-G-188

A solution of compound 11(42 mg, 0.12 mmol) and HOBt (20 mg, 0.14 mmol) in DCM (5.0 mL) was stirred at RT for 15 minutes, 1-Ethyl-3-(3-dimethylaminopropyl)carbodiimide (EDCI) (37 mg, 0.19 mmol) was added to the mixture, and the reaction was stirred at RT for another 15 minutes before the addition of compound 16 (60 mg, 0.09 mmol). The resulting mixture was stirred at RT for 2 hours, diluted with water (100 mL), and extracted with DCM (100 mL×2). The combined organic layers were dried over anhydrous Na₂SO₄, filtered and concentrated under reduced pressure. The resulting residue was purified by preparative HPLC (C18 column, CH3CN/H₂O, containing 0.05% NH₄HCO₃) to obtain SB1-G-188 as a light yellow solid (10.4 mg, yield 11.1%).

¹H NMR (CDCl₃, 400 MHz): δ (ppm) 8.49 (d, J=4.0 Hz, 1H), 8.17 (s, 1H), 8.08-8.06 (m, 2H), 7.99 (d, J=9.2 Hz, 1H), 7.91-7.89 (m, 2H), 7.82 (d, J=7.2 Hz, 1H), 7.77-7.70 (m, 2H), 7.58-7.53 (m, 2H), 7.40 (d, J=8.4 Hz, 1H), 7.17-7.12 (m, 2H), 4.94-4.91 (m, 1H), 4.67-4.54 (dd, J=13.6, 37.2 Hz, 2H), 3.61 (s, 2H), 3.51-3.46 (m, 1H), 3.27-3.23 (m, 1H), 2.86-2.5 (m, 8H), 2.27-2.48 (m, 3H), 2.17-2.12 (m, 2H), 1.66-1.47 (m, 9H), 1.43-1.25 (m, 4H).

LC/MS (ESI) m/z: 932.0 [M+H]⁺.

### Example 13: Synthesis of N-(4-((4-(1-((2-(2,6-dioxopiperidin-3-yl)-1,3-dioxoisoindolin-4-yl)oxy)-2-oxo-6,9,12-trioxa-3-azapentadecan-15-yl)piperazin-1-yl)methyl)-3 (trifluoromethyl)phenyl)-3-(imidazo[1,2-b]pyridazin-3-ylethynyl)-4-methylbenzamide (SB1-G-187)

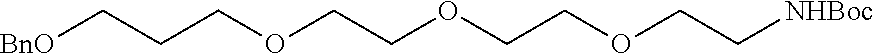 [SMILES: CC(C)(C)OC(=O)NCCOCCOCCOCCCOCc1ccccc1]

### tert-Butyl (1-phenyl-2,6,9,12-tetraoxatetradecan-14-yl)carbamate (3)

To a mixture of tert-butyl (2-(2-(2-hydroxyethoxy)ethoxy)ethyl)carbamate (1.5 g, 6.0 mmol) in DMF (15 mL) was slowly added NaH (1.2 g, 30.0 mmol) (in portions) at 0° C. The reaction was stirred at 0° C. 1 hour before the addition of ((3-Bromopropoxy)methyl)benzene (1.5 g, 6.6 mmol). The resulting mixture was stirred at RT for 16 hours, diluted with brine (200 mL) and extracted with DCM (150 mL×2). The combined organic layers were dried over anhydrous Na₂SO₄, filtered and evaporated under reduced pressure. The residue was purified by column chromatography (silica gel, EtOAc/PE=1/5) and preparative HPLC (C18 column, CH3CN/H₂O, containing 0.05% TFA) to obtain compound 3 as a colorless oil (380 mg, yield 15%).

LC/MS (ESI) m/z: 420.1 [M+Na]⁺, 298.1 [M−100+H]⁺.

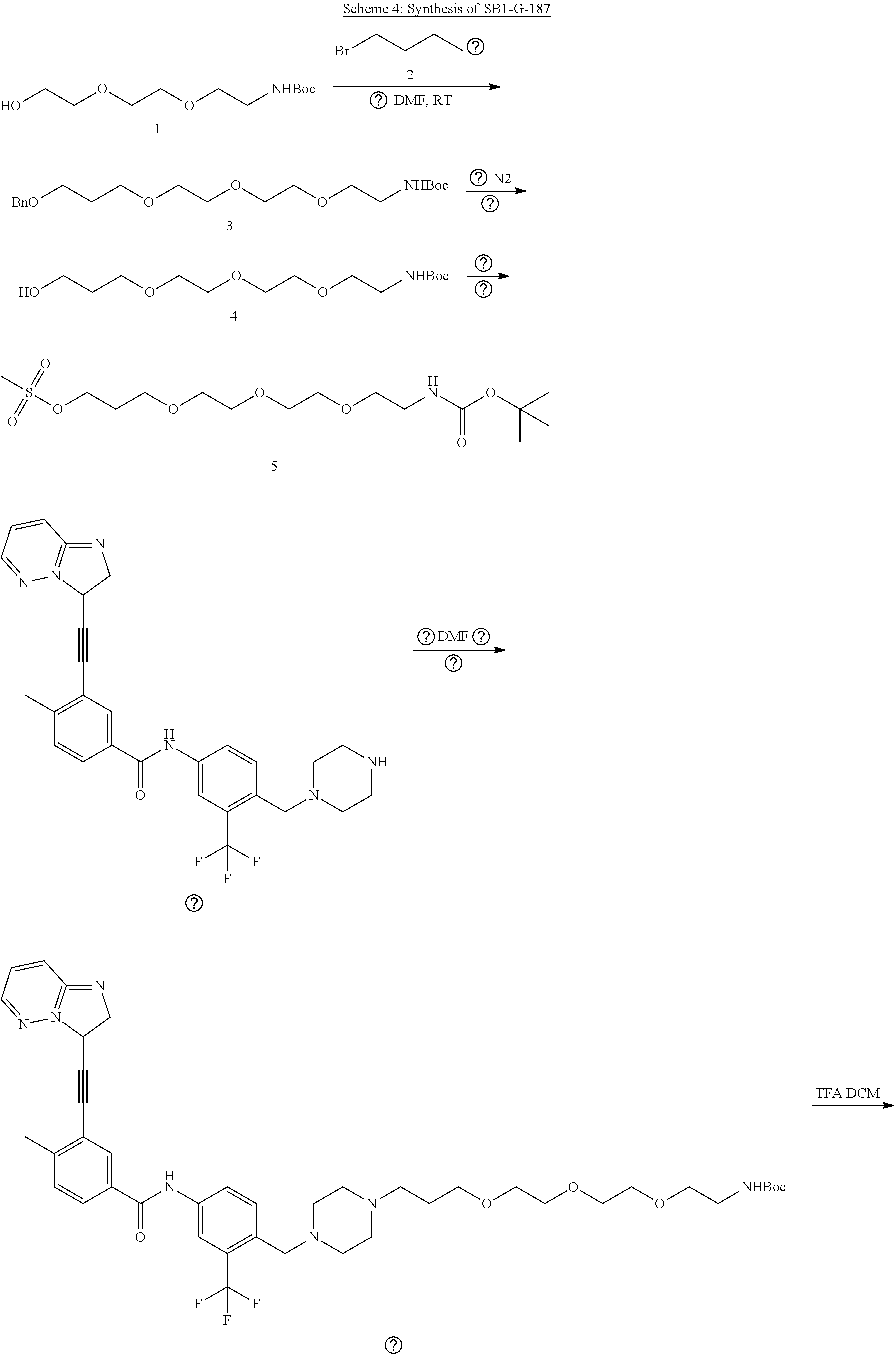

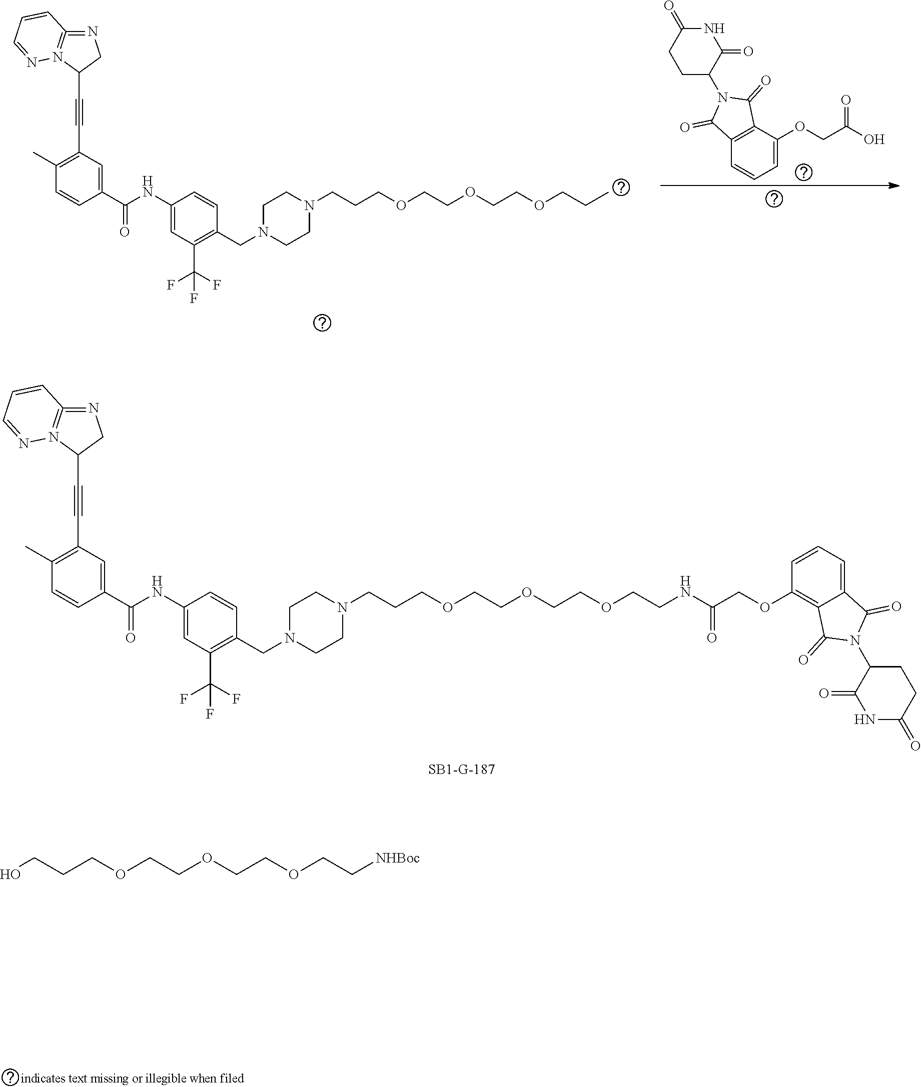

### tert-Butyl (2-(2-(2-(3-hydroxypropoxy)ethoxy)ethoxy)ethyl)carbamate (4)

A mixture of compound 3 (350 mg, 0.8 mmol) and Pd/C (10%, 100 mg) in EtOH (10.0 mL) was stirred at RT under H₂ (1 atm) for 2 hours. The mixture was filtered through Celite®, and the filtrate was concentrated under reduce pressure to obtain crude compound 4 as a colorless oil (280 mg, yield 90%).

LC/MS (ESI) m/z: 208.1 [M−100+H]⁺, 330.1 [M+Na]⁺.

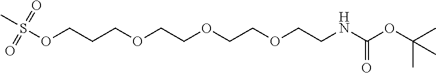 [SMILES: CC(C)(C)OC(=O)NCCOCCOCCOCCCOS(C)(=O)=O]

### 2,2-Dimethyl-4-oxo-3,8,11,14-tetraoxa-5-azaheptadecan-17-yl methanesulfonate (5)

To a solution of compound 4 (250 mg, 0.81 mmol) and DIPEA (0.3 mL) in DCM (8 mL) was added MsCl (93 mg, 0.81 mmol) at 0° C. The mixture was stirred at RT for 2 hours, diluted with saturated NaHCO₃ solution (200 mL), and extracted with DCM (150 mL×2). The combined organic layers were washed with brine (100 mL×2), dried over anhydrous Na₂SO₄, filtered and evaporated under reduced pressure to obtain crude compound 5 as a brown oil (300 mg, yield 95%).

LC/MS (ESI) m/z: 286.0 [M−100+H]⁺, 408.0 [M+Na]⁺.

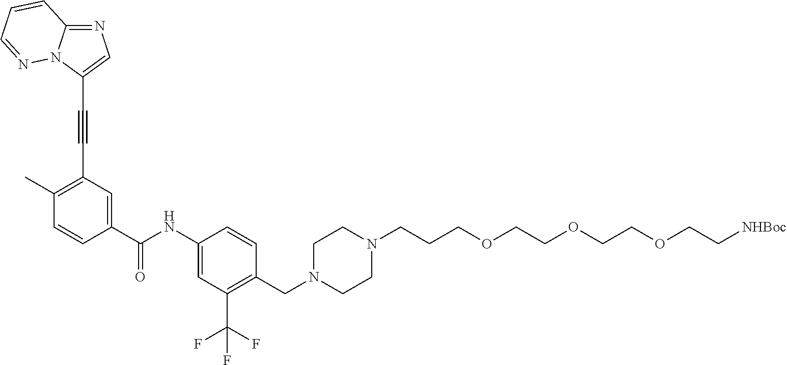 [SMILES: Cc1ccc(C(=O)Nc2ccc(CN3CCN(CCCOCCOCCOCCNC(=O)OC(C)(C)C)CC3)c(C(F)(F)F)c2)cc1C#Cc1cnc2cccnn12]

### tert-Butyl (2-(2-(2-(3-(4-(4-(3-(imidazo[1,2-b]pyridazin-3-ylethynyl)-4-methylbenzamido)-2-(trifluoromethyl)benzyl)piperazin-1-yl)propoxy)ethoxy)ethoxy)ethyl)carbamate (7)

A mixture of compound 6 (90 mg, 0.17 mmol), compound 5 (100 mg, 0.26 mmol) and K₂CO₃ (48 mg, 0.34 mmol) in DMF (6.0 mL) was stirred at 70° C. for 16 hours. The mixture was diluted with EtOAc (100 mL), washed with brine (50 mL×2), dried over anhydrous Na₂SO₄, filtered, and concentrated under reduced pressure. The residue was purified by column chromatography (silica gel, MeOH/DCM=1/20) to provide compound 7 as a yellow oil (70 mg, yield 40.4%).

LC/MS (ESI) m/z: 808.1 [M+H]⁺.

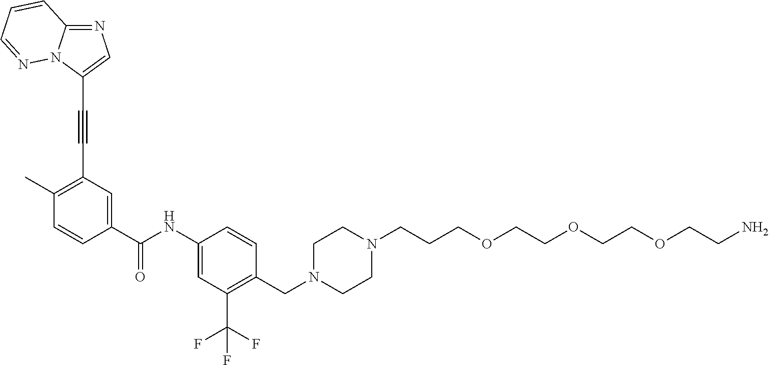 [SMILES: Cc1ccc(C(=O)Nc2ccc(CN3CCN(CCCOCCOCCOCCN)CC3)c(C(F)(F)F)c2)cc1C#Cc1cnc2cccnn12]

### N-(4-((4-(3-(2-(2-(2-Aminoethoxy)ethoxy)ethoxy)propyl)piperazin-1-yl)methyl)-3-(trifluoromethyl)phenyl)-3-(imidazo[1,2-b]pyridazin-3-ylethynyl)-4-methylbenzamide (8)

A solution of compound 7 (70 mg, 0.08 mmol) and TFA (2.0 mL) in DCM (2.0 mL) was stirred at RT for 1 hour. The mixture was concentrated under reduced pressure, and the resulting residue was diluted with water (50 mL). The pH was adjusted to 8 before extraction of the mixture with EtOAc (50 mL×2). The combined organic layers were washed with brine (50 mL×2), dried over anhydrous Na₂SO₄, filtered, and concentrated in vacuum to obtain crude compound 8 as a brown oil (80 mg, crude).

LC/MS (ESI) m/z: 708.1 [M+H]⁺.

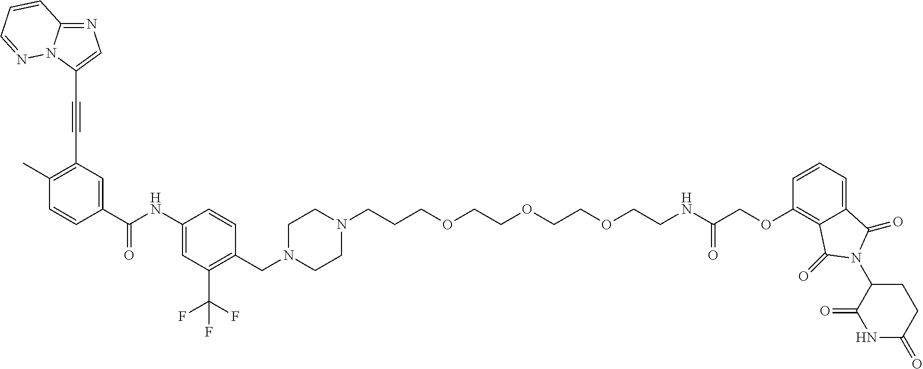 [SMILES: Cc1ccc(C(=O)Nc2ccc(CN3CCN(CCCOCCOCCOCCNC(=O)COc4cccc5c4C(=O)N(C4CCC(=O)NC4=O)C5=O)CC3)c(C(F)(F)F)c2)cc1C#Cc1cnc2cccnn12]

### SB1-G-187

A solution of 2-((2-(2,6-dioxopiperidin-3-yl)-1,3-dioxoisoindolin-4-yl)oxy)acetic acid (43 mg, 0.12 mmol) and HOBt (20 mg, 0.14 mmol) in DCM (5.0 mL) as stirred at RT for 15 minutes. EDCI was added to the mixture, and the reaction was stirred at RT for another 15 minutes before the addition of compound 8 (70 mg, 0.09 mmol). The resulting mixture was stirred at RT for 2 hours, diluted with water (100 mL), and extracted with DCM (100 mL×2). The combined organic layers were dried over anhydrous Na₂SO₄, filtered and concentrated under reduced pressure. The resulting residue was purified by preparative HPLC (C18 column, CH₃CN/H₂O, containing 0.05% NH₄HCO₃) to obtain SB1-G-187 as a light yellow solid (38 mg, yield 37.6%).

¹H NMR (CDCl₃, 400 MHz): δ (ppm) 8.49 (dd, J=1.2, 4.4 Hz, 1H), 8.32-8.28 (m, 1H), 8.08-8.07 (m, 2H), 7.98 (d, J=8.8 Hz, 1H), 7.92-7.88 (m, 2H), 7.83 (dd, J=2.0, 8.0 Hz, 1H), 7.76-7.67 (m, 3H), 7.52 (d, J=7.2 Hz, 1H), 7.39 (d, J=8.0 Hz, 1H), 7.17-7.12 (m, 2H), 4.96-4.92 (m, 1H), 4.64 (s, 2H), 3.69-3.63 (m, 10H), 3.60-3.56 (m, 4H), 3.50 (t, J=6.4 Hz, 2H), 2.90-2.72 (m, 3H), 2.64 (s, 3H), 2.45-2.44 (m, 3H), 2.17-2.11 (m, 1H), 1.81-1.66 (m, 9H).

LC/MS (ESI) m/z: 511.6 [M/2+H]⁺.

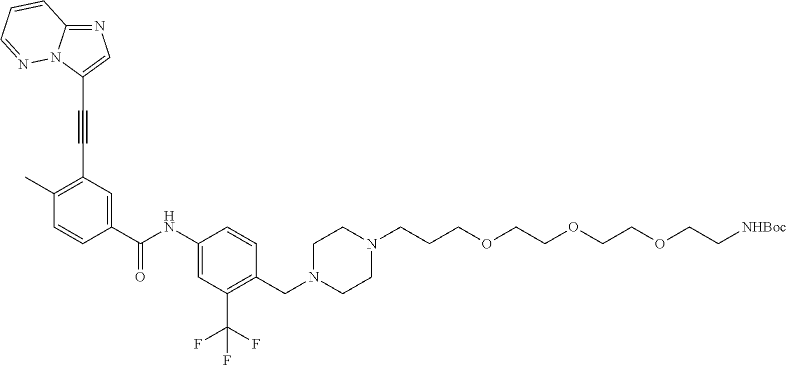 [SMILES: Cc1ccc(C(=O)Nc2ccc(CN3CCN(CCCOCCOCCOCCNC(=O)OC(C)(C)C)CC3)c(C(F)(F)F)c2)cc1C#Cc1cnc2cccnn12]

### tert-Butyl (2-(2-(2-(3-(4-(4-(3-(imidazo[1,2-b]pyridazin-3-ylethynyl)-4-methylbenzamido)-2-(trifluoromethyl)benzyl)piperazin-1-yl)propoxy)ethoxy)ethoxy)ethyl)carbamate (7)

A mixture of compound 6 (90 mg, 0.17 mmol), compound 5 (100 mg, 0.26 mmol) and K2C03 (48 mg, 0.34 mmol) in DMF (6.0 mL) was stirred at 70° C. for 16 hours. The mixture was diluted with EtOAc (100 mL), washed with brine (50 mL×2), dried over anhydrous Na₂SO₄, filtered, and concentrated under reduced pressure. The residue was purified by column chromatography (silica gel, MeOH/DCM=1/20) to provide compound 7 as a yellow oil (70 mg, yield 40.4%).

LC/MS (ESI) mm/z: 808.1 [M+H]⁺.

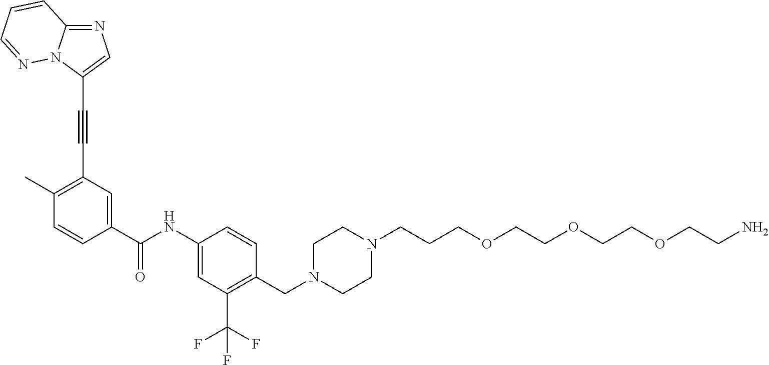 [SMILES: Cc1ccc(C(=O)Nc2ccc(CN3CCN(CCCOCCOCCOCCN)CC3)c(C(F)(F)F)c2)cc1C#Cc1cnc2cccnn12]

### N-(4-((4-(3-(2-(2-(2-Aminoethoxy)ethoxy)ethoxy)propyl)piperazin-1-yl)methyl)-3-(trifluoromethyl)phenyl)-3-(imidazo[1,2-b]pyridazin-3-ylethynyl)-4-methylbenzamide (8)

A solution of compound 7 (70 mg, 0.08 mmol) and TFA (2.0 mL) in DCM (2.0 mL) was stirred at RT for 1 hour. The mixture was concentrated under reduced pressure, and the resulting residue was diluted with water (50 mL). The pH was adjusted to 8 before extraction of the mixture with EtOAc (50 mL×2). The combined organic layers were washed with brine (50 mL×2), dried over anhydrous Na₂SO₄, filtered, and concentrated in vacuum to obtain crude compound 7 as a brown oil (80 mg, crude).

LC/MS (ESI) m/z: 708.1 [M+H]⁺.

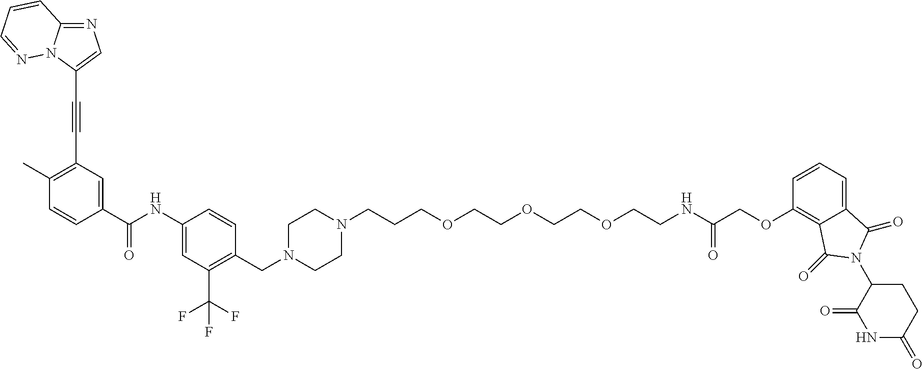 [SMILES: Cc1ccc(C(=O)Nc2ccc(CN3CCN(CCCOCCOCCOCCNC(=O)COc4cccc5c4C(=O)N(C4CCC(=O)NC4=O)C5=O)CC3)c(C(F)(F)F)c2)cc1C#Cc1cnc2cccnn12]

### SB1-G-187

A solution of 2-((2-(2,6-dioxopiperidin-3-yl)-1,3-dioxoisoindolin-4-yl)oxy)acetic acid (43 mg, 0.12 mmol) and HOBt (20 mg, 0.14 mmol) in DCM (5.0 mL) as stirred at RT for 15 minutes. EDCI was added to the mixture, and the reaction was stirred at RT for another 15 minutes before the addition of compound 8 (70 mg, 0.09 mmol). The resulting mixture was stirred at RT for 2 hours, diluted with water (100 mL), and extracted with DCM (100 mL×2). The combined organic layers were dried over anhydrous Na₂SO₄, filtered and concentrated under reduced pressure. The resulting residue was purified by preparative HPLC (C18 column, CH₃CN/H₂O, containing 0.05% NH₄HCO₃) to obtain SB1-G-187 as a light yellow solid (38 mg, yield 37.6%).

¹H NMR (CDCl₃, 400 MHz): δ (ppm) 8.49 (dd, J=1.2, 4.4 Hz, 1H), 8.32-8.28 (m, 1H), 8.08-8.07 (m, 2H), 7.98 (d, J=8.8 Hz, 1H), 7.92-7.88 (m, 2H), 7.83 (dd, J=2.0, 8.0 Hz, 1H), 7.76-7.67 (m, 3H), 7.52 (d, J=7.2 Hz, 1H), 7.39 (d, J=8.0 Hz, 1H), 7.17-7.12 (m, 2H), 4.96-4.92 (m, 1H), 4.64 (s, 2H), 3.69-3.63 (m, 10H), 3.60-3.56 (m, 4H), 3.50 (t, J=6.4 Hz, 2H), 2.90-2.72 (m, 3H), 2.64 (s, 3H), 2.45-2.44 (m, 3H), 2.17-2.11 (m, 1H), 1.81-1.66 (m, 9H).

LC/MS (ESI) m/z: 511.6 [M/2+H]⁺.

### Example 14: Synthesis of N-(2-(2-(4-(4-((5-chloro-4-((2-(isopropylsulfonyl)phenyl)amino)pyrimidin-2-yl)amino)phenyl)piperazin-1-yl)ethoxy)ethyl)-2-((2-(2,6-dioxopiperidin-3-yl)-1.3-1636 dioxoisoindolin-4-ylamino)acetamide (SK-3-91)

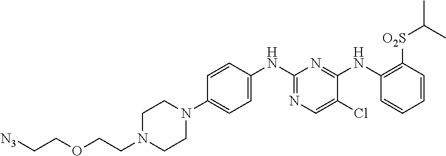 [SMILES: CC(C)S(=O)(=O)c1ccccc1Nc1nc(Nc2ccc(N3CCN(CCOCCN=[N+]=[N-])CC3)cc2)ncc1Cl]

### N2-(4-(4-(2-(2-Azidoethoxy)ethyl)piperazin-1-yl)phenyl)-5-chloro-N4-(2-(isopropylsulfonyl)phenyl)pyrimidine-2,4-diamine (3)

Intermediate 1 (in scheme 5, below) was prepared according to literature (Huang et al., Cell Chem. Biol. 25:88-99 (2018)). To a solution of 1 (590 mg, 1.0 mmol) in DCM (18 mL) was added TFA (1.8 mL), and the mixture was stirred at RT for 2 hours. The reaction mixture was concentrated under reduced pressure, and resulting residue was redissolved in acetonitrile (5 mL) before the addition of commercially available intermediate 2 (300 mg, 1.2 mmol) and potassium carbonate (414 mg, 3.0 mmol). The mixture was stirred overnight at 80° C. The reaction was allowed to cool to RT and then diluted with DCM (50 mL). The resulting mixture was filtered, and the filtrate was concentrated under reduced pressure and purified by column chromatograph (dichloromethane:methanol=10:1) to obtain compound 3 as a colorless oil (446 mg, yield 74%).

LC/MS (ESI) m/z: 600 [M+H]⁺.

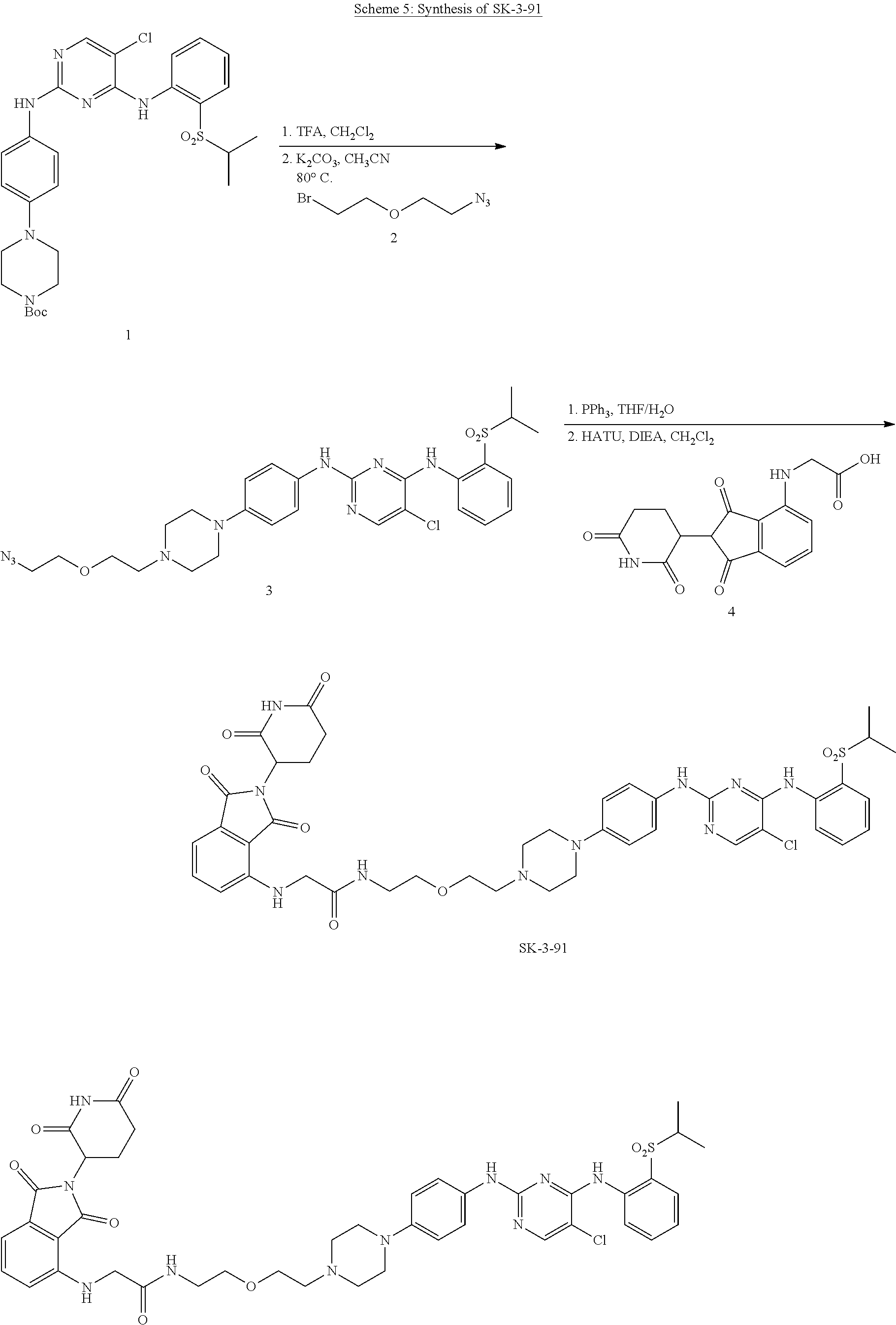 [SMILES: CC(C)S(=O)(=O)c1ccccc1Nc1nc(Nc2ccc(N3CCN(C(=O)OC(C)(C)C)CC3)cc2)ncc1Cl ; [N-]=[N+]=NCCOCCBr ; CC(C)S(=O)(=O)c1ccccc1Nc1nc(Nc2ccc(N3CCN(CCOCCN=[N+]=[N-])CC3)cc2)ncc1Cl ; O=C(O)CNc1cccc2c1C(=O)C(C1CCC(=O)NC1=O)C2=O ; CC(C)S(=O)(=O)c1ccccc1Nc1nc(Nc2ccc(N3CCN(CCOCCNC(=O)CNc4cccc5c4C(=O)N(C4CCC(=O)NC4=O)C5=O)CC3)cc2)ncc1Cl ; CC(C)S(=O)(=O)c1ccccc1Nc1nc(Nc2ccc(N3CCN(CCOCCNC(=O)CNc4cccc5c4C(=O)N(C4CCC(=O)NC4=O)C5=O)CC3)cc2)ncc1Cl]

### SK-3-91

Intermediate 4 was prepared according to literature (Huang et al., Cell Chem. Biol. 25:88-99 (2018)). To a solution of intermediate 3 (30 mg, 0.05 mmol) in THF (4.5 mL) and water (0.45 mL) was added triphenylphosphine (16 mg, 0.06 mmol) under a nitrogen atmosphere. The reaction mixture was stirred overnight and then concentrated under reduced pressure. To the obtained crude oil in anhydrous DCM (3 mL) was added intermediate 4 (18 mg, 0.055 mmol), HATU, and DIEA (27 μL, 0.15 mmol). The reaction mixture was stirred for 2 hours, concentrated under reduced pressure, and purified by preparative HPLC to give SK-3-91 as a yellow oil (35 mg, trifluoroacetate salt, yield 69%).

¹H NMR (400 MHz, Methanol-d₄, TFA salt) δ 8.59 (d, J=8.4 Hz, 1H), 8.16 (s, 1H), 7.92 (dd, J=8.0, 1.6 Hz, 1H), 7.69 (t, J=8.0 Hz, 1H), 7.58 (dd, J=8.5, 7.2 Hz, 1H), 7.42 (d, J=8.5 Hz, 2H), 7.39-7.32 (m, 1H), 7.11 (d, J=7.1 Hz, 1H), 7.02-6.86 (m, 3H), 4.98 (dd, J=12.5, 5.5 Hz, 1H), 4.08 (s, 2H), 3.86-3.65 (m, 9H), 3.51 (m, 2H), 3.49-3.34 (m, 4H), 2.21 (m, 2H), 2.10-1.93 (m, 2H), 1.62 (m, 2H), 1.27 (d, J=6.9 Hz, 6H).

LC/MS (ESI) m/z 887 [M+H]⁺.

### Example 15: Synthesis of N-(2-(2-(2-(2-(4-(4-((5-chloro-4-((2-(isopropylsulfonyl)phenyl)amino)pyrimidin-2-yl)amino)phenyl)piperazin-1-yl)ethoxy)ethoxy)ethoxy)ethyl)-2-((2-(2,6-dioxopiperidin-3-yl)-1,3-dioxoisoindolin-4-yl)amino)acetamide (SK-3-87)

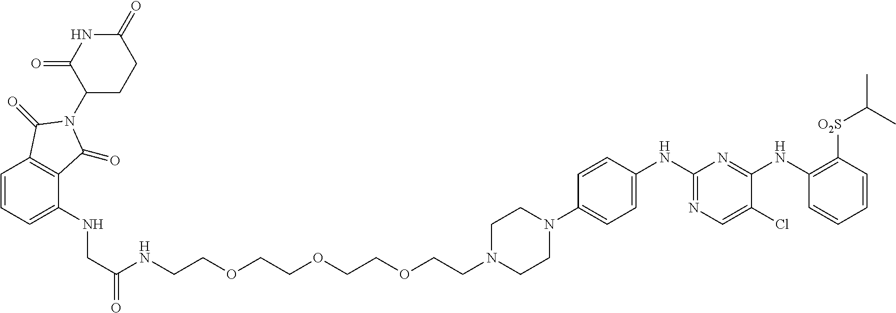 [SMILES: CC(C)S(=O)(=O)c1ccccc1Nc1nc(Nc2ccc(N3CCN(CCOCCOCCOCCNC(=O)CNc4cccc5c4C(=O)N(C4CCC(=O)NC4=O)C5=O)CC3)cc2)ncc1Cl]

### SK-3-87

SK-3-87 was prepared in an analogous manner to compound SK-3-91 in Example 11.

¹H NMR (400 MHz, Methanol-d4, TFA salt) 6 8.36 (d, J=8.4 Hz, 1H), 8.00 (s, 1H), 7.81 (dd, J=8.0, 1.6 Hz, 1H), 7.57 (ddd, J=8.7, 7.4, 1.6 Hz, 1H), 7.42 (dd, J=8.5, 7.1 Hz, 1H), 7.35-7.17 (m, 3H), 6.97 (d, J=7.1 Hz, 1H), 6.86 (d, J=8.5 Hz, 2H), 6.74 (d, J=8.5 Hz, 1H), 4.93 (dd, J=12.4, 5.4 Hz, 1H), 3.84 (m, 2H), 3.76 (dd, J=5.8, 4.1 Hz, 2H), 3.66-3.54 (m, 7H), 3.50 (m, 4H), 3.44 (m, 2H), 3.39-3.21 (m, 6H), 3.00 (m, 2H), 2.81-2.48 (m, 4H), 2.16-1.84 (m, 2H), 1.13 (d, J=6.8 Hz, 6H).

LC/MS (ESI) m/z: 975 [M+H]⁺.

### Example 16: Synthesis of N-(2-(2-(2-(4-(4-((5-chloro-4-((2-(isopropylsulfonyl)phenyl)amino)pyrimidin-2-yl)amino)phenyl)piperazin-1-yl)ethoxy)ethoxy)ethyl)-2-((2-(2,6-dioxopiperidin-3-yl)-1,3-dioxoisoindolin-5-yl)amino)acetamide (LT2-49)

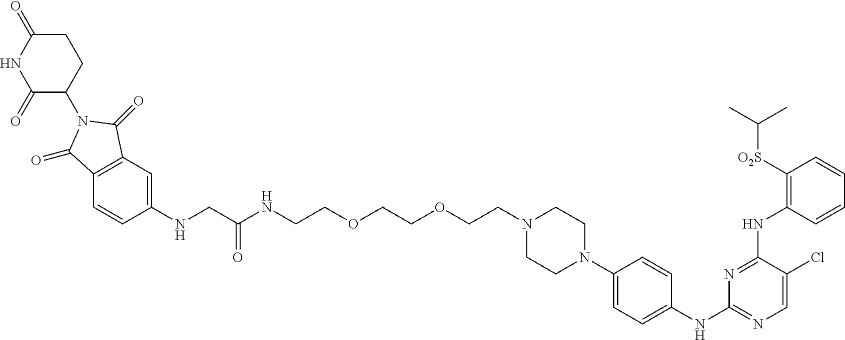 [SMILES: CC(C)S(=O)(=O)c1ccccc1Nc1nc(Nc2ccc(N3CCN(CCOCCOCCNC(=O)CNc4ccc5c(c4)C(=O)N(C4CCC(=O)NC4=O)C5=O)CC3)cc2)ncc1Cl]

### LT2-49

LT2-49 was prepared in an analogous manner to compound SK-3-91 in Example 11.

¹H NMR (400 MHz, DMSO-d6, TFA salt)S 11.07 (s, 1H), 9.70 (s, 1H), 9.51 (s, 1H), 9.39 (s, 1H), 8.63 (s, 1H), 8.25 (s, 1H), 8.12 (t, J=5.7 Hz, 1H), 7.85 (dd, J=8.0, 1.6 Hz, 1H), 7.75 (td, J=8.4, 7.9, 1.6 Hz, 1H), 7.59 (d, J=8.3 Hz, 1H), 7.48 (d, J=8.5 Hz, 2H), 7.37 (td, J=7.7, 1.2 Hz, 1H), 7.01-6.86 (m, 3H), 6.86 (dd, J=8.4, 2.2 Hz, 1H), 5.03 (dd, J=12.8, 5.4 Hz, 1H), 3.85 (s, 2H), 3.82-3.66 (m, 4H), 3.57 (dt, J=11.7, 6.1 Hz, 6H), 3.50-3.36 (m, 6H), 3.34-3.11 (m, 4H), 3.09-2.78 (m, 2H), 2.65-2.53 (m, 2H), 2.06-1.92 (m, 1H), 1.17 (d, J=6.8 Hz, 6H).

LC/MS (ESI) m/z: 931 [M+H]⁺.

### Example 17: Synthesis of N-(2-(2-(2-(4-(4-((5-chloro-4-((2-(isopropylsulfonyl)phenyl)amino)pyrimidin-2v1)amino)phenyl)piperazin-1-yl)ethoxy)ethyl)-2-((2-(2,6-dioxopiperidin-3-yl)-1,3-dioxoisoindolin-5-yl)amino)acetamide (SK-3-93)

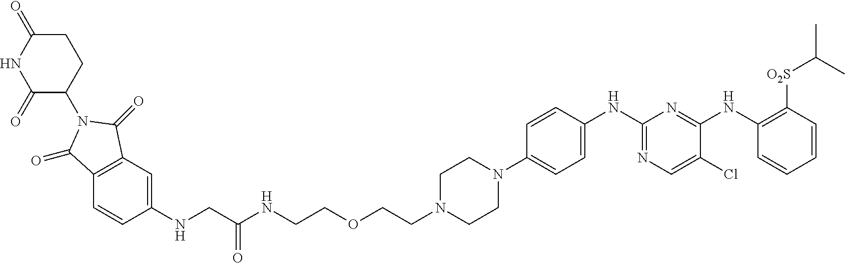 [SMILES: CC(C)S(=O)(=O)c1ccccc1Nc1nc(Nc2ccc(N3CCN(CCOCCNC(=O)CNc4ccc5c(c4)C(=O)N(C4CCC(=O)NC4=O)C5=O)CC3)cc2)ncc1Cl]

### SK-3-93

SK-3-93 was prepared in an analogous manner to compound SK-3-91 in Example 11.

¹H NMR (400 MHz, Methanol-d4, TFA salt) δ 8.42 (d, J=8.4 Hz, 1H), 8.04 (s, 1H), 7.81 (dd, J=8.0, 1.6 Hz, 1H), 7.57 (s, 1H), 7.47 (d, J=9.0 Hz, 1H), 7.36-7.21 (m, 3H), 6.93-6.72 (M, 4H), 4.93 (dd, J=12.4, 5.5 Hz, 1H), 3.86 (M, 2H), 3.71 (t, J=4.9 Hz, 2H), 3.50 (m, 6H), 3.38 (m, 2H), 3.32-3.24 (m, 4H), 2.80-2.59 (m, 4H), 2.17-2.02 (m, 1H), 1.94 (dd, J=8.2, 5.1 Hz, 2H), 1.15 (d, J=6.8 Hz, 6H).

LC/MS (ESI) m/z: 887 [M+H]⁺.

### Example 18: Synthesis of Synthesis of N-(2-(2-(2-(4-(4-((5-chloro-4-((2-(isopropylsulfonyl)phenyl)amino)pyrimidin-2-yl)amino)phenyl)piperazin-1-yl)ethoxy)ethoxy)ethoxy)ethyl)-2-((2-(2,6-dioxopiperidin-3-yl)-1,3-dioxoisoindolin-5-yl)amino)acetamide (SK-3-89)

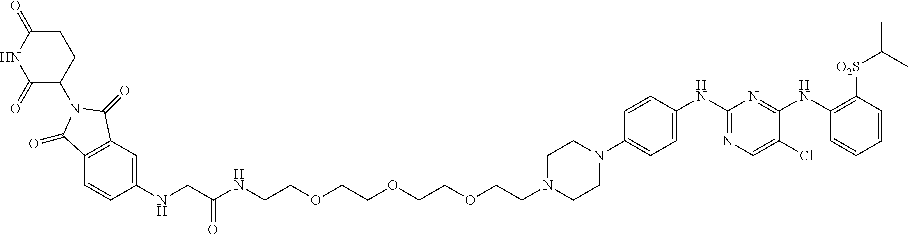 [SMILES: CC(C)S(=O)(=O)c1ccccc1Nc1nc(Nc2ccc(N3CCN(CCOCCOCCOCCNC(=O)CNc4ccc5c(c4)C(=O)N(C4CCC(=O)NC4=O)C5=O)CC3)cc2)ncc1Cl]

SK-3-89 was prepared in an analogous manner to compound SK-3-91 in Example 11.

¹H NMR (400 MHz, Methanol-d4, TFA salt) δ 8.34 (d, J=8.3 Hz, 1H), 8.03 (s, 1H), 7.82 (dd, J=8.0, 1.5 Hz, 1H), 7.63-7.54 (m, 1H), 7.47 (d, J=8.3 Hz, 1H), 7.31 (t, J=7.7 Hz, 1H), 7.27 (d, J=8.4 Hz, 2H), 6.96-6.79 (m, 3H), 6.74 (dd, J=8.3, 2.1 Hz, 1H), 4.92 (dd, J=12.4, 5.5 Hz, 1H), 3.79 (m, 2H), 3.76 (m, 2H), 3.70-3.51 (m, 10H), 3.47 (m, 8H), 3.45-3.25 (m, 2H), 2.75-2.58 (m, 2H), 2.03-1.89 (m, 1H), 1.13 (d, J=6.8 Hz, 6H).

LC/MS (ESI) m/z: 975 [M+H]⁺.

### Example 19: Synthesis of 3-(2-(2-(4-(4-((5-chloro-4-((2-(isopropylsulfonyl)phenyl)amino)pyrimidin-2-yl)amino)phenyl)piperazin-1-yl)ethoxy)ethoxy)-N-(2-((2-(2,6-dioxopiperidin-3-yl)-1-1722 oxoisoindolin-4-yl)amino)ethyl)propenamide (TL13-97)

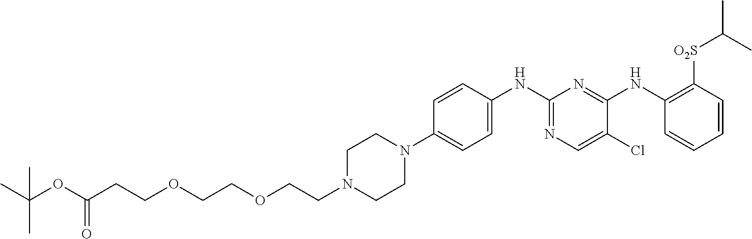 [SMILES: CC(C)S(=O)(=O)c1ccccc1Nc1nc(Nc2ccc(N3CCN(CCOCCOCCC(=O)OC(C)(C)C)CC3)cc2)ncc1Cl]

### tert-Butyl3-(2-(2-(4-(4-((5-chloro-4-((2-(isopropylsulfonyl)phenyl)amino)pyrimidin-2-1709 yl)amino)phenyl)piperazin-1-yl)ethoxy)ethoxy)propanoate (3)

Intermediate 1 (in scheme 6, below) was prepared according to literature (Huang et al., Cell Chem. Biol. 25:88-99 (2018)). To a solution of 1 (590 mg, 1.0 mmol) in DCM (18 mL) was added TFA (1.8 mL), and the mixture was stirred at RT for 2 hours. The reaction mixture was concentrated under reduced pressure, and resulting residue was redissolved in acetonitrile (5 mL) before the addition of commercially available intermediate 2 (355 mg, 1.2 mmol) and potassium carbonate (414 mg, 3.0 mmol). The mixture was stirred overnight at 80° C. The reaction was allowed to cool to RT and then diluted with DCM (50 mL). The resulting mixture was filtered, and the filtrate was concentrated under reduced pressure and purified by column chromatograph (dichloromethane:methanol=10:1) to obtain compound 3 as a colorless oil (495 mg, yield 70%).

LC/MS (ESI) m/z: 703 [M+H]⁺.

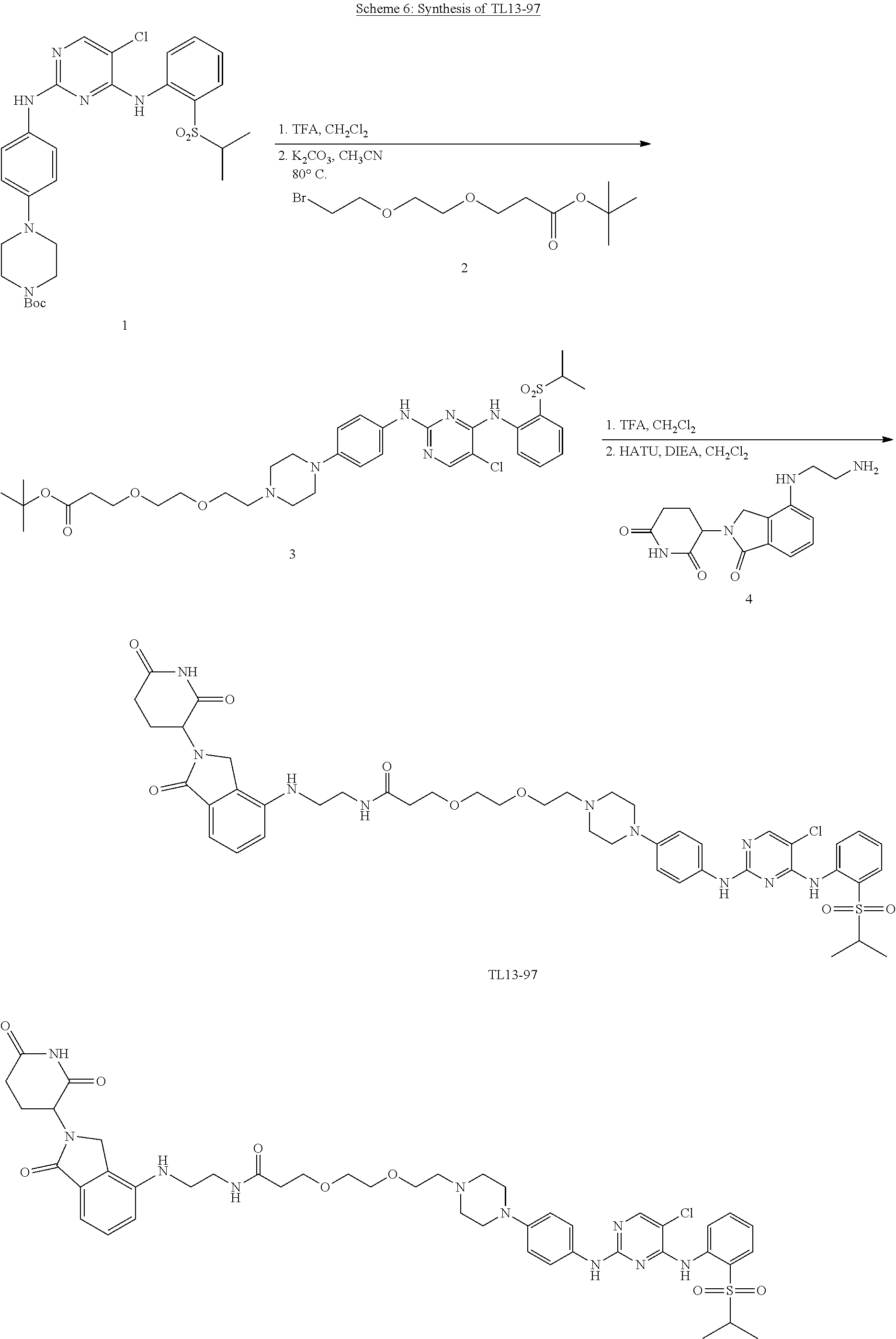 [SMILES: CC(C)S(=O)(=O)c1ccccc1Nc1nc(Nc2ccc(N3CCN(C(=O)OC(C)(C)C)CC3)cc2)ncc1Cl ; CC(C)(C)OC(=O)CCOCCOCCBr ; CC(C)S(=O)(=O)c1ccccc1Nc1nc(Nc2ccc(N3CCN(CCOCCOCCC(=O)OC(C)(C)C)CC3)cc2)ncc1Cl ; NCCNc1cccc2c1CN(C1CCC(=O)NC1=O)C2=O ; CC(C)S(=O)(=O)c1ccccc1Nc1nc(Nc2ccc(N3CCN(CCOCCOCCC(=O)NCCNc4cccc5c4CN(C4CCC(=O)NC4=O)C5=O)CC3)cc2)ncc1Cl ; CC(C)S(=O)(=O)c1ccccc1Nc1nc(Nc2ccc(N3CCN(CCOCCOCCC(=O)NCCNc4cccc5c4CN(C4CCC(=O)NC4=O)C5=O)CC3)cc2)ncc1Cl]

### TL13-97

TL13-97 was prepared in an analogous manner to compound SK-3-91 in Example 11 from intermediate 4, which was prepared as described in Zhou et al., Eur. J. Med. Chem. 187:111952 (2020).

¹H NMR (500 MHz, DMSO-d6) δ 11.01 (s, 1H), 9.69 (s, 1H), 9.52 (s, 1H), 9.41 (s, 1H), 8.64 (s, 1H), 8.26 (s, 1H), 8.06 (t, J=5.6 Hz, 1H), 7.85 (dd, J=8.0, 1.6 Hz, 1H), 7.80-7.68 (m, 1H), 7.49 (d, J=8.4 Hz, 2H), 7.42-7.34 (nm, 1H), 7.28 (t, J=7.7 Hz, 1H), 7.07-6.87 (nm, 3H), 6.82 (d, J=8.1 Hz, 1H), 5.12 (dd, J=13.3, 5.1 Hz, 1H), 4.29-4.04 (m, 2H), 3.83-3.70 (m, 4H), 3.64 (t, J=6.4 Hz, 2H), 3.61-3.51 (m, 6H), 3.49-3.41 (m, 1H), 3.37 (d, J=5.1 Hz, 2H), 3.30-3.14 (m, 6H), 3.03-2.88 (m, 3H), 2.66-2.57 (mi, 1H), 2.35 (t, J=6.4 Hz, 2H), 2.29 (dd, J=13.2, 4.6 Hz, 1H), 2.08-1.98 (m, 1H), 1.17 (d, J=6.8 Hz, 6H).

LC/MS (ESI)n/z: 931[M+H]⁺.

### Example 20: Synthesis of N-(3-(4-(6-amino-5-(1-oxo-1,2,3,4-tetrahydroisoquinolin-6-yl)pyridin-3-yl)-N-cyclopropylphenylsulfonamido)propyl)-1-((2-(2,6-dioxopiperidin-3-yl)-1,3-dioxoisoindolin-4-yl)amino)-3,6,9,12,15-pentaoxaoctadecan-18-amide (WH-9533-099) and N-(3-((4-(6-amino-5-(1-oxo-1,2,3,4-tetrahydroisoquinolin-6-yl)pyridin-3-yl)-N-cyclopropyl-3-fluorophenyl)sulfonamido)propyl)-1-((2-(2,6-dioxopiperidin-3-yl)-1,3-dioxoisoindolin-4-yl)amino)-3,6,9,12,15-pentaoxaoctadecan-18-amide. (WH-9533-153)

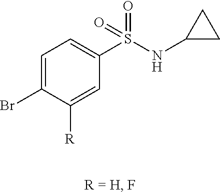

A Solution of 4-bromo-3-fluorobenzenesulfonyl chloride (0.55 g, 2.0 mmol, 1.0 eq) in DCM was added dropwise to a stirring solution of cyclopropanamine (0.15 g, 2.6 mmol, and 1.3 eq) and TEA (0.84 mL, 6.0 mmol, 3 eq) in DCM at 5-10° C. The resulting mixture was allowed warm to RT and stirred for 1 hour. The mixture was concentrated under reduced pressure, and the residue was purified by flash column chromatography (0% to 30% EtOAc in hexanes) to obtain above desired product, wherein R is F (0.54 g, 92%).

LC/MS (ESI) m/z: 293.98 [M+H]⁺: calcd for C₉H₁₀BrFNO₂S⁺: 294.14.

A mixture of 4-bromobenzene-1-sulfonyl chloride (2.56 g, 10.0 mmol, 1.0 eq), cyclopropanamine (0.58 g, 10.0 mmol, 1.0 eq), and Et₃N (1.17 g, 11.0 mmol, 1.1 eq) in DCM (20 mL) was stirred at RT for 22 hours. The mixture was concentrated under reduced pressure, and the residue was purified by flash column chromatography (silica gel, PE:EtOAc=4:1) to obtain above desired compound, wherein R is H, as a white solid (2.24 g, 81%).

LC/MS (ESI) m/z 277.9 (isotope) [M+H]⁺; calcd for C9H₁₁BrNO₂S⁺: 275.97.

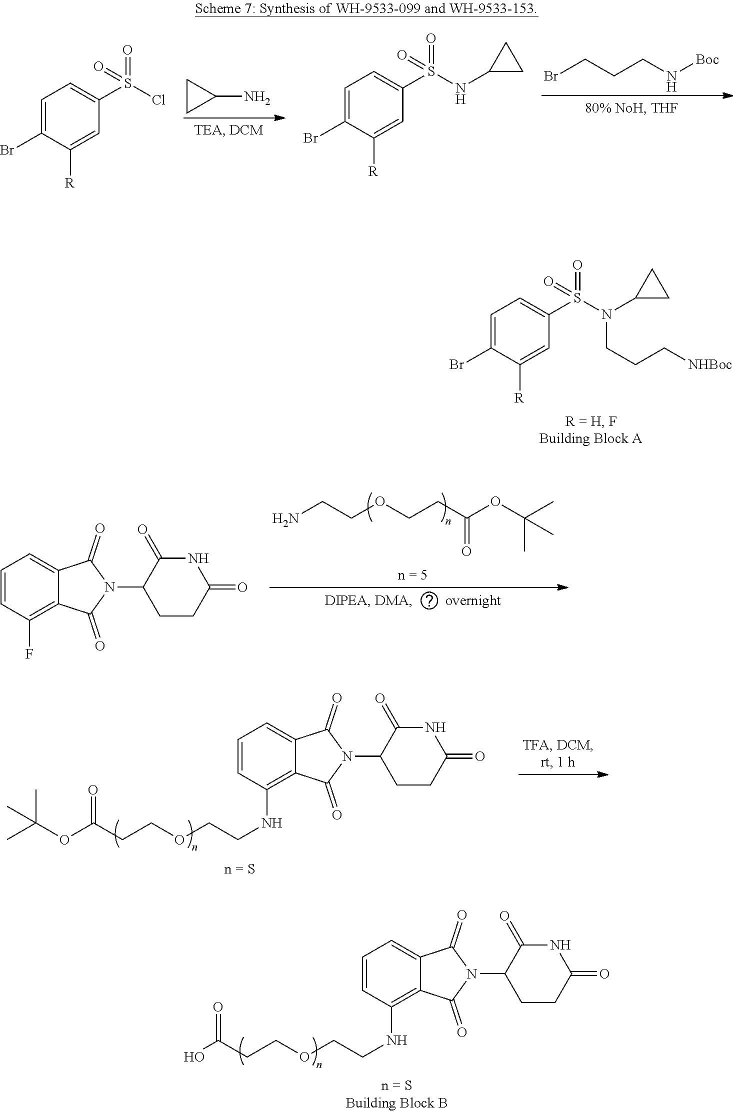

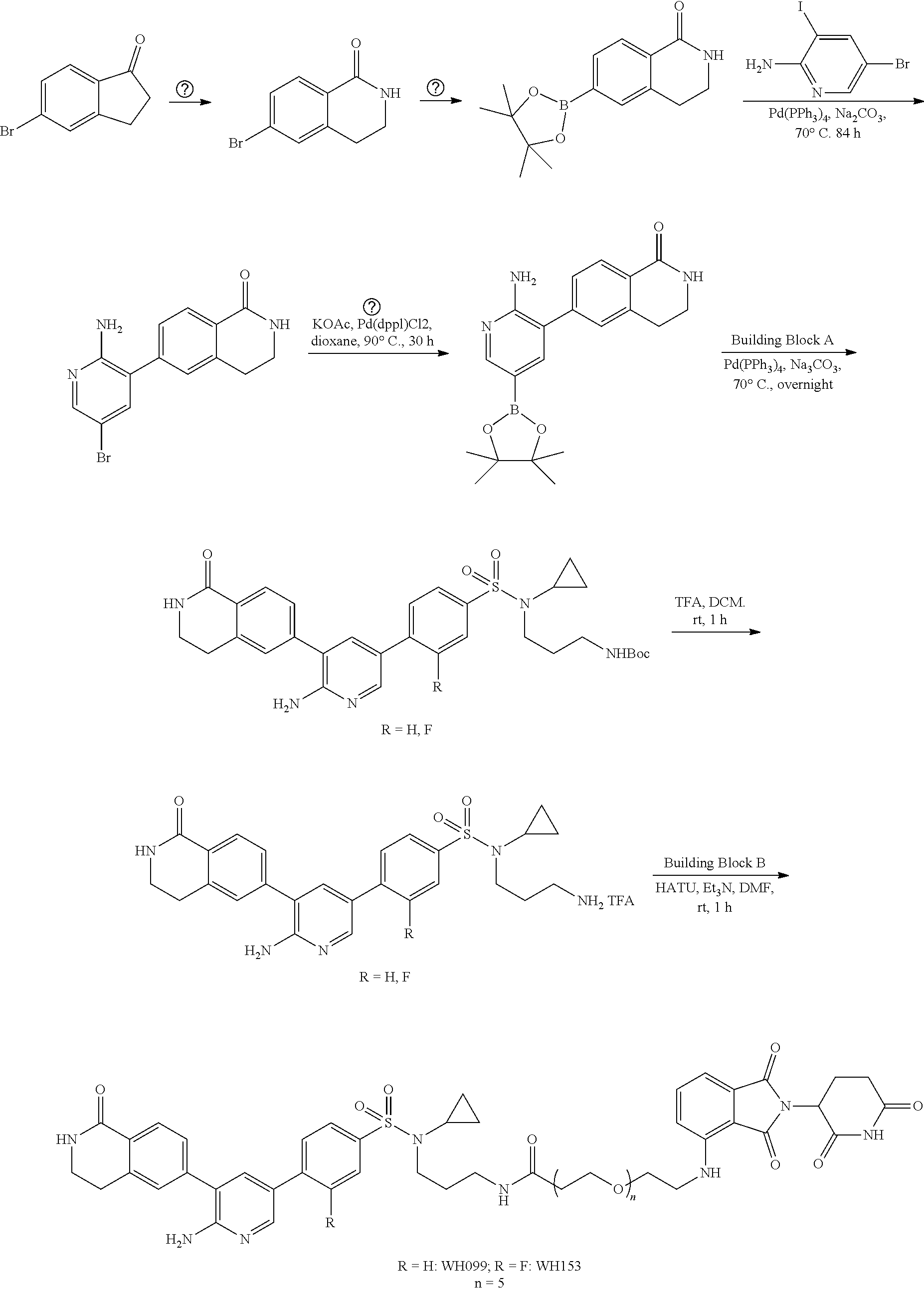

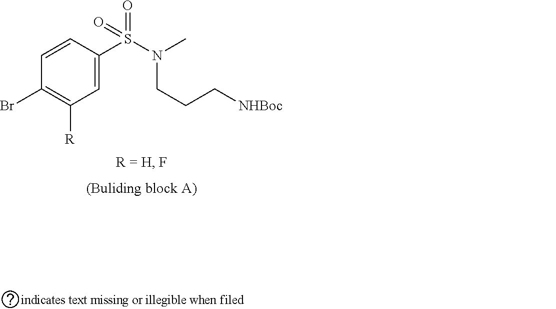

To a stirring suspension of 60% NaH in anhydrous THF was added dropwise a solution of 4-bromo-N-cyclopropyl-3-fluorobenzenesulfonamide (0.50 g, 1.7 mmol) in anhydrous THF (5 mL) at 5-10° C. under nitrogen. The resulting mixture was allowed to warm to RT and stirred for 20 min before the addition of a solution of tert-butyl (3-bromopropyl)carbamate (0.61 g, 2.5 1758 mmol) in anhydrous THF. The reaction mixture was heated overnight at 55° C. The crude mixture was quenched with saturated aqueous NH₄Cl and extracted with EtOAc. The combined organic layers were washed with water and brine, dried over anhydrous MgSO₄, filtered and concentrated under reduced pressure. The resulting residue was purified by flash column chromatography (0% to 30% EtOAc in hexanes) to obtain above desired product, wherein R is F (0.44 g, yield 58%).

LC/MS (ESI) m/z: 351.07 [M−99]⁺; calcd for C₁₇H25BrFN₂O4S⁺: 451.35.

A mixture of 4-bromo-N-cyclopropylbenzenesulfonamide (1.91 g, 7.0 mmol, 1.0 eq), tert-butyl (3-bromopropyl)carbamate (1.67 g, 14.0 mmol, 2.0 eq) and K₂CO₃ (4.80 g, 35.0 mmol, 5.0 eq) in DMF (70 mL) was stirred at RT for 17 hours. The reaction was quenched with water (200 mL) and extracted with EtOAc (200 mL×3). The combined organic layers were washed with brine (250 mL×3), dried over anhydrous Na₂SO₄, filtered, concentrated under reduced pressure, and purified by flash column chromatography (silica gel, PE:EtOAc=9:1) to obtain above desired compound, wherein R is H, as a white solid (3.12 g, quantitative yield).

LC/MS (ESI) m/z: 332.9 [M−99]+, 454.8 [M+Na]⁺; calcd for C₁₇H₂₆BrN₂O₄S⁺: 433.08.

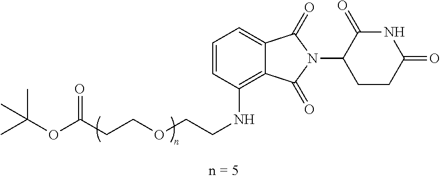

A mixture of 2,6-dioxopiperidin-3-yl)-4-fluoroisoindoline-1,3-dione (0.28 g, 1.0 mmol), amino-PEG-tert-butyl ester [0.36 g (PEG5), 1.0 mmol], and DIPEA (0.24 mL, 1777 1.5 mmol) in 3 mL dimethylacetamide (DMA) was heated at 90° C. in sealed reaction tube overnight. The reaction was cooled to RT, and the crude was directly subjected to preparative HPLC purification (MeCN/H₂O v/v 0.5% TFA). Isolated product was further purified using flash column chromatography (80% to 100% EtOAc in hexanes) to obtain the above desired compound as a yellow oil (0.28 g, yield 44%).

LC/MS (ESI) m/z: 622.23 [M+H]+; calcd for C₃₀H₄₄N₃O₁₁⁺: 622.30.

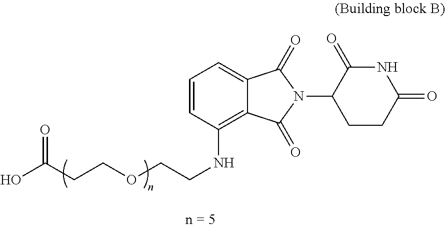

To a solution of the PEG5-isoindoline-1,3-dione tert-butyl ester product o(0.21 g, 0.3 mmol) in DCM (8 mL) was added TFA (4 mL). The mixture was stirred at RT for 1 hour. The mixture was concentrated under reduced pressure, and the crude was purified by preparative HPLC (C18 column, CH₃CN/H₂O, neutral condition) to obtain building block B (0.13 g, yield 68%).

LC/MS (ESI) m/z: 566.32 [M+H]⁺; calcd for C₂₆H₃₆N₃O₁₁⁺: 566.23.

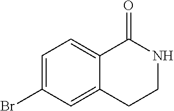 [SMILES: O=C1NCCc2cc(Br)ccc21]

### 6-Bromo-3,4-dihydroisoquinolin-1(2H)-one

To a stirred solution of 5-bromo-2,3-dihydro-1H-inden-1-one (10.00 g, 1792 47.4 mmol) and methanesulfonic acid (45.50 g, 473.9 mmol) in DCM (75 mL) was slowly added NaN₃ (6.20 g, 94.8 mmol)(in portions) at −5˜0° C. under N₂ atmosphere. The reaction was stirred at 0° C. for 3 hours. pH of reaction mixture was adjusted to 10 with 20% aqueous NaOH, and resulting mixture was extracted with DCM. The combined organic layers were washed with water (3×) and then with brine, dried over anhydrous 1797 MgSO₄, filtered and concentrated. The residue was purified by flash column chromatography (0% to 70% EtOAc in hexanes) to obtain above desired product (6.9 g, yield 64%).

LC/MS (ESI) m/z: 226.08 [M+H]⁺; calcd for C₉H₉BrNO⁺: 226.07.

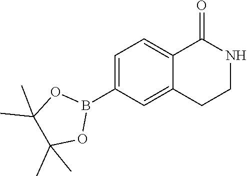 [SMILES: CC1(C)OB(c2ccc3c(c2)CCNC3=O)OC1(C)C]

### 6-(4,4,5,5-Tetramethyl-1,3,2-dioxaborolan-2-yl)-3,4-dihydroisoquinolin-1(2H)-one

A mixture of 6-bromo-3,4-dihydroisoquinolin-1(2H)-one (3.40 g, 15.0 mmol), bis(pinacolato)diboron (5.73 g, 22.5 mmol), potassium acetate (2.95 g, 30.0 mmol), and Pd(dppf)Cl₂ (1.10 g, 1.5 mmol) in dioxane (75 mL) was heated at 85° C. for 20 hours under N₂. The mixture was concentrated, and the residue was purified by flash column chromatography (0% to 80% EtOAc in hexanes) to obtain above desired product (3.4 g, yield 83%).

LC/MS (ESI) m/z: 274.28 [M+H]⁺; calcd for C₁₅H₂₁BNO₃⁺: 274.16.

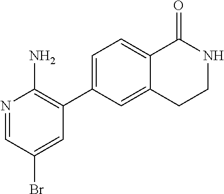 [SMILES: Nc1ncc(Br)cc1-c1ccc2c(c1)CCNC2=O]

### 6-(2-Amino-5-bromopyridin-3-yl)-3,4-dihydroisoquinolin-1(2H)-one

A mixture of 6-(4,4,5,5-tetramethyl-1,3,2-dioxaborolan-2-yl)-3,4-dihydroisoquinolin-1(2H)-one (3.40 g, 12.5 mmol), 5-bromo-3-iodopyridin-2-amine (4.50 1810 g, 14.9 mmol), sodium carbonate (2.64 g, 24.9 mmol), and Pd(PPh3)4 (1.44 g, 1.3 mmol) in 1,4-dioxane (80 mL) and water (10 mL) was heated at 70° C. for 64 hours under N₂ atmosphere. The mixture was concentrated under reduced pressure, and the residue was purified by flash column chromatography (0% to 25% MeOH in DCM) to obtain above desired product (2.1 g, yield 53%).

LC/MS (ESI) m/z: 318.18 [M+H]⁺; calcd for C₁₄H₁₃BrN₃O⁺: 318.17.

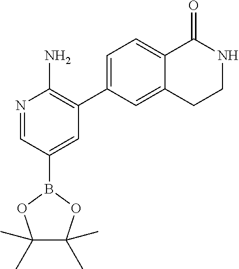 [SMILES: CC1(C)OB(c2cnc(N)c(-c3ccc4c(c3)CCNC4=O)c2)OC1(C)C]

A mixture of 6-(2-amino-5-bromopyridin-3-yl)-3,4-dihydroisoquinolin-1(2H)-one (1.00 g, 3.1 mmol), Bis(pinacolato)diboron (1.20 g, 4.7 mmol), potassium acetate (0.62 g, 6.3 mmol), and Pd(dppf)Cl₂(0.23 g, 0.3 mmol) in 1,4-dioxane (30 mL) was heated at 90° C. for 20 hours under N₂ atmosphere. The mixture was concentrated reduced pressure. The resulting residue was dissolved in DCM and washed with water (2×) and brine, dried over MgSO₄, filtered, and concentrated under reduced pressure to afford 2 g crude product, which was used in next step without further purification.

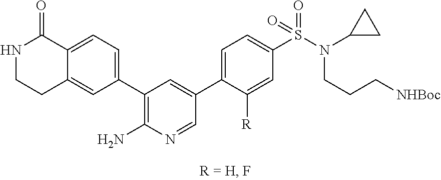

A mixture of tert-butyl (3-((4-bromo-N-cyclopropyl-3-fluorophenyl)sulfonamido)propyl)carbamate (0.25 g, 0.56 mmol, 1.0 eq.), 6-(2-amino-5-(4,4,5,5-tetramethyl-1,3,2-dioxaborolan-2-yl)pyridin-3-yl)-3,4-dihydroisoquinolin-1(2H)-one (0.34 g, 0.56 mmol, 1.0 eq.), sodium carbonate (0.12 g, 1.12 mmol, 2.0 eq.), and Pd(PPh3)4 (0.065 g, 0.056 mmol, 0.1 eq.) in 1,4-dioxane and water (12 mL, v/v=5:1) was 1830 heated at 90° C. overnight under N₂ atmosphere. The mixture was concentrated under reduced pressure, and the residue was purified by flash column chromatography to afford desire product, wherein R is F (18 mg, yield 5.2%).

LCMS (ESI) m/z: 1832 610.39 [M+H]⁺; calcd for C₃₁H₃₂FN₅O₅S³⁰: 610.25.

A mixture of (6-amino-5-(1-oxo-1,2,3,4-tetrahydroisoquinolin-6-yl)pyridin-1834 3-yl)boronic acid (0.5 g, 1.94 mmol, 1.0 eq), tert-butyl (3-(4-bromo-N-1835 cyclopropylphenylsulfonamido)propyl)carbamate (0.86 g, 1.94 mmol, 1.0 eq), Pd(PPh3)2C12 (0.27 g, 0.39 mmol, 0.2 eq), and K₂CO3 (1.08 g, 7.76 mmol, 4.0 eq) in DMF (25 mL) was stirred at I 10C for 4.5 hours. After 41% of target compound was observed by LC-MS, the reaction mixture was allowed to cool to RT, diluted with water (30 mL) and extracted with EtOAc (60 mL-3). The combined organic layers were washed with brine (100 mL), dried over anhydrous Na₂SO₄, and concentrated under reduced pressure. The resulting residue was purified by flash column chromatography (silica gel, PE:EtOAc=9:1 to MeOH:EtOAc=2:8) to give the desired compound (200 mg) as a yellow solid with only 80% purity. The isolated product was further purified by preparative HPLC (C18 column. CH₃CN/H₂O/HCOOH (0.01%)) to obtain pure desired compound, wherein R is H, as a white solid (0.17 g, yield 15%).

LCMS (ESI) m/z: 592.0 [M+H]⁺; calcd for C₃₁H₃₈N₅O₅S⁺: 592.26.

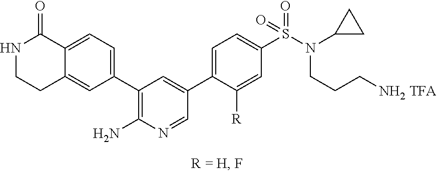

To a solution of a Boc-protected product from above step in DCM was added TFA 2 mL, and the mixture was stirred at room temperature for 30 minutes. The resulting mixture was concentrated under reduced pressure to obtain the respective TFA salt, which was used directly in next step.

When R=F: LCMS (ESI) m/z: 510.28 [[M+H]⁺; calcd for C₂₆H₂₈FN₅O₃S⁺: 509.60.

When R=H: LCMS (ESI) m/z: 492.28 [M+H]⁺; calcd for C₂₆H₃₀N₅O₃S⁺: 492.21.

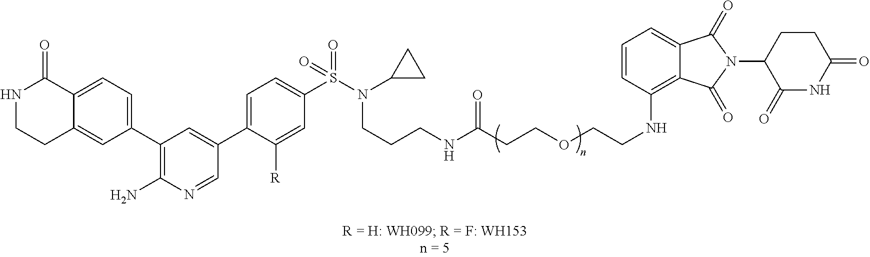

To a mixture of a TFA salt from above step compound (R=F: 0.02 g, 0.03 mmol, 1.0 eq. R=H: 0.05 g, 0.08 mmol, 1.0 eq.) and building block B compound (0.02 g, 0.03 mmol, 1.0 eq) in DMSO was added HATU (1.5 eq) and Et3N (4.0 eq.). The reaction was stirred at room temperature for 1 hour. The crude mixture was purified by preparative HPLC (C18 column, CH₃/CN/H₂O, neutral condition or with 0.05% TFA) and normal phase flash chromatography [0% to 20% MeOH in EtOAc/DCM (v/v=1:1)] to afford the desired product: WH-9533-153 (14 mg, 45%) or WH-9533-099 (47 mg, 51%).

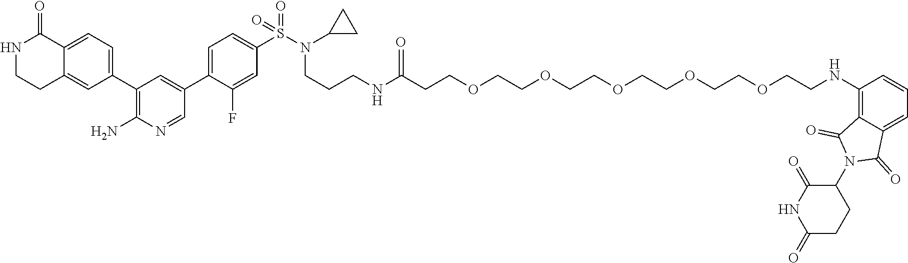 [SMILES: Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2F)cc1-c1ccc2c(c1)CCNC2=O]

### WH-9533-153

¹H NMR (500 MHz, CDCl₃) δ 10.58 (s, 1H), 8.36 (s, 1H), 8.18 (d, J=7.9 Hz, 1H), 7.75-7.55 (m, 2H), 7.49 (t, J=7.7 Hz, 1H), 7.37 (s, 1H), 7.10 (d, J=7.1 Hz, 1H), 7.00 (t, J=5.7 Hz, 1H), 6.93 (d, J=8.6 Hz, 1H), 6.51 (dd, J=12.3, 6.7 Hz, 1H), 5.34 (s, 1H), 4.94 (dd, J=12.1, 5.3 Hz, 1H), 3.72 (dt, J=17.4, 8.9 Hz, 3H), 3.69-3.57 (m, 9H), 3.54-3.42 (m, 2H), 3.35-3.22 (m, 2H), 3.07 (t, J=6.5 Hz, 1H), 2.94-2.68 (m, 2H), 2.49 (t, J=5.7 Hz, 1H), 2.22-2.01 (m, 1H), 1.91-1.77 (m, 1H), 0.88 (t, J=7.7 Hz, 1H), 0.73 (q, J=6.1 Hz, 1H).

¹³C NMR (126 MHz, CDCl₃) 172.46, 171.84, 169.36, 169.27, 167.72, 165.73, 160.16, 158.15, 155.81, 146.83, 141.05, 140.02, 138.41, 136.05, 132.52, 130.35, 129.07, 128.72, 127.59, 127.33, 123.74, 120.45, 116.83, 115.85, 115.64, 111.61, 110.29, 70.64, 70.48, 70.43, 70.36, 70.25, 70.10, 69.41, 67.35, 48.95, 48.80, 42.36, 42.26, 40.15, 40.01, 36.89, 36.40, 36.27, 31.56, 30.72, 28.46, 28.37, 22.81, 7.11.

LC/MS (ES) m/z: 1057.54 [M+H]⁺; calcd for C₅₂H₆₂FN₈O₁₃S⁺: 1057.41.

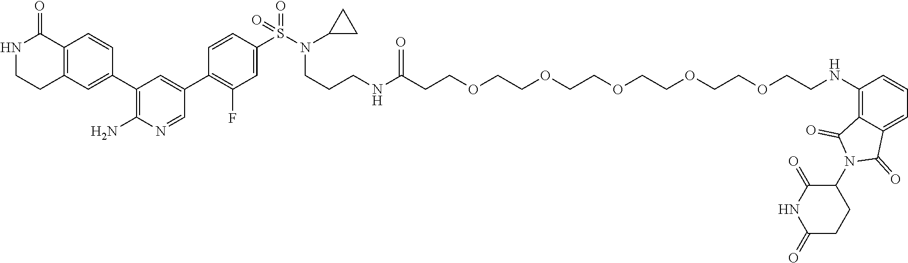 [SMILES: Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2F)cc1-c1ccc2c(c1)CCNC2=O]

### WH-9533-153

¹H NMR (500 MHz, DMSO-d6) δ 11.19 (s, 1H), 8.55 (s, 1H), 8.13-8.01 (m, 5H), 7.94-7.91 (m, 3H), 7.71-7.55 (m, 3H), 7.21 (d, J=8.6 Hz, 1H), 7.12 (d, J=7.0 Hz, 1H), 6.68 (t, J=5.8 Hz, 1H), 5.14 (dd, J=12.8, 5.5 Hz, 1H), 3.75-3.46 (m, 22H), 3.23 (t, J=7.6 Hz, 2H), 3.11 (q, J=6.7 Hz, 2H), 3.06 (t, J=6.6 Hz, 2H), 2.97 (ddd, J=17.8, 13.7, 5.5 Hz, 1H), 2.73-2.52 (m, 4H), 2.38 (t, J=6.4 Hz, 2H), 2.11 (dq, J=7.1, 4.3 Hz, 2H), 1.73 (p, J=7.1 Hz, 2H), 0.89-0.68 (m, 4H).

¹³C NMR (126 MHz, DMSO) δ 173.29, 170.54, 170.49, 169.40, 167.77, 164.70, 155.59, 146.87, 142.73, 141.40, 140.48, 140.04, 138.39, 136.69, 136.49, 132.55, 129.39, 128.41, 128.36, 128.21, 127.52, 126.77, 123.72, 122.01, 117.90, 111.15, 109.71, 70.29, 70.24, 70.12, 69.98, 69.35, 67.30, 49.03, 48.96, 42.17, 36.68, 36.66, 31.45, 30.74, 28.69, 28.29, 22.62, 7.32.

LC/MS (ESI) m/z: 1038.64 [M+H]⁺; calcd for C₅₂H₆₅N₈O₁₃S⁺: 1039.42.

### Example 21: Synthesis of N-(3-(2-((4-(4-(5-((2-(2,6-Dioxopiperidin-3-yl)-1,3-dioxoisoindolin-4-yl)oxy)pentyl)piperazin-1-yl)phenyl)amino)thieno[3,2-d]pyrimidin-7-yl)phenyl)methanesulfonamide (DB0614)

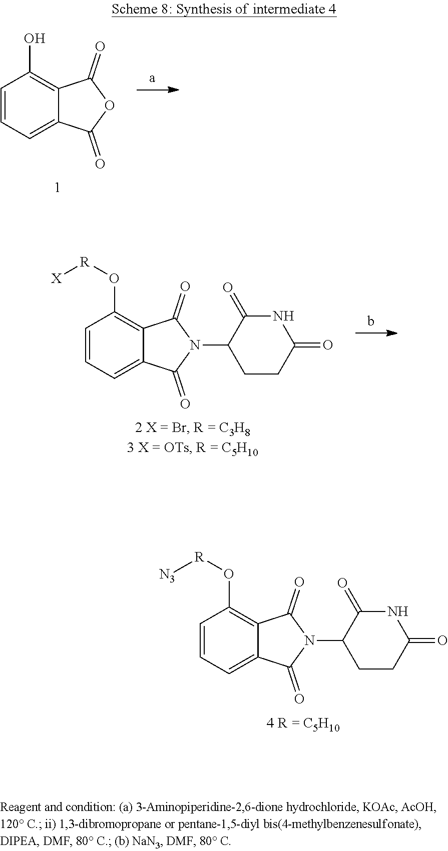

### General Procedure a for the Synthesis of Compound (2-3)

To a solution of compound 1 (20 g, 122 mmol) in AcOH (400 mL) was added 3-aminopiperidine-2,6-dione hydrochloride (24 g, 146 mmol), KOAc (36 g, 366 mmol) at RT. The reaction mixture was then stirred for 24 hours at 120° C., concentrated under reduced pressure. The residue was solidified by swirling in H₂O and filtered out to give black solid, which was used in the next reaction without any further purification.

To a solution of the isolated black solid (1.0 eq) in DMF (5.0 mL) was added 1,3-dibromopropane or pentane-1,5-diyl bis(4-methylbenzenesulfonate) (3.0 eq), DIPEA (3.0 eq) at RT. The reaction mixture was then stirred for 30 minutes at 60° C., quenched with water, and diluted with EtOAc. The organic layer was washed with brine, dried over MgSO₄, filtered, and concentrated under reduced pressure. The residue was purified by flash column chromatography (silica gel, 30-40% THF/hexane) to give compound 2-3 (yield 79-83%).

### 4-(3-Bromopropoxy)-2-(2,6-dioxopiperidin-3-yl)isoindoline-1,3-dione (2)

Black solid (498 mg, 1.59 mmol) was converted to the target compound using general procedure A. The residue was purified by flash column chromatography on silica gel (30-40% THF/hexane) to give compound 2 (498 mg, yield 79%).

¹H NMR (400 MHz, DMSO-d₆) δ 11.12 (s, 1H), 7.84 (t, J=7.8 Hz, 1H), 7.58 (d, J=8.8 Hz, 1H), 7.47 (d, J=8.6 Hz, 1H), 5.10 (dd, J=12.7, 5.1 Hz, 1H), 4.33 (t, J=5.6 Hz, 2H), 3.82-3.66 (m, 2H), 2.98-2.80 (m, 1H), 2.67-2.52 (m, 2H), 2.37-2.21 (m, 2H), 2.16-1.96 (m, 1H).

LRMS (ESI) m/z: 395 [M+H]⁺.

### 5-((2-(2,6-Dioxopiperidin-3-yl)-1,3-dioxoisoindolin-4-yl)oxy)pentyl 4-methylbenzenesulfonate (3)

Black solid (527 mg, 1.69 mmol) was converted to the target compound using general procedure A. The residue was purified by flash column chromatography (silica gel, 30-40% THF/hexane) to give compound 3 (721 mg, 83%).

LRMS (ESI) m/z: 515 [M+H]⁺.

### 4-((5-Azidopentyl)oxy)-2-(2,6-dioxopiperidin-3-yl)isoindoline-1,3-dione (4)

To a solution of compound 3 (319 mg, 0.62 mmol) in DMF (5.0 mL) was added NaN₃ (201 mg, 3.10 mmol) at RT. The reaction mixture was then stirred for 1 hour at 60° C., quenched with water and diluted with EtOAc. The organic layer was washed with brine, dried over MgSO₄, filtered, and concentrated under reduced pressure. The residue was purified by flash column chromatography (silica gel, 30-40% THF/hexane) to give compound 4 (212 mg, yield 89%).

LRMS (ESI) m/z: 386 [M+H]⁺.

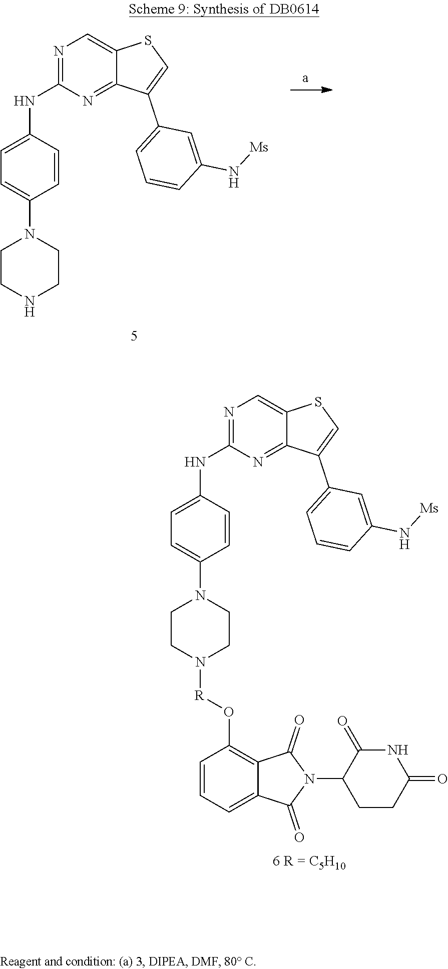

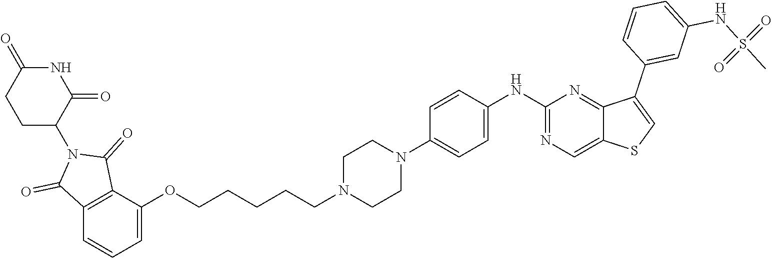 [SMILES: CS(=O)(=O)Nc1cccc(-c2csc3cnc(Nc4ccc(N5CCN(CCCCCOc6cccc7c6C(=O)N(C6CCC(=O)NC6=O)C7=O)CC5)cc4)nc23)c1]

### DB0614

To a solution of compound 5 (100 mg, 0.21 mmol) in DMF (2.0 mL) was added compound 3 (321 mg, 0.62 mmol) and DIPEA (0.11 mL, 0.62 mmol) at RT. The reaction mixture was then stirred for 30 minutes at 60° C., quenched with water, and diluted with EtOAc. The organic layer was washed with brine, dried over MgSO₄, filtered, and concentrated under reduced pressure. The residue was purified by flash column chromatography (silica gel, 30-40% THF/hexane) to give compound 6 (156 mg, yield 91%).

¹H NMR (4(0) MHz, DMSO-d6) δ 11.12 (s, 1H), 9.85 (s, 1H), 9.47 (s, 1H), 9.18 (s, 1H), 8.48 (s, 1H), 7.89-7.78 (m, 3H), 7.72 (d, J=8.6 Hz, 2H), 7.59-7.42 (m, 3H), 7.28 (d, J=8.0 Hz, 1H), 6.93 (d, J=8.7 Hz, 2H), 5.09 (dd, J=12.9, 5.4 Hz, 1H), 4.24 (t, J=6.2 Hz, 2H), 3.37-3.30 (m, 8H), 3.14-3.01 (m, 5H), 2.64-2.54 (m, 3H), 2.08-1.96 (m, 1H), 1.87-1.76 (m, 2H), 1.70-1.45 (m, 4H).

LRMS (ESI) m/z: 823 [M+H]⁺.

### Example 22: Synthesis of(2S,4R)-1-((S)-3,3-Dimethyl-2-(6-(4-(4-((7-(3-(methylsulfonamido)phenyl) thieno[3,2-d]pyrimidin-2-yl)amino)phenyl)piperazin-1-yl)hexanamido)butanoyl)-4-hydroxy-N—((S)-1-(4-(4-methylthiazol-5yl)phenyl)ethyl)pyrrolidine-2-carboxamide (RSS0680, 8)

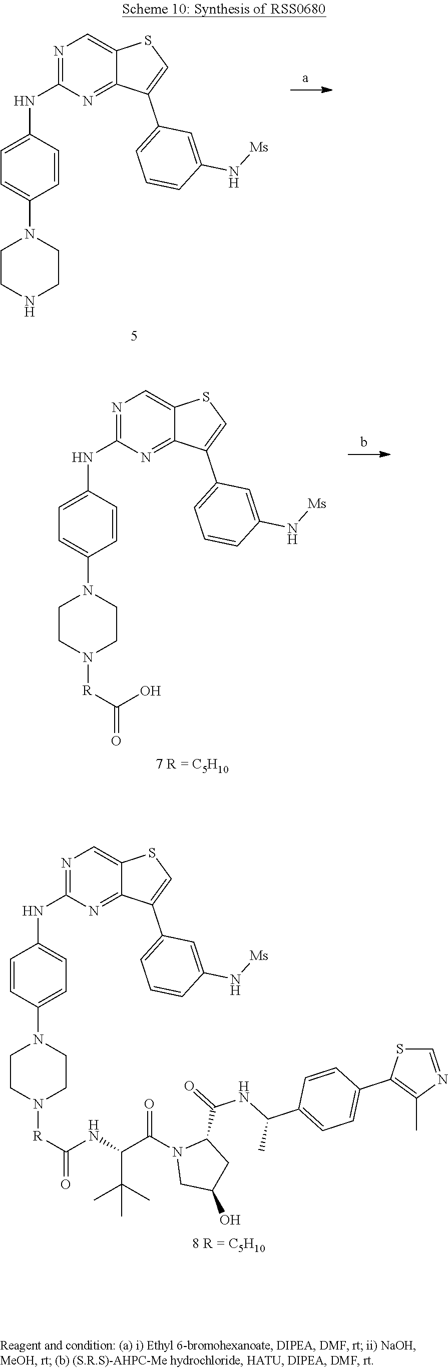

### 6-(4-(4-((7-(3-(Methylsulfonamido)phenyl)thieno[3,2-d]pyrimidin-2-yl)amino)phenyl)piperazin-1-yl)hexanoic acid (7)

To a solution of compound 5 (309 mg, 0.64 mmol) in DMF (5.0 mL) was added ethyl 6-bromohexanoate (430 mg, 1.93 mmol) and DIPEA (0.34 mL, 1.93 mmol) at RT. The reaction mixture was then stirred for 30 minutes at 60° C., quenched with water and diluted with EtOAc. The organic layer was washed with brine, dried over MgSO₄, filtered, and concentrated under reduced pressure. To a solution of crude intermediate ester in MeOH (10.0 mL) was added aqueous NaOH (129 mg, 3.21 mmol) at 0° C. The reaction mixture was then stirred for 1 hour at RT, diluted with EtOAc, and neutralized with aqueous citric acid. The organic layer was washed with brine, dried over MgSO₄, filtered, and concentrated under reduced pressure. The resulting residue was solidified by swirling in CH₂Cl₂/Et₂O and concentrated under reduced pressure to give compound 7, which was used in the next reaction without any further purification.

LRMS (ESI) m/z: 595 [M+H]⁺.

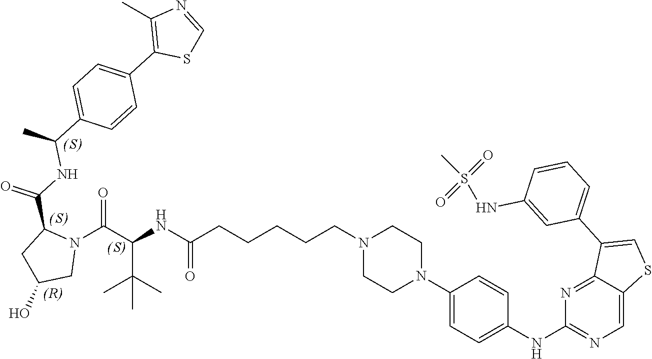

To a solution of compound 7 (116 mg, 0.20 mmol) in DMF (10 mL) was added (S,R,S)-AHPC-Me hydrochloride (104 mg, 0.23 mmol), HATU (222 mg, 0.59 mmol), and DIPEA (0.17 mL, 0.98 mmol) at 0° C. The reaction mixture was then stirred for 30 minutes at 0° C., quenched with water and diluted with EtOAc. The organic layer was washed with brine, dried over MgSO₄, filtered, and concentrated under reduced pressure. The residue was purified by flash column chromatography (silica gel, 20% EtOAc/hexane) to give compound 8 (121 mg, yield 61%).

¹H NMR (4(0) MHz, Acetone-d6) δ 9.08 (s, 1H), 8.83 (s, 1H), 8.69 (s, 1H), 8.56 (s, 1H), 8.36 (s, 1H), 8.08 (s, 1H), 7.91 (d, J=7.6 Hz, 1H), 7.80 (d, J=8.8 Hz, 2H), 7.75-7.67 (m, 1H), 7.53-7.36 (m, 6H), 7.13-7.07 (m, 11H), 6.99 (d, J=8.5 Hz, 2H), 5.08-4.98 (m, 1H), 4.69-4.56 (m, 2H), 4.47 (brs, 1H), 3.85 (s, 1H), 3.70 (d, J=11.1 Hz, 1H), 3.27-3.12 (m, 4H), 3.02 (s, 3H), 2.95-2.69 (m, 9H), 2.46 (s, 3H), 2.36-2.20 (m, 2H), 1.68-1.52 (m, 4H), 1.44 (d, J=6.9 Hz, 3H), 1.41-1.32 (m, 2H), 1.02 (s, 9H).

LRMS (ESI) m/z: 1021 [M+H]⁺.

### Example 23: Synthesis of N-(3-(7-((6-(4-(3-((2-(2,6-Dioxopiperidin-3-yl)-1,3-dioxoisoindolin-4-yl)oxy)propyl)piperazin-1-ylpyridin-3-yl)amino)-1-methyl-2-oxo-1,4-dihydropyrimido[4,5-d]pyrimidin-3(2H)-yl)-4-methylphenyl)-3-(trifluoromethyl)benzamide (DB0662, 10)

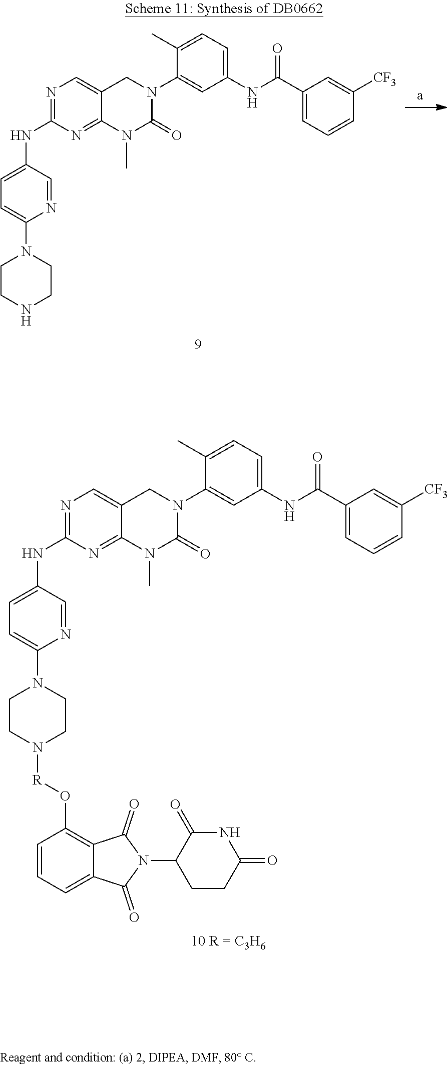

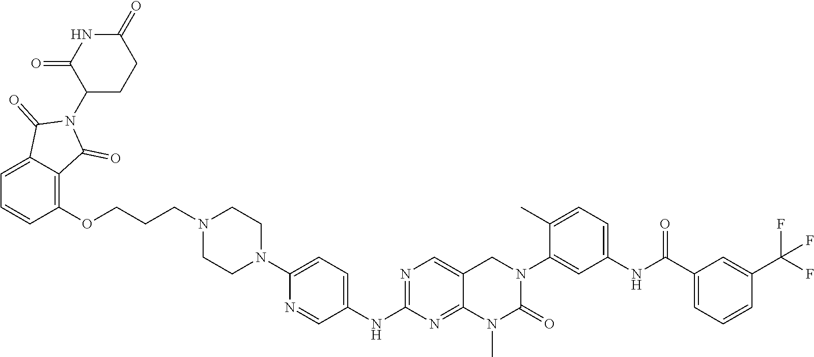 [SMILES: Cc1ccc(NC(=O)c2cccc(C(F)(F)F)c2)cc1N1Cc2cnc(Nc3ccc(N4CCN(CCCOc5cccc6c5C(=O)N(C5CCC(=O)NC5=O)C6=O)CC4)nc3)nc2N(C)C1=O]

To a solution of compound 9 (108 mg, 0.17 mmol) in DMF (3.0 mL) was added compound 2 (207 mg, 0.52 mmol) and DIPEA (0.09 mL, 0.52 mmol) at RT. The reaction mixture was then stirred for 30 1999 minutes at 60° C., quenched with water and diluted with EtOAc. The organic layer was washed 2000 with brine, dried over MgSO₄, filtered and concentrated under reduced pressure. The residue was purified by flash column chromatography on silica gel (30-40% THF/hexane) to give compound 10 (DB0662) (143 mg, 88%).

¹H NMR (400 MHz, Acetone-d₆) δ 9.96 (s, 1H), 9.83 (s, 1H), 8.52 (s, 1H), 8.4 (s, 1H), 8.31 (s, 1H), 8.30 (d, J=8.8 Hz, 1H), 8.07 (s, 1H), 8.03 (d, J=10.0 Hz, 1H), 7.92 (d, J=8.0 Hz, 1H), 7.87 (s, 1H), 7.82-7.75 (m, 2H), 7.66 (d, J=8.0 Hz, 1H), 7.51 (d, J=8.4 Hz, 1H), 0.42 (d, J=6.8 Hz, 1H), 7.27 (d, J=8.0 Hz, 1H), 6.81 (d, J=9.2 Hz, 1H), 5.12-5.10 (m, 1H), 4.76 (d, J=14.0 Hz, 1H), 4.56 (d, J=14.0 Hz, 1H), 4.37 (m, 2H), 3.51 (m, 4H), 3.34 (s, 3H), 2.78 (m, 2H), 2.63 (m, 4H), 2.22-2.18 (m, 2H), 2.18 (s, 3H), 2.09-2.08 (m, 2H), 1.27 (m, 2H).

LRMS (ESI) m/z: 932 [M+H]⁺.

### Example 24: Synthesis of(2S,4R)-1-((S)-3,3-dimethyl-245-(4-(5-((8-methyl-6-(2-methyl-5-(3-(trifluoromethyl)benzamido)phenyl)-7-oxo-5,6,7,8-tetrahydropyrimido[4,5-d]pyrimidin-2-yl)amino)pyridin-2-yl)piperazin-1-yl)pentanamido)butanoyl)-4-hydroxy-N—((S)-1-(4-(4-methylthiazol-5-v1)phenyl)ethyl)pyrrolidine-2-carboxamide (DB1113, 13) and (2S,4R)-1-((S)-3,3-dimethyl-2-(6-(4-(5-((8-methyl-6-(2-methyl-5-(3-(trifluoromethyl)benzamido)phenyl)-7-oxo-5,6,7,8-tetrahydropyrimido[4,5-d]pyrimidin-2-yl)amino)pyridin-2-yl)piperazin-1-yl)hexanamido)butanoyl)-4-hydroxy-N—((S)-1-(4-(4-methylthiazol-5-yl)phenyl)ethyl)pyrrolidine-2-carboxamide (DB1114, 14)

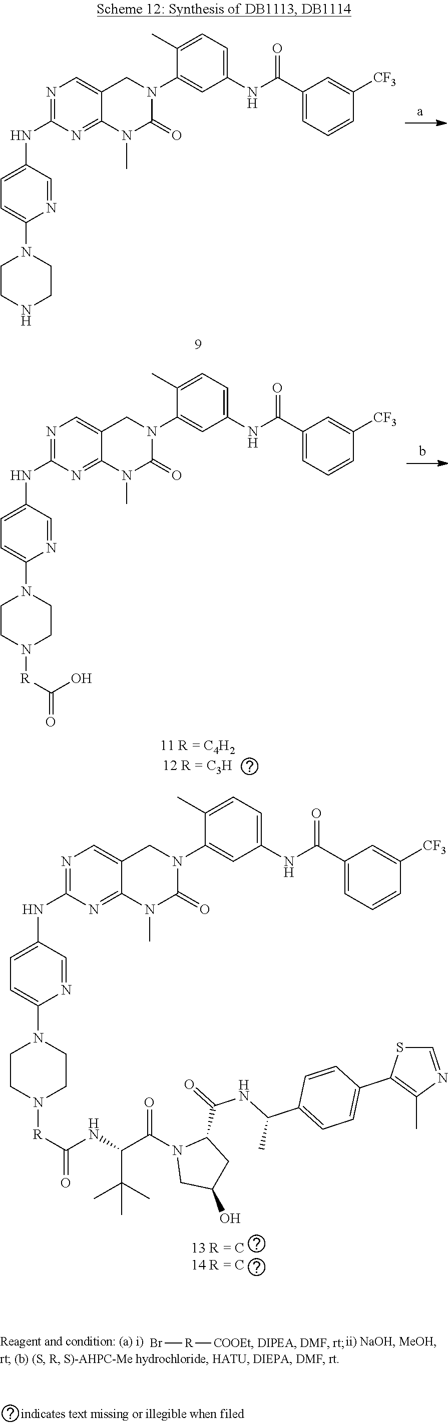

### General Procedure B for the Synthesis of Compound (11/12)

To a solution of compound 9 (1.0 eq) in DMF (5.0 mL) was added corresponding chain bearing bromo compound (3.0 eq), DIPEA (3.0 eq) at RT. The reaction mixture was then stirred for 30 minutes at 60° C., quenched with water, and diluted with EtOAc. The organic layer was washed with brine, dried over MgSO₄, filtered, and concentrated under reduced pressure. To a solution of crude intermediate ester in MeOH (10.0 mL) was added aqueous NaOH (5.0 eq) at 0° C. The reaction mixture was then stirred for 1 hour at RT, diluted with EtOAc, and neutralized with aqueous citric acid. The organic layer was washed with brine, dried over MgSO₄, filtered, and concentrated under reduced pressure. The residue was solidified by swirling in CH₂Cl₂/Et₂O and concentrated to give compound 11/12, which was used in the next reaction without any further purification.

### 5-(4-(5-((8-Methyl-6-(2-methyl-5-(3-(trifluoromethyl)benzamido)phenyl)-7-oxo-5,6,7,8-tetrahydropyrimido[4,5-d]pyrimidin-2-yl)amino)pyridin-2-yl)piperazin-1-yl)pentanoic acid (11)

Compound 9 (350 mg, 0.57 mmol) was converted to the target compound using general procedure B.

LRMS (ESI) m/z: 718 [M+H]⁺.

### 6-(4-(5-((8-Methyl-6-(2-methyl-5-(3-(trifluoromethyl)benzamido)phenyl)-7-oxo-5,6,7,8-tetrahydropyrimido[4,5-d]pyrimidin-2-yl)amino)pyridin-2-yl)piperazin-1-yl)hexanoic acid (12)

Compound 9 (394 mg, 0.64 mmol) was converted to the target compound using general procedure B.

LRMS (ESI) m/z: 732 [M+H]⁺.

### General Procedure C for the Synthesis of Compound (13/14)

To a solution of compound 11 or 12 (1.0 eq) in DMF (10 mL) was added (S,R,S)-AHPC-Me hydrochloride (1.2 eq), HATU (3.0 eq), and DIPEA (5.0 eq) at 0° C. The reaction mixture was then stirred for 30 minutes at 0° C., quenched with water, and diluted with EtOAc. The organic layer was washed with brine, dried over MgSO₄, filtered and concentrated under reduced pressure. The residue was purified by flash column chromatography (silica gel, 20% EtOAc/hexane) to give compound 13/14 (yield 49-52%).

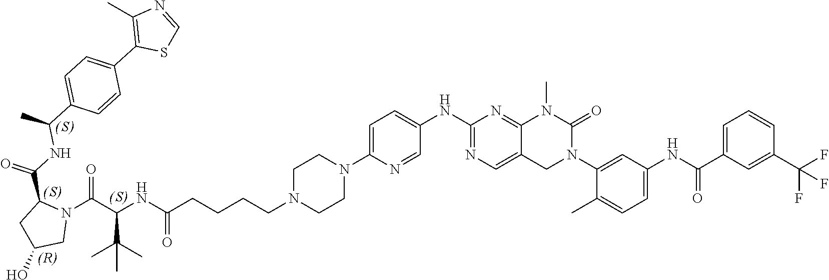

### DB1113

Compound 11 (121 mg, 0.17 mmol) was converted to the target compound using general procedure C. The residue was purified by flash column chromatography (silica gel, 20% EtOAc/hexane) to give compound DB1113 (13) (95 mg, yield 49%).

¹H NMR (400 MHz, Acetone-d6) δ 9.86 (s, 1H), 8.83 (s, 1H), 8.52 (s, 1H), 8.46 (d, J=10.9 Hz, 1H), 8.29 (d, J=9.1 Hz, 2H), 8.06 (s, 1H), 8.01 (d, J=9.0 Hz, 1H), 7.91 (d, J=7.5 Hz, 1H), 7.86 (s, 1H), 7.81-7.61 (m, 3H), 7.49-7.37 (m, 4H), 7.26 (d, J=8.1 Hz, 1H), 7.19-7.05 (m, 1H), 6.78 (d, J=9.2 Hz, 1H), 5.12-4.98 (m, 1H), 4.79-4.40 (m, 5H), 3.87 (d, J=10.6 Hz, 1H), 3.70 (d, J=7.0 Hz, 1H), 3.52-3.38 (m, 4H), 3.33 (s, 3H), 2.54-2.45 (m, 4H), 2.45 (s, 3H), 2.39-2.21 (m, 4H), 2.16 (s, 3H), 2.13-2.06 (m, 3H), 1.70-1.45 (m, 4H), 1.44 (d, J=6.9 Hz, 3H), 1.02 (s, 9H).

LRMS (ESI) m/z: 1145 [M+H]⁺.

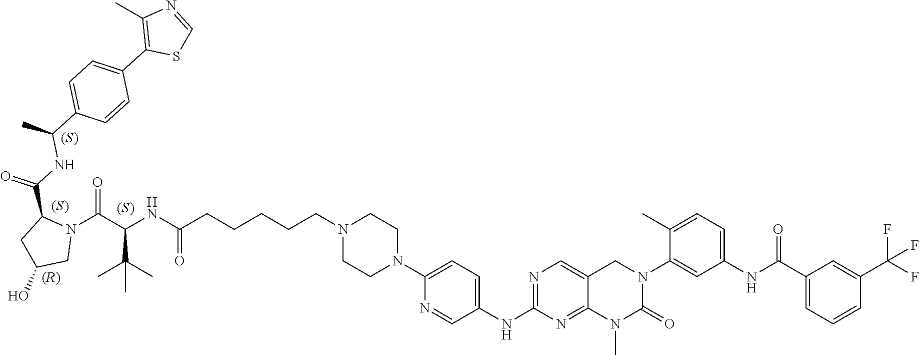

### DB1114

Compound 12 (118 mg, 0.16 mmol) was converted to the target compound using general procedure C. The residue was purified by flash column chromatography (silica gel, 20% EtOAc/hexane) to give compound DB1114 (14) (97 mg, yield 52%).

¹H NMR (400 MHz, Acetone-d₆) δ 9.86 (s, 1H), 8.83 (s, 1H), 8.52 (s, 1H), 8.46 (s, 1H), 8.33-8.24 (m, 2H), 8.06 (s, 1H), 8.02 (d, J=9.1 Hz, 1H), 7.91 (d, J=7.5 Hz, 1H), 7.86 (s, 1H), 7.81-7.70 (m, 2H), 7.66 (d, J=8.3 Hz, 1H), 7.50-7.37 (m, 4H), 7.27 (d, J=8.5 Hz, 1H), 7.14 (d, J=9.1 Hz, 1H), 6.80 (d, J=9.2 Hz, 1H), 5.10-4.97 (m, 1H), 4.76 (d, J=13.9 Hz, 1H), 4.69-4.51 (m, 3H), 4.46 (s, i H), 3.87 (d, J=11.0 Hz, 1H), 3.70 (dd, J=10.8, 3.8 Hz, 1H), 3.56-3.42 (m, 4H), 3.34 (s, 3H), 2.60-2.51 (m, 4H), 2.46 (s, 3H), 2.42-2.34 (m, 2H), 2.33-2.30 (m, 2H), 2.17 (s, 3H), 2.12-2.04 (m, 3H), 1.68-1.50 (m, 4H), 1.44 (d, J=6.9 Hz, 3H), 1.39-1.27 (m, 2H), 1.02 (s, 9H).

LRMS (ESI) m/z: 1158 [M+H]⁺.

### Example 25: Synthesis of N-(3-(7-((6-(4-((1-(5-((2-(2,6-Dioxopiperidin-3-yl)-1,3-dioxoisoindolin-4-yl)oxy)pentyl)-1H-1,2,3-triazol-4-yl)methyl)piperazin-1-v)pyridin-3-yl)amino)-1-methyl-2-oxo-1,4-dihydropyrimido[4,5-d]pyrimidin-3(2H)-yl)-4-methylphenyl)-3-(trifluoromethyl)benzamide (DB0646, 16)

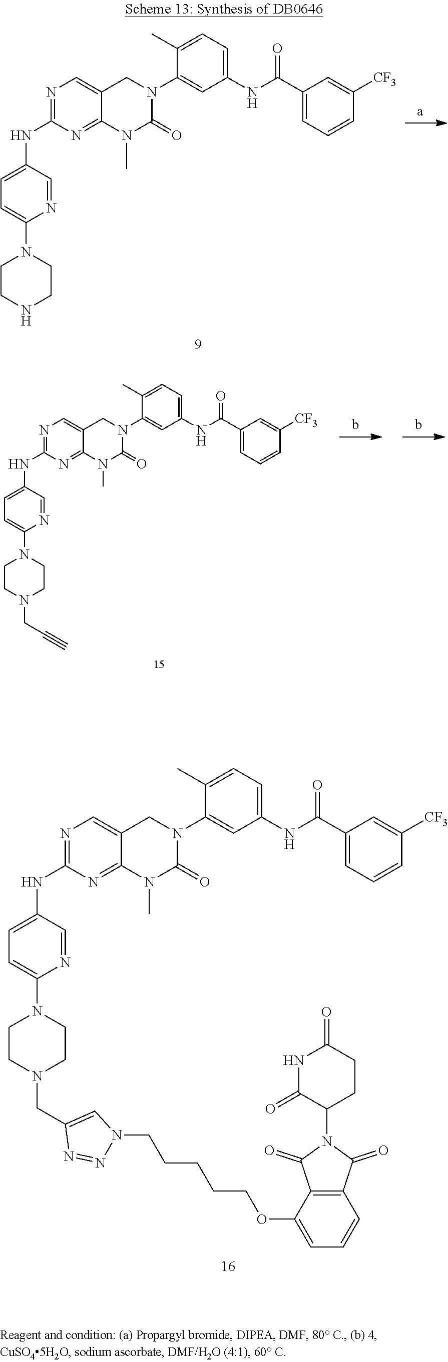 [SMILES: Cc1ccc(NC(=O)c2cccc(C(F)(F)F)c2)cc1N1Cc2cnc(Nc3ccc(N4CCNCC4)nc3)nc2N(C)C1=O ; C#CCN1CCN(c2ccc(Nc3ncc4c(n3)N(C)C(=O)N(c3cc(NC(=O)c5cccc(C(F)(F)F)c5)ccc3C)C4)cn2)CC1 ; Cc1ccc(NC(=O)c2cccc(C(F)(F)F)c2)cc1N1Cc2cnc(Nc3ccc(N4CCN(Cc5cn(CCCCCOc6cccc7c6C(=O)N(C6CCC(=O)NC6=O)C7=O)nn5)CC4)nc3)nc2N(C)C1=O]

### N-(4-Methyl-3-(1-methyl-2-oxo-7-((6-(4-(prop-2-yn-1-yl)piperazin-1-yl)pyridin-3-yl)amino)-1,4-dihydropyrimido[4,5-d]pyrimidin-3(2H1)-yl)phenyl)-3(trifluoromethyl)benzamide (15)

To a solution of compound 9 (200 mg, 0.32 mmol) in DMF (2.0 mL) was added propargyl bromide (38.5 mg, 0.32 mmol) and DIPEA (0.56 mL, 3.24 mmol) at RT. The reaction mixture was then stirred for 1 hour at 80° C., quenched with water, and diluted with EtOAc. The organic layer was washed with brine, dried over MgSO₄, filtered, and concentrated under reduced pressure. The resulting residue was purified by flash column chromatography (silica gel, 4-7% MeOH/CH₂Cl₂) to give compound 15 (168 mg, yield 79%).

¹H NMR (400 MHz, Acetone-d₆) δ 9.78 (s, 1H), 8.48 (d, J=2.4 Hz, 1H), 8.40 (s, 1H), 8.26 (s, 1H), 8.25 (d, J=7.2 Hz, 1H), 8.02 (s, 1H), 7.98 (dd, J=7.2, 2.4 Hz, 1H), 7.87 (d, J=6.0 Hz, 1H), 7.82 (d, J=1.6 Hz, 1H), 7.72 (t, J=6.4 Hz, 1H), 7.63 (dd, J=6.4, 1.6 Hz, 1H), 7.21 (d, J=6.4 Hz, 1H), 6.77 (d, J=7.6 Hz, 1H), 4.71 (d, J=10.0 Hz 1H), 4.51 (d, J=10.0 Hz, 1H), 3.47 (m, 4H), 3.31 (s, 3H), 3.30 (s, 2H), 2.67 (t, J=0.8 Hz, 1H), 2.58 (m, 4H), 2.10 (s, 3H).

LRMS (ESI) m/z: 656 [M+H]⁺.

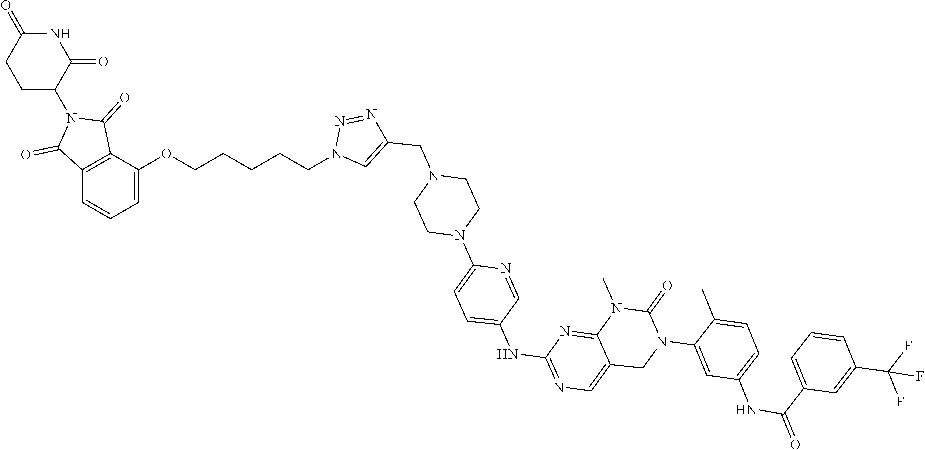 [SMILES: Cc1ccc(NC(=O)c2cccc(C(F)(F)F)c2)cc1N1Cc2cnc(Nc3ccc(N4CCN(Cc5cn(CCCCCOc6cccc7c6C(=O)N(C6CCC(=O)NC6=O)C7=O)nn5)CC4)nc3)nc2N(C)C1=O]

### DB0646

To a solution of compound 15 (30 mg, 0.05 mmol) in DMF/H₂O (4:1, 2.0 mL) was added compound 4 (1.2 eq), sodium ascorbate (1.5 eq), and CuSO₄·5H₂O (1.5 eq) at RT. The reaction mixture was then stirred for 3 hours at 60° C. quenched with water and diluted with EtOAc. The organic layer was washed with brine, dried over MgSO₄, filtered and concentrated under reduced pressure. The residue was purified by flash column chromatography on silica gel (4-7% MeOH/CH₂Cl₂) to give compound 16 (38 mg, yield 79%).

¹H NMR (400 MHz, Acetone-d₆) δ 10.39 (s, 1H), 9.83 (s, 1H), 8.50 (s, 1H), 8.42 (s, 1H), 8.31 (s, 1H), 8.28 (s, 1H), 8.07 (s, 1H), 8.01 (d, J=8.0 Hz, 1H), 7.92 (d, J=8.0 Hz, 1H), 7.88 (m, 1H), 7.78 (dd, J=16.0, 8.0 Hz, 2H), 7.66 (d, J=8.0 Hz, 11H), 7.44 (dd, J=16.0, 8.0 Hz, 2H), 7.28 (d, J=8.0 Hz, 1H), 6.78 (d, J=8.0 Hz, 1H), 5.10 (dd, J=16.0, 8.0 Hz, 2H), 4.77 (d, J=14.0 Hz, 1H), 4.56 (d, J=14.0 Hz, 1H), 4.47-4.44 (m, 2H), 4.26 (m, 2H), 3.67 (s, 2H), 3.48 (s, 3H), 3.34 (s, 3H), 2.59 (s, 3H), 2.19 (s, 3H), 1.92-1.89 (m, 2H), 1.62-1.59 (m, 2H).

LRMS (ESI) m/z: 1042 [M+H]⁺.

### Example 26: Synthesis of N-(3-(7-((6-(4-((1-(5-(3-(2-((S)-1-((S)-2-cyclohexyl-2-((S)-2-(methylamino)propanamido)acetyl)pyrrolidin-2-yl)thiazole-4-carbonyl)phenoxy)pentyl)-1H-1,2,3-triazol-4-yl)methyl)piperazin-1-yl)pyridin-3-yl)amino)-1-methyl-2-oxo-1,4-dihydropyrimido[4,5-d]pyrimidin-3(2H)-yl)-4-methylphenyl)-3-(trifluoromethyl)benzamide (DB118450, 20)

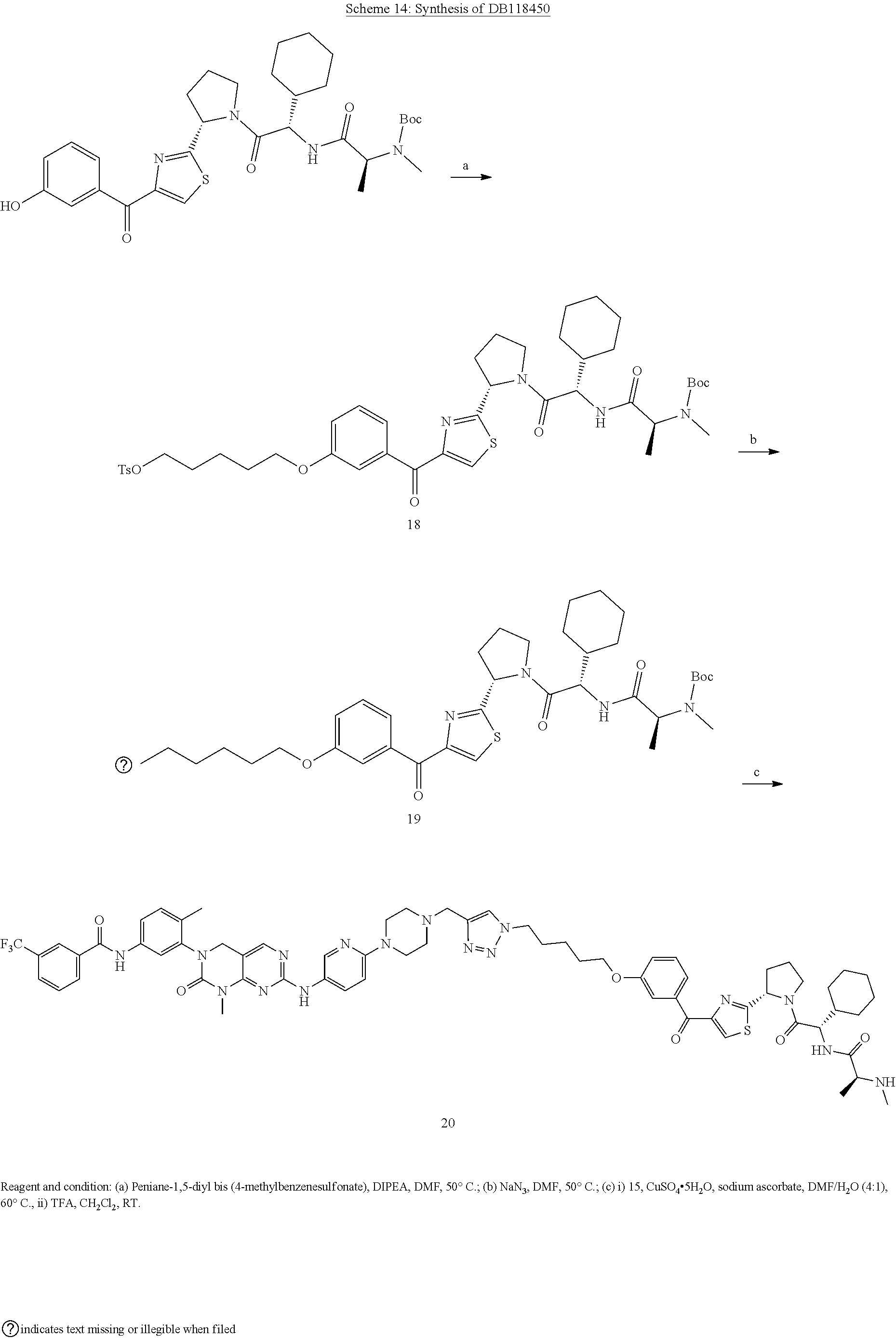

### 5-(3-(2-((S)-1-((S)-2-(2-((tert-Butoxycarbonyl)(methyl)amino)acetamido)-2-cyclohexylacetyl)pyrrolidin-2-yl)thiazole-4-carbonyl)phenoxy)pentyl 4-methylbenzenesulfonate (18)

To a solution of compound 17 (100 mg, 0.17 mmol) in DMF (10.0 mL) was added pentane-1,5-diyl bis(4-methylbenzenesulfonate) (206 mg, 0.50 mmol) and K₂CO₃ (46 mg, 0.33 mmol) at RT. The reaction mixture was then stirred for 3 hours at 50° C., quenched with water, and diluted with EtOAc. The organic layer was washed with brine, dried over MgSO₄, filtered, and concentrated under reduced pressure. The residue was purified by flash column chromatography (silica gel, 20-30% THF/hexane) to give compound 18 (131 mg, yield 93%).

LRMS (ESI) m/z: 826 [M+H]⁺.

### tert-Butyl (2-(((S)-2-((S)-2-(4-(3-((5-azidopentyl)oxy)benzoyl)thiazol-2-yl)pyrrolidin-1-yl)-1-cyclohexyl-2-oxoethyl)amino)-2-oxoethyl)(methyl)carbamate (19)

To a solution of compound 18 (100 mg, 0.12 mmol) in DMF (10.0 mL) was added NaN₃ (46 mg, 0.72 mmol) at RT. The reaction mixture was then stirred for 2 hours at 50° C., quenched with water, and diluted with EtOAc. The organic layer was washed with brine, dried over MgSO₄, filtered, and concentrated under reduced pressure. The residue was purified by flash column chromatography (silica gel, 15-20% THF/hexane) to give compound 19 (65 mg, yield 80%).

LRMS (ESI) m/z: 697 [M+H]⁺.

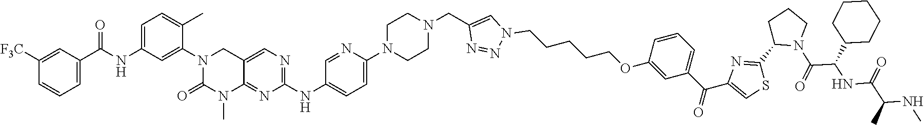 [SMILES: CN[C@@H](C)C(=O)N[C@H](C(=O)N1CCC[C@H]1c1nc(C(=O)c2cccc(OCCCCCn3cc(CN4CCN(c5ccc(Nc6ncc7c(n6)N(C)C(=O)N(c6cc(NC(=O)c8cccc(C(F)(F)F)c8)ccc6C)C7)cn5)CC4)nn3)c2)cs1)C1CCCCC1]

### DB118450

To a solution of compound 15 (61 mg, 0.09 mmol) in DMF/H₂O (4:1, 5.0 mL) was added compound 19 (99 mg, 0.14 mmol), sodium ascorbate (28.0 mg, 0.14 mmol) and CuSO₄·5H₂O (35 mg, 0.14 mmol) at RT. The reaction mixture was then stirred for 30 minutes at 60° C., quenched with water and diluted with EtOAc. The organic layer was washed with brine, dried over MgSO₄, filtered, and concentrated under reduced pressure. The residue was solidified by swirling in CH₂Cl₂/Et₂O, and concentrated under reduced pressure to give a white solid. To a solution of the resulting residue in CH₂Cl₂(10.0 mL) was added TFA (10.0 equiv) at 0° C. The reaction mixture was then stirred for 36 hours at RT, diluted with EtOAc, and neutralized with aqueous NaHCO₃. The organic layer was washed with brine, dried over MgSO₄, filtered, and concentrated under reduced pressure. The residue was purified by flash column chromatography (silica gel, 4-7% MeOH/CH₂Cl₂) to give compound DB118450 (20) (90 mg, 50% overall yield for 2 steps).

¹H NMR (400 MHz, Methanol-d₄) δ 8.48 (s, 1H), 8.32 (d, J=6.8 Hz, 2H), 8.27 (d, J=7.8 Hz, 1H), 8.08 (s, 1H), 8.03 (s, 1H), 8.00-7.87 (m, 2H), 7.87-7.71 (m, 3H), 7.72-7.60 (m, 2H), 7.47 (t, J=8.0 Hz, 1H), 7.40 (d, J=8.5 Hz, 1H), 7.23 (d, J=5.6 Hz, 1H), 6.84 (d, J=9.5 Hz, 1H), 5.51 (d, J=7.0 Hz, 1H), 4.82 (d, J=14.1 Hz, 1H), 4.71-4.46 (m, 4H), 2H), 3.89-3.75 (m, 2H), 3.47-3.48 (m, 4H), 3.46 (s, 3H), 3.41-3.39 (m, 4H), 3.30-3.19 (m, 1H), 2.74-2.62 (m, 4H), 2.48-2.36 (m, 2H), 2.33-2.15 (m, 7H), 2.14-2.04 (m, 2H), 1.98-1.69 (m, 7H), 1.65-1.44 (m, 3H), 1.42-1.21 (m, 6H).

LRMS (ESI) m/z: 633 [M/2+H]⁺.

### Example 27: Synthesis of-((9-chloro-7-(2-fluoro-6-methoxyphenyl)-5H-benzo[c]pyrimido[4,5-e]azepin-2-yl)amino)-N-(1-((2-(2,6-dioxopiperidin-3-yl)-1,3-dioxoisoindolin-4-yl)oxy)-2-oxo-7,10,13-trioxa-3-2174 azahexadecan-16-yl)-2-methoxybenzamide (dAURK-4)

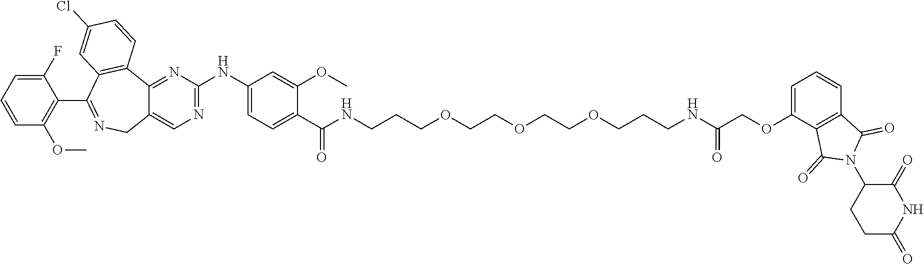 [SMILES: COc1cc(Nc2ncc3c(n2)-c2ccc(Cl)cc2C(c2c(F)cccc2OC)=NC3)ccc1C(=O)NCCCOCCOCCOCCCNC(=O)COc1cccc2c1C(=O)N(C1CCC(=O)NC1=O)C2=O]

dAURK-4

N-(3-(2-(2-(3-aminopropoxy)ethoxy)ethoxy)propyl)-2-((2-(2,6-dioxopiperidin-3-yl)-1,3-dioxoisoindolin-4-yl)oxy)acetamide trifluoroacetate salt (12.4 mg, 0.0191 mmol, 1 eq) was added to 4-((9-chloro-7-(2-fluoro-6-methoxyphenyl)-5H-benzo[c]pyrimido[4,5-e]azepin-2-yl)amino)-2-methoxybenzoic acid (MLN8237) (9.9 mg, 0.0191 mmol, 1 eq) as a solution in DMF (0.191 mL). DIPEA (0.010 mL, 0.0572 mmol, 3 eq) was added, followed by HATU (7.3 mg, 0.191 mmol, 1 eq). After 24 hours, the mixture was diluted with EtOAc and washed sequentially with saturated sodium bicarbonate, water, and brine. The organic layer was dried over sodium sulfate, filtered and concentrated under reduced pressure. Purification by column chromatography (0-10% MeOH/DCM) gave the desired product dAURK-4 as a yellow solid (11.03 mg, 0.0107 mmol, 56%).

¹H NMR (400 MHz, 1:1 MeOD:CDCl₃) δ 8.52 (s, 1H), 8.27 (d, J=8.5 Hz, 1H), 7.96 (d, J=8.7 Hz, 1H), 7.93 (d, J=1.9 Hz, 1H), 7.74-7.68 (m, 1H), 7.61 (dd, J=8.5, 2.1 Hz, 1H), 7.56 (s, 2H), 7.48 (d, J=7.4 Hz, 1H), 7.38-7.21 (m, 4H), 6.80 (broad s, 2H), 5.01 (dd, J=11.9, 6.1 Hz, 1H), 4.63 (s, 2H), 3.98 (s, 6H), 3.57 (dddd, J=27.7, 15.3, 10.9, 7.0 Hz, 13H), 3.41 (t, J=6.8 Hz, 2H), 2.85-2.68 (m, 3H), 2.12 (d, J=8.3 Hz, 1H), 1.85 (dp, J=19.9, 7.1 Hz, 4H).

LC/MS (ESI) m/z: 1035.6 [M+H]⁺.

### Example 28: Synthesis of N-(2-chloro-6-methylphenyl)-2-((6-(4-(2-((4-(2-((2-(2,6-dioxopiperidin-3-yl)-1,3-dioxoisoindolin-4-yl)oxy)acetamido)butyl)amino)-2-oxoethyl)piperazin-1-yl)-2-methylpyrimidin-4-yl)amino)thiazole-5-carboxamide (DB-3-291)

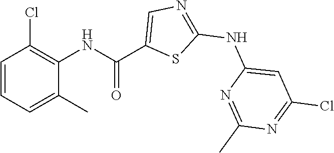 [SMILES: Cc1nc(Cl)cc(Nc2ncc(C(=O)Nc3c(C)cccc3Cl)s2)n1]

### 2-((6-chloro-2-methylpyrimidin-4-yl)amino)-N-(2-chloro-6-methylphenyl)thiazole-5-carboxamide

2-amino-N-(2-chloro-6-methylphenyl)thiazole-5-carboxamide (1.34 g, 5 mmol, 1 eq) and 4,6-dichloro-2-methylpyrimidine (0.978 g, 6 mmol, 1.2 eq) were dissolved in THF (17 mL), and the mixture was cooled to 0° C. After 0.5 hours, sodium tert-butoxide (1.68 g, 17.5 mmol, 3.5 eq) was added. After 2 hours, 1M HCl was added to adjust pH to −5-6. The mixture was stirred for 15 minutes, and then filtered. There resulting solid was washed twice with methanol and once with water, and then air dried to give the desired product (1.26 g, 3.20 mmol, 64%) as a white solid, which was used in the next step without further purification.

¹H NMR (500 MHz, DMSO-d₆) δ 12.24 (s, 1H), 10.00 (s, 1H), 8.31 (s, 1H), 7.44-7.39 (m, 1H), 7.27 (ddd, J=19.8, 12.4, 7.0 Hz, 2H), 6.93 (s, 1H), 2.59 (s, 3H), 2.24 (s, 3H).

LC/MS (ESI) m/z: 394.25 [M+H]⁺

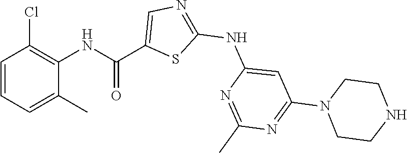 [SMILES: Cc1nc(Nc2ncc(C(=O)Nc3c(C)cccc3Cl)s2)cc(N2CCNCC2)n1]

### N-(2-chloro-6-methylphenyl)-2-((2-methyl-6-(piperazin-1-yl)pyrimidin-4-yl)amino)thiazole-5-carboxamide

To a solution of 2-((6-chloro-2-methylpyrimidin-4-yl)amino)-N-(2-chloro-6-methylphenyl)thiazole-5-carboxamide (0.56 g, 1.42 mmol, 1 eq) and piperazine (1.22 g, 14.2 mmol, 10 eq) in dioxane (18 mL, 0.08 M) was added DIPEA (0.49 mL, 2.84 mmol, 2 eq). The mixture was heated to 100° C. for 20 hours. The mixture was cooled to RT and concentrated under reduced pressure. The crude product was triturated twice with 1:1 MeOH:water (25 ml), once with 1:1 MeOH:Et₂O (25 mL), and with Et₂O (25 mL). The washes were then concentrated, and triturated three times with 20 mL of 1:4 MeOH:water to isolate additional material, which was combined with the previously isolated material. The desired product was isolated as a white solid (533.9 mg, 1.20 mmol, 85%) and used without further purification.

¹H NMR (500 MHz, DMSO-d6) δ 9.86 (s, 1H), 8.21 (s, 1H), 7.46-7.34 (m, 1H), 7.27 (dt, J=15.3, 7.1 Hz, 2H), 6.02 (s, 1H), 3.44 (d, J=4.6 Hz, 4H), 2.79-2.70 (m, 4H), 2.40 (s, 3H), 2.24 (s, 3H).

LC/MS (ESI) m/z: 444.34 [M+H]⁺.

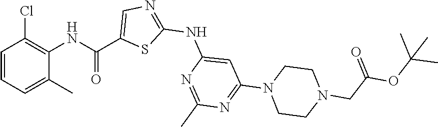 [SMILES: Cc1nc(Nc2ncc(C(=O)Nc3c(C)cccc3Cl)s2)cc(N2CCN(CC(=O)OC(C)(C)C)CC2)n1]

### tert-Butyl 2-(4-(6-((5-((2-chloro-6-methylphenyl)carbamoyl)thiazol-2-yl)amino)-2-methylpyrimidin-4-yl)piperazin-1-yl)acetate

To a solution of N-(2-chloro-6-methylphenyl)-2-((2-methyl-6-(piperazin-1-yl)pyrimidin-4-yl)amino)thiazole-5-carboxamide (236 mg, 0.532 mmol, 1 eq) in DMF (5.3 mL, 0.1 M) was added triethylamine (0.222 mL, 1.59 mmol, 3 eq), followed by tert-butyl bromoacetate (0.118 mL, 0.797 mmol, 1.5 eq). The reaction was stirred at RT for 15 hours. The mixture was diluted with saturated aqueous sodium bicarbonate (25 mL) and then extracted three times with EtOAc. The combined organic layers were washed three times with brine, then dried with sodium sulfate, filtered, and concentrated under reduced pressure to obtain desired product, which was used in the next step without further purification.

¹H NMR (500 MHz, DMSO-d₆) δ 11.44 (s, 1H), 9.86 (s, 1H), 8.21 (s, 1H), 7.40 (d, J=6.4 Hz, 1H), 7.27 (dt, J=15.3, 7.1 Hz, 2H), 6.05 (s, 1H), 3.52 (s, 4H), 3.17 (s, 2H), 2.61-2.54 (m, 4H), 2.40 (s, 3H), 2.24 (s, 3H), 1.42 (s, 9H).

LC/MS (ESI) m/z: 558.46 [M+H]⁺.

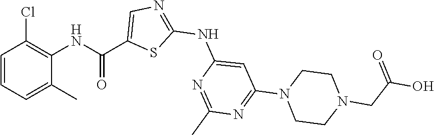 [SMILES: Cc1nc(Nc2ncc(C(=O)Nc3c(C)cccc3Cl)s2)cc(N2CCN(CC(=O)O)CC2)n1]

### 2-(4-(6-((5-((2-chloro-6-methylphenyl)carbamoyl)thiazol-2-yl)amino)-2-methylpyrimidin-4-yl)piperazin-1-yl)acetic acid

A solution of tert-Butyl 2-(4-(6-((5-((2-chloro-6-2237 methylphenyl)carbamoyl)thiazol-2-yl)amino)-2-methylpyrimidin-4-yl)piperazin-1-yl)acetate in DCM (50 mL) and TFA (10 mL) was stirred at room temperature for 18 hours. The mixture was concentrated under reduced pressure and precipitated with Et20. The desired product was dried under reduced pressure and isolated as a white solid (257 mg, 0.512 mmol, 98% yield), which was used in the next step without further purification.

LC/MS (ESI) m/z: 502.37 ([M+H]⁺.

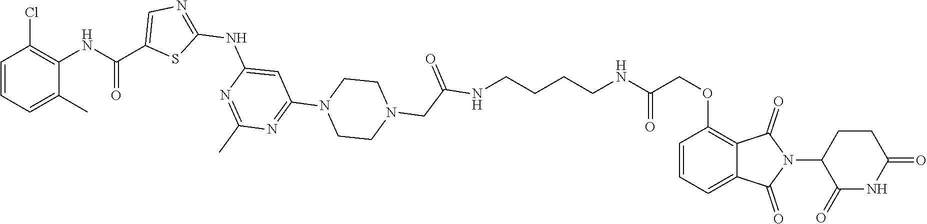 [SMILES: Cc1nc(Nc2ncc(C(=O)Nc3c(C)cccc3Cl)s2)cc(N2CCN(CC(=O)NCCCCNC(=O)COc3cccc4c3C(=O)N(C3CCC(=O)NC3=O)C4=O)CC2)n1]

### DB-3-291

N-(4-aminobutyl)-2-((2-(2,6-dioxopiperidin-3-yl)-1,3-dioxoisoindolin-4-yl)oxy)acetamide trifluoroacetate salt (12.9 mg, 0.025 mmol, 1 eq) was added to 2-(4-(6-((5-((2-chloro-6-2249 methylphenyl)carbamoyl)thiazol-2-yl)amino)-2-methylpyrimidin-4-yl)piperazin-1-yl)acetic acid (15.4 mg, 0.025 mmol, 1 eq) as a 0.1M solution in DMF (0.25 mL) at room temperature. DIPEA (0.131 mL, 0.075 mmol, 3 eq) was added, followed by HATU (9.5 mg, 0.025 mmol, 1 eq), and the mixture was stirred for 13 hours. The crude mixture was diluted with MeOH and purified by preparative HPLC. The desired product was isolated as a yellow solid (11.16 mg, 0.0112 mmol, 45%).

¹H NMR (500 MHz, Methanol-d₄) δ 8.17 (s, 1H), 7.82 (dd, J=8.4, 7.4 Hz, 1H), 7.55 (d, J=7.2 Hz, 1H), 7.45 (d, J=8.4 Hz, 1H), 7.38-7.34 (m, 1H), 7.28-7.22 ((m, 2H), 6.16 (s, 1H), 5.14 (dd, J=12.7, 5.5 Hz, 1H), 4.78 (s, 2H), 3.95 (s, 6H), 3.52-3.39 (m, 4H), 3.38-3.32 (m, 4H), 2.88 (ddd, J=17.5, 13.9, 5.2 Hz, 1H), 2.80-2.68 (M, 2H), 2.52 (s, 3H), 2.32 (s, 3H), 2.16 (dtd, J=13.0, 5.7, 2.7 Hz, 1H), 1.67-1.54 (m, 4H).

LC/MS (ESI) m/z: 886.61 [M+H]⁺.

### Example 29: Synthesis of N-(2-(2-((4-(2-(tert-butyl)-4-(3-((2,6-difluorophenyl)sulfonamido)-2-fluorophenyl)thiazol-5-yl)pyrimidin-2-yl)amino)ethoxy)ethyl)-3-(2-(2-((2-(2,6-dioxopiperidin-3-yl)-1,3-dioxoisoindolin-4-yl)amino)ethoxy)ethoxy)propenamide (DD-03-106-1)

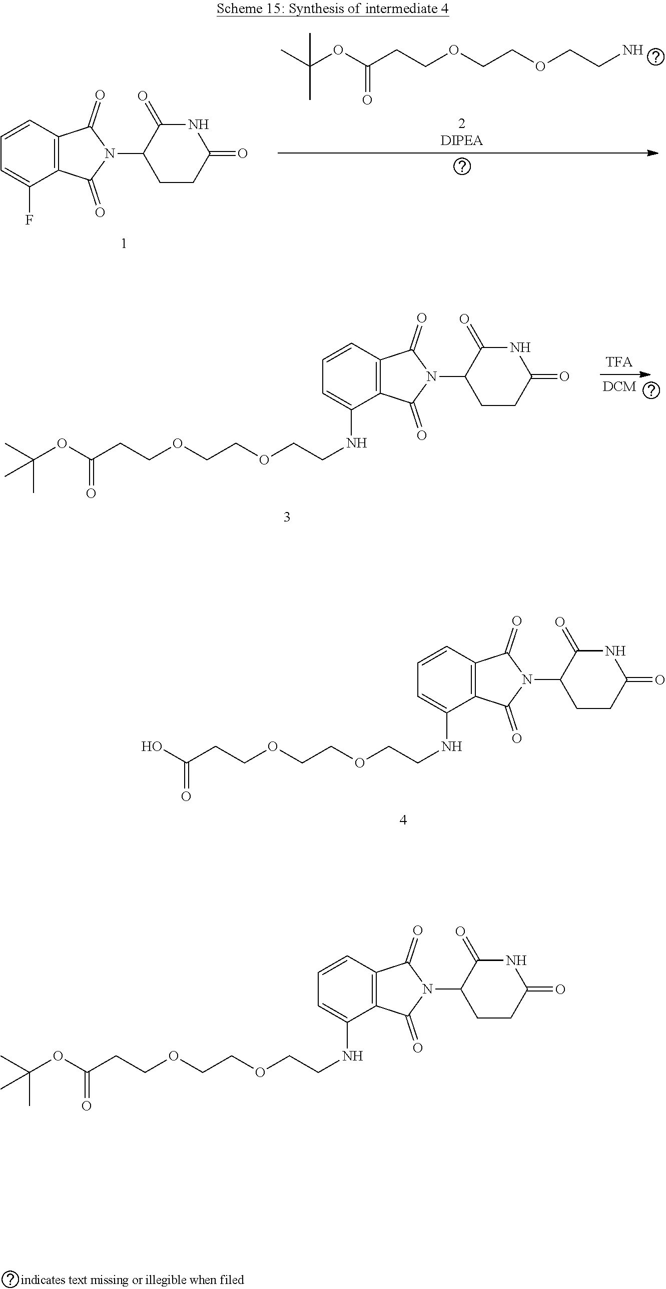 [SMILES: O=C1CCC(N2C(=O)c3cccc(F)c3C2=O)C(=O)N1 ; CC(C)(C)OC(=O)CCOCCOCCN ; CC(C)(C)OC(=O)CCOCCOCCNc1cccc2c1C(=O)N(C1CCC(=O)NC1=O)C2=O ; O=C(O)CCOCCOCCNc1cccc2c1C(=O)N(C1CCC(=O)NC1=O)C2=O ; CC(C)(C)OC(=O)CCOCCOCCNc1cccc2c1C(=O)N(C1CCC(=O)NC1=O)C2=O]

### tert-butyl 3-(2-(2-((2-(2,6-dioxopiperidin-3-yl)-1,3-dioxoisoindolin-4-yl)amino)ethoxy)ethoxy)propanoate (3)

A solution of compounds 1 (455 mg, 1.74 mmol) and 2 (405 mg, 1.74 mmol) and DIPEA (1.21 mL, 6.94 mmol) in dimethylacetamide (DMA) (2 mL) was stirred at 90° C. for 20 hours. The reaction was diluted with H₂O (10 mL) and extracted with ethyl acetate (4×20 mL). The combined organic layers were washed with H₂O (10 mL) and brine (10 mL). The organic layer was dried over sodium sulfate, filtered, and concentrated under reduced pressure. The crude residue was purified by column chromatography (0-70% ethyl acetate/hexanes) to give the desired product as a yellow solid (250 mg, 0.51 mmol, 29%).

LC/MS (ESI) m/z: 490.5[M+H]⁺.

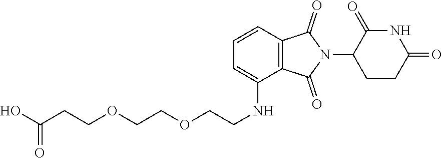 [SMILES: O=C(O)CCOCCOCCNc1cccc2c1C(=O)N(C1CCC(=O)NC1=O)C2=O]

### 3-(2-(2-((2-(2,6-dioxopiperidin-3-yl)-1,3-dioxoisoindolin-4-yl)amino)ethoxy)ethoxy) propanoic acid (4)

A solution of compound 3 (250 mg, 0.51 mmol) in TFA (3 mL) and DCM (3 mL) was stirred at RT for 1 hour. The reaction mixture was concentrated under reduced pressure to obtain the product (4), which was used in the next step without further purification.

LC/MS (ESI) m/z: 434.4 [M+H]⁺

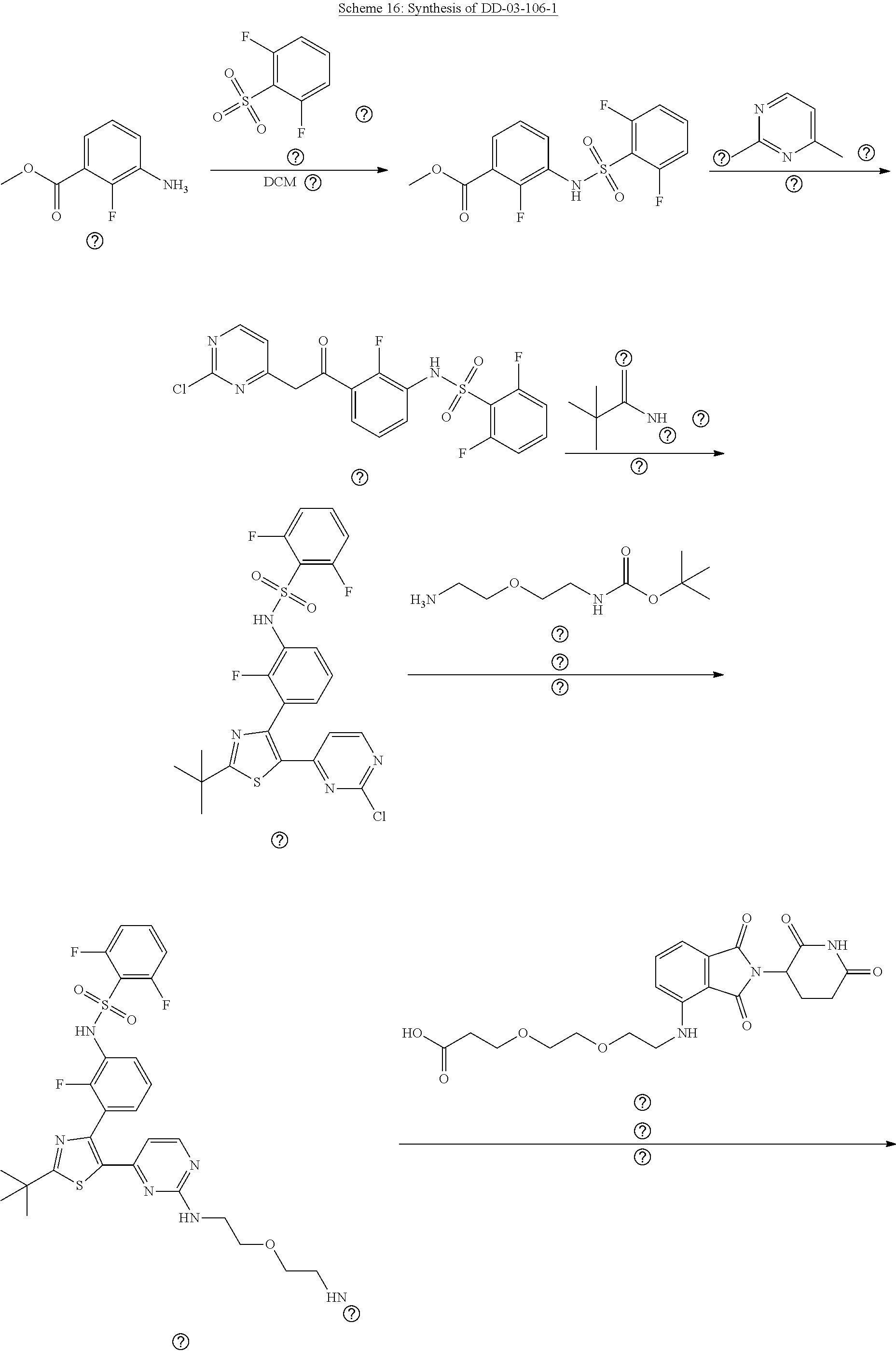

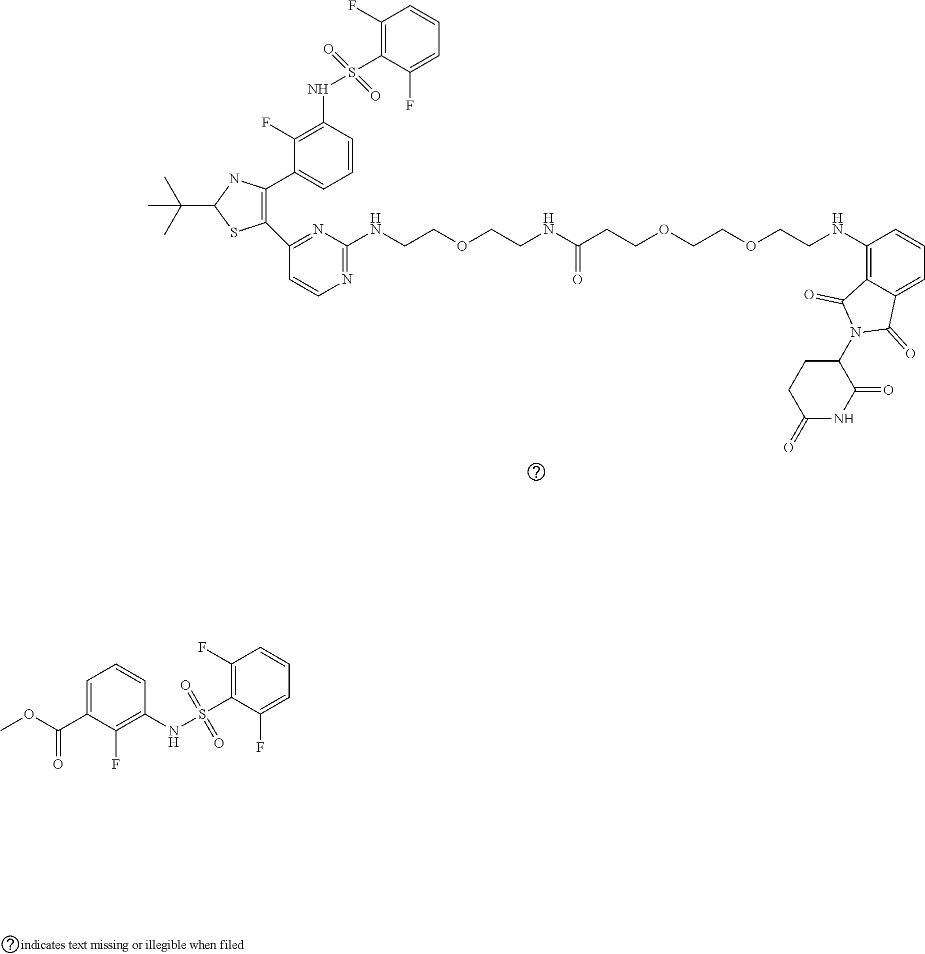 [SMILES: CC(C)(C)C1NC(c2cccc(NS(=O)(=O)c3c(F)cccc3F)c2F)=C(c2ccnc(NCCOCCNC(=O)CCOCCOCCNc3cccc4c3C(=O)N(C3CCC(=O)NC3=O)C4=O)n2)S1 ; COC(=O)c1cccc(NS(=O)(=O)c2c(F)cccc2F)c1F]

### Methyl 3-((2,6-difluorophenyl)sulfonamido)-2-fluorobenzoate (7)

Compound 6 (2.4 mL, 17.7 mmol) was added dropwise to a solution of compound 5 (3.0 g, 17, mmol) and pyridine (1.6 mL, 19.5 mmol) in DCM (15 mL). The reaction mixture was stirred at RT for 16 hours. The reaction mixture was concentrated under reduced pressure and purified by column chromatography (30-100% hexanes/EtOAc) to afford the desired product (7) (5.79 g, 16.7 mmol, 94%).

LC/MS (ESI) m/z: 346.3 [M+H]⁺.

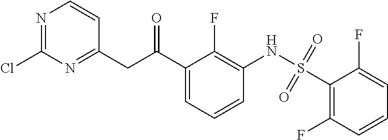 [SMILES: O=C(Cc1ccnc(Cl)n1)c1cccc(NS(=O)(=O)c2c(F)cccc2F)c1F]

### N-(3-(2-(2-chloropyrimidin-4-yl)acetyl)-2-fluorophenyl)-2,6-difluorobenzenesulfonamide (8)

To a solution of compound 7 (450 mg, 1.30 mmol) in anhydrous THF was added sodium bis(trimethylsilyl)amide (NaHDMS) in THF (4.17 mL, 4.17 mmol) at 0° C. under N₂. A solution of 2-Chloro-4-methylpyrimidine (217 mg, 1.69 mmol) in THF (3 mL) was added to the mixture, and the reaction was stirred at 0° C.-RT for 1 hour. The reaction mixture was cooled to 0° C. before dropwise addition of 6 M HCl (10 mL). The mixture was extracted with ethyl acetate (3×50 mL). The combined organic layers were washed with brine (10 mL), dried over sodium sulfate, filtered, and concentrated under reduced pressure. The crude residue was purified by column chromatography (0-70% ethyl acetate/hexanes) to give the desired product (8) as a yellow solid (140 mg, 0.32 mmol, 24%).

LC/MS (ESI) m/z: 442.9 [M+H]⁺.

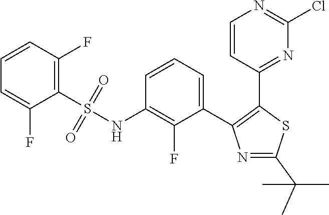 [SMILES: CC(C)(C)c1nc(-c2cccc(NS(=O)(=O)c3c(F)cccc3F)c2F)c(-c2ccnc(Cl)n2)s1]

### N-(3-(2-(tert-butyl)5-(2-chloropyrimidin-4-yl)thiazol-4-yl)-2-fluorophenyl)-2,6-difluorobenzenesulfonamide (9)

To a stirred solution of compound 8 (1.51 g, 3.42 mmol) in anhydrous DMA under N2 was added NBS (635 mg, 3.57 mmol). The reaction mixture was stirred at RT for 15 minutes, followed by the addition of 2,2-dimethylpropanethioamide (401 mg, 3.42 mmol). The reaction was heated at 80° C. for 2 hours, allowed to cool to RT, diluted with H₂O (60 mL), and extracted with EtOAc (4×120 mL). The combined organic layers were washed with H₂O (5×5 mL), dried over sodium sulfate, filtered, and concentrated under reduced pressure. The crude residue was purified by column chromatography (0-70% EtOAc/hexanes) gave the desired product (620 mg, 1.15 mmol, 34%).

LC/MS (ESI) m/z: 540.0 [M+H]⁺.

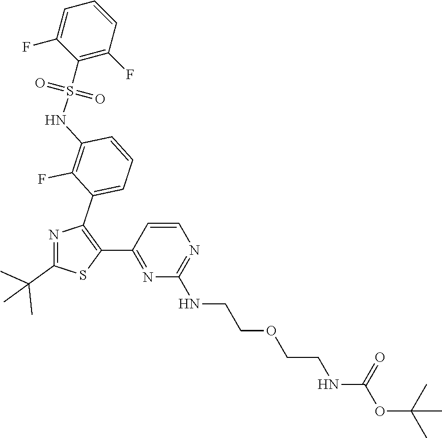 [SMILES: CC(C)(C)OC(=O)NCCOCCNc1nccc(-c2sc(C(C)(C)C)nc2-c2cccc(NS(=O)(=O)c3c(F)cccc3F)c2F)n1]

### tert-butyl (2-(2-((4-(2-(tert-butyl)-4-(3-((2,6-difluorophenyl)sulfonamido)-2-fluorophenyl)thiazol-5-yl)pyrimidin-2-yl)amino)ethoxy)ethyl)carbamate (11)

A mixture of compound 9 (86 mg, 0.16 mmol), compound 10 (63 mg, 0.32 mmol), sodium carbonate (34 mg, 0.32 mmol), and DIPEA (56 μL, 0.32 mmol) in N-methyl-2-pyrrolidone (NMP) (80 μL) stirred at 100° C. for 1 hour. The reaction mixture was diluted with brine (10 mL) and extracted with ethyl acetate (3×20 mL). The combined organic layers were dried over sodium sulfate, filtered, and concentrated under reduced pressure to obtain compound 11, which was used in the next step without further purification.

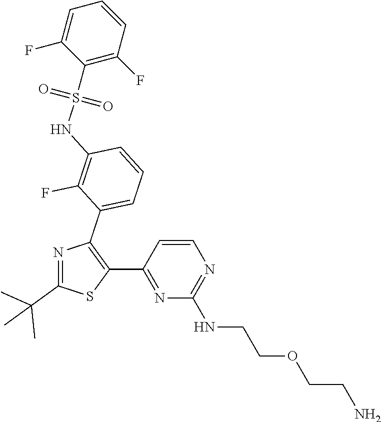 [SMILES: CC(C)(C)c1nc(-c2cccc(NS(=O)(=O)c3c(F)cccc3F)c2F)c(-c2ccnc(NCCOCCN)n2)s1]

### N-(3-(5-(2-((2-(2-aminoethoxy)ethyl)amino)pyrimidin-4-yl)-2-(tert-butyl)thiazol-4-yl)-2-fluorophenyl)-2,6-difluorobenzenesulfonamide (12)

A solution of compound 11 in DCM (2 mL) and TFA (1 mL) was stirred at RT for 1 hour. The reaction mixture was concentrated under reduced pressure, and purified by HPLC to afford the compound 12 (76 mmol, 0.105 mmol, 66% over 2 steps).

LC/MS (ESI) m/z: 608.0 [M+H]⁺.

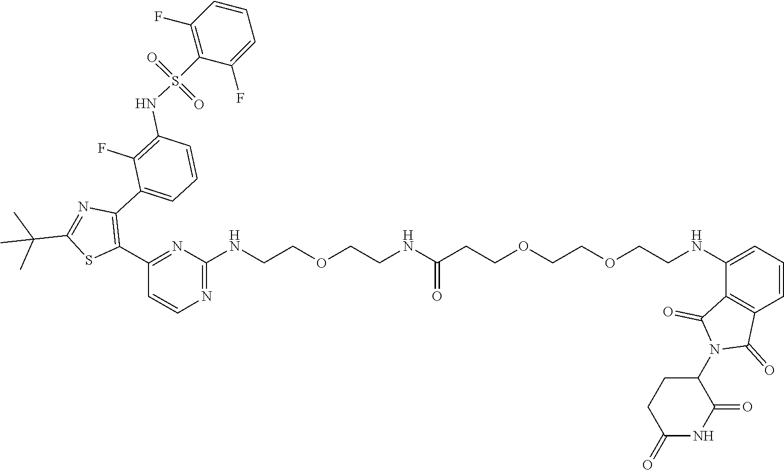 [SMILES: CC(C)(C)c1nc(-c2cccc(NS(=O)(=O)c3c(F)cccc3F)c2F)c(-c2ccnc(NCCOCCNC(=O)CCOCCOCCNc3cccc4c3C(=O)N(C3CCC(=O)NC3=O)C4=O)n2)s1]

### DD-03-106-1

A mixture of compound 12 (36 mg, 0.05 mmol), compound 4 (20 mg, 0.05 mmol), HATU (25 mg, 0.065 mmol), and DIPEA (26 μL, 0.15 mmol) in DMF (1 mL) was stirred at RT for 30 minutes. The reaction mixture was purified by HPLC to afford DD-03-106-1 (14 mg, 0.013 mmol, 26%) as a yellow solid.

¹H NMR (500 MHz, MeOD) δ 8.05 (d, J=6.3 Hz, 1H), 7.62 (tt, J=8.4, 5.9 Hz, 1H), 7.56-7.48 (m, 2H), 7.39 (ddd, J=7.9, 6.2, 1.7 Hz, 1H), 7.29 (t, J=7.9 Hz, 1H), 7.15-7.07 (m, 2H), 7.07-6.98 (m, 2H), 5.05 (dd, J=12.7, 5.5 Hz, 1H), 3.73 (dt, J=20.3, 5.6 Hz, 4H), 3.66-3.62 (m, 4H), 3.55 (t, J=5.4 Hz, 2H), 3.46 (t, J=5.2 Hz, 2H), 3.37 (t, J=5.4 Hz, 2H), 2.86 (ddd, J=17.5, 13.9, 5.3 Hz, 1H), 2.79-2.64 (m, 2H), 2.46 (t, J=6.0 Hz, 2H), 2.10 (dtd, J=13.1, 5.6, 2.8 Hz, 1H), 1.49 (s, 9H).

LC/MS (ESI) m/z: 1023.2 [M+H]⁺.

### Example 30: Synthesis of N-(2-(2-((4-(2-(tert-butyl)-4-(3-((2,6-difluorophenyl)sulfonamido)-2-fluorophenyl)thiazol-5-yl)pyrimidin-2-yl)amino)ethoxy)ethyl)-3-(2-(2-((2-(2,6-dioxopiperidin-3-yl)-1-oxoisoindolin-4-yl)amino)ethoxy)ethoxy)propenamide (DD-03-107-1)

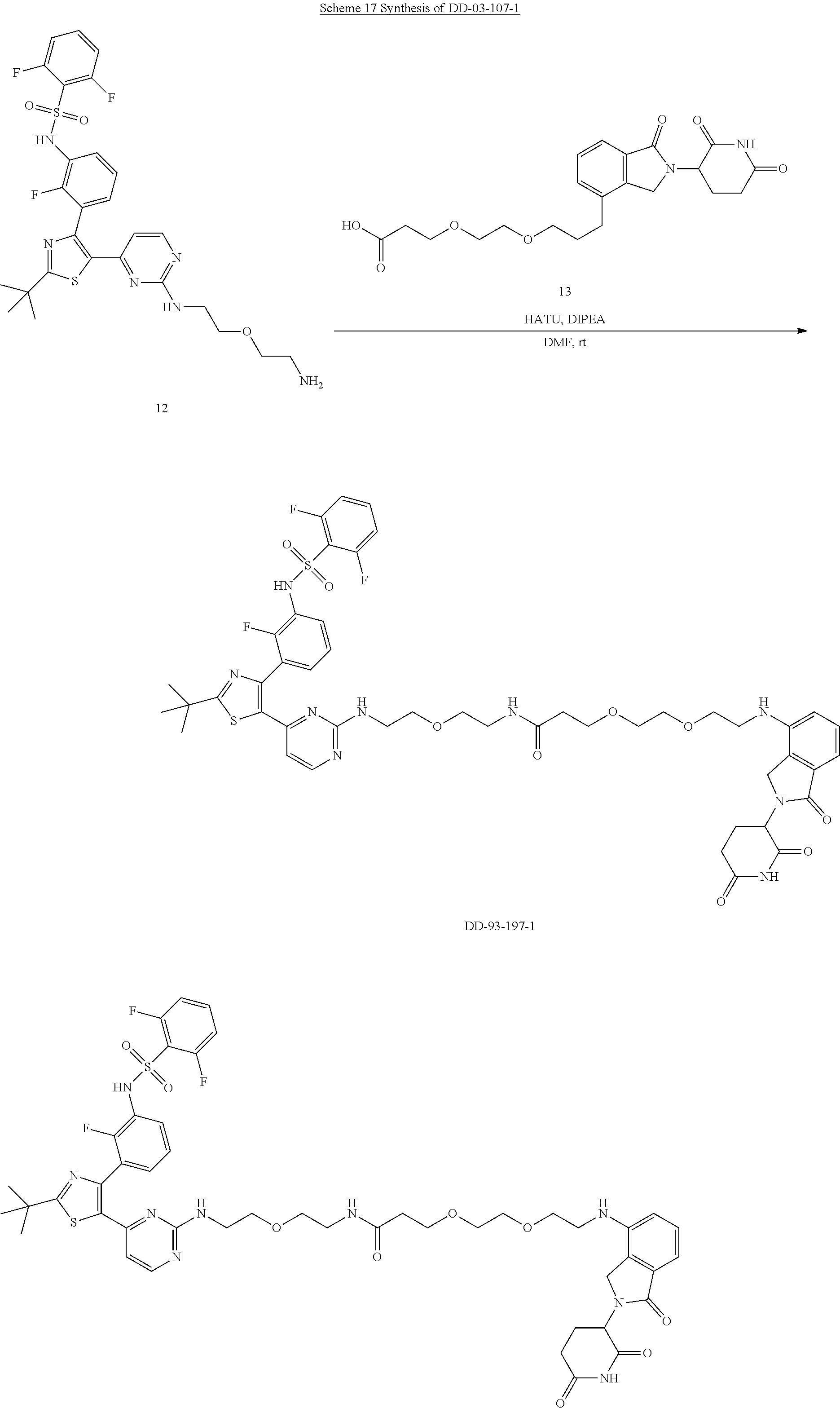 [SMILES: CC(C)(C)c1nc(-c2cccc(NS(=O)(=O)c3c(F)cccc3F)c2F)c(-c2ccnc(NCCOCCN)n2)s1 ; O=C(O)CCOCCOCCCc1cccc2c1CN(C1CCC(=O)NC1=O)C2=O ; CC(C)(C)c1nc(-c2cccc(NS(=O)(=O)c3c(F)cccc3F)c2F)c(-c2ccnc(NCCOCCNC(=O)CCOCCOCCNc3cccc4c3CN(C3CCC(=O)NC3=O)C4=O)n2)s1 ; CC(C)(C)c1nc(-c2cccc(NS(=O)(=O)c3c(F)cccc3F)c2F)c(-c2ccnc(NCCOCCNC(=O)CCOCCOCCNc3cccc4c3CN(C3CCC(=O)NC3=O)C4=O)n2)s1]

### DD-03-107-1

A mixture of compound 12 (31 mg, 0.043 mmol), compound 13 (18 mg, 0.043 mmol), HATU (21 mg, 0.056 mmol), and DIPEA (22 μL, 0.13 mmol) in DMF (1 mL) was stirred at RT for 1 hour. The reaction mixture was purified by HPLC to afford DD-03-107-1 (20 mg, 0.018 mmol, 41%).

¹H NMR (500 MHz, MeOD) δ 8.05 (s, 1H), 7.62 (tt, J=8.5, 5.9 Hz, 1H), 7.52 (td. J=7.7, 1.7 Hz, 1H), 7.44-7.37 (m, 1H), 7.30 (q, J=8.1 Hz, 3H), 7.11 (ddd, J=11.7, 7.9, 2.3 Hz, 4H), 6.87 (s, 1H), 6.45 (s, 2H), 5.17 (dd, J=13.4, 5.2 Hz, 1H), 4.39-4.25 (m, 2H), 3.72 (dt, J=18.0, 5.8 Hz, 4H), 3.63 (q, J=1.5 Hz, 3H), 3.52 (t, J=5.5 Hz, 2H), 3.43-3.34 (m, 3H), 2.92 (ddd, J=17.5, 13.5, 5.4 Hz, 1H), 2.80 (ddd, J=17.6, 4.6, 2.4 Hz, 1H), 2.55-2.41 (m, 3H), 2.18 (dtd, J=12.9, 5.3, 2.4 Hz, 1H), 1.50 (s, 9H).

LC/MS (ESI) m/z: 1009.1 [M+H]⁺.

### Example 31: Synthesis of (2S,4R)-1-((S)-2-(3-(2-(2-((4-(2-(tert-butyl)-4-(3-((2,6-difluorophenyl)sulfonamido)-2-fluorophenyl)thiazol-5-yl)pyrimidin-2-yl)amino)ethoxy)ethoxy)propanamido)-3,3-dimethylbutanoyl)-4-hydroxy-N—((S)-1-(4-(4-methylthiazol-5-yl)phenyl)ethyl)pyrrolidine-2-2377 carboxamide (DD-03-156-1)

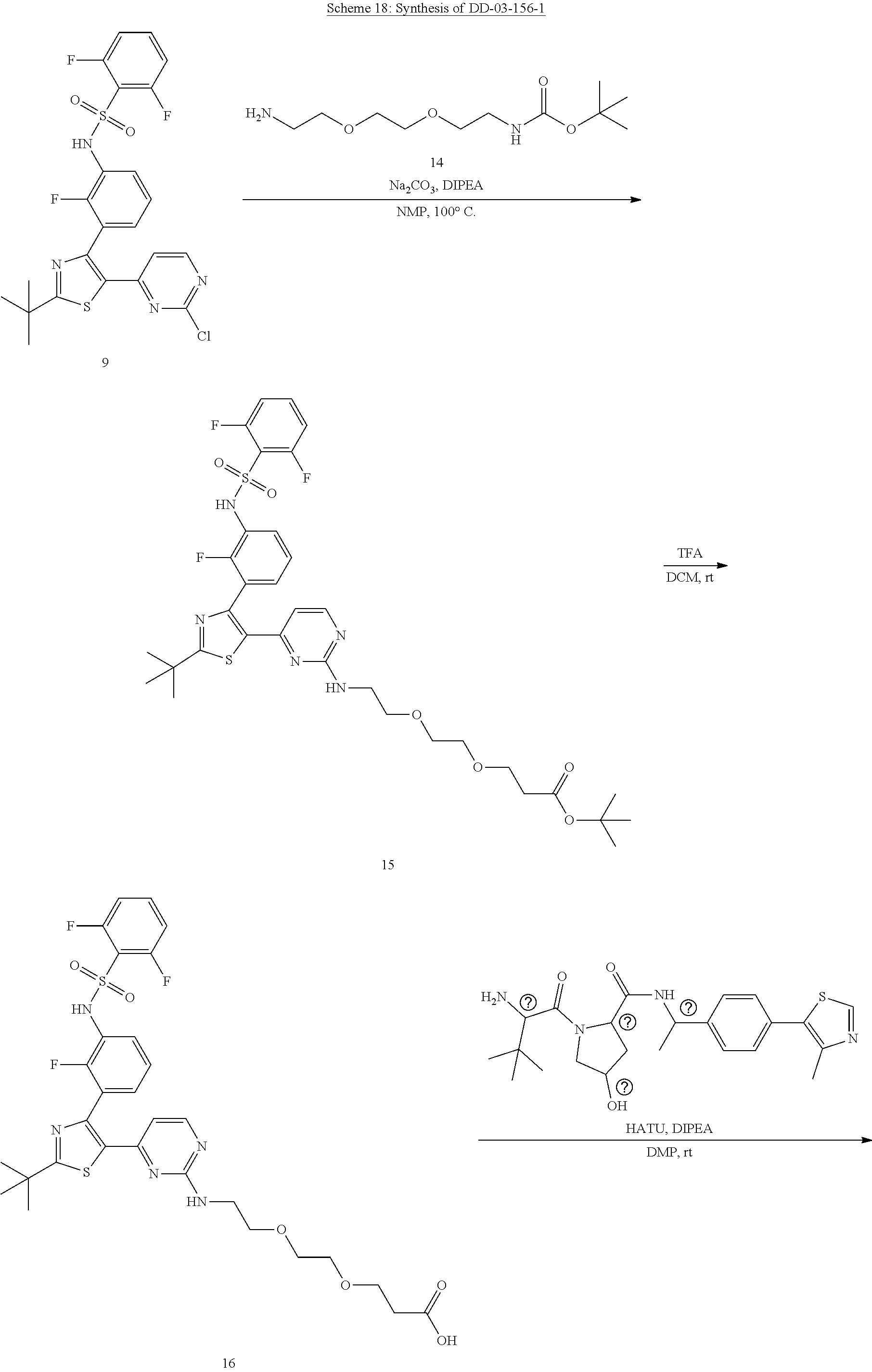 [SMILES: CC(C)(C)c1nc(-c2cccc(NS(=O)(=O)c3c(F)cccc3F)c2F)c(-c2ccnc(Cl)n2)s1 ; CC(C)(C)OC(=O)NCCOCCOCCN ; CC(C)(C)OC(=O)CCOCCOCCNc1nccc(-c2sc(C(C)(C)C)nc2-c2cccc(NS(=O)(=O)c3c(F)cccc3F)c2F)n1 ; CC(C)(C)c1nc(-c2cccc(NS(=O)(=O)c3c(F)cccc3F)c2F)c(-c2ccnc(NCCOCCOCCC(=O)O)n2)s1 ; Cc1ncsc1-c1ccc(C(C)NC(=O)C2CC(O)CN2C(=O)C(N)C(C)(C)C)cc1]

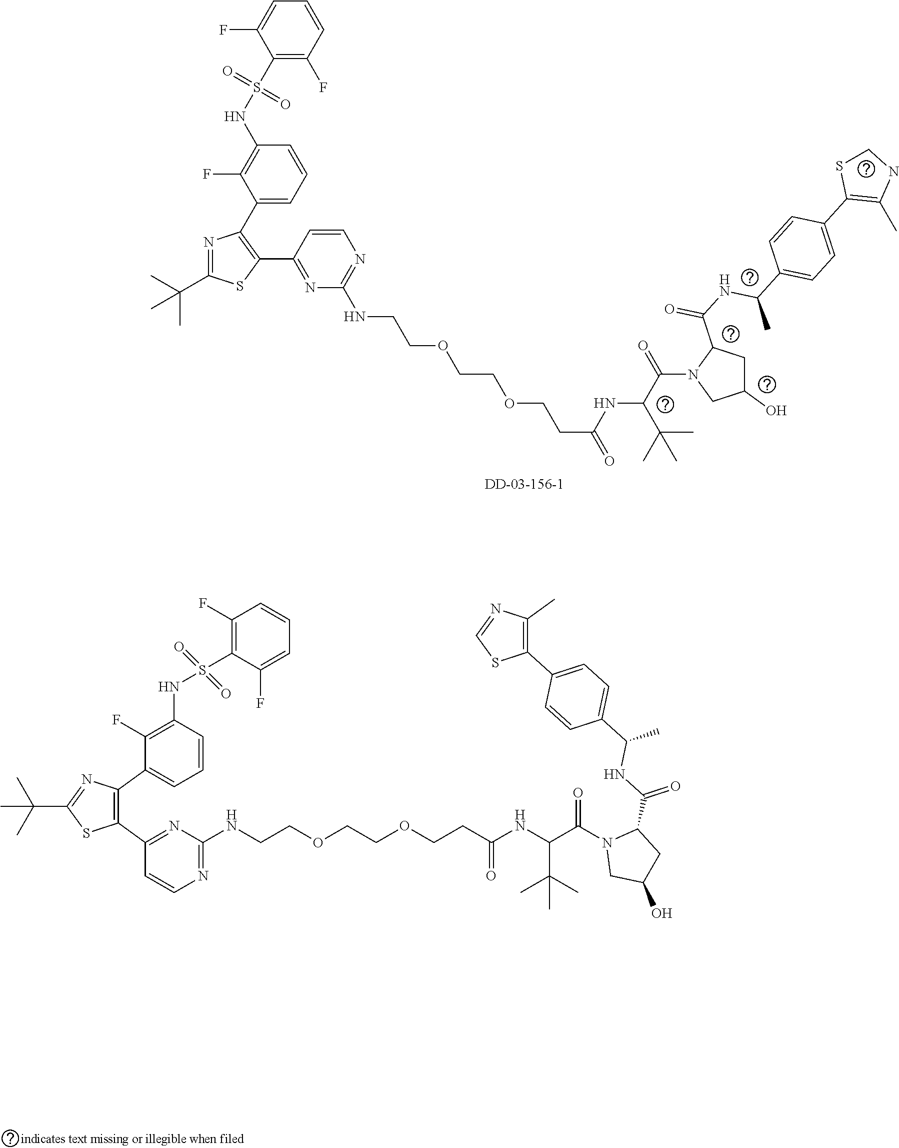

### DD-03-156-1

A mixture of compound 16 (36 mg, 0.05 mmol), VHL-amine (22 mg, 0.05 mmol), HATU (25 mg, 0.065 mmol), and DIPEA (26 μL, 0.15 mmol) in DMF (1 mL) was stirred at RT for 1 hour. The reaction mixture was purified by HPLC to afford DD-03-156-1 (5 mg, 0.004 mmol, 8%).

¹H NMR (500 MHz, MeOD) δ 8.92 (d, J=3.2 Hz, 1H), 8.05 (d, J=6.1 Hz, 1H), 7.63 (ddd, J=15.9, 9.4, 6.8 Hz, 2H), 7.57-7.50 (m, 1H), 7.43 (dd, J=9.0, 6.7 Hz, 4H), 7.35-7.26 (m, 1H), 7.11 (td, J=9.0, 4.8 Hz, 3H), 6.39 (s, 1H), 5.00 (q, J=7.0 Hz, 1H), 4.68 (s, 1H), 4.63-4.56 (m, 1H), 4.45 (s, 1H), 3.89 (d, J=11.1 Hz, 1H), 3.82-3.70 (mi, 4H), 3.66 (td, J=5.5, 3.4 Hz, 8H), 3.52 (s, 4H), 2.68 (s, 1H), 2.64-2.50 (m, 1H), 2.48 (d, J=1.8 Hz, 4H), 2.22 (dd, J=13.3, 7.8 Hz, 1H), 1.99 (ddd, J=13.4, 9.2, 4.5 Hz, 1H), 1.50 (s, 9H), 1.06 (s, 9H).

LC/MS (ESI) m/z: 1107.4[M+H]⁺.

All patent publications and non-patent publications are indicative of the level of skill of those skilled in the art to which this invention pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

APPENDIX II

| Freq | Freq.down | Freq.up | ID | Accession | Experiment.ID | Compound | direction.of.change | logFC | P.Value | Accession.trimmed |
|---|---|---|---|---|---|---|---|---|---|---|
| 60 | 60 | 0 | RPS6KA1 | Q15418-2 | wp012_TI13.112 | TI13.112 | down | −0.78754201 | 6.63E−08 | Q15418 |
| 60 | 60 | 0 | RPS6KA1 | Q15418-2 | wp016_SK.3.91_1 uM | SK.3.91 | down | −0.41458581 | 0.00080193 | Q15418 |
| 60 | 60 | 0 | RPS6KA1 | Q15418-2 | wp016_SK.3.93 | SK.3.93 | down | −0.34133234 | 0.00177903 | Q15418 |
| 60 | 60 | 0 | RPS6KA1 | Q15418-2 | wp076_MFH.5.103.1 | MFH.5.103.1 | down | −1.58284701 | 9.87E−07 | Q15418 |
| 60 | 60 | 0 | RPS6KA1 | Q15418-2 | wp076_MFH.5.116.1 | MFH.5.116.1 | down | −0.57573651 | 8.40E−05 | Q15418 |
| 60 | 60 | 0 | RPS6KA1 | Q15418-2 | wp133_SK.3.91_4 hr | SK.3.91 | down | −0.35983267 | 5.27E−06 | Q15418 |
| 60 | 60 | 0 | RPS6KA1 | Q15418-2 | wp137_TL12.186 | TL12.186 | down | −0.45136365 | 0.0001293 | Q15418 |
| 60 | 60 | 0 | RPS6KA1 | Q15418-2 | wp137_TL13.97 | TL13.97 | down | −0.37573195 | 0.00033813 | Q15418 |
| 60 | 60 | 0 | RPS6KA1 | Q15418-2 | wp146_TMX.01.160 | TMX.01.160 | down | −0.43167866 | 0.00026205 | Q15418 |
| 60 | 60 | 0 | RPS6KA1 | Q15418-2 | wp146_TMX.02.138 | TMX.02.138 | down | −0.500997 | 0.00015288 | Q15418 |
| 60 | 60 | 0 | RPS6KA1 | Q15418-2 | wp146_TMX.02.172 | TMX.02.172 | down | −1.35051368 | 4.10E−06 | Q15418 |
| 60 | 60 | 0 | RPS6KA1 | Q15418-2 | wp146_TMX.02.174 | TMX.02.174 | down | −0.6638377 | 5.50E−05 | Q15418 |
| 60 | 60 | 0 | RPS6KA1 | Q15418-1 | wp107_SK.3.91 | SK.3.91 | down | −0.36714818 | 1.33E−05 | Q15418 |
| 60 | 60 | 0 | RPS6KA1 | Q15418-1 | wp131_TL12.186 | TL12.186 | down | −0.35621124 | 4.61E−06 | Q15418 |
| 60 | 60 | 0 | RPS6KA1 | Q15418-1 | wp160_DB0646_8 hr | DB0646 | down | −0.41297764 | 0.00020844 | Q15418 |
| 60 | 60 | 0 | RPS6KA1 | Q15418-1 | wp160_SK.3.91_4 hr | SK.3.91 | down | −0.36298311 | 0.00034762 | Q15418 |
| 60 | 60 | 0 | RPS6KA1 | Q15418-1 | wp168_DB0662 | DB0662 | down | −0.47241575 | 1.54E−05 | Q15418 |
| 60 | 60 | 0 | RPS6KA1 | Q15418-1 | wp168_DB1113 | DB1113 | down | −0.72972324 | 2.91E−07 | Q15418 |
| 60 | 60 | 0 | RPS6KA1 | Q15418-1 | wp172_SK.3.91 | SK.3.91 | down | −0.45610982 | 4.97E−05 | Q15418 |
| 60 | 60 | 0 | RPS6KA1 | Q15418 | wp152_DB1114 | DB1114 | down | −0.3962409 | 2.44E−06 | Q15418 |
| 59 | 58 | 1 | CDK4 | P11802 | wp005_BSJ.01.147 | BSJ.01.147 | down | −1.41499961 | 6.50E−06 | P11802 |
| 59 | 58 | 1 | CDK4 | P11802 | wp005_YKL.6.102 | YKL.6.102 | down | −0.64195122 | 5.95E−06 | P11802 |
| 59 | 58 | 1 | CDK4 | P11802 | wp016_LT2.49 | LT2.49 | down | −0.89850063 | 0.00087723 | P11802 |
| 59 | 58 | 1 | CDK4 | P11802 | wp016_SK.3.89 | SK.3.89 | down | −0.51770953 | 0.00781658 | P11802 |
| 59 | 58 | 1 | CDK4 | P11802 | wp016_SK.3.91 | SK.3.91 | down | −1.160981 | 0.00030027 | P11802 |
| 59 | 58 | 1 | CDK4 | P11802 | wp016_SK.3.91_1 uM | SK.3.91 | down | −1.42853107 | 0.00012439 | P11802 |
| 59 | 58 | 1 | CDK4 | P11802 | wp016_SK.3.93 | SK.3.93 | down | −1.30450325 | 0.0001832 | P11802 |
| 59 | 58 | 1 | CDK4 | P11802 | wp016_TL12.186 | TL12.186 | down | −0.91661043 | 0.00080768 | P11802 |
| 59 | 58 | 1 | CDK4 | P11802 | wp045_DB0614 | DB0614 | down | −1.53939819 | 3.93E−06 | P11802 |
| 59 | 58 | 1 | CDK4 | P11802 | wp045_DB0646 | DB0646 | down | −0.90115183 | 4.07E−05 | P11802 |
| 59 | 58 | 1 | CDK4 | P11802 | wp045_DB0663 | DB0663 | down | −0.68226285 | 0.00013572 | P11802 |
| 59 | 58 | 1 | CDK4 | P11802 | wp045_RSS0628 | RSS0628 | down | −1.42232907 | 5.56E−06 | P11802 |
| 59 | 58 | 1 | CDK4 | P11802 | wp047_DD.03.106 | DD.03.106 | down | −0.92772254 | 4.93E−05 | P11802 |
| 59 | 58 | 1 | CDK4 | P11802 | wp076_MFH.5.115.2 | MFH.5.115.2 | down | −0.72460515 | 0.00045685 | P11802 |
| 59 | 58 | 1 | CDK4 | P11802 | wp076_MFH.5.126.1 | MFH.5.126.1 | down | −0.60797486 | 0.00095692 | P11802 |
| 59 | 58 | 1 | CDK4 | P11802 | wp080_RSS0680 | RSS0680 | down | −1.72443219 | 7.44E−07 | P11802 |
| 59 | 58 | 1 | CDK4 | P11802 | wp081_TL13.178 | TL13.178 | down | −0.57620467 | 1.18E−05 | P11802 |
| 59 | 58 | 1 | CDK4 | P11802 | wp084_BSJ.01.147 | BSJ.01.147 | down | −1.80038116 | 2.20E−08 | P11802 |
| 59 | 58 | 1 | CDK4 | P11802 | wp084_BSJ.03.123 | BSJ.03.123 | down | −0.9496023 | 4.75E−07 | P11802 |
| 59 | 58 | 1 | CDK4 | P11802 | wp084_BSJ.03.204 | BSJ.03.204 | down | −2.16193918 | 4.35E−08 | P11802 |
| 59 | 58 | 1 | CDK4 | P11802 | wp084_BSJ.04.132 | BSJ.04.132 | down | −0.90461352 | 8.29E−07 | P11802 |
| 59 | 58 | 1 | CDK4 | P11802 | wp103_DB0646 | DB0646 | down | −0.5014826 | 0.00059779 | P11802 |
| 59 | 58 | 1 | CDK4 | P11802 | wp103_SK.3.91 | SK.3.91 | down | −1.0291791 | 2.40E−05 | P11802 |
| 59 | 58 | 1 | CDK4 | P11802 | wp104_TMX.01.160.1_0.25 | TMX.01.160.1 | down | −0.55415438 | 0.00014093 | P11802 |
| 59 | 58 | 1 | CDK4 | P11802 | wp104_TMX.01.160.1_1 | TMX.01.160.1 | down | −0.76762278 | 2.78E−05 | P11802 |
| 59 | 58 | 1 | CDK4 | P11802 | wp105_SK.3.91 | SK.3.91 | down | −0.79948385 | 1.52E−06 | P11802 |
| 59 | 58 | 1 | CDK4 | P11802 | wp107_DB0646 | DB0646 | down | −0.55652489 | 9.79E−05 | P11802 |
| 59 | 58 | 1 | CDK4 | P11802 | wp107_SK.3.91 | SK.3.91 | down | −1.27241766 | 3.97E−06 | P11802 |
| 59 | 58 | 1 | CDK4 | P11802 | wp108_FMF.06.098.1 | FMF.06.098.1 | down | −0.87305234 | 1.33E−05 | P11802 |
| 59 | 58 | 1 | CDK4 | P11802 | wp114_FMF.06.098.1 | FMF.06.098.1 | down | −0.37811659 | 0.0027291 | P11802 |
| 59 | 58 | 1 | CDK4 | P11802 | wp114_SK.3.91 | SK.3.91 | down | −0.56050036 | 0.00059624 | P11802 |
| 59 | 58 | 1 | CDK4 | P11802 | wp117_DB0614 | DB0614 | down | −1.53852534 | 1.84E−07 | P11802 |
| 59 | 58 | 1 | CDK4 | P11802 | wp117_DB0646 | DB0646 | down | −0.667307 | 7.44E−06 | P11802 |
| 59 | 58 | 1 | CDK4 | P11802 | wp117_RSS0680 | RSS0680 | down | −1.6785872 | 2.84E−06 | P11802 |
| 59 | 58 | 1 | CDK4 | P11802 | wp118_INY.03.041.01 | INY.03.041.01 | down | −0.40187929 | 0.00011409 | P11802 |
| 59 | 58 | 1 | CDK4 | P11802 | wp131_FMF.06.098.1 | FMF.06.098.1 | down | −1.0288969 | 6.67E−06 | P11802 |
| 59 | 58 | 1 | CDK4 | P11802 | wp131_TL12.186 | TL12.186 | down | −1.18604366 | 3.88E−07 | P11802 |
| 59 | 58 | 1 | CDK4 | P11802 | wp133_SK.3.91_2 hr | SK.3.91 | down | −0.7483541 | 1.50E−07 | P11802 |
| 59 | 58 | 1 | CDK4 | P11802 | wp133_SK.3.91_4 hr | SK.3.91 | down | −1.29170378 | 3.08E−09 | P11802 |
| 59 | 58 | 1 | CDK4 | P11802 | wp135_INY.03.041_01_8 hr | INY.03.041_01 | down | −0.32813992 | 0.00018151 | P11802 |
| 59 | 58 | 1 | CDK4 | P11802 | wp137_BS.J.03.204 | BSJ.03.204 | down | −2.11771665 | 3.45E−09 | P11802 |
| 59 | 58 | 1 | CDK4 | P11802 | wp137_DD.03.106 | DD.03.106 | down | −0.88854976 | 4.37E−07 | P11802 |
| 59 | 58 | 1 | CDK4 | P11802 | wp137_TL12.186 | TL12.186 | down | −1.1726108 | 9.33E−08 | P11802 |
| 59 | 58 | 1 | CDK4 | P11802 | wp137_TL13.97 | TL13.97 | down | −0.96756031 | 2.72E−07 | P11802 |
| 59 | 58 | 1 | CDK4 | P11802 | wp147_DD.03.106_2 uM | DD.03.106 | down | −0.98354041 | 2.73E−07 | P11802 |
| 59 | 58 | 1 | CDK4 | P11802 | wp147_DD.03.106_1 uM | DD.03.106 | down | −0.69608249 | 1.61E−06 | P11802 |
| 59 | 58 | 1 | CDK4 | P11802 | wp147_DD.03.106_4 uM | DD.03.106 | down | −0.726042 | 1.30E−06 | P11802 |
| 59 | 58 | 1 | CDK4 | P11802 | wp152_DB0646 | DB0646 | down | −0.73603754 | 1.02E−05 | P11802 |
| 59 | 58 | 1 | CDK4 | P11802 | wp152_DB1114 | DB1114 | down | −0.67230936 | 1.66E−05 | P11802 |
| 59 | 58 | 1 | CDK4 | P11802 | wp152_DB118430 | DB118430 | down | −0.36443571 | 0.00042228 | P11802 |
| 59 | 58 | 1 | CDK4 | P11802 | wp160_DB0646_4 hr | DB0646 | down | −0.72625738 | 0.00011267 | P11802 |
| 59 | 58 | 1 | CDK4 | P11802 | wp160_DB0646_8 hr | DB0646 | down | −1.11387829 | 2.02E−05 | P11802 |
| 59 | 58 | 1 | CDK4 | P11802 | wp160_SK.3.91_1 hr | SK.3.91 | down | −0.43287734 | 0.00087089 | P11802 |
| 59 | 58 | 1 | CDK4 | P11802 | wp160_SK.3.91_2 hr | SK.3.91 | down | −0.91037873 | 4.55E−05 | P11802 |
| 59 | 58 | 1 | CDK4 | P11802 | wp160_SK.3.91_4 hr | SK.3.91 | down | −1.35310233 | 9.21E−06 | P11802 |
| 59 | 58 | 1 | CDK4 | P11802 | wp168_DB0661 | DB0661 | down | −0.49778163 | 3.38E−05 | P11802 |
| 59 | 58 | 1 | CDK4 | P11802 | wp168_DB0662 | DB0662 | down | −0.98831986 | 4.71E−07 | P11802 |
| 59 | 58 | 1 | CDK4 | P11802 | wp168_DB1113 | DB1113 | down | −0.43731926 | 0.00012979 | P11802 |
| 56 | 53 | 3 | AURKA | O14965 | wp011_TL13.12 | TL13.12 | down | −0.59880532 | 5.58E−05 | O14965 |
| 56 | 53 | 3 | AURKA | O14965 | wp014_TL13.97 | TL13.97 | down | −0.32682475 | 0.00018426 | O14965 |
| 56 | 53 | 3 | AURKA | O14965 | wp016_LT2.49 | LT2.49 | down | −1.85342307 | 0.00030571 | O14965 |
| 56 | 53 | 3 | AURKA | O14965 | wp016_SK.3.87 | SK.3.87 | down | −1.38130869 | 0.00104301 | O14965 |
| 56 | 53 | 3 | AURKA | O14965 | wp016_SK.3.89 | SK.3.89 | down | −1.67215622 | 0.00047141 | O14965 |
| 56 | 53 | 3 | AURKA | O14965 | wp016_SK.3.91 | SK.3.91 | down | −1.90797167 | 0.00027043 | O14965 |
| 56 | 53 | 3 | AURKA | O14965 | wp016_SK.3.91_1 uM | SK.3.91 | down | −3.02404677 | 3.77E−05 | O14965 |
| 56 | 53 | 3 | AURKA | O14965 | wp016_SK.3.93 | SK.3.93 | down | −1.82311284 | 0.00032774 | O14965 |
| 56 | 53 | 3 | AURKA | O14965 | wp016_TL12.186 | TL12.186 | down | −1.7584579 | 0.0003816 | O14965 |
| 56 | 53 | 3 | AURKA | O14965 | wp045_DB0614 | DB0614 | down | −0.38504211 | 0.0050025 | O14965 |
| 56 | 53 | 3 | AURKA | O14965 | wp045_DB0646 | DB0646 | down | −0.32416571 | 0.00961326 | O14965 |
| 56 | 53 | 3 | AURKA | O14965 | wp045_RSS0628 | RSS0628 | down | −1.13039581 | 5.57E−05 | O14965 |
| 56 | 53 | 3 | AURKA | O14965 | wp056_SB1.G.190 | SB1.G.190 | down | −0.84298794 | 0.00054387 | O14965 |
| 56 | 53 | 3 | AURKA | O14965 | wp071_JWG.118 | JWG.118 | down | −0.88607338 | 6.29E−05 | O14965 |
| 56 | 53 | 3 | AURKA | O14965 | wp071_SB1.G.181 | SB1.G.181 | down | −0.36844649 | 0.00354434 | O14965 |
| 56 | 53 | 3 | AURKA | O14965 | wp071_SB1.G.192.1 | SB1.G.192.1 | down | −0.8111076 | 9.60E−05 | O14965 |
| 56 | 53 | 3 | AURKA | O14965 | wp078_MFH.5.103.1 | MFH.5.103.1 | down | −0.98925963 | 0.00023023 | O14965 |
| 56 | 53 | 3 | AURKA | O14965 | wp076_MFH.5.115.2 | MFH.5.115.2 | down | −0.59121714 | 0.00200241 | O14965 |
| 56 | 53 | 3 | AURKA | O14965 | wp076_MFH.5.116.1 | MFH.5.116.1 | down | −0.70699871 | 0.00095711 | O14965 |
| 56 | 53 | 3 | AURKA | O14965 | wp076_MFH.5.126.1 | MFH.5.126.1 | down | −0.94742097 | 0.00027712 | O14965 |
| 56 | 53 | 3 | AURKA | O14965 | wp080_dAURK.4 | dAURK.4 | down | −1.05515983 | 4.40E−05 | O14965 |
| 56 | 53 | 3 | AURKA | O14965 | wp081_SB1.G.192.1 | SB1.G.192.1 | down | −0.72992079 | 3.83E−06 | O14965 |
| 56 | 53 | 3 | AURKA | O14965 | wp088_SB1.G.194 | SB1.G.194 | down | −1.17476088 | 0.00017375 | O14965 |
| 56 | 53 | 3 | AURKA | O14965 | wp103_SK.3.91 | SK.3.91 | down | −1.58676417 | 7.94E−07 | O14965 |
| 56 | 53 | 3 | AURKA | O14965 | wp104_TMX.01.160.1_0.25 | TMX.01.160.1 | down | −1.32589599 | 1.31E−06 | O14965 |
| 56 | 53 | 3 | AURKA | O14965 | wp104_TMX.01.160.1_1 | TMX.01.160.1 | down | −1.46623421 | 7.85E−07 | O14965 |
| 56 | 53 | 3 | AURKA | O14965 | wp105_SK.3.91 | SK.3.91 | down | −1.83717296 | 3.58E−08 | O14965 |
| 56 | 53 | 3 | AURKA | O14965 | wp107_SK.3.91 | SK.3.91 | down | −3.06883023 | 2.92E−08 | O14965 |
| 56 | 53 | 3 | AURKA | O14965 | wp114_FMF.06.098.1 | FMF.06.098.1 | down | −0.41507448 | 0.00382842 | O14965 |
| 56 | 53 | 3 | AURKA | O14965 | wp114_SK.3.91 | SK.3.91 | down | −1.22761574 | 5.42E−05 | O14965 |
| 56 | 53 | 3 | AURKA | O14965 | wp117_DB0614 | DB0614 | down | −0.45473918 | 3.25E−05 | O14965 |
| 56 | 53 | 3 | AURKA | O14965 | wp131_FMF.06.098.1 | FMF.06.098.1 | down | −0.34714997 | 3.11E−05 | O14965 |
| 56 | 53 | 3 | AURKA | O14965 | wp131_SB1.G.192.1 | SB1.G.192.1 | down | −0.6267162 | 1.52E−05 | O14965 |
| 56 | 53 | 3 | AURKA | O14965 | wp131_TL12.186 | TL12.186 | down | −3.19296464 | 1.94E−07 | O14965 |
| 56 | 53 | 3 | AURKA | O14965 | wp133_SK.3.91_1 hr | SK.3.91 | down | −1.32854162 | 1.69E−08 | O14965 |
| 56 | 53 | 3 | AURKA | O14965 | wp133_SK.3.91_2 hr | SK.3.91 | down | −2.09229652 | 4.85E−10 | O14965 |
| 56 | 53 | 3 | AURKA | O14965 | wp133_SK.3.91_4 hr | SK.3.91 | down | −3.74462909 | 3.74E−10 | O14965 |
| 56 | 53 | 3 | AURKA | O14965 | wp134_BSJ.04.178 | BSJ.04.178 | down | −0.47723869 | 0.00011045 | O14965 |
| 56 | 53 | 3 | AURKA | O14965 | wp134_JWG.120 | JWG.120 | down | −1.56165816 | 1.30E−07 | O14965 |
| 56 | 53 | 3 | AURKA | O14965 | wp137_TL12.186 | TL12.186 | down | −2.64216851 | 5.67E−09 | O14965 |
| 56 | 53 | 3 | AURKA | O14965 | wp137_TL13.97 | TL13.97 | down | −1.61190283 | 8.93E−08 | O14965 |
| 56 | 53 | 3 | AURKA | O14965 | wp146_TMX.01.160 | TMX.01.160 | down | −2.62119184 | 7.78E−05 | O14965 |
| 56 | 53 | 3 | AURKA | O14965 | wp146_TMX.02.138 | TMX.02.138 | down | −2.93269494 | 5.17E−05 | O14965 |
| 56 | 53 | 3 | AURKA | O14965 | wp146_TMX.02.172 | TMX.02.172 | down | −2.40199336 | 0.00010688 | O14965 |
| 56 | 53 | 3 | AURKA | O14965 | wp146_TMX.02.174 | TMX.02.174 | down | −2.86510208 | 5.63E−05 | O14965 |
| 56 | 53 | 3 | AURKA | O14965 | wp147_DD.03.106_4 uM | DD.03.106 | down | −0.33022961 | 0.00414912 | O14965 |
| 56 | 53 | 3 | AURKA | O14965 | wp160_DB0646_4 hr | DB0646 | down | −0.44779336 | 0.00736614 | O14965 |
| 56 | 53 | 3 | AURKA | O14965 | wp160_SK.3.91_1 hr | SK.3.91 | down | −0.54695436 | 0.00354671 | O14965 |
| 56 | 53 | 3 | AURKA | O14965 | wp160_SK.3.91_2 hr | SK.3.91 | down | −0.84563829 | 0.00066907 | O14965 |
| 56 | 53 | 3 | AURKA | O14965 | wp160_SK.3.91_4 hr | SK.3.91 | down | −1.39096593 | 9.31E−05 | O14965 |
| 56 | 53 | 3 | AURKA | O14965 | wp164_SB1.G.194 | SB1.G.194 | down | −2.03482025 | 6.15E−07 | O14965 |
| 56 | 53 | 3 | AURKA | O14965 | wp172_SK.3.91 | SK.3.91 | down | −2.0476493 | 3.29E−07 | O14965 |
| 56 | 53 | 3 | AURKA | O14965 | wp195_BJG.04.203 | BJG.04.203 | down | −0.43756832 | 5.74E−05 | O14965 |
| 52 | 52 | 0 | WEE1 | P30291-1 | wp005_BSJ.01.147 | BSJ.01.147 | down | −0.44696792 | 0.00093643 | P30291 |
| 52 | 52 | 0 | WEE1 | P30291-1 | wp005_YKL.6.102 | YKL.6.102 | down | −0.36487588 | 3.43E−05 | P30291 |
| 52 | 52 | 0 | WEE1 | P30291-1 | wp016_LT2.49 | LT2.49 | down | −1.31437542 | 2.98E−05 | P30291 |
| 52 | 52 | 0 | WEE1 | P30291-1 | wp016_SK.3.87 | SK.3.87 | down | −0.92731816 | 0.00013353 | P30291 |
| 52 | 52 | 0 | WEE1 | P30291-1 | wp016_SK.3.89 | SK.3.89 | down | −1.01700699 | 9.00E−05 | P30291 |
| 52 | 52 | 0 | WEE1 | P30291-1 | wp016_SK.3.91 | SK.3.91 | down | −1.27769936 | 3.37E−05 | P30291 |
| 52 | 52 | 0 | WEE1 | P30291-1 | wp016_SK.3.91_1 uM | SK.3.91 | down | −2.68401799 | 1.35E−06 | P30291 |
| 52 | 52 | 0 | WEE1 | P30291-1 | wp016_SK.3.93 | SK.3.93 | down | −1.17630187 | 4.82E−05 | P30291 |
| 52 | 52 | 0 | WEE1 | P30291-1 | wp016_TL12.186 | TL12.186 | down | −1.51432161 | 1.62E−05 | P30291 |
| 52 | 52 | 0 | WEE1 | P30291-1 | wp045_DB.3.291 | DB.3.291 | down | −0.41609948 | 0.00033806 | P30291 |
| 52 | 52 | 0 | WEE1 | P30291-1 | wp045_DB0614 | DB0614 | down | −1.87590851 | 4.80E−07 | P30291 |
| 52 | 52 | 0 | WEE1 | P30291-1 | wp045_RSS0628 | RSS0628 | down | −1.57814994 | 1.03E−06 | P30291 |
| 52 | 52 | 0 | WEE1 | P30291-1 | wp056_ZNL.02.096 | ZNL.02.096 | down | −2.40945157 | 1.50E−06 | P30291 |
| 52 | 52 | 0 | WEE1 | P30291-1 | wp071_JWG.118 | JWG.118 | down | −0.9953516 | 1.11E−05 | P30291 |
| 52 | 52 | 0 | WEE1 | P30291-1 | wp071_SB1.G.192.1 | SB1.G.192.1 | down | −2.36671244 | 1.61E−07 | P30291 |
| 52 | 52 | 0 | WEE1 | P30291-1 | wp076_MFH.5.103.1 | MFH.S.103.1 | down | −0.96937816 | 0.0004505 | P30291 |
| 52 | 52 | 0 | WEE1 | P30291-1 | wp076_MFH.5.115.2 | MFH.5.115.2 | down | −0.76642769 | 0.00120865 | P30291 |
| 52 | 52 | 0 | WEE1 | P30291-1 | wp076_MFH.5.116.1 | MFH.5.116.1 | down | −0.47991979 | 0.00780769 | P30291 |
| 52 | 52 | 0 | WEE1 | P30291-1 | wp076_MFH.5.126.1 | MFH.5.126.1 | down | −0.90476261 | 0.00060339 | P30291 |
| 52 | 52 | 0 | WEE1 | P30291-1 | wp080_RSS0680 | RSS0680 | down | −2.35874575 | 1.86E−08 | P30291 |
| 52 | 52 | 0 | WEE1 | P30291-1 | wp081_SB1.G.192.1 | SB1.G.192.1 | down | −1.76413476 | 1.51E−08 | P30291 |
| 52 | 52 | 0 | WEE1 | P30291-1 | wp081_ZNL.02.096 | ZNL.02.096 | down | −1.97002574 | 8.20E−09 | P30291 |
| 52 | 52 | 0 | WEE1 | P30291-1 | wp084_BSJ.01.147 | BSJ.01.147 | down | −0.5616837 | 4.38E−06 | P30291 |
| 52 | 52 | 0 | WEE1 | P30291-1 | wp084_BSJ.03.123 | BSJ.03.123 | down | −0.38830939 | 1.64E−05 | P30291 |
| 52 | 52 | 0 | WEE1 | P30291-1 | wp084_BSJ.03.204 | BSJ.03.204 | down | −0.5773583 | 2.77E−06 | P30291 |
| 52 | 52 | 0 | WEE1 | P30291-1 | wp103_SK.3.91 | SK.3.91 | down | −1.37115756 | 2.72E−06 | P30291 |
| 52 | 52 | 0 | WEE1 | P30291-1 | wp105_SK.3.91 | SK.3.91 | down | −1.22879423 | 4.64E−06 | P30291 |
| 52 | 52 | 0 | WEE1 | P30291-1 | wp106_ZNL.02.096_2 hr | ZNL.02.096 | down | −2.52491938 | 1.06E−12 | P30291 |
| 52 | 52 | 0 | WEE1 | P30291-1 | wp106_ZNL.02.096_4 hr | ZNL.02.096 | down | −2.76422364 | 1.83E−11 | P30291 |
| 52 | 52 | 0 | WEE1 | P30291-1 | wp107_SK.3.91 | SK.3.91 | down | −2.32910572 | 6.52E−10 | P30291 |
| 52 | 52 | 0 | WEE1 | P30291-1 | wp108_FMF.06.098.1 | FMF.06.098.1 | down | −1.97973165 | 1.41E−06 | P30291 |
| 52 | 52 | 0 | WEE1 | P30291-1 | wp117_DB0614 | DB0614 | down | −1.77707405 | 8.06E−09 | P30291 |
| 52 | 52 | 0 | WEE1 | P30291-1 | wp117_RSS0680 | RSS0680 | down | −1.84998741 | 7.15E−08 | P30291 |
| 52 | 52 | 0 | WEE1 | P30291-1 | wp118_ZNL.02.096 | ZNL.02.096 | down | −2.75038171 | 1.17E−07 | P30291 |
| 52 | 52 | 0 | WEE1 | P30291-1 | wp131_FMF.06.098.1 | FMF.06.098.1 | down | −2.1834263 | 4.68E−08 | P30291 |
| 52 | 52 | 0 | WEE1 | P30291-1 | wp131_SB1.G.192.1 | SB1.G.192.1 | down | −1.64733342 | 1.05E−06 | P30291 |
| 52 | 52 | 0 | WEE1 | P30291-1 | wp131_TL12.186 | TL12.188 | down | −2.57366808 | 8.66E−10 | P30291 |
| 52 | 52 | 0 | WEE1 | P30291-1 | wp133_SK.3.91_1 hr | SK.3.91 | down | −0.48657724 | 1.17E−06 | P30291 |
| 52 | 52 | 0 | WEE1 | P30291-1 | wp133_SK.3.91_2 hr | SK.3.91 | down | −1.21024551 | 6.78E−09 | P30291 |
| 52 | 52 | 0 | WEE1 | P30291-1 | wp133_SK.3.91_4 hr | SK.3.91 | down | −2.31420074 | 7.14E−11 | P30291 |
| 52 | 52 | 0 | WEE1 | P30291-1 | wp134_BSJ.04.178 | BSJ.04.178 | down | −0.56875056 | 7.04E−05 | P30291 |
| 52 | 52 | 0 | WEE1 | P30291-1 | wp137_BSJ.03.204 | BSJ.03.204 | down | −0.67590676 | 8.37E−06 | P30291 |
| 52 | 52 | 0 | WEE1 | P30291-1 | wp137_TL12.186 | TL12.186 | down | −2.51123565 | 5.72E−09 | P30291 |
| 52 | 52 | 0 | WEE1 | P30291-1 | wp137_TL13.97 | TL13.97 | down | −1.58516607 | 7.45E−08 | P30291 |
| 52 | 52 | 0 | WEE1 | P30291-1 | wp146_TMX.02.174 | TMX.02.174 | down | −1.14167731 | 0.00535894 | P30291 |
| 52 | 52 | 0 | WEE1 | P30291-1 | wp160_DB0646_8 hr | DB0646 | down | −0.35203028 | 0.00011077 | P30291 |
| 52 | 52 | 0 | WEE1 | P30291-1 | wp160_SK.3.91_1 hr | SK.3.91 | down | −0.55026437 | 1.84E−05 | P30291 |
| 52 | 52 | 0 | WEE1 | P30291-1 | wp160_SK.3.91_2 hr | SK.3.91 | down | −1.20112965 | 7.81E−07 | P30291 |
| 52 | 52 | 0 | WEE1 | P30291-1 | wp160_SK.3.91_4 hr | SK.3.91 | down | −1.99367271 | 1.00E−07 | P30291 |
| 52 | 52 | 0 | WEE1 | P30291-1 | wp172_SK.3.91 | SK.3.91 | down | −0.68531798 | 0.00092834 | P30291 |
| 52 | 52 | 0 | WEE1 | P30291-1 | wp196_BS.J.05.026 | BSJ.05.026 | down | −0.88756305 | 1.36E−07 | P30291 |
| 52 | 52 | 0 | WEE1 | P30291-1 | wp195_JWG.123 | JWG.123 | down | −1.36847374 | 1.21E−08 | P30291 |
| 52 | 52 | 0 | FER | P16591-3 | wp114_DB0646 | DB0646 | down | −0.5921274 | 0.00021958 | P16591 |
| 52 | 52 | 0 | FER | P16591-3 | wp114_FMF.06.098.1 | FMF.06.098.1 | down | −0.39857712 | 0.00104677 | P16591 |
| 52 | 52 | 0 | FER | P16591-3 | wp114_SK.3.91 | SK.3.91 | down | −2.30770871 | 8.92E−07 | P16591 |
| 52 | 52 | 0 | FER | P16591-3 | wp172_DB0646 | DB0646 | down | −1.0143376 | 5.34E−05 | P16591 |
| 52 | 52 | 0 | FER | P16591-3 | wp172_SB1.G.187 | SB1.G.187 | down | −1.39363178 | 1.09E−05 | P16591 |
| 52 | 52 | 0 | FER | P16591-3 | wp172_SK.3.91 | SK.3.91 | down | −2.81602332 | 1.09E−06 | P16591 |
| 52 | 52 | 0 | FER | P16591-1 | wp011_TL13.12 | TL13.12 | down | −0.84585457 | 1.07E−06 | P16591 |
| 52 | 52 | 0 | FER | P16591-1 | wp012_TI13.112 | TI13.112 | down | −0.52474886 | 0.00038904 | P16591 |
| 52 | 52 | 0 | FER | P16591-1 | wp016_LT2.49 | LT2.49 | down | −2.46660152 | 0.00013203 | P16591 |
| 52 | 52 | 0 | FER | P16591-1 | wp016_SK.3.87 | SK.3.87 | down | −1.96988287 | 0.00034287 | P16591 |
| 52 | 52 | 0 | FER | P16591-1 | wp016_SK.3.89 | SK.3.89 | down | −2.26199863 | 0.00019093 | P16591 |
| 52 | 52 | 0 | FER | P16591-1 | wp016_SK.3.91 | SK.3.91 | down | −2.64645518 | 9.79E−05 | P16591 |
| 52 | 52 | 0 | FER | P16591-1 | wp016_SK.3.91_1 uM | SK.3.91 | down | −3.3207279 | 3.68E−05 | P16591 |
| 52 | 52 | 0 | FER | P16591-1 | wp016_SK.3.93 | SK.3.93 | down | −2.64039598 | 9.87E−05 | P16591 |
| 52 | 52 | 0 | FER | P16591-1 | wp016_TL12.186 | TL12.186 | down | −2.72720626 | 8.59E−05 | P16591 |
| 52 | 52 | 0 | FER | P16591-1 | wp045_DB0614 | DB0614 | down | −0.76410656 | 0.00010901 | P16591 |
| 52 | 52 | 0 | FER | P16591-1 | wp045_DB0646 | DB0646 | down | −1.71162281 | 3.24E−06 | P16591 |
| 52 | 52 | 0 | FER | P16591-1 | wp045_DB0663 | DB0663 | down | −0.95805405 | 4.09E−05 | P16591 |
| 52 | 52 | 0 | FER | P16591-1 | wp056_SB1.G.187 | SB1.G.187 | down | −1.26663417 | 9.91E−05 | P16591 |
| 52 | 52 | 0 | FER | P16591-1 | wp081_TL13.150 | TL13.150 | down | −1.51104282 | 1.70E−06 | P16591 |
| 52 | 52 | 0 | FER | P16591-1 | wp103_DB0646 | DB0646 | down | −1.62764307 | 4.25E−06 | P16591 |
| 52 | 52 | 0 | FER | P16591-1 | wp103_SB1.G.187 | SB1.G.187 | down | −1.67177576 | 3.76E−06 | P16591 |
| 52 | 52 | 0 | FER | P16591-1 | wp103_SK.3.91 | SK.3.91 | down | −2.80991642 | 3.47E−07 | P16591 |
| 52 | 52 | 0 | FER | P16591-1 | wp105_DB0646 | DB0646 | down | −0.873745 | 3.15E−06 | P16591 |
| 52 | 52 | 0 | FER | P16591-1 | wp105_SB1.G.187 | SB1.G.187 | down | −1.43178909 | 3.03E−06 | P16591 |
| 52 | 52 | 0 | FER | P16591-1 | wp105_SK.3.91 | SK.3.91 | down | −2.85660857 | 2.73E−09 | P16591 |
| 52 | 52 | 0 | FER | P16591-1 | wp107_DB0646 | DB0646 | down | −1.72179799 | 3.38E−08 | P16591 |
| 52 | 52 | 0 | FER | P16591-1 | wp107_SB1.G.187 | SB1.G.187 | down | −1.75994663 | 6.59E−07 | P16591 |
| 52 | 52 | 0 | FER | P16591-1 | wp107_SK.3.91 | SK.3.91 | down | −2.65665989 | 4.04E−09 | P16591 |
| 52 | 52 | 0 | FER | P16591-1 | wp108_FMF.06.098.1 | FMF.06.098.1 | down | −0.35770306 | 0.00073025 | P16591 |
| 52 | 52 | 0 | FER | P16591-1 | wp117_DB0614 | DB0614 | down | −0.77248788 | 5.94E−06 | P16591 |
| 52 | 52 | 0 | FER | P16591-1 | wp117_DB0646 | DB0646 | down | −1.75314702 | 4.52E−07 | P16591 |
| 52 | 52 | 0 | FER | P16591-1 | wp131_FMF.06.098.1 | FMF.06.098.1 | down | −0.4870664 | 1.29E−06 | P16591 |
| 52 | 52 | 0 | FER | P16591-1 | wp131_TL12.186 | TL12.186 | down | −2.58273716 | 5.00E−08 | P16591 |
| 52 | 52 | 0 | FER | P16591-1 | wp133_SK.3.91_1 hr | SK.3.91 | down | −1.13213178 | 1.84E−06 | P16591 |
| 52 | 52 | 0 | FER | P16591-1 | wp133_SK.3.91_2 hr | SK.3.91 | down | −2.15322047 | 6.66E−07 | P16591 |
| 52 | 52 | 0 | FER | P16591-1 | wp133_SK.3.91_4 hr | SK.3.91 | down | −2.42623646 | 1.04E−08 | P16591 |
| 52 | 52 | 0 | FER | P16591-1 | wp137_TL12.186 | TL12.186 | down | −2.32926378 | 2.33E−07 | P16591 |
| 52 | 52 | 0 | FER | P16591-1 | wp137_TL13.97 | TL13.97 | down | −2.4116263 | 1.92E−07 | P16591 |
| 52 | 52 | 0 | FER | P16591-1 | wp146_TMX.02.138 | TMX.02.138 | down | −0.80833484 | 0.00089961 | P16591 |
| 52 | 52 | 0 | FER | P16591-1 | wp146_TMX.02.174 | TMX.02.174 | down | −1.08912281 | 0.000309 | P16591 |
| 52 | 52 | 0 | FER | P16591-1 | wp160_DB0646_2 hr | DB0646 | down | −0.53840235 | 0.00144764 | P16591 |
| 52 | 52 | 0 | FER | P16591-1 | wp160_DB0646_4 hr | DB0646 | down | −1.45441164 | 2.82E−05 | P16591 |
| 52 | 52 | 0 | FER | P16591-1 | wp160_DB0646_8 hr | DB0646 | down | −1.73731316 | 1.38E−05 | P16591 |
| 52 | 52 | 0 | FER | P16591-1 | wp160_SK.3.91_1 hr | SK.3.91 | down | −0.85338915 | 0.00023856 | P16591 |
| 52 | 52 | 0 | FER | P16591-1 | wp160_SK.3.91_2 hr | SK.3.91 | down | −1.87951611 | 1.00E−05 | P16591 |
| 52 | 52 | 0 | FER | P16591-1 | wp160_SK.3.91_4 hr | SK.3.91 | down | −1.98731768 | 8.00E−06 | P16591 |
| 52 | 52 | 0 | FER | P16591-1 | wp168_DB0661 | DB0661 | down | −0.61428568 | 2.58E−05 | P16591 |
| 52 | 52 | 0 | FER | P16591-1 | wp168_DB0662 | DB0662 | down | −1.48066029 | 1.07E−07 | P16591 |
| 52 | 52 | 0 | FER | P16591-1 | wp168_DB1113 | DB1113 | down | −1.11849305 | 2.55E−07 | P16591 |
| 52 | 52 | 0 | FER | P16591 | wp152_DB0646 | DB0646 | down | −1.69845147 | 6.90E−08 | P16591 |
| 52 | 52 | 0 | FER | P16591 | wp152_DB1114 | DB1114 | down | −1.1651995 | 5.51E−07 | P16591 |
| 47 | 47 | 0 | LIMK2 | P53671-3 | wp047_DD.03.106 | DD.03.106 | down | −0.96197778 | 3.11E−05 | P53671 |
| 47 | 47 | 0 | LIMK2 | P53671-3 | wp105_DB0646 | DB0646 | down | −0.72988983 | 6.59E−06 | P53671 |
| 47 | 47 | 0 | LIMK2 | P53671-3 | wp105_SK.3.91 | SK.3.91 | down | −0.84009752 | 8.42E−06 | P53671 |
| 47 | 47 | 0 | LIMK2 | P53671-3 | wp147_DD.03.106_2 uM | DD.03.106 | down | −1.08150311 | 1.01E−06 | P53671 |
| 47 | 47 | 0 | LIMK2 | P53671-3 | wp147_DD.03.108_1 uM | DD.03.106 | down | −0.87364567 | 3.03E−06 | P53671 |
| 47 | 47 | 0 | LIMK2 | P53671-3 | wp147_DD.03.106_4 uM | DD.03.106 | down | −0.93192947 | 2.18E−06 | P53671 |
| 47 | 47 | 0 | LIMK2 | P53671 | wp016_LT2.49 | LT2.49 | down | −0.95727476 | 8.41E−05 | P53671 |
| 47 | 47 | 0 | LIMK2 | P53671 | wp016_SK.3.87 | SK.3.87 | down | −0.37796863 | 0.00396575 | P53671 |
| 47 | 47 | 0 | LIMK2 | P53671 | wp016_SK.3.89 | SK.3.89 | down | −0.44401495 | 0.00209329 | P53671 |
| 47 | 47 | 0 | LIMK2 | P53671 | wp016_SK.3.91 | SK.3.91 | down | −1.06107867 | 5.40E−05 | P53671 |
| 47 | 47 | 0 | LIMK2 | P53671 | wp016_SK.3.91_1 uM | SK.3.91 | down | −1.44123196 | 1.44E−05 | P53671 |
| 47 | 47 | 0 | LIMK2 | P53671 | wp016_SK.3.93 | SK.3.93 | down | −1.00957361 | 6.69E−05 | P53671 |
| 47 | 47 | 0 | LIMK2 | P53671 | wp016_TL12.186 | TL12.186 | down | −0.73953998 | 0.00025265 | P53671 |
| 47 | 47 | 0 | LIMK2 | P53671 | wp045_DB0614 | DB0614 | down | −0.43698838 | 0.00094353 | P53671 |
| 47 | 47 | 0 | LIMK2 | P53671 | wp045_DB0646 | DB0646 | down | −1.64794175 | 3.07E−06 | P53671 |
| 47 | 47 | 0 | LIMK2 | P53671 | wp045_DB0663 | DB0663 | down | −1.36658798 | 6.97E−06 | P53671 |
| 47 | 47 | 0 | LIMK2 | P53671 | wp045_RSS0628 | RSS0628 | down | −0.71823813 | 0.00011451 | P53671 |
| 47 | 47 | 0 | LIMK2 | P53671 | wp056_DD.03.156 | DD.03.156 | down | −1.70286695 | 9.72E−06 | P53671 |
| 47 | 47 | 0 | LIMK2 | P53671 | wp074_dRAF.3 | dRAF.3 | down | −0.46564695 | 0.00340008 | P53671 |
| 47 | 47 | 0 | LIMK2 | P53671 | wp080_RSS0680 | RSS0680 | down | −0.88089478 | 0.00425583 | P53671 |
| 47 | 47 | 0 | LIMK2 | P53671 | wp081_SB1.G.192.1 | SB1.G.192.1 | down | −0.37101766 | 0.00011483 | P53671 |
| 47 | 47 | 0 | LIMK2 | P53671 | wp103_DB0646 | DB0646 | down | −1.56877525 | 4.16E−05 | P53671 |
| 47 | 47 | 0 | LIMK2 | P53671 | wp103_SK.3.91 | SK.3.91 | down | −1.43071381 | 6.31E−05 | P53671 |
| 47 | 47 | 0 | LIMK2 | P53671 | wp103_WH.10417.099 | WH.10417.099 | down | −0.5746865 | 0.00335619 | P53671 |
| 47 | 47 | 0 | LIMK2 | P53671 | wp107_DB0646 | DB0646 | down | −1.73837749 | 2.18E−06 | P53671 |
| 47 | 47 | 0 | LIMK2 | P53671 | wp107_SK.3.91 | SK.3.91 | down | −1.87814574 | 0.00024912 | P53671 |
| 47 | 47 | 0 | LIMK2 | P53671 | wp107_WH.10417.099 | WH.10417.099 | down | −0.37443083 | 0.00583785 | P53671 |
| 47 | 47 | 0 | LIMK2 | P53671 | wp108_FMF.06.098.1 | FMF.06.098.1 | down | −0.82259334 | 6.19E−06 | P53671 |
| 47 | 47 | 0 | LIMK2 | P53671 | wp114_FMF.06.098.1 | FMF.06.098.1 | down | −1.62920803 | 0.00043848 | P53671 |
| 47 | 47 | 0 | LIMK2 | P53671 | wp114_SK_3.91 | SK.3.91 | down | −0.84136465 | 0.00551637 | P53671 |
| 47 | 47 | 0 | LIMK2 | P53671 | wp117_DB0646 | DB0646 | down | −1.56470883 | 8.77E−06 | P53671 |
| 47 | 47 | 0 | LIMK2 | P53671 | wp117_RSS0680 | RSS0680 | down | −1.10901596 | 2.85E−05 | P53671 |
| 47 | 47 | 0 | LIMK2 | P53671 | wp131_FMF.06.098.1 | FMF.06.098.1 | down | −1.07691375 | 3.59E−08 | P53671 |
| 47 | 47 | 0 | LIMK2 | P53671 | wp131_SB1.G.192.1 | SB1.G.192.1 | down | −0.5543586 | 0.00104433 | P53671 |
| 47 | 47 | 0 | LIMK2 | P53671 | wp131_TL12.186 | TL12.186 | down | −1.32256158 | 2.84E−07 | P53671 |
| 47 | 47 | 0 | LIMK2 | P53671 | wp133_SK.3.91_2 hr | SK.3.91 | down | −0.75751742 | 7.96E−08 | P53671 |
| 47 | 47 | 0 | LIMK2 | P53671 | wp133_SK.3.91_4 hr | SK.3.91 | down | −1.34189829 | 2.47E−09 | P53671 |
| 47 | 47 | 0 | LIMK2 | P53671 | wp134_BSJ.05.037 | BSJ.05.037 | down | −0.49805496 | 0.00888482 | P53671 |
| 47 | 47 | 0 | LIMK2 | P53671 | wp137_DD.03.106 | DD.03.106 | down | −1.02604828 | 7.79E−07 | P53671 |
| 47 | 47 | 0 | LIMK2 | P53671 | wp137_TL12.186 | TL12.186 | down | −1.17915736 | 3.60E−07 | P53671 |
| 47 | 47 | 0 | LIMK2 | P53671 | wp137_TL13.97 | TL13.97 | down | −0.90538818 | 1.56E−06 | P53671 |
| 47 | 47 | 0 | LIMK2 | P53671 | wp149_ZNL.03.127 | ZNL.03.127 | down | −0.39444155 | 0.00015349 | P53671 |
| 47 | 47 | 0 | LIMK2 | P53671 | wp152_DB0646 | DB0646 | down | −1.56498983 | 1.97E−08 | P53671 |
| 47 | 47 | 0 | LIMK2 | P53671 | wp152_DB1114 | DB1114 | down | −1.62124176 | 1.62E−08 | P53671 |
| 47 | 47 | 0 | LIMK2 | P53671 | wp152_DB118430 | DB118430 | down | −0.45706277 | 1.69E−05 | P53671 |
| 47 | 47 | 0 | LIMK2 | P53671 | wp172_DB0646 | DB0646 | down | −1.09476419 | 0.00063324 | P53671 |
| 47 | 47 | 0 | LIMK2 | P53671 | wp172_SK.3.91 | SK.3.91 | down | −0.8984256 | 6.84E−05 | P53671 |
| 46 | 46 | 0 | BLK | P51451 | wp016_LT2.49 | LT2.49 | down | −1.61260212 | 3.63E−05 | P51451 |
| 46 | 46 | 0 | BLK | P51451 | wp016_SK.3.87 | SK.3.87 | down | −0.93285784 | 0.00037535 | P51451 |
| 46 | 46 | 0 | BLK | P51451 | wp016_SK.3.89 | SK.3.89 | down | −1.33525197 | 8.17E−05 | P51451 |
| 46 | 46 | 0 | BLK | P51451 | wp016_SK.3.91 | SK.3.91 | down | −1.44557581 | 5.81E−05 | P51451 |
| 46 | 46 | 0 | BLK | P51451 | wp016_SK.3.91_1 uM | SK.3.91 | down | −1.97711724 | 1.50E−05 | P51451 |
| 46 | 46 | 0 | BLK | P51451 | wp016_SK.3.93 | SK.3.93 | down | −1.50223675 | 4.92E−05 | P51451 |
| 46 | 46 | 0 | BLK | P51451 | wp016_TL12.186 | TL12.186 | down | −1.47538764 | 5.32E−05 | P51451 |
| 46 | 46 | 0 | BLK | P51451 | wp045_DB.3.291 | DB.3.291 | down | −0.5857418 | 0.00029325 | P51451 |
| 46 | 46 | 0 | BLK | P51451 | wp045_DB0646 | DB0646 | down | −1.31398467 | 8.86E−06 | P51451 |
| 46 | 46 | 0 | BLK | P51451 | wp045_DB0663 | DB0663 | down | −1.05754536 | 2.29E−05 | P51451 |
| 46 | 46 | 0 | BLK | P51451 | wp047_DD.03.119 | DD.03.119 | down | −1.85891832 | 2.41E−06 | P51451 |
| 46 | 46 | 0 | BLK | P51451 | wp056_SB1.G.187 | SB1.G.187 | down | −2.02068612 | 5.75E−06 | P51451 |
| 46 | 46 | 0 | BLK | P51451 | wp071_FMF.05.178.1 | FMF.05.178.1 | down | −0.34791528 | 0.00534091 | P51451 |
| 46 | 46 | 0 | BLK | P51451 | wp071_SB1.G.181 | SB1.G.181 | down | −2.33696927 | 6.85E−07 | P51451 |
| 46 | 46 | 0 | BLK | P51451 | wp071_SB1.G.192.1 | SB1.G.192.1 | down | −2.39858216 | 6.03E−07 | P51451 |
| 46 | 46 | 0 | BLK | P51451 | wp071_SB1.G.200 | SB1.G.200 | down | −1.52490782 | 5.52E−06 | P51451 |
| 46 | 46 | 0 | BLK | P51451 | wp074_dRAF.3 | dRAF.3 | down | −0.82164097 | 0.00107988 | P51451 |
| 46 | 46 | 0 | BLK | P51451 | wp081_SB1.G.192.1 | SB1.G.192.1 | down | −2.01281584 | 1.59E−07 | P51451 |
| 46 | 46 | 0 | BLK | P51451 | wp081_TL13.150 | TL13.150 | down | −1.43466248 | 1.02E−06 | P51451 |
| 46 | 46 | 0 | BLK | P51451 | wp103_DB0646 | DB0646 | down | −1.18987742 | 1.80E−05 | P51451 |
| 46 | 46 | 0 | BLK | P51451 | wp103_SB1.G.187 | SB1.G.187 | down | −2.09784985 | 1.34E−06 | P51451 |
| 46 | 46 | 0 | BLK | P51451 | wp103_SK.3.91 | SK.3.91 | down | −1.62481091 | 4.34E−06 | P51451 |
| 46 | 46 | 0 | BLK | P51451 | wp107_DB0646 | DB0646 | down | −1.25855028 | 1.82E−06 | P51451 |
| 46 | 46 | 0 | BLK | P51451 | wp107_SB1.G.187 | SB1.G.187 | down | −1.91613097 | 1.67E−07 | P51451 |
| 46 | 46 | 0 | BLK | P51451 | wp107_SK.3.91 | SK.3.91 | down | −1.64871436 | 2.01E−07 | P51451 |
| 46 | 46 | 0 | BLK | P51451 | wp117_DB0646 | DB0646 | down | −1.27493217 | 1.59E−06 | P51451 |
| 46 | 46 | 0 | BLK | P51451 | wp131_SB1.G.192.1 | SB1.G.192.1 | down | −2.46536587 | 4.77E−08 | P51451 |
| 46 | 46 | 0 | BLK | P51451 | wp131_TL12.186 | TL12.186 | down | −2.49717109 | 2.60E−07 | P51451 |
| 46 | 46 | 0 | BLK | P51451 | wp133_SK.3.91_1 hr | SK.3.91 | down | −0.37267755 | 5.36E−06 | P51451 |
| 46 | 46 | 0 | BLK | P51451 | wp133_SK.3.91_2 hr | SK.3.91 | down | −0.82170436 | 9.73E−08 | P51451 |
| 46 | 46 | 0 | BLK | P51451 | wp133_SK.3.91_4 hr | SK.3.91 | down | −1.48960808 | 1.18E−09 | P51451 |
| 46 | 46 | 0 | BLK | P51451 | wp134_DB118430 | DB118430 | down | −0.58261334 | 6.05E−06 | P51451 |
| 46 | 46 | 0 | BLK | P51451 | wp137_TL12.186 | TL12.186 | down | −2.1154457 | 1.09E−08 | P51451 |
| 46 | 46 | 0 | BLK | P51451 | wp137_TL13.97 | TL13.97 | down | −2.21874209 | 8.37E−09 | P51451 |
| 46 | 46 | 0 | BLK | P51451 | wp149_ZNL.03.127 | ZNL.03.127 | down | −0.50244855 | 0.00014088 | P51451 |
| 46 | 46 | 0 | BLK | P51451 | wp152_DB0646 | DB0646 | down | −1.51144006 | 2.58E−07 | P51451 |
| 46 | 46 | 0 | BLK | P51451 | wp152_DB1114 | DB1114 | down | −1.31295037 | 5.60E−07 | P51451 |
| 46 | 46 | 0 | BLK | P51451 | wp152_DB118430 | DB118430 | down | −0.90230889 | 4.38E−06 | P51451 |
| 46 | 46 | 0 | BLK | P51451 | wp160_DB0646_2 hr | DB0646 | down | −0.53153367 | 0.00010069 | P51451 |
| 46 | 46 | 0 | BLK | P51451 | wp160_DB0646_4 hr | DB0646 | down | −1.09238244 | 5.53E−06 | P51451 |
| 46 | 46 | 0 | BLK | P51451 | wp160_DB0646_8 hr | DB0646 | down | −1.43411695 | 1.84E−06 | P51451 |
| 46 | 46 | 0 | BLK | P51451 | wp160_SK.3.91_2 hr | SK.3.91 | down | −0.87437146 | 1.36E−05 | P51451 |
| 46 | 46 | 0 | BLK | P51451 | wp160_SK.3.91_4 hr | SK.3.91 | down | −1.67778123 | 9.73E−07 | P51451 |
| 46 | 46 | 0 | BLK | P51451 | wp168_DB0661 | DB0661 | down | −1.42805981 | 7.33E−08 | P51451 |
| 46 | 46 | 0 | BLK | P51451 | wp168_DB0662 | DB0662 | down | −1.65156973 | 2.36E−08 | P51451 |
| 46 | 46 | 0 | BLK | P51451 | wp168_DB1113 | DB1113 | down | −1.32007222 | 1.69E−07 | P51451 |
| 42 | 42 | 0 | CDK6 | Q00534 | wp005_BSJ.01.147 | BSJ.01.147 | down | −2.82030879 | 1.59E−07 | Q00534 |
| 42 | 42 | 0 | CDK6 | Q00534 | wp005_YKL.6.102 | YKL.6.102 | down | −2.45239432 | 1.04E−09 | Q00534 |
| 42 | 42 | 0 | CDK6 | Q00534 | wp016_LT2.49 | LT2.49 | down | −0.65310486 | 1.31E−05 | Q00534 |
| 42 | 42 | 0 | CDK6 | Q00534 | wp016_SK.3.89 | SK.3.89 | down | −0.33964948 | 0.0002168 | Q00534 |
| 42 | 42 | 0 | CDK6 | Q00534 | wp016_SK.3.91 | SK.3.91 | down | −0.88321573 | 3.52E−06 | Q00534 |
| 42 | 42 | 0 | CDK6 | Q00534 | wp016_SK.3.91_1 uM | SK.3.91 | down | −1.1491542 | 1.12E−06 | Q00534 |
| 42 | 42 | 0 | CDK6 | Q00534 | wp016_SK.3.93 | SK.3.93 | down | −0.85848895 | 3.98E−06 | Q00534 |
| 42 | 42 | 0 | CDK6 | Q00534 | wp016_TL12.186 | TL12.186 | down | −0.58312155 | 2.13E−05 | Q00534 |
| 42 | 42 | 0 | CDK6 | Q00534 | wp045_DB0614 | DB0614 | down | −2.26508782 | 6.82E−09 | Q00534 |
| 42 | 42 | 0 | CDK6 | Q00534 | wp045_RSS0628 | RSS0628 | down | −1.7427234 | 2.16E−08 | Q00534 |
| 42 | 42 | 0 | CDK6 | Q00534 | wp047_DD.03.106 | DD.03.106 | down | −1.08641386 | 1.11E−07 | Q00534 |
| 42 | 42 | 0 | CDK6 | Q00534 | wp080_RSS0680 | RSS0680 | down | −2.16991963 | 8.34E−09 | Q00534 |
| 42 | 42 | 0 | CDK6 | Q00534 | wp084_BSJ.01.147 | BSJ.01.147 | down | −3.16351088 | 1.53E−08 | Q00534 |
| 42 | 42 | 0 | CDK6 | Q00534 | wp084_BSJ.03.123 | BSJ.03.123 | down | −3.05777151 | 3.62E−10 | Q00534 |
| 42 | 42 | 0 | CDK6 | Q00534 | wp084_BSJ.03.204 | BSJ.03.204 | down | −3.57383964 | 3.05E−10 | Q00534 |
| 42 | 42 | 0 | CDK6 | Q00534 | wp084_BSJ.04.132 | BSJ.04.132 | down | −0.75314666 | 2.58E−06 | Q00534 |
| 42 | 42 | 0 | CDK6 | Q00534 | wp088_SB1.G.194 | SB1.G.194 | down | −0.47309967 | 0.00031615 | Q00534 |
| 42 | 42 | 0 | CDK6 | Q00534 | wp103_SB1.G.187 | SB1.G.187 | down | −0.34597256 | 0.00071054 | Q00534 |
| 42 | 42 | 0 | CDK6 | Q00534 | wp103_SK.3.91 | SK.3.91 | down | −0.93490244 | 8.21E−06 | Q00534 |
| 42 | 42 | 0 | CDK6 | Q00534 | wp104_TMX.01.160.1_0.25 | TMX.01.160.1 | down | −0.86597322 | 6.49E−07 | Q00534 |
| 42 | 42 | 0 | CDK6 | Q00534 | wp104_TMX.01.160.1_1 | TMX.01.160.1 | down | −0.84250525 | 7.47E−07 | Q00534 |
| 42 | 42 | 0 | CDK6 | Q00534 | wp105_SK.3.91 | SK.3.91 | down | −0.54214131 | 0.00011271 | Q00534 |
| 42 | 42 | 0 | CDK6 | Q00534 | wp107_SK.3.91 | SK.3.91 | down | −1.01197262 | 3.16E−07 | Q00534 |
| 42 | 42 | 0 | CDK6 | Q00534 | wp108_FMF.06.098.1 | FMF.06.098.1 | down | −0.35881977 | 0.00017123 | Q00534 |
| 42 | 42 | 0 | CDK6 | Q00534 | wp117_DB0614 | DB0614 | down | −2.0792144 | 9.47E−09 | Q00534 |
| 42 | 42 | 0 | CDK6 | Q00534 | wp117_RSS0680 | RSS0680 | down | −2.16865165 | 1.56E−09 | Q00534 |
| 42 | 42 | 0 | CDK6 | Q00534 | wp131_FMF.06.098.1 | FMF.06.098.1 | down | −0.53773599 | 2.00E−05 | Q00534 |
| 42 | 42 | 0 | CDK6 | Q00534 | wp131_TL12.186 | TL12.186 | down | −0.98923743 | 4.97E−07 | Q00534 |
| 42 | 42 | 0 | CDK6 | Q00534 | wp133_SK.3.91_1 hr | SK.3.91 | down | −0.35424297 | 4.51E−07 | Q00534 |
| 42 | 42 | 0 | CDK6 | Q00534 | wp133_SK.3.91_2 hr | SK.3.91 | down | −0.64036034 | 4.14E−08 | Q00534 |
| 42 | 42 | 0 | CDK6 | Q00534 | wp133_SK.3.91_4 hr | SK.3.91 | down | −1.05021923 | 2.42E−10 | Q00534 |
| 42 | 42 | 0 | CDK6 | Q00534 | wp137_BSJ.03.204 | BSJ.03.204 | down | −2.86563887 | 2.07E−11 | Q00534 |
| 42 | 42 | 0 | CDK6 | Q00534 | wp137_DD.03.106 | DD.03.106 | down | −1.08319852 | 4.75E−09 | Q00534 |
| 42 | 42 | 0 | CDK6 | Q00534 | wp137_TL12.186 | TL12.186 | down | −0.87244887 | 1.59E−08 | Q00534 |
| 42 | 42 | 0 | CDK6 | Q00534 | wp137_TL13.97 | TL13.97 | down | −0.59671265 | 1.32E−07 | Q00534 |
| 42 | 42 | 0 | CDK6 | Q00534 | wp147_DD.03.108_2 uM | DD.03.106 | down | −1.34760783 | 7.88E−08 | Q00534 |
| 42 | 42 | 0 | CDK6 | Q00534 | wp147_DD.03.106_1 uM | DD.03.106 | down | −0.8934037 | 6.54E−07 | Q00534 |
| 42 | 42 | 0 | CDK6 | Q00534 | wp147_DD.03.106_4 uM | DD.03.106 | down | −1.15870576 | 1.72E−07 | Q00534 |
| 42 | 42 | 0 | CDK6 | Q00534 | wp160_SK.3.91_1 hr | SK.3.91 | down | −0.32547569 | 6.58E−05 | Q00534 |
| 42 | 42 | 0 | CDK6 | Q00534 | wp160_SK.3.91_2 hr | SK.3.91 | down | −0.59478608 | 5.79E−06 | Q00534 |
| 42 | 42 | 0 | CDK6 | Q00534 | wp160_SK.3.91_4 hr | SK.3.91 | down | −1.13254937 | 4.26E−07 | Q00534 |
| 42 | 42 | 0 | CDK6 | Q00534 | wp164_SB1.G.194 | SB1.G.194 | down | −1.22969958 | 6.14E−07 | Q00534 |
| 41 | 40 | 1 | LIMK1 | P53667-1 | wp045_DB0646 | DB0646 | down | −0.52928399 | 0.00044638 | P53667 |
| 41 | 40 | 1 | LIMK1 | P53667-1 | wp047_DD.03.106 | DD.03.106 | down | −1.63729595 | 0.0011134 | P53667 |
| 41 | 40 | 1 | LIMK1 | P53667-1 | wp056_DD.03.107 | DD.03.107 | down | −0.95944118 | 0.00131444 | P53667 |
| 41 | 40 | 1 | LIMK1 | P53667-1 | wp056_DD.03.156 | DD.03.156 | down | −1.03684401 | 0.00096667 | P53667 |
| 41 | 40 | 1 | LIMK1 | P53667-1 | wp071_SB1.G.181 | SB1.G.181 | down | −0.68603687 | 0.00099745 | P53667 |
| 41 | 40 | 1 | LIMK1 | P53667-1 | wp071_SB1.G.200 | SB1.G.200 | down | −0.7749743 | 0.00057135 | P53667 |
| 41 | 40 | 1 | LIMK1 | P53667-1 | wp081_TL13.178 | TL13.178 | down | −0.33594721 | 0.00998705 | P53667 |
| 41 | 40 | 1 | LIMK1 | P53667-1 | wp103_DB0646 | DB0646 | down | −1.49737502 | 1.40E−05 | P53667 |
| 41 | 40 | 1 | LIMK1 | P53667-1 | wp103_SK.3.91 | SK.3.91 | down | −1.23840045 | 3.32E−05 | P53667 |
| 41 | 40 | 1 | LIMK1 | P53667-1 | wp105_DB0646 | DB0646 | down | −0.56409103 | 0.00020909 | P53667 |
| 41 | 40 | 1 | LIMK1 | P53667-1 | wp105_SK.3.91 | SK.3.91 | down | −1.46377571 | 8.77E−07 | P53667 |
| 41 | 40 | 1 | LIMK1 | P53667-1 | wp107_DB0646 | DB0646 | down | −0.97681715 | 3.15E−05 | P53667 |
| 41 | 40 | 1 | LIMK1 | P53667-1 | wp107_SK.3.91 | SK.3.91 | down | −0.87042034 | 1.99E−05 | P53667 |
| 41 | 40 | 1 | LIMK1 | P53667-1 | wp114_SK.3.91 | SK.3.91 | down | −0.54603649 | 0.00267092 | P53667 |
| 41 | 40 | 1 | LIMK1 | P53667-1 | wp117_DB0646 | DB0646 | down | −1.41245855 | 5.51E−06 | P53667 |
| 41 | 40 | 1 | LIMK1 | P53667-1 | wp131_TL12.186 | TL12.186 | down | −1.05541712 | 1.82E−07 | P53667 |
| 41 | 40 | 1 | LIMK1 | P53667-1 | wp133_SK.3.91_1 hr | SK.3.91 | down | −0.41168975 | 0.00024024 | P53667 |
| 41 | 40 | 1 | LIMK1 | P53667-1 | wp133_SK.3.91_2 hr | SK.3.91 | down | −0.80263601 | 3.90E−06 | P53667 |
| 41 | 40 | 1 | LIMK1 | P53667-1 | wp133_SK.3.91_4 hr | SK.3.91 | down | −1.37069074 | 2.51E−07 | P53667 |
| 41 | 40 | 1 | LIMK1 | P53667-1 | wp137_DD.03.106 | DD.03.106 | down | −1.98060157 | 1.23E−06 | P53667 |
| 41 | 40 | 1 | LIMK1 | P53667-1 | wp137_DD.03.107 | DD.03.107 | down | −1.22209666 | 1.74E−05 | P53667 |
| 41 | 40 | 1 | LIMK1 | P53667-1 | wp137_TL12.186 | TL12.186 | down | −0.87335821 | 0.00010658 | P53667 |
| 41 | 40 | 1 | LIMK1 | P53667-1 | wp137_TL13.97 | TL13.97 | down | −1.17329608 | 2.18E−05 | P53667 |
| 41 | 40 | 1 | LIMK1 | P53667-1 | wp147_DD.03.106_2 uM | DD.03.106 | down | −2.19977631 | 7.22E−07 | P53667 |
| 41 | 40 | 1 | LIMK1 | P53667-1 | wp147_DD.03.106_1 uM | DD.03.106 | down | −2.95903508 | 1.57E−07 | P53667 |
| 41 | 40 | 1 | LIMK1 | P53667-1 | wp147_DD.03.108_4 uM | DD.03.106 | down | −0.63701715 | 0.00037517 | P53667 |
| 41 | 40 | 1 | LIMK1 | P53667-1 | wp147_DD.03.107_1 uM | DD.03.107 | down | −0.9691272 | 4.71E−05 | P53667 |
| 41 | 40 | 1 | LIMK1 | P53667-1 | wp147_DD.03.107_2 uM | DD.03.107 | down | −1.37545255 | 7.99E−06 | P53667 |
| 41 | 40 | 1 | LIMK1 | P53667-1 | wp147_DD.03.107_4 uM | DD.03.107 | down | −1.53646712 | 4.54E−06 | P53667 |
| 41 | 40 | 1 | LIMK1 | P53667-1 | wp149_ZNL.03.127 | ZNL.03.127 | down | −0.47637664 | 0.00138834 | P53667 |
| 41 | 40 | 1 | LIMK1 | P53667-1 | wp160_DB0846_2 hr | DB0646 | down | −0.73756896 | 0.00376091 | P53667 |
| 41 | 40 | 1 | LIMK1 | P53667-1 | wp160_DB0646_4 hr | DB0646 | down | −1.56290507 | 0.00020525 | P53667 |
| 41 | 40 | 1 | LIMK1 | P53667-1 | wp160_DB0646_8 hr | DB0646 | down | −2.35956128 | 3.95E−05 | P53667 |
| 41 | 40 | 1 | LIMK1 | P53667-1 | wp160_SK.3.91_2 hr | SK.3.91 | down | −0.8633992 | 0.00208085 | P53667 |
| 41 | 40 | 1 | LIMK1 | P53667-1 | wp160_SK.3.91_4 hr | SK.3.91 | down | −1.36577373 | 0.00035027 | P53667 |
| 41 | 40 | 1 | LIMK1 | P53667-1 | wp168_DB0661 | DB0661 | down | −1.11582341 | 2.39E−05 | P53667 |
| 41 | 40 | 1 | LIMK1 | P53667-1 | wp168_DB0662 | DB0662 | down | −1.91055853 | 3.04E−06 | P53667 |
| 41 | 40 | 1 | LIMK1 | P53667-1 | wp168_DB1113 | DB1113 | down | −1.14411663 | 2.09E−05 | P53667 |
| 41 | 40 | 1 | LIMK1 | P53667-1 | wp172_DB0646 | DB0646 | down | −1.31983784 | 1.63E−05 | P53667 |
| 41 | 40 | 1 | LIMK1 | P53667-1 | wp172_SK.3.91 | SK.3.91 | down | −1.86880069 | 1.39E−06 | P53667 |
| 41 | 41 | 0 | GAK | O14976 | wp016_SK.3.91 | SK.3.91 | down | −0.5011104 | 2.67E−05 | O14976 |
| 41 | 41 | 0 | GAK | O14976 | wp018_SK.3.91_1 uM | SK.3.91 | down | −0.35262316 | 0.00012093 | O14976 |
| 41 | 41 | 0 | GAK | O14976 | wp016_SK.3.93 | SK.3.93 | down | −0.43339593 | 4.99E−05 | O14976 |
| 41 | 41 | 0 | GAK | O14976 | wp016_TL12.186 | TL12.186 | down | −0.33538767 | 0.00014977 | O14976 |
| 41 | 41 | 0 | GAK | O14976 | wp045_DB0614 | DB0614 | down | −0.33053838 | 0.00051994 | O14976 |
| 41 | 41 | 0 | GAK | O14976 | wp045_DB0646 | DB0646 | down | −0.64236159 | 2.99E−05 | O14976 |
| 41 | 41 | 0 | GAK | O14976 | wp045_DB0663 | DB0663 | down | −0.6909978 | 2.18E−05 | O14976 |
| 41 | 41 | 0 | GAK | O14976 | wp056_SB1.G.187 | SB1.G.187 | down | −1.54275392 | 3.76E−06 | O14976 |
| 41 | 41 | 0 | GAK | O14976 | wp071_JWG.118 | JWG.118 | down | −1.97222022 | 7.66E−08 | O14976 |
| 41 | 41 | 0 | GAK | O14976 | wp071_SB1.G.192.1 | SB1.G.192.1 | down | −1.76546148 | 1.32E−07 | O14976 |
| 41 | 41 | 0 | GAK | O14976 | wp080_RSS0680 | RSS0680 | down | −0.7772934 | 1.10E−06 | O14976 |
| 41 | 41 | 0 | GAK | O14976 | wp081_SB1.G.192.1 | SB1.G.192.1 | down | −1.40863529 | 1.96E−08 | O14976 |
| 41 | 41 | 0 | GAK | O14976 | wp081_TL13.150 | TL13.150 | down | −1.01424781 | 1.20E−07 | O14976 |
| 41 | 41 | 0 | GAK | O14976 | wp088_JWG.122 | JWG.122 | down | −0.35856807 | 0.00014567 | O14976 |
| 41 | 41 | 0 | GAK | O14976 | wp103_DB0646 | DB0646 | down | −0.46843606 | 2.96E−05 | O14976 |
| 41 | 41 | 0 | GAK | O14976 | wp103_SB1.G.187 | SB1.G.187 | down | −1.47806268 | 1.54E−07 | O14976 |
| 41 | 41 | 0 | GAK | O14976 | wp105_DB0646 | DB0646 | down | −0.33671999 | 0.00023672 | O14976 |
| 41 | 41 | 0 | GAK | O14976 | wp105_SB1.G.187 | SB1.G.187 | down | −1.08822908 | 3.34E−06 | O14976 |
| 41 | 41 | 0 | GAK | O14976 | wp105_SK.3.91 | SK.3.91 | down | −0.38321027 | 9.55E−05 | O14976 |
| 41 | 41 | 0 | GAK | O14976 | wp107_DB0646 | DB0646 | down | −0.55635606 | 2.19E−07 | O14976 |
| 41 | 41 | 0 | GAK | O14976 | wp107_SB1.G.187 | SB1.G.187 | down | −1.76783158 | 7.55E−09 | O14976 |
| 41 | 41 | 0 | GAK | O14976 | wp108_FMF.06.098.1 | FMF.06.098.1 | down | −0.415636 | 0.00041245 | O14976 |
| 41 | 41 | 0 | GAK | O14976 | wp114_DB0646 | DB0646 | down | −0.33914951 | 0.0001611 | O14976 |
| 41 | 41 | 0 | GAK | O14976 | wp114_SB1.G.187 | SB1.G.187 | down | −0.35778772 | 0.00013002 | O14976 |
| 41 | 41 | 0 | GAK | O14976 | wp117_DB0614 | DB0614 | down | −0.41964743 | 0.00026265 | O14976 |
| 41 | 41 | 0 | GAK | O14978 | wp117_DB0646 | DB0646 | down | −0.5868103 | 5.00E−05 | O14976 |
| 41 | 41 | 0 | GAK | O14976 | wp117_RSS0680 | RSS0680 | down | −0.68527741 | 1.54E−05 | O14976 |
| 41 | 41 | 0 | GAK | O14976 | wp131_FMF.06.098.1 | FMF.06.098.1 | down | −0.34690626 | 1.20E−06 | O14976 |
| 41 | 41 | 0 | GAK | O14976 | wp131_SB1.G.192.1 | SB1.G.192.1 | down | −1.47469479 | 7.71E−10 | O14976 |
| 41 | 41 | 0 | GAK | O14976 | wp134_BSJ.04.178 | BSJ.04.178 | down | −2.40226375 | 1.80E−10 | O14976 |
| 41 | 41 | 0 | GAK | O14976 | wp152_DB0646 | DB0646 | down | −0.51174579 | 3.50E−06 | O14976 |
| 41 | 41 | 0 | GAK | O14976 | wp152_DB1114 | DB1114 | down | −0.99941295 | 8.82E−08 | O14976 |
| 41 | 41 | 0 | GAK | O14976 | wp160_DB0646_4 hr | DB0646 | down | −0.47137857 | 1.32E−05 | O14976 |
| 41 | 41 | 0 | GAK | O14976 | wp160_DB0646_8 hr | DB0646 | down | −0.81488553 | 1.44E−06 | O14976 |
| 41 | 41 | 0 | GAK | O14976 | wp160_SK.3.91_4 hr | SK.3.91 | down | −0.32412575 | 5.95E−05 | O14976 |
| 41 | 41 | 0 | GAK | O14976 | wp168_DB0662 | DB0662 | down | −0.43731219 | 4.61E−07 | O14976 |
| 41 | 41 | 0 | GAK | O14976 | wp168_DB1113 | DB1113 | down | −1.02885118 | 1.49E−08 | O14976 |
| 41 | 41 | 0 | GAK | O14976 | wp172_DB0646 | DB0646 | down | −0.3449661 | 0.00015899 | O14976 |
| 41 | 41 | 0 | GAK | O14976 | wp172_SB1.G.187 | SB1.G.187 | down | −1.006514 | 2.42E−07 | O14976 |
| 41 | 41 | 0 | GAK | O14976 | wp195_BS.J.05.026 | BSJ.05.026 | down | −0.68186445 | 8.20E−07 | O14976 |
| 41 | 41 | 0 | GAK | O14976 | wp195_JWG.123 | JWG.123 | down | −0.52455392 | 1.46E−06 | O14976 |
| 39 | 39 | 0 | AAK1 | Q2M218-1 | wp016_LT2.49 | LT2.49 | down | −1.7778276 | 1.56E−06 | Q2M218 |
| 39 | 39 | 0 | AAK1 | Q2M218-1 | wp016_SK.3.87 | SK.3.87 | down | −0.7306945 | 7.33E−05 | Q2M218 |
| 39 | 39 | 0 | AAK1 | Q2M218-1 | wp016_SK.3.89 | SK.3.89 | down | −1.21939421 | 8.02E−06 | Q2M218 |
| 39 | 39 | 0 | AAK1 | Q2M218-1 | wp016_SK.3.91 | SK.3.91 | down | −1.36352796 | 4.94E−06 | Q2M218 |
| 39 | 39 | 0 | AAK1 | Q2M218-1 | wp016_SK.3.91_1 uM | SK.3.91 | down | −1.11385015 | 1.19E−05 | Q2M218 |
| 39 | 39 | 0 | AAK1 | Q2M218-1 | wp016_SK.3.93 | SK.3.93 | down | −1.58828042 | 2.54E−06 | Q2M218 |
| 39 | 39 | 0 | AAK1 | Q2M218-1 | wp016_TL12.186 | TL12.186 | down | −1.33795454 | 5.36E−06 | Q2M218 |
| 39 | 39 | 0 | AAK1 | Q2M218-1 | wp045_DB0614 | DB0614 | down | −1.86280565 | 3.13E−07 | Q2M218 |
| 39 | 39 | 0 | AAK1 | Q2M218-1 | wp045_RSS0628 | RSS0628 | down | −1.24006713 | 1.87E−06 | Q2M218 |
| 39 | 39 | 0 | AAK1 | Q2M218-1 | wp056_SB1.G.190 | SB1.G.190 | down | −1.48911764 | 4.30E−06 | Q2M218 |
| 39 | 39 | 0 | AAK1 | Q2M218-1 | wp071_JWG.118 | JWG.118 | down | −3.04436071 | 8.95E−09 | Q2M218 |
| 39 | 39 | 0 | AAK1 | Q2M218-1 | wp080_RSS0680 | RSS0680 | down | −1.39211284 | 2.54E−07 | Q2M218 |
| 39 | 39 | 0 | AAK1 | Q2M218-1 | wp088_JWG.122 | JWG.122 | down | −0.63353214 | 3.87E−06 | Q2M218 |
| 39 | 39 | 0 | AAK1 | Q2M218-1 | wp088_SB1.G.194 | SB1.G.194 | down | −0.34820154 | 6.98E−05 | Q2M218 |
| 39 | 39 | 0 | AAK1 | Q2M218-1 | wp103_SK.3.91 | SK.3.91 | down | −0.77628395 | 5.50E−05 | Q2M218 |
| 39 | 39 | 0 | AAK1 | Q2M218-1 | wp105_SK.3.91 | SK.3.91 | down | −1.57454698 | 2.50E−09 | Q2M218 |
| 39 | 39 | 0 | AAK1 | Q2M218-1 | wp107_SK.3.91 | SK.3.91 | down | −1.05887558 | 7.28E−08 | Q2M218 |
| 39 | 39 | 0 | AAK1 | Q2M218-1 | wp108_FMF.06.098.1 | FMF.06.098.1 | down | −0.43459605 | 0.00043593 | Q2M218 |
| 39 | 39 | 0 | AAK1 | Q2M218-1 | wp114_FMF.06.098.1 | FMF.06.098.1 | down | −0.48497849 | 0.00094162 | Q2M218 |
| 39 | 39 | 0 | AAK1 | Q2M218-1 | wp114_SK.3.91 | SK.3.91 | down | −0.82207604 | 0.00011615 | Q2M218 |
| 39 | 39 | 0 | AAK1 | Q2M218-1 | wp117_DB0614 | DB0614 | down | −1.66214492 | 6.66E−08 | Q2M218 |
| 39 | 39 | 0 | AAK1 | Q2M218-1 | wp117_RSS0680 | RSS0680 | down | −1.05013249 | 1.24E−07 | Q2M218 |
| 39 | 39 | 0 | AAK1 | Q2M218-1 | wp131_FMF.06.098.1 | FMF.06.098.1 | down | −0.72933047 | 9.25E−07 | Q2M218 |
| 39 | 39 | 0 | AAK1 | Q2M218-1 | wp131_TL12.186 | TL12.186 | down | −1.51999609 | 3.80E−08 | Q2M218 |
| 39 | 39 | 0 | AAK1 | Q2M218-1 | wp133_SK.3.91_2 hr | SK.3.91 | down | −0.55056142 | 2.87E−08 | Q2M218 |
| 39 | 39 | 0 | AAK1 | Q2M218-1 | wp133_SK.3.91_4 hr | SK.3.91 | down | −1.10478909 | 1.44E−09 | Q2M218 |
| 39 | 39 | 0 | AAK1 | Q2M218-1 | wp134_BSJ.04.178 | BSJ.04.178 | down | −3.34172286 | 1.23E−09 | Q2M218 |
| 39 | 39 | 0 | AAK1 | Q2M218-1 | wp134_JWG.120 | JWG.120 | down | −0.33622525 | 0.00057891 | Q2M218 |
| 39 | 39 | 0 | AAK1 | Q2M218-1 | wp137_TL12.186 | TL12.186 | down | −1.30993896 | 5.26E−09 | Q2M218 |
| 39 | 39 | 0 | AAK1 | Q2M218-1 | wp137_TL13.97 | TL13.97 | down | −2.4785785 | 1.49E−10 | Q2M218 |
| 39 | 39 | 0 | AAK1 | Q2M218-1 | wp146_TMX.02.138 | TMX.02.138 | down | −1.12934095 | 9.17E−05 | Q2M218 |
| 39 | 39 | 0 | AAK1 | Q2M218-1 | wp146_TMX.02.174 | TMX.02.174 | down | −1.61978184 | 2.46E−05 | Q2M218 |
| 39 | 39 | 0 | AAK1 | Q2M218-1 | wp160_SK.3.91_2 hr | SK.3.91 | down | −0.51525749 | 3.77E−05 | Q2M218 |
| 39 | 39 | 0 | AAK1 | Q2M218-1 | wp160_SK.3.91_4 hr | SK.3.91 | down | −1.00777589 | 2.51E−06 | Q2M218 |
| 39 | 39 | 0 | AAK1 | Q2M218-1 | wp164_SB1.G.194 | SB1.G.194 | down | −0.3789904 | 1.76E−05 | Q2M218 |
| 39 | 39 | 0 | AAK1 | Q2M218-1 | wp172_SK.3.91 | SK.3.91 | down | −0.97545846 | 8.28E−08 | Q2M218 |
| 39 | 39 | 0 | AAK1 | Q2M218-1 | wp195_BJG.04.203 | BJG.04.203 | down | −1.3360133 | 1.06E−08 | Q2M218 |
| 39 | 39 | 0 | AAK1 | Q2M218-1 | wp195_BSJ.05.026 | BSJ.05.026 | down | −3.07321215 | 1.00E−10 | Q2M218 |
| 39 | 39 | 0 | AAK1 | Q2M218-1 | wp195_JWG.123 | JWG.123 | down | −1.09113715 | 3.29E−08 | Q2M218 |
| 39 | 39 | 0 | LCK | P06239-3 | wp016_LT2.49 | LT2.49 | down | −0.6185539 | 4.30E−05 | P06239 |
| 39 | 39 | 0 | LCK | P06239-3 | wp016_SK.3.87 | SK.3.87 | down | −0.34840792 | 0.00049556 | P06239 |
| 39 | 39 | 0 | LCK | P06239-3 | wp016_SK.3.89 | SK.3.89 | down | −0.44902313 | 0.00016966 | P06239 |
| 39 | 39 | 0 | LCK | P06239-3 | wp016_SK.3.91 | SK.3.91 | down | −0.91892015 | 7.76E−06 | P06239 |
| 39 | 39 | 0 | LCK | P06239-3 | wp016_SK.3.91_1 uM | SK.3.91 | down | −1.57909743 | 7.36E−07 | P06239 |
| 39 | 39 | 0 | LCK | P06239-3 | wp016_SK.3.93 | SK.3.93 | down | −0.77979861 | 1.58E−05 | P06239 |
| 39 | 39 | 0 | LCK | P06239-3 | wp016_TL12.186 | TL12.186 | down | −0.71293729 | 2.33E−05 | P06239 |
| 39 | 39 | 0 | LCK | P06239-3 | wp045_DB0614 | DB0614 | down | −0.33004179 | 0.00059176 | P06239 |
| 39 | 39 | 0 | LCK | P06239-3 | wp045_DB0646 | DB0646 | down | −0.56314091 | 6.01E−05 | P06239 |
| 39 | 39 | 0 | LCK | P06239-3 | wp045_DB0663 | DB0663 | down | −0.58233207 | 5.20E−05 | P06239 |
| 39 | 39 | 0 | LCK | P06239-3 | wp047_DD.03.119 | DD.03.119 | down | −1.78691057 | 1.73E−07 | P06239 |
| 39 | 39 | 0 | LCK | P06239-3 | wp056_SB1.G.187 | SB1.G.187 | down | −1.00332871 | 2.33E−05 | P06239 |
| 39 | 39 | 0 | LCK | P06239-3 | wp071_SB1.G.181 | SB1.G.181 | down | −1.77424988 | 1.56E−06 | P06239 |
| 39 | 39 | 0 | LCK | P06239-3 | wp071_SB1.G.192.1 | SB1.G.192.1 | down | −1.76709513 | 1.59E−06 | P06239 |
| 39 | 39 | 0 | LCK | P06239-3 | wp071_SB1.G.200 | SB1.G.200 | down | −1.27482599 | 7.84E−06 | P06239 |
| 39 | 39 | 0 | LCK | P06239-3 | wp074_dRAF.3 | dRAF.3 | down | −0.51294457 | 0.00238244 | P06239 |
| 39 | 39 | 0 | LCK | P06239-3 | wp103_DB0646 | DB0646 | down | −0.44960701 | 0.00010702 | P06239 |
| 39 | 39 | 0 | LCK | P06239-3 | wp103_SB1.G.187 | SB1.G.187 | down | −1.23747593 | 1.06E−06 | P06239 |
| 39 | 39 | 0 | LCK | P06239-3 | wp103_SK.3.91 | SK.3.91 | down | −1.14807457 | 1.50E−06 | P06239 |
| 39 | 39 | 0 | LCK | P06239-3 | wp107_DB0646 | DB0646 | down | −0.39630089 | 0.00046926 | P06239 |
| 39 | 39 | 0 | LCK | P06239-3 | wp107_SB1.G.187 | SB1.G.187 | down | −1.23701476 | 9.33E−08 | P06239 |
| 39 | 39 | 0 | LCK | P06239-3 | wp107_SK.3.91 | SK.3.91 | down | −1.57497865 | 3.89E−07 | P06239 |
| 39 | 39 | 0 | LCK | P06239-3 | wp117_DB0646 | DB0646 | down | −0.42022546 | 5.63E−06 | P06239 |
| 39 | 39 | 0 | LCK | P06239-3 | wp131_SB1.G.192.1 | SB1.G.192.1 | down | −1.70082449 | 7.30E−10 | P06239 |
| 39 | 39 | 0 | LCK | P06239-3 | wp131_TL12.186 | TL12.186 | down | −1.45495497 | 3.40E−09 | P06239 |
| 39 | 39 | 0 | LCK | P06239-3 | wp133_SK.3.91_2 hr | SK.3.91 | down | −0.53516742 | 5.88E−07 | P06239 |
| 39 | 39 | 0 | LCK | P06239-3 | wp133_SK.3.91_4 hr | SK.3.91 | down | −1.27685897 | 3.35E−09 | P06239 |
| 39 | 39 | 0 | LCK | P06239-3 | wp137_TL12.186 | TL12.186 | down | −1.37259986 | 2.06E−08 | P06239 |
| 39 | 39 | 0 | LCK | P06239-3 | wp137_TL13.97 | TL13.97 | down | −0.87210043 | 2.58E−07 | P06239 |
| 39 | 39 | 0 | LCK | P06239-1 | wp081_SB1.G.192.1 | SB1.G.192.1 | down | −1.74805301 | 4.51E−09 | P06239 |
| 39 | 39 | 0 | LCK | P06239-1 | wp081_TL13.150 | TL13.150 | down | −0.71291826 | 6.32E−07 | P06239 |
| 39 | 39 | 0 | LCK | P06239-1 | wp160_DB0646_4 hr | DB0646 | down | −0.45754431 | 3.68E−05 | P06239 |
| 39 | 39 | 0 | LCK | P06239-1 | wp160_DB0646_8 hr | DB0646 | down | −0.53858401 | 1.91E−05 | P06239 |
| 39 | 39 | 0 | LCK | P06239-1 | wp160_SK.3.91_2 hr | SK.3.91 | down | −0.46266831 | 3.52E−05 | P06239 |
| 39 | 39 | 0 | LCK | P06239-1 | wp160_SK.3.91_4 hr | SK.3.91 | down | −1.17345066 | 8.16E−07 | P06239 |
| 39 | 39 | 0 | LCK | P06239-1 | wp168_DB0661 | DB0661 | down | −0.47174688 | 1.54E−06 | P06239 |
| 39 | 39 | 0 | LCK | P06239-1 | wp168_DB0662 | DB0662 | down | −0.9078331 | 2.32E−07 | P06239 |
| 39 | 39 | 0 | LCK | P06239 | wp152_DB0646 | DB0646 | down | −0.44682367 | 1.73E−06 | P06239 |
| 39 | 39 | 0 | LCK | P06239 | wp152_DB1114 | DB1114 | down | −0.55712912 | 5.14E−07 | P06239 |
| 37 | 36 | 1 | PTK2B | Q14289-2 | wp107_DB0646 | DB0646 | down | −2.48421844 | 3.50E−09 | Q14289 |
| 37 | 36 | 1 | PTK2B | Q14289-2 | wp107_SK.3.91 | SK.3.91 | down | −3.12639372 | 4.84E−08 | Q14289 |
| 37 | 36 | 1 | PTK2B | Q14289-2 | wp117_DB0614 | DB0614 | down | −1.20251435 | 8.20E−06 | Q14289 |
| 37 | 36 | 1 | PTK2B | Q14289-2 | wp117_DB0646 | DB0646 | down | −2.58313178 | 3.99E−08 | Q14289 |
| 37 | 36 | 1 | PTK2B | Q14289-2 | wp168_DB0661 | DB0661 | down | −1.93549532 | 1.19E−09 | Q14289 |
| 37 | 36 | 1 | PTK2B | Q14289-2 | wp168_DB0662 | DB0662 | down | −3.24452302 | 1.79E−07 | Q14289 |
| 37 | 36 | 1 | PTK2B | Q14289-2 | wp168_DB1113 | DB1113 | down | −1.70519414 | 1.31E−09 | Q14289 |
| 37 | 36 | 1 | PTK2B | Q14289-1 | wp016_LT2.49 | LT2.49 | down | −3.36524391 | 4.44E−07 | Q14289 |
| 37 | 36 | 1 | PTK2B | Q14289-1 | wp016_SK.3.87 | SK.3.87 | down | −3.21731515 | 5.40E−07 | Q14289 |
| 37 | 36 | 1 | PTK2B | Q14289-1 | wp016_SK.3.89 | SK.3.89 | down | −3.55425196 | 3.50E−07 | Q14289 |
| 37 | 36 | 1 | PTK2B | Q14289-1 | wp016_SK.3.91 | SK.3.91 | down | −3.82375232 | 2.54E−07 | Q14289 |
| 37 | 36 | 1 | PTK2B | Q14289-1 | wp016_SK.3.91_1 uM | SK.3.91 | down | −3.60334947 | 3.29E−07 | Q14289 |
| 37 | 36 | 1 | PTK2B | Q14289-1 | wp016_SK.3.93 | SK.3.93 | down | −3.26949775 | 5.03E−07 | Q14289 |
| 37 | 36 | 1 | PTK2B | Q14289-1 | wp016_TL12.186 | TL12.186 | down | −3.72197602 | 2.86E−07 | Q14289 |
| 37 | 36 | 1 | PTK2B | Q14289-1 | wp045_DB0614 | DB0614 | down | −1.42536634 | 1.23E−06 | Q14289 |
| 37 | 36 | 1 | PTK2B | Q14289-1 | wp045_DB0646 | DB0646 | down | −2.77426078 | 6.56E−08 | Q14289 |
| 37 | 36 | 1 | PTK2B | Q14289-1 | wp045_DB0663 | DB0663 | down | −1.960653 | 3.02E−07 | Q14289 |
| 37 | 36 | 1 | PTK2B | Q14289-1 | wp045_RSS0628 | RSS0628 | down | −1.51684785 | 9.34E−07 | Q14289 |
| 37 | 36 | 1 | PTK2B | Q14289-1 | wp076_MFH.5.103.1 | MFH.5.103.1 | down | −3.17957002 | 4.15E−05 | Q14289 |
| 37 | 36 | 1 | PTK2B | Q14289-1 | wp076_MFH.5.116.1 | MFH.5.116.1 | down | −2.9463713 | 5.79E−05 | Q14289 |
| 37 | 36 | 1 | PTK2B | Q14289-1 | wp114_DB0646 | DB0646 | down | −1.35812195 | 3.40E−05 | Q14289 |
| 37 | 36 | 1 | PTK2B | Q14289-1 | wp114_SK.3.91 | SK.3.91 | down | −1.88466866 | 9.02E−06 | Q14289 |
| 37 | 36 | 1 | PTK2B | Q14289-1 | wp133_SK.3.91_1 hr | SK.3.91 | down | −0.80557608 | 0.00014767 | Q14289 |
| 37 | 36 | 1 | PTK2B | Q14289-1 | wp133_SK.3.91_2 hr | SK.3.91 | down | −2.02674104 | 7.71E−07 | Q14289 |
| 37 | 36 | 1 | PTK2B | Q14289-1 | wp133_SK.3.91_4 hr | SK.3.91 | down | −3.95969674 | 2.29E−07 | Q14289 |
| 37 | 36 | 1 | PTK2B | Q14289-1 | wp146_TMX.02.138 | TMX.02.138 | down | −1.23965225 | 0.00108228 | Q14289 |
| 37 | 36 | 1 | PTK2B | Q14289-1 | wp146_TMX.02.174 | TMX.02.174 | down | −1.51219009 | 0.00053274 | Q14289 |
| 37 | 36 | 1 | PTK2B | Q14289-1 | wp160_DB0646_1 hr | DB0646 | down | −0.39548679 | 0.00038973 | Q14289 |
| 37 | 36 | 1 | PTK2B | Q14289-1 | wp160_DB0646_2 hr | DB0646 | down | −1.23545784 | 4.03E−06 | Q14289 |
| 37 | 36 | 1 | PTK2B | Q14289-1 | wp160_DB0646_4 hr | DB0646 | down | −2.34108139 | 3.02E−07 | Q14289 |
| 37 | 36 | 1 | PTK2B | Q14289-1 | wp160_DB0646_8 hr | DB0646 | down | −2.59943781 | 1.97E−07 | Q14289 |
| 37 | 36 | 1 | PTK2B | Q14289-1 | wp160_SK.3.91_1 hr | SK.3.91 | down | −1.04349129 | 7.98E−06 | Q14289 |
| 37 | 36 | 1 | PTK2B | Q14289-1 | wp160_SK.3.91_2 hr | SK.3.91 | down | −2.10471453 | 4.65E−07 | Q14289 |
| 37 | 36 | 1 | PTK2B | Q14289-1 | wp160_SK.3.91_4 hr | SK.3.91 | down | −2.77294534 | 1.52E−07 | Q14289 |
| 37 | 36 | 1 | PTK2B | Q14289 | wp152_DB0646 | DB0646 | down | −2.99783418 | 2.42E−10 | Q14289 |
| 37 | 36 | 1 | PTK2B | Q14289 | wp152_DB1114 | DB1114 | down | −2.03854035 | 2.05E−09 | Q14289 |
| 26 | 26 | 0 | MAPK14 | Q16539-2 | wp047_DD.02.198 | DD.02.198 | down | −0.61696537 | 0.00215271 | Q16539 |
| 26 | 26 | 0 | MAPK14 | Q16539-2 | wp056_SB1.G.187 | SB1.G.187 | down | −0.80194366 | 0.00982693 | Q16539 |
| 26 | 26 | 0 | MAPK14 | Q16539-2 | wp081_TL13.150 | TL13.150 | down | −0.50248382 | 0.0006474 | Q16539 |
| 26 | 26 | 0 | MAPK14 | Q16539-2 | wp081_TL13.178 | TL13.178 | down | −0.33425976 | 0.00458601 | Q16539 |
| 26 | 26 | 0 | MAPK14 | Q16539-2 | wp105_DB0646 | DB0646 | down | −0.58218857 | 0.00438114 | Q16539 |
| 26 | 26 | 0 | MAPK14 | Q16539-2 | wp105_SB1.G.187 | SB1.G.187 | down | −0.9083618 | 0.00054846 | Q16539 |
| 26 | 26 | 0 | MAPK14 | Q16539-2 | wp117_DB0646 | DB0646 | down | −0.64360505 | 0.00339306 | Q16539 |
| 26 | 26 | 0 | MAPK14 | Q16539-2 | wp160_DB0846_4 hr | DB0646 | down | −0.70507836 | 0.001463 | Q16539 |
| 26 | 26 | 0 | MAPK14 | Q16539-2 | wp160_DB0646_8 hr | DB0646 | down | −1.09189072 | 0.00026449 | Q16539 |
| 26 | 26 | 0 | MAPK14 | Q16539-2 | wp168_DB0662 | DB0662 | down | −1.33952494 | 5.07E−05 | Q16539 |
| 26 | 26 | 0 | MAPK14 | Q16539-2 | wp168_DB1113 | DB1113 | down | −1.58778653 | 1.72E−05 | Q16539 |
| 26 | 26 | 0 | MAPK14 | Q16539 | wp045_DB0646 | DB0646 | down | −0.77930772 | 0.00011604 | Q16539 |
| 26 | 26 | 0 | MAPK14 | Q16539 | wp045_DB0663 | DB0663 | down | −0.62929522 | 0.00029009 | Q16539 |
| 26 | 26 | 0 | MAPK14 | Q16539 | wp047_DD.02.198 | DD.02.198 | down | −1.38729618 | 8.76E−06 | Q16539 |
| 26 | 26 | 0 | MAPK14 | Q16539 | wp071_SB1.G.192.1 | SB1.G.192.1 | down | −0.89149398 | 4.75E−05 | Q16539 |
| 26 | 26 | 0 | MAPK14 | Q16539 | wp081_SB1.G.192.1 | SB1.G.192.1 | down | −0.72927081 | 3.60E−05 | Q16539 |
| 26 | 26 | 0 | MAPK14 | Q16539 | wp081_TL13.150 | TL13.150 | down | −0.37382697 | 0.00114461 | Q16539 |
| 26 | 26 | 0 | MAPK14 | Q16539 | wp081_TL13.178 | TL13.178 | down | −0.5658529 | 0.00013826 | Q16539 |
| 26 | 26 | 0 | MAPK14 | Q16539 | wp103_DB0646 | DB0646 | down | −0.60130881 | 0.00056976 | Q16539 |
| 26 | 26 | 0 | MAPK14 | Q16539 | wp103_SB1.G.187 | SB1.G.187 | down | −0.8300171 | 0.00013677 | Q16539 |
| 26 | 26 | 0 | MAPK14 | Q16539 | wp107_DB0646 | DB0646 | down | −0.76008968 | 5.42E−05 | Q16539 |
| 26 | 26 | 0 | MAPK14 | Q16539 | wp107_SB1.G.187 | SB1.G.187 | down | −0.92395051 | 7.26E−06 | Q16539 |
| 26 | 26 | 0 | MAPK14 | Q16539 | wp114_DB0646 | DB0646 | down | −0.58138084 | 0.00867346 | Q16539 |
| 26 | 26 | 0 | MAPK14 | Q16539 | wp117_DB0646 | DB0646 | down | −0.63919941 | 0.00017943 | Q16539 |
| 26 | 26 | 0 | MAPK14 | Q16539 | wp131_SB1.G.192.1 | SB1.G.192.1 | down | −0.74631065 | 1.19E−06 | Q16539 |
| 26 | 26 | 0 | MAPK14 | Q16539 | wp152_DB0646 | DB0646 | down | −0.78207982 | 6.16E−08 | Q16539 |
| 26 | 26 | 0 | MAPK14 | Q16539 | wp152_DB1114 | DB1114 | down | −1.39886922 | 2.47E−09 | Q16539 |
| 26 | 26 | 0 | MAPK14 | Q16539 | wp160_DB0646_2 hr | DB0646 | down | −0.44041115 | 0.00017285 | Q16539 |
| 26 | 26 | 0 | MAPK14 | Q16539 | wp160_DB0646_4 hr | DB0646 | down | −0.71619543 | 2.46E−05 | Q16539 |
| 26 | 26 | 0 | MAPK14 | Q16539 | wp160_DB0646_8 hr | DB0646 | down | −1.15321629 | 3.59E−06 | Q16539 |
| 26 | 26 | 0 | MAPK14 | Q16539 | wp166_DB0662 | DB0662 | down | −1.00173673 | 1.19E−05 | Q16539 |
| 26 | 26 | 0 | MAPK14 | Q16539 | wp168_DB1113 | DB1113 | down | −1.73012312 | 6.08E−08 | Q16539 |
| 26 | 26 | 0 | MAPK14 | Q16539 | wp172_DB0646 | DB0646 | down | −0.64689544 | 3.21E−05 | Q16539 |
| 26 | 26 | 0 | MAPK14 | Q16539 | wp172_SB1.G.187 | SB1.G.187 | down | −0.51976198 | 0.00021888 | Q16539 |
| 34 | 34 | 0 | CSK | P41240 | wp016_SK.3.91 | SK.3.91 | down | −0.32539106 | 0.00090638 | P41240 |
| 34 | 34 | 0 | CSK | P41240 | wp016_SK.3.91_1 uM | SK.3.91 | down | −0.65956347 | 4.54E−05 | P41240 |
| 34 | 34 | 0 | CSK | P41240 | wp045_DB.3.291 | DB.3.291 | down | −2.25671146 | 1.04E−08 | P41240 |
| 34 | 34 | 0 | CSK | P41240 | wp045_DB0646 | DB0646 | down | −1.54171198 | 5.55E−08 | P41240 |
| 34 | 34 | 0 | CSK | P41240 | wp045_DB0663 | DB0663 | down | −1.20613344 | 1.64E−07 | P41240 |
| 34 | 34 | 0 | CSK | P41240 | wp047_DD.03.119 | DD.03.119 | down | −1.78614803 | 4.51E−08 | P41240 |
| 34 | 34 | 0 | CSK | P41240 | wp071_FMF.05.178.1 | FMF.05.178.1 | down | −0.4779011 | 5.94E−05 | P41240 |
| 34 | 34 | 0 | CSK | P41240 | wp071_SB1.G.181 | SB1.G.181 | down | −3.22938864 | 5.24E−09 | P41240 |
| 34 | 34 | 0 | CSK | P41240 | wp071_SB1.G.192.1 | SB1.G.192.1 | down | −2.27134479 | 2.95E−08 | P41240 |
| 34 | 34 | 0 | CSK | P41240 | wp071_SB1.G.200 | SB1.G.200 | down | −3.01105751 | 7.39E−09 | P41240 |
| 34 | 34 | 0 | CSK | P41240 | wp074_dRAF.3 | dRAF.3 | down | −2.0936583 | 3.48E−07 | P41240 |
| 34 | 34 | 0 | CSK | P41240 | wp081_SB1.G.192.1 | SB1.G.192.1 | down | −1.96586625 | 4.36E−10 | P41240 |
| 34 | 34 | 0 | CSK | P41240 | wp103_DB0646 | DB0646 | down | −1.28187721 | 8.40E−07 | P41240 |
| 34 | 34 | 0 | CSK | P41240 | wp103_SK.3.91 | SK.3.91 | down | −0.43429266 | 0.00011623 | P41240 |
| 34 | 34 | 0 | CSK | P41240 | wp105_DB0646 | DB0646 | down | −0.68677249 | 9.90E−07 | P41240 |
| 34 | 34 | 0 | CSK | P41240 | wp105_SK.3.91 | SK.3.91 | down | −0.70436502 | 7.51E−07 | P41240 |
| 34 | 34 | 0 | CSK | P41240 | wp107_DB0646 | DB0646 | down | −1.31123708 | 5.67E−08 | P41240 |
| 34 | 34 | 0 | CSK | P41240 | wp107_SK.3.91 | SK.3.91 | down | −0.5646008 | 1.18E−06 | P41240 |
| 34 | 34 | 0 | CSK | P41240 | wp114_DB0646 | DB0646 | down | −0.6931505 | 3.21E−05 | P41240 |
| 34 | 34 | 0 | CSK | P41240 | wp117_DB0646 | DB0646 | down | −1.41371096 | 6.97E−08 | P41240 |
| 34 | 34 | 0 | CSK | P41240 | wp131_SB1.G.192.1 | SB1.G.192.1 | down | −1.81603198 | 1.61E−09 | P41240 |
| 34 | 34 | 0 | CSK | P41240 | wp133_SK.3.91_4 hr | SK.3.91 | down | −0.5491765 | 2.12E−07 | P41240 |
| 34 | 34 | 0 | CSK | P41240 | wp147_DD.03.106_4 uM | DD.03.106 | down | −0.51493628 | 6.82E−06 | P41240 |
| 34 | 34 | 0 | CSK | P41240 | wp152_DB0646 | DB0646 | down | −1.51303531 | 1.80E−09 | P41240 |
| 34 | 34 | 0 | CSK | P41240 | wp152_DB1114 | DB1114 | down | −1.52056954 | 1.75E−09 | P41240 |
| 34 | 34 | 0 | CSK | P41240 | wp160_DB0646_2 hr | DB0646 | down | −0.83076977 | 1.38E−05 | P41240 |
| 34 | 34 | 0 | CSK | P41240 | wp160_DB0646_4 hr | DB0646 | down | −1.41641025 | 1.59E−06 | P41240 |
| 34 | 34 | 0 | CSK | P41240 | wp160_DB0646_8 hr | DB0646 | down | −1.75284551 | 6.70E−07 | P41240 |
| 34 | 34 | 0 | CSK | P41240 | wp160_SK.3.91_4 hr | SK.3.91 | down | −0.49764646 | 0.00010804 | P41240 |
| 34 | 34 | 0 | CSK | P41240 | wp168_DB0661 | DB0661 | down | −1.52609963 | 9.72E−08 | P41240 |
| 34 | 34 | 0 | CSK | P41240 | wp168_DB0662 | DB0662 | down | −1.83286123 | 2.86E−08 | P41240 |
| 34 | 34 | 0 | CSK | P41240 | wp168_DB1113 | DB1113 | down | −1.61999172 | 1.14E−07 | P41240 |
| 34 | 34 | 0 | CSK | P41240 | wp172_DB0646 | DB0646 | down | −0.85381208 | 4.56E−07 | P41240 |
| 34 | 34 | 0 | CSK | P41240 | wp172_SK.3.91 | SK.3.91 | down | −0.42248265 | 8.89E−06 | P41240 |
| 33 | 33 | 0 | SIK2 | Q9H0K1 | wp045_DB0614 | DB0614 | down | −0.52916806 | 0.00482292 | Q9H0K1 |
| 33 | 33 | 0 | SIK2 | Q9H0K1 | wp045_DB0646 | DB0646 | down | −0.63803693 | 0.00229682 | Q9H0K1 |
| 33 | 33 | 0 | SIK2 | Q9H0K1 | wp045_DB0663 | DB0663 | down | −0.47652187 | 0.0072119 | Q9H0K1 |
| 33 | 33 | 0 | SIK2 | Q9H0K1 | wp071_SB1.G.192.1 | SB1.G.192.1 | down | −1.03643959 | 0.00115491 | Q9H0K1 |
| 33 | 33 | 0 | SIK2 | Q9H0K1 | wp080_RSS0680 | RSS0680 | down | −0.69861558 | 0.0002511 | Q9H0K1 |
| 33 | 33 | 0 | SIK2 | Q9H0K1 | wp081_SB1.G.192.1 | SB1.G.192.1 | down | −1.19554179 | 0.00075419 | Q9H0K1 |
| 33 | 33 | 0 | SIK2 | Q9H0K1 | wp105_SK.3.91 | SK.3.91 | down | −0.53861402 | 0.00042348 | Q9H0K1 |
| 33 | 33 | 0 | SIK2 | Q9H0K1 | wp107_DB0646 | DB0646 | down | −0.59326952 | 0.00340048 | Q9H0K1 |
| 33 | 33 | 0 | SIK2 | Q9H0K1 | wp107_SK.3.91 | SK.3.91 | down | −1.5680839 | 4.80E−05 | Q9H0K1 |
| 33 | 33 | 0 | SIK2 | Q9H0K1 | wp114_DB0646 | DB0646 | down | −0.43467357 | 0.00410763 | Q9H0K1 |
| 33 | 33 | 0 | SIK2 | Q9H0K1 | wp114_FMF.06.098.1 | FMF.06.098.1 | down | −0.49847091 | 0.00245717 | Q9H0K1 |
| 33 | 33 | 0 | SIK2 | Q9H0K1 | wp114_SK.3.91 | SK.3.91 | down | −0.57565063 | 0.00141651 | Q9H0K1 |
| 33 | 33 | 0 | SIK2 | Q9H0K1 | wp117_DB0614 | DB0614 | down | −0.37655222 | 0.00066369 | Q9H0K1 |
| 33 | 33 | 0 | SIK2 | Q9H0K1 | wp117_DB0646 | DB0646 | down | −0.43151179 | 0.00042948 | Q9H0K1 |
| 33 | 33 | 0 | SIK2 | Q9H0K1 | wp117_RSS0680 | RSS0680 | down | −0.7184217 | 7.28E−05 | Q9H0K1 |
| 33 | 33 | 0 | SIK2 | Q9H0K1 | wp131_FMF.06.098.1 | FMF.06.098.1 | down | −0.60202062 | 2.09E−05 | Q9H0K1 |
| 33 | 33 | 0 | SIK2 | Q9H0K1 | wp131_SB1.G.192.1 | SB1.G.192.1 | down | −0.93416819 | 2.90E−06 | Q9H0K1 |
| 33 | 33 | 0 | SIK2 | Q9H0K1 | wp131_TL12.186 | TL12.186 | down | −1.31693145 | 2.75E−07 | Q9H0K1 |
| 33 | 33 | 0 | SIK2 | Q9H0K1 | wp133_SK.3.91_2 hr | SK.3.91 | down | −0.49380583 | 2.09E−05 | Q9H0K1 |
| 33 | 33 | 0 | SIK2 | Q9H0K1 | wp133_SK.3.91_4 hr | SK.3.91 | down | −1.0345184 | 2.74E−07 | Q9H0K1 |
| 33 | 33 | 0 | SIK2 | Q9H0K1 | wp134_BS.J.04.178 | BSJ.04.178 | down | −1.35112227 | 9.15E−07 | Q9H0K1 |
| 33 | 33 | 0 | SIK2 | Q9H0K1 | wp134_DB118430 | DB118430 | down | −0.38028962 | 0.00103817 | Q9H0K1 |
| 33 | 33 | 0 | SIK2 | Q9H0K1 | wp152_DB1114 | DB1114 | down | −0.54248487 | 0.00543813 | Q9H0K1 |
| 33 | 33 | 0 | SIK2 | Q9H0K1 | wp160_DB0646_4 hr | DB0646 | down | −0.54210032 | 0.00331251 | Q9H0K1 |
| 33 | 33 | 0 | SIK2 | Q9H0K1 | wp160_DB0646_8 hr | DB0646 | down | −0.57072965 | 0.00273143 | Q9H0K1 |
| 33 | 33 | 0 | SIK2 | Q9H0K1 | wp160_SK.3.91_2 hr | SK.3.91 | down | −0.54126933 | 0.00333153 | Q9H0K1 |
| 33 | 33 | 0 | SIK2 | Q9H0K1 | wp160_SK.3.91_4 hr | SK.3.91 | down | −0.83639433 | 0.00062786 | Q9H0K1 |
| 33 | 33 | 0 | SIK2 | Q9H0K1 | wp168_DB0661 | DB0661 | down | −0.36284727 | 0.00079208 | Q9H0K1 |
| 33 | 33 | 0 | SIK2 | Q9H0K1 | wp168_DB0662 | DB0662 | down | −0.77699595 | 1.45E−05 | Q9H0K1 |
| 33 | 33 | 0 | SIK2 | Q9H0K1 | wp168_DB1113 | DB1113 | down | −0.7295827 | 2.32E−05 | Q9H0K1 |
| 33 | 33 | 0 | SIK2 | Q9H0K1 | wp195_BSJ.05.026 | BSJ.05.026 | down | −0.94719792 | 1.78E−06 | Q9H0K1 |
| 33 | 33 | 0 | SIK2 | Q9H0K1 | wp195_JWG.123 | JWG.123 | down | −0.61426972 | 1.93E−05 | Q9H0K1 |
| 33 | 33 | 0 | SIK2 | Q9H0K1 | wp195_JWG.148 | JWG.148 | down | −0.45496551 | 9.75E−05 | Q9H0K1 |
| 33 | 33 | 0 | SRC | P12931-2 | wp016_SK.3.91 | SK.3.91 | down | −0.38465614 | 0.00258585 | P12931 |
| 33 | 33 | 0 | SRC | P12931-2 | wp016_SK.3.91_1 uM | SK.3.91 | down | −0.84279894 | 9.84E−05 | P12931 |
| 33 | 33 | 0 | SRC | P12931-2 | wp016_SK.3.93 | SK.3.93 | down | −0.34558091 | 0.00394768 | P12931 |
| 33 | 33 | 0 | SRC | P12931-2 | wp016_TL12.186 | TL12.186 | down | −0.35729806 | 0.00346379 | P12931 |
| 33 | 33 | 0 | SRC | P12931-2 | wp045_DB0646 | DB0646 | down | −0.41522938 | 0.00046895 | P12931 |
| 33 | 33 | 0 | SRC | P12931-2 | wp045_DB0663 | DB0663 | down | −0.42467873 | 0.0004264 | P12931 |
| 33 | 33 | 0 | SRC | P12931-2 | wp047_DD.03.119 | DD.03.119 | down | −0.61278382 | 4.00E−05 | P12931 |
| 33 | 33 | 0 | SRC | P12931-2 | wp056_SB1.G.187 | SB1.G.187 | down | −0.60401052 | 0.00016278 | P12931 |
| 33 | 33 | 0 | SRC | P12931-2 | wp071_SB1.G.181 | SB1.G.181 | down | −2.47795441 | 4.50E−07 | P12931 |
| 33 | 33 | 0 | SRC | P12931-2 | wp071_SB1.G.192.1 | SB1.G.192.1 | down | −2.57828298 | 3.70E−07 | P12931 |
| 33 | 33 | 0 | SRC | P12931-2 | wp071_SB1.G.200 | SB1.G.200 | down | −2.17429995 | 8.54E−07 | P12931 |
| 33 | 33 | 0 | SRC | P12931-2 | wp081_SB1.G.192.1 | SB1.G.192.1 | down | −2.11298079 | 4.41E−09 | P12931 |
| 33 | 33 | 0 | SRC | P12931-2 | wp081_TL13.150 | TL13.150 | down | −0.53308341 | 8.53E−06 | P12931 |
| 33 | 33 | 0 | SRC | P12931-2 | wp103_SB1.G.187 | SB1.G.187 | down | −0.82821901 | 7.79E−05 | P12931 |
| 33 | 33 | 0 | SRC | P12931-2 | wp103_SK.3.91 | SK.3.91 | down | −0.50037839 | 0.0007275 | P12931 |
| 33 | 33 | 0 | SRC | P12931-2 | wp105_DB0646 | DB0646 | down | −0.44123717 | 0.00013959 | P12931 |
| 33 | 33 | 0 | SRC | P12931-2 | wp105_SB1.G.187 | SB1.G.187 | down | −0.56830627 | 1.12E−05 | P12931 |
| 33 | 33 | 0 | SRC | P12931-2 | wp105_SK.3.91 | SK.3.91 | down | −0.3464223 | 0.00011493 | P12931 |
| 33 | 33 | 0 | SRC | P12931-2 | wp107_DB0646 | DB0646 | down | −0.42743434 | 8.93E−06 | P12931 |
| 33 | 33 | 0 | SRC | P12931-2 | wp107_SB1.G.187 | SB1.G.187 | down | −1.20582318 | 8.13E−08 | P12931 |
| 33 | 33 | 0 | SRC | P12931-2 | wp107_SK.3.91 | SK.3.91 | down | −0.77820315 | 1.22E−06 | P12931 |
| 33 | 33 | 0 | SRC | P12931-2 | wp117_DB0646 | DB0646 | down | −0.38392134 | 0.00104371 | P12931 |
| 33 | 33 | 0 | SRC | P12931-2 | wp131_SB1.G.192.1 | SB1.G.192.1 | down | −2.12659781 | 2.85E−08 | P12931 |
| 33 | 33 | 0 | SRC | P12931-2 | wp131_TL12.186 | TL12.186 | down | −0.77772062 | 7.48E−06 | P12931 |
| 33 | 33 | 0 | SRC | P12931-2 | wp133_SK.3.91_4 hr | SK.3.91 | down | −0.64075559 | 6.71E−07 | P12931 |
| 33 | 33 | 0 | SRC | P12931-2 | wp137_TL12.186 | TL12.186 | down | −0.57378808 | 5.24E−05 | P12931 |
| 33 | 33 | 0 | SRC | P12931-2 | wp137_TL13.97 | TL13.97 | down | −0.48243123 | 0.00013216 | P12931 |
| 33 | 33 | 0 | SRC | P12931 | wp152_DB0646 | DB0646 | down | −0.39405599 | 0.00039992 | P12931 |
| 33 | 33 | 0 | SRC | P12931 | wp160_DB0646_4 hr | DB0646 | down | −0.51856115 | 3.62E−05 | P12931 |
| 33 | 33 | 0 | SRC | P12931 | wp160_DB0646_8 hr | DB0646 | down | −0.6832844 | 1.19E−05 | P12931 |
| 33 | 33 | 0 | SRC | P12931 | wp160_SK.3.91_4 hr | SK.3.91 | down | −0.52917934 | 3.33E−05 | P12931 |
| 33 | 33 | 0 | SRC | P12931 | wp168_DB0661 | DB0661 | down | −0.66849148 | 6.71E−06 | P12931 |
| 33 | 33 | 0 | SRC | P12931 | wp168_DB0662 | DB0662 | down | −0.90179573 | 1.40E−06 | P12931 |
| 32 | 32 | 0 | CDK17 | Q00537-2 | wp137_DD.03.106 | DD.03.106 | down | −0.86815949 | 6.86E−06 | Q00537 |
| 32 | 32 | 0 | CDK17 | Q00537-2 | wp137_TL12.186 | TL12.186 | down | −0.70263341 | 2.18E−05 | Q00537 |
| 32 | 32 | 0 | CDK17 | Q00537-2 | wp137_TL13.97 | TL13.97 | down | −0.56829347 | 6.85E−05 | Q00537 |
| 32 | 32 | 0 | CDK17 | Q00537 | wp016_LT2.49 | LT2.49 | down | −0.75444989 | 0.0004084 | Q00537 |
| 32 | 32 | 0 | CDK17 | Q00537 | wp016_SK.3.87 | SK.3.87 | down | −0.6139148 | 0.00096395 | Q00537 |
| 32 | 32 | 0 | CDK17 | Q00537 | wp016_SK.3.89 | SK.3.89 | down | −0.60565069 | 0.00101932 | Q00537 |
| 32 | 32 | 0 | CDK17 | Q00537 | wp016_SK.3.91 | SK.3.91 | down | −0.73407877 | 0.00045814 | Q00537 |
| 32 | 32 | 0 | CDK17 | Q00537 | wp016_SK.3.91_1 uM | SK.3.91 | down | −0.75032875 | 0.00041791 | Q00537 |
| 32 | 32 | 0 | CDK17 | Q00537 | wp016_SK.3.93 | SK.3.93 | down | −0.71026483 | 0.000526 | Q00537 |
| 32 | 32 | 0 | CDK17 | Q00537 | wp016_TL12.186 | TL12.186 | down | −0.84003685 | 0.00025954 | Q00537 |
| 32 | 32 | 0 | CDK17 | Q00537 | wp047_DD.03.106 | DD.03.106 | down | −0.72001172 | 0.00011196 | Q00537 |
| 32 | 32 | 0 | CDK17 | Q00537 | wp056_DD.03.156 | DD.03.156 | down | −1.82489783 | 5.89E−06 | Q00537 |
| 32 | 32 | 0 | CDK17 | Q00537 | wp076_MFH.5.126.1 | MFH.5.126.1 | down | −0.58944916 | 0.00213895 | Q00537 |
| 32 | 32 | 0 | CDK17 | Q00537 | wp080_RSS0680 | RSS0680 | down | −0.787504 | 4.14E−05 | Q00537 |
| 32 | 32 | 0 | CDK17 | Q00537 | wp103_SK.3.91 | SK.3.91 | down | −0.60153576 | 0.0013029 | Q00537 |
| 32 | 32 | 0 | CDK17 | Q00537 | wp104_TMX.01.160.1_0.25 | TMX.01.160.1 | down | −0.77881371 | 9.19E−05 | Q00537 |
| 32 | 32 | 0 | CDK17 | Q00537 | wp104_TMX.01.160.1_1 | TMX.01.160.1 | down | −0.68174561 | 0.00017722 | Q00537 |
| 32 | 32 | 0 | CDK17 | Q00537 | wp105_SK.3.91 | SK.3.91 | down | −0.73199922 | 0.00010479 | Q00537 |
| 32 | 32 | 0 | CDK17 | Q00537 | wp107_SK.3.91 | SK.3.91 | down | −0.61448647 | 4.84E−06 | Q00537 |
| 32 | 32 | 0 | CDK17 | Q00537 | wp117_RSS0680 | RSS0680 | down | −0.71932231 | 3.80E−06 | Q00537 |
| 32 | 32 | 0 | CDK17 | Q00537 | wp131_TL12.186 | TL12.186 | down | −0.61653906 | 4.01E−06 | Q00537 |
| 32 | 32 | 0 | CDK17 | Q00537 | wp133_SK.3.91_2 hr | SK.3.91 | down | −0.37195541 | 2.99E−05 | Q00537 |
| 32 | 32 | 0 | CDK17 | Q00537 | wp133_SK.3.91_4 hr | SK.3.91 | down | −0.61101717 | 1.15E−07 | Q00537 |
| 32 | 32 | 0 | CDK17 | Q00537 | wp146_TMX.01.160 | TMX.01.160 | down | −1.62662987 | 7.99E−05 | Q00537 |
| 32 | 32 | 0 | CDK17 | Q00537 | wp146_TMX.02.138 | TMX.02.138 | down | −1.36106088 | 0.00015263 | Q00537 |
| 32 | 32 | 0 | CDK17 | Q00537 | wp146_TMX.02.174 | TMX.02.174 | down | −1.41955288 | 0.00013103 | Q00537 |
| 32 | 32 | 0 | CDK17 | Q00537 | wp147_DD.03.106_2 uM | DD.03.108 | down | −0.8288227 | 9.56E−06 | Q00537 |
| 32 | 32 | 0 | CDK17 | Q00537 | wp147_DD.03.106_1 uM | DD.03.106 | down | −0.76149611 | 1.47E−05 | Q00537 |
| 32 | 32 | 0 | CDK17 | Q00537 | wp147_DD.03.106_4 uM | DD.03.106 | down | −0.62316951 | 4.05E−05 | Q00537 |
| 32 | 32 | 0 | CDK17 | Q00537 | wp160_SK.3.91_4 hr | SK.3.91 | down | −0.39520729 | 0.00227548 | Q00537 |
| 32 | 32 | 0 | CDK17 | Q00537 | wp172_SB1.G.187 | SB1.G.187 | down | −0.35620722 | 0.00610798 | Q00537 |
| 32 | 32 | 0 | CDK17 | Q00537 | wp172_SK.3.91 | SK.3.91 | down | −0.74915419 | 4.17E−05 | Q00537 |
| 32 | 32 | 0 | CDK5 | Q00535 | wp016_LT2.49 | LT2.49 | down | −0.86083532 | 1.48E−05 | Q00535 |
| 32 | 32 | 0 | CDK5 | Q00535 | wp016_SK.3.87 | SK.3.87 | down | −0.57318381 | 8.52E−05 | Q00535 |
| 32 | 32 | 0 | CDK5 | Q00535 | wp016_SK.3.89 | SK.3.89 | down | −0.62543639 | 5.86E−05 | Q00535 |
| 32 | 32 | 0 | CDK5 | Q00535 | wp016_SK.3.91 | SK.3.91 | down | −1.12617994 | 4.60E−06 | Q00535 |
| 32 | 32 | 0 | CDK5 | Q00535 | wp016_SK.3.91_1 uM | SK.3.91 | down | −1.42877606 | 1.63E−06 | Q00535 |
| 32 | 32 | 0 | CDK5 | Q00535 | wp016_SK.3.93 | SK.3.93 | down | −0.97025994 | 8.79E−06 | Q00535 |
| 32 | 32 | 0 | CDK5 | Q00535 | wp016_TL12.186 | TL12.186 | down | −0.92608197 | 1.08E−05 | Q00535 |
| 32 | 32 | 0 | CDK5 | Q00535 | wp056_SB1.G.187 | SB1.G.187 | down | −0.4803833 | 0.00276288 | Q00535 |
| 32 | 32 | 0 | CDK5 | Q00535 | wp081_TL13.150 | TL13.150 | down | −0.37863826 | 9.35E−06 | Q00535 |
| 32 | 32 | 0 | CDK5 | Q00535 | wp103_SB1.G.187 | SB1.G.187 | down | −0.59240625 | 0.00012964 | Q00535 |
| 32 | 32 | 0 | CDK5 | Q00535 | wp103_SK.3.91 | SK.3.91 | down | −1.0431215 | 1.00E−05 | Q00535 |
| 32 | 32 | 0 | CDK5 | Q00535 | wp104_TMX.01.160.1_0.25 | TMX.01.160.1 | down | −1.86630681 | 1.48E−07 | Q00535 |
| 32 | 32 | 0 | CDK5 | Q00535 | wp104_TMX.01.160.1_1 | TMX.01.160.1 | down | −1.8267937 | 1.65E−07 | Q00535 |
| 32 | 32 | 0 | CDK5 | Q00535 | wp105_SB1.G.187 | SB1.G.187 | down | −0.60527761 | 1.36E−05 | Q00535 |
| 32 | 32 | 0 | CDK5 | Q00535 | wp105_SK.3.91 | SK.3.91 | down | −0.93104014 | 7.01E−07 | Q00535 |
| 32 | 32 | 0 | CDK5 | Q00535 | wp107_SB1.G.187 | SB1.G.187 | down | −0.67779393 | 1.41E−05 | Q00535 |
| 32 | 32 | 0 | CDK5 | Q00535 | wp107_SK.3.91 | SK.3.91 | down | −1.01448395 | 1.62E−06 | Q00535 |
| 32 | 32 | 0 | CDK5 | Q00535 | wp114_SK.3.91 | SK.3.91 | down | −0.46799686 | 0.0041429 | Q00535 |
| 32 | 32 | 0 | CDK5 | Q00535 | wp131_TL12.186 | TL12.186 | down | −1.30048145 | 1.67E−07 | Q00535 |
| 32 | 32 | 0 | CDK5 | Q00535 | wp133_SK.3.91_2 hr | SK.3.91 | down | −0.66077789 | 1.06E−06 | Q00535 |
| 32 | 32 | 0 | CDK5 | Q00535 | wp133_SK.3.91_4 hr | SK.3.91 | down | −1.24209567 | 1.33E−08 | Q00535 |
| 32 | 32 | 0 | CDK5 | Q00535 | wp137_TL12.186 | TL12.186 | down | −1.23410582 | 8.09E−09 | Q00535 |
| 32 | 32 | 0 | CDK5 | Q00535 | wp137_TL13.97 | TL13.97 | down | −1.11383271 | 1.43E−08 | Q00535 |
| 32 | 32 | 0 | CDK5 | Q00535 | wp146_TMX.01.160 | TMX.01.160 | down | −2.90117347 | 1.67E−06 | Q00535 |
| 32 | 32 | 0 | CDK5 | Q00535 | wp146_TMX.02.138 | TMX.02.138 | down | −3.28985281 | 1.06E−06 | Q00535 |
| 32 | 32 | 0 | CDK5 | Q00535 | wp146_TMX.02.172 | TMX.02.172 | down | −2.87788115 | 1.73E−06 | Q00535 |
| 32 | 32 | 0 | CDK5 | Q00535 | wp146_TMX.02.174 | TMX.02.174 | down | −3.16070484 | 1.22E−06 | Q00535 |
| 32 | 32 | 0 | CDK5 | Q00535 | wp160_SK.3.91_1 hr | SK.3.91 | down | −0.32572193 | 0.00080289 | Q00535 |
| 32 | 32 | 0 | CDK5 | Q00535 | wp160_SK.3.91_2 hr | SK.3.91 | down | −0.62645299 | 6.00E−05 | Q00535 |
| 32 | 32 | 0 | CDK5 | Q00535 | wp160_SK.3.91_4 hr | SK.3.91 | down | −1.19706388 | 4.40E−06 | Q00535 |
| 32 | 32 | 0 | CDK5 | Q00535 | wp172_SB1.G.187 | SB1.G.187 | down | −0.89384384 | 2.54E−06 | Q00535 |
| 32 | 32 | 0 | CDK5 | Q00535 | wp172_SK.3.91 | SK.3.91 | down | −1.30414715 | 1.08E−07 | Q00535 |
| 32 | 32 | 0 | CDK2 | P24941-1 | wp016_LT2.49 | LT2.49 | down | −0.51768964 | 0.00041814 | P24941 |
| 32 | 32 | 0 | CDK2 | P24941-1 | wp016_SK.3.87 | SK.3.87 | down | −0.37968344 | 0.00150997 | P24941 |
| 32 | 32 | 0 | CDK2 | P24941-1 | wp016_SK.3.89 | SK.3.89 | down | −0.59530763 | 0.00023184 | P24941 |
| 32 | 32 | 0 | CDK2 | P24941-1 | wp016_SK.3.91 | SK.3.91 | down | −0.51148242 | 0.00043986 | P24941 |
| 32 | 32 | 0 | CDK2 | P24941-1 | wp016_SK.3.91_1 uM | SK.3.91 | down | −1.04702416 | 2.06E−05 | P24941 |
| 32 | 32 | 0 | CDK2 | P24941-1 | wp016_SK.3.93 | SK.3.93 | down | −0.54330105 | 0.00034124 | P24941 |
| 32 | 32 | 0 | CDK2 | P24941-1 | wp016_TL12.186 | TL12.186 | down | −0.59706382 | 0.00022896 | P24941 |
| 32 | 32 | 0 | CDK2 | P24941-1 | wp080_RSS0680 | RSS0680 | down | −0.42017395 | 0.00011658 | P24941 |
| 32 | 32 | 0 | CDK2 | P24941-1 | wp103_SK.3.91 | SK.3.91 | down | −0.45875316 | 0.00028076 | P24941 |
| 32 | 32 | 0 | CDK2 | P24941-1 | wp104_TMX.01.160.1_0.25 | TMX.01.160.1 | down | −1.02391579 | 2.42E−05 | P24941 |
| 32 | 32 | 0 | CDK2 | P24941-1 | wp104_TMX.01.160.1_1 | TMX.01.160.1 | down | −0.94570816 | 3.61E−05 | P24941 |
| 32 | 32 | 0 | CDK2 | P24941-1 | wp105_SK.3.91 | SK.3.91 | down | −0.45928915 | 1.21E−05 | P24941 |
| 32 | 32 | 0 | CDK2 | P24941-1 | wp107_SK.3.91 | SK.3.91 | down | −0.75834074 | 2.40E−06 | P24941 |
| 32 | 32 | 0 | CDK2 | P24941-1 | wp108_FMF.06.098.1 | FMF.06.098.1 | down | −0.65008429 | 2.88E−05 | P24941 |
| 32 | 32 | 0 | CDK2 | P24941-1 | wp117_RSS0680 | RSS0680 | down | −0.65884683 | 0.00018537 | P24941 |
| 32 | 32 | 0 | CDK2 | P24941-1 | wp131_FMF.06.098.1 | FMF.06.098.1 | down | −0.87250098 | 6.73E−06 | P24941 |
| 32 | 32 | 0 | CDK2 | P24941-1 | wp131_TL12.186 | TL12.186 | down | −0.97996407 | 1.51E−06 | P24941 |
| 32 | 32 | 0 | CDK2 | P24941-1 | wp133_SK.3.91_2 hr | SK.3.91 | down | −0.43057596 | 8.68E−07 | P24941 |
| 32 | 32 | 0 | CDK2 | P24941-1 | wp133_SK.3.91_4 hr | SK.3.91 | down | −0.69080772 | 4.00E−07 | P24941 |
| 32 | 32 | 0 | CDK2 | P24941-1 | wp137_DD.03.106 | DD.03.106 | down | −0.34372555 | 0.00012834 | P24941 |
| 32 | 32 | 0 | CDK2 | P24941-1 | wp137_TL12.186 | TL12.186 | down | −0.91615868 | 6.08E−07 | P24941 |
| 32 | 32 | 0 | CDK2 | P24941-1 | wp137_TL13.97 | TL13.97 | down | −0.78337207 | 1.45E−06 | P24941 |
| 32 | 32 | 0 | CDK2 | P24941-1 | wp146_TMX.01.160 | TMX.01.160 | down | −1.97079006 | 0.00019014 | P24941 |
| 32 | 32 | 0 | CDK2 | P24941-1 | wp146_TMX.02.138 | TMX.02.138 | down | −2.26963739 | 0.00011395 | P24941 |
| 32 | 32 | 0 | CDK2 | P24941-1 | wp146_TMX.02.172 | TMX.02.172 | down | −2.19902827 | 0.0001278 | P24941 |
| 32 | 32 | 0 | CDK2 | P24941-1 | wp146_TMX.02.174 | TMX.02.174 | down | −2.29517392 | 0.00010941 | P24941 |
| 32 | 32 | 0 | CDK2 | P24941-1 | wp147_DD.03.106_2 uM | DD.03.106 | down | −0.49318596 | 9.36E−07 | P24941 |
| 32 | 32 | 0 | CDK2 | P24941-1 | wp147_DD.03.106_1 uM | DD.03.106 | down | −0.32854957 | 7.48E−06 | P24941 |
| 32 | 32 | 0 | CDK2 | P24941-1 | wp147_DD.03.106_4 uM | DD.03.106 | down | −0.52480849 | 6.80E−07 | P24941 |
| 32 | 32 | 0 | CDK2 | P24941-1 | wp160_SK.3.91_2 hr | SK.3.91 | down | −0.38302149 | 4.24E−05 | P24941 |
| 32 | 32 | 0 | CDK2 | P24941-1 | wp160_SK.3.91_4 hr | SK.3.91 | down | −0.64702946 | 5.11E−06 | P24941 |
| 32 | 32 | 0 | CDK2 | P24941-1 | wp172_SK.3.91 | SK.3.91 | down | −0.89959699 | 6.62E−07 | P24941 |
| 30 | 30 | 0 | ABL2 | P42684-5 | wp047_DD.03.119 | DD.03.119 | down | −1.04721702 | 8.46E−06 | P42684 |
| 30 | 30 | 0 | ABL2 | P42684-5 | wp131_FMF.06.098.1 | FMF.06.098.1 | down | −0.64426417 | 3.27E−05 | P42684 |
| 30 | 30 | 0 | ABL2 | P42684-5 | wp131_SB1.G.192.1 | SB1.G.192.1 | down | −2.80008603 | 3.25E−07 | P42684 |
| 30 | 30 | 0 | ABL2 | P42684-5 | wp131_TL12.186 | TL12.186 | down | −0.80230497 | 2.03E−05 | P42684 |
| 30 | 30 | 0 | ABL2 | P42684-5 | wp172_SK.3.91 | SK.3.91 | down | −0.71386032 | 5.86E−05 | P42684 |
| 30 | 30 | 0 | ABL2 | P42684 | wp016_SK.3.91 | SK.3.91 | down | −0.49630383 | 0.00934329 | P42684 |
| 30 | 30 | 0 | ABL2 | P42684 | wp016_SK.3.91_1 uM | SK.3.91 | down | −1.07679169 | 0.00042245 | P42684 |
| 30 | 30 | 0 | ABL2 | P42684 | wp016_SK.3.93 | SK.3.93 | down | −0.52775624 | 0.00743168 | P42684 |
| 30 | 30 | 0 | ABL2 | P42684 | wp045_DB.3.291 | DB.3.291 | down | −0.37389027 | 0.0015394 | P42684 |
| 30 | 30 | 0 | ABL2 | P42684 | wp045_DB0646 | DB0646 | down | −0.5941275 | 0.00022028 | P42684 |
| 30 | 30 | 0 | ABL2 | P42684 | wp045_DB0663 | DB0663 | down | −0.60224285 | 0.00020786 | P42684 |
| 30 | 30 | 0 | ABL2 | P42684 | wp056_SB1.G.187 | SB1.G.187 | down | −0.62521158 | 0.00085332 | P42684 |
| 30 | 30 | 0 | ABL2 | P42684 | wp071_SB1.G.181 | SB1.G.181 | down | −2.40484764 | 2.88E−06 | P42684 |
| 30 | 30 | 0 | ABL2 | P42684 | wp071_SB1.G.192.1 | SB1.G.192.1 | down | −2.44034781 | 2.68E−06 | P42684 |
| 30 | 30 | 0 | ABL2 | P42684 | wp071_SB1.G.200 | SB1.G.200 | down | −2.10312165 | 5.54E−06 | P42684 |
| 30 | 30 | 0 | ABL2 | P42684 | wp107_DB0646 | DB0646 | down | −0.53418096 | 2.63E−06 | P42684 |
| 30 | 30 | 0 | ABL2 | P42684 | wp107_SB1.G.187 | SB1.G.187 | down | −0.64547218 | 5.72E−06 | P42684 |
| 30 | 30 | 0 | ABL2 | P42684 | wp107_SK.3.91 | SK.3.91 | down | −1.03219998 | 6.90E−08 | P42684 |
| 30 | 30 | 0 | ABL2 | P42684 | wp133_SK.3.91_2 hr | SK.3.91 | down | −0.34528942 | 0.00030789 | P42684 |
| 30 | 30 | 0 | ABL2 | P42684 | wp133_SK.3.91_4 hr | SK.3.91 | down | −0.85190156 | 1.47E−06 | P42684 |
| 30 | 30 | 0 | ABL2 | P42684 | wp137_TL12.186 | TL12.186 | down | −0.80296021 | 2.27E−06 | P42684 |
| 30 | 30 | 0 | ABL2 | P42684 | wp137_TL13.97 | TL13.97 | down | −0.5115501 | 2.68E−05 | P42684 |
| 30 | 30 | 0 | ABL2 | P42684 | wp152_DB0646 | DB0646 | down | −0.53620641 | 9.84E−06 | P42684 |
| 30 | 30 | 0 | ABL2 | P42684 | wp152_DB1114 | DB1114 | down | −0.96220387 | 4.01E−07 | P42684 |
| 30 | 30 | 0 | ABL2 | P42684 | wp160_DB0646_4 hr | DB0646 | down | −0.50286107 | 0.00036097 | P42684 |
| 30 | 30 | 0 | ABL2 | P42684 | wp160_DB0646_8 hr | DB0646 | down | −0.72880738 | 8.24E−05 | P42684 |
| 30 | 30 | 0 | ABL2 | P42684 | wp160_SK.3.91_4 hr | SK.3.91 | down | −0.76584714 | 6.75E−05 | P42684 |
| 30 | 30 | 0 | ABL2 | P42684 | wp168_DB0662 | DB0662 | down | −1.20696174 | 8.61E−07 | P42684 |
| 30 | 30 | 0 | ABL2 | P42684 | wp168_DB1113 | DB1113 | down | −1.72300801 | 6.02E−08 | P42684 |
| 30 | 30 | 0 | ABL2 | P42684 | wp195_JWG.148 | JWG.148 | down | −0.51600783 | 3.26E−05 | P42684 |
| 29 | 21 | 8 | ITK | Q08881 | wp016_LT2.49 | LT2.49 | down | −2.21170359 | 1.04E−05 | Q08881 |
| 29 | 21 | 8 | ITK | Q08881 | wp016_SK.3.87 | SK.3.87 | down | −1.86843324 | 2.16E−05 | Q08881 |
| 29 | 21 | 8 | ITK | Q08881 | wp016_SK.3.89 | SK.3.89 | down | −2.04389013 | 1.46E−05 | Q08881 |
| 29 | 21 | 8 | ITK | Q08881 | wp016_SK_3.91 | SK.3.91 | down | −2.30798312 | 8.64E−06 | Q08881 |
| 29 | 21 | 8 | ITK | Q08881 | wp016_SK.3.91_1 uM | SK.3.91 | down | −2.03307674 | 1.50E−05 | Q08881 |
| 29 | 21 | 8 | ITK | Q08881 | wp016_SK.3.93 | SK.3.93 | down | −2.13646794 | 1.21E−05 | Q08881 |
| 29 | 21 | 8 | ITK | Q08881 | wp016_TL12.186 | TL12.186 | down | −2.34241816 | 8.10E−06 | Q08881 |
| 29 | 21 | 8 | ITK | Q08881 | wp103_SK.3.91 | SK.3.91 | down | −1.66311181 | 4.07E−07 | Q08881 |
| 29 | 21 | 8 | ITK | Q08881 | wp107_SK.3.91 | SK.3.91 | down | −1.96261003 | 1.23E−07 | Q08881 |
| 29 | 21 | 8 | ITK | Q08881 | wp108_FMF.06.098.1 | FMF.06.098.1 | down | −0.35570167 | 0.00152252 | Q08881 |
| 29 | 21 | 8 | ITK | Q08881 | wp131_FMF.06.098.1 | FMF.06.098.1 | down | −0.36252401 | 7.26E−05 | Q08881 |
| 29 | 21 | 8 | ITK | Q08881 | wp131_TL12.186 | TL12.186 | down | −2.29194462 | 2.79E−07 | Q08881 |
| 29 | 21 | 8 | ITK | Q08881 | wp133_SK.3.91_1 hr | SK.3.91 | down | −0.37407969 | 3.07E−05 | Q08881 |
| 29 | 21 | 8 | ITK | Q08881 | wp133_SK.3.91_2 hr | SK.3.91 | down | −0.8320613 | 1.18E−07 | Q08881 |
| 29 | 21 | 8 | ITK | Q08881 | wp133_SK.3.91_4 hr | SK.3.91 | down | −1.65362317 | 2.28E−09 | Q08881 |
| 29 | 21 | 8 | ITK | Q08881 | wp134_BSJ.05.037 | BSJ.05.037 | down | −1.81587569 | 2.82E−09 | Q08881 |
| 29 | 21 | 8 | ITK | Q08881 | wp137_TL12.186 | TL12.186 | down | −1.97295153 | 3.11E−09 | Q08881 |
| 29 | 21 | 8 | ITK | Q08881 | wp137_TL13.97 | TL13.97 | down | −2.28045513 | 1.38E−09 | Q08881 |
| 29 | 21 | 8 | ITK | Q08881 | wp160_SK.3.91_2 hr | SK.3.91 | down | −0.79899462 | 2.42E−05 | Q08881 |
| 29 | 21 | 8 | ITK | Q08881 | wp160_SK.3.91_4 hr | SK.3.91 | down | −1.7827166 | 9.43E−07 | Q08881 |
| 29 | 21 | 8 | ITK | Q08881 | wp195_BJG.04.203 | BJG.04.203 | down | −2.43258084 | 1.20E−08 | Q08881 |
| 29 | 29 | 0 | CDK9 | P50750-2 | wp016_LT2.49 | LT2.49 | down | −0.64690989 | 0.00058619 | P50750 |
| 29 | 29 | 0 | CDK9 | P50750-2 | wp016_SK.3.87 | SK.3.87 | down | −0.47143932 | 0.00214728 | P50750 |
| 29 | 29 | 0 | CDK9 | P50750-2 | wp016_SK.3.89 | SK.3.89 | down | −0.47773227 | 0.00203574 | P50750 |
| 29 | 29 | 0 | CDK9 | P50750-2 | wp016_SK.3.91 | SK.3.91 | down | −0.65145933 | 0.00056929 | P50750 |
| 29 | 29 | 0 | CDK9 | P50750-2 | wp016_SK.3.91_1 uM | SK.3.91 | down | −1.35609318 | 2.50E−05 | P50750 |
| 29 | 29 | 0 | CDK9 | P50750-2 | wp016_SK.3.93 | SK.3.93 | down | −0.6376069 | 0.0006227 | P50750 |
| 29 | 29 | 0 | CDK9 | P50750-2 | wp016_TL12.186 | TL12.186 | down | −0.64141174 | 0.00060744 | P50750 |
| 29 | 29 | 0 | CDK9 | P50750-2 | wp068_FMF.04.147.1 | FMF.04.147.1 | down | −0.91419272 | 6.42E−06 | P50750 |
| 29 | 29 | 0 | CDK9 | P50750-2 | wp076_MFH.5.126.1 | MFH.5.126.1 | down | −0.70075617 | 0.00017265 | P50750 |
| 29 | 29 | 0 | CDK9 | P50750-2 | wp103_SK.3.91 | SK.3.91 | down | −0.78566299 | 7.50E−05 | P50750 |
| 29 | 29 | 0 | CDK9 | P50750-2 | wp105_SK.3.91 | SK.3.91 | down | −0.67752013 | 0.0003276 | P50750 |
| 29 | 29 | 0 | CDK9 | P50750-2 | wp107_SK.3.91 | SK.3.91 | down | −1.20018601 | 4.37E−07 | P50750 |
| 29 | 29 | 0 | CDK9 | P50750-2 | wp108_FMF.06.098.1 | FMF.06.098.1 | down | −0.72326367 | 0.00053017 | P50750 |
| 29 | 29 | 0 | CDK9 | P50750-2 | wp131_FMF.06.098.1 | FMF.06.098.1 | down | −0.67906708 | 1.58E−06 | P50750 |
| 29 | 29 | 0 | CDK9 | P50750-2 | wp131_TL12.186 | TL12.186 | down | −1.45133586 | 1.73E−07 | P50750 |
| 29 | 29 | 0 | CDK9 | P50750-2 | wp133_SK.3.91_2 hr | SK.3.91 | down | −0.39705331 | 2.27E−05 | P50750 |
| 29 | 29 | 0 | CDK9 | P50750-2 | wp133_SK.3.91_4 hr | SK.3.91 | down | −1.03652604 | 9.74E−08 | P50750 |
| 29 | 29 | 0 | CDK9 | P50750-2 | wp134_BSJ.04.178 | BSJ.04.178 | down | −1.43008385 | 1.23E−06 | P50750 |
| 29 | 29 | 0 | CDK9 | P50750-2 | wp137_TL12.186 | TL12.186 | down | −1.51079132 | 2.52E−08 | P50750 |
| 29 | 29 | 0 | CDK9 | P50750-2 | wp137_TL13.97 | TL13.97 | down | −1.36977719 | 4.35E−08 | P50750 |
| 29 | 29 | 0 | CDK9 | P50750-2 | wp146_TMX.01.160 | TMX.01.160 | down | −1.18114055 | 0.00017656 | P50750 |
| 29 | 29 | 0 | CDK9 | P50750-2 | wp146_TMX.02.138 | TMX.02.138 | down | −1.88927894 | 3.20E−05 | P50750 |
| 29 | 29 | 0 | CDK9 | P50750-2 | wp146_TMX.02.174 | TMX.02.174 | down | −1.66605266 | 5.06E−05 | P50750 |
| 29 | 29 | 0 | CDK9 | P50750-2 | wp195_BJG.04.203 | BJG.04.203 | down | −0.52251564 | 2.20E−05 | P50750 |
| 29 | 29 | 0 | CDK9 | P50750-2 | wp195_ZZ.01.31B | ZZ.01.31B | down | −0.83695351 | 1.65E−06 | P50750 |
| 29 | 29 | 0 | CDK9 | P50750 | wp160_SK.3.91_2 hr | SK.3.91 | down | −0.40397871 | 0.0009648 | P50750 |
| 29 | 29 | 0 | CDK9 | P50750 | wp160_SK.3.91_4 hr | SK.3.91 | down | −1.06785413 | 2.02E−05 | P50750 |
| 29 | 29 | 0 | CDK9 | P50750 | wp164_SB1.G.194 | SB1.G.194 | down | −0.83914095 | 5.66E−06 | P50750 |
| 29 | 29 | 0 | CDK9 | P50750 | wp172_SK.3.91 | SK.3.91 | down | −1.42346298 | 1.91E−06 | P50750 |
| 21 | 21 | 0 | MAP3K20 | Q9NYL2-2 | wp045_DB0646 | DB0646 | down | −1.18187328 | 0.00020097 | Q9NYL2 |
| 21 | 21 | 0 | MAP3K20 | Q9NYL2-2 | wp046_DB0663 | DB0663 | down | −0.80116168 | 0.00103734 | Q9NYL2 |
| 21 | 21 | 0 | MAP3K20 | Q9NYL2-2 | wp074_dRAF.3 | dRAF.3 | down | −1.85958379 | 0.00013379 | Q9NYL2 |
| 21 | 21 | 0 | MAP3K20 | Q9NYL2-2 | wp081_TL13.178 | TL13.178 | down | −1.40709916 | 7.33E−06 | Q9NYL2 |
| 21 | 21 | 0 | MAP3K20 | Q9NYL2-2 | wp103_DB0646 | DB0646 | down | −0.70040492 | 0.00152783 | Q9NYL2 |
| 21 | 21 | 0 | MAP3K20 | Q9NYL2-2 | wp103_SB1.G.187 | SB1.G.187 | down | −0.62030049 | 0.00254389 | Q9NYL2 |
| 21 | 21 | 0 | MAP3K20 | Q9NYL2-2 | wp107_DB0646 | DB0646 | down | −0.78253999 | 5.84E−05 | Q9NYL2 |
| 21 | 21 | 0 | MAP3K20 | Q9NYL2-2 | wp107_SB1.G.187 | SB1.G.187 | down | −0.43865762 | 0.00109756 | Q9NYL2 |
| 21 | 21 | 0 | MAP3K20 | Q9NYL2-2 | wp114_DB0646 | DB0646 | down | −0.89623063 | 0.000516 | Q9NYL2 |
| 21 | 21 | 0 | MAP3K20 | Q9NYL2-2 | wp114_SB1.G.187 | SB1.G.187 | down | −0.78460442 | 0.0008698 | Q9NYL2 |
| 21 | 21 | 0 | MAP3K20 | Q9NYL2-2 | wp117_DB0646 | DB0646 | down | −0.67008133 | 0.00022491 | Q9NYL2 |
| 21 | 21 | 0 | MAP3K20 | Q9NYL2-2 | wp147_DD.03.106_2 uM | DD.03.106 | down | −0.73882054 | 0.00019406 | Q9NYL2 |
| 21 | 21 | 0 | MAP3K20 | Q9NYL2-2 | wp147_DD.03.106_4 uM | DD.03.106 | down | −0.4901165 | 0.00138958 | Q9NYL2 |
| 21 | 21 | 0 | MAP3K20 | Q9NYL2-2 | wp168_DB0662 | DB0662 | down | −1.00561567 | 0.00069296 | Q9NYL2 |
| 21 | 21 | 0 | MAP3K20 | Q9NYL2-2 | wp168_DB1113 | DB1113 | down | −0.96925895 | 0.00086008 | Q9NYL2 |
| 21 | 21 | 0 | MAP3K20 | Q9NYL2 | wp045_DB0646 | DB0646 | down | −0.42484012 | 0.00440217 | Q9NYL2 |
| 21 | 21 | 0 | MAP3K20 | Q9NYL2 | wp107_DB0646 | DB0646 | down | −0.53002937 | 0.00034757 | Q9NYL2 |
| 21 | 21 | 0 | MAP3K20 | Q9NYL2 | wp147_DD.03.106_2 uM | DD.03.106 | down | −0.40794145 | 2.63E−05 | Q9NYL2 |
| 21 | 21 | 0 | MAP3K20 | Q9NYL2 | wp152_DB0646 | DB0646 | down | −0.65559463 | 3.80E−06 | Q9NYL2 |
| 21 | 21 | 0 | MAP3K20 | Q9NYL2 | wp152_DB1114 | DB1114 | down | −0.75847182 | 1.71E−06 | Q9NYL2 |
| 21 | 21 | 0 | MAP3K20 | Q9NYL2 | wp160_DB0646_2 hr | DB0646 | down | −0.32928749 | 0.00085226 | Q9NYL2 |
| 21 | 21 | 0 | MAP3K20 | Q9NYL2 | wp160_DB0646_4 hr | DB0646 | down | −0.61919345 | 6.98E−05 | Q9NYL2 |
| 21 | 21 | 0 | MAP3K20 | Q9NYL2 | wp160_DB0646_8 hr | DB0646 | down | −0.91969751 | 1.42E−05 | Q9NYL2 |
| 21 | 21 | 0 | MAP3K20 | Q9NYL2 | wp168_DB0662 | DB0662 | down | −0.73704962 | 4.97E−07 | Q9NYL2 |
| 21 | 21 | 0 | MAP3K20 | Q9NYL2 | wp168_DB1113 | DB1113 | down | −1.38002213 | 9.21E−09 | Q9NYL2 |
| 21 | 21 | 0 | MAP3K20 | Q9NYL2 | wp172_DB0646 | DB0646 | down | −0.38636821 | 0.00116243 | Q9NYL2 |
| 26 | 26 | 0 | ULK1 | O75385 | wp045_DB0614 | DB0614 | down | −0.86904479 | 0.00130447 | O75385 |
| 26 | 26 | 0 | ULK1 | O75385 | wp045_RSS0628 | RSS0628 | down | −0.55828658 | 0.00754393 | O75385 |
| 26 | 26 | 0 | ULK1 | O75385 | wp080_RSS0680 | RSS0680 | down | −0.40565494 | 0.00077443 | O75385 |
| 26 | 26 | 0 | ULK1 | O75385 | wp103_SK.3.91 | SK.3.91 | down | −1.33242832 | 2.42E−05 | O75385 |
| 26 | 26 | 0 | ULK1 | O75385 | wp105_SK.3.91 | SK.3.91 | down | −1.71181527 | 2.03E−06 | O75385 |
| 26 | 26 | 0 | ULK1 | O75385 | wp107_SK.3.91 | SK.3.91 | down | −1.16217391 | 2.25E−06 | O75385 |
| 26 | 26 | 0 | ULK1 | O75385 | wp108_FMF.06.098.1 | FMF.06.098.1 | down | −0.68797245 | 3.40E−05 | O75385 |
| 26 | 26 | 0 | ULK1 | O75385 | wp117_DB0614 | DB0614 | down | −1.10523852 | 3.49E−07 | O75385 |
| 26 | 26 | 0 | ULK1 | O75385 | wp117_RSS0680 | RSS0680 | down | −0.49956121 | 2.12E−05 | O75385 |
| 26 | 26 | 0 | ULK1 | O75385 | wp131_FMF.06.098.1 | FMF.06.098.1 | down | −0.96289442 | 7.54E−07 | O75385 |
| 26 | 26 | 0 | ULK1 | O75385 | wp131_TL12.186 | TL12.186 | down | −3.01039002 | 1.35E−07 | O75385 |
| 26 | 26 | 0 | ULK1 | O75385 | wp133_SK.3.91_1 hr | SK.3.91 | down | −1.47214609 | 2.08E−07 | O75385 |
| 26 | 26 | 0 | ULK1 | O75385 | wp133_SK.3.91_2 hr | SK.3.91 | down | −2.77856814 | 1.53E−09 | O75385 |
| 26 | 26 | 0 | ULK1 | O75385 | wp133_SK.3.91_4 hr | SK.3.91 | down | −3.57223887 | 3.46E−10 | O75385 |
| 26 | 26 | 0 | ULK1 | O75385 | wp134_BSJ.04.178 | BSJ.04.178 | down | −1.68293601 | 6.45E−07 | O75385 |
| 26 | 26 | 0 | ULK1 | O75385 | wp137_TL12.186 | TL12.186 | down | −2.23700808 | 3.64E−08 | O75385 |
| 26 | 26 | 0 | ULK1 | O75385 | wp137_TL13.97 | TL13.97 | down | −1.87718947 | 9.68E−08 | O75385 |
| 26 | 26 | 0 | ULK1 | O75385 | wp152_DB1114 | DB1114 | down | −0.32490956 | 0.00234879 | O75385 |
| 26 | 26 | 0 | ULK1 | O75385 | wp160_DB0646_4 hr | DB0646 | down | −0.34124989 | 0.00822188 | O75385 |
| 26 | 26 | 0 | ULK1 | O75385 | wp160_DB0646_8 hr | DB0646 | down | −0.39893919 | 0.00467464 | O75385 |
| 26 | 26 | 0 | ULK1 | O75385 | wp160_SK.3.91_1 hr | SK.3.91 | down | −1.46578059 | 2.87E−05 | O75385 |
| 26 | 26 | 0 | ULK1 | O75385 | wp160_SK.3.91_2 hr | SK.3.91 | down | −2.58299222 | 2.90E−06 | O75385 |
| 26 | 26 | 0 | ULK1 | O75385 | wp160_SK.3.91_4 hr | SK.3.91 | down | −3.08472872 | 1.41E−06 | O75385 |
| 26 | 26 | 0 | ULK1 | O75385 | wp168_DB0662 | DB0662 | down | −0.35819696 | 0.00089108 | O75385 |
| 26 | 26 | 0 | ULK1 | O75385 | wp168_DB1113 | DB1113 | down | −0.35200582 | 0.00052121 | O75385 |
| 26 | 26 | 0 | ULK1 | O75385 | wp195_BSJ.05.026 | BSJ.05.026 | down | −0.44235632 | 0.00010145 | O75385 |
| 25 | 6 | 19 | CLK1 | P49759-3 | wp068_FMF.04.147.1 | FMF.04.147.1 | down | −0.64616419 | 8.28E−05 | P49759 |
| 25 | 6 | 19 | CLK1 | P49759-3 | wp076_MFH.5.126.1 | MFH.5.126.1 | down | −1.49375117 | 0.00890458 | P49759 |
| 25 | 6 | 19 | CLK1 | P49759-3 | wp146_TMX.02.138 | TMX.02.138 | down | −0.46477099 | 0.00955404 | P49759 |
| 25 | 6 | 19 | CLK1 | P49759-3 | wp146_TMX.02.174 | TMX.02.174 | down | −0.46058638 | 0.00984095 | P49759 |
| 25 | 6 | 19 | CLK1 | P49759 | wp168_DB0661 | DB0661 | down | −0.3318662 | 0.00226003 | P49759 |
| 25 | 6 | 19 | CLK1 | P49759 | wp195_ZZ.01.31B | ZZ.01.31B | down | −0.34824746 | 0.00043294 | P49759 |
| 24 | 24 | 0 | CDK7 | P50613 | wp016_LT2.49 | LT2.49 | down | −1.01369971 | 0.00050766 | P50613 |
| 24 | 24 | 0 | CDK7 | P50613 | wp016_SK.3.87 | SK.3.87 | down | −0.53251198 | 0.00674966 | P50613 |
| 24 | 24 | 0 | CDK7 | P50613 | wp016_SK.3.89 | SK.3.89 | down | −0.79593346 | 0.00138086 | P50613 |
| 24 | 24 | 0 | CDK7 | P50613 | wp016_SK.3.91 | SK.3.91 | down | −0.52362346 | 0.00719248 | P50613 |
| 24 | 24 | 0 | CDK7 | P50613 | wp016_SK.3.93 | SK.3.93 | down | −0.80568087 | 0.00131388 | P50613 |
| 24 | 24 | 0 | CDK7 | P50613 | wp016_TL12.186 | TL12.186 | down | −0.76033079 | 0.0016635 | P50613 |
| 24 | 24 | 0 | CDK7 | P50613 | wp076_MFH.5.103.1 | MFH.5.103.1 | down | −0.5097881 | 0.00867353 | P50613 |
| 24 | 24 | 0 | CDK7 | P50613 | wp080_RSS0680 | RSS0680 | down | −1.01653909 | 1.36E−05 | P50613 |
| 24 | 24 | 0 | CDK7 | P50613 | wp104_TMX.01.160.1_0.25 | TMX.01.160.1 | down | −0.49410632 | 3.25E−05 | P50613 |
| 24 | 24 | 0 | CDK7 | P50613 | wp104_TMX.01.160.1_1 | TMX.01.160.1 | down | −0.52036265 | 2.51E−05 | P50613 |
| 24 | 24 | 0 | CDK7 | P50613 | wp105_SK.3.91 | SK.3.91 | down | −0.63070377 | 0.00014583 | P50613 |
| 24 | 24 | 0 | CDK7 | P50613 | wp107_SK.3.91 | SK.3.91 | down | −0.46317118 | 0.00015718 | P50613 |
| 24 | 24 | 0 | CDK7 | P50613 | wp108_FMF.06.098.1 | FMF.06.098.1 | down | −0.901606 | 4.46E−05 | P50613 |
| 24 | 24 | 0 | CDK7 | P50613 | wp114_FMF.06.098.1 | FMF.06.098.1 | down | −1.13691023 | 7.49E−05 | P50613 |
| 24 | 24 | 0 | CDK7 | P50613 | wp114_SK.3.91 | SK.3.91 | down | −0.52554456 | 0.00158683 | P50613 |
| 24 | 24 | 0 | CDK7 | P50613 | wp117_RSS0680 | RSS0680 | down | −1.21942395 | 1.09E−06 | P50613 |
| 24 | 24 | 0 | CDK7 | P50613 | wp131_FMF.06.098.1 | FMF.06.098.1 | down | −0.97563943 | 4.06E−06 | P50613 |
| 24 | 24 | 0 | CDK7 | P50613 | wp131_TL12.186 | TL12.186 | down | −0.64465696 | 5.04E−06 | P50613 |
| 24 | 24 | 0 | CDK7 | P50613 | wp137_TL12.186 | TL12.186 | down | −0.74375581 | 4.69E−06 | P50613 |
| 24 | 24 | 0 | CDK7 | P50613 | wp137_TL13.97 | TL13.97 | down | −1.04974514 | 6.99E−07 | P50613 |
| 24 | 24 | 0 | CDK7 | P50613 | wp146_TMX.01.160 | TMX.01.160 | down | −0.60101722 | 0.00259946 | P50613 |
| 24 | 24 | 0 | CDK7 | P50613 | wp146_TMX.02.138 | TMX.02.138 | down | −0.52389488 | 0.00417221 | P50613 |
| 24 | 24 | 0 | CDK7 | P50613 | wp146_TMX.02.174 | TMX.02.174 | down | −0.58395101 | 0.00287249 | P50613 |
| 24 | 24 | 0 | CDK7 | P50613 | wp172_SK.3.91 | SK.3.91 | down | −1.18621501 | 1.42E−06 | P50613 |
| 24 | 24 | 0 | MAPK9 | P45984-1 | wp045_DB0614 | DB0614 | down | −0.52444514 | 9.43E−05 | P45984 |
| 24 | 24 | 0 | MAPK9 | P45984-1 | wp045_DB0646 | DB0646 | down | −1.34381144 | 1.55E−06 | P45984 |
| 24 | 24 | 0 | MAPK9 | P45984-1 | wp045_DB0663 | DB0663 | down | −0.59800878 | 5.34E−05 | P45984 |
| 24 | 24 | 0 | MAPK9 | P45984-1 | wp103_DB0646 | DB0646 | down | −1.04527438 | 6.88E−06 | P45984 |
| 24 | 24 | 0 | MAPK9 | P45984-1 | wp104_TMX.01.160.1_0.25 | TMX.01.160.1 | down | −1.6350769 | 5.99E−07 | P45984 |
| 24 | 24 | 0 | MAPK9 | P45984-1 | wp104_TMX.01.160.1_1 | TMX.01.160.1 | down | −1.49641477 | 9.43E−07 | P45984 |
| 24 | 24 | 0 | MAPK9 | P45984-1 | wp105_DB0646 | DB0646 | down | −0.92904564 | 4.34E−07 | P45984 |
| 24 | 24 | 0 | MAPK9 | P45984-1 | wp107_DB0646 | DB0646 | down | −1.41416868 | 3.84E−07 | P45984 |
| 24 | 24 | 0 | MAPK9 | P45984-1 | wp114_DB0646 | DB0646 | down | −0.5077738 | 0.00084333 | P45984 |
| 24 | 24 | 0 | MAPK9 | P45984-1 | wp117_DB0614 | DB0614 | down | −0.56396401 | 3.25E−06 | P45984 |
| 24 | 24 | 0 | MAPK9 | P45984-1 | wp117_DB0646 | DB0646 | down | −1.48767754 | 3.49E−08 | P45984 |
| 24 | 24 | 0 | MAPK9 | P45984-1 | wp146_TMX.01.160 | TMX.01.160 | down | −3.3199286 | 1.41E−06 | P45984 |
| 24 | 24 | 0 | MAPK9 | P45984-1 | wp146_TMX.02.138 | TMX.02.138 | down | −1.51840501 | 2.48E−05 | P45984 |
| 24 | 24 | 0 | MAPK9 | P45984-1 | wp146_TMX.02.172 | TMX.02.172 | down | −1.09136777 | 8.25E−05 | P45984 |
| 24 | 24 | 0 | MAPK9 | P45984-1 | wp146_TMX.02.174 | TMX.02.174 | down | −1.03106004 | 0.00010144 | P45984 |
| 24 | 24 | 0 | MAPK9 | P45984-1 | wp160_DB0646_2 hr | DB0646 | down | −0.75553147 | 0.00059819 | P45984 |
| 24 | 24 | 0 | MAPK9 | P45984-1 | wp160_DB0646_4 hr | DB0646 | down | −1.49807566 | 3.92E−05 | P45984 |
| 24 | 24 | 0 | MAPK9 | P45984-1 | wp160_DB0646_8 hr | DB0646 | down | −2.13724247 | 9.35E−06 | P45984 |
| 24 | 24 | 0 | MAPK9 | P45984-1 | wp168_DB0661 | DB0661 | down | −1.96149119 | 1.90E−05 | P45984 |
| 24 | 24 | 0 | MAPK9 | P45984-1 | wp168_DB0662 | DB0662 | down | −2.61096771 | 3.80E−06 | P45984 |
| 24 | 24 | 0 | MAPK9 | P45984-1 | wp168_DB1113 | DB1113 | down | −1.21591726 | 1.54E−05 | P45984 |
| 24 | 24 | 0 | MAPK9 | P45984-1 | wp172_DB0646 | DB0646 | down | −0.83426727 | 1.34E−05 | P45984 |
| 24 | 24 | 0 | MAPK9 | P45984 | wp152_DB0646 | DB0646 | down | −2.16078368 | 1.65E−08 | P45984 |
| 24 | 24 | 0 | MAPK9 | P45984 | wp152_DB1114 | DB1114 | down | −1.18105335 | 4.63E−07 | P45984 |
| 22 | 22 | 0 | STK17A | Q9UEE5 | wp016_SK.3.91_1 uM | SK.3.91 | down | −0.70324316 | 0.00247704 | Q9UEE5 |
| 22 | 22 | 0 | STK17A | Q9UEE5 | wp045_DB0614 | DB0614 | down | −1.12812084 | 0.00048777 | Q9UEE5 |
| 22 | 22 | 0 | STK17A | Q9UEE5 | wp045_RSS0628 | RSS0628 | down | −1.47977008 | 0.0001535 | Q9UEE5 |
| 22 | 22 | 0 | STK17A | Q9UEE5 | wp076_MFH.5.115.2 | MFH.5.115.2 | down | −0.627714 | 0.00312898 | Q9UEE5 |
| 22 | 22 | 0 | STK17A | Q9UEE5 | wp076_MFH.5.126.1 | MFH.5.126.1 | down | −0.89837899 | 0.00071646 | Q9UEE5 |
| 22 | 22 | 0 | STK17A | Q9UEE5 | wp080_RSS0680 | RSS0680 | down | −1.11830963 | 2.69E−05 | Q9UEE5 |
| 22 | 22 | 0 | STK17A | QQUEE5 | wp088_SB1.G.194 | SB1.G.194 | down | −0.64337232 | 0.00518458 | Q9UEE5 |
| 22 | 22 | 0 | STK17A | Q9UEE5 | wp103_SK.3.91 | SK.3.91 | down | −0.62008614 | 0.0007121 | Q9UEE5 |
| 22 | 22 | 0 | STK17A | Q9UEE5 | wp107_SK.3.91 | SK.3.91 | down | −0.69842105 | 2.92E−05 | Q9UEE5 |
| 22 | 22 | 0 | STK17A | Q9UEE5 | wp117_DB0614 | DB0614 | down | −0.96903957 | 6.04E−06 | Q9UEE5 |
| 22 | 22 | 0 | STK17A | Q9UEE5 | wp117_RSS0680 | RSS0680 | down | −1.37850603 | 6.54E−07 | Q9UEE5 |
| 22 | 22 | 0 | STK17A | Q9UEE5 | wp118_INY.03.041.01 | INY.03.041.01 | down | −0.43611965 | 0.00056789 | Q9UEE5 |
| 22 | 22 | 0 | STK17A | Q9UEE5 | wp133_SK.3.91_4 hr | SK.3.91 | down | −0.54579227 | 5.77E−06 | Q9UEE5 |
| 22 | 22 | 0 | STK17A | Q9UEE5 | wp134_BS.J.04.178 | BSJ.04.178 | down | −0.71068475 | 0.00015935 | Q9UEE5 |
| 22 | 22 | 0 | STK17A | Q9UEE5 | wp134_DB118430 | DB118430 | down | −0.34974668 | 0.00584854 | Q9UEE5 |
| 22 | 22 | 0 | STK17A | Q9UEE5 | wp135_INY.03.041_01_8 hr | INY.03.041.01 | down | −0.49703192 | 5.90E−06 | Q9UEE5 |
| 22 | 22 | 0 | STK17A | Q9UEE5 | wp137_TL12.186 | TL12.186 | down | −0.50543286 | 0.00022635 | Q9UEE5 |
| 22 | 22 | 0 | STK17A | Q9UEE5 | wp149_ZNL.03.127 | ZNL.03.127 | down | −0.46045245 | 0.00131931 | Q9UEE5 |
| 22 | 22 | 0 | STK17A | Q9UEE5 | wp172_SK.3.91 | SK.3.91 | down | −0.39632415 | 0.00091213 | Q9UEE5 |
| 22 | 22 | 0 | STK17A | QQUEE5 | wp195_BSJ.05.026 | BSJ.05.026 | down | −0.52085379 | 7.52E−05 | Q9UEE5 |
| 22 | 22 | 0 | STK17A | Q9UEE5 | wp195_INY.04.125.01 | INY.04.125.01 | down | −0.40838196 | 0.00027251 | Q9UEE5 |
| 22 | 22 | 0 | STK17A | Q9UEE5 | wp195_JWG.123 | JWG.123 | down | −0.64506069 | 2.37E−05 | Q9UEE5 |
| 22 | 21 | 1 | AURKB | Q96GD4-5 | wp080_dAURK.4 | dAURK.4 | down | −0.39317896 | 0.00126989 | Q96GD4 |
| 22 | 21 | 1 | AURKB | Q96GD4-5 | wp088_SB1.G.194 | SB1.G.194 | down | −0.3846079 | 0.00043795 | Q96GD4 |
| 22 | 21 | 1 | AURKB | Q96GD4-5 | wp103_SK.3.91 | SK.3.91 | down | −0.39660477 | 0.00199498 | Q96GD4 |
| 22 | 21 | 1 | AURKB | Q96GD4-5 | wp105_SK.3.91 | SK.3.91 | down | −0.70039723 | 1.17E−05 | Q96GD4 |
| 22 | 21 | 1 | AURKB | Q96GD4-5 | wp107_SK.3.91 | SK.3.91 | down | −0.44298608 | 0.00043277 | Q96GD4 |
| 22 | 21 | 1 | AURKB | Q96GD4-5 | wp108_FMF.06.098.1 | FMF.06.098.1 | down | −0.52457519 | 0.00079222 | Q96GD4 |
| 22 | 21 | 1 | AURKB | Q96GD4-5 | wp131_FMF.06.098.1 | FMF.06.098.1 | down | −0.67258837 | 1.62E−06 | Q96GD4 |
| 22 | 21 | 1 | AURKB | Q96GD4-5 | wp131_TL12.186 | TL12.186 | down | −0.99377961 | 4.19E−07 | Q96GD4 |
| 22 | 21 | 1 | AURKB | Q96GD4-5 | wp133_SK.3.91_1 hr | SK.3.91 | down | −0.4923076 | 2.39E−05 | Q96GD4 |
| 22 | 21 | 1 | AURKB | Q96GD4-5 | wp133_SK.3.91_2 hr | SK.3.91 | down | −0.53326873 | 1.90E−05 | Q96GD4 |
| 22 | 21 | 1 | AURKB | Q96GD4-5 | wp133_SK.3.91_4 hr | SK.3.91 | down | −0.80071722 | 1.16E−06 | Q96GD4 |
| 22 | 21 | 1 | AURKB | Q96GD4-5 | wp134_BSJ.04.178 | BSJ.04.178 | down | −0.5073742 | 5.55E−05 | Q96GD4 |
| 22 | 21 | 1 | AURKB | Q96GD4-5 | wp137_TL12.186 | TL12.186 | down | −0.97113527 | 5.79E−06 | Q96GD4 |
| 22 | 21 | 1 | AURKB | Q96GD4-5 | wp137_TL13.97 | TL13.97 | down | −0.7522987 | 2.34E−05 | Q96GD4 |
| 22 | 21 | 1 | AURKB | Q96GD4-5 | wp146_TMX.02.138 | TMX.02.138 | down | −1.83198408 | 5.46E−05 | Q96GD4 |
| 22 | 21 | 1 | AURKB | Q96GD4-5 | wp146_TMX.02.172 | TMX.02.172 | down | −0.48560093 | 0.00611225 | Q96GD4 |
| 22 | 21 | 1 | AURKB | Q96GD4-5 | wp146_TMX.02.174 | TMX.02.174 | down | −1.57784438 | 9.40E−05 | Q96GD4 |
| 22 | 21 | 1 | AURKB | Q96GD4 | wp160_SK.3.91_4 hr | SK.3.91 | down | −0.49836765 | 0.00029365 | Q96GD4 |
| 22 | 21 | 1 | AURKB | Q96GD4 | wp164_SB1.G.194 | SB1.G.194 | down | −0.48829359 | 0.00015297 | Q96GD4 |
| 22 | 21 | 1 | AURKB | Q96GD4 | wp172_SK.3.91 | SK.3.91 | down | −1.10992709 | 1.05E−06 | Q96GD4 |
| 22 | 21 | 1 | AURKB | Q96GD4 | wp195_BJG.04.203 | BJG.04.203 | down | −0.50638583 | 5.34E−05 | Q96GD4 |
| 22 | 13 | 9 | PDK1 | Q15118 | wp056_SB1.G.187 | SB1.G.187 | down | −0.41336033 | 0.00581941 | Q15118 |
| 22 | 13 | 9 | PDK1 | Q15118 | wp056_ZNL.02.096 | ZNL.02.096 | down | −0.36853617 | 0.00885333 | Q15118 |
| 22 | 13 | 9 | PDK1 | Q15118 | wp081_INY.01.140.1 | INY.01.140.1 | down | −0.43283814 | 0.00045347 | Q15118 |
| 22 | 13 | 9 | PDK1 | Q15118 | wp081_SB1.G.192.1 | SB1.G.192.1 | down | −0.3387968 | 0.00153499 | Q15118 |
| 22 | 13 | 9 | PDK1 | Q15118 | wp084_BSJ.03.123 | BSJ.03.123 | down | −0.56657829 | 0.001314 | Q15118 |
| 22 | 13 | 9 | PDK1 | Q15118 | wp084_BS.J.03.204 | BSJ.03.204 | down | −0.37669445 | 0.00664955 | Q15118 |
| 22 | 13 | 9 | PDK1 | Q15118 | wp084_BSJ.04.132 | BSJ.04.132 | down | −0.75690792 | 5.17E−05 | Q15118 |
| 22 | 13 | 9 | PDK1 | Q15118 | wp137_INY.02.164 | INY.02.164 | down | −0.44191096 | 5.25E−05 | Q15118 |
| 22 | 13 | 9 | PDK1 | Q15118 | wp147_DD.03.106_1 uM | DD.03.106 | down | −0.33628597 | 0.00477792 | Q15118 |
| 22 | 13 | 9 | PDK1 | Q15118 | wp147_DD.03.106_4 uM | DD.03.106 | down | −0.65916892 | 0.00020603 | Q15118 |
| 22 | 13 | 9 | PDK1 | Q15118 | wp147_DD.03.107_1 uM | DD.03.107 | down | −0.47218049 | 0.00102755 | Q15118 |
| 22 | 13 | 9 | PDK1 | Q15118 | wp147_DD.03.107_2 uM | DD.03.107 | down | −0.45342088 | 0.0012423 | Q15118 |
| 22 | 13 | 9 | PDK1 | Q15118 | wp147_DD.03.107_4 uM | DD.03.107 | down | −1.10818756 | 1.53E−05 | Q15118 |
| 22 | 8 | 14 | PLK1 | P53350 | wp076_MFH.5.115.2 | MFH.5.115.2 | down | −0.56002665 | 0.00023041 | P53350 |
| 22 | 8 | 14 | PLK1 | P53350 | wp076_MFH.5.126.1 | MFH.5.126.1 | down | −1.07094582 | 1.37E−05 | P53350 |
| 22 | 8 | 14 | PLK1 | P53350 | wp133_SK.3.91_1 hr | SK.3.91 | down | −0.55914584 | 4.97E−06 | P53350 |
| 22 | 8 | 14 | PLK1 | P53350 | wp133_SK.3.91_2 hr | SK.3.91 | down | −0.58042437 | 3.54E−06 | P53350 |
| 22 | 8 | 14 | PLK1 | P53350 | wp133_SK.3.91_4 hr | SK.3.91 | down | −0.74818833 | 4.64E−07 | P53350 |
| 22 | 8 | 14 | PLK1 | P53350 | wp147_DD.03.106_4 uM | DD.03.106 | down | −0.35331461 | 0.00032652 | P53350 |
| 22 | 8 | 14 | PLK1 | P53350 | wp152_DB0646 | DB0646 | down | −0.33103397 | 1.50E−05 | P53350 |
| 22 | 8 | 14 | PLK1 | P53350 | wp168_DB0661 | DB0661 | down | −0.34532948 | 0.00046288 | P53350 |
| 20 | 20 | 0 | TNK2 | Q07912-3 | wp045_DB0663 | DB0663 | down | −0.92707094 | 0.00873098 | Q07912 |
| 20 | 20 | 0 | TNK2 | Q07912-3 | wp071_SB1.G.192.1 | SB1.G.192.1 | down | −1.72783854 | 0.00322608 | Q07912 |
| 20 | 20 | 0 | TNK2 | Q07912-3 | wp081_SB1.G.192.1 | SB1.G.192.1 | down | −0.90236445 | 0.00031667 | Q07912 |
| 20 | 20 | 0 | TNK2 | Q07912-3 | wp107_DB0646 | DB0646 | down | −0.82583788 | 0.00522429 | Q07912 |
| 20 | 20 | 0 | TNK2 | Q07912-3 | wp107_SK.3.91 | SK.3.91 | down | −1.17998954 | 0.00102344 | Q07912 |
| 20 | 20 | 0 | TNK2 | Q07912-3 | wp117_DB0646 | DB0646 | down | −0.98333049 | 1.69E−05 | Q07912 |
| 20 | 20 | 0 | TNK2 | Q07912-3 | wp149_ZNL.03.127 | ZNL.03.127 | down | −0.87776404 | 0.00039242 | Q07912 |
| 20 | 20 | 0 | TNK2 | Q07912-1 | wp108_FMF.06.098.1 | FMF.06.098.1 | down | −0.51083181 | 0.00101884 | Q07912 |
| 20 | 20 | 0 | TNK2 | Q07912-1 | wp131_FMF.06.098.1 | FMF.06.098.1 | down | −0.58047902 | 0.0001129 | Q07912 |
| 20 | 20 | 0 | TNK2 | Q07912-1 | wp131_SB1.G.192.1 | SB1.G.192.1 | down | −1.48815065 | 9.34E−07 | Q07912 |
| 20 | 20 | 0 | TNK2 | Q07912-1 | wp131_TL12.186 | TL12.186 | down | −2.25071196 | 8.33E−08 | Q07912 |
| 20 | 20 | 0 | TNK2 | Q07912-1 | wp133_SK.3.91_2 hr | SK.3.91 | down | −1.05332668 | 2.49E−05 | Q07912 |
| 20 | 20 | 0 | TNK2 | Q07912-1 | wp133_SK.3.91_4 hr | SK.3.91 | down | −2.26072154 | 3.94E−07 | Q07912 |
| 20 | 20 | 0 | TNK2 | Q07912-1 | wp134_DB118430 | DB118430 | down | −0.57499273 | 0.00046164 | Q07912 |
| 20 | 20 | 0 | TNK2 | Q07912-1 | wp137_TL12.186 | TL12.186 | down | −1.57528356 | 1.65E−05 | Q07912 |
| 20 | 20 | 0 | TNK2 | Q07912-1 | wp137_TL13.97 | TL13.97 | down | −1.97099067 | 4.83E−06 | Q07912 |
| 20 | 20 | 0 | TNK2 | Q07912-1 | wp168_DB0661 | DB0661 | down | −0.3873102 | 0.00973703 | Q07912 |
| 20 | 20 | 0 | TNK2 | Q07912-1 | wp168_DB0662 | DB0662 | down | −0.69426781 | 0.0010628 | Q07912 |
| 20 | 20 | 0 | TNK2 | Q07912-1 | wp168_DB1113 | DB1113 | down | −0.53509098 | 0.00343997 | Q07912 |
| 20 | 20 | 0 | TNK2 | Q07912 | wp152_DB1114 | DB1114 | down | −0.59780861 | 0.00232807 | Q07912 |
| 20 | 20 | 0 | MAPK8 | P45983-4 | wp045_DB0614 | DB0614 | down | −0.68821538 | 2.69E−05 | P45983 |
| 20 | 20 | 0 | MAPK8 | P45983-4 | wp045_DB0646 | DB0646 | down | −0.97741074 | 5.81E−06 | P45983 |
| 20 | 20 | 0 | MAPK8 | P45983-4 | wp104_TMX.01.160.1_0.25 | TMX.01.160.1 | down | −0.33383842 | 0.00225052 | P45983 |
| 20 | 20 | 0 | MAPK8 | P45983-4 | wp104_TMX.01.160.1_1 | TMX.01.160.1 | down | −0.47600404 | 0.00042775 | P45983 |
| 20 | 20 | 0 | MAPK8 | P45983-4 | wp146_TMX.01.160 | TMX.01.160 | down | −2.11240658 | 3.16E−05 | P45983 |
| 20 | 20 | 0 | MAPK8 | P45983-4 | wp146_TMX.02.138 | TMX.02.138 | down | −0.98125861 | 0.00050969 | P45983 |
| 20 | 20 | 0 | MAPK8 | P45983-4 | wp146_TMX.02.172 | TMX.02.172 | down | −0.55241107 | 0.00385196 | P45983 |
| 20 | 20 | 0 | MAPK8 | P45983-4 | wp146_TMX.02.174 | TMX.02.174 | down | −0.77613827 | 0.00117603 | P45983 |
| 20 | 20 | 0 | MAPK8 | P45983-4 | wp168_DB0661 | DB0661 | down | −0.5033493 | 6.27E−06 | P45983 |
| 20 | 20 | 0 | MAPK8 | P45983-4 | wp168_DB0662 | DB0662 | down | −0.89606777 | 4.14E−08 | P45983 |
| 20 | 20 | 0 | MAPK8 | P45983-4 | wp168_DB1113 | DB1113 | down | −0.36415646 | 6.70E−06 | P45983 |
| 20 | 20 | 0 | MAPK8 | P45983-3 | wp105_DB0646 | DB0646 | down | −0.40939353 | 0.00168302 | P45983 |
| 20 | 20 | 0 | MAPK8 | P45983-3 | wp160_DB0646_4 hr | DB0646 | down | −0.61095606 | 1.34E−05 | P45983 |
| 20 | 20 | 0 | MAPK8 | P45983-3 | wp160_DB0646_8 hr | DB0646 | down | −1.01015919 | 1.76E−06 | P45983 |
| 20 | 20 | 0 | MAPK8 | P45983-2 | wp103_DB0646 | DB0646 | down | −0.61087482 | 4.72E−05 | P45983 |
| 20 | 20 | 0 | MAPK8 | P45983-2 | wp107_DB0646 | DB0646 | down | −0.72301701 | 1.57E−06 | P45983 |
| 20 | 20 | 0 | MAPK8 | P45983-2 | wp117_DB0614 | DB0614 | down | −0.63283074 | 5.89E−06 | P45983 |
| 20 | 20 | 0 | MAPK8 | P45983-2 | wp117_DB0646 | DB0646 | down | −0.78345847 | 1.13E−06 | P45983 |
| 20 | 20 | 0 | MAPK8 | P45983-1 | wp172_DB0646 | DB0646 | down | −0.37601203 | 0.0007319 | P45983 |
| 20 | 20 | 0 | MAPK8 | P45983 | wp152_DB0646 | DB0646 | down | −0.84329995 | 5.91E−07 | P45983 |
| 20 | 18 | 2 | STK17B | O94768 | wp016_SK.3.91_1 uM | SK.3.91 | down | −0.32695118 | 0.00453516 | O94768 |
| 20 | 18 | 2 | STK17B | O94768 | wp017_WH.9533.153 | WH.9533.153 | down | −0.75946161 | 9.61E−05 | O94768 |
| 20 | 18 | 2 | STK17B | O94768 | wp045_DB0614 | DB0614 | down | −0.98286262 | 0.00028595 | O94768 |
| 20 | 18 | 2 | STK17B | O94768 | wp045_RSS0628 | RSS0628 | down | −1.07480382 | 0.00019516 | O94768 |
| 20 | 18 | 2 | STK17B | O94768 | wp056_WH.10417.099 | WH.10417.099 | down | −1.50721695 | 5.81E−06 | O94768 |
| 20 | 18 | 2 | STK17B | O94768 | wp076_MFH.5.126.1 | MFH.5.126.1 | down | −0.58207492 | 0.00070772 | O94768 |
| 20 | 18 | 2 | STK17B | O94768 | wp080_RSS0680 | RSS0680 | down | −1.37460103 | 2.33E−06 | O94768 |
| 20 | 18 | 2 | STK17B | O94768 | wp103_WH.10417.099 | WH.10417.099 | down | −1.29227486 | 0.00012486 | O94768 |
| 20 | 18 | 2 | STK17B | O94768 | wp105_WH.10417.099 | WH.10417.099 | down | −0.79237629 | 0.00030139 | O94768 |
| 20 | 18 | 2 | STK17B | O94768 | wp107_WH.10417.099 | WH.10417.099 | down | −1.47217329 | 8.44E−07 | O94768 |
| 20 | 18 | 2 | STK17B | O94768 | wp117_DB0614 | DB0614 | down | −0.77706518 | 1.34E−05 | O94768 |
| 20 | 18 | 2 | STK17B | O94768 | wp117_RSS0680 | RSS0680 | down | −1.37326618 | 3.58E−07 | O94768 |
| 20 | 18 | 2 | STK17B | O94768 | wp133_SK.3.91_4 hr | SK.3.91 | down | −0.54501079 | 8.65E−06 | O94768 |
| 20 | 18 | 2 | STK17B | O94768 | wp134_BSJ.04.178 | BSJ.04.178 | down | −1.60440039 | 9.69E−07 | O94768 |
| 20 | 18 | 2 | STK17B | O94768 | wp172_WH.10417.099 | WH.10417.099 | down | −1.23426737 | 3.84E−05 | O94768 |
| 20 | 18 | 2 | STK17B | O94768 | wp195_BJG.04.203 | BJG.04.203 | down | −0.5951327 | 4.00E−05 | O94768 |
| 20 | 18 | 2 | STK17B | O94768 | wp195_BSJ.05.026 | BSJ.05.026 | down | −0.92992525 | 3.48E−06 | O94768 |
| 20 | 18 | 2 | STK17B | O94768 | wp195_JWG.123 | JWG.123 | down | −1.87165064 | 7.13E−08 | O94768 |
| 20 | 13 | 7 | BUB1 | O43683 | wp045_DB0646 | DB0646 | down | −0.33782295 | 0.00040438 | O43683 |
| 20 | 13 | 7 | BUB1 | O43683 | wp076_MFH.5.115.2 | MFH.5.115.2 | down | −0.51592084 | 8.15E−05 | O43683 |
| 20 | 13 | 7 | BUB1 | O43683 | wp076_MFH.5.126.1 | MFH.5.126.1 | down | −0.92479675 | 6.33E−06 | O43683 |
| 20 | 13 | 7 | BUB1 | O43683 | wp117_DB0646 | DB0646 | down | −0.34230758 | 1.15E−05 | O43683 |
| 20 | 13 | 7 | BUB1 | O43683 | wp131_TL12.186 | TL12.186 | down | −0.49826904 | 9.55E−07 | O43683 |
| 20 | 13 | 7 | BUB1 | O43683 | wp133_SK.3.91_1 hr | SK.3.91 | down | −0.49066336 | 7.75E−08 | O43683 |
| 20 | 13 | 7 | BUB1 | O43683 | wp133_SK.3.91_2 hr | SK.3.91 | down | −0.44563085 | 9.44E−08 | O43683 |
| 20 | 13 | 7 | BUB1 | O43683 | wp133_SK.3.91_4 hr | SK.3.91 | down | −0.5962723 | 8.39E−08 | O43683 |
| 20 | 13 | 7 | BUB1 | O43683 | wp134_DB118430 | DB118430 | down | −0.38664708 | 4.17E−06 | O43683 |
| 20 | 13 | 7 | BUB1 | O43683 | wp137_TL12.186 | TL12.186 | down | −0.44219835 | 1.07E−06 | O43683 |
| 20 | 13 | 7 | BUB1 | O43683 | wp152_DB0646 | DB0646 | down | −0.40139801 | 3.10E−06 | O43683 |
| 20 | 13 | 7 | BUB1 | O43683 | wp152_DB118430 | DB118430 | down | −0.38143912 | 4.10E−06 | O43683 |
| 20 | 13 | 7 | BUB1 | O43683 | wp168_DB0662 | DB0662 | down | −0.338222 | 0.00411053 | O43683 |
| 19 | 14 | 5 | MKNK2 | Q9HBH9-1 | wp045_DB0646 | DB0646 | down | −0.44630161 | 0.0019073 | Q9HBH9 |
| 19 | 14 | 5 | MKNK2 | Q9HBH9-1 | wp045_DB0663 | DB0663 | down | −0.47979903 | 0.00141905 | Q9HBH9 |
| 18 | 14 | 5 | MKNK2 | Q9HBH9-1 | wp080_RSS0680 | RSS0680 | down | −0.49865834 | 0.00512972 | Q9HBH9 |
| 19 | 14 | 5 | MKNK2 | Q9HBH9-1 | wp117_DB0646 | DB0646 | down | −0.3794128 | 0.00160981 | Q9HBH9 |
| 19 | 14 | 5 | MKNK2 | Q9HBH9-1 | wp117_RSS0680 | RSS0680 | down | −0.50745427 | 0.00029487 | Q9HBH9 |
| 19 | 14 | 5 | MKNK2 | Q9HBH9-1 | wp118_ZNL.02.096 | ZNL.02.096 | down | −0.47508536 | 0.00338788 | Q9HBH9 |
| 19 | 14 | 5 | MKNK2 | Q9HBH9-1 | wp133_SK.3.91_1 hr | SK.3.91 | down | −0.51684446 | 0.00039684 | Q9HBH9 |
| 19 | 14 | 5 | MKNK2 | Q9HBH9-1 | wp133_SK.3.91_2 hr | SK.3.91 | down | −0.53027433 | 0.00034479 | Q9HBH9 |
| 19 | 14 | 5 | MKNK2 | Q9HBH9-1 | wp133_SK.3.91_4 hr | SK.3.91 | down | −0.85374707 | 2.12E−05 | Q9HBH9 |
| 19 | 14 | 5 | MKNK2 | Q9HBH9-1 | wp160_DB0646_4 hr | DB0646 | down | −0.52262356 | 0.00488891 | Q9HBH9 |
| 19 | 14 | 5 | MKNK2 | Q9HBH9-1 | wp160_DB0646_8 hr | DB0646 | down | −0.6422345 | 0.00226654 | Q9HBH9 |
| 19 | 14 | 5 | MKNK2 | Q9HBH9-1 | wp168_DB0661 | DB0661 | down | −0.50935331 | 0.00141894 | Q9HBH9 |
| 19 | 14 | 5 | MKNK2 | Q9HBH9-1 | wp168_DB0662 | DB0662 | down | −0.41847412 | 0.00288936 | Q9HBH9 |
| 19 | 14 | 5 | MKNK2 | Q9HBH9 | wp152_DB0646 | DB0646 | down | −0.44469337 | 0.00584765 | Q9HBH9 |
| 19 | 19 | 0 | PRKAA1 | Q13131-2 | wp016_LT2.49 | LT2.49 | down | −0.37386943 | 0.00059547 | Q13131 |
| 19 | 19 | 0 | PRKAA1 | Q13131-2 | wp016_SK.3.91 | SK.3.91 | down | −0.49144282 | 0.00018805 | Q13131 |
| 19 | 19 | 0 | PRKAA1 | Q13131-2 | wp016_SK.3.91_1 uM | SK.3.91 | down | −0.79773114 | 2.35E−05 | Q13131 |
| 19 | 19 | 0 | PRKAA1 | Q13131-2 | wp016_SK.3.93 | SK.3.93 | down | −0.47382679 | 0.00021959 | Q13131 |
| 19 | 19 | 0 | PRKAA1 | Q13131-2 | wp016_TL12.186 | TL12.186 | down | −0.43395561 | 0.00031855 | Q13131 |
| 19 | 19 | 0 | PRKAA1 | Q13131-2 | wp103_SK.3.91 | SK.3.91 | down | −0.50124387 | 1.30E−05 | Q13131 |
| 19 | 19 | 0 | PRKAA1 | Q13131-2 | wp105_SK.3.91 | SK.3.91 | down | −0.44132003 | 4.16E−05 | Q13131 |
| 19 | 19 | 0 | PRKAA1 | Q13131-2 | wp107_SK.3.91 | SK.3.91 | down | −0.63520962 | 6.37E−07 | Q13131 |
| 19 | 19 | 0 | PRKAA1 | Q13131-2 | wp131_FMF.06.098.1 | FMF.06.098.1 | down | −0.36950259 | 0.00080352 | Q13131 |
| 19 | 19 | 0 | PRKAA1 | Q13131-2 | wp131_TL12.186 | TL12.186 | down | −0.59875204 | 1.67E−05 | Q13131 |
| 19 | 19 | 0 | PRKAA1 | Q13131-2 | wp133_SK.3.91_2 hr | SK.3.91 | down | −0.38042807 | 1.85E−05 | Q13131 |
| 19 | 19 | 0 | PRKAA1 | Q13131-2 | wp133_SK.3.91_4 hr | SK.3.91 | down | −0.67516973 | 2.11E−07 | Q13131 |
| 19 | 19 | 0 | PRKAA1 | Q13131-2 | wp137_TL12.186 | TL12.186 | down | −0.64099555 | 7.56E−08 | Q13131 |
| 19 | 19 | 0 | PRKAA1 | Q13131-2 | wp137_TL13.97 | TL13.97 | down | −0.66784402 | 6.02E−08 | Q13131 |
| 19 | 19 | 0 | PRKAA1 | Q13131-2 | wp146_TMX.02.138 | TMX.02.138 | down | −0.71248815 | 8.19E−05 | Q13131 |
| 19 | 19 | 0 | PRKAA1 | Q13131-2 | wp146_TMX.02.174 | TMX.02.174 | down | −1.62194747 | 4.06E−06 | Q13131 |
| 19 | 19 | 0 | PRKAA1 | Q13131 | wp160_SK.3.91_2 hr | SK.3.91 | down | −0.32841791 | 0.00023132 | Q13131 |
| 19 | 19 | 0 | PRKAA1 | Q13131 | wp160_SK.3.91_4 hr | SK.3.91 | down | −0.61170699 | 1.91E−05 | Q13131 |
| 19 | 19 | 0 | PRKAA1 | Q13131 | wp172_SK.3.91 | SK.3.91 | down | −0.45419717 | 4.72E−05 | Q13131 |
| 19 | 19 | 0 | MAP4K2 | Q12851 | wp045_DB0646 | DB0646 | down | −0.75687539 | 6.34E−05 | Q12851 |
| 19 | 19 | 0 | MAP4K2 | Q12851 | wp046_DB0663 | DB0663 | down | −0.38232613 | 0.00115663 | Q12851 |
| 19 | 19 | 0 | MAP4K2 | Q12851 | wp056_SB1.G.187 | SB1.G.187 | down | −1.84704917 | 3.51E−06 | Q12851 |
| 19 | 19 | 0 | MAP4K2 | Q12851 | wp081_TL13.150 | TL13.150 | down | −1.08831693 | 8.77E−07 | Q12851 |
| 19 | 19 | 0 | MAP4K2 | Q12851 | wp103_DB0646 | DB0646 | down | −0.46245934 | 0.00195374 | Q12851 |
| 19 | 19 | 0 | MAP4K2 | Q12851 | wp103_SB1.G.187 | SB1.G.187 | down | −1.78999045 | 4.68E−06 | Q12851 |
| 19 | 19 | 0 | MAP4K2 | Q12851 | wp105_SB1.G.187 | SB1.G.187 | down | −1.75749646 | 9.13E−06 | Q12851 |
| 19 | 19 | 0 | MAP4K2 | Q12851 | wp107_DB0646 | DB0646 | down | −0.62446274 | 4.72E−05 | Q12851 |
| 19 | 19 | 0 | MAP4K2 | Q12851 | wp107_SB1.G.187 | SB1.G.187 | down | −2.04769274 | 1.74E−07 | Q12851 |
| 19 | 19 | 0 | MAP4K2 | Q12851 | wp108_FMF.06.098.1 | FMF.06.098.1 | down | −0.55066459 | 0.00030437 | Q12851 |
| 19 | 19 | 0 | MAP4K2 | Q12851 | wp114_SB1.G.187 | SB1.G.187 | down | −0.75156139 | 0.00652134 | Q12851 |
| 19 | 19 | 0 | MAP4K2 | Q12851 | wp117_DB0646 | DB0646 | down | −0.56164303 | 9.58E−06 | Q12851 |
| 19 | 19 | 0 | MAP4K2 | Q12851 | wp131_FMF.06.098.1 | FMF.06.098.1 | down | −0.60923478 | 4.08E−06 | Q12851 |
| 19 | 19 | 0 | MAP4K2 | Q12851 | wp152_DB0646 | DB0646 | down | −0.79811195 | 3.40E−05 | Q12851 |
| 19 | 19 | 0 | MAP4K2 | Q12851 | wp152_DB1114 | DB1114 | down | −2.31305474 | 1.01E−07 | Q12851 |
| 19 | 19 | 0 | MAP4K2 | Q12851 | wp160_DB0646_4 hr | DB0646 | down | −0.6131542 | 3.98E−05 | Q12851 |
| 19 | 19 | 0 | MAP4K2 | Q12851 | wp160_DB0646_8 hr | DB0646 | down | −1.15496075 | 3.09E−06 | Q12851 |
| 19 | 19 | 0 | MAP4K2 | Q12851 | wp168_DB1113 | DB1113 | down | −2.43167312 | 2.28E−09 | Q12851 |
| 19 | 19 | 0 | MAP4K2 | Q12851 | wp195_BJG.04.203 | BJG.04.203 | down | −0.33445599 | 0.00012406 | Q12851 |
| 18 | 13 | 5 | AGK | Q53H12 | wp056_SB1.G.187 | SB1.G.187 | down | −0.57066633 | 0.00567574 | Q53H12 |
| 18 | 13 | 5 | AGK | Q53H12 | wp056_ZNL.02.096 | ZNL.02.096 | down | −0.55139462 | 0.00644436 | Q53H12 |
| 18 | 13 | 5 | AGK | Q53H12 | wp071_JWG.118 | JWG.118 | down | −0.55989103 | 0.00185619 | Q53H12 |
| 18 | 13 | 5 | AGK | Q53H12 | wp071_SB1.G.181 | SB1.G.181 | down | −0.3990903 | 0.0078083 | Q53H12 |
| 18 | 13 | 5 | AGK | Q53H12 | wp076_MFH.5.103.1 | MFH.5.103.1 | down | −0.36722208 | 0.00569437 | Q53H12 |
| 18 | 13 | 5 | AGK | Q53H12 | wp081_INY.01.140.1 | INY.01.140.1 | down | −0.49362876 | 0.00994019 | Q53H12 |
| 18 | 13 | 5 | AGK | Q53H12 | wp084_BSJ.03.123 | BSJ.03.123 | down | −0.65058575 | 0.00788691 | Q53H12 |
| 18 | 13 | 5 | AGK | Q53H12 | wp084_BSJ.04.132 | BSJ.04.132 | down | −0.670863 | 0.00192834 | Q53H12 |
| 18 | 13 | 5 | AGK | Q53H12 | wp147_DD.03.108_2 uM | DD.03.106 | down | −0.34597134 | 0.00602526 | Q53H12 |
| 18 | 13 | 5 | AGK | Q53H12 | wp147_DD.03.106_4 uM | DD.03.106 | down | −0.94990777 | 5.05E−05 | Q53H12 |
| 18 | 13 | 5 | AGK | Q53H12 | wp147_DD.03.107_1 uM | DD.03.107 | down | −0.79827517 | 0.00012027 | Q53H12 |
| 18 | 13 | 5 | AGK | Q53H12 | wp147_DD.03.107_2 uM | DD.03.107 | down | −0.86545623 | 8.04E−05 | Q53H12 |
| 18 | 13 | 5 | AGK | Q53H12 | wp147_DD.03.107_4 uM | DD.03.107 | down | −1.34335547 | 8.74E−06 | Q53H12 |
| 17 | 16 | 1 | FASTKD2 | Q9NYY8 | wp016_SK.3.91_1 uM | SK.3.91 | down | −0.45073196 | 0.00571756 | Q9NYY8 |
| 17 | 16 | 1 | FASTKD2 | Q9NYY8 | wp056_SB1.G.187 | SB1.G.187 | down | −0.38805994 | 0.00439706 | Q9NYY8 |
| 17 | 16 | 1 | FASTKD2 | Q9NYY8 | wp071_JWG.118 | JWG.118 | down | −0.43405343 | 0.00881443 | Q9NYY8 |
| 17 | 16 | 1 | FASTKD2 | Q9NYY8 | wp081_INY.01.140.1 | INY.01.140.1 | down | −0.40534105 | 6.35E−05 | Q9NYY8 |
| 17 | 16 | 1 | FASTKD2 | Q9NYY8 | wp081_SB1.G.192.1 | SB1.G.192.1 | down | −0.35771434 | 0.00012298 | Q9NYY8 |
| 17 | 16 | 1 | FASTKD2 | Q9NYY8 | wp084_BSJ.03.123 | BSJ.03.123 | down | −0.50998137 | 0.00033868 | Q9NYY8 |
| 17 | 16 | 1 | FASTKD2 | Q9NYY8 | wp084_BSJ.04.132 | BSJ.04.132 | down | −0.51071251 | 0.00054078 | Q9NYY8 |
| 17 | 16 | 1 | FASTKD2 | Q9NYY8 | wp118_INY.03.041.01 | INY.03.041.01 | down | −0.45393372 | 0.00416256 | Q9NYY8 |
| 17 | 16 | 1 | FASTKD2 | Q9NYY8 | wp118_ZNL.02.096 | ZNL.02.096 | down | −0.46053615 | 0.00393662 | Q9NYY8 |
| 17 | 16 | 1 | FASTKD2 | Q9NYY8 | wp137_TL12.186 | TL12.186 | down | −0.36245817 | 0.00010792 | Q9NYY8 |
| 17 | 16 | 1 | FASTKD2 | Q9NYY8 | wp147_DD.03.106_2 uM | DD.03.106 | down | −0.33918433 | 0.00439091 | Q9NYY8 |
| 17 | 16 | 1 | FASTKD2 | Q9NYY8 | wp147_DD.03.108_4 uM | DD.03.106 | down | −0.90727941 | 3.98E−05 | Q9NYY8 |
| 17 | 16 | 1 | FASTKD2 | Q9NYY8 | wp147_DD.03.107_1 uM | DD.03.107 | down | −0.72361648 | 0.00012319 | Q9NYY8 |
| 17 | 16 | 1 | FASTKD2 | Q9NYY8 | wp147_DD.03.107_2 uM | DD.03.107 | down | −0.67835635 | 0.00016956 | Q9NYY8 |
| 17 | 16 | 1 | FASTKD2 | Q9NYY8 | wp147_DD.03.107_4 uM | DD.03.107 | down | −1.27267945 | 7.15E−06 | Q9NYY8 |
| 17 | 16 | 1 | FASTKD2 | Q9NYY8 | wp160_SK.3.91_1 hr | SK.3.91 | down | −1.00037789 | 0.00260195 | Q9NYY8 |
| 17 | 14 | 3 | PFKFB3 | Q16875-4 | wp117_DB0646 | DB0646 | down | −0.37695847 | 9.71E−05 | Q16875 |
| 17 | 14 | 3 | PFKFB3 | Q16875 | wp045_DB0646 | DB0646 | down | −0.5182126 | 0.0007716 | Q16875 |
| 17 | 14 | 3 | PFKFB3 | Q16875 | wp045_DB0663 | DB0663 | down | −0.4221309 | 0.00179923 | Q16875 |
| 17 | 14 | 3 | PFKFB3 | Q16875 | wp068_FMF.04.147.1 | FMF.04.147.1 | down | −0.4462123 | 0.00611957 | Q16875 |
| 17 | 14 | 3 | PFKFB3 | Q16875 | wp118_ZNL.02.096 | ZNL.02.096 | down | −0.40506608 | 0.00140484 | Q16875 |
| 17 | 14 | 3 | PFKFB3 | Q16875 | wp131_TL12.186 | TL12.186 | down | −0.56434603 | 0.0003765 | Q16875 |
| 17 | 14 | 3 | PFKFB3 | Q16875 | wp133_SK.3.91_2 hr | SK.3.91 | down | −0.35388042 | 3.49E−05 | Q16875 |
| 17 | 14 | 3 | PFKFB3 | Q16875 | wp133_SK.3.91_4 hr | SK.3.91 | down | −0.60353877 | 1.77E−06 | Q16875 |
| 17 | 14 | 3 | PFKFB3 | Q16875 | wp134_DB118430 | DB118430 | down | −0.46155171 | 7.45E−06 | Q16875 |
| 17 | 14 | 3 | PFKFB3 | Q16875 | wp137_TL12.186 | TL12.186 | down | −0.40667468 | 2.72E−05 | Q16875 |
| 17 | 14 | 3 | PFKFB3 | Q16875 | wp160_DB0646_8 hr | DB0646 | down | −0.37540364 | 0.00137544 | Q16875 |
| 17 | 14 | 3 | PFKFB3 | Q16875 | wp168_DB0661 | DB0661 | down | −0.43924148 | 7.78E−05 | Q16875 |
| 17 | 14 | 3 | PFKFB3 | Q16875 | wp168_DB0662 | DB0662 | down | −0.45170018 | 5.49E−06 | Q16875 |
| 17 | 14 | 3 | PFKFB3 | Q16875 | wp168_DB1113 | DB1113 | down | −0.37235169 | 1.95E−05 | Q16875 |
| 17 | 12 | 5 | PDK2 | Q15119-1 | wp056_SB1.G.187 | SB1.G.187 | down | −0.40158089 | 0.00693982 | Q15119 |
| 17 | 12 | 5 | PDK2 | Q15119-1 | wp056_ZNL.02.096 | ZNL.02.096 | down | −0.55608151 | 0.00202246 | Q15119 |
| 17 | 12 | 5 | PDK2 | Q15119-1 | wp081_INY.01.140.1 | INY.01.140.1 | down | −0.60944321 | 0.00656283 | Q15119 |
| 17 | 12 | 5 | PDK2 | Q15119-1 | wp084_BS.J.03.123 | BSJ.03.123 | down | −0.3615241 | 0.0038807 | Q15119 |
| 17 | 12 | 5 | PDK2 | Q15119-1 | wp084_BSJ.04.132 | BSJ.04.132 | down | −0.52891927 | 0.00015271 | Q15119 |
| 17 | 12 | 5 | PDK2 | Q15119-1 | wp118_ZNL.02.096 | ZNL.02.096 | down | −0.37003418 | 0.00602864 | Q15119 |
| 17 | 12 | 5 | PDK2 | Q15119-1 | wp137_DD.03.107 | DD.03.107 | down | −0.33827147 | 0.00291816 | Q15119 |
| 17 | 12 | 5 | PDK2 | Q15119-1 | wp137_TL12.186 | TL12.186 | down | −0.50219806 | 0.00041279 | Q15119 |
| 17 | 12 | 5 | PDK2 | Q15119-1 | wp147_DD.03.106_4 uM | DD.03.108 | down | −0.72699052 | 0.00022056 | Q15119 |
| 17 | 12 | 5 | PDK2 | Q15119-1 | wp147_DD.03.107_1 uM | DD.03.107 | down | −0.49632833 | 0.00137233 | Q15119 |
| 17 | 12 | 5 | PDK2 | Q15119-1 | wp147_DD.03.107_2 uM | DD.03.107 | down | −0.53507708 | 0.00096548 | Q15119 |
| 17 | 12 | 5 | PDK2 | Q15119-1 | wp147_DD.03.107_4 uM | DD.03.107 | down | −0.92690348 | 6.61E−05 | Q15119 |
| 17 | 17 | 0 | CDK13 | Q14004-1 | wp016_LT2.49 | LT2.49 | down | −0.38389819 | 0.00099483 | Q14004 |
| 17 | 17 | 0 | CDK13 | Q14004-1 | wp016_SK.3.91 | SK.3.91 | down | −0.60456247 | 0.00014756 | Q14004 |
| 17 | 17 | 0 | CDK13 | Q14004-1 | wp016_SK.3.91_1 uM | SK.3.91 | down | −0.5748761 | 0.00018285 | Q14004 |
| 17 | 17 | 0 | CDK13 | Q14004-1 | wp016_SK.3.93 | SK.3.93 | down | −0.5636444 | 0.00019885 | Q14004 |
| 17 | 17 | 0 | CDK13 | Q14004-1 | wp016_TL12.186 | TL12.186 | down | −0.58849123 | 0.00016551 | Q14004 |
| 17 | 17 | 0 | CDK13 | Q14004-1 | wp076_MFH.6.126.1 | MFH.5.126.1 | down | −0.48464674 | 0.00065037 | Q14004 |
| 17 | 17 | 0 | CDK13 | Q14004-1 | wp103_SK.3.91 | SK.3.91 | down | −0.48329938 | 3.57E−05 | Q14004 |
| 17 | 17 | 0 | CDK13 | Q14004-1 | wp105_SK.3.91 | SK.3.91 | down | −0.46486682 | 1.29E−05 | Q14004 |
| 17 | 17 | 0 | CDK13 | Q14004-1 | wp107_SK.3.91 | SK.3.91 | down | −0.71119191 | 4.74E−07 | Q14004 |
| 17 | 17 | 0 | CDK13 | Q14004-1 | wp108_FMF.06.098.1 | FMF.06.098.1 | down | −0.36641062 | 6.64E−05 | Q14004 |
| 17 | 17 | 0 | CDK13 | Q14004-1 | wp131_FMF.06.098.1 | FMF.06.098.1 | down | −0.49813319 | 5.48E−07 | Q14004 |
| 17 | 17 | 0 | CDK13 | Q14004-1 | wp131_TL12.186 | TL12.186 | down | −0.79030456 | 6.16E−07 | Q14004 |
| 17 | 17 | 0 | CDK13 | Q14004-1 | wp133_SK.3.91_4 hr | SK.3.91 | down | −0.55623544 | 3.44E−06 | Q14004 |
| 17 | 17 | 0 | CDK13 | Q14004-1 | wp137_TL12.186 | TL12.186 | down | −0.70620737 | 5.46E−07 | Q14004 |
| 17 | 17 | 0 | CDK13 | Q14004-1 | wp137_TL13.97 | TL13.97 | down | −0.73594346 | 4.34E−07 | Q14004 |
| 17 | 17 | 0 | CDK13 | Q14004-1 | wp160_SK.3.91_4 hr | SK.3.91 | down | −0.48050778 | 0.00010019 | Q14004 |
| 17 | 17 | 0 | CDK13 | Q14004-1 | wp172_SK.3.91 | SK.3.91 | down | −0.85900345 | 3.21E−06 | Q14004 |
| 17 | 15 | 2 | ALDH18A1 | P54886 | wp056_SB1.G.187 | SB1.G.187 | down | −0.44577693 | 0.00089474 | P54886 |
| 17 | 15 | 2 | ALDH18A1 | P54886 | wp071_JWG.118 | JWG.118 | down | −0.42267657 | 0.00363218 | P54886 |
| 17 | 15 | 2 | ALDH18A1 | P54886 | wp076_MFH.5.103.1 | MFH.5.103.1 | down | −0.41911873 | 0.00362831 | P54886 |
| 17 | 15 | 2 | ALDH18A1 | P54886 | wp081_INY.01.140.1 | INY.01.140.1 | down | −0.44907609 | 7.25E−06 | P54886 |
| 17 | 15 | 2 | ALDH18A1 | P54886 | wp081_SB1.G.192.1 | SB1.G.192.1 | down | −0.39002671 | 1.56E−05 | P54886 |
| 17 | 15 | 2 | ALDH18A1 | P54886 | wp084_BSJ.03.123 | BSJ.03.123 | down | −0.52822767 | 0.00092703 | P54886 |
| 17 | 15 | 2 | ALDH18A1 | P54886 | wp084_BSJ.04.132 | BSJ.04.132 | down | −0.65681928 | 7.07E−05 | P54886 |
| 17 | 15 | 2 | ALDH18A1 | P54886 | wp118_INY.03.041.01 | INY.03.041.01 | down | −0.37598361 | 0.00606146 | P54886 |
| 17 | 15 | 2 | ALDH18A1 | P54886 | wp118_ZNL.02.096 | ZNL.02.096 | down | −0.44049954 | 0.00329844 | P54886 |
| 17 | 15 | 2 | ALDH18A1 | P54886 | wp147_DD.03.106_1 uM | DD.03.106 | down | −0.34633581 | 0.00201122 | P54886 |
| 17 | 15 | 2 | ALDH18A1 | P54886 | wp147_DD.03.106_4 uM | DD.03.106 | down | −0.88232216 | 2.10E−05 | P54886 |
| 17 | 15 | 2 | ALDH18A1 | P54886 | wp147_DD.03.107_1 uM | DD.03.107 | down | −0.65359939 | 9.50E−05 | P54886 |
| 17 | 15 | 2 | ALDH18A1 | P54886 | wp147_DD.03.107_2 uM | DD.03.107 | down | −0.73738001 | 5.20E−05 | P54886 |
| 17 | 15 | 2 | ALDH18A1 | P54886 | wp147_DD.03.107_4 uM | DD.03.107 | down | −1.32604052 | 2.64E−06 | P54886 |
| 17 | 15 | 2 | ALDH18A1 | P54886 | wp160_SK.3.91_1 hr | SK.3.91 | down | −0.96836331 | 0.00452882 | P54886 |
| 17 | 16 | 1 | TTK | P33981 | wp045_DB0646 | DB0646 | down | −0.57574741 | 4.71E−05 | P33981 |
| 17 | 16 | 1 | TTK | P33981 | wp076_MFH.5.115.2 | MFH.5.115.2 | down | −0.32486785 | 0.00347607 | P33981 |
| 17 | 16 | 1 | TTK | P33981 | wp076_MFH.5.126.1 | MFH.5.126.1 | down | −0.44123116 | 0.00099642 | P33981 |
| 17 | 16 | 1 | TTK | P33981 | wp103_SK.3.91 | SK.3.91 | down | −0.32667459 | 0.00136234 | P33981 |
| 17 | 16 | 1 | TTK | P33981 | wp107_DB0646 | DB0646 | down | −0.37196466 | 0.00051481 | P33981 |
| 17 | 16 | 1 | TTK | P33981 | wp107_SK.3.91 | SK.3.91 | down | −0.47806801 | 0.00044944 | P33981 |
| 17 | 16 | 1 | TTK | P33981 | wp117_DB0646 | DB0646 | down | −0.42669711 | 1.37E−05 | P33981 |
| 17 | 16 | 1 | TTK | P33981 | wp133_SK.3.91_2 hr | SK.3.91 | down | −0.38334486 | 2.65E−05 | P33981 |
| 17 | 16 | 1 | TTK | P33981 | wp133_SK.3.91_4 hr | SK.3.91 | down | −0.50515028 | 2.03E−06 | P33981 |
| 17 | 16 | 1 | TTK | P33981 | wp146_TMX.02.138 | TMX.02.138 | down | −0.61776087 | 0.00567339 | P33981 |
| 17 | 16 | 1 | TTK | P33981 | wp146_TMX.02.172 | TMX.02.172 | down | −0.75421481 | 0.00286767 | P33981 |
| 17 | 16 | 1 | TTK | P33981 | wp146_TMX.02.174 | TMX.02.174 | down | −0.72251192 | 0.0033261 | P33981 |
| 17 | 16 | 1 | TTK | P33981 | wp152_DB0646 | DB0646 | down | −0.51306578 | 5.99E−06 | P33981 |
| 17 | 16 | 1 | TTK | P33981 | wp160_DB0646_4 hr | DB0646 | down | −0.51298414 | 0.00010419 | P33981 |
| 17 | 16 | 1 | TTK | P33981 | wp160_DB0646_8 hr | DB0646 | down | −0.70566944 | 2.90E−05 | P33981 |
| 17 | 16 | 1 | TTK | P33981 | wp164_SB1.G.194 | SB1.G.194 | down | −0.42794775 | 1.26E−05 | P33981 |
| 15 | 15 | 0 | FYN | P06241-2 | wp047_DD.03.119 | DD.03.119 | down | −0.86689585 | 9.33E−05 | P06241 |
| 15 | 15 | 0 | FYN | P06241-2 | wp071_SB1.G.181 | SB1.G.181 | down | −2.27590516 | 9.12E−07 | P06241 |
| 15 | 15 | 0 | FYN | P06241-2 | wp071_SB1.G.192.1 | SB1.G.192.1 | down | −1.59494889 | 5.18E−06 | P06241 |
| 15 | 15 | 0 | FYN | P06241-2 | wp071_SB1.G.200 | SB1.G.200 | down | −1.77731876 | 3.05E−06 | P06241 |
| 15 | 15 | 0 | FYN | P06241-2 | wp081_SB1.G.192.1 | SB1.G.192.1 | down | −0.99940861 | 5.46E−05 | P06241 |
| 15 | 15 | 0 | FYN | P06241-2 | wp107_DB0646 | DB0646 | down | −0.39988351 | 0.00060897 | P06241 |
| 15 | 15 | 0 | FYN | P06241-2 | wp107_SB1.G.187 | SB1.G.187 | down | −0.53336148 | 0.00028016 | P06241 |
| 15 | 15 | 0 | FYN | P06241-2 | wp131_SB1.G.192.1 | SB1.G.192.1 | down | −0.99178004 | 1.65E−07 | P06241 |
| 15 | 15 | 0 | FYN | P06241-2 | wp160_DB0646_4 hr | DB0646 | down | −0.39218345 | 0.0006135 | P06241 |
| 15 | 15 | 0 | FYN | P06241-2 | wp168_DB0662 | DB0662 | down | −0.56359321 | 0.00955128 | P06241 |
| 15 | 15 | 0 | FYN | P06241-1 | wp081_SB1.G.192.1 | SB1.G.192.1 | down | −0.93142497 | 0.00167881 | P06241 |
| 15 | 15 | 0 | FYN | P06241-1 | wp105_DB0646 | DB0646 | down | −0.46214066 | 0.00063573 | P06241 |
| 15 | 15 | 0 | FYN | P06241-1 | wp105_SB1.G.187 | SB1.G.187 | down | −0.76967344 | 0.00018087 | P06241 |
| 15 | 15 | 0 | FYN | P06241-1 | wp105_SK.3.91 | SK.3.91 | down | −0.35863169 | 0.00348289 | P06241 |
| 15 | 15 | 0 | FYN | P06241-1 | wp107_SB1.G.187 | SB1.G.187 | down | −0.36006468 | 0.0030935 | P06241 |
| 15 | 15 | 0 | FYN | P06241-1 | wp107_SK.3.91 | SK.3.91 | down | −0.4288427 | 0.00054561 | P06241 |
| 15 | 15 | 0 | FYN | P06241 | wp152_DB0646 | DB0646 | down | −0.36849132 | 2.80E−05 | P06241 |
| 16 | 13 | 3 | IP6K2 | Q9UHH9 | wp016_SK.3.91_1 uM | SK.3.91 | down | −0.93681842 | 0.00264825 | Q9UHH9 |
| 16 | 13 | 3 | IP6K2 | Q9UHH9 | wp107_SK.3.91 | SK.3.91 | down | −1.47205423 | 1.85E−05 | Q9UHH9 |
| 16 | 13 | 3 | IP6K2 | Q9UHH9 | wp118_INY.03.041.01 | INY.03.041.01 | down | −0.66198861 | 0.00269257 | Q9UHH9 |
| 16 | 13 | 3 | IP6K2 | Q9UHH9 | wp133_SK.3.91_1 hr | SK.3.91 | down | −0.45474169 | 0.00402574 | Q9UHH9 |
| 16 | 13 | 3 | IP6K2 | Q9UHH9 | wp133_SK.3.91_2 hr | SK.3.91 | down | −0.64605503 | 0.00066625 | Q9UHH9 |
| 16 | 13 | 3 | IP6K2 | Q9UHH9 | wp133_SK.3.91_4 hr | SK.3.91 | down | −0.96922979 | 7.72E−05 | Q9UHH9 |
| 16 | 13 | 3 | IP6K2 | Q9UHH9 | wp135_INY.03.041_01_8 hr | INY.03.041_01 | down | −0.6455505 | 9.51E−05 | Q9UHH9 |
| 16 | 13 | 3 | IP6K2 | Q9UHH9 | wp135_INY.03.041.01_4 hr | INY.03.041.01 | down | −0.3250681 | 0.00476324 | Q9UHH9 |
| 16 | 13 | 3 | IP6K2 | Q9UHH9 | wp137_INY.02.164 | INY.02.164 | down | −0.544897 | 0.00157051 | Q9UHH9 |
| 16 | 13 | 3 | IP6K2 | Q9UHH9 | wp137_TL12.186 | TL12.186 | down | −0.75384098 | 0.00030488 | Q9UHH9 |
| 16 | 13 | 3 | IP6K2 | Q9UHH9 | wp160_SK.3.91_2 hr | SK.3.91 | down | −0.49370888 | 0.00526034 | Q9UHH9 |
| 16 | 13 | 3 | IP6K2 | Q9UHH9 | wp160_SK.3.91_4 hr | SK.3.91 | down | −0.62809763 | 0.00214417 | Q9UHH9 |
| 16 | 13 | 3 | IP6K2 | Q9UHH9 | wp195_INY.04.125.01 | INY.04.125.01 | down | −0.49462453 | 0.00023729 | Q9UHH9 |
| 16 | 16 | 0 | CDK12 | Q9NYV4-2 | wp107_SK.3.91 | SK.3.91 | down | −1.04204295 | 3.48E−08 | Q9NYV4 |
| 16 | 16 | 0 | CDK12 | Q9NYV4-2 | wp160_SK.3.91_2 hr | SK.3.91 | down | −0.45578445 | 1.69E−05 | Q9NYV4 |
| 16 | 16 | 0 | CDK12 | Q9NYV4-2 | wp160_SK.3.91_4 hr | SK.3.91 | down | −0.84475965 | 1.39E−06 | Q9NYV4 |
| 16 | 16 | 0 | CDK12 | Q9NYV4-1 | wp016_LT2.49 | LT2.49 | down | −0.86801827 | 8.54E−06 | Q9NYV4 |
| 16 | 16 | 0 | CDK12 | Q9NYV4-1 | wp016_SK.3.87 | SK.3.87 | down | −0.50727729 | 8.68E−05 | Q9NYV4 |
| 16 | 16 | 0 | CDK12 | Q9NYV4-1 | wp016_SK.3.89 | SK.3.89 | down | −0.52369765 | 7.57E−05 | Q9NYV4 |
| 16 | 16 | 0 | CDK12 | Q9NYV4-1 | wp016_SK.3.91 | SK.3.91 | down | −1.13402498 | 2.67E−06 | Q9NYV4 |
| 16 | 16 | 0 | CDK12 | Q9NYV4-1 | wp016_SK.3.91_1 uM | SK.3.91 | down | −0.9324763 | 6.26E−06 | Q9NYV4 |
| 16 | 16 | 0 | CDK12 | Q9NYV4-1 | wp016_SK.3.93 | SK.3.93 | down | −0.95552302 | 5.63E−06 | Q9NYV4 |
| 16 | 16 | 0 | CDK12 | Q9NYV4-1 | wp016_TL12.186 | TL12.186 | down | −0.91850601 | 6.68E−06 | Q9NYV4 |
| 16 | 16 | 0 | CDK12 | Q9NYV4-1 | wp131_FMF.06.098.1 | FMF.06.098.1 | down | −0.57318899 | 6.12E−05 | Q9NYV4 |
| 16 | 16 | 0 | CDK12 | Q9NYV4-1 | wp131_TL12.186 | TL12.186 | down | −1.40211306 | 5.31E−07 | Q9NYV4 |
| 16 | 16 | 0 | CDK12 | Q9NYV4-1 | wp146_TMX.01.180 | TMX.01.160 | down | −0.51178924 | 0.00081768 | Q9NYV4 |
| 16 | 16 | 0 | CDK12 | Q9NYV4-1 | wp146_TMX.02.138 | TMX.02.138 | down | −1.01808477 | 6.83E−05 | Q9NYV4 |
| 16 | 16 | 0 | CDK12 | Q9NYV4-1 | wp146_TMX.02.174 | TMX.02.174 | down | −0.85621541 | 0.00012813 | Q9NYV4 |
| 16 | 16 | 0 | CDK12 | Q9NYV4-1 | wp172_SK.3.91 | SK.3.91 | down | −1.23437092 | 2.32E−07 | Q9NYV4 |
| 16 | 16 | 0 | MARK4 | Q96L34-1 | wp080_RSS0680 | RSS0680 | down | −0.45370195 | 0.00467195 | Q96L34 |
| 16 | 16 | 0 | MARK4 | Q96L34-1 | wp103_SK.3.91 | SK.3.91 | down | −1.32645784 | 0.00107408 | Q96L34 |
| 16 | 16 | 0 | MARK4 | Q96L34-1 | wp107_SK.3.91 | SK.3.91 | down | −1.29155966 | 0.0002121 | Q96L34 |
| 16 | 16 | 0 | MARK4 | Q96L34-1 | wp108_FMF.06.098.1 | FMF.06.098.1 | down | −0.90413066 | 0.00010729 | Q96L34 |
| 16 | 16 | 0 | MARK4 | Q96L34-1 | wp114_FMF.06.098.1 | FMF.06.098.1 | down | −0.72244783 | 0.00428822 | Q96L34 |
| 16 | 16 | 0 | MARK4 | Q96L34-1 | wp114_SK.3.91 | SK.3.91 | down | −1.30007777 | 0.00044692 | Q96L34 |
| 16 | 16 | 0 | MARK4 | Q96L34-1 | wp131_FMF.06.098.1 | FMF.06.098.1 | down | −1.11065666 | 4.39E−05 | Q96L34 |
| 16 | 16 | 0 | MARK4 | Q96L34-1 | wp133_SK.3.91_2 hr | SK.3.91 | down | −0.62819225 | 0.00514444 | Q96L34 |
| 16 | 16 | 0 | MARK4 | Q96L34-1 | wp133_SK.3.91_4 hr | SK.3.91 | down | −1.53710472 | 5.37E−05 | Q96L34 |
| 16 | 16 | 0 | MARK4 | Q96L34-1 | wp134_BSJ.04.178 | BSJ.04.178 | down | −0.99298918 | 0.00016129 | Q96L34 |
| 16 | 16 | 0 | MARK4 | Q96L34-1 | wp137_TL12.186 | TL12.186 | down | −1.29787793 | 3.93E−05 | Q96L34 |
| 16 | 16 | 0 | MARK4 | Q96L34-1 | wp137_TL13.97 | TL13.97 | down | −1.73980818 | 7.96E−06 | Q96L34 |
| 16 | 16 | 0 | MARK4 | Q96L34-1 | wp160_SK.3.91_4 hr | SK.3.91 | down | −1.52875963 | 0.0003236 | Q96L34 |
| 16 | 16 | 0 | MARK4 | Q96L34-1 | wp195_BSJ.05.026 | BSJ.05.026 | down | −1.57080633 | 2.56E−07 | Q96L34 |
| 16 | 16 | 0 | MARK4 | Q96L34-1 | wp195_JWG.123 | JWG.123 | down | −1.53223045 | 2.94E−07 | Q96L34 |
| 16 | 13 | 3 | FASTKD5 | Q7L8L6 | wp056_SB1.G.187 | SB1.G.187 | down | −0.423793 | 0.00580336 | Q7L8L6 |
| 16 | 13 | 3 | FASTKD5 | Q7L8L6 | wp076_MFH.5.103.1 | MFH.5.103.1 | down | −0.34932527 | 0.00557921 | Q7L8L6 |
| 16 | 13 | 3 | FASTKD5 | Q7L8L6 | wp081_INY.01.140.1 | INY.01.140.1 | down | −0.41821486 | 0.00011663 | Q7L8L6 |
| 16 | 13 | 3 | FASTKD5 | Q7L8L6 | wp084_BSJ.03.123 | BSJ.03.123 | down | −0.59258924 | 0.00038442 | Q7L8L6 |
| 16 | 13 | 3 | FASTKD5 | Q7L8L6 | wp084_BSJ.04.132 | BSJ.04.132 | down | −0.70733845 | 5.14E−05 | Q7L8L6 |
| 16 | 13 | 3 | FASTKD5 | Q7L8L6 | wp118_ZNL.02.096 | ZNL.02.096 | down | −0.45515411 | 0.00192 | Q7LBL6 |
| 16 | 13 | 3 | FASTKD5 | Q7L8L6 | wp137_INY.02.164 | INY.02.164 | down | −0.34224786 | 0.00014889 | Q7L8L6 |
| 16 | 13 | 3 | FASTKD5 | Q7L8L6 | wp137_TL12.186 | TL12.186 | down | −0.34092643 | 0.00015196 | Q7L8L6 |
| 16 | 13 | 3 | FASTKD5 | Q7L8L6 | wp147_DD.03.106_4 uM | DD.03.106 | down | −0.86882542 | 5.65E−05 | Q7L8L6 |
| 16 | 13 | 3 | FASTKD5 | Q7L8L6 | wp147_DD.03.107_1 uM | DD.03.107 | down | −0.59845506 | 0.00035682 | Q7L8L6 |
| 16 | 13 | 3 | FASTKD5 | Q7L8L6 | wp147_DD.03.107_2 uM | DD.03.107 | down | −0.68393776 | 0.00018572 | Q7L8L6 |
| 16 | 13 | 3 | FASTKD5 | Q7L8L6 | wp147_DD.03.107_4 uM | DD.03.107 | down | −1.26818226 | 8.34E−06 | Q7L8L6 |
| 16 | 13 | 3 | FASTKD5 | Q7L8L6 | wp160_SK.3.91_1 hr | SK.3.91 | down | −0.83061136 | 0.00340922 | Q7L8L6 |
| 16 | 14 | 2 | FASTKD1 | Q53R41 | wp056_SB1.G.187 | SB1.G.187 | down | −0.48105642 | 0.00194812 | Q53R41 |
| 16 | 14 | 2 | FASTKD1 | Q53R41 | wp056_ZNL.02.096 | ZNL.02.096 | down | −0.40792001 | 0.00367548 | Q53R41 |
| 16 | 14 | 2 | FASTKD1 | Q53R41 | wp081_INY.01.140.1 | INY.01.140.1 | down | −0.48077995 | 0.00128837 | Q53R41 |
| 16 | 14 | 2 | FASTKD1 | Q53R41 | wp081_SB1.G.192.1 | SB1.G.192.1 | down | −0.4221764 | 0.00240872 | Q53R41 |
| 16 | 14 | 2 | FASTKD1 | Q53R41 | wp084_BSJ.03.123 | BSJ.03.123 | down | −0.52421557 | 0.00080494 | Q53R41 |
| 16 | 14 | 2 | FASTKD1 | Q53R41 | wp084_BSJ.04.132 | BSJ.04.132 | down | −0.67468341 | 0.00011504 | Q53R41 |
| 16 | 14 | 2 | FASTKD1 | Q53R41 | wp118_ZNL.02.096 | ZNL.02.096 | down | −0.45594821 | 0.00333762 | Q53R41 |
| 16 | 14 | 2 | FASTKD1 | Q53R41 | wp137_TL12.186 | TL12.186 | down | −0.37311213 | 0.00055869 | Q53R41 |
| 16 | 14 | 2 | FASTKD1 | Q53R41 | wp137_TL13.97 | TL13.97 | down | −0.368425 | 0.00059584 | Q53R41 |
| 16 | 14 | 2 | FASTKD1 | Q53R41 | wp147_DD.03.106_4 uM | DD.03.106 | down | −0.6261929 | 0.00087969 | Q53R41 |
| 16 | 14 | 2 | FASTKD1 | Q53R41 | wp147_DD.03.107_1 uM | DD.03.107 | down | −0.57997678 | 0.00126075 | Q53R41 |
| 16 | 14 | 2 | FASTKD1 | Q53R41 | wp147_DD.03.107_2 uM | DD.03.107 | down | −0.50437818 | 0.00239455 | Q53R41 |
| 16 | 14 | 2 | FASTKD1 | Q53R41 | wp147_DD.03.107_4 uM | DD.03.107 | down | −0.9447423 | 0.00011932 | Q53R41 |
| 16 | 14 | 2 | FASTKD1 | Q53R41 | wp160_SK.3.91_1 hr | SK.3.91 | down | −0.86753449 | 0.00636887 | Q53R41 |
| 16 | 11 | 5 | PDK3 | Q15120-2 | wp056_SB1.G.187 | SB1.G.187 | down | −0.48266911 | 0.00135603 | Q15120 |
| 16 | 11 | 5 | PDK3 | Q15120-2 | wp056_ZNL.02.096 | ZNL.02.096 | down | −0.35275431 | 0.00454507 | Q15120 |
| 16 | 11 | 5 | PDK3 | Q15120-2 | wp081_INY.01.140.1 | INY.01.140.1 | down | −0.37550453 | 0.00144924 | Q15120 |
| 16 | 11 | 5 | PDK3 | Q15120-2 | wp084_BSJ.03.123 | BSJ.03.123 | down | −0.59081871 | 0.00073438 | Q15120 |
| 16 | 11 | 5 | PDK3 | Q15120-2 | wp084_BS.J.04.132 | BSJ.04.132 | down | −0.73965682 | 0.00014378 | Q15120 |
| 16 | 11 | 5 | PDK3 | Q15120-2 | wp137_DD.03.106 | DD.03.106 | down | −0.35705584 | 0.00042399 | Q15120 |
| 16 | 11 | 5 | PDK3 | Q15120-2 | wp147_DD.03.106_4 uM | DD.03.108 | down | −0.65644782 | 9.79E−05 | Q15120 |
| 16 | 11 | 5 | PDK3 | Q15120-2 | wp147_DD.03.107_1 uM | DD.03.107 | down | −0.51187908 | 0.00033331 | Q15120 |
| 16 | 11 | 5 | PDK3 | Q15120-2 | wp147_DD.03.107_2 uM | DD.03.107 | down | −0.54063924 | 0.0002553 | Q15120 |
| 16 | 11 | 5 | PDK3 | Q15120-2 | wp147_DD.03.107_4 uM | DD.03.107 | down | −1.00415066 | 1.15E−05 | Q15120 |
| 16 | 11 | 5 | PDK3 | Q15120-1 | wp160_SK.3.91_1 hr | SK.3.91 | down | −0.98014782 | 0.00652953 | Q15120 |
| 16 | 16 | 0 | RIPK1 | Q13546 | wp045_DB0646 | DB0646 | down | −0.71153202 | 8.05E−06 | Q13546 |
| 16 | 16 | 0 | RIPK1 | Q13546 | wp056_SB1.G.187 | SB1.G.187 | down | −1.2881628 | 2.07E−06 | Q13546 |
| 16 | 16 | 0 | RIPK1 | Q13546 | wp081_TL13.150 | TL13.150 | down | −0.66700857 | 1.05E−06 | Q13546 |
| 16 | 16 | 0 | RIPK1 | Q13546 | wp103_DB0646 | DB0646 | down | −0.47960073 | 3.98E−05 | Q13546 |
| 16 | 16 | 0 | RIPK1 | Q13546 | wp103_SB1.G.187 | SB1.G.187 | down | −1.76675107 | 1.02E−07 | Q13546 |
| 16 | 16 | 0 | RIPK1 | Q13546 | wp105_DB0646 | DB0646 | down | −0.42329302 | 2.40E−05 | Q13546 |
| 16 | 16 | 0 | RIPK1 | Q13546 | wp105_SB1.G.187 | SB1.G.187 | down | −1.14124949 | 8.06E−07 | Q13546 |
| 16 | 16 | 0 | RIPK1 | Q13546 | wp107_DB0646 | DB0646 | down | −0.51209968 | 4.97E−06 | Q13546 |
| 16 | 16 | 0 | RIPK1 | Q13546 | wp107_SB1.G.187 | SB1.G.187 | down | −1.50183728 | 2.71E−08 | Q13546 |
| 18 | 16 | 0 | RIPK1 | Q13546 | wp117_DB0646 | DB0646 | down | −0.61602281 | 1.05E−06 | Q13546 |
| 16 | 16 | 0 | RIPK1 | Q13546 | wp152_DB0646 | DB0646 | down | −0.54951438 | 8.14E−07 | Q13546 |
| 16 | 16 | 0 | RIPK1 | Q13546 | wp152_DB1114 | DB1114 | down | −2.2789337 | 3.14E−10 | Q13546 |
| 16 | 16 | 0 | RIPK1 | Q13546 | wp160_DB0646_4 hr | DB0646 | down | −0.39073698 | 4.17E−05 | Q13546 |
| 16 | 16 | 0 | RIPK1 | Q13546 | wp160_DB0646_8 hr | DB0646 | down | −0.8745367 | 1.61E−06 | Q13546 |
| 16 | 16 | 0 | RIPK1 | Q13546 | wp168_DB1113 | DB1113 | down | −1.9895527 | 5.92E−10 | Q13546 |
| 16 | 16 | 0 | RIPK1 | Q13546 | wp172_SB1.G.187 | SB1.G.187 | down | −0.96661365 | 4.34E−06 | Q13546 |
| 16 | 16 | 0 | CDK16 | Q00536-2 | wp080_RSS0680 | RSS0680 | down | −1.0017077 | 0.00311245 | Q00536 |
| 16 | 16 | 0 | CDK16 | Q00536-2 | wp104_TMX.01.160.1_0.25 | TMX.01.160.1 | down | −0.86463002 | 2.77E−06 | Q00536 |
| 16 | 16 | 0 | CDK16 | Q00536-2 | wp104_TMX.01.160.1_1 | TMX.01.160.1 | down | −0.60094977 | 1.75E−05 | Q00536 |
| 16 | 16 | 0 | CDK16 | Q00536-2 | wp105_SK.3.91 | SK.3.91 | down | −0.63455228 | 0.00069211 | Q00536 |
| 16 | 16 | 0 | CDK16 | Q00536-2 | wp107_SK.3.91 | SK.3.91 | down | −0.47005614 | 0.00429096 | Q00536 |
| 16 | 16 | 0 | CDK16 | Q00536-2 | wp117_DB0614 | DB0614 | down | −0.55281029 | 0.00330184 | Q00536 |
| 16 | 16 | 0 | CDK16 | Q00536-2 | wp117_RSS0680 | RSS0680 | down | −0.93833707 | 0.00015376 | Q00536 |
| 16 | 16 | 0 | CDK16 | Q00536-2 | wp137_DD.03.106 | DD.03.106 | down | −1.17537422 | 0.00295829 | Q00536 |
| 16 | 16 | 0 | CDK16 | Q00536-2 | wp146_TMX.01.160 | TMX.01.160 | down | −1.11003409 | 0.00026336 | Q00536 |
| 16 | 16 | 0 | CDK16 | Q00536-2 | wp146_TMX.02.138 | TMX.02.138 | down | −1.43368378 | 0.00010422 | Q00536 |
| 16 | 16 | 0 | CDK16 | Q00536-2 | wp146_TMX.02.174 | TMX.02.174 | down | −1.1760616 | 0.00021374 | Q00536 |
| 16 | 16 | 0 | CDK16 | Q00536-2 | wp147_DD.03.106_2 uM | DD.03.106 | down | −0.53851311 | 0.00149633 | Q00536 |
| 16 | 16 | 0 | CDK16 | Q00536-2 | wp147_DD.03.106_1 uM | DD.03.106 | down | −0.54267085 | 0.00144398 | Q00536 |
| 16 | 16 | 0 | CDK16 | Q00536-2 | wp147_DD.03.106_4 uM | DD.03.106 | down | −0.43926957 | 0.00375656 | Q00536 |
| 16 | 16 | 0 | CDK16 | Q00536-1 | wp172_SK.3.91 | SK.3.91 | down | −0.81688154 | 9.31E−05 | Q00536 |
| 16 | 16 | 0 | CDK16 | Q00536-1 | wp195_JWG.123 | JWG.123 | down | −0.5025985 | 0.00177219 | Q00536 |
| 16 | 12 | 4 | AK4 | P27144 | wp056_SB1.G.187 | SB1.G.187 | down | −0.44782967 | 0.00528565 | P27144 |
| 16 | 12 | 4 | AK4 | P27144 | wp071_JWG.118 | JWG.118 | down | −0.53138665 | 0.00721282 | P27144 |
| 16 | 12 | 4 | AK4 | P27144 | wp081_INY.01.140.1 | INY.01.140.1 | down | −0.43085656 | 0.0003895 | P27144 |
| 16 | 12 | 4 | AK4 | P27144 | wp084_BSJ.03.123 | BSJ.03.123 | down | −0.5102615 | 0.00077168 | P27144 |
| 16 | 12 | 4 | AK4 | P27144 | wp084_BSJ.04.132 | BSJ.04.132 | down | −0.65505339 | 0.00012871 | P27144 |
| 16 | 12 | 4 | AK4 | P27144 | wp137_DD.03.107 | DD.03.107 | down | −0.39422175 | 0.00026911 | P27144 |
| 16 | 12 | 4 | AK4 | P27144 | wp137_INY.02.164 | INY.02.164 | down | −0.56014139 | 4.16E−05 | P27144 |
| 16 | 12 | 4 | AK4 | P27144 | wp147_DD.03.106_4 uM | DD.03.106 | down | −0.56516649 | 6.37E−05 | P27144 |
| 16 | 12 | 4 | AK4 | P27144 | wp147_DD.03.107_1 uM | DD.03.107 | down | −0.39919743 | 0.00035477 | P27144 |
| 16 | 12 | 4 | AK4 | P27144 | wp147_DD.03.107_2 uM | DD.03.107 | down | −0.40448189 | 0.00033281 | P27144 |
| 16 | 12 | 4 | AK4 | P27144 | wp147_DD.03.107_4 uM | DD.03.107 | down | −0.8980326 | 6.12E−06 | P27144 |
| 16 | 12 | 4 | AK4 | P27144 | wp160_SK.3.91_1 hr | SK.3.91 | down | −0.82324323 | 0.00786775 | P27144 |
| 15 | 12 | 3 | COQ8A | Q8NI60-1 | wp056_SB1.G.187 | SB1.G.187 | down | −0.38847897 | 0.00177038 | Q8NI60 |
| 15 | 12 | 3 | COQ8A | Q8NI60-1 | wp056_ZNL.02.096 | ZNL.02.096 | down | −0.43170887 | 0.00116896 | Q8NI60 |
| 15 | 12 | 3 | COQ8A | Q8NI60-1 | wp084_BSJ.03.123 | BSJ.03.123 | down | −0.46111063 | 0.00311509 | Q8NI60 |
| 15 | 12 | 3 | COQ8A | Q8NI60-1 | wp084_BS.J.04.132 | BSJ.04.132 | down | −0.69387079 | 0.00070149 | Q8NI60 |
| 15 | 12 | 3 | COQ8A | Q8NI60-1 | wp118_ZNL.02.096 | ZNL.02.096 | down | −0.42300855 | 0.00414666 | Q8NI60 |
| 15 | 12 | 3 | COQ8A | Q8NI60-1 | wp137_TL12.186 | TL12.186 | down | −0.45637325 | 7.52E−05 | Q8NI60 |
| 15 | 12 | 3 | COQ8A | Q8NI60-1 | wp147_DD.03.106_1 uM | DD.03.106 | down | −0.35476099 | 0.00093639 | Q8NI60 |
| 15 | 12 | 3 | COQ8A | Q8NI60-1 | wp147_DD.03.106_4 uM | DD.03.106 | down | −0.81585208 | 1.53E−05 | Q8NI60 |
| 15 | 12 | 3 | COQ8A | Q8NI60-1 | wp147_DD.03.107_1 uM | DD.03.107 | down | −0.66779107 | 4.22E−05 | Q8NI60 |
| 15 | 12 | 3 | COQ8A | Q8NI60-1 | wp147_DD.03.107_2 uM | DD.03.107 | down | −0.62879433 | 5.71E−05 | Q8NI60 |
| 15 | 12 | 3 | COQ8A | Q8NI60-1 | wp147_DD.03.107_4 uM | DD.03.107 | down | −1.1209713 | 3.04E−06 | Q8NI60 |
| 15 | 12 | 3 | COQ8A | Q8NI60-1 | wp160_SK.3.91_1 hr | SK.3.91 | down | −0.79588274 | 0.00551986 | Q8NI60 |
| 15 | 15 | 0 | ULK3 | Q6PHR2-4 | wp107_SK.3.91 | SK.3.91 | down | −1.08434425 | 5.82E−06 | Q6PHR2 |
| 15 | 15 | 0 | ULK3 | Q6PHR2-1 | wp016_SK.3.91_1 uM | SK.3.91 | down | −1.03029955 | 0.00032772 | Q6PHR2 |
| 15 | 15 | 0 | ULK3 | Q6PHR2-1 | wp045_DB0614 | DB0614 | down | −0.94606372 | 0.00012461 | Q6PHR2 |
| 15 | 15 | 0 | ULK3 | Q6PHR2-1 | wp045_RSS0628 | RSS0628 | down | −1.08600213 | 6.87E−05 | Q6PHR2 |
| 15 | 15 | 0 | ULK3 | Q6PHR2-1 | wp103_SK.3.91 | SK.3.91 | down | −0.9650601 | 1.71E−05 | Q6PHR2 |
| 15 | 15 | 0 | ULK3 | Q6PHR2-1 | wp105_SK.3.91 | SK.3.91 | down | −0.83701695 | 4.67E−06 | Q6PHR2 |
| 15 | 15 | 0 | ULK3 | Q6PHR2-1 | wp117_DB0614 | DB0614 | down | −0.83065526 | 9.39E−06 | Q6PHR2 |
| 15 | 15 | 0 | ULK3 | Q6PHR2-1 | wp131_TL12.186 | TL12.186 | down | −1.07408884 | 3.58E−07 | Q6PHR2 |
| 15 | 15 | 0 | ULK3 | Q6PHR2-1 | wp133_SK.3.91_2 hr | SK.3.91 | down | −0.58170419 | 1.06E−05 | Q6PHR2 |
| 15 | 15 | 0 | ULK3 | Q6PHR2-1 | wp133_SK.3.91_4 hr | SK.3.91 | down | −1.06182719 | 2.00E−07 | Q6PHR2 |
| 15 | 15 | 0 | ULK3 | Q6PHR2-1 | wp137_TL12.186 | TL12.186 | down | −1.17420175 | 1.27E−06 | Q6PHR2 |
| 15 | 15 | 0 | ULK3 | Q6PHR2-1 | wp137_TL13.97 | TL13.97 | down | −0.37294269 | 0.00060798 | Q6PHR2 |
| 15 | 15 | 0 | ULK3 | Q6PHR2-1 | wp160_SK.3.91_2 hr | SK.3.91 | down | −0.50295071 | 0.00103284 | Q6PHR2 |
| 15 | 15 | 0 | ULK3 | Q6PHR2-1 | wp160_SK.3.91_4 hr | SK.3.91 | down | −0.96885871 | 7.73E−05 | Q6PHR2 |
| 15 | 15 | 0 | ULK3 | Q6PHR2-1 | wp172_SK.3.91 | SK.3.91 | down | −0.81507385 | 0.00015948 | Q6PHR2 |
| 15 | 15 | 0 | MAP3K11 | Q16584 | wp103_SK.3.91 | SK.3.91 | down | −0.78377325 | 0.00705697 | Q16584 |
| 15 | 15 | 0 | MAP3K11 | Q16584 | wp107_DB0646 | DB0646 | down | −0.50772061 | 0.00373066 | Q16584 |
| 15 | 15 | 0 | MAP3K11 | Q16584 | wp107_SK.3.91 | SK.3.91 | down | −2.18221231 | 4.43E−06 | Q16584 |
| 15 | 15 | 0 | MAP3K11 | Q16584 | wp114_SK.3.91 | SK.3.91 | down | −0.47737077 | 0.00999137 | Q16584 |
| 15 | 15 | 0 | MAP3K11 | Q16584 | wp117_DB0614 | DB0614 | down | −0.72913716 | 0.00012319 | Q16584 |
| 15 | 15 | 0 | MAP3K11 | Q16584 | wp117_DB0646 | DB0646 | down | −0.43083047 | 0.0016855 | Q16584 |
| 15 | 15 | 0 | MAP3K11 | Q16584 | wp131_TL12.186 | TL12.186 | down | −1.79497934 | 2.73E−06 | Q16584 |
| 15 | 15 | 0 | MAP3K11 | Q16584 | wp133_SK.3.91_1 hr | SK.3.91 | down | −0.39858917 | 0.00258124 | Q16584 |
| 15 | 15 | 0 | MAP3K11 | Q16584 | wp133_SK.3.91_2 hr | SK.3.91 | down | −0.84413352 | 5.78E−05 | Q16584 |
| 15 | 15 | 0 | MAP3K11 | Q16584 | wp133_SK.3.91_4 hr | SK.3.91 | down | −1.4866723 | 3.85E−06 | Q16584 |
| 15 | 15 | 0 | MAP3K11 | Q16584 | wp137_TL12.186 | TL12.186 | down | −1.49282596 | 2.39E−05 | Q16584 |
| 15 | 15 | 0 | MAP3K11 | Q16584 | wp137_TL13.97 | TL13.97 | down | −0.76258986 | 0.00082321 | Q16584 |
| 15 | 15 | 0 | MAP3K11 | Q16584 | wp146_TMX.02.138 | TMX.02.138 | down | −0.92413068 | 0.00487694 | Q16584 |
| 15 | 15 | 0 | MAP3K11 | Q16584 | wp146_TMX.02.174 | TMX.02.174 | down | −1.87373821 | 0.00040632 | Q16584 |
| 15 | 15 | 0 | MAP3K11 | Q16584 | wp168_DB0662 | DB0662 | down | −0.72881878 | 0.0044535 | Q16584 |
| 15 | 15 | 0 | ABL1 | P00519-2 | wp016_SK.3.91_1 uM | SK.3.91 | down | −0.38745468 | 0.00094978 | P00519 |
| 15 | 15 | 0 | ABL1 | P00519-2 | wp056_SB1.G.187 | SB1.G.187 | down | −0.5172976 | 0.00020825 | P00519 |
| 15 | 15 | 0 | ABL1 | P00519-2 | wp071_SB1.G.181 | SB1.G.181 | down | −1.73466481 | 0.00010021 | P00519 |
| 15 | 15 | 0 | ABL1 | P00519-2 | wp071_SB1.G.192.1 | SB1.G.192.1 | down | −1.41678272 | 0.00026112 | P00519 |
| 15 | 15 | 0 | ABL1 | P00519-2 | wp071_SB1.G.200 | SB1.G.200 | down | −1.64632277 | 0.0001285 | P00519 |
| 15 | 15 | 0 | ABL1 | P00519-2 | wp081_SB1.G.192.1 | SB1.G.192.1 | down | −1.18902116 | 9.30E−08 | P00519 |
| 15 | 15 | 0 | ABL1 | P00519-2 | wp103_SB1.G.187 | SB1.G.187 | down | −0.75400247 | 0.0008022 | P00519 |
| 15 | 15 | 0 | ABL1 | P00519-2 | wp105_SK.3.91 | SK.3.91 | down | −0.33298015 | 3.57E−05 | P00519 |
| 15 | 15 | 0 | ABL1 | P00519-2 | wp107_SB1.G.187 | SB1.G.187 | down | −0.53392935 | 2.38E−06 | P00519 |
| 15 | 15 | 0 | ABL1 | P00519-2 | wp107_SK.3.91 | SK.3.91 | down | −0.47106163 | 1.17E−05 | P00519 |
| 15 | 15 | 0 | ABL1 | P00519-2 | wp133_SK.3.91_4 hr | SK.3.91 | down | −0.3801103 | 7.66E−06 | P00519 |
| 15 | 15 | 0 | ABL1 | P00519 | wp152_DB1114 | DB1114 | down | −0.6212673 | 3.21E−07 | P00519 |
| 15 | 15 | 0 | ABL1 | P00519 | wp160_SK.3.91_4 hr | SK.3.91 | down | −0.35445913 | 0.00353098 | P00519 |
| 15 | 15 | 0 | ABL1 | P00519 | wp168_DB0662 | DB0662 | down | −0.76167251 | 2.66E−07 | P00519 |
| 15 | 15 | 0 | ABL1 | P00519 | wp168_DB1113 | DB1113 | down | −1.01427749 | 7.45E−08 | P00519 |
| 14 | 10 | 4 | AK3 | Q9UIJ7-1 | wp071_JWG.118 | JWG.118 | down | −0.42206204 | 0.00827653 | Q9UIJ7 |
| 14 | 10 | 4 | AK3 | Q9UIJ7-1 | wp076_MFH.5.103.1 | MFH.5.103.1 | down | −0.43101719 | 0.00378497 | Q9UIJ7 |
| 14 | 10 | 4 | AK3 | Q9UIJ7-1 | wp081_INY.01.140.1 | INY.01.140.1 | down | −0.41019108 | 3.78E−05 | Q9UIJ7 |
| 14 | 10 | 4 | AK3 | Q9UIJ7-1 | wp084_BSJ.03.123 | BSJ.03.123 | down | −0.45774534 | 0.00108116 | Q9UIJ7 |
| 14 | 10 | 4 | AK3 | Q9UIJ7-1 | wp084_BSJ.04.132 | BSJ.04.132 | down | −0.61241729 | 9.76E−05 | Q9UIJ7 |
| 14 | 10 | 4 | AK3 | Q9UIJ7-1 | wp147_DD.03.106_4 uM | DD.03.106 | down | −0.72020954 | 3.40E−05 | Q9UIJ7 |
| 14 | 10 | 4 | AK3 | Q9UIJ7-1 | wp147_DD.03.107_1 uM | DD.03.107 | down | −0.52652292 | 0.00016269 | Q9UIJ7 |
| 14 | 10 | 4 | AK3 | Q9UIJ7-1 | wp147_DD.03.107_2 uM | DD.03.107 | down | −0.56069683 | 0.00011917 | Q9UIJ7 |
| 14 | 10 | 4 | AK3 | Q9UIJ7-1 | wp147_DD.03.107_4 uM | DD.03.107 | down | −1.00057144 | 6.42E−06 | Q9UIJ7 |
| 14 | 10 | 4 | AK3 | Q9UIJ7-1 | wp160_SK.3.91_1 hr | SK.3.91 | down | −0.74341421 | 0.00327843 | Q9UIJ7 |
| 14 | 14 | 0 | MAP4K1 | Q92918 | wp016_SK.3.91_1 uM | SK.3.91 | down | −0.66771161 | 0.00010905 | Q92918 |
| 14 | 14 | 0 | MAP4K1 | Q92918 | wp045_DB0646 | DB0646 | down | −0.47886728 | 9.11E−05 | Q92918 |
| 14 | 14 | 0 | MAP4K1 | Q92918 | wp103_SK.3.91 | SK.3.91 | down | −0.50988082 | 4.35E−05 | Q92918 |
| 14 | 14 | 0 | MAP4K1 | Q92918 | wp107_SK.3.91 | SK.3.91 | down | −0.70358008 | 1.66E−06 | Q92918 |
| 14 | 14 | 0 | MAP4K1 | Q92918 | wp131_TL12.186 | TL12.186 | down | −0.45811786 | 3.25E−06 | Q92918 |
| 14 | 14 | 0 | MAP4K1 | Q92918 | wp133_SK.3.91_4 hr | SK.3.91 | down | −0.55283611 | 5.16E−07 | Q92918 |
| 14 | 14 | 0 | MAP4K1 | Q92918 | wp137_TL12.186 | TL12.186 | down | −0.61356656 | 1.43E−06 | Q92918 |
| 14 | 14 | 0 | MAP4K1 | Q92918 | wp152_DB0646 | DB0646 | down | −0.36381538 | 2.16E−05 | Q92918 |
| 14 | 14 | 0 | MAP4K1 | Q92918 | wp152_DB1114 | DB1114 | down | −0.66114084 | 8.32E−07 | Q92918 |
| 14 | 14 | 0 | MAP4K1 | Q92918 | wp160_DB0646_4 hr | DB0646 | down | −0.32280811 | 0.00020974 | Q92918 |
| 14 | 14 | 0 | MAP4K1 | Q92918 | wp160_DB0646_8 hr | DB0646 | down | −0.57851629 | 2.02E−05 | Q92918 |
| 14 | 14 | 0 | MAP4K1 | Q92918 | wp160_SK.3.91_4 hr | SK.3.91 | down | −0.4817458 | 4.23E−05 | Q92918 |
| 14 | 14 | 0 | MAP4K1 | Q92918 | wp168_DB0662 | DB0662 | down | −0.40672217 | 0.00299669 | Q92918 |
| 14 | 14 | 0 | MAP4K1 | Q92918 | wp168_DB1113 | DB1113 | down | −0.84630969 | 7.29E−05 | Q92918 |
| 14 | 14 | 0 | UHMK1 | Q8TAS1 | wp045_DB0646 | DB0646 | down | −0.39454221 | 0.00198787 | Q8TAS1 |
| 14 | 14 | 0 | UHMK1 | Q8TAS1 | wp076_MFH.5.115.2 | MFH.5.115.2 | down | −0.87741254 | 0.00073982 | Q8TAS1 |
| 14 | 14 | 0 | UHMK1 | Q8TAS1 | wp076_MFH.5.126.1 | MFH.5.126.1 | down | −0.47885384 | 0.00841626 | Q8TAS1 |
| 14 | 14 | 0 | UHMK1 | Q8TAS1 | wp107_DB0646 | DB0646 | down | −0.32627661 | 0.00019722 | Q8TAS1 |
| 14 | 14 | 0 | UHMK1 | Q8TAS1 | wp108_FMF.06.098.1 | FMF.06.098.1 | down | −0.38775618 | 0.00113287 | Q8TAS1 |
| 14 | 14 | 0 | UHMK1 | Q8TAS1 | wp117_DB0646 | DB0646 | down | −0.47219636 | 0.00049666 | Q8TAS1 |
| 14 | 14 | 0 | UHMK1 | Q8TAS1 | wp134_DB118430 | DB118430 | down | −0.49439189 | 0.0004035 | Q8TAS1 |
| 14 | 14 | 0 | UHMK1 | Q8TAS1 | wp160_DB0646_2 hr | DB0646 | down | −0.44678201 | 0.00174587 | Q8TAS1 |
| 14 | 14 | 0 | UHMK1 | Q8TAS1 | wp160_DB0646_4 hr | DB0646 | down | −0.75506777 | 0.00022456 | Q8TAS1 |
| 14 | 14 | 0 | UHMK1 | Q8TAS1 | wp160_DB0646_8 hr | DB0646 | down | −0.51957995 | 0.00097615 | Q8TAS1 |
| 14 | 14 | 0 | UHMK1 | Q8TAS1 | wp160_SK.3.91_2 hr | SK.3.91 | down | −0.414393 | 0.00232438 | Q8TAS1 |
| 14 | 14 | 0 | UHMK1 | Q8TAS1 | wp160_SK.3.91_4 hr | SK.3.91 | down | −0.39590066 | 0.00276128 | Q8TAS1 |
| 14 | 14 | 0 | UHMK1 | Q8TAS1 | wp168_DB0661 | DB0661 | down | −0.41602442 | 0.00036577 | Q8TAS1 |
| 14 | 14 | 0 | UHMK1 | Q8TAS1 | wp168_DB0662 | DB0662 | down | −0.39489636 | 0.00041504 | Q8TAS1 |
| 14 | 14 | 0 | CSNK1D | P48730-2 | wp056_WH._10417.099 | WH.10417.099 | down | −1.45541113 | 0.00021352 | P48730 |
| 14 | 14 | 0 | CSNK1D | P48730-2 | wp076_MFH.5.126.1 | MFH.5.126.1 | down | −0.92007881 | 0.00900936 | P48730 |
| 14 | 14 | 0 | CSNK1D | P48730-2 | wp103_WH.10417.099 | WH.10417.099 | down | −1.30068241 | 1.26E−05 | P48730 |
| 14 | 14 | 0 | CSNK1D | P48730-2 | wp107_WH.10417.099 | WH.10417.099 | down | −1.24849352 | 3.03E−05 | P48730 |
| 14 | 14 | 0 | CSNK1D | P48730-2 | wp108_FMF.08.098.1 | FMF.06.098.1 | down | −0.38124569 | 0.00666882 | P48730 |
| 14 | 14 | 0 | CSNK1D | P48730-2 | wp114_FMF.06.098.1 | FMF.06.098.1 | down | −0.4039454 | 0.00720969 | P48730 |
| 14 | 14 | 0 | CSNK1D | P48730-2 | wp114_SK.3.91 | SK.3.91 | down | −0.39908574 | 0.00752984 | P48730 |
| 14 | 14 | 0 | CSNK1D | P48730-2 | wp114_WH.10414.099 | WH.10414.099 | down | −0.61873551 | 0.00146341 | P48730 |
| 14 | 14 | 0 | CSNK1D | P48730-2 | wp137_TL12.186 | TL12.186 | down | −0.47211173 | 0.00021656 | P48730 |
| 14 | 14 | 0 | CSNK1D | P48730-2 | wp160_SK.3.91_4 hr | SK.3.91 | down | −0.33729819 | 0.00103854 | P48730 |
| 14 | 14 | 0 | CSNK1D | P48730-2 | wp172_WH.10417.099 | WH.10417.099 | down | −0.64008522 | 0.00436396 | P48730 |
| 14 | 14 | 0 | CSNK1D | P48730-1 | wp017_WH.9533.153 | WH.9533.153 | down | −0.77883434 | 2.63E−05 | P48730 |
| 14 | 14 | 0 | CSNK1D | P48730-1 | wp131_TL12.186 | TL12.186 | down | −0.35508974 | 0.00424498 | P48730 |
| 14 | 14 | 0 | CSNK1D | P48730-1 | wp133_SK.3.91_4 hr | SK.3.91 | down | −0.45852584 | 0.0002037 | P48730 |
| 14 | 13 | 1 | PLK4 | O00444 | wp103_SK.3.91 | SK.3.91 | down | −1.24298561 | 0.00053994 | O00444 |
| 14 | 13 | 1 | PLK4 | O00444 | wp107_SK.3.91 | SK.3.91 | down | −1.03562305 | 0.00313335 | O00444 |
| 14 | 13 | 1 | PLK4 | O00444 | wp117_DB0614 | DB0614 | down | −0.54144376 | 0.00373454 | O00444 |
| 14 | 13 | 1 | PLK4 | O00444 | wp131_FMF.06.098.1 | FMF.06.098.1 | down | −0.74346669 | 0.0003094 | O00444 |
| 14 | 13 | 1 | PLK4 | O00444 | wp131_TL12.186 | TL12.186 | down | −1.55864407 | 7.83E−05 | O00444 |
| 14 | 13 | 1 | PLK4 | O00444 | wp133_SK.3.91_1 hr | SK.3.91 | down | −0.72597458 | 0.00073332 | O00444 |
| 14 | 13 | 1 | PLK4 | O00444 | wp133_SK.3.91_2 hr | SK.3.91 | down | −1.52268669 | 1.28E−05 | O00444 |
| 14 | 13 | 1 | PLK4 | O00444 | wp133_SK.3.91_4 hr | SK.3.91 | down | −2.25840897 | 6.80E−07 | O00444 |
| 14 | 13 | 1 | PLK4 | O00444 | wp137_TL12.186 | TL12.186 | down | −1.15875687 | 0.00073328 | O00444 |
| 14 | 13 | 1 | PLK4 | O00444 | wp137_TL13.97 | TL13.97 | down | −1.04852883 | 0.00120838 | O00444 |
| 14 | 13 | 1 | PLK4 | O00444 | wp160_SK.3.91_2 hr | SK.3.91 | down | −0.9266222 | 0.00121143 | O00444 |
| 14 | 13 | 1 | PLK4 | O00444 | wp160_SK.3.91_4 hr | SK.3.91 | down | −1.16880154 | 0.00049003 | O00444 |
| 14 | 13 | 1 | PLK4 | O00444 | wp195_BJG.04.203 | BJG.04.203 | down | −0.37839221 | 0.00098543 | O00444 |
| 13 | 11 | 2 | TBRG4 | Q969Z0-1 | wp076_MFH.5.103.1 | MFH.5.103.1 | down | −0.38576252 | 0.00515674 | Q969Z0 |
| 13 | 11 | 2 | TBRG4 | Q969Z0-1 | wp081_INY.01.140.1 | INY.01.140.1 | down | −0.42076458 | 8.11E−05 | Q969Z0 |
| 13 | 11 | 2 | TBRG4 | Q969Z0-1 | wp081_SB1.G.192.1 | SB1.G.192.1 | down | −0.34524574 | 0.00022887 | Q969Z0 |
| 13 | 11 | 2 | TBRG4 | Q969Z0-1 | wp084_BSJ.03.123 | BS.J.03.123 | down | −0.49995717 | 0.00050075 | Q969Z0 |
| 13 | 11 | 2 | TBRG4 | Q969Z0-1 | wp084_BSJ.04.132 | BSJ.04.132 | down | −0.63953828 | 0.00010814 | Q969Z0 |
| 13 | 11 | 2 | TBRG4 | Q969Z0-1 | wp137_TL12.186 | TL12.186 | down | −0.34251539 | 7.43E−06 | Q969Z0 |
| 13 | 11 | 2 | TBRG4 | Q969Z0-1 | wp147_DD.03.106_4 uM | DD.03.106 | down | −0.75135746 | 4.28E−05 | Q969Z0 |
| 13 | 11 | 2 | TBRG4 | Q969Z0-1 | wp147_DD.03.107_1 uM | DD.03.107 | down | −0.62454791 | 0.00010783 | Q969ZO |
| 13 | 11 | 2 | TBRG4 | Q969Z0-1 | wp147_DD.03.107_2 uM | DD.03.107 | down | −0.62842453 | 0.00010457 | Q969Z0 |
| 13 | 11 | 2 | TBRG4 | Q969Z0-1 | wp147_DD.03.107_4 uM | DD.03.107 | down | −1.10505644 | 6.05E−06 | Q969Z0 |
| 13 | 11 | 2 | TBRG4 | Q969Z0-1 | wp160_SK.3.91_1 hr | SK.3.91 | down | −0.91125523 | 0.00592735 | Q969Z0 |
| 13 | 13 | 0 | STK35 | Q8TDR2 | wp056_WH.10417.099 | WH.10417.099 | down | −2.07260852 | 1.80E−05 | Q8TDR2 |
| 13 | 13 | 0 | STK35 | Q8TDR2 | wp103_WH.10417.099 | WH.10417.099 | down | −1.23873099 | 3.55E−05 | Q8TDR2 |
| 13 | 13 | 0 | STK35 | Q8TDR2 | wp107_WH.10417.099 | WH.10417.099 | down | −1.95372443 | 6.60E−08 | Q8TDR2 |
| 13 | 13 | 0 | STK35 | Q8TDR2 | wp108_FMF.06.098.1 | FMF.06.098.1 | down | −0.60289412 | 0.00023807 | Q8TDR2 |
| 13 | 13 | 0 | STK35 | Q8TDR2 | wp114_FMF.08.098.1 | FMF.06.098.1 | down | −0.51158409 | 0.0011286 | Q8TDR2 |
| 13 | 13 | 0 | STK35 | Q8TDR2 | wp114_WH.10414.099 | WH.10414.099 | down | −0.89020082 | 0.00012604 | Q8TDR2 |
| 13 | 13 | 0 | STK35 | Q8TDR2 | wp117_RSS0680 | RSS0680 | down | −0.39373931 | 0.00107268 | Q8TDR2 |
| 13 | 13 | 0 | STK35 | Q8TDR2 | wp131_FMF.06.098.1 | FMF.06.098.1 | down | −0.65604631 | 4.39E−06 | Q8TDR2 |
| 13 | 13 | 0 | STK35 | Q8TDR2 | wp131_TL12.186 | TL12.186 | down | −0.41349718 | 3.88E−05 | Q8TDR2 |
| 13 | 13 | 0 | STK35 | Q8TDR2 | wp137_TL12.186 | TL12.186 | down | −0.40789293 | 0.00086486 | Q8TDR2 |
| 13 | 13 | 0 | STK35 | Q8TDR2 | wp146_TMX.02.174 | TMX.02.174 | down | −0.56312884 | 0.00545746 | Q8TDR2 |
| 13 | 13 | 0 | STK35 | Q8TDR2 | wp152_DB1114 | DB1114 | down | −0.51342963 | 0.00300564 | Q8TDR2 |
| 13 | 13 | 0 | STK35 | Q8TDR2 | wp168_DB1113 | DB1113 | down | −0.76063347 | 1.74E−05 | Q8TDR2 |
| 13 | 9 | 4 | NADK2 | Q4GON4 | wp081_INY.01.140.1 | INY.01.140.1 | down | −0.38863682 | 0.00068307 | Q4GON4 |
| 13 | 9 | 4 | NADK2 | Q4GON4 | wp084_BSJ.03.123 | BSJ.03.123 | down | −0.48393592 | 0.00073107 | Q4GON4 |
| 13 | 9 | 4 | NADK2 | Q4GON4 | wp084_BSJ.04.132 | BSJ.04.132 | down | −0.5591496 | 0.00022523 | Q4GON4 |
| 13 | 9 | 4 | NADK2 | Q4GON4 | wp118_ZNL.02.096 | ZNL.02.096 | down | −0.40039227 | 0.00466149 | Q4GON4 |
| 13 | 9 | 4 | NADK2 | Q4GON4 | wp147_DD.03.108_4 uM | DD.03.106 | down | −0.69757909 | 0.00018286 | Q4GON4 |
| 13 | 9 | 4 | NADK2 | Q4GON4 | wp147_DD.03.107_1 uM | DD.03.107 | down | −0.54035731 | 0.00063219 | Q4GON4 |
| 13 | 9 | 4 | NADK2 | Q4GON4 | wp147_DD.03.107_2 uM | DD.03.107 | down | −0.44424643 | 0.0015873 | Q4GON4 |
| 13 | 9 | 4 | NADK2 | Q4GON4 | wp147_DD.03.107_4 uM | DD.03.107 | down | −0.95042027 | 3.91E−05 | Q4GON4 |
| 13 | 9 | 4 | NADK2 | Q4GON4 | wp160_SK.3.91_1 hr | SK.3.91 | down | −0.69800553 | 0.00484023 | Q4GON4 |
| 13 | 12 | 1 | FASTKD3 | Q14CZ7 | wp056_ZNL.02.096 | ZNL.02.096 | down | −0.71307444 | 0.00284577 | Q14CZ7 |
| 13 | 12 | 1 | FASTKD3 | Q14CZ7 | wp071_JWG.118 | JWG.118 | down | −0.43860964 | 0.00620365 | Q14CZ7 |
| 13 | 12 | 1 | FASTKD3 | Q14CZ7 | wp081_INY.01.140.1 | INY.01.140.1 | down | −0.38462942 | 0.00296248 | Q14CZ7 |
| 13 | 12 | 1 | FASTKD3 | Q14CZ7 | wp084_BSJ.03.123 | BSJ.03.123 | down | −0.4727982 | 0.00092937 | Q14CZ7 |
| 13 | 12 | 1 | FASTKD3 | Q14CZ7 | wp084_BSJ.04.132 | BSJ.04.132 | down | −0.54278976 | 0.0005376 | Q14CZ7 |
| 13 | 12 | 1 | FASTKD3 | Q14CZ7 | wp137_TL12.186 | TL12.186 | down | −0.62437528 | 0.00019682 | Q14CZ7 |
| 13 | 12 | 1 | FASTKD3 | Q14CZ7 | wp147_DD.03.106_4 uM | DD.03.106 | down | −0.82589238 | 0.00013689 | Q14CZ7 |
| 13 | 12 | 1 | FASTKD3 | Q14CZ7 | wp147_DD.03.107_1 uM | DD.03.107 | down | −0.69392823 | 0.00032187 | Q14CZ7 |
| 13 | 12 | 1 | FASTKD3 | Q14CZ7 | wp147_DD.03.107_2 uM | DD.03.107 | down | −0.69306819 | 0.00032382 | Q14CZ7 |
| 13 | 12 | 1 | FASTKD3 | Q14CZ7 | wp147_DD.03.107_4 uM | DD.03.107 | down | −1.1913746 | 2.18E−05 | Q14CZ7 |
| 13 | 12 | 1 | FASTKD3 | Q14CZ7 | wp152_DB0646 | DB0646 | down | −0.34184968 | 0.00142643 | Q14CZ7 |
| 13 | 12 | 1 | FASTKD3 | Q14CZ7 | wp160_SK.3.91_1 hr | SK.3.91 | down | −0.99699885 | 0.00205294 | Q14CZ7 |
| 13 | 12 | 1 | MAP3K1 | Q13233 | wp056_SB1.G.187 | SB1.G.187 | down | −0.78088692 | 0.00040578 | Q13233 |
| 13 | 12 | 1 | MAP3K1 | Q13233 | wp056_WH.10417.099 | WH.10417.099 | down | −0.50337417 | 0.00231897 | Q13233 |
| 13 | 12 | 1 | MAP3K1 | Q13233 | wp076_MFH.5.115.2 | MFH.5.115.2 | down | −0.3527862 | 0.0068509 | Q13233 |
| 13 | 12 | 1 | MAP3K1 | Q13233 | wp076_MFH.6.126.1 | MFH.5.126.1 | down | −0.5946809 | 0.00083587 | Q13233 |
| 13 | 12 | 1 | MAP3K1 | Q13233 | wp103_SB1.G.187 | SB1.G.187 | down | −0.58154844 | 0.00544403 | Q13233 |
| 13 | 12 | 1 | MAP3K1 | Q13233 | wp105_SK.3.91 | SK.3.91 | down | −0.32576205 | 2.69E−05 | Q13233 |
| 13 | 12 | 1 | MAP3K1 | Q13233 | wp107_DB0646 | DB0646 | down | −0.37667796 | 0.00534669 | Q13233 |
| 13 | 12 | 1 | MAP3K1 | Q13233 | wp107_SB1.G.187 | SB1.G.187 | down | −0.66928383 | 0.00057506 | Q13233 |
| 13 | 12 | 1 | MAP3K1 | Q13233 | wp107_WH.10417.099 | WH.10417.099 | down | −0.46734869 | 0.00262043 | Q13233 |
| 13 | 12 | 1 | MAP3K1 | Q13233 | wp152_DB0646 | DB0646 | down | −0.61385951 | 3.79E−05 | Q13233 |
| 13 | 12 | 1 | MAP3K1 | Q13233 | wp160_DB0646_8 hr | DB0646 | down | −0.72407688 | 0.00552342 | Q13233 |
| 13 | 12 | 1 | MAP3K1 | Q13233 | wp168_DB0662 | DB0662 | down | −0.70867761 | 1.62E−05 | Q13233 |
| 13 | 13 | 0 | BUB1B | O60566-3 | wp016_SK.3.91_1 uM | SK.3.91 | down | −0.4685152 | 0.00651015 | O60566 |
| 13 | 13 | 0 | BUB1B | O60566-3 | wp076_MFH.5.115.2 | MFH.5.115.2 | down | −0.50127116 | 0.00063503 | O60566 |
| 13 | 13 | 0 | BUB1B | O60566-3 | wp076_MFH.5.126.1 | MFH.5.126.1 | down | −0.7531844 | 0.00011103 | O60566 |
| 13 | 13 | 0 | BUB1B | O60566-3 | wp103_SK.3.91 | SK.3.91 | down | −0.39825771 | 0.00038212 | O60566 |
| 13 | 13 | 0 | BUB1B | O60566-3 | wp107_SK.3.91 | SK.3.91 | down | −0.5037114 | 9.42E−07 | O60566 |
| 13 | 13 | 0 | BUB1B | O60566-3 | wp108_FMF.06.098.1 | FMF.06.098.1 | down | −0.44260537 | 3.98E−05 | O60566 |
| 13 | 13 | 0 | BUB1B | O60566-3 | wp131_FMF.06.098.1 | FMF.06.098.1 | down | −0.42320089 | 3.11E−06 | O60566 |
| 13 | 13 | 0 | BUB1B | O60566-3 | wp131_TL12.186 | TL12.186 | down | −0.54101208 | 9.92E−07 | O60566 |
| 13 | 13 | 0 | BUB1B | O60566-3 | wp133_SK.3.91_1 hr | SK.3.91 | down | −0.40825453 | 2.69E−07 | O60566 |
| 13 | 13 | 0 | BUB1B | O60566-3 | wp133_SK.3.91_2 hr | SK.3.91 | down | −0.41953676 | 1.43E−07 | O60566 |
| 13 | 13 | 0 | BUB1B | O60566-3 | wp133_SK.3.91_4 hr | SK.3.91 | down | −0.58709806 | 4.60E−08 | O60566 |
| 13 | 13 | 0 | BUB1B | O60566-3 | wp137_TL12.186 | TL12.186 | down | −0.55156495 | 7.36E−07 | O60566 |
| 13 | 13 | 0 | BUB1B | O60566-3 | wp137_TL13.97 | TL13.97 | down | −0.33131958 | 1.22E−05 | O60566 |
| 12 | 12 | 0 | GSK3B | P49841-2 | wp017_WH.9533.153 | WH.9533.153 | down | −1.31665653 | 1.80E−06 | P49841 |
| 12 | 12 | 0 | GSK3B | P49841-2 | wp056_SB1.G.190 | SB1.G.190 | down | −0.63095586 | 0.00151959 | P49841 |
| 12 | 12 | 0 | GSK3B | P49841-2 | wp056_WH.10417.099 | WH.10417.099 | down | −1.29357767 | 8.35E−05 | P49841 |
| 12 | 12 | 0 | GSK3B | P49841-2 | wp068_FMF.04.147.1 | FMF.04.147.1 | down | −0.62184045 | 4.33E−05 | P49841 |
| 12 | 12 | 0 | GSK3B | P49841-2 | wp078_MFH.5.126.1 | MFH.5.126.1 | down | −0.96294962 | 0.00014627 | P49841 |
| 12 | 12 | 0 | GSK3B | P49841-2 | wp103_WH.10417.099 | WH.10417.099 | down | −1.44224376 | 4.93E−05 | P49841 |
| 12 | 12 | 0 | GSK3B | P49841-2 | wp105_WH.10417.099 | WH.10417.099 | down | −0.84030726 | 2.07E−07 | P49841 |
| 12 | 12 | 0 | GSK3B | P49841-2 | wp107_WH.10417.099 | WH.10417.099 | down | −1.44567455 | 4.47E−08 | P49841 |
| 12 | 12 | 0 | GSK3B | P49841-2 | wp146_TMX.02.138 | TMX.02.138 | down | −0.60400236 | 0.00262253 | P49841 |
| 12 | 12 | 0 | GSK3B | P49841-2 | wp146_TMX.02.174 | TMX.02.174 | down | −0.64720352 | 0.00206159 | P49841 |
| 12 | 12 | 0 | GSK3B | P49841-1 | wp172_WH.10417.099 | WH.10417.099 | down | −0.82979411 | 1.22E−05 | P49841 |
| 12 | 12 | 0 | GSK3B | P49841-1 | wp195_BJG.04.203 | BJG.04.203 | down | −0.5348456 | 6.74E−06 | P49841 |
| 12 | 12 | 0 | MAPKAPK2 | P49137 | wp045_DB0646 | DB0646 | down | −0.43344655 | 0.00017172 | P49137 |
| 12 | 12 | 0 | MAPKAPK2 | P49137 | wp105_DB0646 | DB0646 | down | −0.45620689 | 0.00107403 | P49137 |
| 12 | 12 | 0 | MAPKAPK2 | P49137 | wp107_DB0646 | DB0646 | down | −0.37341478 | 4.96E−06 | P49137 |
| 12 | 12 | 0 | MAPKAPK2 | P49137 | wp114_DB0646 | DB0646 | down | −0.49108062 | 0.00079089 | P49137 |
| 12 | 12 | 0 | MAPKAPK2 | P49137 | wp117_DB0646 | DB0646 | down | −0.43332789 | 6.84E−06 | P49137 |
| 12 | 12 | 0 | MAPKAPK2 | P49137 | wp152_DB0646 | DB0646 | down | −0.42232515 | 5.50E−06 | P49137 |
| 12 | 12 | 0 | MAPKAPK2 | P49137 | wp152_DB1114 | DB1114 | down | −2.07828597 | 8.40E−10 | P49137 |
| 12 | 12 | 0 | MAPKAPK2 | P49137 | wp160_DB0646_4 hr | DB0646 | down | −0.34435983 | 0.00014327 | P49137 |
| 12 | 12 | 0 | MAPKAPK2 | P49137 | wp160_DB0646_8 hr | DB0646 | down | −0.62274723 | 1.33E−05 | P49137 |
| 12 | 12 | 0 | MAPKAPK2 | P49137 | wp168_DB0662 | DB0662 | down | −0.33099875 | 0.00012312 | P49137 |
| 12 | 12 | 0 | MAPKAPK2 | P49137 | wp168_DB1113 | DB1113 | down | −3.2010019 | 1.24E−08 | P49137 |
| 12 | 12 | 0 | MAPKAPK2 | P49137 | wp172_DB0646 | DB0646 | down | −0.3293576 | 0.0008151 | P49137 |
| 12 | 12 | 0 | EPHA3 | P29320 | wp045_DB0646 | DB0646 | down | −0.53330369 | 0.00036732 | P29320 |
| 12 | 12 | 0 | EPHA3 | P29320 | wp071_SB1.G.192.1 | SB1.G.192.1 | down | −1.17535338 | 2.38E−05 | P29320 |
| 12 | 12 | 0 | EPHA3 | P29320 | wp081_SB1.G.192.1 | SB1.G.192.1 | down | −1.02698873 | 4.28E−06 | P29320 |
| 12 | 12 | 0 | EPHA3 | P29320 | wp103_DB0646 | DB0646 | down | −0.37150959 | 0.00181373 | P29320 |
| 12 | 12 | 0 | EPHA3 | P29320 | wp107_DB0646 | DB0646 | down | −0.43461205 | 3.14E−05 | P29320 |
| 12 | 12 | 0 | EPHA3 | P29320 | wp117_DB0646 | DB0646 | down | −0.35867998 | 0.00155242 | P29320 |
| 12 | 12 | 0 | EPHA3 | P29320 | wp131_SB1.G.192.1 | SB1.G.192.1 | down | −1.31789656 | 2.51E−07 | P29320 |
| 12 | 12 | 0 | EPHA3 | P29320 | wp152_DB0646 | DB0646 | down | −0.38709114 | 6.66E−05 | P29320 |
| 12 | 12 | 0 | EPHA3 | P29320 | wp152_DB1114 | DB1114 | down | −0.51585742 | 1.42E−05 | P29320 |
| 12 | 12 | 0 | EPHA3 | P29320 | wp160_DB0646_4 hr | DB0646 | down | −0.58136166 | 0.00011145 | P29320 |
| 12 | 12 | 0 | EPHA3 | P29320 | wp160_DB0646_8 hr | DB0646 | down | −0.55275627 | 0.00013634 | P29320 |
| 12 | 12 | 0 | EPHA3 | P29320 | wp168_DB1113 | DB1113 | down | −0.41821519 | 7.43E−06 | P29320 |
| 11 | 11 | 0 | PTK2 | Q05397-5 | wp012_TI13.112 | TI13.112 | down | −0.62171764 | 4.81E−07 | Q05397 |
| 11 | 11 | 0 | PTK2 | Q05397 | wp011_TL13.12 | TL13.12 | down | −1.3652233 | 3.12E−09 | Q05397 |
| 11 | 11 | 0 | PTK2 | Q05397 | wp105_DB0646 | DB0646 | down | −0.55754029 | 1.01E−06 | Q05397 |
| 11 | 11 | 0 | PTK2 | Q05397 | wp105_SK.3.91 | SK.3.91 | down | −2.9576033 | 1.91E−10 | Q05397 |
| 11 | 11 | 0 | PTK2 | Q05397 | wp114_DB0646 | DB0646 | down | −0.55384859 | 0.00019162 | Q05397 |
| 11 | 11 | 0 | PTK2 | Q05397 | wp114_FMF.06.098.1 | FMF.06.098.1 | down | −0.94774948 | 2.20E−05 | Q05397 |
| 11 | 11 | 0 | PTK2 | Q05397 | wp114_SK.3.91 | SK.3.91 | down | −1.6843475 | 2.13E−06 | Q05397 |
| 11 | 11 | 0 | PTK2 | Q05397 | wp146_TMX.02.172 | TMX.02.172 | down | −0.73810815 | 0.00115382 | Q05397 |
| 11 | 11 | 0 | PTK2 | Q05397 | wp146_TMX.02.174 | TMX.02.174 | down | −0.81978454 | 0.00079462 | Q05397 |
| 11 | 11 | 0 | PTK2 | Q05397 | wp172_DB0646 | DB0646 | down | −0.80471777 | 1.97E−06 | Q05397 |
| 11 | 11 | 0 | PTK2 | Q05397 | wp172_SK.3.91 | SK.3.91 | down | −2.6885916 | 8.60E−10 | Q05397 |
| 11 | 10 | 1 | GK | P32189-3 | wp056_SB1.G.187 | SB1.G.187 | down | −0.47191812 | 0.00104039 | P32189 |
| 11 | 10 | 1 | GK | P32189-3 | wp056_ZNL.02.096 | ZNL.02.096 | down | −0.38670149 | 0.00227333 | P32189 |
| 11 | 10 | 1 | GK | P32189-3 | wp071_JWG.118 | JWG.118 | down | −0.43168137 | 0.00377258 | P32189 |
| 11 | 10 | 1 | GK | P32189-3 | wp084_BSJ.03.123 | BSJ.03.123 | down | −0.41086364 | 0.00778995 | P32189 |
| 11 | 10 | 1 | GK | P32189-3 | wp084_BSJ.04.132 | BSJ.04.132 | down | −0.38297593 | 0.00822126 | P32189 |
| 11 | 10 | 1 | GK | P32189-3 | wp114_DB0646 | DB0646 | down | −0.36316996 | 0.00075973 | P32189 |
| 11 | 10 | 1 | GK | P32189-3 | wp147_DD.03.106_4 uM | DD.03.106 | down | −0.48114237 | 5.29E−05 | P32189 |
| 11 | 10 | 1 | GK | P32189-3 | wp147_DD.03.107_1 uM | DD.03.107 | down | −0.49791407 | 4.45E−05 | P32189 |
| 11 | 10 | 1 | GK | P32189-3 | wp147_DD.03.107_2 uM | DD.03.107 | down | −0.43243175 | 9.02E−05 | P32189 |
| 11 | 10 | 1 | GK | P32189-3 | wp147_DD.03.107_4 uM | DD.03.107 | down | −0.75915624 | 5.25E−06 | P32189 |
| 11 | 11 | 0 | CDK11B | P21127-3 | wp103_DB0646 | DB0646 | down | −0.3333512 | 0.00943752 | P21127 |
| 11 | 11 | 0 | CDK11B | P21127-1 | wp045_DB0646 | DB0646 | down | −0.45549472 | 0.00178658 | P21127 |
| 11 | 11 | 0 | CDK11B | P21127-1 | wp105_DB0646 | DB0646 | down | −0.35424773 | 0.00698255 | P21127 |
| 11 | 11 | 0 | CDK11B | P21127-1 | wp117_DB0646 | DB0646 | down | −0.46945905 | 0.00098408 | P21127 |
| 11 | 11 | 0 | CDK11B | P21127-1 | wp160_DB0646_4 hr | DB0646 | down | −0.51219438 | 0.00034046 | P21127 |
| 11 | 11 | 0 | CDK11B | P21127-1 | wp160_DB0646_8 hr | DB0646 | down | −0.64401232 | 0.00013705 | P21127 |
| 11 | 11 | 0 | CDK11B | P21127-1 | wp168_DB0662 | DB0662 | down | −0.59461495 | 1.21E−06 | P21127 |
| 11 | 11 | 0 | CDK11B | P21127-1 | wp168_DB1113 | DB1113 | down | −0.87357786 | 1.14E−07 | P21127 |
| 11 | 11 | 0 | CDK11B | P21127-1 | wp172_DB0646 | DB0646 | down | −0.46241561 | 0.00025053 | P21127 |
| 11 | 11 | 0 | CDK11B | P21127 | wp152_DB0646 | DB0646 | down | −0.59475445 | 2.67E−08 | P21127 |
| 11 | 11 | 0 | CDK11B | P21127 | wp152_DB1114 | DB1114 | down | −0.95929242 | 1.89E−09 | P21127 |
| 11 | 10 | 1 | CDK1 | P06493 | wp104_TMX.01.160.1_0.25 | TMX.01.160.1 | down | −0.79360771 | 3.43E−06 | P06493 |
| 11 | 10 | 1 | CDK1 | P06493 | wp104_TMX.01.160.1_1 | TMX.01.160.1 | down | −0.94129757 | 1.44E−06 | P06493 |
| 11 | 10 | 1 | CDK1 | P06493 | wp117_RSS0680 | RSS0680 | down | −0.35865716 | 2.73E−05 | P06493 |
| 11 | 10 | 1 | CDK1 | P06493 | wp131_FMF.06.098.1 | FMF.06.098.1 | down | −0.35352787 | 0.00016092 | P06493 |
| 11 | 10 | 1 | CDK1 | P06493 | wp146_TMX.01.160 | TMX.01.160 | down | −1.42273796 | 0.0004156 | P06493 |
| 11 | 10 | 1 | CDK1 | P06493 | wp146_TMX.02.138 | TMX.02.138 | down | −1.64124466 | 0.00024833 | P06493 |
| 11 | 10 | 1 | CDK1 | P06493 | wp146_TMX.02.172 | TMX.02.172 | down | −1.3024657 | 0.0005706 | P06493 |
| 11 | 10 | 1 | CDK1 | P06493 | wp146_TMX.02.174 | TMX.02.174 | down | −1.62901161 | 0.00025513 | P06493 |
| 11 | 10 | 1 | CDK1 | P06493 | wp147_DD.03.106_2 uM | DD.03.106 | down | −0.50151666 | 2.03E−06 | P06493 |
| 11 | 10 | 1 | CDK1 | P06493 | wp147_DD.03.106_4 uM | DD.03.106 | down | −0.54160526 | 1.37E−06 | P06493 |
| 10 | 10 | 0 | RIOK2 | Q9BVS4 | wp016_SK.3.91_1 uM | SK.3.91 | down | −0.63187464 | 5.10E−05 | Q9BVS4 |
| 10 | 10 | 0 | RIOK2 | Q9BVS4 | wp076_MFH.5.115.2 | MFH.5.115.2 | down | −0.80672569 | 9.63E−05 | Q9BVS4 |
| 10 | 10 | 0 | RIOK2 | Q9BVS4 | wp076_MFH.6.126.1 | MFH.5.126.1 | down | −0.78740412 | 0.000107 | Q9BVS4 |
| 10 | 10 | 0 | RIOK2 | Q9BVS4 | wp103_SK.3.91 | SK.3.91 | down | −0.83110379 | 0.00010885 | Q9BVS4 |
| 10 | 10 | 0 | RIOK2 | Q9BVS4 | wp105_SK.3.91 | SK.3.91 | down | −0.48203564 | 3.58E−05 | Q9BVS4 |
| 10 | 10 | 0 | RIOK2 | Q9BVS4 | wp107_SK.3.91 | SK.3.91 | down | −0.73932534 | 1.88E−06 | Q9BVS4 |
| 10 | 10 | 0 | RIOK2 | Q9BVS4 | wp131_TL12.186 | TL12.186 | down | −0.91342253 | 5.33E−08 | Q9BVS4 |
| 10 | 10 | 0 | RIOK2 | Q9BVS4 | wp133_SK.3.91_4 hr | SK.3.91 | down | −0.64759359 | 1.38E−07 | Q9BVS4 |
| 10 | 10 | 0 | RIOK2 | Q9BVS4 | wp137_TL12.186 | TL12.186 | down | −0.77275183 | 1.36E−07 | Q9BVS4 |
| 10 | 10 | 0 | RIOK2 | Q9BVS4 | wp160_SK.3.91_4 hr | SK.3.91 | down | −0.44164048 | 0.00016258 | Q9BVS4 |
| 10 | 1 | 9 | STK40 | Q8N2I9-4 | wp105_SK.3.91 | SK.3.91 | down | −0.6059938 | 0.00321919 | Q8N2I9 |
| 10 | 10 | 0 | MAP4K3 | Q8IVH8 | wp105_SK.3.91 | SK.3.91 | down | −0.57101942 | 2.31E−06 | Q8IVH8 |
| 10 | 10 | 0 | MAP4K3 | Q8IVH8 | wp107_SK.3.91 | SK.3.91 | down | −0.84561575 | 0.00017735 | Q8IVH8 |
| 10 | 10 | 0 | MAP4K3 | Q8IVH8 | wp108_FMF.06.098.1 | FMF.06.098.1 | down | −0.81401084 | 0.00075452 | Q8IVH8 |
| 10 | 10 | 0 | MAP4K3 | Q8IVH8 | wp114_FMF.06.098.1 | FMF.06.098.1 | down | −0.41461294 | 0.00317023 | Q8IVH8 |
| 10 | 10 | 0 | MAP4K3 | Q8IVH8 | wp131_FMF.06.098.1 | FMF.06.098.1 | down | −0.78979961 | 1.04E−05 | Q8IVH8 |
| 10 | 10 | 0 | MAP4K3 | Q8IVH8 | wp131_TL12.186 | TL12.186 | down | −0.62749354 | 8.87E−05 | Q8IVH8 |
| 10 | 10 | 0 | MAP4K3 | Q8IVH8 | wp133_SK.3.91_4 hr | SK.3.91 | down | −0.63212259 | 0.00021995 | Q8IVH8 |
| 10 | 10 | 0 | MAP4K3 | Q8IVH8 | wp137_TL12.186 | TL12.186 | down | −0.39969817 | 0.00221962 | Q8IVH8 |
| 10 | 10 | 0 | MAP4K3 | Q8IVH8 | wp168_DB1113 | DB1113 | down | −1.38507165 | 3.11E−06 | Q8IVH8 |
| 10 | 10 | 0 | MAP4K3 | Q8IVH8 | wp172_SK.3.91 | SK.3.91 | down | −0.79619196 | 0.0018385 | Q8IVH8 |
| 10 | 5 | 5 | PRAG1 | Q86YV5 | wp107_SK.3.91 | SK.3.91 | down | −0.36919538 | 0.005319 | Q86YV5 |
| 10 | 5 | 5 | PRAG1 | Q86YV5 | wp131_TL12.166 | TL12.186 | down | −0.42425221 | 3.70E−05 | Q86YV5 |
| 10 | 5 | 5 | PRAG1 | Q86YV5 | wp133_SK.3.91_2 hr | SK.3.91 | down | −0.36673132 | 0.00053045 | Q86YV5 |
| 10 | 5 | 5 | PRAG1 | Q86YV5 | wp133_SK.3.91_4 hr | SK.3.91 | down | −0.43516912 | 0.00167227 | Q86YV5 |
| 10 | 5 | 5 | PRAG1 | Q86YV5 | wp137_TL12.186 | TL12.186 | down | −0.34591883 | 0.00513466 | Q86YV5 |
| 10 | 10 | 0 | GSK3A | P49840 | wp017_WH.9533.153 | WH.9533.153 | down | −0.48332686 | 0.00980371 | P49840 |
| 10 | 10 | 0 | GSK3A | P49840 | wp056_SB1.G.190 | SB1.G.190 | down | −0.8893891 | 0.0001041 | P49840 |
| 10 | 10 | 0 | GSK3A | P49840 | wp056_WH.10417.099 | WH.10417.099 | down | −0.51685744 | 0.00094025 | P49840 |
| 10 | 10 | 0 | GSK3A | P49840 | wp068_FMF.04.147.1 | FMF.04.147.1 | down | −0.53460199 | 0.0006322 | P49840 |
| 10 | 10 | 0 | GSK3A | P49840 | wp076_MFH.5.126.1 | MFH.5.126.1 | down | −0.75310312 | 0.00052037 | P49840 |
| 10 | 10 | 0 | GSK3A | P49840 | wp103_WH.10417.099 | WH.10417.099 | down | −0.44297172 | 0.00091027 | P49840 |
| 10 | 10 | 0 | GSK3A | P49840 | wp105_WH.10417.099 | WH.10417.099 | down | −0.44990421 | 1.74E−05 | P49840 |
| 10 | 10 | 0 | GSK3A | P49840 | wp107_WH.10417.099 | WH.10417.099 | down | −0.46969422 | 0.0099399 | P49840 |
| 10 | 10 | 0 | GSK3A | P49840 | wp146_TMX.02.138 | TMX.02.138 | down | −0.8224599 | 0.00058738 | P49840 |
| 10 | 10 | 0 | GSK3A | P49840 | wp146_TMX.02.174 | TMX.02.174 | down | −0.71334143 | 0.00097596 | P49840 |
| 10 | 7 | 3 | TOP1 | P11387 | wp056_ZNL.02.096 | ZNL.02.096 | down | −0.37564109 | 0.00015176 | P11387 |
| 10 | 7 | 3 | TOP1 | P11387 | wp080_RSS0680 | RSS0680 | down | −0.4686858 | 7.70E−05 | P11387 |
| 10 | 7 | 3 | TOP1 | P11387 | wp084_BSJ.03.123 | BSJ.03.123 | down | −0.33814017 | 0.00021571 | P11387 |
| 10 | 7 | 3 | TOP1 | P11387 | wp147_DD.03.106_4 uM | DD.03.106 | down | −0.45250962 | 0.00012723 | P11387 |
| 10 | 7 | 3 | TOP1 | P11387 | wp147_DD.03.107_1 uM | DD.03.107 | down | −0.38228908 | 0.00029165 | P11387 |
| 10 | 7 | 3 | TOP1 | P11387 | wp147_DD.03.107_2 uM | DD.03.107 | down | −0.42341543 | 0.0001767 | P11387 |
| 10 | 7 | 3 | TOP1 | P11387 | wp147_DD.03.107_4 uM | DD.03.107 | down | −0.46988624 | 0.00010552 | P11387 |
| 10 | 10 | 0 | PAK4 | O96013-1 | wp056_ZNL.02.096 | ZNL.02.096 | down | −0.63396588 | 0.00587586 | O96013 |
| 10 | 10 | 0 | PAK4 | O96013-1 | wp081_ZNL.02.096 | ZNL.02.096 | down | −0.39887664 | 0.00027234 | O96013 |
| 10 | 10 | 0 | PAK4 | O96013-1 | wp103_WH.10417.099 | WH.10417.099 | down | −0.37046739 | 0.0026609 | O96013 |
| 10 | 10 | 0 | PAK4 | O96013-1 | wp105_WH.10417.099 | WH.10417.099 | down | −0.34778566 | 0.00012783 | O96013 |
| 10 | 10 | 0 | PAK4 | O96013-1 | wp106_ZNL.02.096_2 hr | ZNL.02.096 | down | −0.42381236 | 2.09E−05 | O96013 |
| 10 | 10 | 0 | PAK4 | O96013-1 | wp106_ZNL.02.096_4 hr | ZNL.02.096 | down | −0.57681562 | 1.43E−05 | O96013 |
| 10 | 10 | 0 | PAK4 | O96013-1 | wp107_WH.10417.099 | WH.10417.099 | down | −0.4797283 | 0.0001463 | O96013 |
| 10 | 10 | 0 | PAK4 | O96013-1 | wp118_ZNL.02.096 | ZNL.02.096 | down | −0.57320096 | 4.67E−05 | O96013 |
| 10 | 10 | 0 | PAK4 | O96013-1 | wp168_DB0662 | DB0662 | down | −1.33981216 | 1.78E−05 | O96013 |
| 10 | 10 | 0 | PAK4 | O96013-1 | wp195_BJG.04.203 | BJG.04.203 | down | −0.74864854 | 3.10E−06 | O96013 |
| 9 | 9 | 0 | BMP2K | Q9NSY1 | wp016_LT2.49 | LT2.49 | down | −0.5079017 | 0.00366646 | Q9NSY1 |
| 9 | 9 | 0 | BMP2K | Q9NSY1 | wp016_SK.3.91 | SK.3.91 | down | −0.47056948 | 0.00492191 | Q9NSY1 |
| 9 | 9 | 0 | BMP2K | Q9NSY1 | wp016_SK.3.93 | SK.3.93 | down | −0.45753738 | 0.00548364 | Q9NSY1 |
| 9 | 9 | 0 | BMP2K | Q9NSY1 | wp045_DB0614 | DB0614 | down | −0.39093047 | 0.00733811 | Q9NSY1 |
| 9 | 9 | 0 | BMP2K | Q9NSY1 | wp056_SB1.G.190 | SB1.G.190 | down | −0.35076139 | 0.00214382 | Q9NSY1 |
| 9 | 9 | 0 | BMP2K | Q9NSY1 | wp071_JWG.118 | JWG.118 | down | −0.35281014 | 0.00252085 | Q9NSY1 |
| 9 | 9 | 0 | BMP2K | Q9NSY1 | wp105_SK.3.91 | SK.3.91 | down | −0.40431715 | 0.00323366 | Q9NSY1 |
| 9 | 9 | 0 | BMP2K | Q9NSY1 | wp134_BSJ.04.178 | BSJ.04.178 | down | −0.69290015 | 8.93E−07 | Q9NSY1 |
| 9 | 9 | 0 | BMP2K | Q9NSY1 | wp195_BJG.04.203 | BJG.04.203 | down | −0.46192889 | 5.46E−05 | Q9NSY1 |
| 9 | 8 | 1 | MARK2 | Q7KZI7-14 | wp105_SK.3.91 | SK.3.91 | down | −0.41586162 | 0.00416989 | Q7KZI7 |
| 9 | 8 | 1 | MARK2 | Q7KZI7-14 | wp131_TL12.186 | TL12.186 | down | −0.48675544 | 1.63E−06 | Q7KZI7 |
| 9 | 8 | 1 | MARK2 | Q7KZI7-14 | wp133_SK.3.91_4 hr | SK.3.91 | down | −0.40860878 | 3.08E−06 | Q7KZI7 |
| 9 | 8 | 1 | MARK2 | Q7KZI7-1 | wp107_SK.3.91 | SK.3.91 | down | −0.52663474 | 0.00011566 | Q7KZI7 |
| 9 | 8 | 1 | MARK2 | Q7KZI7-1 | wp117_RSS0680 | RSS0680 | down | −0.42858328 | 0.0O149789 | Q7KZI7 |
| 9 | 8 | 1 | MARK2 | Q7KZI7-1 | wp134_BSJ.04.178 | BSJ.04.178 | down | −0.96856894 | 4.76E−05 | Q7KZI7 |
| 9 | 8 | 1 | MARK2 | Q7KZI7-1 | wp172_SK.3.91 | SK.3.91 | down | −0.63512822 | 4.23E−05 | Q7KZI7 |
| 9 | 8 | 1 | MARK2 | Q7KZI7-1 | wp195_BSJ.05.026 | BSJ.05.026 | down | −1.23858098 | 1.39E−08 | Q7KZI7 |
| 9 | 9 | 0 | CHKA | P35790 | wp076_MFH.5.126.1 | MFH.5.126.1 | down | −0.46530148 | 0.00596518 | P35790 |
| 9 | 9 | 0 | CHKA | P35790 | wp108_FMF.06.098.1 | FMF.06.098.1 | down | −0.37156887 | 0.00339159 | P35790 |
| 9 | 9 | 0 | CHKA | P35790 | wp131_FMF.06.098.1 | FMF.06.098.1 | down | −0.44572273 | 0.00013427 | P35790 |
| 9 | 9 | 0 | CHKA | P35790 | wp131_TL12.186 | TL12.186 | down | −0.39544851 | 0.00020785 | P35790 |
| 9 | 9 | 0 | CHKA | P35790 | wp152_DB0646 | DB0646 | down | −0.39786212 | 0.00231062 | P35790 |
| 9 | 9 | 0 | CHKA | P35790 | wp152_DB118430 | DB118430 | down | −0.36008461 | 0.00368528 | P35790 |
| 9 | 9 | 0 | CHKA | P35790 | wp160_DB0646_8 hr | DB0646 | down | −0.43029137 | 0.00211358 | P35790 |
| 9 | 9 | 0 | CHKA | P35790 | wp168_DB0661 | DB0661 | down | −0.36073374 | 0.00078887 | P35790 |
| 9 | 9 | 0 | CHKA | P35790 | wp168_DB0662 | DB0662 | down | −0.32222779 | 0.00082184 | P35790 |
| 9 | 9 | 0 | LATS1 | O95835 | wp071_SB1.G.181 | SB1.G.181 | down | −1.27333964 | 1.84E−05 | O95835 |
| 9 | 9 | 0 | LATS1 | O95835 | wp071_SB1.G.200 | SB1.G.200 | down | −1.40324608 | 1.15E−05 | O95835 |
| 9 | 9 | 0 | LATS1 | O95835 | wp105_SK.3.91 | SK.3.91 | down | −0.33349655 | 9.48E−05 | O95835 |
| 9 | 9 | 0 | LATS1 | O95835 | wp107_SK.3.91 | SK.3.91 | down | −0.46091298 | 1.01E−05 | O95835 |
| 9 | 9 | 0 | LATS1 | O95835 | wp117_RSS0680 | RSS0680 | down | −0.41425089 | 6.01E−05 | O95835 |
| 9 | 9 | 0 | LATS1 | O95835 | wp160_DB0646_8 hr | DB0646 | down | −0.33819426 | 0.00518011 | O95835 |
| 9 | 9 | 0 | LATS1 | O95835 | wp160_SK.3.91_4 hr | SK.3.91 | down | −0.34630571 | 0.00474945 | O95835 |
| 9 | 9 | 0 | LATS1 | O95835 | wp195_BJG.04.203 | BJG.04.203 | down | −0.39190694 | 0.00275191 | O95835 |
| 9 | 9 | 0 | LATS1 | O95835 | wp195_INY.04.125.01 | INY.04.125.01 | down | −0.41760383 | 0.00203188 | O95835 |
| 8 | 8 | 0 | NLK | Q9UBE8 | wp071_SB1.G.192.1 | SB1.G.192.1 | down | −0.55784169 | 0.0035209 | Q9UBE8 |
| 8 | 8 | 0 | NLK | Q9UBE8 | wp081_SB1.G.192.1 | SB1.G.192.1 | down | −0.48692005 | 0.00091274 | Q9UBE8 |
| 8 | 8 | 0 | NLK | Q9UBE8 | wp117_DB0646 | DB0646 | down | −0.50096303 | 0.00189654 | Q9UBE8 |
| 8 | 8 | 0 | NLK | Q9UBE8 | wp131_SB1.G.192.1 | SB1.G.192.1 | down | −0.56344683 | 0.00283154 | Q9UBE8 |
| 8 | 8 | 0 | NLK | Q9UBE8 | wp137_DD.03.106 | DD.03.108 | down | −0.4409959 | 0.00605402 | Q9UBE8 |
| 8 | 8 | 0 | NLK | Q9UBE8 | wp137_TL12.186 | TL12.186 | down | −0.5154126 | 0.00296344 | Q9UBE8 |
| 8 | 8 | 0 | NLK | Q9UBE8 | wp160_DB0646_8 hr | DB0646 | down | −0.55694297 | 0.0038041 | Q9UBE8 |
| 8 | 8 | 0 | NLK | Q9UBE8 | wp168_DB1113 | DB1113 | down | −0.85561582 | 3.52E−05 | Q9UBE8 |
| 8 | 8 | 0 | NEK9 | Q8TD19 | wp045_DB0614 | DB0614 | down | −2.7397916 | 2.96E−09 | Q8TD19 |
| 8 | 8 | 0 | NEK9 | Q8TD19 | wp045_RSS0628 | RSS0628 | down | −2.43765397 | 4.95E−09 | Q8TD19 |
| 8 | 8 | 0 | NEK9 | Q8TD19 | wp080_RSS0680 | RSS0680 | down | −1.27698055 | 7.14E−08 | Q8TD19 |
| 8 | 8 | 0 | NEK9 | Q8TD19 | wp117_DB0614 | DB0614 | down | −3.00640942 | 9.54E−08 | Q8TD19 |
| 8 | 8 | 0 | NEK9 | Q8TD19 | wp117_RSS0680 | RSS0680 | down | −1.48319203 | 3.04E−08 | Q8TD19 |
| 8 | 8 | 0 | NEK9 | Q8TD19 | wp146_TMX.02.174 | TMX.02.174 | down | −0.36255708 | 0.00662979 | Q8TD19 |
| 8 | 8 | 0 | NEK9 | Q8TD19 | wp195_BJG.04.203 | BJG.04.203 | down | −1.42496418 | 3.32E−09 | Q8TD19 |
| 8 | 8 | 0 | NEK9 | Q8TD19 | wp195_JWG.123 | JWG.123 | down | −1.07865454 | 1.58E−08 | Q8TD19 |
| 8 | 8 | 0 | PIP4K2C | Q8TBX8-1 | wp017_WH.9533.153 | WH.9533.153 | down | −0.71441732 | 0.00132913 | Q8TBX8 |
| 8 | 8 | 0 | PIP4K2C | Q8TBX8-1 | wp056_WH.10417.099 | WH.10417.099 | down | −0.50085152 | 0.00915888 | Q8TBX8 |
| 8 | 8 | 0 | PIP4K2C | Q8TBX8-1 | wp103_WH.10417.099 | WH.10417.099 | down | −0.52008219 | 8.88E−05 | Q8TBX8 |
| 8 | 8 | 0 | PIP4K2C | Q8TBX8-1 | wp105_WH.10417.099 | WH.10417.099 | down | −0.37147825 | 0.00089715 | Q8TBX8 |
| 8 | 8 | 0 | PIP4K2C | Q8TBX8-1 | wp107_DB0646 | DB0646 | down | −0.35301385 | 0.00293199 | Q8TBX8 |
| 8 | 8 | 0 | PIP4K2C | Q8TBX8-1 | wp107_WH.10417.099 | WH.10417.099 | down | −0.57341388 | 0.0001205 | Q8TBX8 |
| 8 | 8 | 0 | PIP4K2C | Q8TBX8-1 | wp117_DB0646 | DB0646 | down | −0.35028798 | 0.00053449 | Q8TBX8 |
| 8 | 8 | 0 | PIP4K2C | Q8TBX8-1 | wp160_DB0646_8 hr | DB0646 | down | −0.47138772 | 0.00070064 | Q8TBX8 |
| 8 | 8 | 0 | MAPK6 | Q16659 | wp080_RSS0680 | RSS0680 | down | −0.50331516 | 0.00545224 | Q16659 |
| 8 | 8 | 0 | MAPK6 | Q16659 | wp103_SK.3.91 | SK.3.91 | down | −1.52548703 | 0.00012059 | Q16659 |
| 8 | 8 | 0 | MAPK6 | Q16659 | wp107_SK.3.91 | SK.3.91 | down | −1.34498248 | 2.73E−05 | Q16659 |
| 8 | 8 | 0 | MAPK6 | Q16659 | wp118_INY.03.041.01 | INY.03.041.01 | down | −0.61832519 | 0.00406575 | Q16659 |
| 8 | 8 | 0 | MAPK6 | Q16659 | wp131_FMF.06.098.1 | FMF.06.098.1 | down | −0.41225707 | 0.00959752 | Q16659 |
| 8 | 8 | 0 | MAPK6 | Q16659 | wp131_TL12.186 | TL12.186 | down | −1.48800885 | 2.12E−05 | Q16659 |
| 8 | 8 | 0 | MAPK6 | Q16659 | wp133_SK.3.91_2 hr | SK.3.91 | down | −0.82527864 | 0.00011593 | Q16659 |
| 8 | 8 | 0 | MAPK6 | Q16659 | wp133_SK.3.91_4 hr | SK.3.91 | down | −1.61363385 | 3.18E−06 | Q16659 |
| 8 | 8 | 0 | PIP4K2B | P78356 | wp045_DB0646 | DB0646 | down | −1.29646041 | 3.10E−06 | P78356 |
| 8 | 8 | 0 | PIP4K2B | P78356 | wp103_DB0646 | DB0646 | down | −0.68475242 | 0.00032146 | P78356 |
| 8 | 8 | 0 | PIP4K2B | P78356 | wp107_DB0646 | DB0646 | down | −1.02390084 | 1.38E−05 | P78356 |
| 8 | 8 | 0 | PIP4K2B | P78356 | wp117_DB0646 | DB0646 | down | −1.02659569 | 3.17E−06 | P78356 |
| 8 | 8 | 0 | PIP4K2B | P78356 | wp152_DB0646 | DB0646 | down | −1.14691096 | 3.34E−07 | P78356 |
| 8 | 8 | 0 | PIP4K2B | P78356 | wp152_DB1114 | DB1114 | down | −0.80492887 | 2.34E−06 | P78356 |
| 8 | 8 | 0 | PIP4K2B | P78356 | wp160_DB0646_4 hr | DB0646 | down | −0.50245546 | 8.78E−06 | P78356 |
| 8 | 8 | 0 | PIP4K2B | P78356 | wp160_DB0646_8 hr | DB0646 | down | −0.88479114 | 8.88E−07 | P78356 |
| 8 | 8 | 0 | NEK2 | P51955 | wp103_SK_3.91 | SK.3.91 | down | −0.45643284 | 0.00315233 | P51955 |
| 8 | 8 | 0 | NEK2 | P51955 | wp105_SK.3.91 | SK.3.91 | down | −0.39075549 | 0.00616829 | P51955 |
| 8 | 8 | 0 | NEK2 | P51955 | wp107_SK.3.91 | SK.3.91 | down | −0.63684025 | 0.00024782 | P51955 |
| 8 | 8 | 0 | NEK2 | P51955 | wp131_TL12.186 | TL12.186 | down | −0.37829252 | 0.00517045 | P51955 |
| 8 | 8 | 0 | NEK2 | P51955 | wp133_SK.3.91_1 hr | SK.3.91 | down | −0.43967556 | 0.00066646 | P51955 |
| 8 | 8 | 0 | NEK2 | P51955 | wp133_SK.3.91_2 hr | SK.3.91 | down | −0.44113012 | 0.00134428 | P51955 |
| 8 | 8 | 0 | NEK2 | P51955 | wp133_SK.3.91_4 hr | SK.3.91 | down | −0.73029291 | 5.51E−05 | P51955 |
| 8 | 8 | 0 | NEK2 | P51955 | wp160_SK.3.91_4 hr | SK.3.91 | down | −0.42942459 | 0.00724775 | P51955 |
| 8 | 8 | 0 | RPS6KA3 | P51812 | wp076_MFH.6.103.1 | MFH.5.103.1 | down | −1.16544895 | 5.12E−06 | P51812 |
| 8 | 8 | 0 | RPS6KA3 | P51812 | wp076_MFH.5.116.1 | MFH.5.116.1 | down | −0.36901484 | 0.00075234 | P51812 |
| 8 | 8 | 0 | RPS6KA3 | P51812 | wp146_TMX.02.138 | TMX.02.138 | down | −0.40227478 | 0.00305006 | P51812 |
| 8 | 8 | 0 | RPS6KA3 | P51812 | wp146_TMX.02.172 | TMX.02.172 | down | −0.83485502 | 0.00022793 | P51812 |
| 8 | 8 | 0 | RPS6KA3 | P51812 | wp146_TMX.02.174 | TMX.02.174 | down | −0.43123215 | 0.00239669 | P51812 |
| 8 | 8 | 0 | RPS6KA3 | P51812 | wp160_DB0646_8 hr | DB0646 | down | −0.33315405 | 3.53E−05 | P51812 |
| 8 | 8 | 0 | RPS6KA3 | P51812 | wp168_DB0662 | DB0662 | down | −0.45197971 | 1.57E−06 | P51812 |
| 8 | 8 | 0 | RPS6KA3 | P51812 | wp168_DB1113 | DB1113 | down | −0.50788897 | 2.24E−06 | P51812 |
| 8 | 8 | 0 | CSNK1E | P49674 | wp056_WH.10417.099 | WH.10417.099 | down | −1.59128646 | 0.00229428 | P49674 |
| 8 | 8 | 0 | CSNK1E | P49674 | wp071_SB1.G.181 | SB1.G.181 | down | −1.18941688 | 0.00027288 | P49674 |
| 8 | 8 | 0 | CSNK1E | P49674 | wp071_SB1.G.200 | SB1.G.200 | down | −0.75997608 | 0.00210398 | P49674 |
| 8 | 8 | 0 | CSNK1E | P49674 | wp103_WH._10417.099 | WH.10417.099 | down | −1.30614418 | 2.73E−05 | P49674 |
| 8 | 8 | 0 | CSNK1E | P49674 | wp105_WH.10417.099 | WH.10417.099 | down | −0.83054115 | 6.93E−06 | P49674 |
| 8 | 8 | 0 | CSNK1E | P49674 | wp107_WH.10417.099 | WH.10417.099 | down | −2.19217902 | 2.18E−06 | P49674 |
| 8 | 8 | 0 | CSNK1E | P49674 | wp137_TL12.186 | TL12.186 | down | −0.44113753 | 0.00453494 | P49674 |
| 8 | 8 | 0 | CSNK1E | P49674 | wp172_WH.10417.099 | WH.10417.099 | down | −1.16380863 | 4.84E−07 | P49674 |
| 8 | 8 | 0 | TEC | P42680 | wp047_DD.03.119 | DD.03.119 | down | −2.27183951 | 3.21E−06 | P42680 |
| 8 | 8 | 0 | TEC | P42680 | wp103_SK.3.91 | SK.3.91 | down | −0.64967551 | 0.00224056 | P42680 |
| 8 | 8 | 0 | TEC | P42680 | wp107_DB0646 | DB0646 | down | −0.82820896 | 0.00068545 | P42680 |
| 8 | 8 | 0 | TEC | P42680 | wp107_SK.3.91 | SK.3.91 | down | −1.14569047 | 0.00034376 | P42680 |
| 8 | 8 | 0 | TEC | P42680 | wp134_DB118430 | DB118430 | down | −0.56663101 | 0.0016739 | P42680 |
| 8 | 8 | 0 | TEC | P42680 | wp137_TL12.186 | TL12.186 | down | −2.11032659 | 9.03E−05 | P42680 |
| 8 | 8 | 0 | TEC | P42680 | wp137_TL13.97 | TL13.97 | down | −1.7115582 | 0.00027243 | P42680 |
| 8 | 8 | 0 | TEC | P42680 | wp146_TMX.02.174 | TMX.02.174 | down | −0.93615299 | 0.005489 | P42680 |
| 8 | 8 | 0 | YES1 | P07947 | wp105_DB0646 | DB0646 | down | −0.39573073 | 0.00933445 | P07947 |
| 8 | 8 | 0 | YES1 | P07947 | wp131_SB1.G.192.1 | SB1.G.192.1 | down | −1.24381453 | 6.52E−05 | P07947 |
| 8 | 8 | 0 | YES1 | P07947 | wp131_TL12.186 | TL12.186 | down | −0.54373237 | 0.00226729 | P07947 |
| 8 | 8 | 0 | YES1 | P07947 | wp133_SK.3.91_4 hr | SK.3.91 | down | −0.50254138 | 0.00396364 | P07947 |
| 8 | 8 | 0 | YES1 | P07947 | wp168_DB0661 | DB0661 | down | −0.52291892 | 0.001846 | P07947 |
| 8 | 8 | 0 | YES1 | P07947 | wp168_DB0662 | DB0662 | down | −0.80245859 | 0.00020007 | P07947 |
| 8 | 8 | 0 | YES1 | P07947 | wp172_DB0646 | DB0646 | down | −0.47098434 | 0.00015388 | P07947 |
| 8 | 8 | 0 | YES1 | P07947 | wp172_SB1.G.187 | SB1.G.187 | down | −0.53244622 | 6.42E−05 | P07947 |
| 8 | 8 | 0 | STK10 | O94804 | wp016_LT2.49 | LT2.49 | down | −0.33362626 | 0.00079669 | O94804 |
| 8 | 8 | 0 | STK10 | O94804 | wp016_SK.3.89 | SK.3.89 | down | −0.3539335 | 0.0006232 | O94804 |
| 8 | 8 | 0 | STK10 | O94804 | wp016_TL12.186 | TL12.186 | down | −0.38193193 | 0.00045327 | O94804 |
| 8 | 8 | 0 | STK10 | O94804 | wp056_WH.10417.099 | WH.10417.099 | down | −0.4923436 | 6.21E−05 | O94804 |
| 8 | 8 | 0 | STK10 | O94804 | wp103_SB1.G.187 | SB1.G.187 | down | −0.33743872 | 0.00027533 | O94804 |
| 8 | 8 | 0 | STK10 | O94804 | wp103_WH.10417.099 | WH.10417.099 | down | −0.47506519 | 5.95E−05 | O94804 |
| 8 | 8 | 0 | STK10 | O94804 | wp107_WH.10417.099 | WH.10417.099 | down | −0.56611628 | 6.39E−07 | O94804 |
| 8 | 8 | 0 | STK10 | O94804 | wp137_TL13.97 | TL13.97 | down | −0.34773714 | 3.66E−05 | O94804 |
| 7 | 7 | 0 | MAP4K5 | Q9Y4K4 | wp045_DB0646 | DB0646 | down | −0.41085891 | 0.0020229 | Q9Y4K4 |
| 7 | 7 | 0 | MAP4K5 | Q9Y4K4 | wp105_SB1.G.187 | SB1.G.187 | down | −0.34876815 | 6.71E−06 | Q9Y4K4 |
| 7 | 7 | 0 | MAP4K5 | Q9Y4K4 | wp107_DB0646 | DB0646 | down | −0.33448481 | 8.66E−05 | Q9Y4K4 |
| 7 | 7 | 0 | MAP4K5 | Q9Y4K4 | wp107_SB1.G.187 | SB1.G.187 | down | −0.44114984 | 1.38E−05 | Q9Y4K4 |
| 7 | 7 | 0 | MAP4K5 | Q9Y4K4 | wp152_DB1114 | DB1114 | down | −0.85673634 | 3.22E−06 | Q9Y4K4 |
| 7 | 7 | 0 | MAP4K5 | Q9Y4K4 | wp160_DB0646_8 hr | DB0646 | down | −0.50090311 | 0.00013336 | Q9Y4K4 |
| 7 | 7 | 0 | MAP4K5 | Q9Y4K4 | wp168_DB1113 | DB1113 | down | −1.75425658 | 1.57E−08 | Q9Y4K4 |
| 7 | 7 | 0 | STK33 | Q9BYT3-1 | wp076_MFH.5.103.1 | MFH.5.103.1 | down | −2.78406575 | 0.00014517 | Q9BYT3 |
| 7 | 7 | 0 | STK33 | Q9BYT3-1 | wp076_MFH.5.116.1 | MFH.5.116.1 | down | −1.38178725 | 0.00276409 | Q9BYT3 |
| 7 | 7 | 0 | STK33 | Q9BYT3-1 | wp105_SK.3.91 | SK.3.91 | down | −2.05718078 | 2.58E−07 | Q9BYT3 |
| 7 | 7 | 0 | STK33 | Q9BYT3-1 | wp146_TMX.02.138 | TMX.02.138 | down | −1.33541766 | 0.00028973 | Q9BYT3 |
| 7 | 7 | 0 | STK33 | Q9BYT3-1 | wp146_TMX.02.172 | TMX.02.172 | down | −1.29518059 | 0.00032352 | Q9BYT3 |
| 7 | 7 | 0 | STK33 | Q9BYT3-1 | wp146_TMX.02.174 | TMX.02.174 | down | −1.76255783 | 0.00010604 | Q9BYT3 |
| 7 | 7 | 0 | STK33 | Q9BYT3-1 | wp172_SK.3.91 | SK.3.91 | down | −2.22697301 | 7.99E−05 | Q9BYT3 |
| 7 | 7 | 0 | ACAD10 | Q6JQN1-5 | wp084_BSJ.03.123 | BSJ.03.123 | down | −0.5274964 | 0.00118682 | Q6JQN1 |
| 7 | 7 | 0 | ACAD10 | Q6JQN1-5 | wp084_BSJ.04.132 | BSJ.04.132 | down | −0.74835637 | 0.00029225 | Q6JQN1 |
| 7 | 7 | 0 | ACAD10 | Q6JQN1-5 | wp147_DD.03.108_4 uM | DD.03.106 | down | −0.74044416 | 0.00104372 | Q6JQN1 |
| 7 | 7 | 0 | ACAD10 | Q6JQN1-5 | wp147_DD.03.107_1 uM | DD.03.107 | down | −0.55361632 | 0.00392207 | Q6JQN1 |
| 7 | 7 | 0 | ACAD10 | Q6JQN1-5 | wp147_DD.03.107_2 uM | DD.03.107 | down | −0.52340633 | 0.00500646 | Q6JQN1 |
| 7 | 7 | 0 | ACAD10 | Q6JQN1-5 | wp147_DD.03.107_4 uM | DD.03.107 | down | −1.06060483 | 0.00019334 | Q6JQN1 |
| 7 | 7 | 0 | ACAD10 | Q6JQN1 | wp056_SB1.G.187 | SB1.G.187 | down | −0.38379343 | 0.00763912 | Q6JQN1 |
| 7 | 6 | 1 | DGUOK | Q16854 | wp084_BSJ.03.123 | BSJ.03.123 | down | −0.43769327 | 0.00137764 | Q16854 |
| 7 | 6 | 1 | DGUOK | Q16854 | wp084_BSJ.04.132 | BSJ.04.132 | down | −0.52540121 | 0.00026821 | Q16854 |
| 7 | 6 | 1 | DGUOK | Q16854 | wp118_ZNL.02.096 | ZNL.02.096 | down | −0.53365011 | 0.00061391 | Q16854 |
| 7 | 6 | 1 | DGUOK | Q16854 | wp147_DD.03.106_4 uM | DD.03.106 | down | −0.54181105 | 0.00172975 | Q16854 |
| 7 | 6 | 1 | DGUOK | Q16854 | wp147_DD.03.107_1 uM | DD.03.107 | down | −0.52448069 | 0.00200742 | Q16854 |
| 7 | 6 | 1 | DGUOK | Q16854 | wp147_DD.03.107_4 uM | DD.03.107 | down | −0.96775665 | 0.00010601 | Q16854 |
| 7 | 7 | 0 | MAPK7 | Q13164-1 | wp016_SK.3.91_1 uM | SK.3.91 | down | −0.39238572 | 0.00791281 | Q13164 |
| 7 | 7 | 0 | MAPK7 | Q13164-1 | wp076_MFH.5.103.1 | MFH.5.103.1 | down | −0.41422399 | 0.00419879 | Q13164 |
| 7 | 7 | 0 | MAPK7 | Q13164-1 | wp108_FMF.06.098.1 | FMF.06.098.1 | down | −0.43466058 | 0.00040187 | Q13164 |
| 7 | 7 | 0 | MAPK7 | Q13164-1 | wp131_FMF.06.098.1 | FMF.06.098.1 | down | −0.46371858 | 5.93E−06 | Q13164 |
| 7 | 7 | 0 | MAPK7 | Q13164-1 | wp137_TL12.186 | TL12.186 | down | −0.33341941 | 0.00019633 | Q13164 |
| 7 | 7 | 0 | MAPK7 | Q13164-1 | wp168_DB1113 | DB1113 | down | −0.76601303 | 1.78E−06 | Q13164 |
| 7 | 7 | 0 | MAPK7 | Q13164 | wp152_DB1114 | DB1114 | down | −0.66167832 | 1.45E−06 | Q13164 |
| 7 | 7 | 0 | AKT2 | P31751 | wp104_INY.02.164 | INY.02.164 | down | −1.07758376 | 2.43E−07 | P31751 |
| 7 | 7 | 0 | AKT2 | P31751 | wp105_SK.3.91 | SK.3.91 | down | −0.87221344 | 1.05E−07 | P31751 |
| 7 | 7 | 0 | AKT2 | P31751 | wp107_SK.3.91 | SK.3.91 | down | −0.41410369 | 2.29E−05 | P31751 |
| 7 | 7 | 0 | AKT2 | P31751 | wp118_INY.03.041.01 | INY.03.041.01 | down | −2.77737877 | 1.02E−08 | P31751 |
| 7 | 7 | 0 | AKT2 | P31751 | wp135_INY.03.041_01_8 hr | INY.03.041_01 | down | −2.27384342 | 6.77E−10 | P31751 |
| 7 | 7 | 0 | AKT2 | P31751 | wp135_INY.03.041.01_4 hr | INY.03.041.01 | down | −1.73894503 | 8.00E−10 | P31751 |
| 7 | 7 | 0 | AKT2 | P31751 | wp137_INY.02.164 | INY.02.164 | down | −3.15594487 | 2.26E−10 | P31751 |
| 7 | 7 | 0 | BCKDK | O14874 | wp084_BSJ.03.123 | BSJ.03.123 | down | −0.45529596 | 0.00377679 | O14874 |
| 7 | 7 | 0 | BCKDK | O14874 | wp084_BSJ.04.132 | BSJ.04.132 | down | −0.51884245 | 0.00098577 | O14874 |
| 7 | 7 | 0 | BCKDK | O14874 | wp137_DD.03.106 | DD.03.106 | down | −0.32754126 | 0.0065767 | O14874 |
| 7 | 7 | 0 | BCKDK | O14874 | wp137_INY.02.164 | INY.02.164 | down | −0.36000005 | 0.00429856 | O14874 |
| 7 | 7 | 0 | BCKDK | O14874 | wp147_DD.03.106_4 uM | DD.03.106 | down | −0.58310295 | 0.00962168 | O14874 |
| 7 | 7 | 0 | BCKDK | O14874 | wp147_DD.03.107_4 uM | DD.03.107 | down | −1.1128554 | 0.0005271 | O14874 |
| 7 | 7 | 0 | BCKDK | O14874 | wp160_SK.3.91_1 hr | SK.3.91 | down | −0.86834193 | 0.00651874 | O14874 |
| 6 | 6 | 0 | AKT3 | Q9Y243-1 | wp104_INY.02.164 | INY.02.164 | down | −1.47841841 | 6.29E−07 | Q9Y243 |
| 6 | 6 | 0 | AKT3 | Q9Y243-1 | wp105_SK.3.91 | SK.3.91 | down | −0.77826619 | 8.85E−06 | Q9Y243 |
| 6 | 6 | 0 | AKT3 | Q9Y243-1 | wp118_INY.03.041.01 | INY.03.041.01 | down | −2.76869821 | 6.18E−07 | Q9Y243 |
| 6 | 6 | 0 | AKT3 | Q9Y243-1 | wp135_INY.03.041_01_8 hr | INY.03.041_01 | down | −2.21528297 | 1.04E−07 | Q9Y243 |
| 6 | 6 | 0 | AKT3 | Q9Y243-1 | wp135_INY.03.041.01_4 hr | INY.03.041.01 | down | −1.92468183 | 1.85E−07 | Q9Y243 |
| 6 | 6 | 0 | AKT3 | Q9Y243-1 | wp137_INY.02.164 | INY.02.164 | down | −3.14690657 | 1.34E−07 | Q9Y243 |
| 6 | 6 | 0 | IPPK | Q9H8X2 | wp103_SK.3.91 | SK.3.91 | down | −0.47290379 | 0.00840762 | Q9H8X2 |
| 6 | 6 | 0 | IPPK | Q9H8X2 | wp107_SK.3.91 | SK.3.91 | down | −1.00066622 | 0.00066226 | Q9H8X2 |
| 6 | 6 | 0 | IPPK | Q9H8X2 | wp114_FMF.06.098.1 | FMF.06.098.1 | down | −0.57731451 | 0.00655065 | Q9H8X2 |
| 6 | 6 | 0 | IPPK | Q9H8X2 | wp114_SK.3.91 | SK.3.91 | down | −0.78814249 | 0.00205505 | Q9H8X2 |
| 6 | 6 | 0 | IPPK | Q9H8X2 | wp131_TL12.186 | TL12.186 | down | −0.63106542 | 8.89E−05 | Q9H8X2 |
| 6 | 6 | 0 | IPPK | Q9H8X2 | wp133_SK.3.91_4 hr | SK.3.91 | down | −0.48163598 | 0.00068507 | Q9H8X2 |
| 6 | 6 | 0 | IRAK1 | P51617 | wp056_SB1.G.187 | SB1.G.187 | down | −1.5784794 | 2.41E−05 | P51617 |
| 6 | 6 | 0 | IRAK1 | P51617 | wp081_TL13.150 | TL13.150 | down | −2.83447585 | 1.36E−08 | P51617 |
| 6 | 6 | 0 | IRAK1 | P51617 | wp103_SB1.G.187 | SB1.G.187 | down | −1.50429323 | 0.0001169 | P51617 |
| 6 | 6 | 0 | IRAK1 | P51617 | wp105_SB1.G.187 | SB1.G.187 | down | −1.4038798 | 3.54E−06 | P51617 |
| 6 | 6 | 0 | IRAK1 | P51617 | wp107_SB1.G.187 | SB1.G.187 | down | −1.99584368 | 2.94E−07 | P51617 |
| 6 | 6 | 0 | IRAK1 | P51617 | wp172_SB1.G.187 | SB1.G.187 | down | −1.57174723 | 1.01E−05 | P51617 |
| 6 | 6 | 0 | NUAK1 | O60285 | wp017_WH.9533.153 | WH.9533.153 | down | −1.36677906 | 9.35E−05 | O60285 |
| 6 | 6 | 0 | NUAK1 | O60285 | wp076_MFH.5.115.2 | MFH.5.115.2 | down | −0.52635196 | 0.00267708 | O60285 |
| 6 | 6 | 0 | NUAK1 | O60285 | wp076_MFH.5.116.1 | MFH.5.116.1 | down | −0.70779246 | 0.00079062 | O60285 |
| 6 | 6 | 0 | NUAK1 | O60285 | wp076_MFH.5.126.1 | MFH.5.126.1 | down | −1.64913529 | 2.06E−05 | O60285 |
| 6 | 6 | 0 | NUAK1 | O60285 | wp146_TMX.02.138 | TMX.02.138 | down | −0.82541373 | 0.00723896 | O60285 |
| 6 | 6 | 0 | NUAK1 | O60285 | wp146_TMX.02.174 | TMX.02.174 | down | −1.36070521 | 0.00129304 | O60285 |
| 6 | 6 | 0 | MAP3K7 | O43318 | wp056_SB1.G.187 | SB1.G.187 | down | −1.00289771 | 6.74E−06 | O43318 |
| 6 | 6 | 0 | MAP3K7 | O43318 | wp103_SB1.G.187 | SB1.G.187 | down | −1.37390723 | 9.99E−07 | O43318 |
| 6 | 6 | 0 | MAP3K7 | O43318 | wp105_SB1.G.187 | SB1.G.187 | down | −1.03143301 | 1.25E−07 | O43318 |
| 6 | 6 | 0 | MAP3K7 | O43318 | wp107_SB1.G.187 | SB1.G.187 | down | −1.33023832 | 7.08E−08 | O43318 |
| 6 | 6 | 0 | MAP3K7 | O43318 | wp152_DB1114 | DB1114 | down | −0.90263997 | 1.07E−07 | O43318 |
| 6 | 6 | 0 | MAP3K7 | O43318 | wp172_SB1.G.187 | SB1.G.187 | down | −0.57724507 | 0.00619854 | O43318 |
| 5 | 5 | 0 | SNRK | Q9NRH2 | wp080_RSS0680 | RSS0680 | down | −0.55666235 | 0.00123631 | Q9NRH2 |
| 5 | 5 | 0 | SNRK | Q9NRH2 | wp117_RSS0680 | RSS0680 | down | −0.63213226 | 1.40E−05 | Q9NRH2 |
| 5 | 5 | 0 | SNRK | Q9NRH2 | wp134_BS.J.04.178 | BSJ.04.178 | down | −1.52301866 | 4.60E−06 | Q9NRH2 |
| 5 | 5 | 0 | SNRK | Q9NRH2 | wp195_BSJ.05.026 | BSJ.05.026 | down | −1.85636398 | 5.37E−08 | Q9NRH2 |
| 5 | 5 | 0 | SNRK | Q9NRH2 | wp195_JWG.123 | JWG.123 | down | −1.19749601 | 6.19E−07 | Q9NRH2 |
| 5 | 5 | 0 | LRRK2 | Q5S007 | wp160_DB0646_1 hr | DB0646 | down | −1.1650443 | 0.00015255 | Q5S007 |
| 5 | 5 | 0 | LRRK2 | Q5S007 | wp160_DB0646_2 hr | DB0646 | down | −1.18065661 | 0.00014467 | Q5S007 |
| 5 | 5 | 0 | LRRK2 | Q5S007 | wp160_DB0646_4 hr | DB0646 | down | −1.30699127 | 9.64E−05 | Q5S007 |
| 5 | 5 | 0 | LRRK2 | Q5S007 | wp160_DB0646_8 hr | DB0646 | down | −1.74707305 | 3.00E−05 | Q5S007 |
| 5 | 5 | 0 | LRRK2 | Q5S007 | wp160_SK.3.91_1 hr | SK.3.91 | down | −0.84471372 | 0.00054481 | Q5S007 |
| 5 | 5 | 0 | STK4 | Q13043-1 | wp017_WH.9533.153 | WH.9533.153 | down | −0.49356767 | 0.00030667 | Q13043 |
| 5 | 5 | 0 | STK4 | Q13043-1 | wp056_WH.10417.099 | WH.10417.099 | down | −0.71570237 | 7.55E−05 | Q13043 |
| 5 | 5 | 0 | STK4 | Q13043-1 | wp103_WH.10417.099 | WH.10417.099 | down | −0.73874057 | 2.04E−06 | Q13043 |
| 5 | 5 | 0 | STK4 | Q13043-1 | wp107_WH.10417.099 | WH.10417.099 | down | −0.71876513 | 2.13E−05 | Q13043 |
| 5 | 5 | 0 | STK4 | Q13043-1 | wp172_WH.10417.099 | WH.10417.099 | down | −0.59830459 | 0.00315137 | Q13043 |
| 5 | 5 | 0 | BMPR1A | P36894 | wp105_SK.3.91 | SK.3.91 | down | −0.40770173 | 0.00368766 | P36894 |
| 5 | 5 | 0 | BMPR1A | P36894 | wp107_SK.3.91 | SK.3.91 | down | −1.10310636 | 6.76E−06 | P36894 |
| 5 | 5 | 0 | BMPR1A | P36894 | wp131_TL12.186 | TL12.186 | down | −0.83336961 | 8.10E−05 | P36894 |
| 5 | 5 | 0 | BMPR1A | P36894 | wp133_SK.3.91_4 hr | SK.3.91 | down | −0.80066432 | 0.00415562 | P36894 |
| 5 | 5 | 0 | BMPR1A | P36894 | wp137_TL12.186 | TL12.186 | down | −0.65657663 | 0.00424613 | P36894 |
| 5 | 5 | 0 | AKT1 | P31749 | wp104_INY.02.164 | INY.02.164 | down | −0.79259464 | 0.00010785 | P31749 |
| 5 | 5 | 0 | AKT1 | P31749 | wp118_INY.03.041.01 | INY.03.041.01 | down | −2.25794728 | 4.85E−07 | P31749 |
| 5 | 5 | 0 | AKT1 | P31749 | wp135_INY.03.041_01_8 hr | INY.03.041_01 | down | −2.27269829 | 1.39E−10 | P31749 |
| 5 | 5 | 0 | AKT1 | P31749 | wp135_INY.03.041.01_4 hr | INY.03.041.01 | down | −1.22479594 | 9.02E−09 | P31749 |
| 5 | 5 | 0 | AKT1 | P31749 | wp137_INY.02.164 | INY.02.164 | down | −3.20554588 | 2.64E−10 | P31749 |
| 5 | 5 | 0 | RPS6KB1 | P23443-1 | wp080_RSS0680 | RSS0680 | down | −0.43575787 | 0.00099775 | P23443 |
| 5 | 5 | 0 | RPS6KB1 | P23443-1 | wp107_SK.3.91 | SK.3.91 | down | −0.41970503 | 0.00231545 | P23443 |
| 5 | 5 | 0 | RPS6KB1 | P23443-1 | wp117_RSS0680 | RSS0680 | down | −0.38927093 | 0.00020674 | P23443 |
| 5 | 5 | 0 | RPS6KB1 | P23443-1 | wp195_BSJ.05.026 | BSJ.05.026 | down | −0.56288448 | 3.24E−05 | P23443 |
| 5 | 5 | 0 | RPS6KB1 | P23443-1 | wp195_JWG.123 | JWG.123 | down | −0.34257461 | 0.00045083 | P23443 |
| 5 | 5 | 0 | LYN | P07948-2 | wp105_DB0646 | DB0646 | down | −0.9849778 | 3.91E−06 | P07948 |
| 5 | 5 | 0 | LYN | P07948-2 | wp105_SB1.G.187 | SB1.G.187 | down | −1.08714609 | 3.70E−07 | P07948 |
| 5 | 5 | 0 | LYN | P07948-2 | wp114_SB1.G.187 | SB1.G.187 | down | −0.44075204 | 0.00034368 | P07948 |
| 5 | 5 | 0 | LYN | P07948-1 | wp172_DB0646 | DB0646 | down | −0.79380963 | 0.00042027 | P07948 |
| 5 | 5 | 0 | LYN | P07948-1 | wp172_SB1.G.187 | SB1.G.187 | down | −1.229749 | 1.90E−05 | P07948 |
| 5 | 5 | 0 | EPHB6 | O15197-1 | wp071_SB1.G.192.1 | SB1.G.192.1 | down | −0.62590474 | 0.00123846 | O15197 |
| 5 | 5 | 0 | EPHB6 | O15197-1 | wp131_SB1.G.192.1 | SB1.G.192.1 | down | −0.51890056 | 0.00011049 | O15197 |
| 5 | 5 | 0 | EPHB6 | O15197-1 | wp160_DB0646_4 hr | DB0646 | down | −0.3395856 | 0.00079389 | O15197 |
| 5 | 5 | 0 | EPHB6 | O15197-1 | wp160_DB0646_8 hr | DB0646 | down | −0.65476675 | 5.87E−05 | O15197 |
| 5 | 5 | 0 | EPHB6 | O15197-1 | wp168_DB0662 | DB0662 | down | −0.73595936 | 0.00427583 | O15197 |
| 4 | 4 | 0 | BAZ1B | Q9UIG0 | wp080_RSS0680 | RSS0680 | down | −0.40612878 | 5.28E−05 | Q9UIG0 |
| 4 | 4 | 0 | BAZ1B | Q9UIG0 | wp084_BSJ.03.123 | BSJ.03.123 | down | −0.35322157 | 0.00015651 | Q9UIG0 |
| 4 | 4 | 0 | BAZ1B | Q9UIG0 | wp084_BSJ.04.132 | BSJ.04.132 | down | −0.32218275 | 0.00209898 | Q9UIG0 |
| 4 | 4 | 0 | BAZ1B | Q9UIG0 | wp147_DD.03.107_4 uM | DD.03.107 | down | −0.3417724 | 6.64E−05 | Q9UIG0 |
| 4 | 4 | 0 | IRAK4 | Q9NWZ3-1 | wp108_FMF.06.098.1 | FMF.06.098.1 | down | −0.6504927 | 3.34E−05 | Q9NWZ3 |
| 4 | 4 | 0 | IRAK4 | Q9NWZ3-1 | wp131_FMF.06.098.1 | FMF.06.098.1 | down | −0.70858699 | 1.42E−05 | Q9NWZ3 |
| 4 | 4 | 0 | IRAK4 | Q9NWZ3-1 | wp131_TL12.186 | TL12.186 | down | −0.42925526 | 1.04E−05 | Q9NWZ3 |
| 4 | 4 | 0 | IRAK4 | Q9NWZ3-1 | wp137_TL12.186 | TL12.186 | down | −0.41851821 | 5.57E−06 | Q9NWZ3 |
| 4 | 4 | 0 | PIP5K1A | Q99755-3 | wp107_SK.3.91 | SK.3.91 | down | −0.45004247 | 1.82E−05 | Q99755 |
| 4 | 4 | 0 | PIP5K1A | Q99755-3 | wp131_TL12.186 | TL12.186 | down | −0.343588 | 0.00093525 | Q99755 |
| 4 | 4 | 0 | PIP5K1A | Q99755-3 | wp133_SK.3.91_4 hr | SK.3.91 | down | −0.37122886 | 0.00021934 | Q99755 |
| 4 | 4 | 0 | PIP5K1A | Q99755-3 | wp137_TL12.186 | TL12.186 | down | −0.45256793 | 0.0006106 | Q99755 |
| 4 | 2 | 2 | PFKFB4 | Q16877 | wp071_FMF.04.135.1 | FMF.04.135.1 | down | −0.49690856 | 0.00527403 | Q16877 |
| 4 | 2 | 2 | PFKFB4 | Q16877 | wp080_RSS0680 | RSS0680 | down | −0.33887596 | 0.00180023 | Q16877 |
| 4 | 4 | 0 | CDK10 | Q15131-1 | wp076_MFH.6.103.1 | MFH.5.103.1 | down | −0.69882393 | 0.0080645 | Q15131 |
| 4 | 4 | 0 | CDK10 | Q15131-1 | wp131_TL12.186 | TL12.186 | down | −0.93504794 | 2.50E−05 | Q15131 |
| 4 | 4 | 0 | CDK10 | Q15131-1 | wp133_SK.3.91_4 hr | SK.3.91 | down | −0.54401759 | 0.00156763 | Q15131 |
| 4 | 4 | 0 | CDK10 | Q15131-1 | wp134_BSJ.04.178 | BSJ.04.178 | down | −0.65464953 | 0.00098313 | Q15131 |
| 4 | 4 | 0 | PRKAA2 | P54646 | wp105_SK.3.91 | SK.3.91 | down | −0.90288121 | 2.90E−06 | P54646 |
| 4 | 4 | 0 | PRKAA2 | P54646 | wp146_TMX.02.138 | TMX.02.138 | down | −1.47567521 | 0.00029302 | P54646 |
| 4 | 4 | 0 | PRKAA2 | P54646 | wp146_TMX.02.174 | TMX.02.174 | down | −1.82606724 | 0.00013554 | P54646 |
| 4 | 4 | 0 | PRKAA2 | P54646 | wp172_SK.3.91 | SK.3.91 | down | −0.92603174 | 1.15E−06 | P54646 |
| 4 | 4 | 0 | CDK14 | O94921-1 | wp017_WH.9533.153 | WH.9533.153 | down | −0.46368499 | 0.00116641 | O94921 |
| 4 | 4 | 0 | CDK14 | O94921-1 | wp076_MFH.5.126.1 | MFH.5.126.1 | down | −0.34134638 | 0.00489961 | O94921 |
| 4 | 4 | 0 | CDK14 | O94921-1 | wp172_SB1.G.187 | SB1.G.187 | down | −1.52491273 | 0.00023303 | O94921 |
| 4 | 4 | 0 | CDK14 | O94921-1 | wp172_SK_3.91 | SK.3.91 | down | −0.81755386 | 0.00070835 | O94921 |
| 4 | 4 | 0 | RIPK2 | O43353-1 | wp105_DB0646 | DB0646 | down | −0.42154218 | 0.00994771 | O43353 |
| 4 | 4 | 0 | RIPK2 | O43353-1 | wp105_SB1.G.187 | SB1.G.187 | down | −0.57201652 | 0.00294293 | O43353 |
| 4 | 4 | 0 | RIPK2 | O43353-1 | wp114_SB1.G.187 | SB1.G.187 | down | −0.88279998 | 0.00073787 | O43353 |
| 4 | 4 | 0 | RIPK2 | O43353-1 | wp172_DB0646 | DB0646 | down | −0.47793987 | 0.00651176 | O43353 |
| 3 | 3 | 0 | SIK3 | Q9Y2K2 | wp168_DB1113 | DB1113 | down | −1.60215807 | 2.66E−08 | Q9Y2K2 |
| 3 | 3 | 0 | SIK3 | Q9Y2K2 | wp195_BSJ.05.026 | BSJ.05.026 | down | −1.18627938 | 6.15E−07 | Q9Y2K2 |
| 3 | 3 | 0 | SIK3 | Q9Y2K2 | wp195_JWG.123 | JWG.123 | down | −1.32219884 | 3.36E−07 | Q9Y2K2 |
| 3 | 3 | 0 | PIM2 | Q9P1W9 | wp017_WH.9533.153 | WH.9533.153 | down | −0.49358813 | 0.00058166 | Q9P1W9 |
| 3 | 3 | 0 | PIM2 | Q9P1W9 | wp076_MFH.5.115.2 | MFH.5.115.2 | down | −1.67913358 | 0.00023725 | Q9P1W9 |
| 3 | 3 | 0 | PIM2 | Q9P1W9 | wp076_MFH.5.126.1 | MFH.5.126.1 | down | −3.01918335 | 1.85E−05 | Q9P1W9 |
| 3 | 3 | 0 | CAMKK1 | Q8N5S9-2 | wp045_DB0614 | DB0614 | down | −0.64866074 | 0.00030494 | Q8N5S9 |
| 3 | 3 | 0 | CAMKK1 | Q8N589-2 | wp045_RSS0628 | RSS0628 | down | −0.34119233 | 0.00429119 | Q8N5S9 |
| 3 | 3 | 0 | CAMKK1 | Q8N5S9 | wp117_DB0614 | DB0614 | down | −0.47098697 | 0.0005669 | Q8N5S9 |
| 3 | 3 | 0 | PDIK1L | Q8N165 | wp152_DB1114 | DB1114 | down | −0.48201074 | 0.00138694 | Q8N165 |
| 3 | 3 | 0 | PDIK1L | Q8N165 | wp168_DB0662 | DB0662 | down | −0.32715938 | 0.00019184 | Q8N165 |
| 3 | 3 | 0 | PDIK1L | Q8N165 | wp168_DB1113 | DB1113 | down | −0.53343833 | 1.70E−05 | Q8N165 |
| 3 | 3 | 0 | SBK1 | Q52WX2 | wp107_SK.3.91 | SK.3.91 | down | −0.92103779 | 0.00144983 | Q52WX2 |
| 3 | 3 | 0 | SBK1 | Q52WX2 | wp133_SK.3.91_2 hr | SK.3.91 | down | −0.68524472 | 0.0003185 | Q52WX2 |
| 3 | 3 | 0 | SBK1 | Q52WVX2 | wp133_SK.3.91_4 hr | SK.3.91 | down | −0.90292165 | 7.62E−05 | Q52WX2 |
| 3 | 3 | 0 | STK38 | Q15208 | wp137_TL13.97 | TL13.97 | down | −0.39116144 | 5.99E−05 | Q15208 |
| 3 | 3 | 0 | STK38 | Q15208 | wp146_TMX.02.138 | TMX.02.138 | down | −0.34759547 | 0.00205421 | Q15208 |
| 3 | 3 | 0 | STK38 | Q15208 | wp146_TMX.02.174 | TMX.02.174 | down | −0.41171038 | 0.00113192 | Q15208 |
| 3 | 3 | 0 | MAP2K5 | Q13163-1 | wp071_SB1.G.181 | SB1.G.181 | down | −1.13782841 | 0.00013055 | Q13163 |
| 3 | 3 | 0 | MAP2K5 | Q13163-1 | wp071_SB1.G.200 | SB1.G.200 | down | −1.43326011 | 4.33E−05 | Q13163 |
| 3 | 3 | 0 | MAP2K5 | Q13163-1 | wp074_dRAF.3 | dRAF.3 | down | −0.5935186 | 0.00644494 | Q13163 |
| 3 | 2 | 1 | BTK | Q06187-2 | wp055_DD.03.171 | DD.03.171 | down | −3.07718518 | 3.29E−10 | Q06187 |
| 3 | 2 | 1 | BTK | Q06187-2 | wp055_DD.04.118 | DD.04.118 | down | −3.26333718 | 1.28E−09 | Q06187 |
| 3 | 3 | 0 | AK2 | P54819 | wp056_SB1.G.187 | SB1.G.187 | down | −0.36908967 | 0.00077404 | P54819 |
| 3 | 3 | 0 | AK2 | P54819 | wp084_BS.J.03.123 | BSJ.03.123 | down | −0.39499146 | 0.00039356 | P54819 |
| 3 | 3 | 0 | AK2 | P54819 | wp084_BSJ.04.132 | BSJ.04.132 | down | −0.33225916 | 0.00078471 | P54819 |
| 3 | 3 | 0 | HK2 | P52789 | wp076_MFH.5.103.1 | MFH.5.103.1 | down | −0.38187345 | 0.00301564 | P52789 |
| 3 | 3 | 0 | HK2 | P52789 | wp076_MFH.5.116.1 | MFH.5.116.1 | down | −0.43472855 | 0.0017839 | P52789 |
| 3 | 3 | 0 | HK2 | P52789 | wp076_MFH.5.126.1 | MFH.5.126.1 | down | −0.32201934 | 0.00589619 | P52789 |
| 3 | 3 | 0 | CSNK1A1 | P48729-2 | wp076_MFH.5.115.2 | MFH.5.115.2 | down | −0.3489851 | 0.00131714 | P48729 |
| 3 | 3 | 0 | CSNK1A1 | P48729-2 | wp076_MFH.5.126.1 | MFH.5.126.1 | down | −0.4586371 | 0.0004181 | P48729 |
| 3 | 3 | 0 | CSNK1A1 | P48729-2 | wp107_WH.10417.099 | WH.10417.099 | down | −2.4280139 | 1.20E−09 | P48729 |
| 3 | 3 | 0 | EPHB2 | P29323 | wp107_SK.3.91 | SK.3.91 | down | −0.62930104 | 0.00115023 | P29323 |
| 3 | 3 | 0 | EPHB2 | P29323 | wp195_BJG.04.203 | BJG.04.203 | down | −0.45354103 | 0.00147688 | P29323 |
| 3 | 3 | 0 | EPHB2 | P29323 | wp195_JWG.148 | JWG.148 | down | −0.3595887 | 0.00444441 | P29323 |
| 3 | 3 | 0 | EIF2AK2 | P19525 | wp117_DB0614 | DB0614 | down | −0.32208952 | 0.00012192 | P19525 |
| 3 | 3 | 0 | EIF2AK2 | P19525 | wp147_DD.03.106_2 uM | DD.03.106 | down | −0.35450156 | 1.06E−05 | P19525 |
| 3 | 3 | 0 | EIF2AK2 | P19525 | wp147_DD.03.108_4 uM | DD.03.106 | down | −0.40423566 | 5.43E−06 | P19525 |
| 3 | 1 | 2 | TK1 | P04183 | wp168_DB0661 | DB0661 | down | −0.35232633 | 0.00024376 | P04183 |
| 3 | 3 | 0 | RPS6KA4 | O75676 | wp108_FMF.06.098.1 | FMF.06.098.1 | down | −0.41963381 | 0.00022764 | O75676 |
| 3 | 3 | 0 | RPS6KA4 | O75676 | wp131_FMF.06.098.1 | FMF.06.098.1 | down | −0.53513038 | 2.78E−05 | O75676 |
| 3 | 3 | 0 | RPS6KA4 | O75676 | wp146_TMX.02.174 | TMX.02.174 | down | −1.50028904 | 0.00411744 | O75676 |
| 3 | 3 | 0 | CHEK1 | 014757 | wp076_MFH.5.126.1 | MFH.5.126.1 | down | −0.36063559 | 0.00353339 | O14757 |
| 3 | 3 | 0 | CHEK1 | 014757 | wp108_FMF.06.098.1 | FMF.06.098.1 | down | −0.36743997 | 0.00031477 | O14757 |
| 3 | 3 | 0 | CHEK1 | O14757 | wp114_FMF.06.098.1 | FMF.06.098.1 | down | −0.41242147 | 0.00121422 | O14757 |
| 3 | 2 | 1 | NME4 | O00746 | wp084_BSJ.03.123 | BSJ.03.123 | down | −0.36809137 | 0.00318879 | O00746 |
| 3 | 2 | 1 | NME4 | O00746 | wp084_BSJ.04.132 | BSJ.04.132 | down | −0.44667367 | 0.00053277 | O00746 |
| 3 | 3 | 0 | CDC7 | O00311-1 | wp108_FMF.06.098.1 | FMF.06.098.1 | down | −0.3322973 | 0.00382399 | O00311 |
| 3 | 3 | 0 | CDC7 | O00311-1 | wp131_FMF.06.098.1 | FMF.06.098.1 | down | −0.4109493 | 0.00120241 | O00311 |
| 3 | 3 | 0 | CDC7 | O00311-1 | wp133_SK.3.91_4 hr | SK.3.91 | down | −0.3651703 | 0.00064216 | O00311 |
| 2 | 2 | 0 | IRAK3 | Q9Y616 | wp146_TMX.02.138 | TMX.02.138 | down | −1.54363112 | 0.00046405 | Q9Y616 |
| 2 | 2 | 0 | IRAK3 | Q9Y616 | wp146_TMX.02.174 | TMX.02.174 | down | −1.34339955 | 0.00076303 | Q9Y616 |
| 2 | 2 | 0 | CDK11A | Q9UQ88-1 | wp068_FMF.04.147.1 | FMF.04.147.1 | down | −0.73860839 | 0.00351191 | Q9UQ88 |
| 2 | 2 | 0 | CDK11A | Q9UQ88-1 | wp105_DB0646 | DB0646 | down | −0.86800377 | 0.00130257 | Q9UQ88 |
| 2 | 2 | 0 | TAOK2 | Q9UL54-2 | wp160_DB0646_8 hr | DB0646 | down | −0.59727614 | 6.49E−05 | Q9UL54 |
| 2 | 2 | 0 | TAOK2 | Q9UL54 | wp152_DB1114 | DB1114 | down | −0.32399577 | 0.00084471 | Q9UL54 |
| 2 | 1 | 1 | PANK3 | Q9H999 | wp152_DB0646 | DB0646 | down | −0.35335162 | 0.00438757 | Q9H999 |
| 2 | 2 | 0 | TPK1 | Q9H3S4 | wp114_DB0646 | DB0646 | down | −1.14373314 | 0.00209237 | Q9H3S4 |
| 2 | 2 | 0 | TPK | Q9H3S4 | wp114_FMF.06.098.1 | FMF.06.098.1 | down | −0.87441291 | 0.00570475 | Q9H3S4 |
| 2 | 2 | 0 | TAOK3 | Q9H2K8 | wp103_SB1.G.187 | SB1.G.187 | down | −0.37265131 | 0.00075821 | Q9H2K8 |
| 2 | 2 | 0 | TAOK3 | Q9H2K8 | wp107_SB1.G.187 | SB1.G.187 | down | −0.38577524 | 0.00033926 | Q9H2K8 |
| 2 | 2 | 0 | RPS6KC1 | Q96S38 | wp105_SK.3.91 | SK.3.91 | down | −0.33267469 | 0.00020905 | Q96S38 |
| 2 | 2 | 0 | RPS6KC1 | Q96S38 | wp107_SK.3.91 | SK.3.91 | down | −0.36229324 | 0.0021567 | Q96S38 |
| 2 | 2 | 0 | COQ8B | Q96D53-1 | wp137_DD.03.106 | DD.03.106 | down | −0.34423313 | 0.00527359 | Q96D53 |
| 2 | 2 | 0 | COQ8B | Q96D53-1 | wp137_TL12.186 | TL12.186 | down | −0.49336612 | 0.00094704 | Q96D53 |
| 2 | 1 | 1 | SGK3 | Q96BR1-1 | wp068_FMF.04.147.1 | FMF.04.147.1 | down | −0.3389233 | 0.00141812 | Q96BR1 |
| 2 | 2 | 0 | IPMK | Q8NFU5 | wp056_WH.10417.099 | WH.10417.099 | down | −0.58453266 | 0.00349742 | Q8NFU5 |
| 2 | 2 | 0 | IPMK | Q8NFU5 | wp107_WH.10417.099 | WH.10417.099 | down | −0.4153621 | 0.00284699 | Q8NFU5 |
| 2 | 2 | 0 | MAP3K21 | Q5TCX8 | wp131_TL12.186 | TL12.186 | down | −0.96904108 | 9.27E−05 | Q5TCX8 |
| 2 | 2 | 0 | MAP3K21 | Q5TCX8 | wp133_SK.3.91_4 hr | SK.3.91 | down | −0.46848396 | 0.00767573 | Q5TCX8 |
| 2 | 2 | 0 | MAPKAPK3 | Q16644 | wp152_DB1114 | DB1114 | down | −1.95166313 | 2.64E−08 | Q16644 |
| 2 | 2 | 0 | MAPKAPK3 | Q16644 | wp168_DB1113 | DB1113 | down | −3.12707558 | 3.11E−09 | Q16644 |
| 2 | 2 | 0 | MAPK11 | Q15759 | wp105_DB0646 | DB0646 | down | −0.39979862 | 0.00775999 | Q15759 |
| 2 | 2 | 0 | MAPK11 | Q15759 | wp114_DB0646 | DB0646 | down | −0.62087683 | 0.0031641 | Q15759 |
| 2 | 2 | 0 | PTK6 | Q13882 | wp114_FMF.06.098.1 | FMF.06.098.1 | down | −0.44014855 | 0.00119636 | Q13882 |
| 2 | 2 | 0 | PTK6 | Q13882 | wp114_SK_3.91 | SK.3.91 | down | −0.41708012 | 0.0014731 | Q13882 |
| 2 | 2 | 0 | CDK18 | Q07002-3 | wp105_SK.3.91 | SK.3.91 | down | −1.23289594 | 0.00016013 | Q07002 |
| 2 | 2 | 0 | CDK18 | Q07002-3 | wp114_SK.3.91 | SK.3.91 | down | −0.82945035 | 0.00059556 | Q07002 |
| 2 | 2 | 0 | MAPK10 | P53779 | wp104_TMX.01.160.1_0.25 | TMX.01.160.1 | down | −0.57430662 | 0.00070736 | P53779 |
| 2 | 2 | 0 | MAPK10 | P53779 | wp104_TMX.01.160.1_1 | TMX.01.160.1 | down | −0.57433733 | 0.00070718 | P53779 |
| 2 | 2 | 0 | MAPK12 | P53778 | wp105_DB0646 | DB0646 | down | −0.59387369 | 5.41E−05 | P53778 |
| 2 | 2 | 0 | MAPK12 | P53778 | wp172_DB0646 | DB0646 | down | −1.4571321 | 0.00087363 | P53778 |
| 2 | 2 | 0 | PIK3CG | P48736 | wp068_FMF.04.147.1 | FMF.04.147.1 | down | −0.35813172 | 0.0001378 | P48736 |
| 2 | 2 | 0 | PIK3CG | P48736 | wp076_MFH.6.126.1 | MFH.5.126.1 | down | −0.37353765 | 0.00095918 | P48736 |
| 2 | 2 | 0 | EPHA2 | P29317 | wp114_SB1.G.187 | SB1.G.187 | down | −0.40104248 | 0.00255044 | P29317 |
| 2 | 2 | 0 | EPHA2 | P29317 | wp172_SB1.G.187 | SB1.G.187 | down | −0.41071183 | 0.0090955 | P29317 |
| 2 | 2 | 0 | MARK3 | P27448-5 | wp134_BSJ.04.178 | BSJ.04.178 | down | −0.49772035 | 1.84E−05 | P27448 |
| 2 | 2 | 0 | MARK3 | P27448-5 | wp195_BSJ.05.026 | BSJ.05.026 | down | −0.82423351 | 7.10E−07 | P27448 |
| 2 | 2 | 0 | EPHA1 | P21709 | wp114_FMF.08.098.1 | FMF.06.098.1 | down | −0.42202651 | 0.00055575 | P21709 |
| 2 | 2 | 0 | EPHA1 | P21709 | wp114_SB1.G.187 | SB1.G.187 | down | −0.35515795 | 0.00109162 | P21709 |
| 2 | 2 | 0 | HK1 | P19367-3 | wp080_RSS0680 | RSS0680 | down | −0.36955722 | 0.00317073 | P19367 |
| 2 | 2 | 0 | HK1 | P19367-3 | wp084_BS.J.03.123 | BSJ.03.123 | down | −0.34842928 | 6.70E−05 | P19367 |
| 2 | 1 | 1 | ERN1 | O75460 | wp076_MFH.5.126.1 | MFH.5.126.1 | down | −0.48816302 | 0.00222245 | O75460 |
| 2 | 2 | 0 | MAST3 | O60307 | wp081_TL13.178 | TL13.178 | down | −0.48522701 | 0.00358858 | O60307 |
| 2 | 2 | 0 | MAST3 | O60307 | wp107_SK.3.91 | SK.3.91 | down | −0.49836904 | 0.00144919 | O60307 |
| 2 | 1 | 1 | PDXK | O00764-1 | wp117_DB0614 | DB0614 | down | −0.39644982 | 0.0001717 | O00764 |
| 1 | 1 | 0 | RPSGKA6 | Q9UK32 | wp105_SK.3.91 | SK.3.91 | down | −0.83004221 | 0.00083597 | Q9UK32 |
| 1 | 1 | 0 | TBK1 | Q9UHD2 | wp146_TMX.02.174 | TMX.02.174 | down | −0.59509307 | 0.00079098 | Q9UHD2 |
| 1 | 1 | 0 | EIF2AK4 | Q9P2K8-1 | wp080_RSS0680 | RSS0680 | down | −0.33113885 | 0.00011881 | Q9P2K8 |
| 1 | 1 | 0 | ETNK1 | Q9HBU6-1 | wp088_SB1.G.194 | SB1.G.194 | down | −0.3662199 | 0.00868136 | Q9HBU6 |
| 1 | 1 | 0 | UCK2 | Q9BZX2-1 | wp160_DB0646_8 hr | DB0646 | down | −0.32459671 | 0.00033804 | Q9BZX2 |
| 1 | 1 | 0 | PKMYT1 | Q99640 | wp131_SB1.G.192.1 | SB1.G.192.1 | down | −0.73009233 | 0.00077015 | Q99640 |
| 1 | 1 | 0 | TESK2 | Q96S53-1 | wp147_DD.03.106_4 uM | DD.03.106 | down | −0.38306443 | 0.00805456 | Q96S53 |
| 1 | 1 | 0 | TRIB3 | Q96RU7 | wp114_SK.3.91 | SK.3.91 | down | −1.21106356 | 0.0075444 | Q96RU7 |
| 1 | 1 | 0 | TRPM7 | Q96QT4 | wp107_SK.3.91 | SK.3.91 | down | −0.32410264 | 0.00252215 | Q96QT4 |
| 1 | 1 | 0 | NEK1 | Q96PY6-3 | wp146_TMX.02.174 | TMX.02.174 | down | −0.38708398 | 0.00647863 | Q96PY6 |
| 1 | 1 | 0 | ITPKC | Q96DU7 | wp133_SK.3.91_4 hr | SK.3.91 | down | −0.35562928 | 0.00084004 | Q96DU7 |
| 1 | 1 | 0 | HASPIN | QSTF76-1 | wp137_TL12.186 | TL12.186 | down | −0.36804824 | 0.00054567 | QSTF76 |
| 1 | 1 | 0 | TBCK | QSTEA7 | wp056_ZNL.02.096 | ZNL.02.096 | down | −0.33343559 | 0.00504445 | QSTEA7 |
| 1 | 1 | 0 | CERK | Q8TCT0 | wp131_TL12.186 | TL12.186 | down | −0.33434871 | 0.00233089 | Q8TCT0 |
| 1 | 1 | 0 | MINK1 | Q8N408-1 | wp107_WH.10417.099 | WH.10417.099 | down | −0.56605322 | 0.00278694 | Q8N4C8 |
| 1 | 1 | 0 | MAPKAPK5 | Q8IW41 | wp080_RSS0680 | RSS0680 | down | −0.33553471 | 0.00309437 | Q81W41 |
| 1 | 1 | 0 | HIPK1 | Q86Z02 | wp133_SK.3.91_4 hr | SK.3.91 | down | −0.40092948 | 0.00093996 | Q86Z02 |
| 1 | 1 | 0 | STK32C | Q86UX6 | wp114_SK.3.91 | SK.3.91 | down | −0.61889541 | 0.00473885 | Q86UX6 |
| 1 | 1 | 0 | PKN3 | Q6P5Z2 | wp114_FMF.06.098.1 | FMF.06.098.1 | down | −0.45102208 | 0.00783628 | Q6P5Z2 |
| 1 | 1 | 0 | DDR2 | Q16832 | wp105_DB0646 | DB0646 | down | −0.47407396 | 0.00029656 | Q16832 |
| 1 | 1 | 0 | DGKD | Q16760-1 | wp045_DB0663 | DB0663 | down | −0.59553573 | 0.00620305 | Q16760 |
| 1 | 1 | 0 | MELK | Q14680 | wp131_FMF.06.098.1 | FMF.06.098.1 | down | −0.37999062 | 6.94E−05 | Q14680 |
| 1 | 1 | 0 | TNK1 | Q13470-2 | wp114_SK.3.91 | SK.3.91 | down | −0.73935291 | 0.00014822 | Q13470 |
| 1 | 1 | 0 | MAP3K12 | Q12852-2 | wp105_SK.3.91 | SK.3.91 | down | −0.69312068 | 0.0001154 | Q12852 |
| 1 | 1 | 0 | GALK2 | Q01415-1 | wp056_ZNL.02.096 | ZNL.02.096 | down | −0.37239714 | 0.00822976 | Q01415 |
| 1 | 1 | 0 | EPHB4 | P54760 | wp172_SK.3.91 | SK.3.91 | down | −0.50585237 | 0.00325831 | P54760 |
| 1 | 1 | 0 | EPHB3 | P54753 | wp114_DB0646 | DB0646 | down | −0.41745479 | 0.00171991 | P54753 |
| 1 | 1 | 0 | DAPK1 | P53355-3 | wp076_MFH.5.126.1 | MFH.5.126.1 | down | −0.44876435 | 0.00898669 | P53355 |
| 1 | 1 | 0 | DGKE | P52429 | wp056_ZNL.02.096 | ZNL.02.096 | down | −0.35121446 | 0.00546424 | P52429 |
| 1 | 1 | 0 | NEK4 | P51957 | wp146_TMX.02.174 | TMX.02.174 | down | −0.95084627 | 0.00053548 | P51957 |
| 1 | 1 | 0 | NEK3 | P51956 | wp146_TMX.02.174 | TMX.02.174 | down | −0.84969663 | 0.00126406 | P51956 |
| 1 | 1 | 0 | PRKCI | P41743 | wp056_ZNL.02.096 | ZNL.02.096 | down | −0.53649759 | 0.00070684 | P41743 |
| 1 | 1 | 0 | TGFBR1 | P36897-2 | wp107_SK.3.91 | SK.3.91 | down | −0.52638815 | 0.001917 | P36897 |
| 1 | 1 | 0 | TYK2 | P29597 | wp195_JWG.148 | JWG.148 | down | −0.51865142 | 0.00013389 | P29597 |
| 1 | 1 | 0 | FGFR2 | P21802-11 | wp088_SB1.G.194 | SB1.G.194 | down | −0.48040699 | 0.00803761 | P21802 |
| 1 | 1 | 0 | FLT1 | P17948-1 | wp131_TL12.186 | TL12.186 | down | −0.69898705 | 0.00111329 | P17948 |
| 1 | 1 | 0 | FGFR1 | P11362-21 | wp108_FMF.06.098.1 | FMF.06.098.1 | down | −0.41205594 | 0.0053701 | P11362 |
| 1 | 1 | 0 | CIT | O14578 | wp152_DB1114 | DB1114 | down | −0.38996128 | 1.22E−06 | O14578 |
| 1 | 1 | 0 | EEF2K | O00418 | wp137_TL12.186 | TL12.186 | down | −0.34432662 | 0.00026004 | O00418 |
| 1 | 1 | 0 | HYKK | A2RU49 | wp117_RSS0660 | RSS0680 | down | −0.34757913 | 0.00313693 | A2RU49 |

APPENDIX I

| Freq | Freq.down | Freq.up | ID | Accession | Experiment.ID | Compound | direction.of.change | logFC | P.Value |
|---|---|---|---|---|---|---|---|---|---|
| 59 | 58 | 1 | CDK4 | P11802 | wp005_YKL.6.102 | YKL.6.102 | down | −0.641951217 | 5.95E−06 |
| 59 | 58 | 1 | CDK4 | P11802 | wp005_BSJ.01.147 | BSJ.01.147 | down | −1.41499961 | 6.50E−06 |
| 59 | 58 | 1 | CDK4 | P11802 | wp016_SK.3.91_1 uM | SK.3.91 | down | −1.428531073 | 0.00012439 |
| 59 | 58 | 1 | CDK4 | P11802 | wp016_TL12.186 | TL12.186 | down | −0.916610432 | 0.000807681 |
| 59 | 58 | 1 | CDK4 | P11802 | wp016_SK.3.93 | SK.3.93 | down | −1.30450325 | 0.000183197 |
| 59 | 58 | 1 | CDK4 | P11802 | wp016_SK.3.91 | SK.3.91 | down | −1.160981002 | 0.000300269 |
| 59 | 58 | 1 | CDK4 | P11802 | wp016_LT2.49 | LT2.49 | down | −0.898500628 | 0.000877225 |
| 59 | 58 | 1 | CDK4 | P11802 | wp016_SK.3.89 | SK.3.89 | down | −0.517709528 | 0.007816584 |
| 59 | 58 | 1 | CDK4 | P11802 | wp045_DB0646 | DB0646 | down | −0.90115183 | 4.07E−05 |
| 59 | 58 | 1 | CDK4 | P11802 | wp045_DB0663 | DB0663 | down | −0.682262855 | 0.000135722 |
| 59 | 58 | 1 | CDK4 | P11802 | wp045_RSS0628 | RSS0628 | down | −1.422329067 | 5.56E−06 |
| 59 | 58 | 1 | CDK4 | P11802 | wp045_DB0614 | DB0614 | down | −1.539398185 | 3.93E−06 |
| 59 | 58 | 1 | CDK4 | P11802 | wp047_DD.03.106 | DD.03.106 | down | −0.927722536 | 4.93E−05 |
| 59 | 58 | 1 | CDK4 | P11802 | wp076_MFH.5.126.1 | MFH.5.126.1 | down | −0.607974857 | 0.000956916 |
| 59 | 58 | 1 | CDK4 | P11802 | wp076_MFH.5.115.2 | MFH.5.115.2 | down | −0.724605154 | 0.000456854 |
| 59 | 58 | 1 | CDK4 | P11802 | wp080_RSS0680 | RSS0680 | down | −1.724432192 | 7.44E−07 |
| 59 | 58 | 1 | CDK4 | P11802 | wp081_TL13.178 | TL13.178 | down | −0.57620467 | 1.18E−05 |
| 59 | 58 | 1 | CDK4 | P11802 | wp084_BSJ.01.147 | BSJ.01.147 | down | −1.800381164 | 2.20E−08 |
| 59 | 58 | 1 | CDK4 | P11802 | wp084_BSJ.04.132 | BSJ.04.132 | down | −0.904613523 | 8.29E−07 |
| 59 | 58 | 1 | CDK4 | P11802 | wp084_BSJ.03.204 | BSJ.03.204 | down | −2.161939175 | 4.35E−08 |
| 59 | 58 | 1 | CDK4 | P11802 | wp084_BSJ.03.123 | BSJ.03.123 | down | −0.949602297 | 4.75E−07 |
| 59 | 58 | 1 | CDK4 | P11802 | wp103_DB0646 | DB0646 | down | −0.501482596 | 0.000597794 |
| 59 | 58 | 1 | CDK4 | P11802 | wp103_SK.3.91 | SK.3.91 | down | −1.029179101 | 2.40E−05 |
| 59 | 58 | 1 | CDK4 | P11802 | wp104_TMX.01.160.1_0.25 | TMX.01.160.1 | down | −0.554154379 | 0.000140928 |
| 59 | 58 | 1 | CDK4 | P11802 | wp104_TMX.01.160.1_1 | TMX.01.160.1 | down | −0.767622777 | 2.78E−05 |
| 59 | 58 | 1 | CDK4 | P11802 | wp105_SK.3.91 | SK.3.91 | down | −0.799483848 | 1.52E−06 |
| 59 | 58 | 1 | CDK4 | P11802 | wp107_SK.3.91 | SK.3.91 | down | −1.272417657 | 3.97E−06 |
| 59 | 58 | 1 | CDK4 | P11802 | wp107_DB0646 | DB0646 | down | −0.556524894 | 9.79E−05 |
| 59 | 58 | 1 | CDK4 | P11802 | wp108_FMF.06.098.1 | FMF.06.098.1 | down | −0.873052345 | 1.33E−05 |
| 59 | 58 | 1 | CDK4 | P11802 | wp114_SK.3.91 | SK.3.91 | down | −0.560500364 | 0.000596235 |
| 59 | 58 | 1 | CDK4 | P11802 | wp114_FMF.06.098.1 | FMF.06.098.1 | down | −0.378116595 | 0.002729099 |
| 59 | 58 | 1 | CDK4 | P11802 | wp117_RSS0680 | RSS0680 | down | −1.6785872 | 2.84E−06 |
| 59 | 58 | 1 | CDK4 | P11802 | wp117_DB0614 | DB0614 | down | −1.538525344 | 1.84E−07 |
| 59 | 58 | 1 | CDK4 | P11802 | wp117_DB0646 | DB0646 | down | −0.667307003 | 7.44E−06 |
| 59 | 58 | 1 | CDK4 | P11802 | wp118_INY.03.041.01 | INY.03.041.01 | down | −0.401879289 | 0.000114088 |
| 59 | 58 | 1 | CDK4 | P11802 | wp131_FMF.06.098.1 | FMF.06.098.1 | down | −1.028896899 | 6.67E−06 |
| 59 | 58 | 1 | CDK4 | P11802 | wp131_TL12.186 | TL12.186 | down | −1.186043659 | 3.88E−07 |
| 59 | 58 | 1 | CDK4 | P11802 | wp133_SK.3.91_2 hr | SK.3.91 | down | −0.748354099 | 1.50E−07 |
| 59 | 58 | 1 | CDK4 | P11802 | wp133_SK.3.91_4 hr | SK.3.91 | down | −1.291703777 | 3.08E−09 |
| 59 | 58 | 1 | CDK4 | P11802 | wp135_INY.03.041_01_8 hr | INY.03.041_01 | down | −0.328139921 | 0.000181509 |
| 59 | 58 | 1 | CDK4 | P11802 | wp137_DD.03.106 | DD.03.106 | down | −0.888549761 | 4.37E−07 |
| 59 | 58 | 1 | CDK4 | P11802 | wp137_TL12.186 | TL12.186 | down | −1.1726108 | 9.33E−08 |
| 59 | 58 | 1 | CDK4 | P11802 | wp137_BSJ.03.204 | BSJ.03.204 | down | −2.117716649 | 3.45E−09 |
| 59 | 58 | 1 | CDK4 | P11802 | wp137_TL13.97 | TL13.97 | down | −0.967560314 | 2.72E−07 |
| 59 | 58 | 1 | CDK4 | P11802 | wp147_DD.03.106_1 uM | DD.03.106 | down | −0.69608249 | 1.61E−06 |
| 59 | 58 | 1 | CDK4 | P11802 | wp147_DD.03.106_2 uM | DD.03.106 | down | −0.983540408 | 2.73E−07 |
| 59 | 58 | 1 | CDK4 | P11802 | wp147_DD.03.106_4 uM | DD.03.106 | down | −0.726041996 | 1.30E−06 |
| 59 | 58 | 1 | CDK4 | P11802 | wp152_DB0646 | DB0646 | down | −0.736037536 | 1.02E−05 |
| 59 | 58 | 1 | CDK4 | P11802 | wp152_DB1114 | DB1114 | down | −0.672309357 | 1.66E−05 |
| 59 | 58 | 1 | CDK4 | P11802 | wp152_DB118430 | DB118430 | down | −0.364435712 | 0.000422283 |
| 59 | 58 | 1 | CDK4 | P11802 | wp160_DB0646_4 hr | DB0646 | down | −0.726257377 | 0.000112674 |
| 59 | 58 | 1 | CDK4 | P11802 | wp160_DB0646_8 hr | DB0646 | down | −1.113878294 | 2.02E−05 |
| 59 | 58 | 1 | CDK4 | P11802 | wp160_SK.3.91_1 hr | SK.3.91 | down | −0.432877345 | 0.000870892 |
| 59 | 58 | 1 | CDK4 | P11802 | wp160_SK.3.91_2 hr | SK.3.91 | down | −0.910378734 | 4.55E−05 |
| 59 | 58 | 1 | CDK4 | P11802 | wp160_SK.3.91_4 hr | SK.3.91 | down | −1.35310233 | 9.21E−06 |
| 59 | 58 | 1 | CDK4 | P11802 | wp168_DB0661 | DB0661 | down | −0.497781626 | 3.38E−05 |
| 59 | 58 | 1 | CDK4 | P11802 | wp168_DB0662 | DB0662 | down | −0.988319856 | 4.71E−07 |
| 59 | 58 | 1 | CDK4 | P11802 | wp168_DB1113 | DB1113 | down | −0.437319263 | 0.000129786 |
| 56 | 53 | 3 | AURKA | O14965 | wp011_TL13.12 | TL13.12 | down | −0.598805318 | 5.58E−05 |
| 56 | 53 | 3 | AURKA | O14965 | wp014_TL13.97 | TL13.97 | down | −0.326824748 | 0.000184259 |
| 56 | 53 | 3 | AURKA | O14965 | wp016_SK.3.91_1 uM | SK.3.91 | down | −3.02404677 | 3.77E−05 |
| 56 | 53 | 3 | AURKA | O14965 | wp016_TL12.186 | TL12.186 | down | −1.758457904 | 0.000381596 |
| 56 | 53 | 3 | AURKA | O14965 | wp016_SK.3.93 | SK.3.93 | down | −1.823112838 | 0.000327742 |
| 56 | 53 | 3 | AURKA | O14965 | wp016_SK.3.91 | SK.3.91 | down | −1.907971669 | 0.00027043 |
| 56 | 53 | 3 | AURKA | O14965 | wp016_SK.3.87 | SK.3.87 | down | −1.381308694 | 0.00104301 |
| 56 | 53 | 3 | AURKA | O14965 | wp016_LT2.49 | LT2.49 | down | −1.853423066 | 0.000305708 |
| 56 | 53 | 3 | AURKA | O14965 | wp016_SK.3.89 | SK.3.89 | down | −1.672156224 | 0.000471407 |
| 56 | 53 | 3 | AURKA | O14965 | wp045_DB0646 | DB0646 | down | −0.324165713 | 0.009613262 |
| 56 | 53 | 3 | AURKA | O14965 | wp045_RSS0628 | RSS0628 | down | −1.130395806 | 5.57E−05 |
| 56 | 53 | 3 | AURKA | O14965 | wp045_DB0614 | DB0614 | down | −0.38504211 | 0.005002499 |
| 56 | 53 | 3 | AURKA | O14965 | wp056_SB1.G.190 | SB1.G.190 | down | −0.842987941 | 0.000543867 |
| 56 | 53 | 3 | AURKA | O14965 | wp071_SB1.G.192.1 | SB1.G.192.1 | down | −0.811107604 | 9.60E−05 |
| 56 | 53 | 3 | AURKA | O14965 | wp071_SB1.G.181 | SB1.G.181 | down | −0.368446495 | 0.003544342 |
| 56 | 53 | 3 | AURKA | O14965 | wp071_JWG.118 | JWG.118 | down | −0.886073378 | 6.29E−05 |
| 56 | 53 | 3 | AURKA | O14965 | wp076_MFH.5.126.1 | MFH.5.126.1 | down | −0.947420971 | 0.000277124 |
| 56 | 53 | 3 | AURKA | O14965 | wp076_MFH.5.116.1 | MFH.5.116.1 | down | −0.706998708 | 0.000957105 |
| 56 | 53 | 3 | AURKA | O14965 | wp076_MFH.5.115.2 | MFH.5.115.2 | down | −0.591217135 | 0.002002414 |
| 56 | 53 | 3 | AURKA | O14965 | wp076_MFH.5.103.1 | MFH.5.103.1 | down | −0.989259628 | 0.00023023 |
| 56 | 53 | 3 | AURKA | O14965 | wp080_dAURK.4 | dAURK.4 | down | −1.055159826 | 4.40E−05 |
| 56 | 53 | 3 | AURKA | O14965 | wp081_SB1.G.192.1 | SB1.G.192.1 | down | −0.729920786 | 3.83E−06 |
| 56 | 53 | 3 | AURKA | O14965 | wp088_SB1.G.194 | SB1.G.194 | down | −1.174760879 | 0.00017375 |
| 56 | 53 | 3 | AURKA | O14965 | wp103_SK.3.91 | SK.3.91 | down | −1.586764171 | 7.94E−07 |
| 56 | 53 | 3 | AURKA | O14965 | wp104_TMX.01.160.1_0.25 | TMX.01.160.1 | down | −1.325895986 | 1.31E−06 |
| 56 | 53 | 3 | AURKA | O14965 | wp104_TMX.01.160.1_1 | TMX.01.160.1 | down | −1.466234208 | 7.85E−07 |
| 56 | 53 | 3 | AURKA | O14965 | wp105_SK.3.91 | SK.3.91 | down | −1.837172963 | 3.58E−08 |
| 56 | 53 | 3 | AURKA | O14965 | wp107_SK.3.91 | SK.3.91 | down | −3.068830234 | 2.92E−08 |
| 56 | 53 | 3 | AURKA | O14965 | wp114_SK.3.91 | SK.3.91 | down | −1.227615743 | 5.42E−05 |
| 56 | 53 | 3 | AURKA | O14965 | wp114_FMF.06.098.1 | FMF.06.098.1 | down | −0.415074482 | 0.003828421 |
| 56 | 53 | 3 | AURKA | O14965 | wp117_DB0614 | DB0614 | down | −0.454739183 | 3.25E−05 |
| 56 | 53 | 3 | AURKA | O14965 | wp131_FMF.06.098.1 | FMF.06.098.1 | down | −0.347149972 | 3.11E−05 |
| 56 | 53 | 3 | AURKA | O14965 | wp131_TL12.186 | TL12.186 | down | −3.192964636 | 1.94E−07 |
| 56 | 53 | 3 | AURKA | O14965 | wp131_SB1.G.192.1 | SB1.G.192.1 | down | −0.626716202 | 1.52E−05 |
| 56 | 53 | 3 | AURKA | O14965 | wp133_SK.3.91_2 hr | SK.3.91 | down | −2.092296522 | 4.85E−10 |
| 56 | 53 | 3 | AURKA | O14965 | wp133_SK.3.91_1 hr | SK.3.91 | down | −1.328541615 | 1.69E−08 |
| 56 | 53 | 3 | AURKA | O14965 | wp133_SK.3.91_4 hr | SK.3.91 | down | −3.744629094 | 3.74E−10 |
| 56 | 53 | 3 | AURKA | O14965 | wp134_JWG.120 | JWG.120 | down | −1.561658165 | 1.30E−07 |
| 56 | 53 | 3 | AURKA | O14965 | wp134_BSJ.04.178 | BSJ.04.178 | down | −0.477238688 | 0.000110447 |
| 56 | 53 | 3 | AURKA | O14965 | wp137_TL12.186 | TL12.186 | down | −2.642168507 | 5.67E−09 |
| 56 | 53 | 3 | AURKA | O14965 | wp137_TL13.97 | TL13.97 | down | −1.611902826 | 8.93E−08 |
| 56 | 53 | 3 | AURKA | O14965 | wp146_TMX.01.160 | TMX.01.160 | down | −2.621191838 | 7.78E−05 |
| 56 | 53 | 3 | AURKA | O14965 | wp146_TMX.02.138 | TMX.02.138 | down | −2.932694944 | 5.17E−05 |
| 56 | 53 | 3 | AURKA | O14965 | wp146_TMX.02.172 | TMX.02.172 | down | −2.401993359 | 0.000106883 |
| 56 | 53 | 3 | AURKA | O14965 | wp146_TMX.02.174 | TMX.02.174 | down | −2.86510208 | 5.63E−05 |
| 56 | 53 | 3 | AURKA | O14965 | wp147_DD.03.106_4 uM | DD.03.106 | down | −0.330229608 | 0.004149118 |
| 56 | 53 | 3 | AURKA | O14965 | wp160_DB0646_4 hr | DB0646 | down | −0.447793356 | 0.007366136 |
| 56 | 53 | 3 | AURKA | O14965 | wp160_SK.3.91_1 hr | SK.3.91 | down | −0.546954361 | 0.003546713 |
| 56 | 53 | 3 | AURKA | O14965 | wp160_SK.3.91_2 hr | SK.3.91 | down | −0.845638295 | 0.000669075 |
| 56 | 53 | 3 | AURKA | O14965 | wp160_SK.3.91_4 hr | SK.3.91 | down | −1.390965928 | 9.31E−05 |
| 56 | 53 | 3 | AURKA | O14965 | wp164_SB1.G.194 | SB1.G.194 | down | −2.034820252 | 6.15E−07 |
| 56 | 53 | 3 | AURKA | O14965 | wp172_SK.3.91 | SK.3.91 | down | −2.047649301 | 3.29E−07 |
| 56 | 53 | 3 | AURKA | O14965 | wp195_BJG.04.203 | BJG.04.203 | down | −0.437568318 | 5.74E−05 |
| 52 | 52 | 0 | WEE1 | P30291-1 | wp005_YKL.6.102 | YKL.6.102 | down | −0.364875879 | 3.43E−05 |
| 52 | 52 | 0 | WEE1 | P30291-1 | wp005_BSJ.01.147 | BSJ.01.147 | down | −0.446967919 | 0.000936431 |
| 52 | 52 | 0 | WEE1 | P30291-1 | wp016_SK.3.91_1 uM | SK.3.91 | down | −2.684017993 | 1.35E−06 |
| 52 | 52 | 0 | WEE1 | P30291-1 | wp016_TL12.186 | TL12.186 | down | −1.514321614 | 1.62E−05 |
| 52 | 52 | 0 | WEE1 | P30291-1 | wp016_SK.3.93 | SK.3.93 | down | −1.176301873 | 4.82E−05 |
| 52 | 52 | 0 | WEE1 | P30291-1 | wp016_SK.3.91 | SK.3.91 | down | −1.277699355 | 3.37E−05 |
| 52 | 52 | 0 | WEE1 | P30291-1 | wp016_SK.3.87 | SK.3.87 | down | −0.927318163 | 0.000133528 |
| 52 | 52 | 0 | WEE1 | P30291-1 | wp016_LT2.49 | LT2.49 | down | −1.314375419 | 2.98E−05 |
| 52 | 52 | 0 | WEE1 | P30291-1 | wp016_SK.3.89 | SK.3.89 | down | −1.017006987 | 9.00E−05 |
| 52 | 52 | 0 | WEE1 | P30291-1 | wp045_DB.3.291 | DB.3.291 | down | −0.416099484 | 0.000338065 |
| 52 | 52 | 0 | WEE1 | P30291-1 | wp045_RSS0628 | RSS0628 | down | −1.578149943 | 1.03E−06 |
| 52 | 52 | 0 | WEE1 | P30291-1 | wp045_DB0614 | DB0614 | down | −1.875908511 | 4.80E−07 |
| 52 | 52 | 0 | WEE1 | P30291-1 | wp056_ZNL.02.096 | ZNL.02.096 | down | −2.409451567 | 1.50E−06 |
| 52 | 52 | 0 | WEE1 | P30291-1 | wp071_SB1.G.192.1 | SB1.G.192.1 | down | −2.366712442 | 1.61E−07 |
| 52 | 52 | 0 | WEE1 | P30291-1 | wp071_JWG.118 | JWG.118 | down | −0.now995351599 | 1.11E−05 |
| 52 | 52 | 0 | WEE1 | P30291-1 | wp076_MFH.5.126.1 | MFH.5.126.1 | down | −0.904762609 | 0.000603386 |
| 52 | 52 | 0 | WEE1 | P30291-1 | wp076_MFH.5.116.1 | MFH.5.116.1 | down | −0.479919786 | 0.00780769 |
| 52 | 52 | 0 | WEE1 | P30291-1 | wp076_MFH.5.115.2 | MFH.5.115.2 | down | −0.766427694 | 0.001208651 |
| 52 | 52 | 0 | WEE1 | P30291-1 | wp076_MFH.5.103.1 | MFH.5.103.1 | down | −0.969378164 | 0.000450501 |
| 52 | 52 | 0 | WEE1 | P30291-1 | wp080_RSS0680 | RSS0680 | down | −2.35874575 | 1.86E−08 |
| 52 | 52 | 0 | WEE1 | P30291-1 | wp081_ZNL.02.096 | ZNL.02.096 | down | −1.970025744 | 8.20E−09 |
| 52 | 52 | 0 | WEE1 | P30291-1 | wp081_SB1.G.192.1 | SB1.G.192.1 | down | −1.764134758 | 1.51E−08 |
| 52 | 52 | 0 | WEE1 | P30291-1 | wp084_BSJ.01.147 | BSJ.01.147 | down | −0.561683696 | 4.38E−06 |
| 52 | 52 | 0 | WEE1 | P30291-1 | wp084_BSJ.03.204 | BSJ.03.204 | down | −0.577358302 | 2.77E−06 |
| 52 | 52 | 0 | WEE1 | P30291-1 | wp084_BSJ.03.123 | BSJ.03.123 | down | −0.388309386 | 1.64E−05 |
| 52 | 52 | 0 | WEE1 | P30291-1 | wp103_SK.3.91 | SK.3.91 | down | −1.37115756 | 2.72E−06 |
| 52 | 52 | 0 | WEE1 | P30291-1 | wp105_SK.3.91 | SK.3.91 | down | −1.228794229 | 4.64E−06 |
| 52 | 52 | 0 | WEE1 | P30291-1 | wp106_ZNL.02.096_4 hr | ZNL.02.096 | down | −2.764223643 | 1.83E−11 |
| 52 | 52 | 0 | WEE1 | P30291-1 | wp106_ZNL.02.096_2 hr | ZNL.02.096 | down | −2.524919377 | 1.06E−12 |
| 52 | 52 | 0 | WEE1 | P30291-1 | wp107_SK.3.91 | SK.3.91 | down | −2.329105721 | 6.52E−10 |
| 52 | 52 | 0 | WEE1 | P30291-1 | wp108_FMF.06.098.1 | FMF.06.098.1 | down | −1.979731649 | 1.41E−06 |
| 52 | 52 | 0 | WEE1 | P30291-1 | wp117_RSS0680 | RSS0680 | down | −1.849987408 | 7.15E−08 |
| 52 | 52 | 0 | WEE1 | P30291-1 | wp117_DB0614 | DB0614 | down | −1.777074052 | 8.06E−09 |
| 52 | 52 | 0 | WEE1 | P30291-1 | wp118_ZNL.02.096 | ZNL.02.096 | down | −2.750381712 | 1.17E−07 |
| 52 | 52 | 0 | WEE1 | P30291-1 | wp131_FMF.06.098.1 | FMF.06.098.1 | down | −2.183426301 | 4.68E−08 |
| 52 | 52 | 0 | WEE1 | P30291-1 | wp131_TL12.186 | TL12.186 | down | −2.57366808 | 8.66E−10 |
| 52 | 52 | 0 | WEE1 | P30291-1 | wp131_SB1.G.192.1 | SB1.G.192.1 | down | −1.64733342 | 1.05E−06 |
| 52 | 52 | 0 | WEE1 | P30291-1 | wp133_SK.3.91_2 hr | SK.3.91 | down | −1.210245509 | 6.78E−09 |
| 52 | 52 | 0 | WEE1 | P30291-1 | wp133_SK.3.91_1 hr | SK.3.91 | down | −0.486577241 | 1.17E−06 |
| 52 | 52 | 0 | WEE1 | P30291-1 | wp133_SK.3.91_4 hr | SK.3.91 | down | −2.314200744 | 7.14E−11 |
| 52 | 52 | 0 | WEE1 | P30291-1 | wp134_BSJ.04.178 | BSJ.04.178 | down | −0.568750558 | 7.04E−05 |
| 52 | 52 | 0 | WEE1 | P30291-1 | wp137_TL12.186 | TL12.186 | down | −2.511235654 | 5.72E−09 |
| 52 | 52 | 0 | WEE1 | P30291-1 | wp137_BSJ.03.204 | BSJ.03.204 | down | −0.675906764 | 8.37E−06 |
| 52 | 52 | 0 | WEE1 | P30291-1 | wp137_TL13.97 | TL13.97 | down | −1.585166074 | 7.45E−08 |
| 52 | 52 | 0 | WEE1 | P30291-1 | wp146_TMX.02.174 | TMX.02.174 | down | −1.14167731 | 0.005358935 |
| 52 | 52 | 0 | WEE1 | P30291-1 | wp160_DB0646_8 hr | DB0646 | down | −0.352030282 | 0.000110766 |
| 52 | 52 | 0 | WEE1 | P30291-1 | wp160_SK.3.91_1 hr | SK.3.91 | down | −0.550264368 | 1.84E−05 |
| 52 | 52 | 0 | WEE1 | P30291-1 | wp160_SK.3.91_2 hr | SK.3.91 | down | −1.20112965 | 7.81E−07 |
| 52 | 52 | 0 | WEE1 | P30291-1 | wp160_SK.3.91_4 hr | SK.3.91 | down | −1.993672709 | 1.00E−07 |
| 52 | 52 | 0 | WEE1 | P30291-1 | wp172_SK.3.91 | SK.3.91 | down | −0.68531798 | 0.000928342 |
| 52 | 52 | 0 | WEE1 | P30291-1 | wp195_BSJ.05.026 | BSJ.05.026 | down | −0.887563052 | 1.36E−07 |
| 52 | 52 | 0 | WEE1 | P30291-1 | wp195_JWG.123 | JWG.123 | down | −1.368473742 | 1.21E−08 |
| 52 | 52 | 0 | BLK | P51451 | wp016_SK.3.91_1 uM | SK.3.91 | down | −1.977117236 | 1.50E−05 |
| 52 | 52 | 0 | BLK | P51451 | wp016_TL12.186 | TL12.186 | down | −1.475387644 | 5.32E−05 |
| 52 | 52 | 0 | BLK | P51451 | wp016_SK.3.93 | SK.3.93 | down | −1.502236754 | 4.92E−05 |
| 52 | 52 | 0 | BLK | P51451 | wp016_SK.3.91 | SK.3.91 | down | −1.445575805 | 5.81E−05 |
| 52 | 52 | 0 | BLK | P51451 | wp016_SK.3.87 | SK.3.87 | down | −0.932857842 | 0.000375345 |
| 52 | 52 | 0 | BLK | P51451 | wp016_LT2.49 | LT2.49 | down | −1.612602125 | 3.63E−05 |
| 52 | 52 | 0 | BLK | P51451 | wp016_SK.3.89 | SK.3.89 | down | −1.335251971 | 8.17E−05 |
| 52 | 52 | 0 | BLK | P51451 | wp045_DB0646 | DB0646 | down | −1.313984666 | 8.86E−06 |
| 52 | 52 | 0 | BLK | P51451 | wp045_DB.3.291 | DB.3.291 | down | −0.585741802 | 0.000293254 |
| 52 | 52 | 0 | BLK | P51451 | wp045_DB0663 | DB0663 | down | −1.057545359 | 2.29E−05 |
| 52 | 52 | 0 | BLK | P51451 | wp047_DD.03.119 | DD.03.119 | down | −1.858918317 | 2.41E−06 |
| 52 | 52 | 0 | BLK | P51451 | wp056_SB1.G.187 | SB1.G.187 | down | −2.020686121 | 5.75E−06 |
| 52 | 52 | 0 | BLK | P51451 | wp071_FMF.05.178.1 | FMF.05.178.1 | down | −0.347915279 | 0.005340908 |
| 52 | 52 | 0 | BLK | P51451 | wp071_SB1.G.192.1 | SB1.G.192.1 | down | −2.398582156 | 6.03E−07 |
| 52 | 52 | 0 | BLK | P51451 | wp071_SB1.G.181 | SB1.G.181 | down | −2.336969269 | 6.85E−07 |
| 52 | 52 | 0 | BLK | P51451 | wp071_SB1.G.200 | SB1.G.200 | down | −1.52490782 | 5.52E−06 |
| 52 | 52 | 0 | BLK | P51451 | wp074_dRAF.3 | dRAF.3 | down | −0.821640968 | 0.001079883 |
| 52 | 52 | 0 | BLK | P51451 | wp081_TL13.150 | TL13.150 | down | −1.434662476 | 1.02E−06 |
| 52 | 52 | 0 | BLK | P51451 | wp081_SB1.G.192.1 | SB1.G.192.1 | down | −2.012815841 | 1.59E−07 |
| 52 | 52 | 0 | BLK | P51451 | wp103_DB0646 | DB0646 | down | −1.189877425 | 1.80E−05 |
| 52 | 52 | 0 | BLK | P51451 | wp103_SK.3.91 | SK.3.91 | down | −1.624810915 | 4.34E−06 |
| 52 | 52 | 0 | BLK | P51451 | wp103_SB1.G.187 | SB1.G.187 | down | −2.09784985 | 1.34E−06 |
| 52 | 52 | 0 | BLK | P51451 | wp107_SK.3.91 | SK.3.91 | down | −1.648714357 | 2.01E−07 |
| 52 | 52 | 0 | BLK | P51451 | wp107_SB1.G.187 | SB1.G.187 | down | −1.916130966 | 1.67E−07 |
| 52 | 52 | 0 | BLK | P51451 | wp107_DB0646 | DB0646 | down | −1.258550283 | 1.82E−06 |
| 52 | 52 | 0 | BLK | P51451 | wp117_DB0646 | DB0646 | down | −1.274932168 | 1.59E−06 |
| 52 | 52 | 0 | BLK | P51451 | wp131_TL12.186 | TL12.186 | down | −2.49717109 | 2.60E−07 |
| 52 | 52 | 0 | BLK | P51451 | wp131_SB1.G.192.1 | SB1.G.192.1 | down | −2.465365866 | 4.77E−08 |
| 52 | 52 | 0 | BLK | P51451 | wp133_SK.3.91_2 hr | SK.3.91 | down | −0.821704365 | 9.73E−08 |
| 52 | 52 | 0 | BLK | P51451 | wp133_SK.3.91_1 hr | SK.3.91 | down | −0.372677546 | 5.36E−06 |
| 52 | 52 | 0 | BLK | P51451 | wp133_SK.3.91_4 hr | SK.3.91 | down | −1.489608078 | 1.18E−09 |
| 52 | 52 | 0 | BLK | P51451 | wp134_DB118430 | DB118430 | down | −0.58261334 | 6.05E−06 |
| 52 | 52 | 0 | BLK | P51451 | wp137_TL12.186 | TL12.186 | down | −2.115445698 | 1.09E−08 |
| 52 | 52 | 0 | BLK | P51451 | wp137_TL13.97 | TL13.97 | down | −2.218742091 | 8.37E−09 |
| 52 | 52 | 0 | BLK | P51451 | wp149_ZNL.03.127 | ZNL.03.127 | down | −0.502448549 | 0.000140877 |
| 52 | 52 | 0 | BLK | P51451 | wp152_DB0646 | DB0646 | down | −1.511440064 | 2.58E−07 |
| 52 | 52 | 0 | BLK | P51451 | wp152_DB1114 | DB1114 | down | −1.312950371 | 5.60E−07 |
| 52 | 52 | 0 | BLK | P51451 | wp152_DB118430 | DB118430 | down | −0.902308886 | 4.38E−06 |
| 52 | 52 | 0 | BLK | P51451 | wp160_DB0646_2 hr | DB0646 | down | −0.53153367 | 0.000100693 |
| 52 | 52 | 0 | BLK | P51451 | wp160_DB0646_4 hr | DB0646 | down | −1.092382435 | 5.53E−06 |
| 52 | 52 | 0 | BLK | P51451 | wp160_DB0646_8 hr | DB0646 | down | −1.434116952 | 1.84E−06 |
| 52 | 52 | 0 | BLK | P51451 | wp160_SK.3.91_2 hr | SK.3.91 | down | −0.87437146 | 1.36E−05 |
| 52 | 52 | 0 | BLK | P51451 | wp160_SK.3.91_4 hr | SK.3.91 | down | −1.67778123 | 9.73E−07 |
| 52 | 52 | 0 | BLK | P51451 | wp168_DB0661 | DB0661 | down | −1.428059814 | 7.33E−08 |
| 52 | 52 | 0 | BLK | P51451 | wp168_DB0662 | DB0662 | down | −1.651569733 | 2.36E−08 |
| 52 | 52 | 0 | BLK | P51451 | wp168_DB1113 | DB1113 | down | −1.320072225 | 1.69E−07 |
| 52 | 52 | 0 | FER | P16591-1 | wp011_TL13.12 | TL13.12 | down | −0.845854571 | 1.07E−06 |
| 52 | 52 | 0 | FER | P16591-1 | wp012_TL13.112 | TL13.112 | down | −0.524748863 | 0.000389042 |
| 52 | 52 | 0 | FER | P16591-1 | wp016_SK.3.91_1 uM | SK.3.91 | down | −3.320727901 | 3.68E−05 |
| 52 | 52 | 0 | FER | P16591-1 | wp016_TL12.186 | TL12.186 | down | −2.727206256 | 8.59E−05 |
| 52 | 52 | 0 | FER | P16591-1 | wp016_SK.3.93 | SK.3.93 | down | −2.640395984 | 9.87E−05 |
| 52 | 52 | 0 | FER | P16591-1 | wp016_SK.3.91 | SK.3.91 | down | −2.645455176 | 9.79E−05 |
| 46 | 46 | 0 | FER | P16591-1 | wp016_SK.3.87 | SK.3.87 | down | −1.969882869 | 0.000342871 |
| 46 | 46 | 0 | FER | P16591-1 | wp016_LT2.49 | LT2.49 | down | −2.466601524 | 0.000132027 |
| 46 | 46 | 0 | FER | P16591-1 | wp016_SK.3.89 | SK.3.89 | down | −2.261998625 | 0.000190929 |
| 46 | 46 | 0 | FER | P16591-1 | wp045_DB0646 | DB0646 | down | −1.711622812 | 3.24E−06 |
| 46 | 46 | 0 | FER | P16591-1 | wp045_DB0663 | DB0663 | down | −0.958054052 | 4.09E−05 |
| 46 | 46 | 0 | FER | P16591-1 | wp045_DB0614 | DB0614 | down | −0.764106561 | 0.000109005 |
| 46 | 46 | 0 | FER | P16591-1 | wp056_SB1.G.187 | SB1.G.187 | down | −1.266634168 | 9.91E−05 |
| 46 | 46 | 0 | FER | P16591-1 | wp081_TL13.150 | TL13.150 | down | −1.511042823 | 1.70E−06 |
| 46 | 46 | 0 | FER | P16591-1 | wp103_DB0646 | DB0646 | down | −1.627643074 | 4.25E−06 |
| 46 | 46 | 0 | FER | P16591-1 | wp103_SK.3.91 | SK.3.91 | down | −2.809916418 | 3.47E−07 |
| 46 | 46 | 0 | FER | P16591-1 | wp103_SB1.G.187 | SB1.G.187 | down | −1.671775761 | 3.76E−06 |
| 46 | 46 | 0 | FER | P16591-1 | wp105_SK.3.91 | SK.3.91 | down | −2.856608568 | 2.73E−09 |
| 46 | 46 | 0 | FER | P16591-1 | wp105_SB1.G.187 | SB1.G.187 | down | −1.43178909 | 3.03E−06 |
| 46 | 46 | 0 | FER | P16591-1 | wp105_DB0646 | DB0646 | down | −0.873745 | 3.15E−06 |
| 46 | 46 | 0 | FER | P16591-1 | wp107_SK.3.91 | SK.3.91 | down | −2.656659888 | 4.04E−09 |
| 46 | 46 | 0 | FER | P16591-1 | wp107_SB1.G.187 | SB1.G.187 | down | −1.759946627 | 6.59E−07 |
| 46 | 46 | 0 | FER | P16591-1 | wp107_DB0646 | DB0646 | down | −1.721797991 | 3.38E−08 |
| 46 | 46 | 0 | FER | P16591-1 | wp108_FMF.06.098.1 | FMF.06.098.1 | down | −0.357703063 | 0.000730248 |
| 46 | 46 | 0 | FER | P16591-1 | wp117_DB0614 | DB0614 | down | −0.772487879 | 5.94E−06 |
| 46 | 46 | 0 | FER | P16591-1 | wp117_DB0646 | DB0646 | down | −1.753147025 | 4.52E−07 |
| 46 | 46 | 0 | FER | P16591-1 | wp131_FMF.06.098.1 | FMF.06.098.1 | down | −0.487066396 | 1.29E−06 |
| 46 | 46 | 0 | FER | P16591-1 | wp131_TL12.186 | TL12.186 | down | −2.582737161 | 5.00E−08 |
| 46 | 46 | 0 | FER | P16591-1 | wp133_SK.3.91_2 hr | SK.3.91 | down | −2.153220466 | 6.66E−07 |
| 46 | 46 | 0 | FER | P16591-1 | wp133_SK.3.91_1 hr | SK.3.91 | down | −1.132131783 | 1.84E−06 |
| 46 | 46 | 0 | FER | P16591-1 | wp133_SK.3.91_4 hr | SK.3.91 | down | −2.426236465 | 1.04E−08 |
| 46 | 46 | 0 | FER | P16591-1 | wp137_TL12.186 | TL12.186 | down | −2.329263776 | 2.33E−07 |
| 46 | 46 | 0 | FER | P16591-1 | wp137_TL13.97 | TL13.97 | down | −2.411625305 | 1.92E−07 |
| 46 | 46 | 0 | FER | P16591-1 | wp146_TMX.02.138 | TMX.02.138 | down | −0.808334843 | 0.000899611 |
| 46 | 46 | 0 | FER | P16591-1 | wp146_TMX.02.174 | TMX.02.174 | down | −1.089122808 | 0.000309005 |
| 46 | 46 | 0 | FER | P16591-1 | wp160_DB0646_2 hr | DB0646 | down | −0.538402353 | 0.001447636 |
| 46 | 46 | 0 | FER | P16591-1 | wp160_DB0646_4 hr | DB0646 | down | −1.454411638 | 2.82E−05 |
| 46 | 46 | 0 | FER | P16591-1 | wp160_DB0646_8 hr | DB0646 | down | −1.737313158 | 1.38E−05 |
| 46 | 46 | 0 | FER | P16591-1 | wp160_SK.3.91_1 hr | SK.3.91 | down | −0.853389153 | 0.000238564 |
| 46 | 46 | 0 | FER | P16591-1 | wp160_SK.3.91_2 hr | SK.3.91 | down | −1.879516108 | 1.00E−05 |
| 46 | 46 | 0 | FER | P16591-1 | wp160_SK.3.91_4 hr | SK.3.91 | down | −1.987317682 | 8.00E−06 |
| 46 | 46 | 0 | FER | P16591-1 | wp168_DB0661 | DB0661 | down | −0.614285682 | 2.58E−05 |
| 46 | 46 | 0 | FER | P16591-1 | wp168_DB0662 | DB0662 | down | −1.480660286 | 1.07E−07 |
| 46 | 46 | 0 | FER | P16591-1 | wp168_DB1113 | DB1113 | down | −1.118493049 | 2.55E−07 |
| 46 | 46 | 0 | CDK6 | Q00534 | wp005_YKL.6.102 | YKL.6.102 | down | −2.452394317 | 1.046−09 |
| 46 | 46 | 0 | CDK6 | Q00534 | wp005_BSJ.01.147 | BSJ.01.147 | down | −2.820308791 | 1.59E−07 |
| 46 | 46 | 0 | CDK6 | Q00534 | wp016_SK.3.91_1 uM | SK.3.91 | down | −1.149154197 | 1.12E−06 |
| 46 | 46 | 0 | CDK6 | Q00534 | wp016_TL12.186 | TL12.186 | down | −0.58312155 | 2.13E−05 |
| 46 | 46 | 0 | CDK6 | Q00534 | wp016_SK.3.93 | SK.3.93 | down | −0.858488947 | 3.98E−06 |
| 46 | 46 | 0 | CDK6 | Q00534 | wp016_SK.3.91 | SK.3.91 | down | −0.883215728 | 3.52E−06 |
| 46 | 46 | 0 | CDK6 | Q00534 | wp016_LT2.49 | LT2.49 | down | −0.653104863 | 1.31E−05 |
| 46 | 46 | 0 | CDK6 | Q00534 | wp016_SK.3.89 | SK.3.89 | down | −0.339649476 | 0.000216797 |
| 46 | 46 | 0 | CDK6 | Q00534 | wp045_RSS0628 | RSS0628 | down | −1.742723404 | 2.16E−08 |
| 46 | 46 | 0 | CDK6 | Q00534 | wp045_DB0614 | DB0614 | down | −2.265087818 | 6.82E−09 |
| 46 | 46 | 0 | CDK6 | Q00534 | wp047_DD.03.106 | DD.03.106 | down | −1.086413863 | 1.11E−07 |
| 46 | 46 | 0 | CDK6 | Q00534 | wp080_RSS0680 | RSS0680 | down | −2.169919634 | 8.34E−09 |
| 46 | 46 | 0 | CDK6 | Q00534 | wp084_BSJ.01.147 | BSJ.01.147 | down | −3.163510879 | 1.53E−08 |
| 46 | 46 | 0 | CDK6 | Q00534 | wp084_BSJ.04.132 | BSJ.04.132 | down | −0.753146658 | 2.58E−06 |
| 46 | 46 | 0 | CDK6 | Q00534 | wp084_BSJ.03.204 | BSJ.03.204 | down | −3.573839639 | 3.05E−10 |
| 46 | 46 | 0 | CDK6 | Q00534 | wp084_BSJ.03.123 | BSJ.03.123 | down | −3.057771506 | 3.62E−10 |
| 46 | 46 | 0 | CDK6 | Q00534 | wp088_SB1.G.194 | SB1.G.194 | down | −0.473099666 | 0.000316149 |
| 46 | 46 | 0 | CDK6 | Q00534 | wp103_SK.3.91 | SK.3.91 | down | −0.934902439 | 8.21E−06 |
| 46 | 46 | 0 | CDK6 | Q00534 | wp103_SB1.G.187 | SB1.G.187 | down | −0.345972559 | 0.000710537 |
| 46 | 46 | 0 | CDK6 | Q00534 | wp104_TMX.01.160.1_0.25 | TMX.01.160.1 | down | −0.865973225 | 6.49E−07 |
| 46 | 46 | 0 | CDK6 | Q00534 | wp104_TMX.01.160.1_1 | TMX.01.160.1 | down | −0.842505246 | 7.47E−07 |
| 46 | 46 | 0 | CDK6 | Q00534 | wp105_SK.3.91 | SK.3.91 | down | −0.542141314 | 0.00011271 |
| 46 | 46 | 0 | CDK6 | Q00534 | wp107_SK.3.91 | SK.3.91 | down | −1.011972618 | 3.16E−07 |
| 46 | 46 | 0 | CDK6 | Q00534 | wp108_FMF.06.098.1 | FMF.06.098.1 | down | −0.358819768 | 0.000171233 |
| 46 | 46 | 0 | CDK6 | Q00534 | wp117_RSS0680 | RSS0680 | down | −2.168651652 | 1.56E−09 |
| 46 | 46 | 0 | CDK6 | Q00534 | wp117_DB0614 | DB0614 | down | −2.079214404 | 9.47E−09 |
| 46 | 46 | 0 | CDK6 | Q00534 | wp131_FMF.06.098.1 | FMF.06.098.1 | down | −0.537735988 | 2.00E−05 |
| 46 | 46 | 0 | CDK6 | Q00534 | wp131_TL12.186 | TL12.186 | down | −0.989237425 | 4.97E−07 |
| 46 | 46 | 0 | CDK6 | Q00534 | wp133_SK.3.91_2 hr | SK.3.91 | down | −0.640360337 | 4.14E−08 |
| 46 | 46 | 0 | CDK6 | Q00534 | wp133_SK.3.91_1 hr | SK.3.91 | down | −0.354242968 | 4.51E−07 |
| 46 | 46 | 0 | CDK6 | Q00534 | wp133_SK.3.91_4 hr | SK.3.91 | down | −1.050219229 | 2.42E−10 |
| 46 | 46 | 0 | CDK6 | Q00534 | wp137_DD.03.106 | DD.03.106 | down | −1.083198519 | 4.75E−09 |
| 46 | 46 | 0 | CDK6 | Q00534 | wp137_TL12.186 | TL12.186 | down | −0.872448872 | 1.59E−08 |
| 46 | 46 | 0 | CDK6 | Q00534 | wp137_BSJ.03.204 | BSJ.03.204 | down | −2.865638872 | 2.07E−11 |
| 46 | 46 | 0 | CDK6 | Q00534 | wp137_TL13.97 | TL13.97 | down | −0.596712648 | 1.32E−07 |
| 46 | 46 | 0 | CDK6 | Q00534 | wp147_DD.03.106_1 uM | DD.03.106 | down | −0.893403704 | 6.54E−07 |
| 46 | 46 | 0 | CDK6 | Q00534 | wp147_DD.03.106_2 uM | DD.03.106 | down | −1.347607828 | 7.88E−08 |
| 46 | 46 | 0 | CDK6 | Q00534 | wp147_DD.03.106_4 uM | DD.03.106 | down | −1.158705761 | 1.72E−07 |
| 46 | 46 | 0 | CDK6 | Q00534 | wp160_SK.3.91_1 hr | SK.3.91 | down | −0.325475694 | 6.58E−05 |
| 46 | 46 | 0 | CDK6 | Q00534 | wp160_SK.3.91_2 hr | SK.3.91 | down | −0.594786083 | 5.79E−06 |
| 46 | 46 | 0 | CDK6 | Q00534 | wp160_SK.3.91_4 hr | SK.3.91 | down | −1.132549371 | 4.26E−07 |
| 46 | 46 | 0 | CDK6 | Q00534 | wp164_SB1.G.194 | SB1.G.194 | down | −1.229699577 | 6.14E−07 |
| 46 | 46 | 0 | GAK | O14976 | wp016_SK.3.91_1 uM | SK.3.91 | down | −0.352623161 | 0.000120929 |
| 46 | 46 | 0 | GAK | O14976 | wp016_TL12.186 | TL12.186 | down | −0.335387672 | 0.000149768 |
| 46 | 46 | 0 | GAK | O14976 | wp016_SK.3.93 | SK.3.93 | down | −0.433395934 | 4.99E−05 |
| 46 | 46 | 0 | GAK | O14976 | wp016_SK.3.91 | SK.3.91 | down | −0.501110401 | 2.67E−05 |
| 46 | 46 | 0 | GAK | O14976 | wp045_DB0646 | DB0646 | down | −0.64236159 | 2.99E−05 |
| 46 | 46 | 0 | GAK | O14976 | wp045_DB0663 | DB0663 | down | −0.690997801 | 2.18E−05 |
| 46 | 46 | 0 | GAK | O14976 | wp045_DB0614 | DB0614 | down | −0.330538382 | 0.000519944 |
| 46 | 46 | 0 | GAK | O14976 | wp056_SB1.G.187 | SB1.G.187 | down | −1.542753923 | 3.76E−06 |
| 46 | 46 | 0 | GAK | O14976 | wp071_SB1.G.192.1 | SB1.G.192.1 | down | −1.76546148 | 1.32E−07 |
| 46 | 46 | 0 | GAK | O14976 | wp071_JWG.118 | JWG.118 | down | −1.972220217 | 7.66E−08 |
| 46 | 46 | 0 | GAK | O14976 | wp080_RSS0680 | RSS0680 | down | −0.777293399 | 1.10E−06 |
| 46 | 46 | 0 | GAK | O14976 | wp081_TL13.150 | TL13.150 | down | −1.014247812 | 1.20E−07 |
| 42 | 42 | 0 | GAK | O14976 | wp081_SB1.G.192.1 | SB1.G.192.1 | down | −1.408635291 | 1.96E−08 |
| 42 | 42 | 0 | GAK | O14976 | wp088_JWG.122 | JWG.122 | down | −0.358568074 | 0.000145671 |
| 42 | 42 | 0 | GAK | O14976 | wp103_DB0646 | DB0646 | down | −0.468436063 | 2.96E−05 |
| 42 | 42 | 0 | GAK | O14976 | wp103_SB1.G.187 | SB1.G.187 | down | −1.478062677 | 1.54E−07 |
| 42 | 42 | 0 | GAK | O14976 | wp105_SK.3.91 | SK.3.91 | down | −0.383210271 | 9.55E−05 |
| 42 | 42 | 0 | GAK | O14976 | wp105_SB1.G.187 | SB1.G.187 | down | −1.088229084 | 3.34E−06 |
| 42 | 42 | 0 | GAK | O14976 | wp105_DB0646 | DB0646 | down | −0.336719993 | 0.000236724 |
| 42 | 42 | 0 | GAK | O14976 | wp107_SB1.G.187 | SB1.G.187 | down | −1.767831584 | 7.55E−09 |
| 42 | 42 | 0 | GAK | O14976 | wp107_DB0646 | DB0646 | down | −0.556356055 | 2.19E−07 |
| 42 | 42 | 0 | GAK | O14976 | wp108_FMF.06.098.1 | FMF.06.098.1 | down | −0.415636 | 0.000412446 |
| 42 | 42 | 0 | GAK | O14976 | wp114_DB0646 | DB0646 | down | −0.33914951 | 0.000161098 |
| 42 | 42 | 0 | GAK | O14976 | wp114_SB1.G.187 | SB1.G.187 | down | −0.357787723 | 0.000130018 |
| 42 | 42 | 0 | GAK | O14976 | wp117_RSS0680 | RSS0680 | down | −0.685277415 | 1.54E−05 |
| 42 | 42 | 0 | GAK | O14976 | wp117_DB0614 | DB0614 | down | −0.419647432 | 0.000262653 |
| 42 | 42 | 0 | GAK | O14976 | wp117_DB0646 | DB0646 | down | −0.586810301 | 5.00E−05 |
| 42 | 42 | 0 | GAK | O14976 | wp131_FMF.06.098.1 | FMF.06.098.1 | down | −0.346906259 | 1.20E−06 |
| 42 | 42 | 0 | GAK | O14976 | wp131_SB1.G.192.1 | SB1.G.192.1 | down | −1.474694791 | 7.71E−10 |
| 42 | 42 | 0 | GAK | O14976 | wp134_BSJ.04.178 | BSJ.04.178 | down | −2.402263747 | 1.80E−10 |
| 42 | 42 | 0 | GAK | O14976 | wp152_DB0646 | DB0646 | down | −0.511745793 | 3.50E−06 |
| 42 | 42 | 0 | GAK | O14976 | wp152_DB1114 | DB1114 | down | −0.999412949 | 8.82E−08 |
| 42 | 42 | 0 | GAK | O14976 | wp160_DB0646_4 hr | DB0646 | down | −0.471378574 | 1.32E−05 |
| 42 | 42 | 0 | GAK | O14976 | wp160_DB0646_8 hr | DB0646 | down | −0.814885534 | 1.44E−06 |
| 42 | 42 | 0 | GAK | O14976 | wp160_SK.3.91_4 hr | SK.3.91 | down | −0.324125749 | 5.95E−05 |
| 42 | 42 | 0 | GAK | O14976 | wp168_DB0662 | DB0662 | down | −0.437312188 | 4.61E−07 |
| 42 | 42 | 0 | GAK | O14976 | wp168_DB1113 | DB1113 | down | −1.028851179 | 1.49E−08 |
| 42 | 42 | 0 | GAK | O14976 | wp172_DB0646 | DB0646 | down | −0.344966104 | 0.000158989 |
| 42 | 42 | 0 | GAK | O14976 | wp172_SB1.G.187 | SB1.G.187 | down | −1.006514 | 2.42E−07 |
| 42 | 42 | 0 | GAK | O14976 | wp195_BSJ.05.026 | BSJ.05.026 | down | −0.581864449 | 8.20E−07 |
| 42 | 42 | 0 | GAK | O14976 | wp195_JWG.123 | JWG.123 | down | −0.524553916 | 1.46E−06 |
| 42 | 42 | 0 | LIMK1 | P53667-1 | wp045_DB0646 | DB0646 | down | −0.529283988 | 0.000446378 |
| 42 | 42 | 0 | LIMK1 | P53667-1 | wp047_DD.03.106 | DD.03.106 | down | −1.63729595 | 0.001113397 |
| 42 | 42 | 0 | LIMK1 | P53667-1 | wp056_DD.03.107 | DD.03.107 | down | −0.959441177 | 0.00131444 |
| 42 | 42 | 0 | LIMK1 | P53667-1 | wp056_DD.03.156 | DD.03.156 | down | −1.036844009 | 0.00096667 |
| 42 | 42 | 0 | LIMK1 | P53667-1 | wp071_SB1.G.181 | SB1.G.181 | down | −0.686036866 | 0.000997449 |
| 42 | 42 | 0 | LIMK1 | P53667-1 | wp071_SB1.G.200 | SB1.G.200 | down | −0.774974297 | 0.000571351 |
| 42 | 42 | 0 | LIMK1 | P53667-1 | wp081_TL13.178 | TL13.178 | down | −0.335947208 | 0.009987053 |
| 42 | 42 | 0 | LIMK1 | P53667-1 | wp103_DB0646 | DB0646 | down | −1.497375017 | 1.40E−05 |
| 42 | 42 | 0 | LIMK1 | P53667-1 | wp103_SK.3.91 | SK.3.91 | down | −1.238400446 | 3.32E−05 |
| 42 | 42 | 0 | LIMK1 | P53667-1 | wp105_SK.3.91 | SK.3.91 | down | −1.463775708 | 8.77E−07 |
| 42 | 42 | 0 | LIMK1 | P53667-1 | wp105_DB0646 | DB0646 | down | −0.564091035 | 0.000209095 |
| 42 | 42 | 0 | LIMK1 | P53667-1 | wp107_SK.3.91 | SK.3.91 | down | −0.870420342 | 1.99E−05 |
| 42 | 42 | 0 | LIMK1 | P53667-1 | wp107_DB0646 | DB0646 | down | −0.97681715 | 3.15E−05 |
| 41 | 41 | 0 | LIMK1 | P53667-1 | wp114_SK.3.91 | SK.3.91 | down | −0.54603649 | 0.002670917 |
| 41 | 41 | 0 | LIMK1 | P53667-1 | wp117_DB0646 | DB0646 | down | −1.412458548 | 5.51E−06 |
| 41 | 41 | 0 | LIMK1 | P53667-1 | wp131_TL12.186 | TL12.186 | down | −1.055417125 | 1.82E−07 |
| 41 | 41 | 0 | LIMK1 | P53667-1 | wp133_SK.3.91_2 hr | SK.3.91 | down | −0.802636012 | 3.90E−06 |
| 41 | 41 | 0 | LIMK1 | P53667-1 | wp133_SK.3.91_1 hr | SK.3.91 | down | −0.411689749 | 0.000240244 |
| 41 | 41 | 0 | LIMK1 | P53667-1 | wp133_SK.3.91_4 hr | SK.3.91 | down | −1.370690745 | 2.51E−07 |
| 41 | 41 | 0 | LIMK1 | P53667-1 | wp137_DD.03.106 | DD.03.106 | down | −1.980601567 | 1.23E−06 |
| 41 | 41 | 0 | LIMK1 | P53667-1 | wp137_TL12.186 | TL12.186 | down | −0.873358209 | 0.00010658 |
| 41 | 41 | 0 | LIMK1 | P53667-1 | wp137_DD.03.107 | DD.03.107 | down | −1.22209666 | 1.74E−05 |
| 41 | 41 | 0 | LIMK1 | P53667-1 | wp137_TL13.97 | TL13.97 | down | −1.17329608 | 2.18E−05 |
| 41 | 41 | 0 | LIMK1 | P53667-1 | wp147_DD.03.106_1 uM | DD.03.106 | down | −2.959035081 | 1.57E−07 |
| 41 | 41 | 0 | LIMK1 | P53667-1 | wp147_DD.03.106_2 uM | DD.03.106 | down | −2.199776311 | 7.22E−07 |
| 41 | 41 | 0 | LIMK1 | P53667-1 | wp147_DD.03.106_4 uM | DD.03.106 | down | −0.637017148 | 0.000375166 |
| 41 | 41 | 0 | LIMK1 | P53667-1 | wp147_DD.03.107_1 uM | DD.03.107 | down | −0.969127195 | 4.71E−05 |
| 41 | 41 | 0 | LIMK1 | P53667-1 | wp147_DD.03.107_2 uM | DD.03.107 | down | −1.375452547 | 7.99E−06 |
| 41 | 41 | 0 | LIMK1 | P53667-1 | wp147_DD.03.107_4 uM | DD.03.107 | down | −1.536467117 | 4.54E−06 |
| 41 | 41 | 0 | LIMK1 | P53667-1 | wp149_ZNL.03.127 | ZNL.03.127 | down | −0.476376643 | 0.001388336 |
| 41 | 41 | 0 | LIMK1 | P53667-1 | wp160_DB0646_2 hr | DB0646 | down | −0.737568964 | 0.003760906 |
| 41 | 41 | 0 | LIMK1 | P53667-1 | wp160_DB0646_4 hr | DB0646 | down | −1.562905074 | 0.000205254 |
| 41 | 41 | 0 | LIMK1 | P53667-1 | wp160_DB0646_8 hr | DB0646 | down | −2.359561283 | 3.95E−05 |
| 41 | 41 | 0 | LIMK1 | P53667-1 | wp160_SK.3.91_2 hr | SK.3.91 | down | −0.863399204 | 0.002080848 |
| 41 | 41 | 0 | LIMK1 | P53667-1 | wp160_SK.3.91_4 hr | SK.3.91 | down | −1.365773727 | 0.000350268 |
| 41 | 41 | 0 | LIMK1 | P53667-1 | wp168_DB0661 | DB0661 | down | −1.115823405 | 2.39E−05 |
| 41 | 41 | 0 | LIMK1 | P53667-1 | wp168_DB0662 | DB0662 | down | −1.910558529 | 3.04E−06 |
| 41 | 41 | 0 | LIMK1 | P53667-1 | wp168_DB1113 | DB1113 | down | −1.144116634 | 2.09E−05 |
| 41 | 41 | 0 | LIMK1 | P53667-1 | wp172_DB0646 | DB0646 | down | −1.319837835 | 1.63E−05 |
| 41 | 41 | 0 | LIMK1 | P53667-1 | wp172_SK.3.91 | SK.3.91 | down | −1.868800692 | 1.39E−06 |
| 41 | 41 | 0 | LIMK2 | P53671 | wp016_SK.3.91_1 uM | SK.3.91 | down | −1.441231961 | 1.44E−05 |
| 41 | 41 | 0 | LIMK2 | P53671 | wp016_TL12.186 | TL12.186 | down | −0.739539982 | 0.000252654 |
| 41 | 41 | 0 | LIMK2 | P53671 | wp016_SK.3.93 | SK.3.93 | down | −1.009573613 | 6.69E−05 |
| 41 | 41 | 0 | LIMK2 | P53671 | wp016_SK.3.91 | SK.3.91 | down | −1.061078669 | 5.40E−05 |
| 41 | 41 | 0 | LIMK2 | P53671 | wp016_SK.3.87 | SK.3.87 | down | −0.377968628 | 0.003965745 |
| 41 | 41 | 0 | LIMK2 | P53671 | wp016_LT2.49 | LT2.49 | down | −0.957274758 | 8.41E−05 |
| 41 | 41 | 0 | LIMK2 | P53671 | wp016_SK.3.89 | SK.3.89 | down | −0.444014948 | 0.002093287 |
| 41 | 41 | 0 | LIMK2 | P53671 | wp045_DB0646 | DB0646 | down | −1.647941747 | 3.07E−06 |
| 41 | 41 | 0 | LIMK2 | P53671 | wp045_DB0663 | DB0663 | down | −1.366587978 | 6.97E−06 |
| 41 | 41 | 0 | LIMK2 | P53671 | wp045_RSS0628 | RSS0628 | down | −0.718238128 | 0.000114512 |
| 41 | 41 | 0 | LIMK2 | P53671 | wp045_DB0614 | DB0614 | down | −0.436988383 | 0.000943526 |
| 41 | 41 | 0 | LIMK2 | P53671 | wp056_DD.03.156 | DD.03.156 | down | −1.702866946 | 9.72E−06 |
| 41 | 41 | 0 | LIMK2 | P53671 | wp074_dRAF.3 | dRAF.3 | down | −0.465646949 | 0.003400078 |
| 41 | 41 | 0 | LIMK2 | P53671 | wp080_RSS0680 | RSS0680 | down | −0.880894782 | 0.004255828 |
| 41 | 40 | 1 | LIMK2 | P53671 | wp081_SB1.G.192.1 | SB1.G.192.1 | down | −0.371017656 | 0.00011483 |
| 41 | 40 | 1 | LIMK2 | P53671 | wp103_DB0646 | DB0646 | down | −1.568775251 | 4.16E−05 |
| 41 | 40 | 1 | LIMK2 | P53671 | wp103_SK.3.91 | SK.3.91 | down | −1.430713809 | 6.31E−05 |
| 41 | 40 | 1 | LIMK2 | P53671 | wp103_WH.10417.099 | WH.10417.099 | down | −0.574686499 | 0.00335619 |
| 41 | 40 | 1 | LIMK2 | P53671 | wp107_SK.3.91 | SK.3.91 | down | −1.87814574 | 0.000249119 |
| 41 | 40 | 1 | LIMK2 | P53671 | wp107_DB0646 | DB0646 | down | −1.738377486 | 2.18E−06 |
| 41 | 40 | 1 | LIMK2 | P53671 | wp107_WH.10417.099 | WH.10417.099 | down | −0.374430831 | 0.005837852 |
| 41 | 40 | 1 | LIMK2 | P53671 | wp108_FMF.06.098.1 | FMF.06.098.1 | down | −0.82259334 | 6.19E−06 |
| 41 | 40 | 1 | LIMK2 | P53671 | wp114_SK.3.91 | SK.3.91 | down | −0.841364651 | 0.005516366 |
| 41 | 40 | 1 | LIMK2 | P53671 | wp114_FMF.06.098.1 | FMF.06.098.1 | down | −1.629208028 | 0.000438481 |
| 41 | 40 | 1 | LIMK2 | P53671 | wp117_RSS0680 | RSS0680 | down | −1.109015956 | 2.85E−05 |
| 41 | 40 | 1 | LIMK2 | P53671 | wp117_DB0646 | DB0646 | down | −1.564708834 | 8.77E−06 |
| 41 | 40 | 1 | LIMK2 | P53671 | wp131_FMF.06.098.1 | FMF.06.098.1 | down | −1.076913753 | 3.59E−08 |
| 41 | 40 | 1 | LIMK2 | P53671 | wp131_TL12.186 | TL12.186 | down | −1.322561578 | 2.84E−07 |
| 41 | 40 | 1 | LIMK2 | P53671 | wp131_SB1.G.192.1 | SB1.G.192.1 | down | −0.5543586 | 0.001044327 |
| 41 | 40 | 1 | LIMK2 | P53671 | wp133_SK.3.91_2 hr | SK.3.91 | down | −0.757517421 | 7.96E−08 |
| 41 | 40 | 1 | LIMK2 | P53671 | wp133_SK.3.91_4 hr | SK.3.91 | down | −1.341898292 | 2.47E−09 |
| 41 | 40 | 1 | LIMK2 | P53671 | wp137_DD.03.106 | DD.03.106 | down | −1.026048282 | 7.79E−07 |
| 41 | 40 | 1 | LIMK2 | P53671 | wp137_TL12.186 | TL12.186 | down | −1.179157356 | 3.60E−07 |
| 41 | 40 | 1 | LIMK2 | P53671 | wp137_TL13.97 | TL13.97 | down | −0.905388184 | 1.56E−06 |
| 41 | 40 | 1 | LIMK2 | P53671 | wp149_ZNL.03.127 | ZNL.03.127 | down | −0.394441546 | 0.000153486 |
| 41 | 40 | 1 | LIMK2 | P53671 | wp152_DB0646 | DB0646 | down | −1.564989828 | 1.97E−08 |
| 41 | 40 | 1 | LIMK2 | P53671 | wp152_DB1114 | DB1114 | down | −1.621241757 | 1.62E−08 |
| 41 | 40 | 1 | LIMK2 | P53671 | wp152_DB118430 | DB118430 | down | −0.457062765 | 1.69E−05 |
| 41 | 40 | 1 | LIMK2 | P53671 | wp172_DB0646 | DB0646 | down | −1.094764193 | 0.000633241 |
| 41 | 40 | 1 | LIMK2 | P53671 | wp172_SK.3.91 | SK.3.91 | down | −0.898425598 | 6.84E−05 |
| 41 | 40 | 1 | AAK1 | Q2M218-1 | wp016_SK.3.91_1 uM | SK.3.91 | down | −1.113850155 | 1.19E−05 |
| 41 | 40 | 1 | AAK1 | Q2M218-1 | wp016_TL12.186 | TL12.186 | down | −1.337954542 | 5.36E−06 |
| 41 | 40 | 1 | AAK1 | Q2M218-1 | wp016_SK.3.93 | SK.3.93 | down | −1.58828042 | 2.54E−06 |
| 41 | 40 | 1 | AAK1 | Q2M218-1 | wp016_SK.3.91 | SK.3.91 | down | −1.363527962 | 4.94E−06 |
| 41 | 40 | 1 | AAK1 | Q2M218-1 | wp016_SK.3.87 | SK.3.87 | down | −0.730694503 | 7.33E−05 |
| 41 | 40 | 1 | AAK1 | Q2M218-1 | wp016_LT2.49 | LT2.49 | down | −1.777827603 | 1.56E−06 |
| 41 | 40 | 1 | AAK1 | Q2M218-1 | wp016_SK.3.89 | SK.3.89 | down | −1.219394212 | 8.02E−06 |
| 41 | 40 | 1 | AAK1 | Q2M218-1 | wp045_RSS0628 | RSS0628 | down | −1.240067133 | 1.87E−06 |
| 41 | 40 | 1 | AAK1 | Q2M218-1 | wp045_DB0614 | DB0614 | down | −1.862805646 | 3.13E−07 |
| 41 | 40 | 1 | AAK1 | Q2M218-1 | wp056_SB1.G.190 | SB1.G.190 | down | −1.489117636 | 4.30E−06 |
| 41 | 40 | 1 | AAK1 | Q2M218-1 | wp071_JWG.118 | JWG.118 | down | −3.044360715 | 8.95E−09 |
| 41 | 40 | 1 | AAK1 | Q2M218-1 | wp080_RSS0680 | RSS0680 | down | −1.392112838 | 2.54E−07 |
| 41 | 40 | 1 | AAK1 | Q2M218-1 | wp088_JWG.122 | JWG.122 | down | −0.633532135 | 3.87E−06 |
| 41 | 40 | 1 | AAK1 | Q2M218-1 | wp088_SB1.G.194 | SB1.G.194 | down | −0.348201537 | 6.98E−05 |
| 39 | 39 | 0 | AAK1 | Q2M218-1 | wp103_SK.3.91 | SK.3.91 | down | −0.776283951 | 5.50E−05 |
| 39 | 39 | 0 | AAK1 | Q2M218-1 | wp105_SK.3.91 | SK.3.91 | down | −1.574546975 | 2.50E−09 |
| 39 | 39 | 0 | AAK1 | Q2M218-1 | wp107_SK.3.91 | SK.3.91 | down | −1.058875581 | 7.28E−08 |
| 39 | 39 | 0 | AAK1 | Q2M218-1 | wp108_FMF.06.098.1 | FMF.06.098.1 | down | −0.434596049 | 0.000435933 |
| 39 | 39 | 0 | AAK1 | Q2M218-1 | wp114_SK.3.91 | SK.3.91 | down | −0.822076041 | 0.000116154 |
| 39 | 39 | 0 | AAK1 | Q2M218-1 | wp114_FMF.06.098.1 | FMF.06.098.1 | down | −0.484978488 | 0.00094162 |
| 39 | 39 | 0 | AAK1 | Q2M218-1 | wp117_RSS0680 | RSS0680 | down | −1.050132492 | 1.24E−07 |
| 39 | 39 | 0 | AAK1 | Q2M218-1 | wp117_DB0614 | DB0614 | down | −1.652144917 | 6.66E−08 |
| 39 | 39 | 0 | AAK1 | Q2M218-1 | wp131_FMF.06.098.1 | FMF.06.098.1 | down | −0.729330466 | 9.25E−07 |
| 39 | 39 | 0 | AAK1 | Q2M218-1 | wp131_TL12.186 | TL12.186 | down | −1.519996088 | 3.80E−08 |
| 39 | 39 | 0 | AAK1 | Q2M218-1 | wp133_SK.3.91_2 hr | SK.3.91 | down | −0.550561422 | 2.87E−08 |
| 39 | 39 | 0 | AAK1 | Q2M218-1 | wp133_SK.3.91_4 hr | SK.3.91 | down | −1.104789094 | 1.44E−09 |
| 39 | 39 | 0 | AAK1 | Q2M218-1 | wp134_JWG.120 | JWG.120 | down | −0.33622525 | 0.000578906 |
| 39 | 39 | 0 | AAK1 | Q2M218-1 | wp134_BSJ.04.178 | BSJ.04.178 | down | −3.341722858 | 1.23E−09 |
| 39 | 39 | 0 | AAK1 | Q2M218-1 | wp137_TL12.186 | TL12.186 | down | −1.309938959 | 5.26E−09 |
| 39 | 39 | 0 | AAK1 | Q2M218-1 | wp137_TL13.97 | TL13.97 | down | −2.478578498 | 1.49E−10 |
| 39 | 39 | 0 | AAK1 | Q2M218-1 | wp146_TMX.02.138 | TMX.02.138 | down | −1.129340953 | 9.17E−05 |
| 39 | 39 | 0 | AAK1 | Q2M218-1 | wp146_TMX.02.174 | TMX.02.174 | down | −1.619781842 | 2.46E−05 |
| 39 | 39 | 0 | AAK1 | Q2M218-1 | wp160_SK.3.91_2 hr | SK.3.91 | down | −0.515257493 | 3.77E−05 |
| 39 | 39 | 0 | AAK1 | Q2M218-1 | wp160_SK.3.91_4 hr | SK.3.91 | down | −1.007775889 | 2.51E−06 |
| 39 | 39 | 0 | AAK1 | Q2M218-1 | wp164_SB1.G.194 | SB1.G.194 | down | −0.378990399 | 1.76E−05 |
| 39 | 39 | 0 | AAK1 | Q2M218-1 | wp172_SK.3.91 | SK.3.91 | down | −0.975458458 | 8.28E−08 |
| 39 | 39 | 0 | AAK1 | Q2M218-1 | wp195_BJG.04.203 | BJG.04.203 | down | −1.336013296 | 1.06E−08 |
| 39 | 39 | 0 | AAK1 | Q2M218-1 | wp195_BSJ.05.026 | BSJ.05.026 | down | −3.073212154 | 1.00E−10 |
| 39 | 39 | 0 | AAK1 | Q2M218-1 | wp195_JWG.123 | JWG.123 | down | −1.091137149 | 3.29E−08 |
| 39 | 39 | 0 | CSK | P41240 | wp016_SK.3.91_1 uM | SK.3.91 | down | −0.659563474 | 4.54E−05 |
| 39 | 39 | 0 | CSK | P41240 | wp016_SK.3.91 | SK.3.91 | down | −0.325391057 | 0.000906379 |
| 39 | 39 | 0 | CSK | P41240 | wp045_DB0646 | DB0646 | down | −1.541711976 | 5.55E−08 |
| 39 | 39 | 0 | CSK | P41240 | wp045_DB.3.291 | DB.3.291 | down | −2.256711456 | 1.04E−08 |
| 39 | 39 | 0 | CSK | P41240 | wp045_DB0663 | DB0663 | down | −1.206133445 | 1.64E−07 |
| 39 | 39 | 0 | CSK | P41240 | wp047_DD.03.119 | DD.03.119 | down | −1.786148027 | 4.51E−08 |
| 39 | 39 | 0 | CSK | P41240 | wp071_FMF.05.178.1 | FMF.05.178.1 | down | −0.477901096 | 5.94E−05 |
| 39 | 39 | 0 | CSK | P41240 | wp071_SB1.G.192.1 | SB1.G.192.1 | down | −2.271344792 | 2.95E−08 |
| 39 | 39 | 0 | CSK | P41240 | wp071_SB1.G.181 | SB1.G.181 | down | −3.229388644 | 5.24E−09 |
| 39 | 39 | 0 | CSK | P41240 | wp071_SB1.G.200 | SB1.G.200 | down | −3.011057511 | 7.39E−09 |
| 39 | 39 | 0 | CSK | P41240 | wp074_dRAF.3 | dRAF.3 | down | −2.093658305 | 3.48E−07 |
| 39 | 39 | 0 | CSK | P41240 | wp081_SB1.G.192.1 | SB1.G.192.1 | down | −1.965866254 | 4.36E−10 |
| 39 | 39 | 0 | CSK | P41240 | wp103_DB0646 | DB0646 | down | −1.281877209 | 8.40E−07 |
| 39 | 39 | 0 | CSK | P41240 | wp103_SK.3.91 | SK.3.91 | down | −0.434292658 | 0.000116225 |
| 39 | 39 | 0 | CSK | P41240 | wp105_SK.3.91 | SK.3.91 | down | −0.704365023 | 7.51E−07 |
| 39 | 39 | 0 | CSK | P41240 | wp105_DB0646 | DB0646 | down | −0.686772492 | 9.90E−07 |
| 39 | 39 | 0 | CSK | P41240 | wp107_SK.3.91 | SK.3.91 | down | −0.5646008 | 1.18E−06 |
| 39 | 39 | 0 | CSK | P41240 | wp107_DB0646 | DB0646 | down | −1.311237076 | 5.67E−08 |
| 39 | 39 | 0 | CSK | P41240 | wp114_DB0646 | DB0646 | down | −0.693150505 | 3.21E−05 |
| 39 | 39 | 0 | CSK | P41240 | wp117_DB0646 | DB0646 | down | −1.413710962 | 6.97E−08 |
| 39 | 39 | 0 | CSK | P41240 | wp131_SB1.G.192.1 | SB1.G.192.1 | down | −1.816031982 | 1.61E−09 |
| 39 | 39 | 0 | CSK | P41240 | wp133_SK.3.91_4 hr | SK.3.91 | down | −0.549176499 | 2.12E−07 |
| 39 | 39 | 0 | CSK | P41240 | wp147_DD.03.106_4 uM | DD.03.106 | down | −0.51493628 | 6.82E−06 |
| 39 | 39 | 0 | CSK | P41240 | wp152_DB0646 | DB0646 | down | −1.513035307 | 1.80E−09 |
| 39 | 39 | 0 | CSK | P41240 | wp152_DB1114 | DB1114 | down | −1.520569538 | 1.75E−09 |
| 39 | 39 | 0 | CSK | P41240 | wp160_DB0646_2 hr | DB0646 | down | −0.830769775 | 1.38E−05 |
| 39 | 39 | 0 | CSK | P41240 | wp160_DB0646_4 hr | DB0646 | down | −1.416410255 | 1.59E−06 |
| 39 | 39 | 0 | CSK | P41240 | wp160_DB0646_8 hr | DB0646 | down | −1.752845509 | 6.70E−07 |
| 39 | 39 | 0 | CSK | P41240 | wp160_SK.3.91_4 hr | SK.3.91 | down | −0.49764646 | 0.000108042 |
| 39 | 39 | 0 | CSK | P41240 | wp168_DB0661 | DB0661 | down | −1.526099631 | 9.72E−08 |
| 39 | 39 | 0 | CSK | P41240 | wp168_DB0662 | DB0662 | down | −1.832861235 | 2.86E−08 |
| 39 | 39 | 0 | CSK | P41240 | wp168_DB1113 | DB1113 | down | −1.619991717 | 1.14E−07 |
| 39 | 39 | 0 | CSK | P41240 | wp172_DB0646 | DB0646 | down | −0.853812085 | 4.56E−07 |
| 39 | 39 | 0 | CSK | P41240 | wp172_SK.3.91 | SK.3.91 | down | −0.422482654 | 8.89E−06 |
| 39 | 39 | 0 | SIK2 | Q9H0K1 | wp045_DB0646 | DB0646 | down | −0.638036925 | 0.00229682 |
| 39 | 39 | 0 | SIK2 | Q9H0K1 | wp045_DB0663 | DB0663 | down | −0.476521872 | 0.007211898 |
| 39 | 39 | 0 | SIK2 | Q9H0K1 | wp045_DB0614 | DB0614 | down | −0.529168062 | 0.00482292 |
| 39 | 39 | 0 | SIK2 | Q9H0K1 | wp071_SB1.G.192.1 | SB1.G.192.1 | down | −1.036439592 | 0.001154912 |
| 39 | 39 | 0 | SIK2 | Q9H0K1 | wp080_RSS0680 | RSS0680 | down | −0.698615584 | 0.0002511 |
| 39 | 39 | 0 | SIK2 | Q9H0K1 | wp081_SB1.G.192.1 | SB1.G.192.1 | down | −1.195541788 | 0.00075419 |
| 39 | 39 | 0 | SIK2 | Q9H0K1 | wp105_SK.3.91 | SK.3.91 | down | −0.538614024 | 0.000423483 |
| 39 | 39 | 0 | SIK2 | Q9H0K1 | wp107_SK.3.91 | SK.3.91 | down | −1.568083896 | 4.80E−05 |
| 39 | 39 | 0 | SIK2 | Q9H0K1 | wp107_DB0646 | DB0646 | down | −0.593269516 | 0.003400477 |
| 39 | 39 | 0 | SIK2 | Q9H0K1 | wp114_DB0646 | DB0646 | down | −0.434673573 | 0.004107626 |
| 39 | 39 | 0 | SIK2 | Q9H0K1 | wp114_SK.3.91 | SK.3.91 | down | −0.575650629 | 0.001416507 |
| 39 | 39 | 0 | SIK2 | Q9H0K1 | wp114_FMF.06.098.1 | FMF.06.098.1 | down | −0.498470908 | 0.002457167 |
| 39 | 39 | 0 | SIK2 | Q9H0K1 | wp117_RSS0680 | RSS0680 | down | −0.7184217 | 7.28E−05 |
| 39 | 39 | 0 | SIK2 | Q9H0K1 | wp117_DB0614 | DB0614 | down | −0.376552221 | 0.000663689 |
| 39 | 39 | 0 | SIK2 | Q9H0K1 | wp117_DB0646 | DB0646 | down | −0.431511791 | 0.00042948 |
| 39 | 39 | 0 | SIK2 | Q9H0K1 | wp131_FMF.06.098.1 | FMF.06.098.1 | down | −0.602020622 | 2.09E−05 |
| 39 | 39 | 0 | SIK2 | Q9H0K1 | wp131_TL12.186 | TL12.186 | down | −1.316931454 | 2.75E−07 |
| 39 | 39 | 0 | SIK2 | Q9H0K1 | wp131_SB1.G.192.1 | SB1.G.192.1 | down | −0.934168194 | 2.90E−06 |
| 39 | 39 | 0 | SIK2 | Q9H0K1 | wp133_SK.3.91_2 hr | SK.3.91 | down | −0.493805829 | 2.09E−05 |
| 37 | 36 | 1 | SIK2 | Q9H0K1 | wp133_SK.3.91_4 hr | SK.3.91 | down | −1.034518399 | 2.74E−07 |
| 37 | 36 | 1 | SIK2 | Q9H0K1 | wp134_DB118430 | DB118430 | down | −0.380289621 | 0.001038171 |
| 37 | 36 | 1 | SIK2 | Q9H0K1 | wp134_BSJ.04.178 | BSJ.04.178 | down | −1.351122265 | 9.15E−07 |
| 37 | 36 | 1 | SIK2 | Q9H0K1 | wp152_DB1114 | DB1114 | down | −0.542484866 | 0.005438132 |
| 37 | 36 | 1 | SIK2 | Q9H0K1 | wp160_DB0646_4 hr | DB0646 | down | −0.542100323 | 0.003312508 |
| 37 | 36 | 1 | SIK2 | Q9H0K1 | wp160_DB0646_8 hr | DB0646 | down | −0.570729654 | 0.002731432 |
| 37 | 36 | 1 | SIK2 | Q9H0K1 | wp160_SK.3.91_2 hr | SK.3.91 | down | −0.54126933 | 0.00333153 |
| 37 | 36 | 1 | SIK2 | Q9H0K1 | wp160_SK.3.91_4 hr | SK.3.91 | down | −0.836394333 | 0.000627859 |
| 37 | 36 | 1 | SIK2 | Q9H0K1 | wp168_DB0661 | DB0661 | down | −0.362847274 | 0.000792078 |
| 37 | 36 | 1 | SIK2 | Q9H0K1 | wp168_DB0662 | DB0662 | down | −0.776995951 | 1.45E−05 |
| 37 | 36 | 1 | SIK2 | Q9H0K1 | wp168_DB1113 | DB1113 | down | −0.729582699 | 2.32E−05 |
| 37 | 36 | 1 | SIK2 | Q9H0K1 | wp195_BSJ.05.026 | BSJ.05.026 | down | −0.94719792 | 1.78E−06 |
| 37 | 36 | 1 | SIK2 | Q9H0K1 | wp195_JWG.123 | JWG.123 | down | −0.614269723 | 1.93E−05 |
| 37 | 36 | 1 | SIK2 | Q9H0K1 | wp195_JWG.148 | JWG.148 | down | −0.454965511 | 9.75E−05 |
| 37 | 36 | 1 | CDK2 | P24941-1 | wp016_SK.3.91_1 uM | SK.3.91 | down | −1.047024163 | 2.06E−05 |
| 37 | 36 | 1 | CDK2 | P24941-1 | wp016_TL12.186 | TL12.186 | down | −0.597063821 | 0.000228963 |
| 37 | 36 | 1 | CDK2 | P24941-1 | wp016_SK.3.93 | SK.3.93 | down | −0.543301049 | 0.00034124 |
| 37 | 36 | 1 | CDK2 | P24941-1 | wp016_SK.3.91 | SK.3.91 | down | −0.51148242 | 0.000439863 |
| 37 | 36 | 1 | CDK2 | P24941-1 | wp016_SK.3.87 | SK.3.87 | down | −0.379683443 | 0.001509966 |
| 37 | 36 | 1 | CDK2 | P24941-1 | wp016_LT2.49 | LT2.49 | down | −0.517689644 | 0.000418137 |
| 37 | 36 | 1 | CDK2 | P24941-1 | wp016_SK.3.89 | SK.3.89 | down | −0.595307629 | 0.000231841 |
| 37 | 36 | 1 | CDK2 | P24941-1 | wp080_RSS0680 | RSS0680 | down | −0.420173952 | 0.000116581 |
| 37 | 36 | 1 | CDK2 | P24941-1 | wp103_SK.3.91 | SK.3.91 | down | −0.458753156 | 0.000280755 |
| 37 | 36 | 1 | CDK2 | P24941-1 | wp104_TMX.01.160.1_0.25 | TMX.01.160.1 | down | −1.023915789 | 2.42E−05 |
| 37 | 36 | 1 | CDK2 | P24941-1 | wp104_TMX.01.160.1_1 | TMX.01.160.1 | down | −0.945708158 | 3.61E−05 |
| 37 | 36 | 1 | CDK2 | P24941-1 | wp105_SK.3.91 | SK.3.91 | down | −0.459289152 | 1.21E−05 |
| 37 | 36 | 1 | CDK2 | P24941-1 | wp107_SK.3.91 | SK.3.91 | down | −0.758340735 | 2.40E−06 |
| 37 | 36 | 1 | CDK2 | P24941-1 | wp108_FMF.06.098.1 | FMF.06.098.1 | down | −0.650084287 | 2.88E−05 |
| 37 | 36 | 1 | CDK2 | P24941-1 | wp117_RSS0680 | RSS0680 | down | −0.658846828 | 0.000185367 |
| 37 | 36 | 1 | CDK2 | P24941-1 | wp131_FMF.06.098.1 | FMF.06.098.1 | down | −0.872500979 | 6.73E−06 |
| 37 | 36 | 1 | CDK2 | P24941-1 | wp131_TL12.186 | TL12.186 | down | −0.979964074 | 1.51E−06 |
| 37 | 36 | 1 | CDK2 | P24941-1 | wp133_SK.3.91_2 hr | SK.3.91 | down | −0.430575964 | 8.68E−07 |
| 37 | 36 | 1 | CDK2 | P24941-1 | wp133_SK.3.91_4 hr | SK.3.91 | down | −0.690807724 | 4.00E−07 |
| 37 | 36 | 1 | CDK2 | P24941-1 | wp137_DD.03.106 | DD.03.106 | down | −0.343725549 | 0.000128338 |
| 37 | 36 | 1 | CDK2 | P24941-1 | wp137_TL12.186 | TL12.186 | down | −0.916158676 | 6.08E−07 |
| 37 | 36 | 1 | CDK2 | P24941-1 | wp137_TL13.97 | TL13.97 | down | −0.783372072 | 1.45E−06 |
| 34 | 34 | 0 | CDK2 | P24941-1 | wp146_TMX.01.160 | TMX.01.160 | down | −1.970790057 | 0.000190135 |
| 34 | 34 | 0 | CDK2 | P24941-1 | wp146_TMX.02.138 | TMX.02.138 | down | −2.269637395 | 0.00011395 |
| 34 | 34 | 0 | CDK2 | P24941-1 | wp146_TMX.02.172 | TMX.02.172 | down | −2.199028271 | 0.000127805 |
| 34 | 34 | 0 | CDK2 | P24941-1 | wp146_TMX.02.174 | TMX.02.174 | down | −2.295173916 | 0.000109412 |
| 34 | 34 | 0 | CDK2 | P24941-1 | wp147_DD.03.106_1 uM | DD.03.106 | down | −0.328549573 | 7.48E−06 |
| 34 | 34 | 0 | CDK2 | P24941-1 | wp147_DD.03.106_2 uM | DD.03.106 | down | −0.49318596 | 9.36E−07 |
| 34 | 34 | 0 | CDK2 | P24941-1 | wp147_DD.03.106_4 uM | DD.03.106 | down | −0.52480849 | 6.80E−07 |
| 34 | 34 | 0 | CDK2 | P24941-1 | wp160_SK.3.91_2 hr | SK.3.91 | down | −0.38302149 | 4.24E−05 |
| 34 | 34 | 0 | CDK2 | P24941-1 | wp160_SK.3.91_4 hr | SK.3.91 | down | −0.647029463 | 5.11E−06 |
| 34 | 34 | 0 | CDK2 | P24941-1 | wp172_SK.3.91 | SK.3.91 | down | −0.899596993 | 6.62E−07 |
| 34 | 34 | 0 | CDK5 | Q00535 | wp016_SK.3.91_1 uM | SK.3.91 | down | −1.428776058 | 1.63E−06 |
| 34 | 34 | 0 | CDK5 | Q00535 | wp016_TL12.186 | TL12.186 | down | −0.926081968 | 1.08E−05 |
| 34 | 34 | 0 | CDK5 | Q00535 | wp016_SK.3.93 | SK.3.93 | down | −0.970259943 | 8.79E−06 |
| 34 | 34 | 0 | CDK5 | Q00535 | wp016_SK.3.91 | SK.3.91 | down | −1.126179938 | 4.60E−06 |
| 34 | 34 | 0 | CDK5 | Q00535 | wp016_SK.3.87 | SK.3.87 | down | −0.573183814 | 8.52E−05 |
| 34 | 34 | 0 | CDK5 | Q00535 | wp016_LT2.49 | LT2.49 | down | −0.860835325 | 1.48E−05 |
| 34 | 34 | 0 | CDK5 | Q00535 | wp016_SK.3.89 | SK.3.89 | down | −0.625436388 | 5.86E−05 |
| 34 | 34 | 0 | CDK5 | Q00535 | wp056_SB1.G.187 | SB1.G.187 | down | −0.480383299 | 0.002762885 |
| 34 | 34 | 0 | CDK5 | Q00535 | wp081_TL13.150 | TL13.150 | down | −0.378638263 | 9.35E−06 |
| 34 | 34 | 0 | CDK5 | Q00535 | wp103_SK.3.91 | SK.3.91 | down | −1.043121505 | 1.00E−05 |
| 34 | 34 | 0 | CDK5 | Q00535 | wp103_SB1.G.187 | SB1.G.187 | down | −0.59240625 | 0.00012964 |
| 34 | 34 | 0 | CDK5 | Q00535 | wp104_TMX.01.160.1_0.25 | TMX.01.160.1 | down | −1.866306807 | 1.48E−07 |
| 34 | 34 | 0 | CDK5 | Q00535 | wp104_TMX.01.160.1_1 | TMX.01.160.1 | down | −1.826793697 | 1.65E−07 |
| 34 | 34 | 0 | CDK5 | Q00535 | wp105_SK.3.91 | SK.3.91 | down | −0.931040141 | 7.01E−07 |
| 34 | 34 | 0 | CDK5 | Q00535 | wp105_SB1.G.187 | SB1.G.187 | down | −0.605277609 | 1.36E−05 |
| 34 | 34 | 0 | CDK5 | Q00535 | wp107_SK.3.91 | SK.3.91 | down | −1.014483946 | 1.62E−06 |
| 34 | 34 | 0 | CDK5 | Q00535 | wp107_SB1.G.187 | SB1.G.187 | down | −0.677793934 | 1.41E−05 |
| 34 | 34 | 0 | CDK5 | Q00535 | wp114_SK.3.91 | SK.3.91 | down | −0.467996857 | 0.004142897 |
| 34 | 34 | 0 | CDK5 | Q00535 | wp131_TL12.186 | TL12.186 | down | −1.300481446 | 1.67E−07 |
| 34 | 34 | 0 | CDK5 | Q00535 | wp133_SK.3.91_2 hr | SK.3.91 | down | −0.660777891 | 1.06E−06 |
| 34 | 34 | 0 | CDK5 | Q00535 | wp133_SK.3.91_4 hr | SK.3.91 | down | −1.242095675 | 1.33E−08 |
| 34 | 34 | 0 | CDK5 | Q00535 | wp137_TL12.186 | TL12.186 | down | −1.234105823 | 8.09E−09 |
| 34 | 34 | 0 | CDK5 | Q00535 | wp137_TL13.97 | TL13.97 | down | −1.113832709 | 1.43E−08 |
| 34 | 34 | 0 | CDK5 | Q00535 | wp146_TMX.01.160 | TMX.01.160 | down | −2.901173471 | 1.67E−06 |
| 33 | 33 | 0 | CDK5 | Q00535 | wp146_TMX.02.138 | TMX.02.138 | down | −3.289852815 | 1.06E−06 |
| 33 | 33 | 0 | CDK5 | Q00535 | wp146_TMX.02.172 | TMX.02.172 | down | −2.877881154 | 1.73E−06 |
| 33 | 33 | 0 | CDK5 | Q00535 | wp146_TMX.02.174 | TMX.02.174 | down | −3.160704836 | 1.22E−06 |
| 33 | 33 | 0 | CDK5 | Q00535 | wp160_SK.3.91_1 hr | SK.3.91 | down | −0.32572193 | 0.000802895 |
| 33 | 33 | 0 | CDK5 | Q00535 | wp160_SK.3.91_2 hr | SK.3.91 | down | −0.626452987 | 6.00E−05 |
| 33 | 33 | 0 | CDK5 | Q00535 | wp160_SK.3.91_4 hr | SK.3.91 | down | −1.197063877 | 4.40E−06 |
| 33 | 33 | 0 | CDK5 | Q00535 | wp172_SB1.G.187 | SB1.G.187 | down | −0.89384384 | 2.54E−06 |
| 33 | 33 | 0 | CDK5 | Q00535 | wp172_SK.3.91 | SK.3.91 | down | −1.304147155 | 1.08E−07 |
| 33 | 33 | 0 | CDK17 | Q00537 | wp016_SK.3.91_1 uM | SK.3.91 | down | −0.750328747 | 0.000417908 |
| 33 | 33 | 0 | CDK17 | Q00537 | wp016_TL12.186 | TL12.186 | down | −0.84003685 | 0.00025954 |
| 33 | 33 | 0 | CDK17 | Q00537 | wp016_SK.3.93 | SK.3.93 | down | −0.710264828 | 0.000526001 |
| 33 | 33 | 0 | CDK17 | Q00537 | wp016_SK.3.91 | SK.3.91 | down | −0.734078769 | 0.000458138 |
| 33 | 33 | 0 | CDK17 | Q00537 | wp016_SK.3.87 | SK.3.87 | down | −0.613914798 | 0.000963948 |
| 33 | 33 | 0 | CDK17 | Q00537 | wp016_LT2.49 | LT2.49 | down | −0.754449889 | 0.000408399 |
| 33 | 33 | 0 | CDK17 | Q00537 | wp016_SK.3.89 | SK.3.89 | down | −0.605650686 | 0.001019325 |
| 33 | 33 | 0 | CDK17 | Q00537 | wp047_DD.03.106 | DD.03.106 | down | −0.720011716 | 0.000111965 |
| 33 | 33 | 0 | CDK17 | Q00537 | wp056_DD.03.156 | DD.03.156 | down | −1.824897827 | 5.89E−06 |
| 33 | 33 | 0 | CDK17 | Q00537 | wp076_MFH.5.126.1 | MFH.5.126.1 | down | −0.589449157 | 0.002138953 |
| 33 | 33 | 0 | CDK17 | Q00537 | wp080_RSS0680 | RSS0680 | down | −0.787504002 | 4.14E−05 |
| 33 | 33 | 0 | CDK17 | Q00537 | wp103_SK.3.91 | SK.3.91 | down | −0.601535761 | 0.001302903 |
| 33 | 33 | 0 | CDK17 | Q00537 | wp104_TMX.01.160.1_0.25 | TMX.01.160.1 | down | −0.778813707 | 9.19E−05 |
| 33 | 33 | 0 | CDK17 | Q00537 | wp104_TMX.01.160.1_1 | TMX.01.160.1 | down | −0.681745614 | 0.000177218 |
| 33 | 33 | 0 | CDK17 | Q00537 | wp105_SK.3.91 | SK.3.91 | down | −0.731999224 | 0.000104793 |
| 33 | 33 | 0 | CDK17 | Q00537 | wp107_SK.3.91 | SK.3.91 | down | −0.614486474 | 4.84E−06 |
| 33 | 33 | 0 | CDK17 | Q00537 | wp117_RSS0680 | RSS0680 | down | −0.719322309 | 3.80E−06 |
| 33 | 33 | 0 | CDK17 | Q00537 | wp131_TL12.186 | TL12.186 | down | −0.616539056 | 4.01E−06 |
| 33 | 33 | 0 | CDK17 | Q00537 | wp133_SK.3.91_2 hr | SK.3.91 | down | −0.371955407 | 2.99E−05 |
| 33 | 33 | 0 | CDK17 | Q00537 | wp133_SK.3.91_4 hr | SK.3.91 | down | −0.611017166 | 1.15E−07 |
| 33 | 33 | 0 | CDK17 | Q00537 | wp146_TMX.01.160 | TMX.01.160 | down | −1.626629866 | 7.99E−05 |
| 33 | 33 | 0 | CDK17 | Q00537 | wp146_TMX.02.138 | TMX.02.138 | down | −1.361060875 | 0.000152633 |
| 33 | 33 | 0 | CDK17 | Q00537 | wp146_TMX.02.174 | TMX.02.174 | down | −1.419552877 | 0.000131028 |
| 33 | 33 | 0 | CDK17 | Q00537 | wp147_DD.03.106_1 uM | DD.03.106 | down | −0.761496112 | 1.47E−05 |
| 33 | 33 | 0 | CDK17 | Q00537 | wp147_DD.03.106_2 uM | DD.03.106 | down | −0.828822697 | 9.56E−06 |
| 33 | 33 | 0 | CDK17 | Q00537 | wp147_DD.03.106_4 uM | DD.03.106 | down | −0.623169506 | 4.05E−05 |
| 33 | 33 | 0 | CDK17 | Q00537 | wp160_SK.3.91_4 hr | SK.3.91 | down | −0.395207294 | 0.002275484 |
| 33 | 33 | 0 | CDK17 | Q00537 | wp172_SB1.G.187 | SB1.G.187 | down | −0.35620722 | 0.006107982 |
| 33 | 33 | 0 | CDK17 | Q00537 | wp172_SK.3.91 | SK.3.91 | down | −0.749154192 | 4.17E−05 |
| 33 | 33 | 0 | LCK | P06239-3 | wp016_SK.3.91_1 uM | SK.3.91 | down | −1.579097427 | 7.36E−07 |
| 33 | 33 | 0 | LCK | P06239-3 | wp016_TL12.186 | TL12.186 | down | −0.712937286 | 2.33E−05 |
| 33 | 33 | 0 | LCK | P06239-3 | wp016_SK.3.93 | SK.3.93 | down | −0.779798611 | 1.58E−05 |
| 33 | 33 | 0 | LCK | P06239-3 | wp016_SK.3.91 | SK.3.91 | down | −0.918920151 | 7.76E−06 |
| 33 | 33 | 0 | LCK | P06239-3 | wp016_SK.3.87 | SK.3.87 | down | −0.348407919 | 0.000495561 |
| 33 | 33 | 0 | LCK | P06239-3 | wp016_LT2.49 | LT2.49 | down | −0.618553897 | 4.30E−05 |
| 33 | 33 | 0 | LCK | P06239-3 | wp016_SK.3.89 | SK.3.89 | down | −0.449023127 | 0.000169656 |
| 33 | 33 | 0 | LCK | P06239-3 | wp045_DB0646 | DB0646 | down | −0.563140912 | 6.01E−05 |
| 33 | 33 | 0 | LCK | P06239-3 | wp045_DB0663 | DB0663 | down | −0.582332072 | 5.20E−05 |
| 33 | 33 | 0 | LCK | P06239-3 | wp045_DB0614 | DB0614 | down | −0.330041787 | 0.000591755 |
| 33 | 33 | 0 | LCK | P06239-3 | wp047_DD.03.119 | DD.03.119 | down | −1.786910573 | 1.73E−07 |
| 33 | 33 | 0 | LCK | P06239-3 | wp056_SB1.G.187 | SB1.G.187 | down | −1.00332871 | 2.33E−05 |
| 33 | 33 | 0 | LCK | P06239-3 | wp071_SB1.G.192.1 | SB1.G.192.1 | down | −1.767095125 | 1.59E−06 |
| 33 | 33 | 0 | LCK | P06239-3 | wp071_SB1.G.181 | SB1.G.181 | down | −1.774249884 | 1.56E−06 |
| 33 | 33 | 0 | LCK | P06239-3 | wp071_SB1.G.200 | SB1.G.200 | down | −1.274825991 | 7.84E−06 |
| 33 | 33 | 0 | LCK | P06239-3 | wp074_dRAF.3 | dRAF.3 | down | −0.51294457 | 0.002382444 |
| 33 | 33 | 0 | LCK | P06239-3 | wp103_DB0646 | DB0646 | down | −0.449607013 | 0.000107021 |
| 33 | 33 | 0 | LCK | P06239-3 | wp103_SK.3.91 | SK.3.91 | down | −1.148074573 | 1.50E−06 |
| 33 | 33 | 0 | LCK | P06239-3 | wp103_SB1.G.187 | SB1.G.187 | down | −1.237475925 | 1.06E−06 |
| 33 | 33 | 0 | LCK | P06239-3 | wp107_SK.3.91 | SK.3.91 | down | −1.57497865 | 3.89E−07 |
| 33 | 33 | 0 | LCK | P06239-3 | wp107_SB1.G.187 | SB1.G.187 | down | −1.237014764 | 9.33E−08 |
| 33 | 33 | 0 | LCK | P06239-3 | wp107_DB0646 | DB0646 | down | −0.396300885 | 0.000469262 |
| 33 | 33 | 0 | LCK | P06239-3 | wp117_DB0646 | DB0646 | down | −0.420225459 | 5.63E−06 |
| 33 | 33 | 0 | LCK | P06239-3 | wp131_TL12.186 | TL12.186 | down | −1.454954971 | 3.40E−09 |
| 33 | 33 | 0 | LCK | P06239-3 | wp131_SB1.G.192.1 | SB1.G.192.1 | down | −1.700824492 | 7.30E−10 |
| 33 | 33 | 0 | LCK | P06239-3 | wp133_SK.3.91_2 hr | SK.3.91 | down | −0.535167424 | 5.88E−07 |
| 33 | 33 | 0 | LCK | P06239-3 | wp133_SK.3.91_4 hr | SK.3.91 | down | −1.276858973 | 3.35E−09 |
| 33 | 33 | 0 | LCK | P06239-3 | wp137_TL12.186 | TL12.186 | down | −1.372599858 | 2.06E−08 |
| 33 | 33 | 0 | LCK | P06239-3 | wp137_TL13.97 | TL13.97 | down | −0.872100432 | 2.58E−07 |
| 32 | 32 | 0 | ITK | Q08881 | wp016_SK.3.91_1 uM | SK.3.91 | down | −2.033076741 | 1.50E−05 |
| 32 | 32 | 0 | ITK | Q08881 | wp016_TL12.186 | TL12.186 | down | −2.342418165 | 8.10E−06 |
| 32 | 32 | 0 | ITK | Q08881 | wp016_SK.3.93 | SK.3.93 | down | −2.136467945 | 1.21E−05 |
| 32 | 32 | 0 | ITK | Q08881 | wp016_SK.3.91 | SK.3.91 | down | −2.307983121 | 8.64E−06 |
| 32 | 32 | 0 | ITK | Q08881 | wp016_SK.3.87 | SK.3.87 | down | −1.868433241 | 2.16E−05 |
| 32 | 32 | 0 | ITK | Q08881 | wp016_LT2.49 | LT2.49 | down | −2.211703589 | 1.04E−05 |
| 32 | 32 | 0 | ITK | Q08881 | wp016_SK.3.89 | SK.3.89 | down | −2.043890132 | 1.46E−05 |
| 32 | 32 | 0 | ITK | Q08881 | wp103_SK.3.91 | SK.3.91 | down | −1.66311181 | 4.07E−07 |
| 32 | 32 | 0 | ITK | Q08881 | wp107_SK.3.91 | SK.3.91 | down | −1.962610027 | 1.23E−07 |
| 32 | 32 | 0 | ITK | Q08881 | wp108_FMF.06.098.1 | FMF.06.098.1 | down | −0.355701666 | 0.001522525 |
| 32 | 32 | 0 | ITK | Q08881 | wp131_FMF.06.098.1 | FMF.06.098.1 | down | −0.362524015 | 7.26E−05 |
| 32 | 32 | 0 | ITK | Q08881 | wp131_TL12.186 | TL12.186 | down | −2.291944619 | 2.79E−07 |
| 32 | 32 | 0 | ITK | Q08881 | wp133_SK.3.91_2 hr | SK.3.91 | down | −0.832061298 | 1.18E−07 |
| 32 | 32 | 0 | ITK | Q08881 | wp133_SK.3.91_1 hr | SK.3.91 | down | −0.374079688 | 3.07E−05 |
| 32 | 32 | 0 | ITK | Q08881 | wp133_SK.3.91_4 hr | SK.3.91 | down | −1.653623168 | 2.28E−09 |
| 32 | 32 | 0 | ITK | Q08881 | wp137_TL12.186 | TL12.186 | down | −1.97295153 | 3.11E−09 |
| 32 | 32 | 0 | ITK | Q08881 | wp137_TL13.97 | TL13.97 | down | −2.28045513 | 1.38E−09 |
| 32 | 32 | 0 | ITK | Q08881 | wp160_SK.3.91_2 hr | SK.3.91 | down | −0.798994621 | 2.42E−05 |
| 32 | 32 | 0 | ITK | Q08881 | wp160_SK.3.91_4 hr | SK.3.91 | down | −1.782716599 | 9.43E−07 |
| 32 | 32 | 0 | ITK | Q08881 | wp195_BJG.04.203 | BJG.04.203 | down | −2.432580841 | 1.20E−08 |
| 32 | 32 | 0 | PTK2B | Q14289-1 | wp016_SK.3.91_1 uM | SK.3.91 | down | −3.603349471 | 3.29E−07 |
| 32 | 32 | 0 | PTK2B | Q14289-1 | wp016_TL12.186 | TL12.186 | down | −3.721976023 | 2.86E−07 |
| 32 | 32 | 0 | PTK2B | Q14289-1 | wp016_SK.3.93 | SK.3.93 | down | −3.269497752 | 5.03E−07 |
| 32 | 32 | 0 | PTK2B | Q14289-1 | wp016_SK.3.91 | SK.3.91 | down | −3.823752318 | 2.54E−07 |
| 32 | 32 | 0 | PTK2B | Q14289-1 | wp016_SK.3.87 | SK.3.87 | down | −3.217315147 | 5.40E−07 |
| 32 | 32 | 0 | PTK2B | Q14289-1 | wp016_LT2.49 | LT2.49 | down | −3.365243915 | 4.44E−07 |
| 32 | 32 | 0 | PTK2B | Q14289-1 | wp016_SK.3.89 | SK.3.89 | down | −3.554251957 | 3.50E−07 |
| 32 | 32 | 0 | PTK2B | Q14289-1 | wp045_DB0646 | DB0646 | down | −2.774260777 | 6.56E−08 |
| 32 | 32 | 0 | PTK2B | Q14289-1 | wp045_DB0663 | DB0663 | down | −1.960653 | 3.02E−07 |
| 32 | 32 | 0 | PTK2B | Q14289-1 | wp045_RSS0628 | RSS0628 | down | −1.516847855 | 9.34E−07 |
| 32 | 32 | 0 | PTK2B | Q14289-1 | wp045_DB0614 | DB0614 | down | −1.42536634 | 1.23E−06 |
| 32 | 32 | 0 | PTK2B | Q14289-1 | wp076_MFH.5.116.1 | MFH.5.116.1 | down | −2.9463713 | 5.79E−05 |
| 32 | 32 | 0 | PTK2B | Q14289-1 | wp076_MFH.5.103.1 | MFH.5.103.1 | down | −3.179570019 | 4.15E−05 |
| 32 | 32 | 0 | PTK2B | Q14289-1 | wp114_DB0646 | DB0646 | down | −1.358121951 | 3.40E−05 |
| 32 | 32 | 0 | PTK2B | Q14289-1 | wp114_SK.3.91 | SK.3.91 | down | −1.884668664 | 9.02E−06 |
| 32 | 32 | 0 | PTK2B | Q14289-1 | wp133_SK.3.91_2 hr | SK.3.91 | down | −2.026741039 | 7.71E−07 |
| 32 | 32 | 0 | PTK2B | Q14289-1 | wp133_SK.3.91_1 hr | SK.3.91 | down | −0.80557608 | 0.000147669 |
| 32 | 32 | 0 | PTK2B | Q14289-1 | wp133_SK.3.91_4 hr | SK.3.91 | down | −3.959696742 | 2.29E−07 |
| 32 | 32 | 0 | PTK2B | Q14289-1 | wp146_TMX.02.138 | TMX.02.138 | down | −1.239652247 | 0.001082279 |
| 32 | 32 | 0 | PTK2B | Q14289-1 | wp146_TMX.02.174 | TMX.02.174 | down | −1.512190093 | 0.000532736 |
| 32 | 32 | 0 | PTK2B | Q14289-1 | wp160_DB0646_1 hr | DB0646 | down | −0.395486793 | 0.000389727 |
| 32 | 32 | 0 | PTK2B | Q14289-1 | wp160_DB0646_2 hr | DB0646 | down | −1.235457844 | 4.03E−06 |
| 32 | 32 | 0 | PTK2B | Q14289-1 | wp160_DB0646_4 hr | DB0646 | down | −2.34108139 | 3.02E−07 |
| 32 | 32 | 0 | PTK2B | Q14289-1 | wp160_DB0646_8 hr | DB0646 | down | −2.599437811 | 1.97E−07 |
| 32 | 32 | 0 | PTK2B | Q14289-1 | wp160_SK.3.91_1 hr | SK.3.91 | down | −1.043491295 | 7.98E−06 |
| 32 | 32 | 0 | PTK2B | Q14289-1 | wp160_SK.3.91_2 hr | SK.3.91 | down | −2.104714531 | 4.65E−07 |
| 32 | 32 | 0 | PTK2B | Q14289-1 | wp160_SK.3.91_4 hr | SK.3.91 | down | −2.772945344 | 1.52E−07 |
| 32 | 32 | 0 | SRC | P12931-2 | wp016_SK.3.91_1 uM | SK.3.91 | down | −0.842798943 | 9.84E−05 |
| 32 | 32 | 0 | SRC | P12931-2 | wp016_TL12.186 | TL12.186 | down | −0.357298056 | 0.003463793 |
| 32 | 32 | 0 | SRC | P12931-2 | wp016_SK.3.93 | SK.3.93 | down | −0.345580911 | 0.003947676 |
| 32 | 32 | 0 | SRC | P12931-2 | wp016_SK.3.91 | SK.3.91 | down | −0.384656138 | 0.002585851 |
| 32 | 32 | 0 | SRC | P12931-2 | wp045_DB0646 | DB0646 | down | −0.415229376 | 0.000468949 |
| 32 | 32 | 0 | SRC | P12931-2 | wp045_DB0663 | DB0663 | down | −0.424678725 | 0.000426395 |
| 32 | 32 | 0 | SRC | P12931-2 | wp047_DD.03.119 | DD.03.119 | down | −0.612783818 | 4.00E−05 |
| 32 | 32 | 0 | SRC | P12931-2 | wp056_SB1.G.187 | SB1.G.187 | down | −0.604010516 | 0.000162783 |
| 32 | 32 | 0 | SRC | P12931-2 | wp071_SB1.G.192.1 | SB1.G.192.1 | down | −2.578282978 | 3.70E−07 |
| 32 | 32 | 0 | SRC | P12931-2 | wp071_SB1.G.181 | SB1.G.181 | down | −2.47795441 | 4.50E−07 |
| 32 | 32 | 0 | SRC | P12931-2 | wp071_SB1.G.200 | SB1.G.200 | down | −2.174299951 | 8.54E−07 |
| 32 | 32 | 0 | SRC | P12931-2 | wp081_TL13.150 | TL13.150 | down | −0.533083408 | 8.53E−06 |
| 32 | 32 | 0 | SRC | P12931-2 | wp081_SB1.G.192.1 | SB1.G.192.1 | down | −2.112980789 | 4.41E−09 |
| 32 | 32 | 0 | SRC | P12931-2 | wp103_SK.3.91 | SK.3.91 | down | −0.500378391 | 0.000727502 |
| 32 | 32 | 0 | SRC | P12931-2 | wp103_SB1.G.187 | SB1.G.187 | down | −0.828219013 | 7.79E−05 |
| 32 | 32 | 0 | SRC | P12931-2 | wp105_SK.3.91 | SK.3.91 | down | −0.346422296 | 0.000114933 |
| 32 | 32 | 0 | SRC | P12931-2 | wp105_SB1.G.187 | SB1.G.187 | down | −0.568306267 | 1.12E−05 |
| 32 | 32 | 0 | SRC | P12931-2 | wp105_DB0646 | DB0646 | down | −0.441237167 | 0.000139587 |
| 32 | 32 | 0 | SRC | P12931-2 | wp107_SK.3.91 | SK.3.91 | down | −0.778203152 | 1.22E−06 |
| 32 | 32 | 0 | SRC | P12931-2 | wp107_SB1.G.187 | SB1.G.187 | down | −1.205823178 | 8.13E−08 |
| 32 | 32 | 0 | SRC | P12931-2 | wp107_DB0646 | DB0646 | down | −0.427434343 | 8.93E−06 |
| 32 | 32 | 0 | SRC | P12931-2 | wp117_DB0646 | DB0646 | down | −0.383921337 | 0.001043713 |
| 32 | 32 | 0 | SRC | P12931-2 | wp131_TL12.186 | TL12.186 | down | −0.777720624 | 7.48E−06 |
| 32 | 32 | 0 | SRC | P12931-2 | wp131_SB1.G.192.1 | SB1.G.192.1 | down | −2.126597815 | 2.85E−08 |
| 32 | 32 | 0 | SRC | P12931-2 | wp133_SK.3.91_4 hr | SK.3.91 | down | −0.64075559 | 6.71E−07 |
| 32 | 32 | 0 | SRC | P12931-2 | wp137_TL12.186 | TL12.186 | down | −0.573788081 | 5.24E−05 |
| 32 | 32 | 0 | SRC | P12931-2 | wp137_TL13.97 | TL13.97 | down | −0.482431232 | 0.000132163 |
| 32 | 32 | 0 | ULK1 | O75385 | wp045_RSS0628 | RSS0628 | down | −0.558286584 | 0.007543927 |
| 32 | 32 | 0 | ULK1 | O75385 | wp045_DB0614 | DB0614 | down | −0.86904479 | 0.001304474 |
| 32 | 32 | 0 | ULK1 | O75385 | wp080_RSS0680 | RSS0680 | down | −0.405654945 | 0.000774432 |
| 32 | 32 | 0 | ULK1 | O75385 | wp103_SK.3.91 | SK.3.91 | down | −1.332428321 | 2.42E−05 |
| 32 | 32 | 0 | ULK1 | O75385 | wp105_SK.3.91 | SK.3.91 | down | −1.711815271 | 2.03E−06 |
| 32 | 32 | 0 | ULK1 | O75385 | wp107_SK.3.91 | SK.3.91 | down | −1.162173914 | 2.25E−06 |
| 32 | 32 | 0 | ULK1 | O75385 | wp108_FMF.06.098.1 | FMF.06.098.1 | down | −0.687972448 | 3.40E−05 |
| 32 | 32 | 0 | ULK1 | O75385 | wp117_RSS0680 | RSS0680 | down | −0.49956121 | 2.12E−05 |
| 32 | 32 | 0 | ULK1 | O75385 | wp117_DB0614 | DB0614 | down | −1.105238516 | 3.49E−07 |
| 32 | 32 | 0 | ULK1 | O75385 | wp131_FMF.06.098.1 | FMF.06.098.1 | down | −0.96289442 | 7.54E−07 |
| 32 | 32 | 0 | ULK1 | O75385 | wp131_TL12.186 | TL12.186 | down | −3.010390025 | 1.35E−07 |
| 32 | 32 | 0 | ULK1 | O75385 | wp133_SK.3.91_2 hr | SK.3.91 | down | −2.778568143 | 1.53E−09 |
| 32 | 32 | 0 | ULK1 | O75385 | wp133_SK.3.91_1 hr | SK.3.91 | down | −1.47214609 | 2.08E−07 |
| 32 | 32 | 0 | ULK1 | O75385 | wp133_SK.3.91_4 hr | SK.3.91 | down | −3.57223887 | 3.46E−10 |
| 32 | 32 | 0 | ULK1 | O75385 | wp134_BSJ.04.178 | BSJ.04.178 | down | −1.682936012 | 6.45E−07 |
| 32 | 32 | 0 | ULK1 | O75385 | wp137_TL12.186 | TL12.186 | down | −2.237008076 | 3.64E−08 |
| 32 | 32 | 0 | ULK1 | O75385 | wp137_TL13.97 | TL13.97 | down | −1.87718947 | 9.68E−08 |
| 32 | 32 | 0 | ULK1 | O75385 | wp152_DB1114 | DB1114 | down | −0.32490956 | 0.002348788 |
| 32 | 32 | 0 | ULK1 | O75385 | wp160_DB0646_4 hr | DB0646 | down | −0.34124989 | 0.008221883 |
| 32 | 32 | 0 | ULK1 | O75385 | wp160_DB0646_8 hr | DB0646 | down | −0.398939189 | 0.004674638 |
| 32 | 32 | 0 | ULK1 | O75385 | wp160_SK.3.91_1 hr | SK.3.91 | down | −1.465780592 | 2.87E−05 |
| 32 | 32 | 0 | ULK1 | O75385 | wp160_SK.3.91_2 hr | SK.3.91 | down | −2.582992217 | 2.90E−06 |
| 30 | 30 | 0 | ULK1 | O75385 | wp160_SK.3.91_4 hr | SK.3.91 | down | −3.084728718 | 1.41E−06 |
| 30 | 30 | 0 | ULK1 | O75385 | wp168_DB0662 | DB0662 | down | −0.358196962 | 0.000891081 |
| 30 | 30 | 0 | ULK1 | O75385 | wp168_DB1113 | DB1113 | down | −0.352005821 | 0.000521205 |
| 30 | 30 | 0 | ULK1 | O75385 | wp195_BSJ.05.026 | BSJ.05.026 | down | −0.442356322 | 0.000101453 |
| 30 | 30 | 0 | ABL2 | P42684 | wp016_SK.3.91_1 uM | SK.3.91 | down | −1.07679169 | 0.000422446 |
| 30 | 30 | 0 | ABL2 | P42684 | wp016_SK.3.93 | SK.3.93 | down | −0.527756236 | 0.007431675 |
| 30 | 30 | 0 | ABL2 | P42684 | wp016_SK.3.91 | SK.3.91 | down | −0.496303827 | 0.009343288 |
| 30 | 30 | 0 | ABL2 | P42684 | wp045_DB0646 | DB0646 | down | −0.594127502 | 0.000220278 |
| 30 | 30 | 0 | ABL2 | P42684 | wp045_DB.3.291 | DB.3.291 | down | −0.373890265 | 0.0015394 |
| 30 | 30 | 0 | ABL2 | P42684 | wp045_DB0663 | DB0663 | down | −0.60224285 | 0.000207858 |
| 30 | 30 | 0 | ABL2 | P42684 | wp056_SB1.G.187 | SB1.G.187 | down | −0.62521158 | 0.000853316 |
| 30 | 30 | 0 | ABL2 | P42684 | wp071_SB1.G.192.1 | SB1.G.192.1 | down | −2.440347814 | 2.68E−06 |
| 30 | 30 | 0 | ABL2 | P42684 | wp071_SB1.G.181 | SB1.G.181 | down | −2.404847635 | 2.88E−06 |
| 30 | 30 | 0 | ABL2 | P42684 | wp071_SB1.G.200 | SB1.G.200 | down | −2.103121652 | 5.54E−06 |
| 30 | 30 | 0 | ABL2 | P42684 | wp107_SK.3.91 | SK.3.91 | down | −1.032199977 | 6.90E−08 |
| 30 | 30 | 0 | ABL2 | P42684 | wp107_SB1.G.187 | SB1.G.187 | down | −0.645472181 | 5.72E−06 |
| 30 | 30 | 0 | ABL2 | P42684 | wp107_DB0646 | DB0646 | down | −0.534180963 | 2.63E−06 |
| 30 | 30 | 0 | ABL2 | P42684 | wp133_SK.3.91_2 hr | SK.3.91 | down | −0.345289424 | 0.00030789 |
| 30 | 30 | 0 | ABL2 | P42684 | wp133_SK.3.91_4 hr | SK.3.91 | down | −0.851901558 | 1.47E−06 |
| 30 | 30 | 0 | ABL2 | P42684 | wp137_TL12.186 | TL12.186 | down | −0.80296021 | 2.27E−06 |
| 30 | 30 | 0 | ABL2 | P42684 | wp137_TL13.97 | TL13.97 | down | −0.511550104 | 2.68E−05 |
| 30 | 30 | 0 | ABL2 | P42684 | wp152_DB0646 | DB0646 | down | −0.536206414 | 9.84E−06 |
| 30 | 30 | 0 | ABL2 | P42684 | wp152_DB1114 | DB1114 | down | −0.962203873 | 4.01E−07 |
| 30 | 30 | 0 | ABL2 | P42684 | wp160_DB0646_4 hr | DB0646 | down | −0.502861067 | 0.000360966 |
| 30 | 30 | 0 | ABL2 | P42684 | wp160_DB0646_8 hr | DB0646 | down | −0.728807383 | 8.24E−05 |
| 30 | 30 | 0 | ABL2 | P42684 | wp160_SK.3.91_4 hr | SK.3.91 | down | −0.765847142 | 6.75E−05 |
| 30 | 30 | 0 | ABL2 | P42684 | wp168_DB0662 | DB0662 | down | −1.206961737 | 8.61E−07 |
| 30 | 30 | 0 | ABL2 | P42684 | wp168_DB1113 | DB1113 | down | −1.723008009 | 6.02E−08 |
| 30 | 30 | 0 | ABL2 | P42684 | wp195_JWG.148 | JWG.148 | down | −0.516007826 | 3.26E−05 |
| 30 | 30 | 0 | CDK9 | P50750-2 | wp016_SK.3.91_1 uM | SK.3.91 | down | −1.35609318 | 2.50E−05 |
| 29 | 29 | 0 | CDK9 | P50750-2 | wp016_TL12.186 | TL12.186 | down | −0.641411736 | 0.000607441 |
| 29 | 29 | 0 | CDK9 | P50750-2 | wp016_SK.3.93 | SK.3.93 | down | −0.637606896 | 0.000622698 |
| 29 | 29 | 0 | CDK9 | P50750-2 | wp016_SK.3.91 | SK.3.91 | down | −0.651459325 | 0.000569288 |
| 29 | 29 | 0 | CDK9 | P50750-2 | wp016_SK.3.87 | SK.3.87 | down | −0.471439316 | 0.002147279 |
| 29 | 29 | 0 | CDK9 | P50750-2 | wp016_LT2.49 | LT2.49 | down | −0.646909893 | 0.00058619 |
| 29 | 29 | 0 | CDK9 | P50750-2 | wp016_SK.3.89 | SK.3.89 | down | −0.477732268 | 0.002035738 |
| 29 | 29 | 0 | CDK9 | P50750-2 | wp068_FMF.04.147.1 | FMF.04.147.1 | down | −0.914192721 | 6.42E−06 |
| 29 | 29 | 0 | CDK9 | P50750-2 | wp076_MFH.5.126.1 | MFH.5.126.1 | down | −0.700756173 | 0.000172647 |
| 29 | 29 | 0 | CDK9 | P50750-2 | wp103_SK.3.91 | SK.3.91 | down | −0.785662988 | 7.50E−05 |
| 29 | 29 | 0 | CDK9 | P50750-2 | wp105_SK.3.91 | SK.3.91 | down | −0.677520128 | 0.0003276 |
| 29 | 29 | 0 | CDK9 | P50750-2 | wp107_SK.3.91 | SK.3.91 | down | −1.200186008 | 4.37E−07 |
| 29 | 29 | 0 | CDK9 | P50750-2 | wp108_FMF.06.098.1 | FMF.06.098.1 | down | −0.723263669 | 0.000530167 |
| 29 | 29 | 0 | CDK9 | P50750-2 | wp131_FMF.06.098.1 | FMF.06.098.1 | down | −0.679067079 | 1.58E−06 |
| 29 | 29 | 0 | CDK9 | P50750-2 | wp131_TL12.186 | TL12.186 | down | −1.451335859 | 1.73E−07 |
| 29 | 29 | 0 | CDK9 | P50750-2 | wp133_SK.3.91_2 hr | SK.3.91 | down | −0.397053308 | 2.27E−05 |
| 29 | 29 | 0 | CDK9 | P50750-2 | wp133_SK.3.91_4 hr | SK.3.91 | down | −1.036526043 | 9.74E−08 |
| 29 | 29 | 0 | CDK9 | P50750-2 | wp134_BSJ.04.178 | BSJ.04.178 | down | −1.430083853 | 1.23E−06 |
| 29 | 29 | 0 | CDK9 | P50750-2 | wp137_TL12.186 | TL12.186 | down | −1.510791319 | 2.52E−08 |
| 29 | 29 | 0 | CDK9 | P50750-2 | wp137_TL13.97 | TL13.97 | down | −1.36977719 | 4.35E−08 |
| 29 | 29 | 0 | CDK9 | P50750-2 | wp146_TMX.01.160 | TMX.01.160 | down | −1.181140547 | 0.000176558 |
| 29 | 29 | 0 | CDK9 | P50750-2 | wp146_TMX.02.138 | TMX.02.138 | down | −1.889278939 | 3.20E−05 |
| 29 | 29 | 0 | CDK9 | P50750-2 | wp146_TMX.02.174 | TMX.02.174 | down | −1.666052665 | 5.06E−05 |
| 29 | 29 | 0 | CDK9 | P50750-2 | wp195_BJG.04.203 | BJG.04.203 | down | −0.522515639 | 2.20E−05 |
| 29 | 29 | 0 | CDK9 | P50750-2 | wp195_ZZ.01.31B | ZZ.01.31B | down | −0.836953513 | 1.65E−06 |
| 29 | 29 | 0 | CDK7 | P50613 | wp016_TL12.186 | TL12.186 | down | −0.760330791 | 0.001663505 |
| 29 | 29 | 0 | CDK7 | P50613 | wp016_SK.3.93 | SK.3.93 | down | −0.805680872 | 0.001313876 |
| 29 | 29 | 0 | CDK7 | P50613 | wp016_SK.3.91 | SK.3.91 | down | −0.523623461 | 0.007192476 |
| 29 | 29 | 0 | CDK7 | P50613 | wp016_SK.3.87 | SK.3.87 | down | −0.532511984 | 0.006749657 |
| 29 | 29 | 0 | CDK7 | P50613 | wp016_LT2.49 | LT2.49 | down | −1.013699708 | 0.000507662 |
| 28 | 20 | 8 | CDK7 | P50613 | wp016_SK.3.89 | SK.3.89 | down | −0.795933463 | 0.001380857 |
| 28 | 20 | 8 | CDK7 | P50613 | wp076_MFH.5.103.1 | MFH.5.103.1 | down | −0.509788101 | 0.00867353 |
| 28 | 20 | 8 | CDK7 | P50613 | wp080_RSS0680 | RSS0680 | down | −1.016539088 | 1.36E−05 |
| 28 | 20 | 8 | CDK7 | P50613 | wp104_TMX.01.160.1_0.25 | TMX.01.160.1 | down | −0.494106323 | 3.25E−05 |
| 28 | 20 | 8 | CDK7 | P50613 | wp104_TMX.01.160.1_1 | TMX.01.160.1 | down | −0.52036265 | 2.51E−05 |
| 28 | 20 | 8 | CDK7 | P50613 | wp105_SK.3.91 | SK.3.91 | down | −0.630703767 | 0.000145829 |
| 28 | 20 | 8 | CDK7 | P50613 | wp107_SK.3.91 | SK.3.91 | down | −0.463171176 | 0.000157184 |
| 28 | 20 | 8 | CDK7 | P50613 | wp108_FMF.06.098.1 | FMF.06.098.1 | down | −0.901606003 | 4.46E−05 |
| 28 | 20 | 8 | CDK7 | P50613 | wp114_SK.3.91 | SK.3.91 | down | −0.525544562 | 0.001586828 |
| 28 | 20 | 8 | CDK7 | P50613 | wp114_FMF.06.098.1 | FMF.06.098.1 | down | −1.136910231 | 7.49E−05 |
| 28 | 20 | 8 | CDK7 | P50613 | wp117_RSS0680 | RSS0680 | down | −1.219423946 | 1.09E−06 |
| 28 | 20 | 8 | CDK7 | P50613 | wp131_FMF.06.098.1 | FMF.06.098.1 | down | −0.975639434 | 4.06E−06 |
| 28 | 20 | 8 | CDK7 | P50613 | wp131_TL12.186 | TL12.186 | down | −0.644656957 | 5.04E−06 |
| 28 | 20 | 8 | CDK7 | P50613 | wp137_TL12.186 | TL12.186 | down | −0.743755805 | 4.69E−06 |
| 28 | 20 | 8 | CDK7 | P50613 | wp137_TL13.97 | TL13.97 | down | −1.049745143 | 6.99E−07 |
| 28 | 20 | 8 | CDK7 | P50613 | wp146_TMX.01.160 | TMX.01.160 | down | −0.601017224 | 0.002599465 |
| 28 | 20 | 8 | CDK7 | P50613 | wp146_TMX.02.138 | TMX.02.138 | down | −0.523894876 | 0.004172205 |
| 28 | 20 | 8 | CDK7 | P50613 | wp146_TMX.02.174 | TMX.02.174 | down | −0.583951006 | 0.002872494 |
| 28 | 20 | 8 | CDK7 | P50613 | wp172_SK.3.91 | SK.3.91 | down | −1.186215014 | 1.42E−06 |
| 28 | 20 | 8 | MAPK14 | Q16539 | wp045_DB0646 | DB0646 | down | −0.779307716 | 0.000116041 |
| 26 | 26 | 0 | MAPK14 | Q16539 | wp045_DB0663 | DB0663 | down | −0.62929522 | 0.000290089 |
| 26 | 26 | 0 | MAPK14 | Q16539 | wp047_DD.02.198 | DD.02.198 | down | −1.387296177 | 8.76E−06 |
| 26 | 26 | 0 | MAPK14 | Q16539 | wp071_SB1.G.192.1 | SB1.G.192.1 | down | −0.891493985 | 4.75E−05 |
| 26 | 26 | 0 | MAPK14 | Q16539 | wp081_TL13.178 | TL13.178 | down | −0.565852904 | 0.000138261 |
| 26 | 26 | 0 | MAPK14 | Q16539 | wp081_TL13.150 | TL13.150 | down | −0.373826968 | 0.001144606 |
| 26 | 26 | 0 | MAPK14 | Q16539 | wp081_SB1.G.192.1 | SB1.G.192.1 | down | −0.729270811 | 3.60E−05 |
| 26 | 26 | 0 | MAPK14 | Q16539 | wp103_DB0646 | DB0646 | down | −0.601308815 | 0.000569758 |
| 26 | 26 | 0 | MAPK14 | Q16539 | wp103_SB1.G.187 | SB1.G.187 | down | −0.830017101 | 0.000136774 |
| 26 | 26 | 0 | MAPK14 | Q16539 | wp107_SB1.G.187 | SB1.G.187 | down | −0.923950511 | 7.26E−06 |
| 26 | 26 | 0 | MAPK14 | Q16539 | wp107_DB0646 | DB0646 | down | −0.760089681 | 5.42E−05 |
| 26 | 26 | 0 | MAPK14 | Q16539 | wp114_DB0646 | DB0646 | down | −0.58138084 | 0.008673458 |
| 26 | 26 | 0 | MAPK14 | Q16539 | wp117_DB0646 | DB0646 | down | −0.639199412 | 0.000179431 |
| 26 | 26 | 0 | MAPK14 | Q16539 | wp131_SB1.G.192.1 | SB1.G.192.1 | down | −0.746310649 | 1.19E−06 |
| 26 | 26 | 0 | MAPK14 | Q16539 | wp152_DB0646 | DB0646 | down | −0.782079821 | 6.16E−08 |
| 26 | 26 | 0 | MAPK14 | Q16539 | wp152_DB1114 | DB1114 | down | −1.398869222 | 2.47E−09 |
| 26 | 26 | 0 | MAPK14 | Q16539 | wp160_DB0646_2 hr | DB0646 | down | −0.44041115 | 0.000172853 |
| 26 | 26 | 0 | MAPK14 | Q16539 | wp160_DB0646_4 hr | DB0646 | down | −0.716195435 | 2.46E−05 |
| 26 | 26 | 0 | MAPK14 | Q16539 | wp160_DB0646_8 hr | DB0646 | down | −1.153216291 | 3.59E−06 |
| 26 | 26 | 0 | MAPK14 | Q16539 | wp168_DB0662 | DB0662 | down | −1.001736733 | 1.19E−05 |
| 26 | 26 | 0 | MAPK14 | Q16539 | wp168_DB1113 | DB1113 | down | −1.730123115 | 6.08E−08 |
| 26 | 26 | 0 | MAPK14 | Q16539 | wp172_DB0646 | DB0646 | down | −0.646895439 | 3.21E−05 |
| 26 | 26 | 0 | MAPK14 | Q16539 | wp172_SB1.G.187 | SB1.G.187 | down | −0.519761982 | 0.00021888 |
| 26 | 26 | 0 | CLK1 | P49759-3 | wp068_FMF.04.147.1 | FMF.04.147.1 | down | −0.64616419 | 8.28E−05 |
| 26 | 26 | 0 | CLK1 | P49759-3 | wp076_MFH.5.126.1 | MFH.5.126.1 | down | −1.49375117 | 0.008904576 |
| 26 | 26 | 0 | CLK1 | P49759-3 | wp146_TMX.02.138 | TMX.02.138 | down | −0.464770994 | 0.009554042 |
| 26 | 26 | 0 | CLK1 | P49759-3 | wp146_TMX.02.174 | TMX.02.174 | down | −0.460586377 | 0.009840951 |
| 26 | 26 | 0 | MAPK9 | P45984-1 | wp045_DB0646 | DB0646 | down | −1.343811443 | 1.55E−06 |
| 26 | 26 | 0 | MAPK9 | P45984-1 | wp045_DB0663 | DB0663 | down | −0.598008775 | 5.34E−05 |
| 26 | 26 | 0 | MAPK9 | P45984-1 | wp045_DB0614 | DB0614 | down | −0.524445137 | 9.43E−05 |
| 26 | 26 | 0 | MAPK9 | P45984-1 | wp103_DB0646 | DB0646 | down | −1.045274375 | 6.88E−06 |
| 26 | 26 | 0 | MAPK9 | P45984-1 | wp104_TMX.01.160.1_0.25 | TMX.01.160.1 | down | −1.6350769 | 5.99E−07 |
| 26 | 26 | 0 | MAPK9 | P45984-1 | wp104_TMX.01.160.1_1 | TMX.01.160.1 | down | −1.496414775 | 9.43E−07 |
| 26 | 26 | 0 | MAPK9 | P45984-1 | wp105_DB0646 | DB0646 | down | −0.929045643 | 4.34E−07 |
| 26 | 26 | 0 | MAPK9 | P45984-1 | wp107_DB0646 | DB0646 | down | −1.414168678 | 3.84E−07 |
| 26 | 26 | 0 | MAPK9 | P45984-1 | wp114_DB0646 | DB0646 | down | −0.507773796 | 0.000843334 |
| 26 | 26 | 0 | MAPK9 | P45984-1 | wp117_DB0614 | DB0614 | down | −0.563964009 | 3.25E−06 |
| 26 | 26 | 0 | MAPK9 | P45984-1 | wp117_DB0646 | DB0646 | down | −1.487677537 | 3.49E−08 |
| 26 | 26 | 0 | MAPK9 | P45984-1 | wp146_TMX.01.160 | TMX.01.160 | down | −3.319928602 | 1.41E−06 |
| 26 | 26 | 0 | MAPK9 | P45984-1 | wp146_TMX.02.138 | TMX.02.138 | down | −1.518405008 | 2.48E−05 |
| 26 | 26 | 0 | MAPK9 | P45984-1 | wp146_TMX.02.172 | TMX.02.172 | down | −1.091367771 | 8.25E−05 |
| 26 | 26 | 0 | MAPK9 | P45984-1 | wp146_TMX.02.174 | TMX.02.174 | down | −1.031060044 | 0.000101439 |
| 26 | 26 | 0 | MAPK9 | P45984-1 | wp160_DB0646_2 hr | DB0646 | down | −0.755531468 | 0.000598191 |
| 26 | 26 | 0 | MAPK9 | P45984-1 | wp160_DB0646_4 hr | DB0646 | down | −1.498075655 | 3.92E−05 |
| 26 | 26 | 0 | MAPK9 | P45984-1 | wp160_DB0646_8 hr | DB0646 | down | −2.137242474 | 9.35E−06 |
| 26 | 26 | 0 | MAPK9 | P45984-1 | wp168_DB0661 | DB0661 | down | −1.961491192 | 1.90E−05 |
| 26 | 26 | 0 | MAPK9 | P45984-1 | wp168_DB0662 | DB0662 | down | −2.610967712 | 3.80E−06 |
| 26 | 26 | 0 | MAPK9 | P45984-1 | wp168_DB1113 | DB1113 | down | −1.215917261 | 1.54E−05 |
| 26 | 26 | 0 | MAPK9 | P45984-1 | wp172_DB0646 | DB0646 | down | −0.83426727 | 1.34E−05 |
| 26 | 26 | 0 | PLK1 | P53350 | wp076_MFH.5.126.1 | MFH.5.126.1 | down | −1.070945822 | 1.37E−05 |
| 26 | 26 | 0 | PLK1 | P53350 | wp076_MFH.5.115.2 | MFH.5.115.2 | down | −0.560026655 | 0.000230412 |
| 26 | 26 | 0 | PLK1 | P53350 | wp133_SK.3.91_2 hr | SK.3.91 | down | −0.580424371 | 3.54E−06 |
| 26 | 26 | 0 | PLK1 | P53350 | wp133_SK.3.91_1 hr | SK.3.91 | down | −0.559145841 | 4.97E−06 |
| 26 | 26 | 0 | PLK1 | P53350 | wp133_SK.3.91_4 hr | SK.3.91 | down | −0.748188335 | 4.64E−07 |
| 26 | 26 | 0 | PLK | P53350 | wp147_DD.03.106_4 uM | DD.03.106 | down | −0.35331461 | 0.000326515 |
| 26 | 26 | 0 | PLK1 | P53350 | wp152_DB0646 | DB0646 | down | −0.331033966 | 1.50E−05 |
| 26 | 26 | 0 | PLK1 | P53350 | wp168_DB0661 | DB0661 | down | −0.34532948 | 0.000462877 |
| 26 | 26 | 0 | STK17A | Q9UEE5 | wp016_SK.3.91_1 uM | SK.3.91 | down | −0.703243159 | 0.002477037 |
| 26 | 26 | 0 | STK17A | Q9UEE5 | wp045_RSS0628 | RSS0628 | down | −1.479770079 | 0.000153495 |
| 26 | 26 | 0 | STK17A | Q9UEE5 | wp045_DB0614 | DB0614 | down | −1.128120836 | 0.000487773 |
| 26 | 26 | 0 | STK17A | Q9UEE5 | wp076_MFH.5.126.1 | MFH.5.126.1 | down | −0.898378989 | 0.000716464 |
| 25 | 6 | 19 | STK17A | Q9UEE5 | wp076_MFH.5.115.2 | MFH.5.115.2 | down | −0.627714 | 0.003128979 |
| 25 | 6 | 19 | STK17A | Q9UEE5 | wp080_RSS0680 | RSS0680 | down | −1.118309632 | 2.69E−05 |
| 25 | 6 | 19 | STK17A | Q9UEE5 | wp088_SB1.G.194 | SB1.G.194 | down | −0.643372316 | 0.005184579 |
| 25 | 6 | 19 | STK17A | Q9UEE5 | wp103_SK.3.91 | SK.3.91 | down | −0.620086141 | 0.000712097 |
| 25 | 6 | 19 | STK17A | Q9UEE5 | wp107_SK.3.91 | SK.3.91 | down | −0.698421054 | 2.92E−05 |
| 25 | 6 | 19 | STK17A | Q9UEE5 | wp117_RSS0680 | RSS0680 | down | −1.378506034 | 6.54E−07 |
| 24 | 24 | 0 | STK17A | Q9UEE5 | wp117_DB0614 | DB0614 | down | −0.969039573 | 6.04E−06 |
| 24 | 24 | 0 | STK17A | Q9UEE5 | wp118_INY.03.041.01 | INY.03.041.01 | down | −0.436119648 | 0.00056789 |
| 24 | 24 | 0 | STK17A | Q9UEE5 | wp133_SK.3.91_4 hr | SK.3.91 | down | −0.54579227 | 5.77E−06 |
| 24 | 24 | 0 | STK17A | Q9UEE5 | wp134_DB118430 | DB118430 | down | −0.349746685 | 0.005848544 |
| 24 | 24 | 0 | STK17A | Q9UEE5 | wp134_BSJ.04.178 | BSJ.04.178 | down | −0.710684753 | 0.000159353 |
| 24 | 24 | 0 | STK17A | Q9UEE5 | wp135_INY.03.041_01_8 hr | INY.03.041_01 | down | −0.497031923 | 5.90E−06 |
| 24 | 24 | 0 | STK17A | Q9UEE5 | wp137_TL12.186 | TL12.186 | down | −0.505432864 | 0.000226348 |
| 24 | 24 | 0 | STK17A | Q9UEE5 | wp149_ZNL.03.127 | ZNL.03.127 | down | −0.460452455 | 0.001319314 |
| 24 | 24 | 0 | STK17A | Q9UEE5 | wp172_SK.3.91 | SK.3.91 | down | −0.396324151 | 0.000912131 |
| 24 | 24 | 0 | STK17A | Q9UEE5 | wp195_BSJ.05.026 | BSJ.05.026 | down | −0.52085379 | 7.52E−05 |
| 24 | 24 | 0 | STK17A | Q9UEE5 | wp195_INY.04.125.01 | INY.04.125.01 | down | −0.408381963 | 0.000272514 |
| 24 | 24 | 0 | STK17A | Q9UEE5 | wp195_JWG.123 | JWG.123 | down | −0.645060688 | 2.37E−05 |
| 24 | 24 | 0 | PDK1 | Q15118 | wp056_SB1.G.187 | SB1.G.187 | down | −0.41336033 | 0.005819413 |
| 24 | 24 | 0 | PDK1 | Q15118 | wp056_ZNL.02.096 | ZNL.02.096 | down | −0.368536174 | 0.008853328 |
| 24 | 24 | 0 | PDK1 | Q15118 | wp081_INY.01.140.1 | INY.01.140.1 | down | −0.432838141 | 0.000453469 |
| 24 | 24 | 0 | PDK1 | Q15118 | wp081_SB1.G.192.1 | SB1.G.192.1 | down | −0.338796796 | 0.001534993 |
| 24 | 24 | 0 | PDK1 | Q15118 | wp084_BSJ.04.132 | BSJ.04.132 | down | −0.75690792 | 5.17E−05 |
| 24 | 24 | 0 | PDK1 | Q15118 | wp084_BSJ.03.204 | BSJ.03.204 | down | −0.376694446 | 0.006649545 |
| 24 | 24 | 0 | PDK1 | Q15118 | wp084_BSJ.03.123 | BSJ.03.123 | down | −0.566578289 | 0.001314003 |
| 24 | 24 | 0 | PDK1 | Q15118 | wp137_INY.02.164 | INY.02.164 | down | −0.441910961 | 5.25E−05 |
| 24 | 24 | 0 | PDK1 | Q15118 | wp147_DD.03.106_1 uM | DD.03.106 | down | −0.33628597 | 0.004777925 |
| 24 | 24 | 0 | PDK1 | Q15118 | wp147_DD.03.106_4 uM | DD.03.106 | down | −0.659168915 | 0.000206032 |
| 24 | 24 | 0 | PDK1 | Q15118 | wp147_DD.03.107_1 uM | DD.03.107 | down | −0.472180486 | 0.001027553 |
| 24 | 24 | 0 | PDK1 | Q15118 | wp147_DD.03.107_2 uM | DD.03.107 | down | −0.453420879 | 0.001242305 |
| 24 | 24 | 0 | PDK1 | Q15118 | wp147_DD.03.107_4 uM | DD.03.107 | down | −1.108187559 | 1.53E−05 |
| 24 | 24 | 0 | BUB1 | O43683 | wp045_DB0646 | DB0646 | down | −0.337822948 | 0.000404375 |
| 24 | 24 | 0 | BUB1 | O43683 | wp076_MFH.5.126.1 | MFH.5.126.1 | down | −0.924796754 | 6.33E−06 |
| 24 | 24 | 0 | BUB1 | O43683 | wp076_MFH.5.115.2 | MFH.5.115.2 | down | −0.515920844 | 8.15E−05 |
| 24 | 24 | 0 | BUB1 | O43683 | wp117_DB0646 | DB0646 | down | −0.342307577 | 1.15E−05 |
| 24 | 24 | 0 | BUB1 | O43683 | wp131_TL12.186 | TL12.186 | down | −0.498269037 | 9.55E−07 |
| 24 | 24 | 0 | BUB1 | O43683 | wp133_SK.3.91_2 hr | SK.3.91 | down | −0.445630848 | 9.44E−08 |
| 24 | 24 | 0 | BUB1 | O43683 | wp133_SK.3.91_1 hr | SK.3.91 | down | −0.490663363 | 7.75E−08 |
| 24 | 24 | 0 | BUB1 | O43683 | wp133_SK.3.91_4 hr | SK.3.91 | down | −0.5962723 | 8.39E−08 |
| 24 | 24 | 0 | BUB1 | O43683 | wp134_DB118430 | DB118430 | down | −0.386647082 | 4.17E−06 |
| 24 | 24 | 0 | BUB1 | O43683 | wp137_TL12.186 | TL12.186 | down | −0.442198348 | 1.07E−06 |
| 24 | 24 | 0 | BUB1 | O43683 | wp152_DB0646 | DB0646 | down | −0.401398012 | 3.10E−06 |
| 24 | 24 | 0 | BUB1 | O43683 | wp152_DB118430 | DB118430 | down | −0.381439116 | 4.10E−06 |
| 24 | 24 | 0 | BUB1 | O43683 | wp168_DB0662 | DB0662 | down | −0.338222001 | 0.004110528 |
| 24 | 24 | 0 | STK17B | O94768 | wp016_SK.3.91_1 uM | SK.3.91 | down | −0.326951164 | 0.004535159 |
| 24 | 24 | 0 | STK17B | O94768 | wp017_WH.9533.153 | WH.9533.153 | down | −0.759461611 | 9.61E−05 |
| 24 | 24 | 0 | STK17B | O94768 | wp045_RSS0628 | RSS0628 | down | −1.074803819 | 0.000195157 |
| 24 | 24 | 0 | STK17B | O94768 | wp045_DB0614 | DB0614 | down | −0.982862621 | 0.000285947 |
| 24 | 24 | 0 | STK17B | O94768 | wp056_WH.10417.099 | WH.10417.099 | down | −1.507216946 | 5.81E−06 |
| 24 | 24 | 0 | STK17B | O94768 | wp076_MFH.5.126.1 | MFH.5.126.1 | down | −0.582074923 | 0.00070772 |
| 24 | 24 | 0 | STK17B | O94768 | wp080_RSS0680 | RSS0680 | down | −1.374601027 | 2.33E−06 |
| 24 | 24 | 0 | STK17B | O94768 | wp103_WH.10417.099 | WH.10417.099 | down | −1.292274856 | 0.000124862 |
| 24 | 24 | 0 | STK17B | O94768 | wp105_WH.10417.099 | WH.10417.099 | down | −0.792376293 | 0.000301393 |
| 24 | 24 | 0 | STK17B | O94768 | wp107_WH.10417.099 | WH.10417.099 | down | −1.472173291 | 8.44E−07 |
| 22 | 21 | 1 | STK17B | O94768 | wp117_RSS0680 | RSS0680 | down | −1.373266181 | 3.58E−07 |
| 22 | 21 | 1 | STK17B | O94768 | wp117_DB0614 | DB0614 | down | −0.77706518 | 1.34E−05 |
| 22 | 21 | 1 | STK17B | O94768 | wp133_SK.3.91_4 hr | SK.3.91 | down | −0.545010786 | 8.65E−06 |
| 22 | 21 | 1 | STK17B | O94768 | wp134_BSJ.04.178 | BSJ.04.178 | down | −1.604400387 | 9.69E−07 |
| 22 | 21 | 1 | STK17B | O94768 | wp172_WH.10417.099 | WH.10417.099 | down | −1.234267374 | 3.84E−05 |
| 22 | 21 | 1 | STK17B | O94768 | wp195_BJG.04.203 | BJG.04.203 | down | −0.5951327 | 4.00E−05 |
| 22 | 21 | 1 | STK17B | O94768 | wp195_BSJ.05.026 | BSJ.05.026 | down | −0.929925248 | 3.48E−06 |
| 22 | 21 | 1 | STK17B | O94768 | wp195_JWG.123 | JWG.123 | down | −1.871650638 | 7.13E−08 |
| 22 | 21 | 1 | MAP4K2 | Q12851 | wp045_DB0646 | DB0646 | down | −0.756875392 | 6.34E−05 |
| 22 | 21 | 1 | MAP4K2 | Q12851 | wp045_DB0663 | DB0663 | down | −0.382326125 | 0.001156628 |
| 22 | 21 | 1 | MAP4K2 | Q12851 | wp056_SB1.G.187 | SB1.G.187 | down | −1.847049166 | 3.51E−06 |
| 22 | 21 | 1 | MAP4K2 | Q12851 | wp081_TL13.150 | TL13.150 | down | −1.088316928 | 8.77E−07 |
| 22 | 21 | 1 | MAP4K2 | Q12851 | wp103_DB0646 | DB0646 | down | −0.462459335 | 0.001953735 |
| 22 | 21 | 1 | MAP4K2 | Q12851 | wp103_SB1.G.187 | SB1.G.187 | down | −1.789990451 | 4.68E−06 |
| 22 | 21 | 1 | MAP4K2 | Q12851 | wp105_SB1.G.187 | SB1.G.187 | down | −1.75749646 | 9.13E−06 |
| 22 | 21 | 1 | MAP4K2 | Q12851 | wp107_SB1.G.187 | SB1.G.187 | down | −2.047692739 | 1.74E−07 |
| 22 | 21 | 1 | MAP4K2 | Q12851 | wp107_DB0646 | DB0646 | down | −0.624462745 | 4.72E−05 |
| 22 | 21 | 1 | MAP4K2 | Q12851 | wp108_FMF.06.098.1 | FMF.06.098.1 | down | −0.550664586 | 0.000304371 |
| 22 | 21 | 1 | MAP4K2 | Q12851 | wp114_SB1.G.187 | SB1.G.187 | down | −0.751561388 | 0.006521336 |
| 22 | 21 | 1 | MAP4K2 | Q12851 | wp117_DB0646 | DB0646 | down | −0.561643029 | 9.58E−06 |
| 22 | 21 | 1 | MAP4K2 | Q12851 | wp131_FMF.06.098.1 | FMF.06.098.1 | down | −0.609234781 | 4.08E−06 |
| 22 | 13 | 9 | MAP4K2 | Q12851 | wp152_DB0646 | DB0646 | down | −0.798111946 | 3.40E−05 |
| 22 | 13 | 9 | MAP4K2 | Q12851 | wp152_DB1114 | DB1114 | down | −2.313054736 | 1.01E−07 |
| 22 | 13 | 9 | MAP4K2 | Q12851 | wp160_DB0646_4 hr | DB0646 | down | −0.613154197 | 3.98E−05 |
| 22 | 13 | 9 | MAP4K2 | Q12851 | wp160_DB0646_8 hr | DB0646 | down | −1.154960747 | 3.09E−06 |
| 22 | 13 | 9 | MAP4K2 | Q12851 | wp168_DB1113 | DB1113 | down | −2.431673116 | 2.28E−09 |
| 22 | 13 | 9 | MAP4K2 | Q12851 | wp195_BJG.04.203 | BJG.04.203 | down | −0.334455992 | 0.00012406 |
| 22 | 13 | 9 | AURKB | Q96GD4-5 | wp080_dAURK.4 | dAURK.4 | down | −0.393178964 | 0.001269893 |
| 22 | 13 | 9 | AURKB | Q96GD4-5 | wp088_SB1.G.194 | SB1.G.194 | down | −0.384607902 | 0.000437951 |
| 22 | 13 | 9 | AURKB | Q96GD4-5 | wp103_SK.3.91 | SK.3.91 | down | −0.396604772 | 0.001994977 |
| 22 | 13 | 9 | AURKB | Q96GD4-5 | wp105_SK.3.91 | SK.3.91 | down | −0.700397228 | 1.17E−05 |
| 22 | 13 | 9 | AURKB | Q96GD4-5 | wp107_SK.3.91 | SK.3.91 | down | −0.44298608 | 0.000432773 |
| 22 | 13 | 9 | AURKB | Q96GD4-5 | wp108_FMF.06.098.1 | FMF.06.098.1 | down | −0.524575185 | 0.000792221 |
| 22 | 13 | 9 | AURKB | Q96GD4-5 | wp131_FMF.06.098.1 | FMF.06.098.1 | down | −0.672588374 | 1.62E−06 |
| 22 | 8 | 14 | AURKB | Q96GD4-5 | wp131_TL12.186 | TL12.186 | down | −0.993779608 | 4.19E−07 |
| 22 | 8 | 14 | AURKB | Q96GD4-5 | wp133_SK.3.91_2 hr | SK.3.91 | down | −0.533268726 | 1.90E−05 |
| 22 | 8 | 14 | AURKB | Q96GD4-5 | wp133_SK.3.91_1 hr | SK.3.91 | down | −0.492307598 | 2.39E−05 |
| 22 | 8 | 14 | AURKB | Q96GD4-5 | wp133_SK.3.91_4 hr | SK.3.91 | down | −0.800717217 | 1.16E−06 |
| 22 | 8 | 14 | AURKB | Q96GD4-5 | wp134_BSJ.04.178 | BSJ.04.178 | down | −0.507374201 | 5.55E−05 |
| 22 | 8 | 14 | AURKB | Q96GD4-5 | wp137_TL12.186 | TL12.186 | down | −0.971135274 | 5.79E−06 |
| 22 | 8 | 14 | AURKB | Q96GD4-5 | wp137_TL13.97 | TL13.97 | down | −0.752298697 | 2.34E−05 |
| 22 | 8 | 14 | AURKB | Q96GD4-5 | wp146_TMX.02.138 | TMX.02.138 | down | −1.831984085 | 5.46E−05 |
| 22 | 22 | 0 | AURKB | Q96GD4-5 | wp146_TMX.02.172 | TMX.02.172 | down | −0.485600926 | 0.006112253 |
| 22 | 22 | 0 | AURKB | Q96GD4-5 | wp146_TMX.02.174 | TMX.02.174 | down | −1.577844375 | 9.40E−05 |
| 22 | 22 | 0 | MKNK2 | Q9HBH9-1 | wp045_DB0646 | DB0646 | down | −0.446301615 | 0.001907301 |
| 22 | 22 | 0 | MKNK2 | Q9HBH9-1 | wp045_DB0663 | DB0663 | down | −0.479799029 | 0.001419054 |
| 22 | 22 | 0 | MKNK2 | Q9HBH9-1 | wp080_RSS0680 | RSS0680 | down | −0.498658341 | 0.005129721 |
| 22 | 22 | 0 | MKNK2 | Q9HBH9-1 | wp117_RSS0680 | RSS0680 | down | −0.507454269 | 0.000294869 |
| 22 | 22 | 0 | MKNK2 | Q9HBH9-1 | wp117_DB0646 | DB0646 | down | −0.379412796 | 0.001609806 |
| 22 | 22 | 0 | MKNK2 | Q9HBH9-1 | wp118_ZNL.02.096 | ZNL.02.096 | down | −0.475085361 | 0.003387879 |
| 22 | 22 | 0 | MKNK2 | Q9HBH9-1 | wp133_SK.3.91_2 hr | SK.3.91 | down | −0.530274332 | 0.00034479 |
| 22 | 22 | 0 | MKNK2 | Q9HBH9-1 | wp133_SK.3.91_1 hr | SK.3.91 | down | −0.516844462 | 0.000396836 |
| 22 | 22 | 0 | MKNK2 | Q9HBH9-1 | wp133_SK.3.91_4 hr | SK.3.91 | down | −0.853747073 | 2.12E−05 |
| 22 | 22 | 0 | MKNK2 | Q9HBH9-1 | wp160_DB0646_4 hr | DB0646 | down | −0.522623559 | 0.004888907 |
| 22 | 22 | 0 | MKNK2 | Q9HBH9-1 | wp160_DB0646_8 hr | DB0646 | down | −0.642234498 | 0.002266539 |
| 22 | 22 | 0 | MKNK2 | Q9HBH9-1 | wp168_DB0661 | DB0661 | down | −0.509353306 | 0.001418944 |
| 22 | 22 | 0 | MKNK2 | Q9HBH9-1 | wp168_DB0662 | DB0662 | down | −0.41847412 | 0.00288936 |
| 22 | 22 | 0 | CDK13 | Q14004-1 | wp016_SK.3.91_1 uM | SK.3.91 | down | −0.5748761 | 0.000182847 |
| 22 | 22 | 0 | CDK13 | Q14004-1 | wp016_TL12.186 | TL12.186 | down | −0.588491228 | 0.00016551 |
| 22 | 22 | 0 | CDK13 | Q14004-1 | wp016_SK.3.93 | SK.3.93 | down | −0.5636444 | 0.000198845 |
| 22 | 22 | 0 | CDK13 | Q14004-1 | wp016_SK.3.91 | SK.3.91 | down | −0.604562469 | 0.000147559 |
| 22 | 22 | 0 | CDK13 | Q14004-1 | wp016_LT2.49 | LT2.49 | down | −0.383898191 | 0.000994833 |
| 22 | 22 | 0 | CDK13 | Q14004-1 | wp076_MFH.5.126.1 | MFH.5.126.1 | down | −0.484646741 | 0.000650367 |
| 22 | 22 | 0 | CDK13 | Q14004-1 | wp103_SK.3.91 | SK.3.91 | down | −0.483299384 | 3.57E−05 |
| 21 | 21 | 0 | CDK13 | Q14004-1 | wp105_SK.3.91 | SK.3.91 | down | −0.46486682 | 1.29E−05 |
| 21 | 21 | 0 | CDK13 | Q14004-1 | wp107_SK.3.91 | SK.3.91 | down | −0.711191906 | 4.74E−07 |
| 21 | 21 | 0 | CDK13 | Q14004-1 | wp108_FMF.06.098.1 | FMF.06.098.1 | down | −0.366410621 | 6.64E−05 |
| 21 | 21 | 0 | CDK13 | Q14004-1 | wp131_FMF.06.098.1 | FMF.06.098.1 | down | −0.498133191 | 5.48E−07 |
| 21 | 21 | 0 | CDK13 | Q14004-1 | wp131_TL12.186 | TL12.186 | down | −0.790304564 | 6.16E−07 |
| 21 | 21 | 0 | CDK13 | Q14004-1 | wp133_SK.3.91_4 hr | SK.3.91 | down | −0.556235442 | 3.44E−06 |
| 21 | 21 | 0 | CDK13 | Q14004-1 | wp137_TL12.186 | TL12.186 | down | −0.706207373 | 5.46E−07 |
| 21 | 21 | 0 | CDK13 | Q14004-1 | wp137_TL13.97 | TL13.97 | down | −0.735943464 | 4.34E−07 |
| 21 | 21 | 0 | CDK13 | Q14004-1 | wp160_SK.3.91_4 hr | SK.3.91 | down | −0.480507777 | 0.000100188 |
| 21 | 21 | 0 | CDK13 | Q14004-1 | wp172_SK.3.91 | SK.3.91 | down | −0.859003452 | 3.21E−06 |
| 21 | 21 | 0 | PDK2 | Q15119-1 | wp056_SB1.G.187 | SB1.G.187 | down | −0.401580892 | 0.006939818 |
| 21 | 21 | 0 | PDK2 | Q15119-1 | wp056_ZNL.02.096 | ZNL.02.096 | down | −0.556081506 | 0.002022456 |
| 21 | 21 | 0 | PDK2 | Q15119-1 | wp081_INY.01.140.1 | INY.01.140.1 | down | −0.609443212 | 0.006562827 |
| 21 | 21 | 0 | PDK2 | Q15119-1 | wp084_BSJ.04.132 | BSJ.04.132 | down | −0.528919274 | 0.000152711 |
| 21 | 21 | 0 | PDK2 | Q15119-1 | wp084_BSJ.03.123 | BSJ.03.123 | down | −0.361524098 | 0.003880697 |
| 21 | 21 | 0 | PDK2 | Q15119-1 | wp118_ZNL.02.096 | ZNL.02.096 | down | −0.370034183 | 0.00602864 |
| 21 | 21 | 0 | PDK2 | Q15119-1 | wp137_TL12.186 | TL12.186 | down | −0.502198063 | 0.000412792 |
| 21 | 21 | 0 | PDK2 | Q15119-1 | wp137_DD.03.107 | DD.03.107 | down | −0.338271469 | 0.002918164 |
| 21 | 21 | 0 | PDK2 | Q15119-1 | wp147_DD.03.106_4 uM | DD.03.106 | down | −0.726990522 | 0.000220557 |
| 21 | 21 | 0 | PDK2 | Q15119-1 | wp147_DD.03.107_1 uM | DD.03.107 | down | −0.496328335 | 0.001372328 |
| 21 | 21 | 0 | PDK2 | Q15119-1 | wp147_DD.03.107_2 uM | DD.03.107 | down | −0.535077075 | 0.000965484 |
| 21 | 21 | 0 | PDK2 | Q15119-1 | wp147_DD.03.107_4 uM | DD.03.107 | down | −0.92690348 | 6.61E−05 |
| 21 | 21 | 0 | TTK | P33981 | wp045_DB0646 | DB0646 | down | −0.575747412 | 4.71E−05 |
| 21 | 21 | 0 | TTK | P33981 | wp076_MFH.5.126.1 | MFH.5.126.1 | down | −0.441231164 | 0.000996416 |
| 21 | 21 | 0 | TTK | P33981 | wp076_MFH.5.115.2 | MFH.5.115.2 | down | −0.324867852 | 0.003476074 |
| 21 | 21 | 0 | TTK | P33981 | wp103_SK.3.91 | SK.3.91 | down | −0.326674585 | 0.001362336 |
| 20 | 13 | 7 | TTK | P33981 | wp107_SK.3.91 | SK.3.91 | down | −0.478068006 | 0.000449444 |
| 20 | 13 | 7 | TTK | P33981 | wp107_DB0646 | DB0646 | down | −0.37196466 | 0.000514811 |
| 20 | 13 | 7 | TTK | P33981 | wp117_DB0646 | DB0646 | down | −0.426697105 | 1.37E−05 |
| 20 | 13 | 7 | TTK | P33981 | wp133_SK.3.91_2 hr | SK.3.91 | down | −0.383344865 | 2.65E−05 |
| 20 | 13 | 7 | TTK | P33981 | wp133_SK.3.91_4 hr | SK.3.91 | down | −0.505150278 | 2.03E−06 |
| 20 | 13 | 7 | TTK | P33981 | wp146_TMX.02.138 | TMX.02.138 | down | −0.61776087 | 0.005673393 |
| 20 | 13 | 7 | TTK | P33981 | wp146_TMX.02.172 | TMX.02.172 | down | −0.754214815 | 0.002867671 |
| 20 | 13 | 7 | TTK | P33981 | wp146_TMX.02.174 | TMX.02.174 | down | −0.722511916 | 0.003326096 |
| 20 | 13 | 7 | TTK | P33981 | wp152_DB0646 | DB0646 | down | −0.513065781 | 5.99E−06 |
| 20 | 13 | 7 | TTK | P33981 | wp160_DB0646_4 hr | DB0646 | down | −0.512984136 | 0.000104191 |
| 20 | 13 | 7 | TTK | P33981 | wp160_DB0646_8 hr | DB0646 | down | −0.705669443 | 2.90E−05 |
| 20 | 13 | 7 | TTK | P33981 | wp164_SB1.G.194 | SB1.G.194 | down | −0.427947746 | 1.26E−05 |
| 20 | 13 | 7 | MARK4 | Q96L34-1 | wp080_RSS0680 | RSS0680 | down | −0.453701948 | 0.004671951 |
| 20 | 20 | 0 | MARK4 | Q96L34-1 | wp103_SK.3.91 | SK.3.91 | down | −1.326457843 | 0.001074083 |
| 20 | 20 | 0 | MARK4 | Q96L34-1 | wp107_SK.3.91 | SK.3.91 | down | −1.291559664 | 0.000212105 |
| 20 | 20 | 0 | MARK4 | Q96L34-1 | wp108_FMF.06.098.1 | FMF.06.098.1 | down | −0.904130662 | 0.000107293 |
| 20 | 20 | 0 | MARK4 | Q96L34-1 | wp114_SK.3.91 | SK.3.91 | down | −1.300077774 | 0.000446917 |
| 20 | 20 | 0 | MARK4 | Q96L34-1 | wp114_FMF.06.098.1 | FMF.06.098.1 | down | −0.722447833 | 0.004288221 |
| 20 | 20 | 0 | MARK4 | Q96L34-1 | wp131_FMF.06.098.1 | FMF.06.098.1 | down | −1.110656659 | 4.39E−05 |
| 20 | 20 | 0 | MARK4 | Q96L34-1 | wp131_TL12.186 | TL12.186 | down | −1.828541386 | 0.000113078 |
| 20 | 20 | 0 | MARK4 | Q96L34-1 | wp133_SK.3.91_2 hr | SK.3.91 | down | −0.628192252 | 0.005144436 |
| 20 | 20 | 0 | MARK4 | Q96L34-1 | wp133_SK.3.91_4 hr | SK.3.91 | down | −1.537104715 | 5.37E−05 |
| 20 | 20 | 0 | MARK4 | Q96L34-1 | wp134_BSJ.04.178 | BSJ.04.178 | down | −0.992989178 | 0.000161287 |
| 20 | 20 | 0 | MARK4 | Q96L34-1 | wp137_TL12.186 | TL12.186 | down | −1.297877926 | 3.93E−05 |
| 20 | 20 | 0 | MARK4 | Q96L34-1 | wp137_TL13.97 | TL13.97 | down | −1.739808181 | 7.96E−06 |
| 20 | 20 | 0 | MARK4 | Q96L34-1 | wp160_SK.3.91_4 hr | SK.3.91 | down | −1.528759628 | 0.000323598 |
| 20 | 20 | 0 | MARK4 | Q96L34-1 | wp195_BSJ.05.026 | BSJ.05.026 | down | −1.570806334 | 2.56E−07 |
| 20 | 20 | 0 | MARK4 | Q96L34-1 | wp195_JWG.123 | JWG.123 | down | −1.532230448 | 2.94E−07 |
| 20 | 20 | 0 | PRKAA1 | Q13131-2 | wp016_SK.3.91_1 uM | SK.3.91 | down | −0.797731139 | 2.35E−05 |
| 20 | 20 | 0 | PRKAA1 | Q13131-2 | wp016_TL12.186 | TL12.186 | down | −0.43395561 | 0.00031855 |
| 20 | 20 | 0 | PRKAA1 | Q13131-2 | wp016_SK.3.93 | SK.3.93 | down | −0.473826793 | 0.000219592 |
| 20 | 20 | 0 | PRKAA1 | Q13131-2 | wp016_SK.3.91 | SK.3.91 | down | −0.491442825 | 0.000188052 |
| 20 | 20 | 0 | PRKAA1 | Q13131-2 | wp016_LT2.49 | LT2.49 | down | −0.373869432 | 0.000595471 |
| 20 | 20 | 0 | PRKAA1 | Q13131-2 | wp103_SK.3.91 | SK.3.91 | down | −0.501243875 | 1.30E−05 |
| 20 | 20 | 0 | PRKAA1 | Q13131-2 | wp105_SK.3.91 | SK.3.91 | down | −0.441320028 | 4.16E−05 |
| 20 | 20 | 0 | PRKAA1 | Q13131-2 | wp107_SK.3.91 | SK.3.91 | down | −0.635209618 | 8.37E−07 |
| 20 | 20 | 0 | PRKAA1 | Q13131-2 | wp131_FMF.06.098.1 | FMF.06.098.1 | down | −0.369502595 | 0.000803523 |
| 20 | 20 | 0 | PRKAA1 | Q13131-2 | wp131_TL12.186 | TL12.186 | down | −0.598752042 | 1.67E−05 |
| 20 | 20 | 0 | PRKAA1 | Q13131-2 | wp133_SK.3.91_2 hr | SK.3.91 | down | −0.380428072 | 1.85E−05 |
| 20 | 20 | 0 | PRKAA1 | Q13131-2 | wp133_SK.3.91_4 hr | SK.3.91 | down | −0.675169726 | 2.11E−07 |
| 20 | 20 | 0 | PRKAA1 | Q13131-2 | wp137_TL12.186 | TL12.186 | down | −0.640995546 | 7.56E−08 |
| 20 | 20 | 0 | PRKAA1 | Q13131-2 | wp137_TL13.97 | TL13.97 | down | −0.667844023 | 6.02E−08 |
| 20 | 20 | 0 | PRKAA1 | Q13131-2 | wp146_TMX.02.138 | TMX.02.138 | down | −0.712488152 | 8.19E−05 |
| 20 | 20 | 0 | PRKAA1 | Q13131-2 | wp146_TMX.02.174 | TMX.02.174 | down | −1.621947471 | 4.06E−06 |
| 20 | 20 | 0 | RIPK1 | Q13546 | wp045_DB0646 | DB0646 | down | −0.711532023 | 8.05E−06 |
| 20 | 20 | 0 | RIPK1 | Q13546 | wp056_SB1.G.187 | SB1.G.187 | down | −1.288162799 | 2.07E−06 |
| 20 | 20 | 0 | RIPK1 | Q13546 | wp081_TL13.150 | TL13.150 | down | −0.667008571 | 1.05E−06 |
| 20 | 20 | 0 | RIPK1 | Q13546 | wp103_DB0646 | DB0646 | down | −0.479600728 | 3.98E−05 |
| 20 | 20 | 0 | RIPK1 | Q13546 | wp103_SB1.G.187 | SB1.G.187 | down | −1.766751072 | 1.02E−07 |
| 20 | 20 | 0 | RIPK1 | Q13546 | wp105_SB1.G.187 | SB1.G.187 | down | −1.14124949 | 8.06E−07 |
| 20 | 20 | 0 | RIPK1 | Q13546 | wp105_DB0646 | DB0646 | down | −0.423293016 | 2.40E−05 |
| 20 | 20 | 0 | RIPK1 | Q13546 | wp107_SB1.G.187 | SB1.G.187 | down | −1.501837279 | 2.71E−08 |
| 20 | 20 | 0 | RIPK1 | Q13546 | wp107_DB0646 | DB0646 | down | −0.512099681 | 4.97E−06 |
| 20 | 18 | 2 | RIPK1 | Q13546 | wp117_DB0646 | DB0646 | down | −0.616022806 | 1.05E−06 |
| 20 | 18 | 2 | RIPK1 | Q13546 | wp152_DB0646 | DB0646 | down | −0.549514383 | 8.14E−07 |
| 20 | 18 | 2 | RIPK1 | Q13546 | wp152_DB1114 | DB1114 | down | −2.278933703 | 3.14E−10 |
| 20 | 18 | 2 | RIPK1 | Q13546 | wp160_DB0646_4 hr | DB0646 | down | −0.390736976 | 4.17E−05 |
| 20 | 18 | 2 | RIPK1 | Q13546 | wp160_DB0646_8 hr | DB0646 | down | −0.874536698 | 1.61E−06 |
| 20 | 18 | 2 | RIPK1 | Q13546 | wp168_DB1113 | DB1113 | down | −1.989552705 | 5.92E−10 |
| 20 | 18 | 2 | RIPK1 | Q13546 | wp172_SB1.G.187 | SB1.G.187 | down | −0.965613654 | 4.34E−06 |
| 20 | 18 | 2 | COQ8A | Q8N160-1 | wp056_SB1.G.187 | SB1.G.187 | down | −0.388478968 | 0.001770385 |
| 20 | 18 | 2 | COQ8A | Q8N160-1 | wp056_ZNL.02.096 | ZNL.02.096 | down | −0.431708874 | 0.001168963 |
| 20 | 18 | 2 | COQ8A | Q8N160-1 | wp084_BSJ.04.132 | BSJ.04.132 | down | −0.593870793 | 0.000701486 |
| 20 | 18 | 2 | COQ8A | Q8N160-1 | wp084_BSJ.03.123 | BSJ.03.123 | down | −0.461110627 | 0.003115092 |
| 20 | 18 | 2 | COQ8A | Q8N160-1 | wp118_ZNL.02.096 | ZNL.02.096 | down | −0.423008551 | 0.004146656 |
| 20 | 18 | 2 | COQ8A | Q8N160-1 | wp137_TL12.186 | TL12.186 | down | −0.45637325 | 7.52E−05 |
| 20 | 18 | 2 | COQBA | Q8N160-1 | wp147_DD.03.106_1 uM | DD.03.106 | down | −0.354760985 | 0.000936386 |
| 20 | 18 | 2 | COQ8A | Q8N160-1 | wp147_DD.03.106_4 uM | DD.03.106 | down | −0.815852084 | 1.53E−05 |
| 20 | 18 | 2 | COQ8A | Q8N160-1 | wp147_DD.03.107_1 uM | DD.03.107 | down | −0.667791067 | 4.22E−05 |
| 20 | 18 | 2 | COQ8A | Q8N160-1 | wp147_DD.03.107_2 uM | DD.03.107 | down | −0.628794333 | 5.71E−05 |
| 20 | 18 | 2 | COQ8A | Q8N160-1 | wp147_DD.03.107_4 uM | DD.03.107 | down | −1.120971303 | 3.04E−06 |
| 20 | 20 | 0 | COQ8A | Q8N160-1 | wp160_SK.3.91_1 hr | SK.3.91 | down | −0.795882741 | 0.005519857 |
| 20 | 20 | 0 | MAP3K11 | Q16584 | wp103_SK.3.91 | SK.3.91 | down | −0.783773252 | 0.007056971 |
| 20 | 20 | 0 | MAP3K11 | Q16584 | wp107_SK.3.91 | SK.3.91 | down | −2.182212314 | 4.43E−06 |
| 20 | 20 | 0 | MAP3K11 | Q16584 | wp107_DB0646 | DB0646 | down | −0.50772061 | 0.003730661 |
| 20 | 20 | 0 | MAP3K11 | Q16584 | wp114_SK.3.91 | SK.3.91 | down | −0.477370767 | 0.009991372 |
| 20 | 20 | 0 | MAP3K11 | Q16584 | wp117_DB0614 | DB0614 | down | −0.729137159 | 0.000123191 |
| 20 | 20 | 0 | MAP3K11 | Q16584 | wp117_DB0646 | DB0646 | down | −0.430830472 | 0.001685503 |
| 20 | 20 | 0 | MAP3K11 | Q16584 | wp131_TL12.186 | TL12.186 | down | −1.794979343 | 2.73E−06 |
| 20 | 20 | 0 | MAP3K11 | Q16584 | wp133_SK.3.91_2 hr | SK.3.91 | down | −0.844133518 | 5.78E−05 |
| 20 | 20 | 0 | MAP3K11 | Q16584 | wp133_SK.3.91_1 hr | SK.3.91 | down | −0.398589167 | 0.00258124 |
| 20 | 20 | 0 | MAP3K11 | Q16584 | wp133_SK.3.91_4 hr | SK.3.91 | down | −1.486672303 | 3.85E−06 |
| 20 | 20 | 0 | MAP3K11 | Q16584 | wp137_TL12.186 | TL12.186 | down | −1.492825956 | 2.39E−05 |
| 20 | 20 | 0 | MAP3K11 | Q16584 | wp137_TL13.97 | TL13.97 | down | −0.762589855 | 0.000823212 |
| 20 | 20 | 0 | MAP3K11 | Q16584 | wp146_TMX.02.138 | TMX.02.138 | down | −0.924130679 | 0.004876944 |
| 20 | 20 | 0 | MAP3K11 | Q16584 | wp146_TMX.02.174 | TMX.02.174 | down | −1.87373821 | 0.00040632 |
| 20 | 20 | 0 | MAP3K11 | Q16584 | wp168_DB0662 | DB0662 | down | −0.728818784 | 0.004453503 |
| 20 | 20 | 0 | MAP3K20 | Q9NYL2-2 | wp045_DB0646 | DB0646 | down | −1.181873282 | 0.00020097 |
| 20 | 20 | 0 | MAP3K20 | Q9NYL2-2 | wp045_DB0663 | DB0663 | down | −0.801161678 | 0.001037343 |
| 20 | 20 | 0 | MAP3K20 | Q9NYL2-2 | wp074_dRAF.3 | dRAF.3 | down | −1.859583794 | 0.000133792 |
| 20 | 20 | 0 | MAP3K20 | Q9NYL2-2 | wp081_TL13.178 | TL13.178 | down | −1.407099161 | 7.33E−06 |
| 19 | 19 | 0 | MAP3K20 | Q9NYL2-2 | wp103_DB0646 | DB0646 | down | −0.700404917 | 0.001527827 |
| 19 | 19 | 0 | MAP3K20 | Q9NYL2-2 | wp103_SB1.G.187 | SB1.G.187 | down | −0.620300493 | 0.002543885 |
| 19 | 19 | 0 | MAP3K20 | Q9NYL2-2 | wp107_SB1.G.187 | SB1.G.187 | down | −0.438657621 | 0.001097564 |
| 19 | 19 | 0 | MAP3K20 | Q9NYL2-2 | wp107_DB0646 | DB0646 | down | −0.782539995 | 5.84E−05 |
| 19 | 19 | 0 | MAP3K20 | Q9NYL2-2 | wp114_DB0646 | DB0646 | down | −0.896230633 | 0.000515996 |
| 19 | 19 | 0 | MAP3K20 | Q9NYL2-2 | wp114_SB1.G.187 | SB1.G.187 | down | −0.784604423 | 0.000869797 |
| 19 | 19 | 0 | MAP3K20 | Q9NYL2-2 | wp117_DB0646 | DB0646 | down | −0.670081326 | 0.000224912 |
| 19 | 19 | 0 | MAP3K20 | Q9NYL2-2 | wp147_DD.03.106_2 uM | DD.03.106 | down | −0.738820542 | 0.000194061 |
| 19 | 19 | 0 | MAP3K20 | Q9NYL2-2 | wp147_DD.03.106_4 uM | DD.03.106 | down | −0.490116498 | 0.001389577 |
| 19 | 19 | 0 | MAP3K20 | Q9NYL2-2 | wp168_DB0662 | DB0662 | down | −1.005615673 | 0.000692956 |
| 19 | 19 | 0 | MAP3K20 | Q9NYL2-2 | wp168_DB1113 | DB1113 | down | −0.969258952 | 0.000860081 |
| 19 | 19 | 0 | CDK16 | Q00536-2 | wp080_RSS0680 | RSS0680 | down | −1.001707699 | 0.003112447 |
| 19 | 19 | 0 | CDK16 | Q00536-2 | wp104_TMX.01.160.1_0.25 | TMX.01.160.1 | down | −0.864630021 | 2.77E−06 |
| 19 | 19 | 0 | CDK16 | Q00536-2 | wp104_TMX.01.160.1_1 | TMX.01.160.1 | down | −0.600949769 | 1.75E−05 |
| 19 | 19 | 0 | CDK16 | Q00536-2 | wp105_SK.3.91 | SK.3.91 | down | −0.634552283 | 0.000692107 |
| 19 | 19 | 0 | CDK16 | Q00536-2 | wp107_SK.3.91 | SK.3.91 | down | −0.470056143 | 0.004290964 |
| 19 | 19 | 0 | CDK16 | Q00536-2 | wp117_RSS0680 | RSS0680 | down | −0.938337073 | 0.000153757 |
| 19 | 19 | 0 | CDK16 | Q00536-2 | wp117_DB0614 | DB0614 | down | −0.552810287 | 0.00330184 |
| 19 | 19 | 0 | CDK16 | Q00536-2 | wp137_DD.03.106 | DD.03.106 | down | −1.175374219 | 0.00295829 |
| 19 | 14 | 5 | CDK16 | Q00536-2 | wp146_TMX.01.160 | TMX.01.160 | down | −1.110034094 | 0.000263364 |
| 19 | 14 | 5 | CDK16 | Q00536-2 | wp146_TMX.02.138 | TMX.02.138 | down | −1.433683784 | 0.000104222 |
| 19 | 14 | 5 | CDK16 | Q00536-2 | wp146_TMX.02.174 | TMX.02.174 | down | −1.176051602 | 0.000213735 |
| 19 | 14 | 5 | CDK16 | Q00536-2 | wp147_DD.03.106_1 uM | DD.03.106 | down | −0.542670852 | 0.001443978 |
| 19 | 14 | 5 | CDK16 | Q00536-2 | wp147_DD.03.106_2 uM | DD.03.106 | down | −0.538513115 | 0.001496327 |
| 19 | 14 | 5 | CDK16 | Q00536-2 | wp147_DD.03.106_4 uM | DD.03.106 | down | −0.439269573 | 0.003756564 |
| 19 | 14 | 5 | MAP4K1 | Q92918 | wp016_SK.3.91_1 uM | SK.3.91 | down | −0.667711609 | 0.000109054 |
| 19 | 14 | 5 | MAP4K1 | Q92918 | wp045_DB0646 | DB0646 | down | −0.478867284 | 9.11E−05 |
| 19 | 14 | 5 | MAP4K1 | Q92918 | wp103_SK.3.91 | SK.3.91 | down | −0.509880823 | 4.35E−05 |
| 19 | 14 | 5 | MAP4K1 | Q92918 | wp107_SK.3.91 | SK.3.91 | down | −0.70358008 | 1.66E−06 |
| 19 | 14 | 5 | MAP4K1 | Q92918 | wp131_TL12.186 | TL12.186 | down | −0.458117863 | 3.25E−06 |
| 19 | 14 | 5 | MAP4K1 | Q92918 | wp133_SK.3.91_4 hr | SK.3.91 | down | −0.552836113 | 5.16E−07 |
| 19 | 14 | 5 | MAP4K1 | Q92918 | wp137_TL12.186 | TL12.186 | down | −0.613566557 | 1.43E−06 |
| 19 | 14 | 5 | MAP4K1 | Q92918 | wp152_DB0646 | DB0646 | down | −0.363815378 | 2.16E−05 |
| 19 | 19 | 0 | MAP4K1 | Q92918 | wp152_DB1114 | DB1114 | down | −0.661140844 | 8.32E−07 |
| 19 | 19 | 0 | MAP4K1 | Q92918 | wp160_DB0646_4 hr | DB0646 | down | −0.322808107 | 0.00020974 |
| 19 | 19 | 0 | MAP4K1 | Q92918 | wp160_DB0646_8 hr | DB0646 | down | −0.57851629 | 2.02E−05 |
| 19 | 19 | 0 | MAP4K1 | Q92918 | wp160_SK.3.91_4 hr | SK.3.91 | down | −0.481745802 | 4.23E−05 |
| 19 | 19 | 0 | MAP4K1 | Q92918 | wp168_DB0662 | DB0662 | down | −0.40672217 | 0.002996687 |
| 19 | 19 | 0 | MAP4K1 | Q92918 | wp168_DB1113 | DB1113 | down | −0.846309692 | 7.29E−05 |
| 19 | 19 | 0 | PLK4 | O00444 | wp103_SK.3.91 | SK.3.91 | down | −1.242985614 | 0.000539942 |
| 19 | 19 | 0 | PLK4 | O00444 | wp107_SK.3.91 | SK.3.91 | down | −1.035623055 | 0.003133349 |
| 19 | 19 | 0 | PLK4 | O00444 | wp117_DB0614 | DB0614 | down | −0.541443756 | 0.003734544 |
| 19 | 19 | 0 | PLK4 | O00444 | wp131_FMF.06.098.1 | FMF.06.098.1 | down | −0.743466693 | 0.000309399 |
| 19 | 19 | 0 | PLK4 | O00444 | wp131_TL12.186 | TL12.186 | down | −1.55864407 | 7.83E−05 |
| 19 | 19 | 0 | PLK4 | O00444 | wp133_SK.3.91_2 hr | SK.3.91 | down | −1.522686694 | 1.28E−05 |
| 19 | 19 | 0 | PLK4 | O00444 | wp133_SK.3.91_1 hr | SK.3.91 | down | −0.725974576 | 0.00073332 |
| 19 | 19 | 0 | PLK4 | O00444 | wp133_SK.3.91_4 hr | SK.3.91 | down | −2.258408969 | 6.80E−07 |
| 19 | 19 | 0 | PLK4 | O00444 | wp137_TL12.186 | TL12.186 | down | −1.158756871 | 0.000733279 |
| 19 | 19 | 0 | PLK4 | O00444 | wp137_TL13.97 | TL13.97 | down | −1.048528828 | 0.001208381 |
| 19 | 19 | 0 | PLK4 | O00444 | wp160_SK.3.91_2 hr | SK.3.91 | down | −0.926622204 | 0.001211434 |
| 19 | 19 | 0 | PLK4 | O00444 | wp160_SK.3.91_4 hr | SK.3.91 | down | −1.168801538 | 0.000490033 |
| 19 | 19 | 0 | PLK4 | O00444 | wp195_BJG.04.203 | BJG.04.203 | down | −0.378392207 | 0.000985426 |
| 17 | 17 | 0 | UHMK1 | Q8TAS1 | wp045_DB0646 | DB0646 | down | −0.394542211 | 0.00198787 |
| 17 | 17 | 0 | UHMK1 | Q8TAS1 | wp076_MFH.5.126.1 | MFH.5.126.1 | down | −0.478853842 | 0.008416259 |
| 17 | 17 | 0 | UHMK1 | Q8TAS1 | wp076_MFH.5.115.2 | MFH.5.115.2 | down | −0.877412538 | 0.000739818 |
| 17 | 17 | 0 | UHMK1 | Q8TAS1 | wp107_DB0646 | DB0646 | down | −0.326275612 | 0.000197215 |
| 17 | 17 | 0 | UHMK1 | Q8TAS1 | wp108_FMF.06.098.1 | FMF.06.098.1 | down | −0.387756183 | 0.001132872 |
| 17 | 17 | 0 | UHMK1 | Q8TAS1 | wp117_DB0646 | DB0646 | down | −0.472196356 | 0.000496664 |
| 17 | 17 | 0 | UHMK1 | Q8TAS1 | wp134_DB118430 | DB118430 | down | −0.494391888 | 0.000403498 |
| 17 | 17 | 0 | UHMK1 | Q8TAS1 | wp160_DB0646_2 hr | DB0646 | down | −0.446782013 | 0.001745867 |
| 17 | 17 | 0 | UHMK1 | Q8TAS1 | wp160_DB0646_4 hr | DB0646 | down | −0.755067772 | 0.00022456 |
| 17 | 17 | 0 | UHMK1 | Q8TAS1 | wp160_DB0646_8 hr | DB0646 | down | −0.519579946 | 0.000976154 |
| 17 | 17 | 0 | UHMK1 | Q8TAS1 | wp160_SK.3.91_2 hr | SK.3.91 | down | −0.414393003 | 0.002324385 |
| 17 | 17 | 0 | UHMK1 | Q8TAS1 | wp160_SK.3.91_4 hr | SK.3.91 | down | −0.39590066 | 0.002761275 |
| 17 | 17 | 0 | UHMK1 | Q8TAS1 | wp168_DB0661 | DB0661 | down | −0.416024416 | 0.00036577 |
| 17 | 17 | 0 | UHMK1 | Q8TAS1 | wp168_DB0662 | DB0662 | down | −0.39489636 | 0.000415042 |
| 17 | 17 | 0 | ULK3 | Q6PHR2-1 | wp016_SK.3.91_1 uM | SK.3.91 | down | −1.030299549 | 0.000327716 |
| 17 | 17 | 0 | ULK3 | Q6PHR2-1 | wp045_RSS0628 | RSS0628 | down | −1.086002132 | 6.87E−05 |
| 17 | 17 | 0 | ULK3 | Q6PHR2-1 | wp045_DB0614 | DB0614 | down | −0.946063719 | 0.000124611 |
| 17 | 12 | 5 | ULK3 | Q6PHR2-1 | wp103_SK.3.91 | SK.3.91 | down | −0.965060103 | 1.71E−05 |
| 17 | 12 | 5 | ULK3 | Q6PHR2-1 | wp105_SK.3.91 | SK.3.91 | down | −0.837016946 | 4.67E−06 |
| 17 | 12 | 5 | ULK3 | Q6PHR2-1 | wp117_DB0614 | DB0614 | down | −0.830655262 | 9.39E−06 |
| 17 | 12 | 5 | ULK3 | Q6PHR2-1 | wp131_TL12.186 | TL12.186 | down | −1.074088841 | 3.58E−07 |
| 17 | 12 | 5 | ULK3 | Q6PHR2-1 | wp133_SK.3.91_2 hr | SK.3.91 | down | −0.581704192 | 1.06E−05 |
| 17 | 12 | 5 | ULK3 | Q6PHR2-1 | wp133_SK.3.91_4 hr | SK.3.91 | down | −1.061827192 | 2.00E−07 |
| 17 | 12 | 5 | ULK3 | Q6PHR2-1 | wp137_TL12.186 | TL12.186 | down | −1.174201749 | 1.27E−06 |
| 17 | 12 | 5 | ULK3 | Q6PHR2-1 | wp137_TL13.97 | TL13.97 | down | −0.372942693 | 0.000607982 |
| 17 | 12 | 5 | ULK3 | Q6PHR2-1 | wp160_SK.3.91_2 hr | SK.3.91 | down | −0.502950712 | 0.001032839 |
| 17 | 12 | 5 | ULK3 | Q6PHR2-1 | wp160_SK.3.91_4 hr | SK.3.91 | down | −0.968858706 | 7.73E−05 |
| 17 | 12 | 5 | ULK3 | Q6PHR2-1 | wp172_SK.3.91 | SK.3.91 | down | −0.815073853 | 0.000159479 |
| 17 | 12 | 5 | BUB1B | O60566-3 | wp016_SK.3.91_1 uM | SK.3.91 | down | −0.468515198 | 0.00651015 |
| 17 | 16 | 1 | BUB1B | O60566-3 | wp076_MFH.5.126.1 | MFH.5.126.1 | down | −0.753184396 | 0.000111032 |
| 17 | 16 | 1 | BUB1B | O60566-3 | wp076_MFH.5.115.2 | MFH.5.115.2 | down | −0.501271164 | 0.000635032 |
| 17 | 16 | 1 | BUB1B | O60566-3 | wp103_SK.3.91 | SK.3.91 | down | −0.398257715 | 0.000382115 |
| 17 | 16 | 1 | BUB1B | O60566-3 | wp107_SK.3.91 | SK.3.91 | down | −0.5037114 | 9.42E−07 |
| 17 | 16 | 1 | BUB1B | O60566-3 | wp108_FMF.06.098.1 | FMF.06.098.1 | down | −0.442605366 | 3.98E−05 |
| 17 | 16 | 1 | BUB1B | O60566-3 | wp131_FMF.06.098.1 | FMF.06.098.1 | down | −0.423200889 | 3.11E−06 |
| 17 | 16 | 1 | BUB1B | O60566-3 | wp131_TL12.186 | TL12.186 | down | −0.541012077 | 9.92E−07 |
| 17 | 16 | 1 | BUB1B | O60566-3 | wp133_SK.3.91_2 hr | SK.3.91 | down | −0.419536761 | 1.43E−07 |
| 17 | 16 | 1 | BUB1B | O60566-3 | wp133_SK.3.91_1 hr | SK.3.91 | down | −0.408254529 | 2.69E−07 |
| 17 | 16 | 1 | BUB1B | O60566-3 | wp133_SK.3.91_4 hr | SK.3.91 | down | −0.587098061 | 4.60E−08 |
| 17 | 16 | 1 | BUB1B | O60566-3 | wp137_TL12.186 | TL12.186 | down | −0.551564952 | 7.36E−07 |
| 17 | 16 | 1 | BUB1B | O60566-3 | wp137_TL13.97 | TL13.97 | down | −0.331319577 | 1.22E−05 |
| 17 | 16 | 1 | CDK12 | Q9NYV4-1 | wp016_SK.3.91_1 uM | SK.3.91 | down | −0.932476301 | 6.26E−06 |
| 17 | 16 | 1 | CDK12 | Q9NYV4-1 | wp016_TL12.186 | TL12.186 | down | −0.918506014 | 6.68E−06 |
| 17 | 16 | 1 | CDK12 | Q9NYV4-1 | wp016_SK.3.93 | SK.3.93 | down | −0.955523016 | 5.63E−06 |
| 17 | 16 | 1 | CDK12 | Q9NYV4-1 | wp016_SK.3.91 | SK.3.91 | down | −1.134024977 | 2.67E−06 |
| 16 | 16 | 0 | CDK12 | Q9NYV4-1 | wp016_SK.3.87 | SK.3.87 | down | −0.507277292 | 8.68E−05 |
| 16 | 16 | 0 | CDK12 | Q9NYV4-1 | wp016_LT2.49 | LT2.49 | down | −0.868018267 | 8.54E−06 |
| 16 | 16 | 0 | CDK12 | Q9NYV4-1 | wp016_SK.3.89 | SK.3.89 | down | −0.523697652 | 7.57E−05 |
| 16 | 16 | 0 | CDK12 | Q9NYV4-1 | wp131_FMF.06.098.1 | FMF.06.098.1 | down | −0.57318899 | 6.12E−05 |
| 16 | 16 | 0 | CDK12 | Q9NYV4-1 | wp131_TL12.186 | TL12.186 | down | −1.402113057 | 5.31E−07 |
| 16 | 16 | 0 | CDK12 | Q9NYV4-1 | wp146_TMX.01.160 | TMX.01.160 | down | −0.511789235 | 0.00081768 |
| 16 | 16 | 0 | CDK12 | Q9NYV4-1 | wp146_TMX.02.138 | TMX.02.138 | down | −1.018084766 | 6.83E−05 |
| 16 | 16 | 0 | CDK12 | Q9NYV4-1 | wp146_TMX.02.174 | TMX.02.174 | down | −0.85621541 | 0.000128129 |
| 16 | 16 | 0 | CDK12 | Q9NYV4-1 | wp172_SK.3.91 | SK.3.91 | down | −1.234370924 | 2.32E−07 |
| 16 | 16 | 0 | MAP3K1 | Q13233 | wp056_SB1.G.187 | SB1.G.187 | down | −0.780886924 | 0.000405777 |
| 16 | 16 | 0 | MAP3K1 | Q13233 | wp056_WH.10417.099 | WH.10417.099 | down | −0.503374174 | 0.002318972 |
| 16 | 16 | 0 | MAP3K1 | Q13233 | wp076_MFH.5.126.1 | MFH.5.126.1 | down | −0.594680898 | 0.000835872 |
| 16 | 16 | 0 | MAP3K1 | Q13233 | wp076_MFH.5.115.2 | MFH.5.115.2 | down | −0.352786198 | 0.006850905 |
| 16 | 16 | 0 | MAP3K1 | Q13233 | wp103_SB1.G.187 | SB1.G.187 | down | −0.581548442 | 0.005444033 |
| 16 | 16 | 0 | MAP3K1 | Q13233 | wp105_SK.3.91 | SK.3.91 | down | −0.325762051 | 2.69E−05 |
| 16 | 16 | 0 | MAP3K1 | Q13233 | wp107_SB1.G.187 | SB1.G.187 | down | −0.66928383 | 0.00057506 |
| 16 | 16 | 0 | MAP3K1 | Q13233 | wp107_DB0646 | DB0646 | down | −0.376677958 | 0.005346687 |
| 16 | 16 | 0 | MAP3K1 | Q13233 | wp107_WH.10417.099 | WH.10417.099 | down | −0.467348691 | 0.002620435 |
| 16 | 16 | 0 | MAP3K1 | Q13233 | wp152_DB0646 | DB0646 | down | −0.613859513 | 3.79E−05 |
| 16 | 16 | 0 | MAP3K1 | Q13233 | wp160_DB0646_8 hr | DB0646 | down | −0.724076877 | 0.005523422 |
| 16 | 16 | 0 | MAP3K1 | Q13233 | wp168_DB0662 | DB0662 | down | −0.70867761 | 1.62E−05 |
| 16 | 16 | 0 | STK35 | Q8TDR2 | wp056_WH.10417.099 | WH.10417.099 | down | −2.072608521 | 1.80E−05 |
| 16 | 16 | 0 | STK35 | Q8TDR2 | wp103_WH.10417.099 | WH.10417.099 | down | −1.238730987 | 3.55E−05 |
| 16 | 16 | 0 | STK35 | Q8TDR2 | wp107_WH.10417.099 | WH.10417.099 | down | −1.953724427 | 6.60E−08 |
| 16 | 16 | 0 | STK35 | Q8TDR2 | wp108_FMF.06.098.1 | FMF.06.098.1 | down | −0.602894123 | 0.000238066 |
| 16 | 16 | 0 | STK35 | Q8TDR2 | wp114_WH.10414.099 | WH.10414.099 | down | −0.890200824 | 0.000126039 |
| 16 | 16 | 0 | STK35 | Q8TDR2 | wp114_FMF.06.098.1 | FMF.06.098.1 | down | −0.511584086 | 0.001128596 |
| 16 | 16 | 0 | STK35 | Q8TDR2 | wp117_RSS0680 | RSS0680 | down | −0.393739312 | 0.001072675 |
| 16 | 16 | 0 | STK35 | Q8TDR2 | wp131_FMF.06.098.1 | FMF.06.098.1 | down | −0.656046311 | 4.39E−06 |
| 16 | 16 | 0 | STK35 | Q8TDR2 | wp131_TL12.186 | TL12.186 | down | −0.413497179 | 3.88E−05 |
| 16 | 16 | 0 | STK35 | Q8TDR2 | wp137_TL12.186 | TL12.186 | down | −0.407892932 | 0.000864859 |
| 16 | 16 | 0 | STK35 | Q8TDR2 | wp146_TMX.02.174 | TMX.02.174 | down | −0.563128839 | 0.005457459 |
| 16 | 16 | 0 | STK35 | Q8TDR2 | wp152_DB1114 | DB1114 | down | −0.513429628 | 0.003005636 |
| 16 | 16 | 0 | STK35 | Q8TDR2 | wp168_DB1113 | DB1113 | down | −0.760633474 | 1.74E−05 |
| 16 | 16 | 0 | EPHA3 | P29320 | wp045_DB0646 | DB0646 | down | −0.533303694 | 0.000367323 |
| 16 | 16 | 0 | EPHA3 | P29320 | wp071_SB1.G.192.1 | SB1.G.192.1 | down | −1.175353382 | 2.38E−05 |
| 16 | 16 | 0 | EPHA3 | P29320 | wp081_SB1.G.192.1 | SB1.G.192.1 | down | −1.026988726 | 4.28E−06 |
| 16 | 16 | 0 | EPHA3 | P29320 | wp103_DB0646 | DB0646 | down | −0.371509586 | 0.001813727 |
| 16 | 16 | 0 | EPHA3 | P29320 | wp107_DB0646 | DB0646 | down | −0.434612048 | 3.14E−05 |
| 16 | 16 | 0 | EPHA3 | P29320 | wp117_DB0646 | DB0646 | down | −0.358679977 | 0.001552421 |
| 16 | 16 | 0 | EPHA3 | P29320 | wp131_SB1.G.192.1 | SB1.G.192.1 | down | −1.317896556 | 2.51E−07 |
| 16 | 16 | 0 | EPHA3 | P29320 | wp152_DB0646 | DB0646 | down | −0.387091145 | 6.66E−05 |
| 16 | 16 | 0 | EPHA3 | P29320 | wp152_DB1114 | DB1114 | down | −0.515857417 | 1.42E−05 |
| 16 | 16 | 0 | EPHA3 | P29320 | wp160_DB0646_4 hr | DB0646 | down | −0.58136166 | 0.000111449 |
| 16 | 16 | 0 | EPHA3 | P29320 | wp160_DB0646_8 hr | DB0646 | down | −0.552756275 | 0.000136336 |
| 16 | 16 | 0 | EPHA3 | P29320 | wp168_DB1113 | DB1113 | down | −0.418215191 | 7.43E−06 |
| 16 | 16 | 0 | MAPKAPK2 | P49137 | wp045_DB0646 | DB0646 | down | −0.433446555 | 0.000171724 |
| 16 | 16 | 0 | MAPKAPK2 | P49137 | wp105_DB0646 | DB0646 | down | −0.45620689 | 0.001074026 |
| 16 | 11 | 5 | MAPKAPK2 | P49137 | wp107_DB0646 | DB0646 | down | −0.373414778 | 4.96E−06 |
| 16 | 11 | 5 | MAPKAPK2 | P49137 | wp114_DB0646 | DB0646 | down | −0.491080616 | 0.000790887 |
| 16 | 11 | 5 | MAPKAPK2 | P49137 | wp117_DB0646 | DB0646 | down | −0.433327892 | 6.84E−06 |
| 16 | 11 | 5 | MAPKAPK2 | P49137 | wp152_DB0646 | DB0646 | down | −0.422325152 | 5.50E−06 |
| 16 | 11 | 5 | MAPKAPK2 | P49137 | wp152_DB1114 | DB1114 | down | −2.078285974 | 8.40E−10 |
| 16 | 11 | 5 | MAPKAPK2 | P49137 | wp160_DB0646_4 hr | DB0646 | down | −0.344359832 | 0.000143275 |
| 16 | 11 | 5 | MAPKAPK2 | P49137 | wp160_DB0646_8 hr | DB0646 | down | −0.622747229 | 1.33E−05 |
| 16 | 11 | 5 | MAPKAPK2 | P49137 | wp168_DB0662 | DB0662 | down | −0.330998745 | 0.000123122 |
| 16 | 11 | 5 | MAPKAPK2 | P49137 | wp168_DB1113 | DB1113 | down | −3.201001903 | 1.24E−08 |
| 16 | 11 | 5 | MAPKAPK2 | P49137 | wp172_DB0646 | DB0646 | down | −0.329357602 | 0.000815099 |
| 16 | 11 | 5 | PDK3 | Q15120-2 | wp056_SB1.G.187 | SB1.G.187 | down | −0.482669112 | 0.001356032 |
| 16 | 16 | 0 | PDK3 | Q15120-2 | wp056_ZNL.02.096 | ZNL.02.096 | down | −0.352754314 | 0.004545072 |
| 16 | 16 | 0 | PDK3 | Q15120-2 | wp081_INY.01.140.1 | INY.01.140.1 | down | −0.375504533 | 0.001449244 |
| 16 | 16 | 0 | PDK3 | Q15120-2 | wp084_BSJ.04.132 | BSJ.04.132 | down | −0.739656819 | 0.000143779 |
| 16 | 16 | 0 | PDK3 | Q15120-2 | wp084_BSJ.03.123 | BSJ.03.123 | down | −0.590818713 | 0.000734376 |
| 16 | 16 | 0 | PDK3 | Q15120-2 | wp137_DD.03.106 | DD.03.106 | down | −0.357055843 | 0.000423988 |
| 16 | 16 | 0 | PDK3 | Q15120-2 | wp147_DD.03.106_4 uM | DD.03.106 | down | −0.656447822 | 9.79E−05 |
| 16 | 16 | 0 | PDK3 | Q15120-2 | wp147_DD.03.107_1 uM | DD.03.107 | down | −0.511879083 | 0.000333312 |
| 16 | 16 | 0 | PDK3 | Q15120-2 | wp147_DD.03.107_2 uM | DD.03.107 | down | −0.54063924 | 0.000255299 |
| 16 | 16 | 0 | PDK3 | Q15120-2 | wp147_DD.03.107_4 uM | DD.03.107 | down | −1.004150659 | 1.15E−05 |
| 16 | 16 | 0 | RPS6KA1 | Q15418-2 | wp012_TL13.112 | TL13.112 | down | −0.787542007 | 6.63E−08 |
| 16 | 16 | 0 | RPS6KA1 | Q15418-2 | wp016_SK.3.91_1 uM | SK.3.91 | down | −0.414585809 | 0.000801929 |
| 16 | 16 | 0 | RPS6KA1 | Q15418-2 | wp016_SK.3.93 | SK.3.93 | down | −0.341332343 | 0.001779028 |
| 16 | 16 | 0 | RPS6KA1 | Q15418-2 | wp076_MFH.5.116.1 | MFH.5.116.1 | down | −0.575736511 | 8.40E−05 |
| 16 | 16 | 0 | RPS6KA1 | Q15418-2 | wp076_MFH.5.103.1 | MFH.5.103.1 | down | −1.582847006 | 9.87E−07 |
| 16 | 16 | 0 | RPS6KA1 | Q15418-2 | wp133_SK.3.91_4 hr | SK.3.91 | down | −0.359832675 | 5.27E−06 |
| 16 | 16 | 0 | RPS6KA1 | Q15418-2 | wp137_TL12.186 | TL12.186 | down | −0.451363647 | 0.000129304 |
| 15 | 15 | 0 | RPS6KA1 | Q15418-2 | wp137_TL13.97 | TL13.97 | down | −0.375731953 | 0.000338127 |
| 15 | 15 | 0 | RPS6KA1 | Q15418-2 | wp146_TMX.01.160 | TMX.01.160 | down | −0.431678658 | 0.000262048 |
| 15 | 15 | 0 | RPS6KA1 | Q15418-2 | wp146_TMX.02.138 | TMX.02.138 | down | −0.500996998 | 0.000152877 |
| 15 | 15 | 0 | RPS6KA1 | Q15418-2 | wp146_TMX.02.172 | TMX.02.172 | down | −1.350513677 | 4.10E−06 |
| 15 | 15 | 0 | RPS6KA1 | Q15418-2 | wp146_TMX.02.174 | TMX.02.174 | down | −0.663837702 | 5.50E−05 |
| 15 | 15 | 0 | TNK2 | Q07912-1 | wp108_FMF.06.098.1 | FMF.06.098.1 | down | −0.510831805 | 0.001018841 |
| 15 | 15 | 0 | TNK2 | Q07912-1 | wp131_FMF.06.098.1 | FMF.06.098.1 | down | −0.580479024 | 0.000112901 |
| 15 | 15 | 0 | TNK2 | Q07912-1 | wp131_TL12.186 | TL12.186 | down | −2.250711964 | 8.33E−08 |
| 15 | 15 | 0 | TNK2 | Q07912-1 | wp131_SB1.G.192.1 | SB1.G.192.1 | down | −1.488150654 | 9.34E−07 |
| 15 | 15 | 0 | TNK2 | Q07912-1 | wp133_SK.3.91_2 hr | SK.3.91 | down | −1.053325683 | 2.49E−05 |
| 15 | 15 | 0 | TNK2 | Q07912-1 | wp133_SK.3.91_4 hr | SK.3.91 | down | −2.260721542 | 3.94E−07 |
| 15 | 15 | 0 | TNK2 | Q07912-1 | wp134_DB118430 | DB118430 | down | −0.574992735 | 0.000461639 |
| 15 | 15 | 0 | TNK2 | Q07912-1 | wp137_TL12.186 | TL12.186 | down | −1.575283562 | 1.65E−05 |
| 15 | 15 | 0 | TNK2 | Q07912-1 | wp137_TL13.97 | TL13.97 | down | −1.970990674 | 4.83E−06 |
| 15 | 15 | 0 | TNK2 | Q07912-1 | wp168_DB0661 | DB0661 | down | −0.387310202 | 0.009737031 |
| 15 | 12 | 3 | TNK2 | Q07912-1 | wp168_DB0662 | DB0662 | down | −0.694267813 | 0.001062797 |
| 15 | 12 | 3 | TNK2 | Q07912-1 | wp168_DB1113 | DB1113 | down | −0.535090979 | 0.003439972 |
| 15 | 12 | 3 | ABL1 | P00519-2 | wp016_SK.3.91_1 uM | SK.3.91 | down | −0.387454675 | 0.000949779 |
| 15 | 12 | 3 | ABL1 | P00519-2 | wp056_SB1.G.187 | SB1.G.187 | down | −0.517297597 | 0.000208254 |
| 15 | 12 | 3 | ABL1 | P00519-2 | wp071_SB1.G.192.1 | SB1.G.192.1 | down | −1.416782725 | 0.000261116 |
| 15 | 12 | 3 | ABL1 | P00519-2 | wp071_SB1.G.181 | SB1.G.181 | down | −1.734664813 | 0.000100211 |
| 15 | 12 | 3 | ABL1 | P00519-2 | wp071_SB1.G.200 | SB1.G.200 | down | −1.646322769 | 0.0001285 |
| 15 | 12 | 3 | ABL1 | P00519-2 | wp081_SB1.G.192.1 | SB1.G.192.1 | down | −1.189021162 | 9.30E−08 |
| 15 | 12 | 3 | ABL1 | P00519-2 | wp103_SB1.G.187 | SB1.G.187 | down | −0.754002472 | 0.000802195 |
| 15 | 12 | 3 | ABL1 | P00519-2 | wp105_SK.3.91 | SK.3.91 | down | −0.332980147 | 3.57E−05 |
| 15 | 12 | 3 | ABL1 | P00519-2 | wp107_SK.3.91 | SK.3.91 | down | −0.47106163 | 1.17E−05 |
| 15 | 12 | 3 | ABL1 | P00519-2 | wp107_SB1.G.187 | SB1.G.187 | down | −0.533929352 | 2.38E−06 |
| 15 | 15 | 0 | ABL1 | P00519-2 | wp133_SK.3.91_4 hr | SK.3.91 | down | −0.380110304 | 7.66E−06 |
| 15 | 15 | 0 | CDK1 | P06493 | wp104_TMX.01.160.1_0.25 | TMX.01.160.1 | down | −0.793607715 | 3.43E−06 |
| 15 | 15 | 0 | CDK1 | P06493 | wp104_TMX.01.160.1_1 | TMX.01.160.1 | down | −0.941297573 | 1.44E−06 |
| 15 | 15 | 0 | CDK1 | P06493 | wp117_RSS0680 | RSS0680 | down | −0.358657158 | 2.73E−05 |
| 15 | 15 | 0 | CDK1 | P06493 | wp131_FMF.06.098.1 | FMF.06.098.1 | down | −0.353527874 | 0.000160916 |
| 15 | 15 | 0 | CDK1 | P06493 | wp146_TMX.01.160 | TMX.01.160 | down | −1.422737961 | 0.000415602 |
| 15 | 15 | 0 | CDK1 | P06493 | wp146_TMX.02.138 | TMX.02.138 | down | −1.641244665 | 0.000248328 |
| 15 | 15 | 0 | CDK1 | P06493 | wp146_TMX.02.172 | TMX.02.172 | down | −1.302465705 | 0.000570603 |
| 15 | 15 | 0 | CDK1 | P06493 | wp146_TMX.02.174 | TMX.02.174 | down | −1.629011611 | 0.000255131 |
| 15 | 15 | 0 | CDK1 | P06493 | wp147_DD.03.106_2 uM | DD.03.106 | down | −0.501516658 | 2.03E−06 |
| 15 | 15 | 0 | CDK1 | P06493 | wp147_DD.03.106_4 uM | DD.03.106 | down | −0.541605257 | 1.37E−06 |
| 15 | 15 | 0 | CSNK1D | P48730-2 | wp056_WH.10417.099 | WH.10417.099 | down | −1.455411131 | 0.000213519 |
| 15 | 15 | 0 | CSNK1D | P48730-2 | wp076_MFH.5.126.1 | MFH.5.126.1 | down | −0.920078809 | 0.009009358 |
| 15 | 15 | 0 | CSNK1D | P48730-2 | wp103_WH.10417.099 | WH.10417.099 | down | −1.300682413 | 1.26E−05 |
| 15 | 15 | 0 | CSNK1D | P48730-2 | wp107_WH.10417.099 | WH.10417.099 | down | −1.248493519 | 3.03E−05 |
| 15 | 15 | 0 | CSNK1D | P48730-2 | wp108_FMF.06.098.1 | FMF.06.098.1 | down | −0.38124569 | 0.006668815 |
| 15 | 15 | 0 | CSNK1D | P48730-2 | wp114_SK.3.91 | SK.3.91 | down | −0.399085737 | 0.007529838 |
| 15 | 15 | 0 | CSNK1D | P48730-2 | wp114_WH.10414.099 | WH.10414.099 | down | −0.618735506 | 0.001463406 |
| 15 | 15 | 0 | CSNK1D | P48730-2 | wp114_FMF.06.098.1 | FMF.06.098.1 | down | −0.403945403 | 0.007209686 |
| 15 | 15 | 0 | CSNK1D | P48730-2 | wp137_TL12.186 | TL12.186 | down | −0.47211173 | 0.000216556 |
| 15 | 15 | 0 | CSNK1D | P48730-2 | wp160_SK.3.91_4 hr | SK.3.91 | down | −0.337298188 | 0.001038545 |
| 15 | 15 | 0 | CSNK1D | P48730-2 | wp172_WH.10417.099 | WH.10417.099 | down | −0.640085217 | 0.004363956 |
| 15 | 15 | 0 | MAP3K20 | Q9NYL2 | wp045_DB0646 | DB0646 | down | −0.424840116 | 0.004402171 |
| 15 | 15 | 0 | MAP3K20 | Q9NYL2 | wp107_DB0646 | DB0646 | down | −0.530029375 | 0.000347567 |
| 15 | 15 | 0 | MAP3K20 | Q9NYL2 | wp147_DD.03.106_2 uM | DD.03.106 | down | −0.407941446 | 2.63E−05 |
| 15 | 15 | 0 | MAP3K20 | Q9NYL2 | wp152_DB0646 | DB0646 | down | −0.655594631 | 3.80E−06 |
| 15 | 15 | 0 | MAP3K20 | Q9NYL2 | wp152_DB1114 | DB1114 | down | −0.758471817 | 1.71E−06 |
| 15 | 15 | 0 | MAP3K20 | Q9NYL2 | wp160_DB0646_2 hr | DB0646 | down | −0.329287486 | 0.00085226 |
| 15 | 15 | 0 | MAP3K20 | Q9NYL2 | wp160_DB0646_4 hr | DB0646 | down | −0.619193447 | 6.98E−05 |
| 15 | 15 | 0 | MAP3K20 | Q9NYL2 | wp160_DB0646_8 hr | DB0646 | down | −0.919697514 | 1.42E−05 |
| 15 | 15 | 0 | MAP3K20 | Q9NYL2 | wp168_DB0662 | DB0662 | down | −0.737049617 | 4.97E−07 |
| 15 | 15 | 0 | MAP3K20 | Q9NYL2 | wp168_DB1113 | DB1113 | down | −1.380022133 | 9.21E−09 |
| 15 | 15 | 0 | MAP3K20 | Q9NYL2 | wp172_DB0646 | DB0646 | down | −0.386368211 | 0.001162434 |
| 15 | 15 | 0 | MAPK14 | Q16539-2 | wp047_DD.02.198 | DD.02.198 | down | −0.616965374 | 0.002152705 |
| 15 | 15 | 0 | MAPK14 | Q16539-2 | wp056_SB1.G.187 | SB1.G.187 | down | −0.801943662 | 0.009826934 |
| 15 | 15 | 0 | MAPK14 | Q16539-2 | wp081_TL13.178 | TL13.178 | down | −0.33425976 | 0.004586008 |
| 15 | 15 | 0 | MAPK14 | Q16539-2 | wp081_TL13.150 | TL13.150 | down | −0.502483821 | 0.000647401 |
| 15 | 15 | 0 | MAPK14 | Q16539-2 | wp105_SB1.G.187 | SB1.G.187 | down | −0.908361803 | 0.000548458 |
| 15 | 15 | 0 | MAPK14 | Q16539-2 | wp105_DB0646 | DB0646 | down | −0.582188568 | 0.004381136 |
| 15 | 15 | 0 | MAPK14 | Q16539-2 | wp117_DB0646 | DB0646 | down | −0.64360505 | 0.003393057 |
| 15 | 15 | 0 | MAPK14 | Q16539-2 | wp160_DB0646_4 hr | DB0646 | down | −0.705078364 | 0.001463 |
| 15 | 15 | 0 | MAPK14 | Q16539-2 | wp160_DB0646_8 hr | DB0646 | down | −1.091890719 | 0.000264487 |
| 15 | 15 | 0 | MAPK14 | Q16539-2 | wp168_DB0662 | DB0662 | down | −1.339524939 | 5.07E−05 |
| 15 | 15 | 0 | MAPK14 | Q16539-2 | wp168_DB1113 | DB1113 | down | −1.587786528 | 1.72E−05 |
| 15 | 15 | 0 | MAPK8 | P45983-4 | wp045_DB0646 | DB0646 | down | −0.977410744 | 5.81E−06 |
| 15 | 15 | 0 | MAPK8 | P45983-4 | wp045_DB0614 | DB0614 | down | −0.688215384 | 2.69E−05 |
| 15 | 15 | 0 | MAPK8 | P45983-4 | wp104_TMX.01.160.1_0.25 | TMX.01.160.1 | down | −0.333838422 | 0.002250517 |
| 14 | 14 | 0 | MAPK8 | P45983-4 | wp104_TMX.01.160.1_1 | TMX.01.160.1 | down | −0.476004038 | 0.000427745 |
| 14 | 14 | 0 | MAPK8 | P45983-4 | wp146_TMX.01.160 | TMX.01.160 | down | −2.112406584 | 3.16E−05 |
| 14 | 14 | 0 | MAPK8 | P45983-4 | wp146_TMX.02.138 | TMX.02.138 | down | −0.98125861 | 0.000509692 |
| 14 | 14 | 0 | MAPK8 | P45983-4 | wp146_TMX.02.172 | TMX.02.172 | down | −0.55241107 | 0.003851962 |
| 14 | 14 | 0 | MAPK8 | P45983-4 | wp146_TMX.02.174 | TMX.02.174 | down | −0.776138271 | 0.00117603 |
| 14 | 14 | 0 | MAPK8 | P45983-4 | wp168_DB0661 | DB0661 | down | −0.503349296 | 6.27E−06 |
| 14 | 14 | 0 | MAPK8 | P45983-4 | wp168_DB0662 | DB0662 | down | −0.896067771 | 4.14E−08 |
| 14 | 14 | 0 | MAPK8 | P45983-4 | wp168_DB1113 | DB1113 | down | −0.364156457 | 6.70E−06 |
| 14 | 14 | 0 | FYN | P06241-2 | wp047_DD.03.119 | DD.03.119 | down | −0.866895852 | 9.33E−05 |
| 14 | 14 | 0 | FYN | P06241-2 | wp071_SB1.G.192.1 | SB1.G.192.1 | down | −1.594948887 | 5.18E−06 |
| 14 | 14 | 0 | FYN | P06241-2 | wp071_SB1.G.181 | SB1.G.181 | down | −2.275905156 | 9.12E−07 |
| 14 | 14 | 0 | FYN | P06241-2 | wp071_SB1.G.200 | SB1.G.200 | down | −1.777318755 | 3.05E−06 |
| 14 | 14 | 0 | FYN | P06241-2 | wp081_SB1.G.192.1 | SB1.G.192.1 | down | −0.999408605 | 5.46E−05 |
| 14 | 14 | 0 | FYN | P06241-2 | wp107_SB1.G.187 | SB1.G.187 | down | −0.533361475 | 0.000280157 |
| 14 | 14 | 0 | FYN | P06241-2 | wp107_DB0646 | DB0646 | down | −0.399883512 | 0.000608967 |
| 14 | 14 | 0 | FYN | P06241-2 | wp131_SB1.G.192.1 | SB1.G.192.1 | down | −0.991780043 | 1.65E−07 |
| 14 | 14 | 0 | FYN | P06241-2 | wp160_DB0646_4 hr | DB0646 | down | −0.392183452 | 0.000613497 |
| 14 | 14 | 0 | FYN | P06241-2 | wp168_DB0662 | DB0662 | down | −0.563593214 | 0.009551282 |
| 14 | 14 | 0 | GSK3A | P49840 | wp017_WH.9533.153 | WH.9533.153 | down | −0.483326863 | 0.009803713 |
| 14 | 14 | 0 | GSK3A | P49840 | wp056_SB1.G.190 | SB1.G.190 | down | −0.889389103 | 0.000104102 |
| 14 | 14 | 0 | GSK3A | P49840 | wp056_WH.10417.099 | WH.10417.099 | down | −0.516857435 | 0.00094025 |
| 14 | 14 | 0 | GSK3A | P49840 | wp068_FMF.04.147.1 | FMF.04.147.1 | down | −0.534601985 | 0.000632196 |
| 14 | 14 | 0 | GSK3A | P49840 | wp076_MFH.5.126.1 | MFH.5.126.1 | down | −0.753103119 | 0.000520368 |
| 14 | 14 | 0 | GSK3A | P49840 | wp103_WH.10417.099 | WH.10417.099 | down | −0.442971723 | 0.000910271 |
| 14 | 14 | 0 | GSK3A | P49840 | wp105_WH.10417.099 | WH.10417.099 | down | −0.449904206 | 1.74E−05 |
| 14 | 14 | 0 | GSK3A | P49840 | wp107_WH.10417.099 | WH.10417.099 | down | −0.469694222 | 0.009939903 |
| 14 | 14 | 0 | GSK3A | P49840 | wp146_TMX.02.138 | TMX.02.138 | down | −0.822459901 | 0.000587383 |
| 14 | 14 | 0 | GSK3A | P49840 | wp146_TMX.02.174 | TMX.02.174 | down | −0.71334143 | 0.000975961 |
| 14 | 13 | 1 | GSK3B | P49841-2 | wp017_WH.9533.153 | WH.9533.153 | down | −1.316656528 | 1.80E−06 |
| 14 | 13 | 1 | GSK3B | P49841-2 | wp056_SB1.G.190 | SB1.G.190 | down | −0.630955865 | 0.001519593 |
| 14 | 13 | 1 | GSK3B | P49841-2 | wp056_WH.10417.099 | WH.10417.099 | down | −1.293577666 | 8.35E−05 |
| 14 | 13 | 1 | GSK3B | P49841-2 | wp068_FMF.04.147.1 | FMF.04.147.1 | down | −0.621840447 | 4.33E−05 |
| 14 | 13 | 1 | GSK3B | P49841-2 | wp076_MFH.5.126.1 | MFH.5.126.1 | down | −0.962949622 | 0.000146267 |
| 14 | 13 | 1 | GSK3B | P49841-2 | wp103_WH.10417.099 | WH.10417.099 | down | −1.442243756 | 4.93E−05 |
| 14 | 13 | 1 | GSK3B | P49841-2 | wp105_WH.10417.099 | WH.10417.099 | down | −0.840307264 | 2.07E−07 |
| 14 | 13 | 1 | GSK3B | P49841-2 | wp107_WH.10417.099 | WH.10417.099 | down | −1.445674546 | 4.47E−08 |
| 14 | 13 | 1 | GSK3B | P49841-2 | wp146_TMX.02.138 | TMX.02.138 | down | −0.60400236 | 0.002622533 |
| 14 | 13 | 1 | GSK3B | P49841-2 | wp146_TMX.02.174 | TMX.02.174 | down | −0.647203521 | 0.002061587 |
| 14 | 13 | 1 | MAP4K3 | Q81VH8 | wp105_SK.3.91 | SK.3.91 | down | −0.571019415 | 2.31E−06 |
| 14 | 13 | 1 | MAP4K3 | Q81VH8 | wp107_SK.3.91 | SK.3.91 | down | −0.845615749 | 0.000177347 |
| 14 | 13 | 1 | MAP4K3 | Q81VH8 | wp108_FMF.06.098.1 | FMF.06.098.1 | down | −0.814010842 | 0.000754522 |
| 14 | 14 | 0 | MAP4K3 | Q81VH8 | wp114_FMF.06.098.1 | FMF.06.098.1 | down | −0.414612938 | 0.003170227 |
| 14 | 14 | 0 | MAP4K3 | Q81VH8 | wp131_FMF.06.098.1 | FMF.06.098.1 | down | −0.789799606 | 1.04E−05 |
| 14 | 14 | 0 | MAP4K3 | Q81VH8 | wp131_TL12.186 | TL12.186 | down | −0.627493538 | 8.87E−05 |
| 14 | 14 | 0 | MAP4K3 | Q81VH8 | wp133_SK.3.91_4 hr | SK.3.91 | down | −0.632122593 | 0.000219949 |
| 14 | 14 | 0 | MAP4K3 | Q81VH8 | wp137_TL12.186 | TL12.186 | down | −0.399698169 | 0.002219622 |
| 14 | 14 | 0 | MAP4K3 | Q81VH8 | wp168_DB1113 | DB1113 | down | −1.385071654 | 3.11E−06 |
| 14 | 14 | 0 | MAP4K3 | Q81VH8 | wp172_SK.3.91 | SK.3.91 | down | −0.79619196 | 0.001838496 |
| 14 | 14 | 0 | PAK4 | O96013-1 | wp056_ZNL.02.096 | ZNL.02.096 | down | −0.633965883 | 0.005875861 |
| 14 | 14 | 0 | PAK4 | O96013-1 | wp081_ZNL.02.096 | ZNL.02.096 | down | −0.398876643 | 0.000272342 |
| 14 | 14 | 0 | PAK4 | O96013-1 | wp103_WH.10417.099 | WH.10417.099 | down | −0.370467389 | 0.002660898 |
| 14 | 14 | 0 | PAK4 | O96013-1 | wp105_WH.10417.099 | WH.10417.099 | down | −0.347785658 | 0.000127826 |
| 14 | 14 | 0 | PAK4 | O96013-1 | wp106_ZNL.02.096_4 hr | ZNL.02.096 | down | −0.576815619 | 1.43E−05 |
| 14 | 14 | 0 | PAK4 | O96013-1 | wp106_ZNL.02.096_2 hr | ZNL.02.096 | down | −0.423812359 | 2.09E−05 |
| 14 | 14 | 0 | PAK4 | O96013-1 | wp107_WH.10417.099 | WH.10417.099 | down | −0.479728299 | 0.000146297 |
| 13 | 13 | 0 | PAK4 | O96013-1 | wp118_ZNL.02.096 | ZNL.02.096 | down | −0.573200955 | 4.67E−05 |
| 13 | 13 | 0 | PAK4 | O96013-1 | wp168_DB0662 | DB0662 | down | −1.339812159 | 1.78E−05 |
| 13 | 13 | 0 | PAK4 | O96013-1 | wp195_BJG.04.203 | BJG.04.203 | down | −0.748648539 | 3.10E−06 |
| 13 | 13 | 0 | PRAG1 | Q86YV5 | wp107_SK.3.91 | SK.3.91 | down | −0.369195382 | 0.005319005 |
| 13 | 13 | 0 | PRAG1 | Q86YV5 | wp131_TL12.186 | TL12.186 | down | −0.424252207 | 3.70E−05 |
| 13 | 13 | 0 | PRAG1 | Q86YV5 | wp133_SK.3.91_2 hr | SK.3.91 | down | −0.366731323 | 0.000530451 |
| 13 | 13 | 0 | PRAG1 | Q86YV5 | wp133_SK.3.91_4 hr | SK.3.91 | down | −0.435169124 | 0.001672272 |
| 13 | 13 | 0 | PRAG1 | Q86YV5 | wp137_TL12.186 | TL12.186 | down | −0.345918831 | 0.00513466 |
| 13 | 13 | 0 | PTK2 | Q05397 | wp011_TL13.12 | TL13.12 | down | −1.365223299 | 3.12E−09 |
| 13 | 13 | 0 | PTK2 | Q05397 | wp105_SK.3.91 | SK.3.91 | down | −2.957603299 | 1.91E−10 |
| 13 | 13 | 0 | PTK2 | Q05397 | wp105_DB0646 | DB0646 | down | −0.557540293 | 1.01E−06 |
| 13 | 13 | 0 | PTK2 | Q05397 | wp114_DB0646 | DB0646 | down | −0.553848593 | 0.000191617 |
| 13 | 13 | 0 | PTK2 | Q05397 | wp114_SK.3.91 | SK.3.91 | down | −1.684347503 | 2.13E−06 |
| 13 | 12 | 1 | PTK2 | Q05397 | wp114_FMF.06.098.1 | FMF.06.098.1 | down | −0.947749481 | 2.20E−05 |
| 13 | 12 | 1 | PTK2 | Q05397 | wp146_TMX.02.172 | TMX.02.172 | down | −0.738108155 | 0.001153818 |
| 13 | 12 | 1 | PTK2 | Q05397 | wp146_TMX.02.174 | TMX.02.174 | down | −0.819784544 | 0.000794622 |
| 13 | 12 | 1 | PTK2 | Q05397 | wp172_DB0646 | DB0646 | down | −0.804717769 | 1.97E−06 |
| 13 | 12 | 1 | PTK2 | Q05397 | wp172_SK.3.91 | SK.3.91 | down | −2.688591599 | 8.60E−10 |
| 13 | 12 | 1 | RIOK2 | Q9BVS4 | wp016_SK.3.91_1 uM | SK.3.91 | down | −0.631874635 | 5.10E−05 |
| 13 | 12 | 1 | RIOK2 | Q9BVS4 | wp076_MFH.5.126.1 | MFH.5.126.1 | down | −0.78740412 | 0.000106997 |
| 13 | 12 | 1 | RIOK2 | Q9BVS4 | wp076_MFH.5.115.2 | MFH.5.115.2 | down | −0.806725691 | 9.63E−05 |
| 13 | 12 | 1 | RIOK2 | Q9BVS4 | wp103_SK.3.91 | SK.3.91 | down | −0.831103789 | 0.00010885 |
| 13 | 12 | 1 | RIOK2 | Q9BVS4 | wp105_SK.3.91 | SK.3.91 | down | −0.482035637 | 3.56E−05 |
| 13 | 12 | 1 | RIOK2 | Q9BVS4 | wp107_SK.3.91 | SK.3.91 | down | −0.73932534 | 1.88E−06 |
| 13 | 12 | 1 | RIOK2 | Q9BVS4 | wp131_TL12.186 | TL12.186 | down | −0.913422529 | 5.33E−08 |
| 13 | 13 | 0 | RIOK2 | Q9BVS4 | wp133_SK.3.91_4 hr | SK.3.91 | down | −0.647593588 | 1.38E−07 |
| 13 | 13 | 0 | RIOK2 | Q9BVS4 | wp137_TL12.186 | TL12.186 | down | −0.772751831 | 1.36E−07 |
| 13 | 13 | 0 | RIOK2 | Q9BVS4 | wp160_SK.3.91_4 hr | SK.3.91 | down | −0.441640477 | 0.000162584 |
| 13 | 13 | 0 | BMP2K | Q9NSY1 | wp016_SK.3.93 | SK.3.93 | down | −0.457537383 | 0.005483644 |
| 13 | 13 | 0 | BMP2K | Q9NSY1 | wp016_SK.3.91 | SK.3.91 | down | −0.47056948 | 0.004921909 |
| 13 | 13 | 0 | BMP2K | Q9NSY1 | wp016_LT2.49 | LT2.49 | down | −0.5079017 | 0.003656456 |
| 13 | 13 | 0 | BMP2K | Q9NSY1 | wp045_DB0614 | DB0614 | down | −0.390930471 | 0.007338111 |
| 13 | 13 | 0 | BMP2K | Q9NSY1 | wp056_SB1.G.190 | SB1.G.190 | down | −0.350761385 | 0.002143819 |
| 13 | 13 | 0 | BMP2K | Q9NSY1 | wp071_JWG.118 | JWG.118 | down | −0.352810142 | 0.002520851 |
| 13 | 13 | 0 | BMP2K | Q9NSY1 | wp105_SK.3.91 | SK.3.91 | down | −0.404317151 | 0.003233663 |
| 13 | 13 | 0 | BMP2K | Q9NSY1 | wp134_BSJ.04.178 | BSJ.04.178 | down | −0.692900154 | 8.93E−07 |
| 13 | 13 | 0 | BMP2K | Q9NSY1 | wp195_BJG.04.203 | BJG.04.203 | down | −0.461928895 | 5.46E−05 |
| 13 | 13 | 0 | LATS1 | O95835 | wp071_SB1.G.181 | SB1.G.181 | down | −1.27333964 | 1.84E−05 |
| 12 | 12 | 0 | LATS1 | O95835 | wp071_SB1.G.200 | SB1.G.200 | down | −1.403246076 | 1.15E−05 |
| 12 | 12 | 0 | LATS1 | O95835 | wp105_SK.3.91 | SK.3.91 | down | −0.333496548 | 9.48E−05 |
| 12 | 12 | 0 | LATS1 | O95835 | wp107_SK.3.91 | SK.3.91 | down | −0.460912978 | 1.01E−05 |
| 12 | 12 | 0 | LATS1 | O95835 | wp117_RSS0680 | RSS0680 | down | −0.414250893 | 6.01E−05 |
| 12 | 12 | 0 | LATS1 | O95835 | wp160_DB0646_8 hr | DB0646 | down | −0.338194257 | 0.005180106 |
| 12 | 12 | 0 | LATS1 | O95835 | wp160_SK.3.91_4 hr | SK.3.91 | down | −0.346305709 | 0.004749448 |
| 12 | 12 | 0 | LATS1 | O95835 | wp195_BJG.04.203 | BJG.04.203 | down | −0.391906941 | 0.002751905 |
| 12 | 12 | 0 | LATS1 | O95835 | wp195_INY.04.125.01 | INY.04.125.01 | down | −0.417603825 | 0.002031876 |
| 12 | 12 | 0 | CDK11B | P21127-1 | wp045_DB0646 | DB0646 | down | −0.455494716 | 0.001786582 |
| 12 | 12 | 0 | CDK11B | P21127-1 | wp105_DB0646 | DB0646 | down | −0.354247734 | 0.006982547 |
| 12 | 12 | 0 | CDK11B | P21127-1 | wp117_DB0646 | DB0646 | down | −0.46945905 | 0.000984075 |
| 12 | 12 | 0 | CDK11B | P21127-1 | wp160_DB0646_4 hr | DB0646 | down | −0.512194379 | 0.000340456 |
| 12 | 12 | 0 | CDK11B | P21127-1 | wp160_DB0646_8 hr | DB0646 | down | −0.644012321 | 0.000137054 |
| 12 | 12 | 0 | CDK11B | P21127-1 | wp168_DB0662 | DB0662 | down | −0.594614948 | 1.21E−06 |
| 12 | 12 | 0 | CDK11B | P21127-1 | wp168_DB1113 | DB1113 | down | −0.87357786 | 1.14E−07 |
| 12 | 12 | 0 | CDK11B | P21127-1 | wp172_DB0646 | DB0646 | down | −0.462415612 | 0.000250528 |
| 12 | 12 | 0 | CSNK1E | P49674 | wp056_WH.10417.099 | WH.10417.099 | down | −1.59128646 | 0.002294281 |
| 12 | 12 | 0 | CSNK1E | P49674 | wp071_SB1.G.181 | SB1.G.181 | down | −1.189416881 | 0.000272879 |
| 12 | 12 | 0 | CSNK1E | P49674 | wp071_SB1.G.200 | SB1.G.200 | down | −0.759976078 | 0.002103984 |
| 12 | 12 | 0 | CSNK1E | P49674 | wp103_WH.10417.099 | WH.10417.099 | down | −1.306144177 | 2.73E−05 |
| 12 | 12 | 0 | CSNK1E | P49674 | wp105_WH.10417.099 | WH.10417.099 | down | −0.830541147 | 6.93E−06 |
| 12 | 12 | 0 | CSNK1E | P49674 | wp107_WH.10417.099 | WH.10417.099 | down | −2.192179017 | 2.18E−06 |
| 12 | 12 | 0 | CSNK1E | P49674 | wp137_TL12.186 | TL12.186 | down | −0.441137532 | 0.004534936 |
| 12 | 12 | 0 | CSNK1E | P49674 | wp172_WH.10417.099 | WH.10417.099 | down | −1.163808628 | 4.84E−07 |
| 12 | 12 | 0 | LCK | P06239-1 | wp081_TL13.150 | TL13.150 | down | −0.712918258 | 6.32E−07 |
| 12 | 12 | 0 | LCK | P06239-1 | wp081_SB1.G.192.1 | SB1.G.192.1 | down | −1.748053014 | 4.51E−09 |
| 12 | 12 | 0 | LCK | P06239-1 | wp160_DB0646_4 hr | DB0646 | down | −0.457544313 | 3.68E−05 |
| 12 | 12 | 0 | LCK | P06239-1 | wp160_DB0646_8 hr | DB0646 | down | −0.538584008 | 1.91E−05 |
| 12 | 12 | 0 | LCK | P06239-1 | wp160_SK.3.91_2 hr | SK.3.91 | down | −0.462668306 | 3.52E−05 |
| 12 | 12 | 0 | LCK | P06239-1 | wp160_SK.3.91_4 hr | SK.3.91 | down | −1.173450663 | 8.16E−07 |
| 12 | 12 | 0 | LCK | P06239-1 | wp168_DB0661 | DB0661 | down | −0.471746877 | 1.54E−06 |
| 12 | 12 | 0 | LCK | P06239-1 | wp168_DB0662 | DB0662 | down | −0.907833099 | 2.32E−07 |
| 12 | 12 | 0 | MAPK6 | Q16659 | wp080_RSS0680 | RSS0680 | down | −0.503315158 | 0.00545224 |
| 12 | 12 | 0 | MAPK6 | Q16659 | wp103_SK.3.91 | SK.3.91 | down | −1.525487028 | 0.000120586 |
| 12 | 12 | 0 | MAPK6 | Q16659 | wp107_SK.3.91 | SK.3.91 | down | −1.344982477 | 2.73E−05 |
| 12 | 12 | 0 | MAPKG | Q16659 | wp118_INY.03.041.01 | INY.03.041.01 | down | −0.618325187 | 0.00406575 |
| 11 | 10 | 1 | MAPK6 | Q16659 | wp131_FMF.06.098.1 | FMF.06.098.1 | down | −0.41225707 | 0.009597523 |
| 11 | 10 | 1 | MAPK6 | Q16659 | wp131_TL12.186 | TL12.186 | down | −1.488008855 | 2.12E−05 |
| 11 | 10 | 1 | MAPK6 | Q16659 | wp133_SK.3.91_2 hr | SK.3.91 | down | −0.82527864 | 0.000115934 |
| 11 | 10 | 1 | MAPK6 | Q16659 | wp133_SK.3.91_4 hr | SK.3.91 | down | −1.613633847 | 3.18E−06 |
| 11 | 10 | 1 | NEK2 | P51955 | wp103_SK.3.91 | SK.3.91 | down | −0.456432837 | 0.003152325 |
| 11 | 10 | 1 | NEK2 | P51955 | wp105_SK.3.91 | SK.3.91 | down | −0.390755494 | 0.006168287 |
| 11 | 10 | 1 | NEK2 | P51955 | wp107_SK.3.91 | SK.3.91 | down | −0.636840253 | 0.000247821 |
| 11 | 10 | 1 | NEK2 | P51955 | wp131_TL12.186 | TL12.186 | down | −0.378292524 | 0.005170447 |
| 11 | 10 | 1 | NEK2 | P51955 | wp133_SK.3.91_2 hr | SK.3.91 | down | −0.441130124 | 0.001344277 |
| 11 | 10 | 1 | NEK2 | P51955 | wp133_SK.3.91_1 hr | SK.3.91 | down | −0.439675559 | 0.000666456 |
| 11 | 11 | 0 | NEK2 | P51955 | wp133_SK.3.91_4 hr | SK.3.91 | down | −0.730292907 | 5.51E−05 |
| 11 | 11 | 0 | NEK2 | P51955 | wp160_SK.3.91_4 hr | SK.3.91 | down | −0.42942459 | 0.007247754 |
| 11 | 11 | 0 | NEK9 | Q8TD19 | wp045_RSS0628 | RSS0628 | down | −2.437653972 | 4.95E−09 |
| 11 | 11 | 0 | NEK9 | Q8TD19 | wp045_DB0614 | DB0614 | down | −2.739791603 | 2.96E−09 |
| 11 | 11 | 0 | NEK9 | Q8TD19 | wp080_RSS0680 | RSS0680 | down | −1.276980551 | 7.14E−08 |
| 11 | 11 | 0 | NEK9 | Q8TD19 | wp117_RSS0680 | RSS0680 | down | −1.483192034 | 3.04E−08 |
| 11 | 11 | 0 | NEK9 | Q8TD19 | wp117_DB0614 | DB0614 | down | −3.00640942 | 9.54E−08 |
| 11 | 11 | 0 | NEK9 | Q8TD19 | wp146_TMX.02.174 | TMX.02.174 | down | −0.362557079 | 0.006629791 |
| 11 | 11 | 0 | NEK9 | Q8TD19 | wp195_BJG.04.203 | BJG.04.203 | down | −1.42496418 | 3.32E−09 |
| 11 | 11 | 0 | NEK9 | Q8TD19 | wp195_JWG.123 | JWG.123 | down | −1.078654542 | 1.58E−08 |
| 11 | 11 | 0 | NLK | Q9UBE8 | wp071_SB1.G.192.1 | SB1.G.192.1 | down | −0.557841692 | 0.003520898 |
| 11 | 11 | 0 | NLK | Q9UBE8 | wp081_SB1.G.192.1 | SB1.G.192.1 | down | −0.486920045 | 0.00091274 |
| 11 | 11 | 0 | NLK | Q9UBE8 | wp117_DB0646 | DB0646 | down | −0.50096303 | 0.001896535 |
| 11 | 11 | 0 | NLK | Q9UBE8 | wp131_SB1.G.192.1 | SB1.G.192.1 | down | −0.563446834 | 0.00283154 |
| 11 | 11 | 0 | NLK | Q9UBE8 | wp137_DD.03.106 | DD.03.106 | down | −0.440995898 | 0.00605402 |
| 11 | 11 | 0 | NLK | Q9UBE8 | wp137_TL12.186 | TL12.186 | down | −0.515412597 | 0.00296344 |
| 11 | 11 | 0 | NLK | Q9UBE8 | wp160_DB0646_8 hr | DB0646 | down | −0.556942973 | 0.003804102 |
| 11 | 11 | 0 | NLK | Q9UBE8 | wp168_DB1113 | DB1113 | down | −0.855615819 | 3.52E−05 |
| 11 | 11 | 0 | RPS6KA3 | P51812 | wp076_MFH.5.116.1 | MFH.5.116.1 | down | −0.369014842 | 0.000752342 |
| 11 | 11 | 0 | RPS6KA3 | P51812 | wp076_MFH.5.103.1 | MFH.5.103.1 | down | −1.165448949 | 5.12E−06 |
| 11 | 11 | 0 | RPS6KA3 | P51812 | wp146_TMX.02.138 | TMX.02.138 | down | −0.402274783 | 0.003050057 |
| 11 | 11 | 0 | RPS6KA3 | P51812 | wp146_TMX.02.172 | TMX.02.172 | down | −0.834855023 | 0.000227929 |
| 10 | 10 | 0 | RPS6KA3 | P51812 | wp146_TMX.02.174 | TMX.02.174 | down | −0.431232146 | 0.002396687 |
| 10 | 10 | 0 | RPS6KA3 | P51812 | wp160_DB0646_8 hr | DB0646 | down | −0.333154046 | 3.53E−05 |
| 10 | 10 | 0 | RPS6KA3 | P51812 | wp168_DB0662 | DB0662 | down | −0.451979709 | 1.57E−06 |
| 10 | 10 | 0 | RPS6KA3 | P51812 | wp168_DB1113 | DB1113 | down | −0.50788897 | 2.24E−06 |
| 10 | 10 | 0 | STK10 | O94804 | wp016_TL12.186 | TL12.186 | down | −0.381931934 | 0.000453266 |
| 10 | 10 | 0 | STK10 | O94804 | wp016_LT2.49 | LT2.49 | down | −0.333626261 | 0.000796695 |
| 10 | 10 | 0 | STK10 | O94804 | wp016_SK.3.89 | SK.3.89 | down | −0.353933503 | 0.000623197 |
| 10 | 10 | 0 | STK10 | O94804 | wp056_WH.10417.099 | WH.10417.099 | down | −0.492343596 | 6.21E−05 |
| 10 | 10 | 0 | STK10 | O94804 | wp103_SB1.G.187 | SB1.G.187 | down | −0.337438718 | 0.000275333 |
| 10 | 10 | 0 | STK10 | O94804 | wp103_WH.10417.099 | WH.10417.099 | down | −0.475065193 | 5.95E−05 |
| 10 | 10 | 0 | STK10 | O94804 | wp107_WH.10417.099 | WH.10417.099 | down | −0.566116278 | 6.39E−07 |
| 10 | 10 | 0 | STK10 | O94804 | wp137_TL13.97 | TL13.97 | down | −0.347737137 | 3.66E−05 |
| 10 | 10 | 0 | TEC | P42680 | wp047_DD.03.119 | DD.03.119 | down | −2.271839509 | 3.21E−06 |
| 10 | 10 | 0 | TEC | P42680 | wp103_SK.3.91 | SK.3.91 | down | −0.649675507 | 0.002240563 |
| 10 | 10 | 0 | TEC | P42680 | wp107_SK.3.91 | SK.3.91 | down | −1.145690473 | 0.00034376 |
| 10 | 10 | 0 | TEC | P42680 | wp107_DB0646 | DB0646 | down | −0.82820896 | 0.000685451 |
| 10 | 10 | 0 | TEC | P42680 | wp134_DB118430 | DB118430 | down | −0.566631013 | 0.001673902 |
| 10 | 10 | 0 | TEC | P42680 | wp137_TL12.186 | TL12.186 | down | −2.110326588 | 9.03E−05 |
| 10 | 10 | 0 | TEC | P42680 | wp137_TL13.97 | TL13.97 | down | −1.711558198 | 0.000272432 |
| 10 | 10 | 0 | TEC | P42680 | wp146_TMX.02.174 | TMX.02.174 | down | −0.936152994 | 0.005488999 |
| 10 | 10 | 0 | YES1 | P07947 | wp105_DB0646 | DB0646 | down | −0.395730728 | 0.009334446 |
| 10 | 10 | 0 | YES1 | P07947 | wp131_TL12.186 | TL12.186 | down | −0.543732369 | 0.002267287 |
| 10 | 10 | 0 | YES1 | P07947 | wp131_SB1.G.192.1 | SB1.G.192.1 | down | −1.243814532 | 6.52E−05 |
| 10 | 10 | 0 | YES1 | P07947 | wp133_SK.3.91_4 hr | SK.3.91 | down | −0.502541377 | 0.00396364 |
| 10 | 10 | 0 | YES1 | P07947 | wp168_DB0661 | DB0661 | down | −0.522918919 | 0.001846002 |
| 10 | 10 | 0 | YES1 | P07947 | wp168_DB0662 | DB0662 | down | −0.802458585 | 0.000200066 |
| 10 | 10 | 0 | YES1 | P07947 | wp172_DB0646 | DB0646 | down | −0.470984336 | 0.00015388 |
| 10 | 10 | 0 | YES1 | P07947 | wp172_SB1.G.187 | SB1.G.187 | down | −0.532446218 | 6.42E−05 |
| 10 | 10 | 0 | AKT2 | P31751 | wp104_INY.02.164 | INY.02.164 | down | −1.077583759 | 2.43E−07 |
| 10 | 10 | 0 | AKT2 | P31751 | wp105_SK.3.91 | SK.3.91 | down | −0.872213443 | 1.05E−07 |
| 10 | 5 | 5 | AKT2 | P31751 | wp107_SK.3.91 | SK.3.91 | down | −0.414103693 | 2.29E−05 |
| 10 | 5 | 5 | AKT2 | P31751 | wp118_INY.03.041.01 | INY.03.041.01 | down | −2.777378768 | 1.02E−08 |
| 10 | 5 | 5 | AKT2 | P31751 | wp135_INY.03.041_01_8 hr | INY.03.041_01 | down | −2.273843423 | 5.77E−10 |
| 10 | 5 | 5 | AKT2 | P31751 | wp135_INY.03.041.01_4 hr | INY.03.041.01 | down | −1.738945032 | 8.00E−10 |
| 10 | 5 | 5 | AKT2 | P31751 | wp137_INY.02.164 | INY.02.164 | down | −3.155944869 | 2.26E−10 |
| 10 | 10 | 0 | BCKDK | O14874 | wp084_BSJ.04.132 | BSJ.04.132 | down | −0.518842453 | 0.000985769 |
| 10 | 10 | 0 | BCKDK | O14874 | wp084_BSJ.03.123 | BSJ.03.123 | down | −0.455295963 | 0.003776792 |
| 10 | 10 | 0 | BCKDK | O14874 | wp137_DD.03.106 | DD.03.106 | down | −0.327541257 | 0.006576697 |
| 10 | 10 | 0 | BCKDK | O14874 | wp137_INY.02.164 | INY.02.164 | down | −0.360000048 | 0.00429856 |
| 10 | 10 | 0 | BCKDK | O14874 | wp147_DD.03.106_4 uM | DD.03.106 | down | −0.583102951 | 0.009621684 |
| 10 | 10 | 0 | BCKDK | O14874 | wp147_DD.03.107_4 uM | DD.03.107 | down | −1.112855395 | 0.0005271 |
| 10 | 10 | 0 | BCKDK | O14874 | wp160_SK.3.91_1 hr | SK.3.91 | down | −0.868341934 | 0.006518736 |
| 10 | 10 | 0 | MAP4K5 | Q9Y4K4 | wp045_DB0646 | DB0646 | down | −0.410858914 | 0.002022897 |
| 10 | 10 | 0 | MAP4K5 | Q9Y4K4 | wp105_SB1.G.187 | SB1.G.187 | down | −0.348768151 | 6.71E−06 |
| 10 | 10 | 0 | MAP4K5 | Q9Y4K4 | wp107_SB1.G.187 | SB1.G.187 | down | −0.441149839 | 1.38E−05 |
| 9 | 9 | 0 | MAP4K5 | Q9Y4K4 | wp107_DB0646 | DB0646 | down | −0.334484814 | 8.66E−05 |
| 9 | 9 | 0 | MAP4K5 | Q9Y4K4 | wp152_DB1114 | DB1114 | down | −0.856736336 | 3.22E−06 |
| 9 | 9 | 0 | MAP4K5 | Q9Y4K4 | wp160_DB0646_8 hr | DB0646 | down | −0.500903106 | 0.000133356 |
| 9 | 9 | 0 | MAP4K5 | Q9Y4K4 | wp168_DB1113 | DB1113 | down | −1.754256579 | 1.57E−08 |
| 9 | 9 | 0 | PTK2B | Q14289-2 | wp107_SK.3.91 | SK.3.91 | down | −3.126393715 | 4.84E−08 |
| 9 | 9 | 0 | PTK2B | Q14289-2 | wp107_DB0646 | DB0646 | down | −2.484218435 | 3.50E−09 |
| 9 | 9 | 0 | PTK2B | Q14289-2 | wp117_DB0614 | DB0614 | down | −1.202514347 | 8.20E−06 |
| 9 | 9 | 0 | PTK2B | Q14289-2 | wp117_DB0646 | DB0646 | down | −2.583131778 | 3.99E−08 |
| 9 | 9 | 0 | PTK2B | Q14289-2 | wp168_DB0661 | DB0661 | down | −1.935495318 | 1.19E−09 |
| 9 | 9 | 0 | PTK2B | Q14289-2 | wp168_DB0662 | DB0662 | down | −3.244523022 | 1.79E−07 |
| 9 | 9 | 0 | PTK2B | Q14289-2 | wp168_DB1113 | DB1113 | down | −1.705194138 | 1.31E−09 |
| 9 | 9 | 0 | RPS6KA1 | Q15418-1 | wp107_SK.3.91 | SK.3.91 | down | −0.367148184 | 1.33E−05 |
| 9 | 9 | 0 | RPS6KA1 | Q15418-1 | wp131_TL12.186 | TL12.186 | down | −0.356211236 | 4.61E−06 |
| 9 | 9 | 0 | RPS6KA1 | Q15418-1 | wp160_DB0646_8 hr | DB0646 | down | −0.412977636 | 0.000208437 |
| 9 | 9 | 0 | RPS6KA1 | Q15418-1 | wp160_SK.3.91_4 hr | SK.3.91 | down | −0.362983106 | 0.000347624 |
| 9 | 9 | 0 | RPS6KA1 | Q15418-1 | wp168_DB0662 | DB0662 | down | −0.472415754 | 1.54E−05 |
| 9 | 9 | 0 | RPS6KA1 | Q15418-1 | wp168_DB1113 | DB1113 | down | −0.729723242 | 2.91E−07 |
| 9 | 9 | 0 | RPS6KA1 | Q15418-1 | wp172_SK.3.91 | SK.3.91 | down | −0.456109816 | 4.97E−05 |
| 9 | 8 | 1 | STK33 | Q9BYT3-1 | wp076_MFH.5.116.1 | MFH.5.116.1 | down | −1.381787254 | 0.002754088 |
| 9 | 8 | 1 | STK33 | Q9BYT3-1 | wp076_MFH.5.103.1 | MFH.5.103.1 | down | −2.784065751 | 0.000145167 |
| 9 | 8 | 1 | STK33 | Q9BYT3-1 | wp105_SK.3.91 | SK.3.91 | down | −2.057180782 | 2.58E−07 |
| 9 | 8 | 1 | STK33 | Q9BYT3-1 | wp146_TMX.02.138 | TMX.02.138 | down | −1.335417657 | 0.000289728 |
| 9 | 8 | 1 | STK33 | Q9BYT3-1 | wp146_TMX.02.172 | TMX.02.172 | down | −1.295180588 | 0.000323524 |
| 9 | 8 | 1 | STK33 | Q9BYT3-1 | wp146_TMX.02.174 | TMX.02.174 | down | −1.762557834 | 0.000106042 |
| 9 | 8 | 1 | STK33 | Q9BYT3-1 | wp172_SK.3.91 | SK.3.91 | down | −2.226973009 | 7.99E−05 |
| 9 | 8 | 1 | STK40 | Q8N2I9-4 | wp105_SK.3.91 | SK.3.91 | down | −0.605993802 | 0.003219194 |
| 9 | 1 | 8 | TNK2 | Q07912-3 | wp045_DB0663 | DB0663 | down | −0.927070942 | 0.008730978 |
| 8 | 8 | 0 | TNK2 | Q07912-3 | wp071_SB1.G.192.1 | SB1.G.192.1 | down | −1.727838542 | 0.003226077 |
| 8 | 8 | 0 | TNK2 | Q07912-3 | wp081_SB1.G.192.1 | SB1.G.192.1 | down | −0.902364454 | 0.000316675 |
| 8 | 8 | 0 | TNK2 | Q07912-3 | wp107_SK.3.91 | SK.3.91 | down | −1.17998954 | 0.001023444 |
| 8 | 8 | 0 | TNK2 | Q07912-3 | wp107_DB0646 | DB0646 | down | −0.825837876 | 0.005224286 |
| 8 | 8 | 0 | TNK2 | Q07912-3 | wp117_DB0646 | DB0646 | down | −0.983330485 | 1.69E−05 |
| 8 | 8 | 0 | TNK2 | Q07912-3 | wp149_ZNL.03.127 | ZNL.03.127 | down | −0.877764043 | 0.000392417 |
| 8 | 8 | 0 | AKT3 | Q9Y243-1 | wp104_INY.02.164 | INY.02.164 | down | −1.47841841 | 6.29E−07 |
| 8 | 8 | 0 | AKT3 | Q9Y243-1 | wp105_SK.3.91 | SK.3.91 | down | −0.778266185 | 8.85E−06 |
| 8 | 8 | 0 | AKT3 | Q9Y243-1 | wp118_INY.03.041.01 | INY.03.041.01 | down | −2.768698208 | 6.18E−07 |
| 8 | 8 | 0 | AKT3 | Q9Y243-1 | wp135_INY.03.041_01_8 hr | INY.03.041_01 | down | −2.215282966 | 1.04E−07 |
| 8 | 8 | 0 | AKT3 | Q9Y243-1 | wp135_INY.03.041.01_4 hr | INY.03.041.01 | down | −1.924681835 | 1.85E−07 |
| 8 | 8 | 0 | AKT3 | Q9Y243-1 | wp137_INY.02.164 | INY.02.164 | down | −3.14690657 | 1.34E−07 |
| 8 | 8 | 0 | FER | P16591-3 | wp114_DB0646 | DB0646 | down | −0.592127396 | 0.00021958 |
| 8 | 8 | 0 | FER | P16591-3 | wp114_SK.3.91 | SK.3.91 | down | −2.307708711 | 8.92E−07 |
| 8 | 8 | 0 | FER | P16591-3 | wp114_FMF.06.098.1 | FMF.06.098.1 | down | −0.398577122 | 0.001046767 |
| 8 | 8 | 0 | FER | P16591-3 | wp172_DB0646 | DB0646 | down | −1.014337598 | 5.34E−05 |
| 8 | 8 | 0 | FER | P16591-3 | wp172_SB1.G.187 | SB1.G.187 | down | −1.393631776 | 1.09E−05 |
| 8 | 8 | 0 | FER | P16591-3 | wp172_SK.3.91 | SK.3.91 | down | −2.816023319 | 1.09E−06 |
| 8 | 8 | 0 | FYN | P06241-1 | wp081_SB1.G.192.1 | SB1.G.192.1 | down | −0.931424974 | 0.001678815 |
| 8 | 8 | 0 | FYN | P06241-1 | wp105_SK.3.91 | SK.3.91 | down | −0.358631692 | 0.00348289 |
| 8 | 8 | 0 | FYN | P06241-1 | wp105_SB1.G.187 | SB1.G.187 | down | −0.769673436 | 0.000180865 |
| 8 | 8 | 0 | FYN | P06241-1 | wp105_DB0646 | DB0646 | down | −0.462140663 | 0.000635729 |
| 8 | 8 | 0 | FYN | P06241-1 | wp107_SK.3.91 | SK.3.91 | down | −0.428842703 | 0.000545606 |
| 8 | 8 | 0 | FYN | P06241-1 | wp107_SB1.G.187 | SB1.G.187 | down | −0.360064683 | 0.003093501 |
| 8 | 8 | 0 | IRAK1 | P51617 | wp056_SB1.G.187 | SB1.G.187 | down | −1.578479396 | 2.41E−05 |
| 8 | 8 | 0 | IRAK1 | P51617 | wp081_TL13.150 | TL13.150 | down | −2.834475854 | 1.36E−08 |
| 8 | 8 | 0 | IRAK1 | P51617 | wp103_SB1.G.187 | SB1.G.187 | down | −1.504293228 | 0.000116904 |
| 8 | 8 | 0 | IRAK1 | P51617 | wp105_SB1.G.187 | SB1.G.187 | down | −1.403879803 | 3.54E−06 |
| 8 | 8 | 0 | IRAK1 | P51617 | wp107_SB1.G.187 | SB1.G.187 | down | −1.995843679 | 2.94E−07 |
| 8 | 8 | 0 | IRAK1 | P51617 | wp172_SB1.G.187 | SB1.G.187 | down | −1.571747234 | 1.01E−05 |
| 8 | 8 | 0 | LIMK2 | P53671-3 | wp047_DD.03.106 | DD.03.106 | down | −0.961977779 | 3.11E−05 |
| 8 | 8 | 0 | LIMK2 | P53671-3 | wp105_SK.3.91 | SK.3.91 | down | −0.840097519 | 8.42E−06 |
| 8 | 8 | 0 | LIMK2 | P53671-3 | wp105_DB0646 | DB0646 | down | −0.729889831 | 6.59E−06 |
| 8 | 8 | 0 | LIMK2 | P53671-3 | wp147_DD.03.106_1 uM | DD.03.106 | down | −0.873645672 | 3.03E−06 |
| 8 | 8 | 0 | LIMK2 | P53671-3 | wp147_DD.03.106_2 uM | DD.03.106 | down | −1.081503108 | 1.01E−06 |
| 8 | 8 | 0 | LIMK2 | P53671-3 | wp147_DD.03.106_4 uM | DD.03.106 | down | −0.931929469 | 2.18E−06 |
| 8 | 8 | 0 | MAP3K7 | O43318 | wp056_SB1.G.187 | SB1.G.187 | down | −1.002897711 | 6.74E−06 |
| 8 | 8 | 0 | MAP3K7 | O43318 | wp103_SB1.G.187 | SB1.G.187 | down | −1.37390723 | 9.99E−07 |
| 8 | 8 | 0 | MAP3K7 | O43318 | wp105_SB1.G.187 | SB1.G.187 | down | −1.031433007 | 1.25E−07 |
| 8 | 8 | 0 | MAP3K7 | O43318 | wp107_SB1.G.187 | SB1.G.187 | down | −1.330238322 | 7.08E−08 |
| 8 | 8 | 0 | MAP3K7 | O43318 | wp152_DB1114 | DB1114 | down | −0.902639968 | 1.07E−07 |
| 8 | 8 | 0 | MAP3K7 | O43318 | wp172_SB1.G.187 | SB1.G.187 | down | −0.577245069 | 0.006198544 |
| 8 | 8 | 0 | MAPK7 | Q13164-1 | wp016_SK.3.91_1 uM | SK.3.91 | down | −0.392385718 | 0.007912806 |
| 8 | 8 | 0 | MAPK7 | Q13164-1 | wp076_MFH.5.103.1 | MFH.5.103.1 | down | −0.414223988 | 0.004198789 |
| 8 | 8 | 0 | MAPK7 | Q13164-1 | wp108_FMF.06.098.1 | FMF.06.098.1 | down | −0.434660583 | 0.000401871 |
| 8 | 8 | 0 | MAPK7 | Q13164-1 | wp131_FMF.06.098.1 | FMF.06.098.1 | down | −0.463718578 | 5.93E−06 |
| 8 | 8 | 0 | MAPK7 | Q13164-1 | wp137_TL12.186 | TL12.186 | down | −0.333419408 | 0.000196327 |
| 8 | 8 | 0 | MAPK7 | Q13164-1 | wp168_DB1113 | DB1113 | down | −0.766013029 | 1.76E−06 |
| 8 | 8 | 0 | MARK2 | Q7KZI7-1 | wp107_SK.3.91 | SK.3.91 | down | −0.52663474 | 0.000115655 |
| 8 | 8 | 0 | MARK2 | Q7KZI7-1 | wp117_RSS0680 | RSS0680 | down | −0.428583283 | 0.001497894 |
| 8 | 8 | 0 | MARK2 | Q7KZI7-1 | wp134_BSJ.04.178 | BSJ.04.178 | down | −0.968568944 | 4.76E−05 |
| 8 | 8 | 0 | MARK2 | Q7KZI7-1 | wp172_SK.3.91 | SK.3.91 | down | −0.635128221 | 4.23E−05 |
| 8 | 8 | 0 | MARK2 | Q7KZI7-1 | wp195_BSJ.05.026 | BSJ.05.026 | down | −1.238580981 | 1.39E−08 |
| 8 | 8 | 0 | NUAK1 | O60285 | wp017_WH.9533.153 | WH.9533.153 | down | −1.366779057 | 9.35E−05 |
| 8 | 8 | 0 | NUAK1 | O60285 | wp076_MFH.5.126.1 | MFH.5.126.1 | down | −1.649135288 | 2.06E−05 |
| 8 | 8 | 0 | NUAK1 | O60285 | wp076_MFH.5.116.1 | MFH.5.116.1 | down | −0.707792462 | 0.000790621 |
| 8 | 8 | 0 | NUAK1 | O60285 | wp076_MFH.5.115.2 | MFH.5.115.2 | down | −0.526351963 | 0.002677085 |
| 8 | 8 | 0 | NUAK1 | O60285 | wp146_TMX.02.138 | TMX.02.138 | down | −0.825413725 | 0.007238957 |
| 8 | 8 | 0 | NUAK1 | O60285 | wp146_TMX.02.174 | TMX.02.174 | down | −1.360705215 | 0.001293039 |
| 8 | 8 | 0 | SRC | P12931 | wp152_DB0646 | DB0646 | down | −0.394055991 | 0.000399921 |
| 8 | 8 | 0 | SRC | P12931 | wp160_DB0646_4 hr | DB0646 | down | −0.518561148 | 3.62E−05 |
| 8 | 8 | 0 | SRC | P12931 | wp160_DB0646_8 hr | DB0646 | down | −0.683284399 | 1.19E−05 |
| 8 | 8 | 0 | SRC | P12931 | wp160_SK.3.91_4 hr | SK.3.91 | down | −0.529179341 | 3.33E−05 |
| 8 | 8 | 0 | SRC | P12931 | wp168_DB0661 | DB0661 | down | −0.66849148 | 6.71E−06 |
| 8 | 8 | 0 | SRC | P12931 | wp168_DB0662 | DB0662 | down | −0.901795732 | 1.40E−06 |
| 8 | 8 | 0 | ABL2 | P42684-5 | wp047_DD.03.119 | DD.03.119 | down | −1.047217017 | 8.46E−06 |
| 8 | 8 | 0 | ABL2 | P42684-5 | wp131_FMF.06.098.1 | FMF.06.098.1 | down | −0.644264171 | 3.27E−05 |
| 8 | 8 | 0 | ABL2 | P42684-5 | wp131_TL12.186 | TL12.186 | down | −0.80230497 | 2.03E−05 |
| 8 | 8 | 0 | ABL2 | P42684-5 | wp131_SB1.G.192.1 | SB1.G.192.1 | down | −2.800086029 | 3.25E−07 |
| 8 | 8 | 0 | ABL2 | P42684-5 | wp172_SK.3.91 | SK.3.91 | down | −0.713860318 | 5.86E−05 |
| 8 | 8 | 0 | AKT1 | P31749 | wp104_INY.02.164 | INY.02.164 | down | −0.79259464 | 0.000107849 |
| 8 | 8 | 0 | AKT1 | P31749 | wp118_INY.03.041.01 | INY.03.041.01 | down | −2.25794728 | 4.85E−07 |
| 7 | 7 | 0 | AKT1 | P31749 | wp135_INY.03.041_01_8 hr | INY.03.041_01 | down | −2.272698292 | 1.39E−10 |
| 7 | 7 | 0 | AKT1 | P31749 | wp135_INY.03.041.01_4 hr | INY.03.041.01 | down | −1.224795943 | 9.02E−09 |
| 7 | 7 | 0 | AKT1 | P31749 | wp137_INY.02.164 | INY.02.164 | down | −3.205545882 | 2.64E−10 |
| 7 | 7 | 0 | BMPR1A | P36894 | wp105_SK.3.91 | SK.3.91 | down | −0.407701732 | 0.003687664 |
| 7 | 7 | 0 | BMPR1A | P36894 | wp107_SK.3.91 | SK.3.91 | down | −1.103106357 | 6.76E−06 |
| 7 | 7 | 0 | BMPR1A | P36894 | wp131_TL12.186 | TL12.186 | down | −0.833369609 | 8.10E−05 |
| 7 | 7 | 0 | BMPR1A | P36894 | wp133_SK.3.91_4 hr | SK.3.91 | down | −0.800664316 | 0.004155623 |
| 7 | 7 | 0 | BMPR1A | P36894 | wp137_TL12.186 | TL12.186 | down | −0.656576627 | 0.004246127 |
| 7 | 7 | 0 | EPHB6 | O15197-1 | wp071_SB1.G.192.1 | SB1.G.192.1 | down | −0.625904739 | 0.001238461 |
| 7 | 7 | 0 | EPHB6 | O15197-1 | wp131_SB1.G.192.1 | SB1.G.192.1 | down | −0.518900558 | 0.000110492 |
| 7 | 7 | 0 | EPHB6 | O15197-1 | wp160_DB0646_4 hr | DB0646 | down | −0.339585599 | 0.000793886 |
| 7 | 7 | 0 | EPHB6 | O15197-1 | wp160_DB0646_8 hr | DB0646 | down | −0.654766746 | 5.87E−05 |
| 7 | 7 | 0 | EPHB6 | O15197-1 | wp168_DB0662 | DB0662 | down | −0.735959361 | 0.004275826 |
| 7 | 7 | 0 | LRRK2 | Q5S007 | wp160_DB0646_1 hr | DB0646 | down | −1.165044295 | 0.000152554 |
| 7 | 7 | 0 | LRRK2 | Q5S007 | wp160_DB0646_2 hr | DB0646 | down | −1.180656608 | 0.000144666 |
| 7 | 7 | 0 | LRRK2 | Q5S007 | wp160_DB0646_4 hr | DB0646 | down | −1.306991269 | 9.64E−05 |
| 7 | 7 | 0 | LRRK2 | Q5S007 | wp160_DB0646_8 hr | DB0646 | down | −1.747073046 | 3.00E−05 |
| 7 | 7 | 0 | LRRK2 | Q5S007 | wp160_SK.3.91_1 hr | SK.3.91 | down | −0.844713722 | 0.000544815 |
| 7 | 7 | 0 | RPS6KB1 | P23443-1 | wp080_RSS0680 | RSS0680 | down | −0.435757867 | 0.00099775 |
| 7 | 7 | 0 | RPS6KB1 | P23443-1 | wp107_SK.3.91 | SK.3.91 | down | −0.41970503 | 0.00231545 |
| 7 | 7 | 0 | RPS6KB1 | P23443-1 | wp117_RSS0680 | RSS0680 | down | −0.38927093 | 0.000206736 |
| 7 | 7 | 0 | RPS6KB1 | P23443-1 | wp195_BSJ.05.026 | BSJ.05.026 | down | −0.562884481 | 3.24E−05 |
| 7 | 7 | 0 | RPS6KB1 | P23443-1 | wp195_JWG.123 | JWG.123 | down | −0.34257461 | 0.000450829 |
| 7 | 7 | 0 | SNRK | Q9NRH2 | wp080_RSS0680 | RSS0680 | down | −0.556662347 | 0.001236305 |
| 7 | 7 | 0 | SNRK | Q9NRH2 | wp117_RSS0680 | RSS0680 | down | −0.632132264 | 1.40E−05 |
| 7 | 7 | 0 | SNRK | Q9NRH2 | wp134_BSJ.04.178 | BSJ.04.178 | down | −1.52301866 | 4.60E−06 |
| 7 | 7 | 0 | SNRK | Q9NRH2 | wp195_BSJ.05.026 | BSJ.05.026 | down | −1.856363982 | 5.37E−08 |
| 7 | 7 | 0 | SNRK | Q9NRH2 | wp195_JWG.123 | JWG.123 | down | −1.197496011 | 6.19E−07 |
| 7 | 7 | 0 | STK4 | Q13043-1 | wp017_WH.9533.153 | WH.9533.153 | down | −0.493567671 | 0.000306667 |
| 7 | 7 | 0 | STK4 | Q13043-1 | wp056_WH.10417.099 | WH.10417.099 | down | −0.715702368 | 7.55E−05 |
| 7 | 7 | 0 | STK4 | Q13043-1 | wp103_WH.10417.099 | WH.10417.099 | down | −0.738740573 | 2.04E−06 |
| 7 | 7 | 0 | STK4 | Q13043-1 | wp107_WH.10417.099 | WH.10417.099 | down | −0.718765128 | 2.13E−05 |
| 7 | 7 | 0 | STK4 | Q13043-1 | wp172_WH.10417.099 | WH.10417.099 | down | −0.598304593 | 0.003151374 |
| 7 | 7 | 0 | ABL1 | P00519 | wp152_DB1114 | DB1114 | down | −0.621267296 | 3.21E−07 |
| 7 | 7 | 0 | ABL1 | P00519 | wp160_SK.3.91_4 hr | SK.3.91 | down | −0.354459134 | 0.003530976 |
| 6 | 6 | 0 | ABL1 | P00519 | wp168_DB0662 | DB0662 | down | −0.761672515 | 2.66E−07 |
| 6 | 6 | 0 | ABL1 | P00519 | wp168_DB1113 | DB1113 | down | −1.014277493 | 7.45E−08 |
| 6 | 6 | 0 | AURKB | Q96GD4 | wp160_SK.3.91_4 hr | SK.3.91 | down | −0.498367651 | 0.000293648 |
| 6 | 6 | 0 | AURKB | Q96GD4 | wp164_SB1.G.194 | SB1.G.194 | down | −0.488293586 | 0.000152971 |
| 6 | 6 | 0 | AURKB | Q96GD4 | wp172_SK.3.91 | SK.3.91 | down | −1.109927086 | 1.05E−06 |
| 6 | 6 | 0 | AURKB | Q96GD4 | wp195_BJG.04.203 | BJG.04.203 | down | −0.506385834 | 5.34E−05 |
| 6 | 6 | 0 | CDK10 | Q15131-1 | wp076_MFH.5.103.1 | MFH.5.103.1 | down | −0.698823929 | 0.008064503 |
| 6 | 6 | 0 | CDK10 | Q15131-1 | wp131_TL12.186 | TL12.186 | down | −0.935047944 | 2.50E−05 |
| 6 | 6 | 0 | CDK10 | Q15131-1 | wp133_SK.3.91_4 hr | SK.3.91 | down | −0.544017595 | 0.001567632 |
| 6 | 6 | 0 | CDK10 | Q15131-1 | wp134_BSJ.04.178 | BSJ.04.178 | down | −0.654649527 | 0.000983126 |
| 6 | 6 | 0 | CDK14 | O94921-1 | wp017_WH.9533.153 | WH.9533.153 | down | −0.463684985 | 0.001166413 |
| 6 | 6 | 0 | CDK14 | O94921-1 | wp076_MFH.5.126.1 | MFH.5.126.1 | down | −0.341346376 | 0.004899609 |
| 6 | 6 | 0 | CDK14 | O94921-1 | wp172_SB1.G.187 | SB1.G.187 | down | −1.524912734 | 0.000233028 |
| 6 | 6 | 0 | CDK14 | O94921-1 | wp172_SK.3.91 | SK.3.91 | down | −0.817553859 | 0.000708349 |
| 6 | 6 | 0 | CDK9 | P50750 | wp160_SK.3.91_2 hr | SK.3.91 | down | −0.403978714 | 0.000964795 |
| 6 | 6 | 0 | CDK9 | P50750 | wp160_SK.3.91_4 hr | SK.3.91 | down | −1.067854135 | 2.02E−05 |
| 6 | 6 | 0 | CDK9 | P50750 | wp164_SB1.G.194 | SB1.G.194 | down | −0.839140953 | 5.66E−06 |
| 6 | 6 | 0 | CDK9 | P50750 | wp172_SK.3.91 | SK.3.91 | down | −1.423462978 | 1.91E−06 |
| 6 | 6 | 0 | IRAK4 | Q9NWZ3-1 | wp108_FMF.06.098.1 | FMF.06.098.1 | down | −0.650492698 | 3.34E−05 |
| 6 | 6 | 0 | IRAK4 | Q9NWZ3-1 | wp131_FMF.06.098.1 | FMF.06.098.1 | down | −0.708586993 | 1.42E−05 |
| 6 | 6 | 0 | IRAK4 | Q9NWZ3-1 | wp131_TL12.186 | TL12.186 | down | −0.429255255 | 1.04E−05 |
| 6 | 6 | 0 | IRAK4 | Q9NWZ3-1 | wp137_TL12.186 | TL12.186 | down | −0.418518213 | 5.57E−06 |
| 6 | 6 | 0 | MAPK8 | P45983-2 | wp103_DB0646 | DB0646 | down | −0.610874817 | 4.72E−05 |
| 6 | 6 | 0 | MAPK8 | P45983-2 | wp107_DB0646 | DB0646 | down | −0.723017007 | 1.57E−06 |
| 5 | 5 | 0 | MAPK8 | P45983-2 | wp117_DB0614 | DB0614 | down | −0.632830736 | 5.89E−06 |
| 5 | 5 | 0 | MAPK8 | P45983-2 | wp117_DB0646 | DB0646 | down | −0.783458474 | 1.13E−06 |
| 5 | 5 | 0 | PDK3 | Q15120-1 | wp160_SK.3.91_1 hr | SK.3.91 | down | −0.980147823 | 0.006529529 |
| 5 | 5 | 0 | PRKAA2 | P54646 | wp105_SK.3.91 | SK.3.91 | down | −0.902881212 | 2.90E−06 |
| 5 | 5 | 0 | PRKAA2 | P54646 | wp146_TMX.02.138 | TMX.02.138 | down | −1.475675213 | 0.00029302 |
| 5 | 5 | 0 | PRKAA2 | P54646 | wp146_TMX.02.174 | TMX.02.174 | down | −1.826067237 | 0.000135543 |
| 5 | 5 | 0 | PRKAA2 | P54646 | wp172_SK.3.91 | SK.3.91 | down | −0.92603174 | 1.15E−06 |
| 5 | 5 | 0 | RIPK2 | O43353-1 | wp105_SB1.G.187 | SB1.G.187 | down | −0.572016518 | 0.002942934 |
| 5 | 5 | 0 | RIPK2 | O43353-1 | wp105_DB0646 | DB0646 | down | −0.42154218 | 0.009947713 |
| 5 | 5 | 0 | RIPK2 | O43353-1 | wp114_SB1.G.187 | SB1.G.187 | down | −0.882799975 | 0.000737868 |
| 5 | 5 | 0 | RIPK2 | O43353-1 | wp172_DB0646 | DB0646 | down | −0.477939872 | 0.006511762 |
| 5 | 5 | 0 | BTK | Q06187-2 | wp055_DD.03.171 | DD.03.171 | down | −3.077185183 | 3.29E−10 |
| 5 | 5 | 0 | BTK | Q06187-2 | wp055_DD.04.118 | DD.04.118 | down | −3.263337177 | 1.28E−09 |
| 5 | 5 | 0 | CDC7 | O00311-1 | wp108_FMF.06.098.1 | FMF.06.098.1 | down | −0.332297303 | 0.003823993 |
| 5 | 5 | 0 | CDC7 | O00311-1 | wp131_FMF.06.098.1 | FMF.06.098.1 | down | −0.410949298 | 0.00120241 |
| 5 | 5 | 0 | CDC7 | O00311-1 | wp133_SK.3.91_4 hr | SK.3.91 | down | −0.3651703 | 0.00064216 |
| 5 | 5 | 0 | CDK12 | Q9NYV4-2 | wp107_SK.3.91 | SK.3.91 | down | −1.042042951 | 3.48E−08 |
| 5 | 5 | 0 | CDK12 | Q9NYV4-2 | wp160_SK.3.91_2 hr | SK.3.91 | down | −0.45578445 | 1.69E−05 |
| 5 | 5 | 0 | CDK12 | Q9NYV4-2 | wp160_SK.3.91_4 hr | SK.3.91 | down | −0.844759645 | 1.39E−06 |
| 5 | 5 | 0 | CDK17 | Q00537-2 | wp137_DD.03.106 | DD.03.106 | down | −0.868159486 | 6.86E−06 |
| 5 | 5 | 0 | CDK17 | Q00537-2 | wp137_TL12.186 | TL12.186 | down | −0.702633411 | 2.18E−05 |
| 5 | 5 | 0 | CDK17 | Q00537-2 | wp137_TL13.97 | TL13.97 | down | −0.568293467 | 6.85E−05 |
| 5 | 5 | 0 | CHEK1 | O14757 | wp076_MFH.5.126.1 | MFH.5.126.1 | down | −0.36063559 | 0.00353339 |
| 5 | 5 | 0 | CHEK1 | O14757 | wp108_FMF.06.098.1 | FMF.06.098.1 | down | −0.367439972 | 0.000314772 |
| 5 | 5 | 0 | CHEK1 | O14757 | wp114_FMF.06.098.1 | FMF.06.098.1 | down | −0.412421467 | 0.001214217 |
| 5 | 5 | 0 | CLK1 | P49759 | wp168_DB0661 | DB0661 | down | −0.331866205 | 0.002260027 |
| 5 | 5 | 0 | CLK1 | P49759 | wp195_ZZ.01.31B | ZZ.01.31B | down | −0.348247461 | 0.000432935 |
| 5 | 5 | 0 | CSNK1A1 | P48729-2 | wp076_MFH.5.126.1 | MFH.5.126.1 | down | −0.458637095 | 0.000418096 |
| 5 | 5 | 0 | CSNK1A1 | P48729-2 | wp076_MFH.5.115.2 | MFH.5.115.2 | down | −0.348985098 | 0.001317143 |
| 5 | 5 | 0 | CSNK1A1 | P48729-2 | wp107_WH.10417.099 | WH.10417.099 | down | −2.428013905 | 1.20E−09 |
| 5 | 5 | 0 | CSNK1D | P48730-1 | wp017_WH.9533.153 | WH.9533.153 | down | −0.778834345 | 2.63E−05 |
| 5 | 5 | 0 | CSNK1D | P48730-1 | wp131_TL12.186 | TL12.186 | down | −0.355089737 | 0.00424498 |
| 5 | 5 | 0 | CSNK1D | P48730-1 | wp133_SK.3.91_4 hr | SK.3.91 | down | −0.458525843 | 0.000203698 |
| 5 | 5 | 0 | EIF2AK2 | P19525 | wp117_DB0614 | DB0614 | down | −0.322089522 | 0.000121915 |
| 5 | 5 | 0 | EIF2AK2 | P19525 | wp147_DD.03.106_2 uM | DD.03.106 | down | −0.354501556 | 1.06E−05 |
| 5 | 5 | 0 | EIF2AK2 | P19525 | wp147_DD.03.106_4 uM | DD.03.106 | down | −0.40423566 | 5.43E−06 |
| 5 | 5 | 0 | EPHB2 | P29323 | wp107_SK.3.91 | SK.3.91 | down | −0.629301042 | 0.00115023 |
| 5 | 5 | 0 | EPHB2 | P29323 | wp195_BJG.04.203 | BJG.04.203 | down | −0.453541034 | 0.001476879 |
| 5 | 5 | 0 | EPHB2 | P29323 | wp195_JWG.148 | JWG.148 | down | −0.359588697 | 0.004444413 |
| 5 | 5 | 0 | LYN | P07948-2 | wp105_SB1.G.187 | SB1.G.187 | down | −1.087146089 | 3.70E−07 |
| 4 | 4 | 0 | LYN | P07948-2 | wp105_DB0646 | DB0646 | down | −0.9849778 | 3.91E−06 |
| 4 | 4 | 0 | LYN | P07948-2 | wp114_SB1.G.187 | SB1.G.187 | down | −0.440752037 | 0.000343684 |
| 4 | 4 | 0 | MAP2K5 | Q13163-1 | wp071_SB1.G.181 | SB1.G.181 | down | −1.137828406 | 0.00013055 |
| 4 | 4 | 0 | MAP2K5 | Q13163-1 | wp071_SB1.G.200 | SB1.G.200 | down | −1.433260113 | 4.33E−05 |
| 4 | 4 | 0 | MAP2K5 | Q13163-1 | wp074_dRAF.3 | dRAF.3 | down | −0.5935186 | 0.006444941 |
| 4 | 4 | 0 | MAPK8 | P45983-3 | wp105_DB0646 | DB0646 | down | −0.40939353 | 0.001683019 |
| 4 | 4 | 0 | MAPK8 | P45983-3 | wp160_DB0646_4 hr | DB0646 | down | −0.610956063 | 1.34E−05 |
| 4 | 4 | 0 | MAPK8 | P45983-3 | wp160_DB0646_8 hr | DB0646 | down | −1.010159186 | 1.76E−06 |
| 4 | 4 | 0 | MARK2 | Q7KZI7-14 | wp105_SK.3.91 | SK.3.91 | down | −0.415861618 | 0.004169892 |
| 4 | 4 | 0 | MARK2 | Q7KZI7-14 | wp131_TL12.186 | TL12.186 | down | −0.48675544 | 1.63E−06 |
| 4 | 4 | 0 | MARK2 | Q7KZI7-14 | wp133_SK.3.91_4 hr | SK.3.91 | down | −0.408608783 | 3.08E−06 |
| 4 | 4 | 0 | PDIK1L | Q8N165 | wp152_DB1114 | DB1114 | down | −0.482010744 | 0.001386938 |
| 4 | 4 | 0 | PDIK1L | Q8N165 | wp168_DB0662 | DB0662 | down | −0.327159383 | 0.000191836 |
| 4 | 4 | 0 | PDIK1L | Q8N165 | wp168_DB1113 | DB1113 | down | −0.533438327 | 1.70E−05 |
| 4 | 4 | 0 | PIM2 | Q9P1W9 | wp017_WH.9533.153 | WH.9533.153 | down | −0.493588132 | 0.000581663 |
| 4 | 4 | 0 | PIM2 | Q9P1W9 | wp076_MFH.5.126.1 | MFH.5.126.1 | down | −3.019183352 | 1.85E−05 |
| 4 | 4 | 0 | PIM2 | Q9P1W9 | wp076_MFH.5.115.2 | MFH.5.115.2 | down | −1.679133576 | 0.000237251 |
| 4 | 4 | 0 | PRKAA1 | Q13131 | wp160_SK.3.91_2 hr | SK.3.91 | down | −0.328417912 | 0.000231315 |
| 4 | 4 | 0 | PRKAA1 | Q13131 | wp160_SK.3.91_4 hr | SK.3.91 | down | −0.611706985 | 1.91E−05 |
| 4 | 4 | 0 | PRKAA1 | Q13131 | wp172_SK.3.91 | SK.3.91 | down | −0.454197166 | 4.72E−05 |
| 3 | 2 | 1 | RPSGKA4 | O75676 | wp108_FMF.06.098.1 | FMF.06.098.1 | down | −0.419633813 | 0.000227637 |
| 3 | 2 | 1 | RPS6KA4 | O75676 | wp131_FMF.06.098.1 | FMF.06.098.1 | down | −0.535130375 | 2.78E−05 |
| 3 | 3 | 0 | RPS6KA4 | O75676 | wp146_TMX.02.174 | TMX.02.174 | down | −1.500289043 | 0.004117443 |
| 3 | 3 | 0 | SBK1 | Q52WVX2 | wp107_SK.3.91 | SK.3.91 | down | −0.921037788 | 0.001449833 |
| 3 | 3 | 0 | SBK1 | Q52WX2 | wp133_SK.3.91_2 hr | SK.3.91 | down | −0.685244724 | 0.0003185 |
| 3 | 3 | 0 | SBK1 | Q52WX2 | wp133_SK.3.91_4 hr | SK.3.91 | down | −0.902921654 | 7.62E−05 |
| 3 | 3 | 0 | SIK3 | Q9Y2K2 | wp168_DB1113 | DB1113 | down | −1.602158067 | 2.66E−08 |
| 3 | 3 | 0 | SIK3 | Q9Y2K2 | wp195_BSJ.05.026 | BSJ.05.026 | down | −1.186279383 | 6.15E−07 |
| 3 | 3 | 0 | SIK3 | Q9Y2K2 | wp195_JWG.123 | JWG.123 | down | −1.322198842 | 3.36E−07 |
| 3 | 3 | 0 | STK38 | Q15208 | wp137_TL13.97 | TL13.97 | down | −0.39116144 | 5.99E−05 |
| 3 | 3 | 0 | STK38 | Q15208 | wp146_TMX.02.138 | TMX.02.138 | down | −0.347595468 | 0.002054209 |
| 3 | 3 | 0 | STK38 | Q15208 | wp146_TMX.02.174 | TMX.02.174 | down | −0.411710376 | 0.001131916 |
| 3 | 3 | 0 | CAMKK1 | Q8N5S9-2 | wp045_RSS0628 | RSS0628 | down | −0.341192329 | 0.004291185 |
| 3 | 3 | 0 | CAMKK1 | Q8N5S9-2 | wp045_DB0614 | DB0614 | down | −0.648660736 | 0.000304942 |
| 3 | 3 | 0 | CDK11A | Q9UQ88-1 | wp068_FMF.04.147.1 | FMF.04.147.1 | down | −0.738608386 | 0.003511913 |
| 3 | 3 | 0 | CDK11A | Q9UQ88-1 | wp105_DB0646 | DB0646 | down | −0.868003768 | 0.001302571 |
| 3 | 3 | 0 | CDK11B | P21127 | wp152_DB0646 | DB0646 | down | −0.594754445 | 2.67E−08 |
| 3 | 3 | 0 | CDK11B | P21127 | wp152_DB1114 | DB1114 | down | −0.959292424 | 1.89E−09 |
| 3 | 3 | 0 | CDK16 | Q00536-1 | wp172_SK.3.91 | SK.3.91 | down | −0.81688154 | 9.31E−05 |
| 3 | 3 | 0 | CDK16 | Q00536-1 | wp195_JWG.123 | JWG.123 | down | −0.502598502 | 0.001772193 |
| 3 | 3 | 0 | CDK18 | Q07002-3 | wp105_SK.3.91 | SK.3.91 | down | −1.23289594 | 0.000160131 |
| 3 | 3 | 0 | CDK18 | Q07002-3 | wp114_SK.3.91 | SK.3.91 | down | −0.829450347 | 0.000595555 |
| 3 | 3 | 0 | COQ8B | Q96D53-1 | wp137_DD.03.106 | DD.03.106 | down | −0.34423313 | 0.00527359 |
| 3 | 3 | 0 | COQ8B | Q96D53-1 | wp137_TL12.186 | TL12.186 | down | −0.493366118 | 0.000947042 |
| 3 | 3 | 0 | EPHA1 | P21709 | wp114_SB1.G.187 | SB1.G.187 | down | −0.355157954 | 0.001091621 |
| 3 | 3 | 0 | EPHA1 | P21709 | wp114_FMF.06.098.1 | FMF.06.098.1 | down | −0.422026514 | 0.000555752 |
| 3 | 3 | 0 | EPHA2 | P29317 | wp114_SB1.G.187 | SB1.G.187 | down | −0.401042478 | 0.002550441 |
| 3 | 3 | 0 | EPHA2 | P29317 | wp172_SB1.G.187 | SB1.G.187 | down | −0.410711829 | 0.009095498 |
| 3 | 3 | 0 | ERN1 | O75460 | wp076_MFH.5.126.1 | MFH.5.126.1 | down | −0.48816302 | 0.002222446 |
| 3 | 3 | 0 | FER | P16591 | wp152_DB0646 | DB0646 | down | −1.698451471 | 6.90E−08 |
| 3 | 3 | 0 | FER | P16591 | wp152_DB1114 | DB1114 | down | −1.165199495 | 5.51E−07 |
| 3 | 3 | 0 | GSK3B | P49841-1 | wp172_WH.10417.099 | WH.10417.099 | down | −0.82979411 | 1.22E−05 |
| 3 | 3 | 0 | GSK3B | P49841-1 | wp195_BJG.04.203 | BJG.04.203 | down | −0.534845602 | 6.74E−06 |
| 3 | 3 | 0 | IRAK3 | Q9Y616 | wp146_TMX.02.138 | TMX.02.138 | down | −1.543631119 | 0.000464051 |
| 3 | 3 | 0 | IRAK3 | Q9Y616 | wp146_TMX.02.174 | TMX.02.174 | down | −1.343399546 | 0.000763035 |
| 3 | 3 | 0 | LCK | P06239 | wp152_DB0646 | DB0646 | down | −0.446823671 | 1.73E−06 |
| 3 | 3 | 0 | LCK | P06239 | wp152_DB1114 | DB1114 | down | −0.557129124 | 5.14E−07 |
| 3 | 3 | 0 | LYN | P07948-1 | wp172_DB0646 | DB0646 | down | −0.793809631 | 0.000420271 |
| 3 | 3 | 0 | LYN | P07948-1 | wp172_SB1.G.187 | SB1.G.187 | down | −1.229749001 | 1.90E−05 |
| 3 | 3 | 0 | MAP3K21 | Q5TCX8 | wp131_TL12.186 | TL12.186 | down | −0.969041075 | 9.27E−05 |
| 3 | 3 | 0 | MAP3K21 | Q5TCX8 | wp133_SK.3.91_4 hr | SK.3.91 | down | −0.46848396 | 0.007675733 |
| 2 | 2 | 0 | MAPK10 | P53779 | wp104_TMX.01.160.1_0.25 | TMX.01.160.1 | down | −0.574306624 | 0.000707359 |
| 2 | 2 | 0 | MAPK10 | P53779 | wp104_TMX.01.160.1_1 | TMX.01.160.1 | down | −0.574337328 | 0.00070718 |
| 2 | 2 | 0 | MAPK11 | Q15759 | wp105_DB0646 | DB0646 | down | −0.399798619 | 0.007759989 |
| 2 | 2 | 0 | MAPK11 | Q15759 | wp114_DB0646 | DB0646 | down | −0.620876831 | 0.003164096 |
| 2 | 2 | 0 | MAPK12 | P53778 | wp105_DB0646 | DB0646 | down | −0.593873693 | 5.41E−05 |
| 2 | 2 | 0 | MAPK12 | P53778 | wp172_DB0646 | DB0646 | down | −1.4571321 | 0.000873625 |
| 2 | 2 | 0 | MAPK9 | P45984 | wp152_DB0646 | DB0646 | down | −2.160783676 | 1.65E−08 |
| 2 | 2 | 0 | MAPK9 | P45984 | wp152_DB1114 | DB1114 | down | −1.181053348 | 4.63E−07 |
| 2 | 2 | 0 | MAPKAPK3 | Q16644 | wp152_DB1114 | DB1114 | down | −1.951663126 | 2.64E−08 |
| 2 | 2 | 0 | MAPKAPK3 | Q16644 | wp168_DB1113 | DB1113 | down | −3.127075579 | 3.11E−09 |
| 2 | 1 | 1 | MARK3 | P27448-5 | wp134_BSJ.04.178 | BSJ.04.178 | down | −0.49772035 | 1.84E−05 |
| 2 | 2 | 0 | MARK3 | P27448-5 | wp195_BSJ.05.026 | BSJ.05.026 | down | −0.824233509 | 7.10E−07 |
| 2 | 2 | 0 | MAST3 | O60307 | wp081_TL13.178 | TL13.178 | down | −0.485227015 | 0.003588563 |
| 2 | 2 | 0 | MAST3 | O60307 | wp107_SK.3.91 | SK.3.91 | down | −0.498369043 | 0.001449192 |
| 2 | 2 | 0 | PIK3CG | P48736 | wp068_FMF.04.147.1 | FMF.04.147.1 | down | −0.35813172 | 0.000137797 |
| 2 | 2 | 0 | PIK3CG | P48736 | wp076_MFH.5.126.1 | MFH.5.126.1 | down | −0.37353765 | 0.000959182 |
| 2 | 2 | 0 | PTK2B | Q14289 | wp152_DB0646 | DB0646 | down | −2.997834184 | 2.42E−10 |
| 2 | 2 | 0 | PTK2B | Q14289 | wp152_DB1114 | DB1114 | down | −2.038540346 | 2.05E−09 |
| 2 | 2 | 0 | PTK6 | Q13882 | wp114_SK.3.91 | SK.3.91 | down | −0.417080119 | 0.001473103 |
| 2 | 2 | 0 | PTK6 | Q13882 | wp114_FMF.06.098.1 | FMF.06.098.1 | down | −0.440148546 | 0.001196357 |
| 2 | 2 | 0 | RPS6KC1 | Q96S38 | wp105_SK.3.91 | SK.3.91 | down | −0.332674694 | 0.000209053 |
| 2 | 2 | 0 | RPS6KC1 | Q96S38 | wp107_SK.3.91 | SK.3.91 | down | −0.362293236 | 0.002156696 |
| 2 | 2 | 0 | SGK3 | Q96BR1-1 | wp068_FMF.04.147.1 | FMF.04.147.1 | down | −0.338923305 | 0.001418121 |
| 2 | 2 | 0 | TAOK3 | Q9H2K8 | wp103_SB1.G.187 | SB1.G.187 | down | −0.372651305 | 0.00075821 |
| 2 | 2 | 0 | TAOK3 | Q9H2K8 | wp107_SB1.G.187 | SB1.G.187 | down | −0.385775236 | 0.000339263 |
| 2 | 2 | 0 | CAMKK1 | Q8N5S9 | wp117_DB0614 | DB0614 | down | −0.470986973 | 0.000566904 |
| 2 | 2 | 0 | CDK11B | P21127-3 | wp103_DB0646 | DB0646 | down | −0.333351198 | 0.00943752 |
| 2 | 2 | 0 | CIT | O14578 | wp152_DB1114 | DB1114 | down | −0.389961279 | 1.22E−06 |
| 2 | 2 | 0 | DAPK1 | P53355-3 | wp076_MFH.5.126.1 | MFH.5.126.1 | down | −0.448764353 | 0.008986695 |
| 2 | 2 | 0 | DDR2 | Q16832 | wp105_DB0646 | DB0646 | down | −0.474073961 | 0.000296564 |
| 2 | 2 | 0 | EEF2K | O00418 | wp137_TL12.186 | TL12.186 | down | −0.34432662 | 0.000260038 |
| 2 | 2 | 0 | EIF2AK4 | Q9P2K8-1 | wp080_RSS0680 | RSS0680 | down | −0.331138848 | 0.000118812 |
| 2 | 2 | 0 | EPHB3 | P54753 | wp114_DB0646 | DB0646 | down | −0.417454794 | 0.001719906 |
| 2 | 1 | 1 | EPHB4 | P54760 | wp172_SK.3.91 | SK.3.91 | down | −0.505852369 | 0.003258308 |
| 2 | 2 | 0 | FGFR1 | P11362-21 | wp108_FMF.06.098.1 | FMF.06.098.1 | down | −0.412055939 | 0.005370099 |
| 2 | 2 | 0 | FGFR2 | P21802-11 | wp088_SB1.G.194 | SB1.G.194 | down | −0.480406987 | 0.00803761 |
| 2 | 2 | 0 | FLT1 | P17948-1 | wp131_TL12.186 | TL12.186 | down | −0.69898705 | 0.001113287 |
| 2 | 2 | 0 | FYN | P06241 | wp152_DB0646 | DB0646 | down | −0.368491318 | 2.80E−05 |
| 1 | 1 | 0 | HASPIN | Q8TF76-1 | wp137_TL12.186 | TL12.186 | down | −0.368048245 | 0.000545675 |
| 1 | 1 | 0 | HIPK1 | Q86Z02 | wp133_SK.3.91_4 hr | SK.3.91 | down | −0.400929476 | 0.000939958 |
| 1 | 1 | 0 | MAP3K12 | Q12852-2 | wp105_SK.3.91 | SK.3.91 | down | −0.693120681 | 0.000115403 |
| 1 | 1 | 0 | MAPK7 | Q13164 | wp152_DB1114 | DB1114 | down | −0.661678323 | 1.45E−06 |
| 1 | 1 | 0 | MAPK8 | P45983-1 | wp172_DB0646 | DB0646 | down | −0.376012028 | 0.000731898 |
| 1 | 1 | 0 | MAPK8 | P45983 | wp152_DB0646 | DB0646 | down | −0.84329995 | 5.91E−07 |
| 1 | 1 | 0 | MAPKAPK5 | Q8IW41 | wp080_RSS0680 | RSS0680 | down | −0.335534707 | 0.003094372 |
| 1 | 1 | 0 | MELK | Q14680 | wp131_FMF.06.098.1 | FMF.06.098.1 | down | −0.379990618 | 6.94E−05 |
| 1 | 1 | 0 | MINK1 | Q8N4C8-1 | wp107_WH.10417.099 | WH.10417.099 | down | −0.566053217 | 0.002786944 |
| 1 | 1 | 0 | MKNK2 | Q9HBH9 | wp152_DB0646 | DB0646 | down | −0.444693372 | 0.005847652 |
| 1 | 1 | 0 | NEK1 | Q96PY6-3 | wp146_TMX.02.174 | TMX.02.174 | down | −0.387083977 | 0.006478632 |
| 1 | 1 | 0 | NEK3 | P51956 | wp146_TMX.02.174 | TMX.02.174 | down | −0.849696633 | 0.001264062 |
| 1 | 1 | 0 | NEK4 | P51957 | wp146_TMX.02.174 | TMX.02.174 | down | −0.950846271 | 0.000535482 |
| 1 | 1 | 0 | PKMYT1 | Q99640 | wp131_SB1.G.192.1 | SB1.G.192.1 | down | −0.730092326 | 0.000770154 |
| 1 | 1 | 0 | PKN3 | Q6P5Z2 | wp114_FMF.06.098.1 | FMF.06.098.1 | down | −0.451022075 | 0.00783628 |
| 1 | 1 | 0 | PRKCI | P41743 | wp056_ZNL.02.096 | ZNL.02.096 | down | −0.53649759 | 0.000706844 |
| 1 | 1 | 0 | PTK2 | Q05397-5 | wp012_TL13.112 | TL13.112 | down | −0.621717636 | 4.81E−07 |
| 1 | 1 | 0 | RPS6KA1 | Q15418 | wp152_DB1114 | DB1114 | down | −0.396240898 | 2.44E−06 |
| 1 | 1 | 0 | RPS6KA6 | Q9UK32 | wp105_SK.3.91 | SK.3.91 | down | −0.830042207 | 0.000835969 |
| 1 | 1 | 0 | STK32C | Q86UX6 | wp114_SK.3.91 | SK.3.91 | down | −0.618895408 | 0.004738851 |
| 1 | 1 | 0 | TAOK2 | Q9UL54-2 | wp160_DB0646_8 hr | DB0646 | down | −0.597276145 | 6.49E−05 |
| 1 | 1 | 0 | TAOK2 | Q9UL54 | wp152_DB1114 | DB1114 | down | −0.323995768 | 0.000844712 |
| 1 | 1 | 0 | TBCK | Q8TEA7 | wp056_ZNL.02.096 | ZNL.02.096 | down | −0.333435587 | 0.005044453 |
| 1 | 1 | 0 | TBK1 | Q9UHD2 | wp146_TMX.02.174 | TMX.02.174 | down | −0.595093068 | 0.000790975 |
| 1 | 1 | 0 | TESK2 | Q96S53-1 | wp147_DD.03.106_4 uM | DD.03.106 | down | −0.383064428 | 0.008054565 |
| 1 | 1 | 0 | TGFBR1 | P36897-2 | wp107_SK.3.91 | SK.3.91 | down | −0.526388154 | 0.001917002 |
| 1 | 1 | 0 | TNK1 | Q13470-2 | wp114_SK.3.91 | SK.3.91 | down | −0.739352912 | 0.00014822 |
| 1 | 1 | 0 | TNK2 | Q07912 | wp152_DB1114 | DB1114 | down | −0.59780861 | 0.002328073 |
| 1 | 1 | 0 | TRIB3 | Q96RU7 | wp114_SK.3.91 | SK.3.91 | down | −1.211063558 | 0.007544404 |
| 1 | 1 | 0 | TRPM7 | Q96QT4 | wp107_SK.3.91 | SK.3.91 | down | −0.324102636 | 0.002522147 |
| 1 | 1 | 0 | TYK2 | P29597 | wp195_JWG.148 | JWG.148 | down | −0.518651417 | 0.000133891 |
| 1 | 1 | 0 | ULK3 | Q6PHR2-4 | wp107_SK.3.91 | SK.3.91 | down | −1.084344254 | 5.82E−06 |

What is claimed is:

1. A bifunctional compound for targeted degradation of at least one kinase, which is represented by any one of structures: 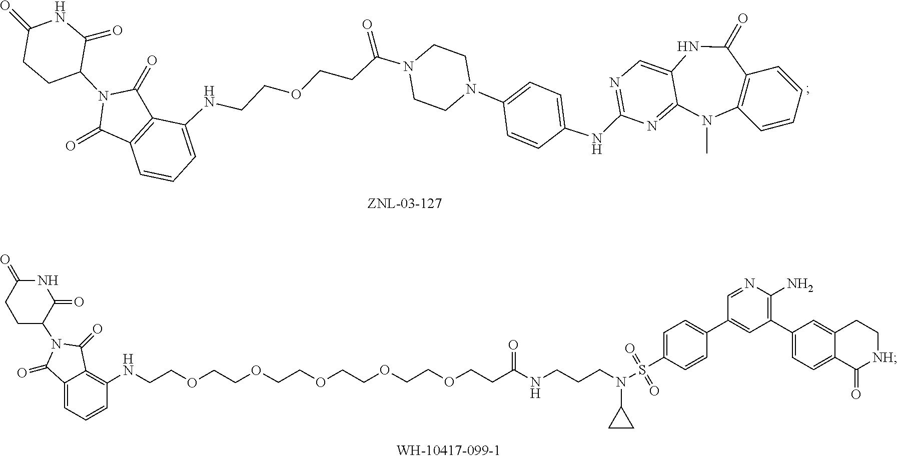 [SMILES: CN1c2ccccc2C(=O)Nc2cnc(Nc3ccc(N4CCN(C(=O)CCOCCNc5cccc6c5C(=O)N(C5CCC(=O)NC5=O)C6=O)CC4)cc3)nc21 ; Nc1ncc(-c2ccc(S(=O)(=O)N(CCCNC(=O)CCOCCOCCOCCOCCOCCNc3cccc4c3C(=O)N(C3CCC(=O)NC3=O)C4=O)C3CC3)cc2)cc1-c1ccc2c(c1)CCNC2=O] 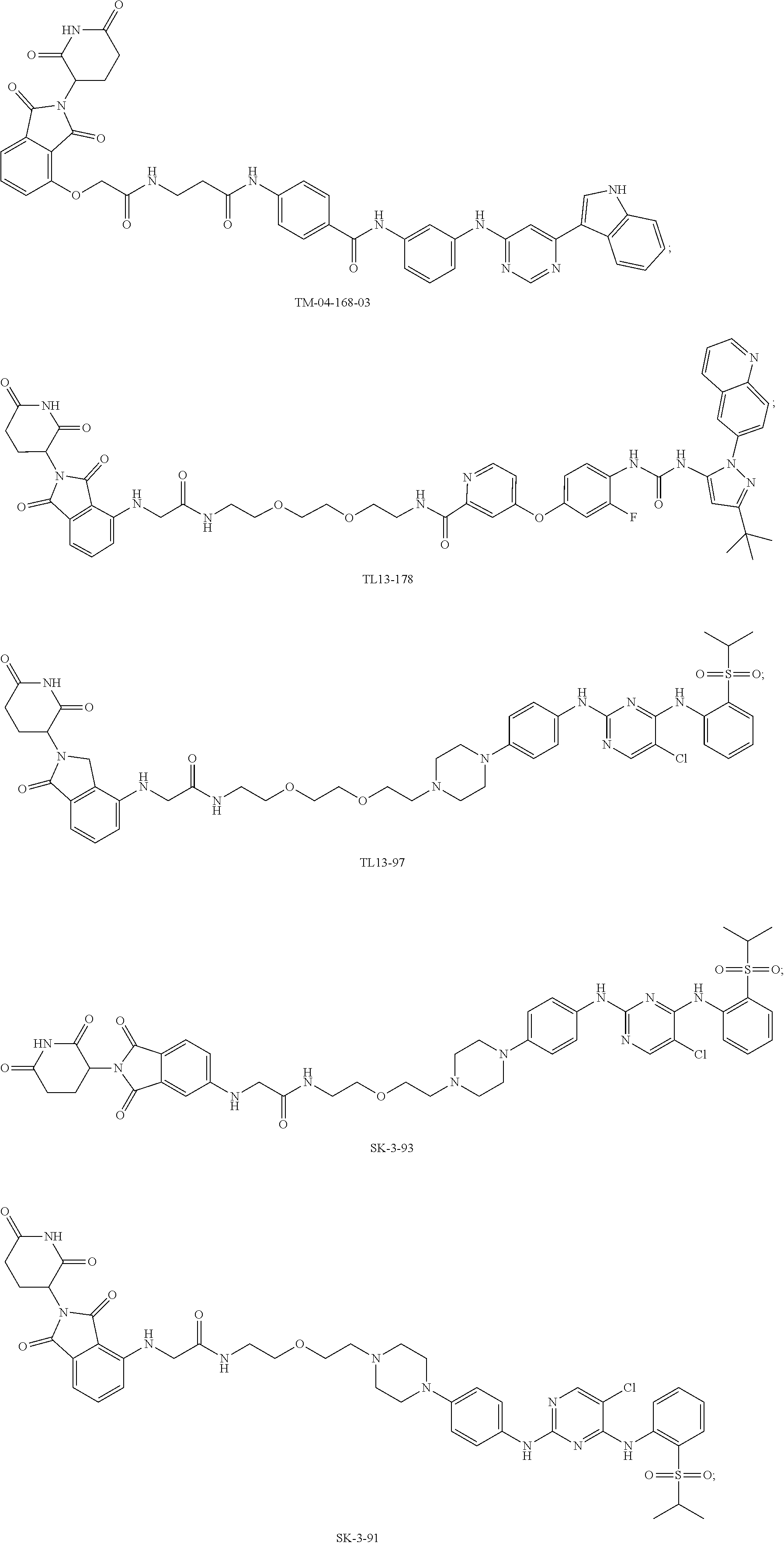 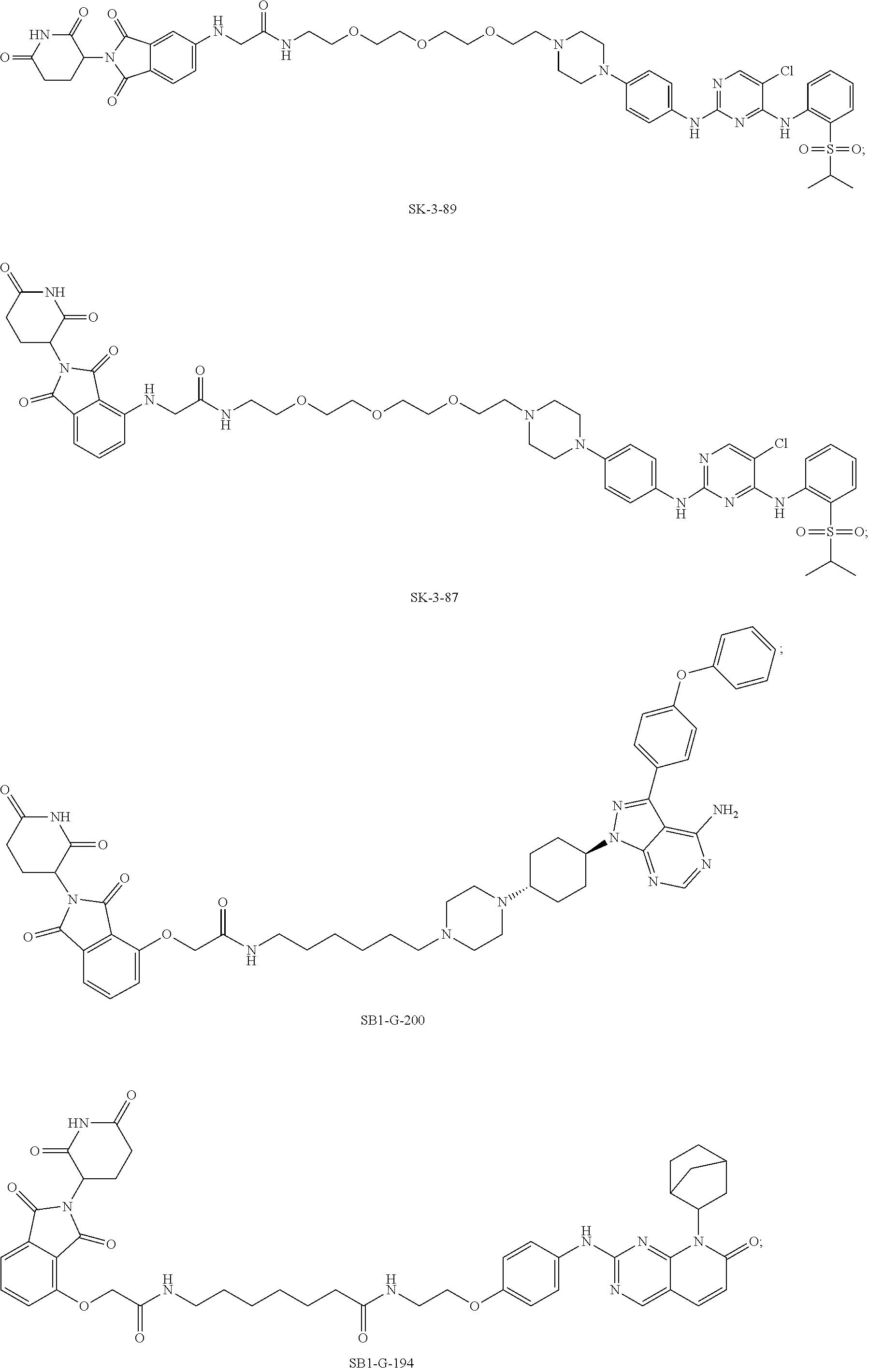 [SMILES: CC(C)S(=O)(=O)c1ccccc1Nc1nc(Nc2ccc(N3CCN(CCOCCOCCOCCNC(=O)CNc4ccc5c(c4)C(=O)N(C4CCC(=O)NC4=O)C5=O)CC3)cc2)ncc1Cl ; CC(C)S(=O)(=O)c1ccccc1Nc1nc(Nc2ccc(N3CCN(CCOCCOCCOCCNC(=O)CNc4cccc5c4C(=O)N(C4CCC(=O)NC4=O)C5=O)CC3)cc2)ncc1Cl ; Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1 ; O=C(CCCCCCNC(=O)COc1cccc2c1C(=O)N(C1CCC(=O)NC1=O)C2=O)NCCOc1ccc(Nc2ncc3ccc(=O)n(C4CC5CCC4C5)c3n2)cc1] 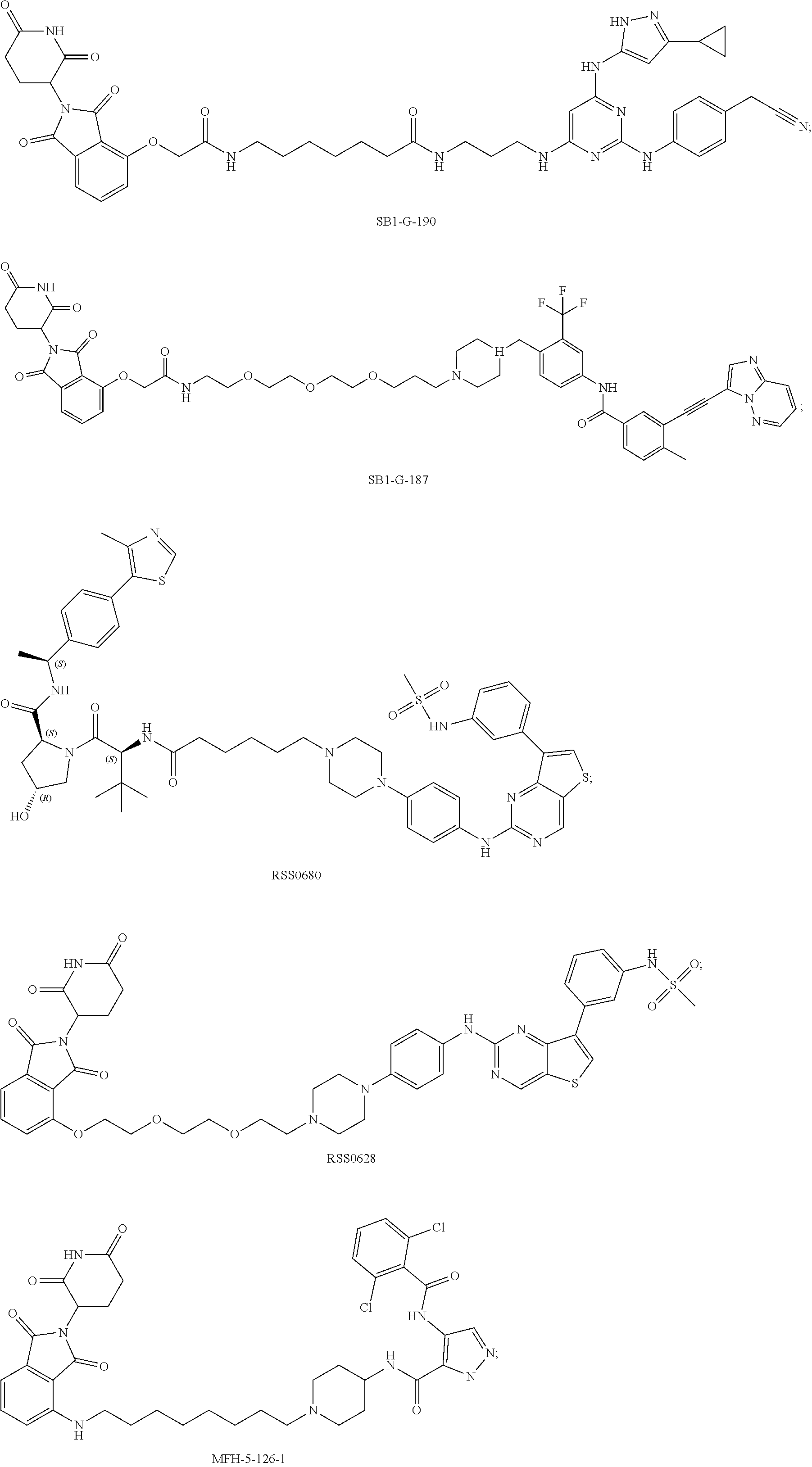 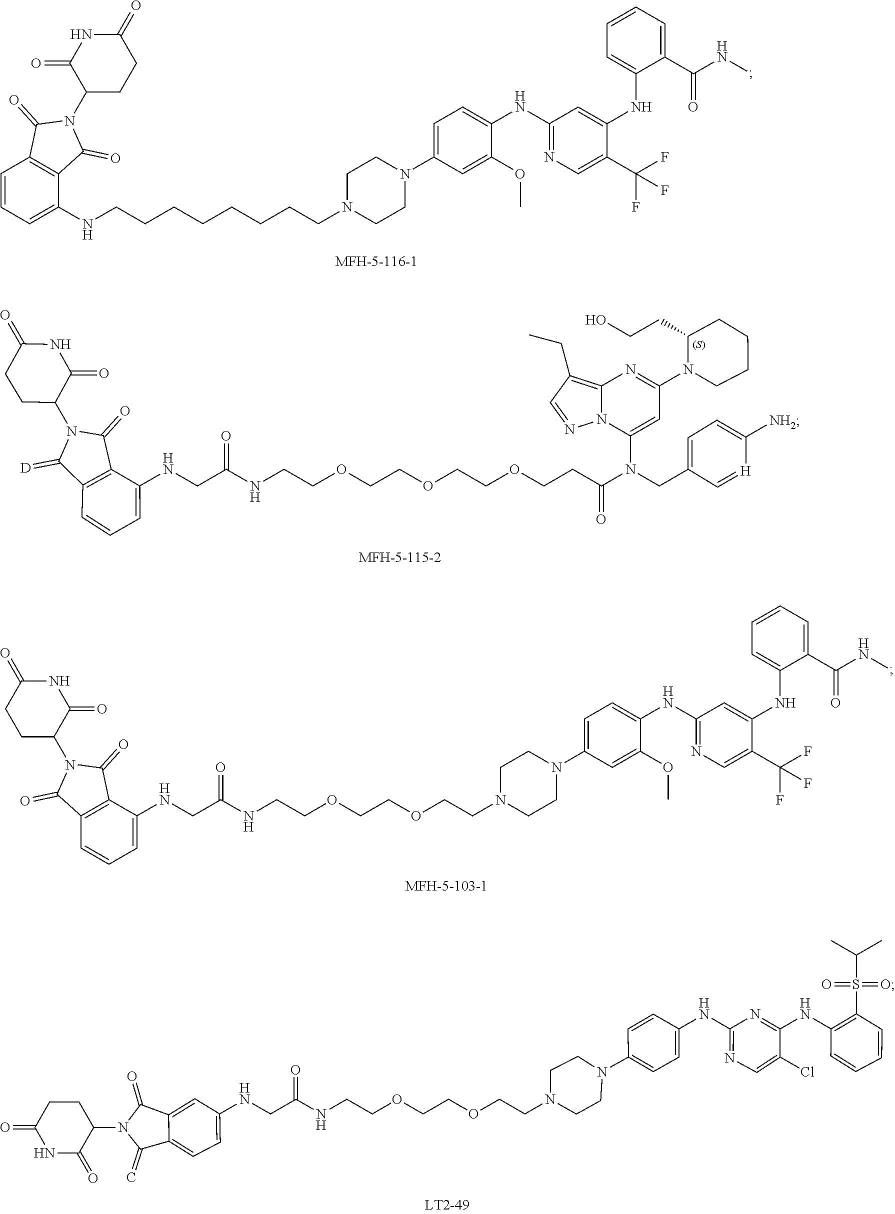 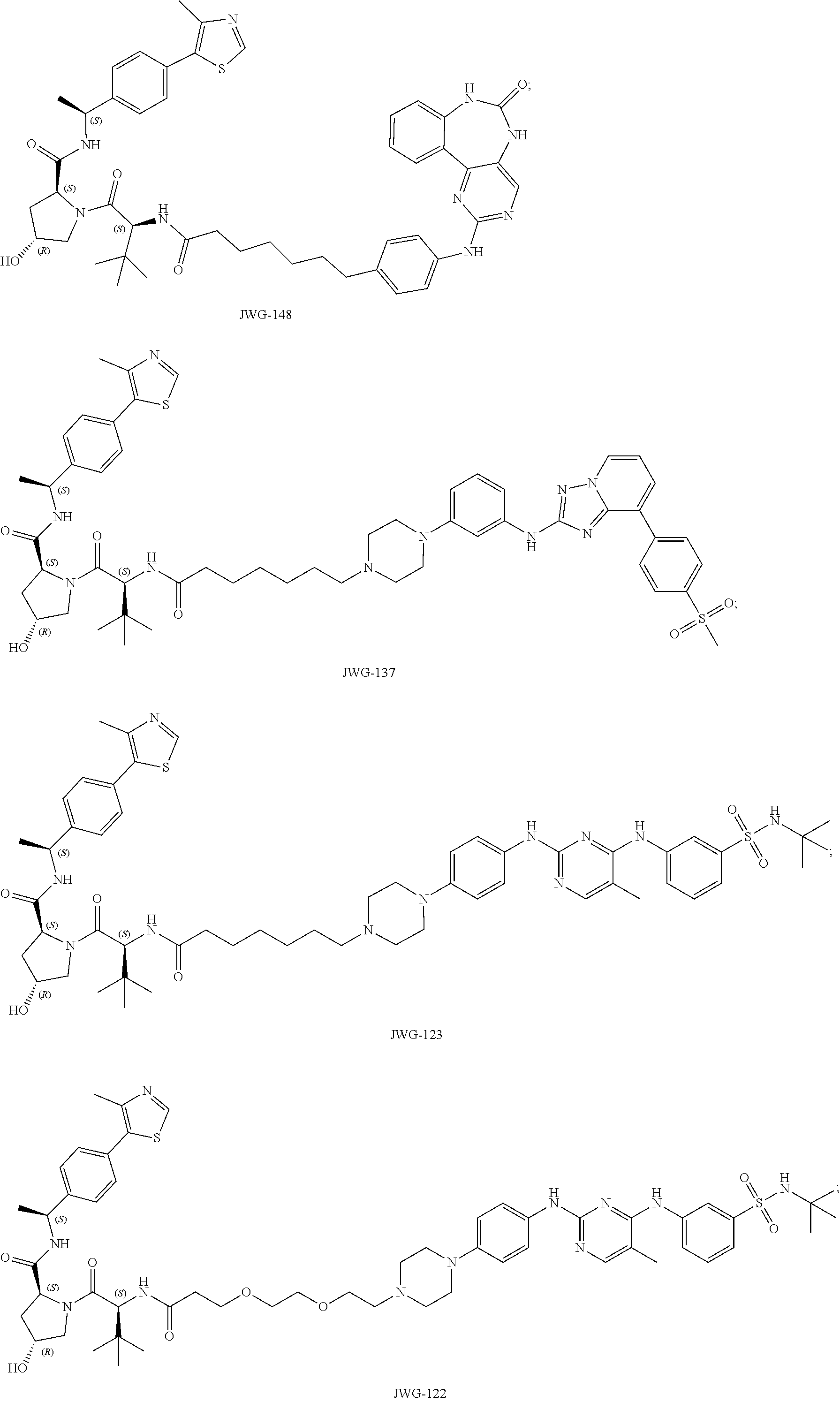 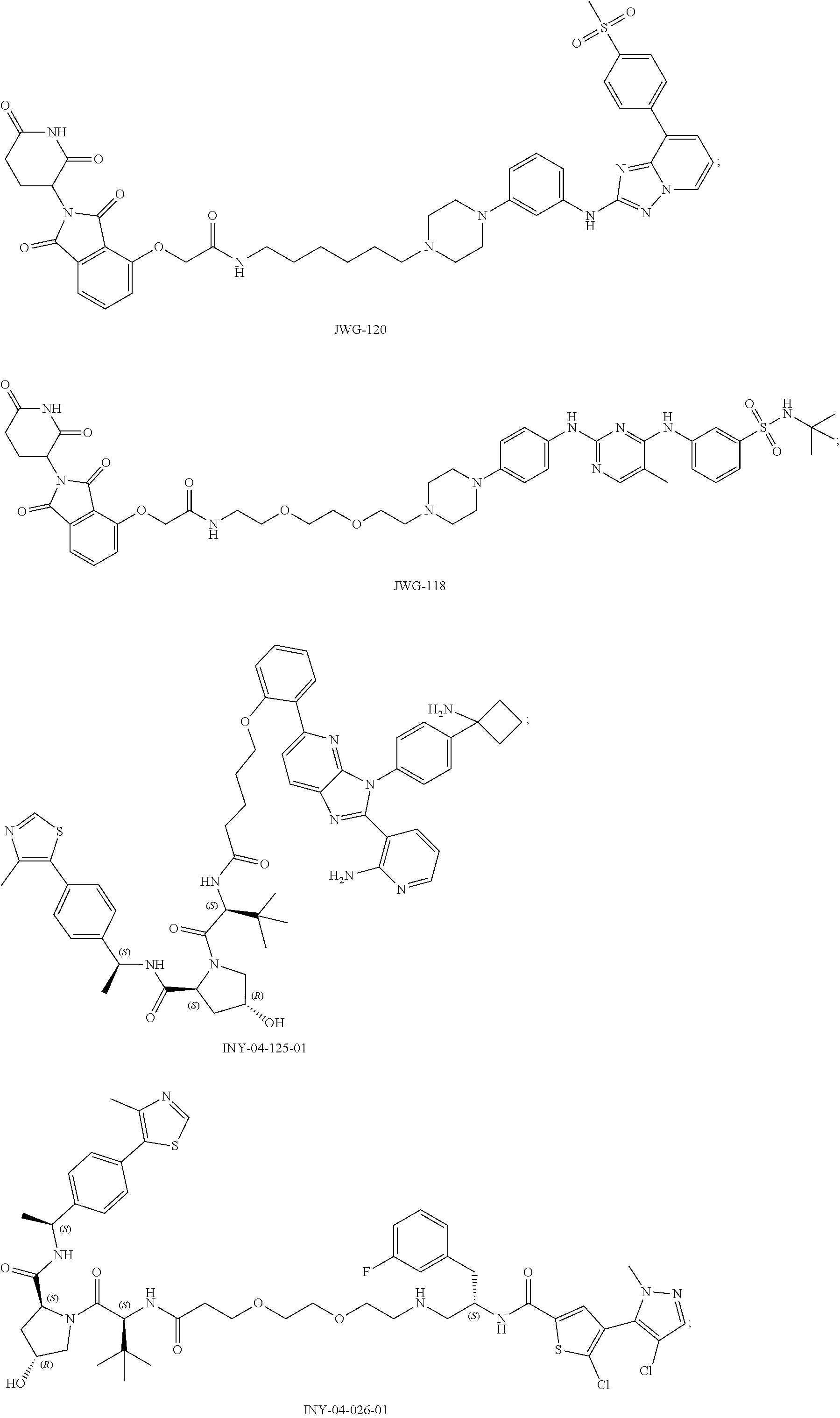 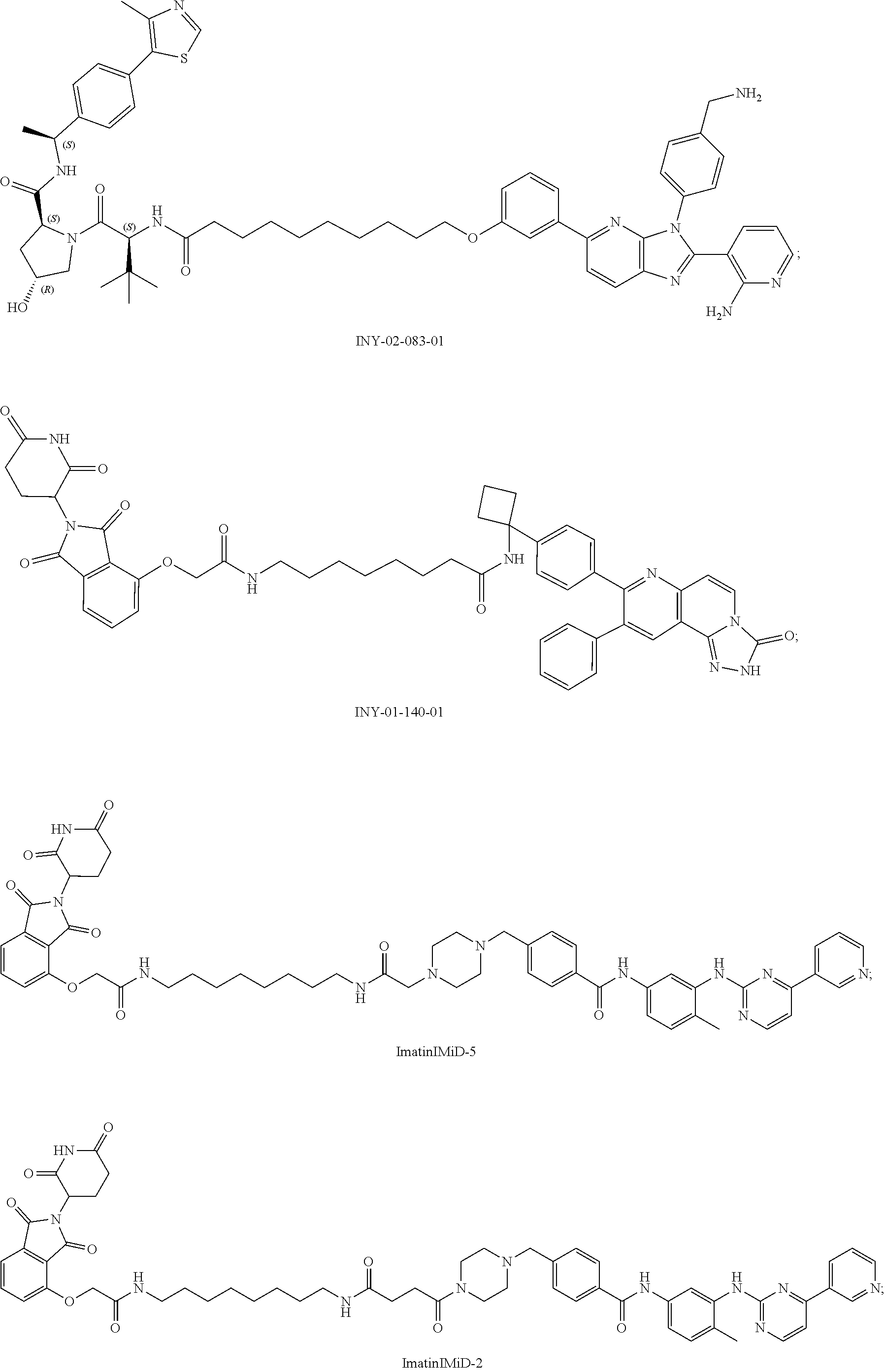 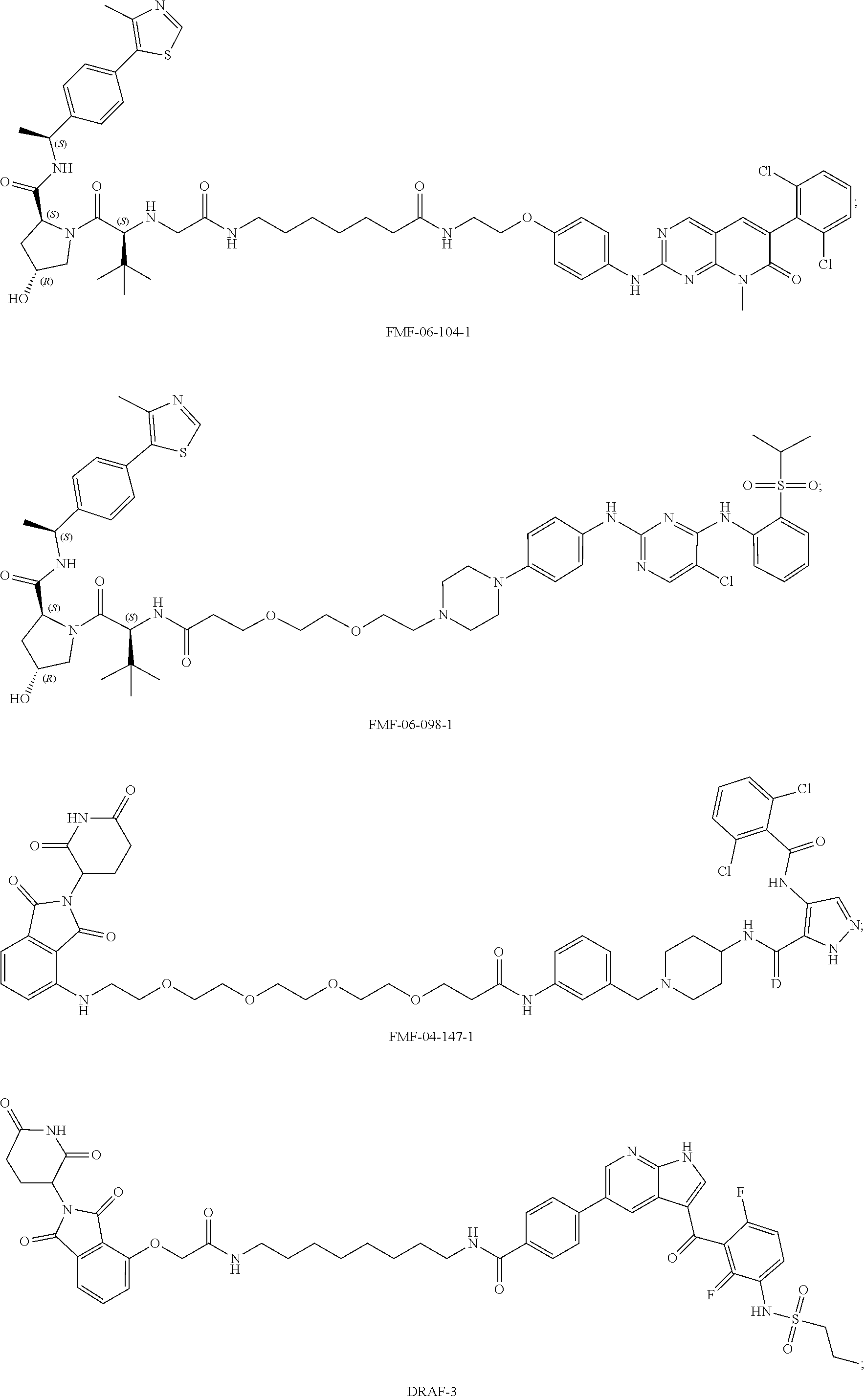 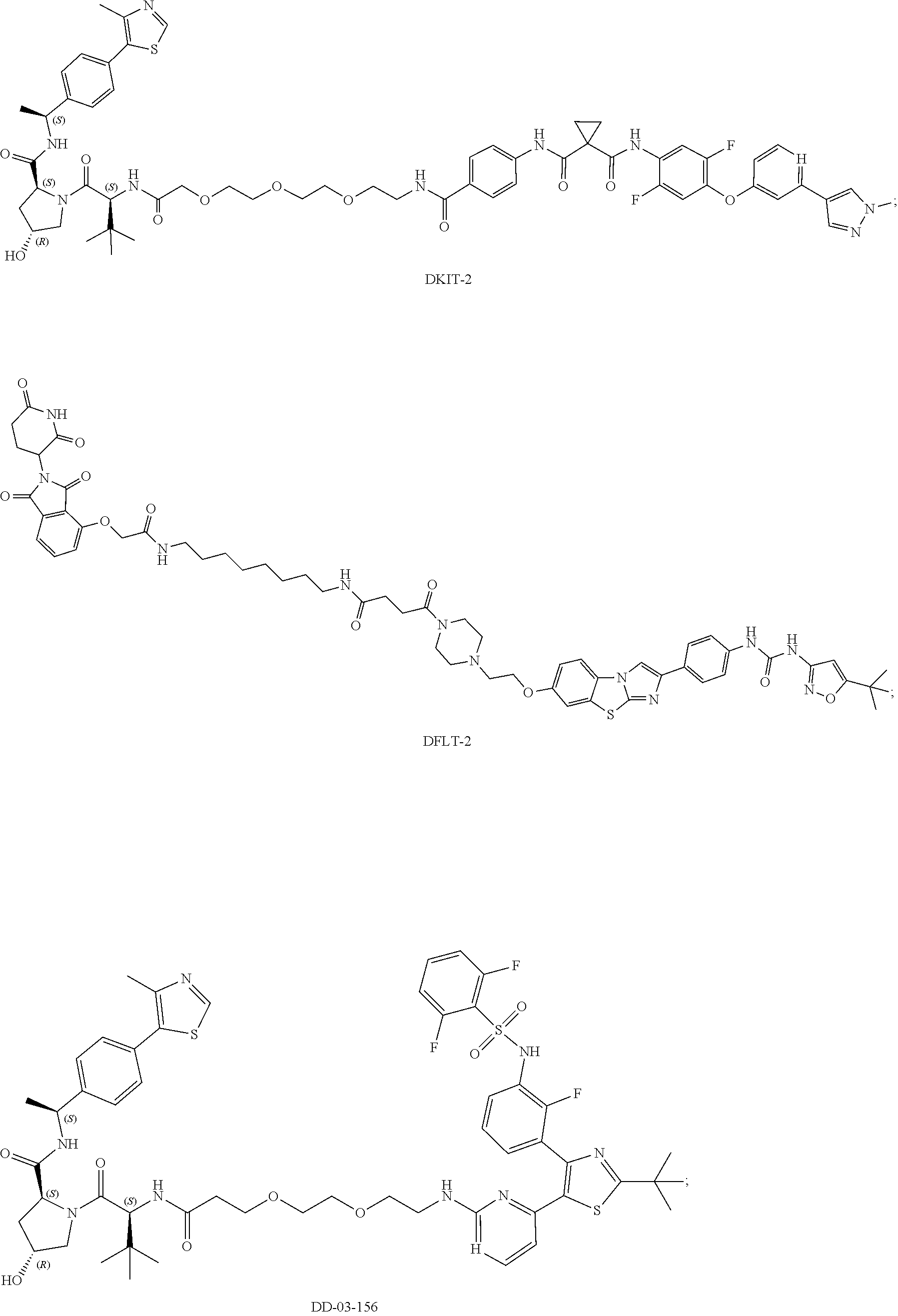 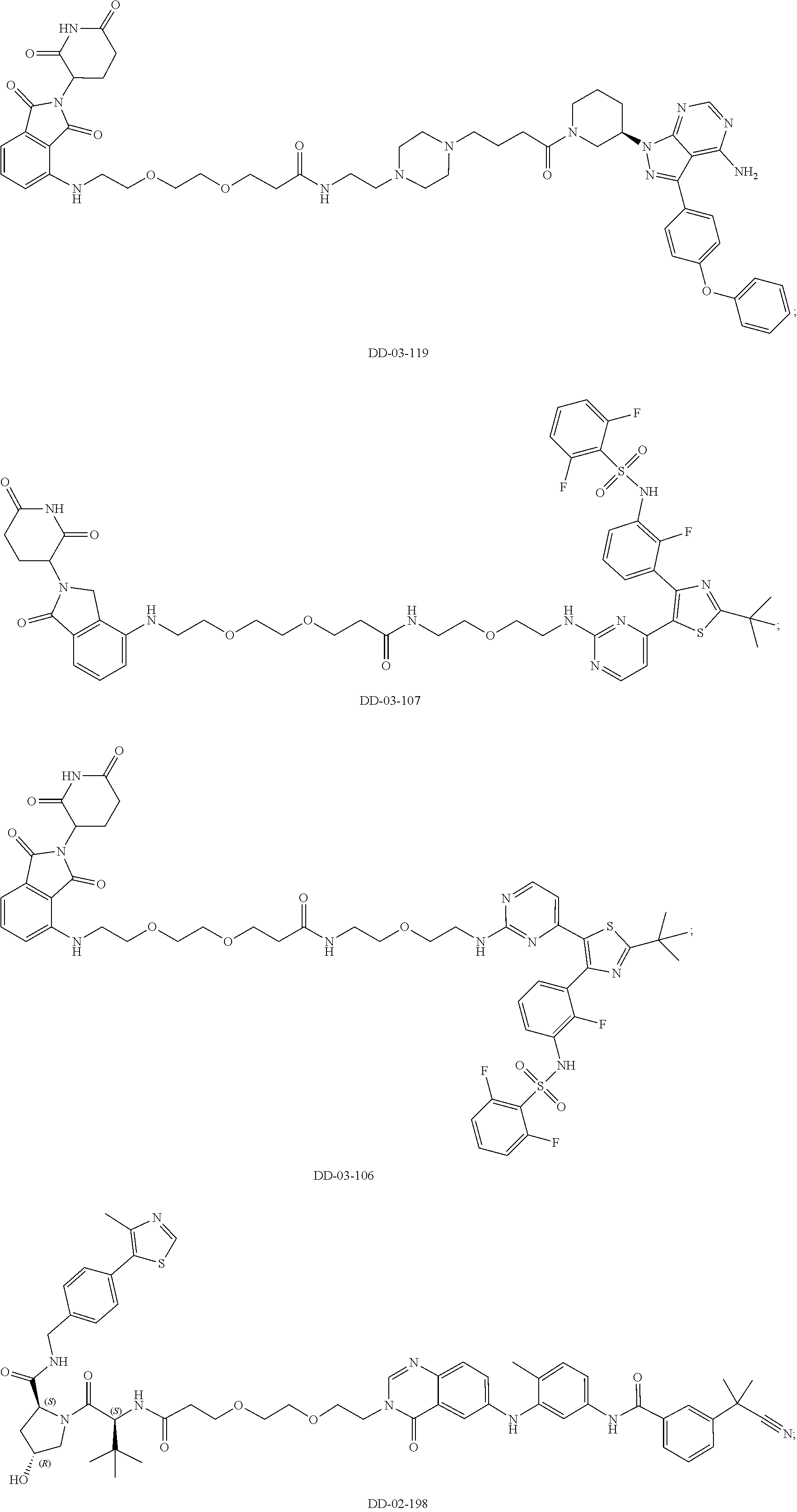 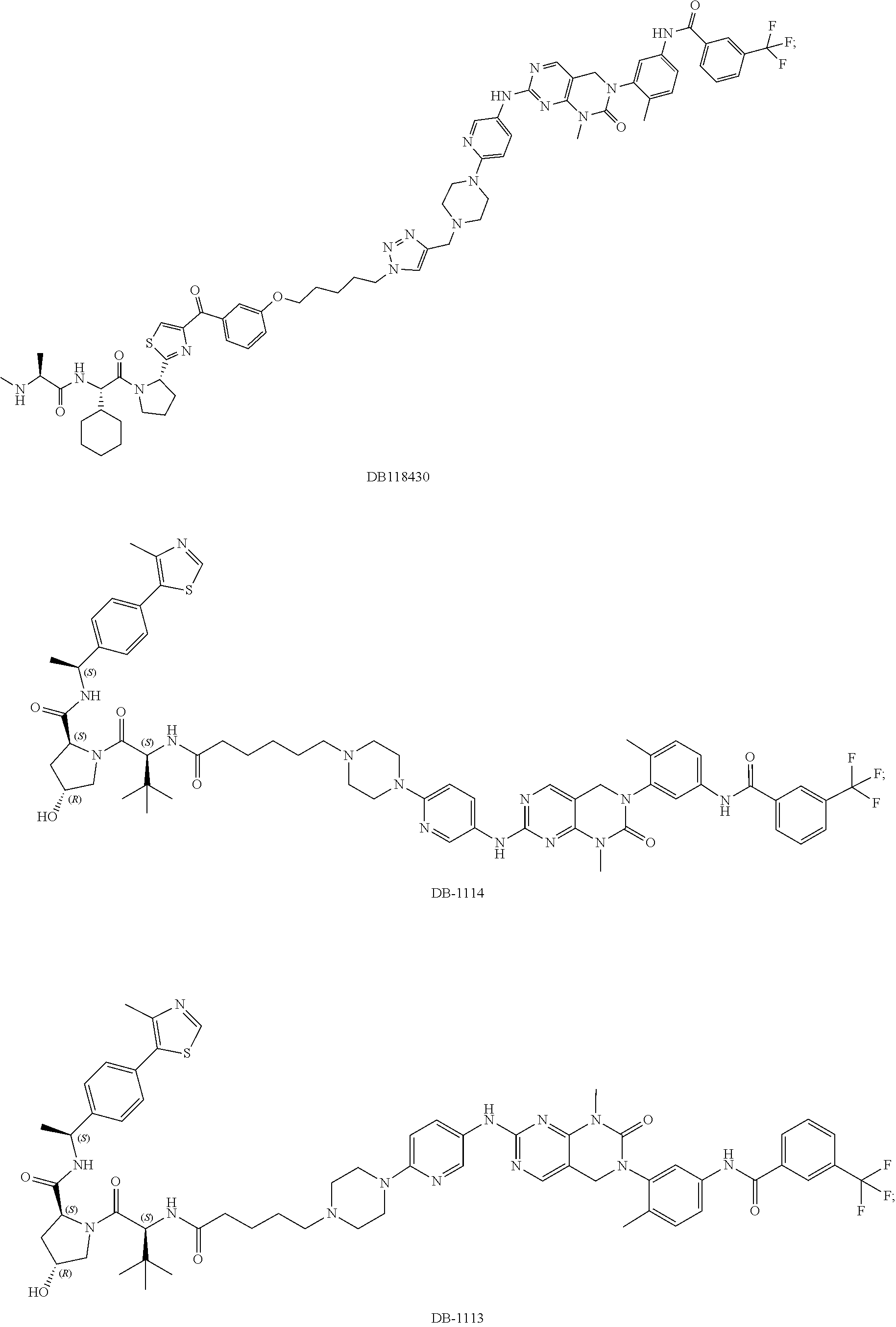 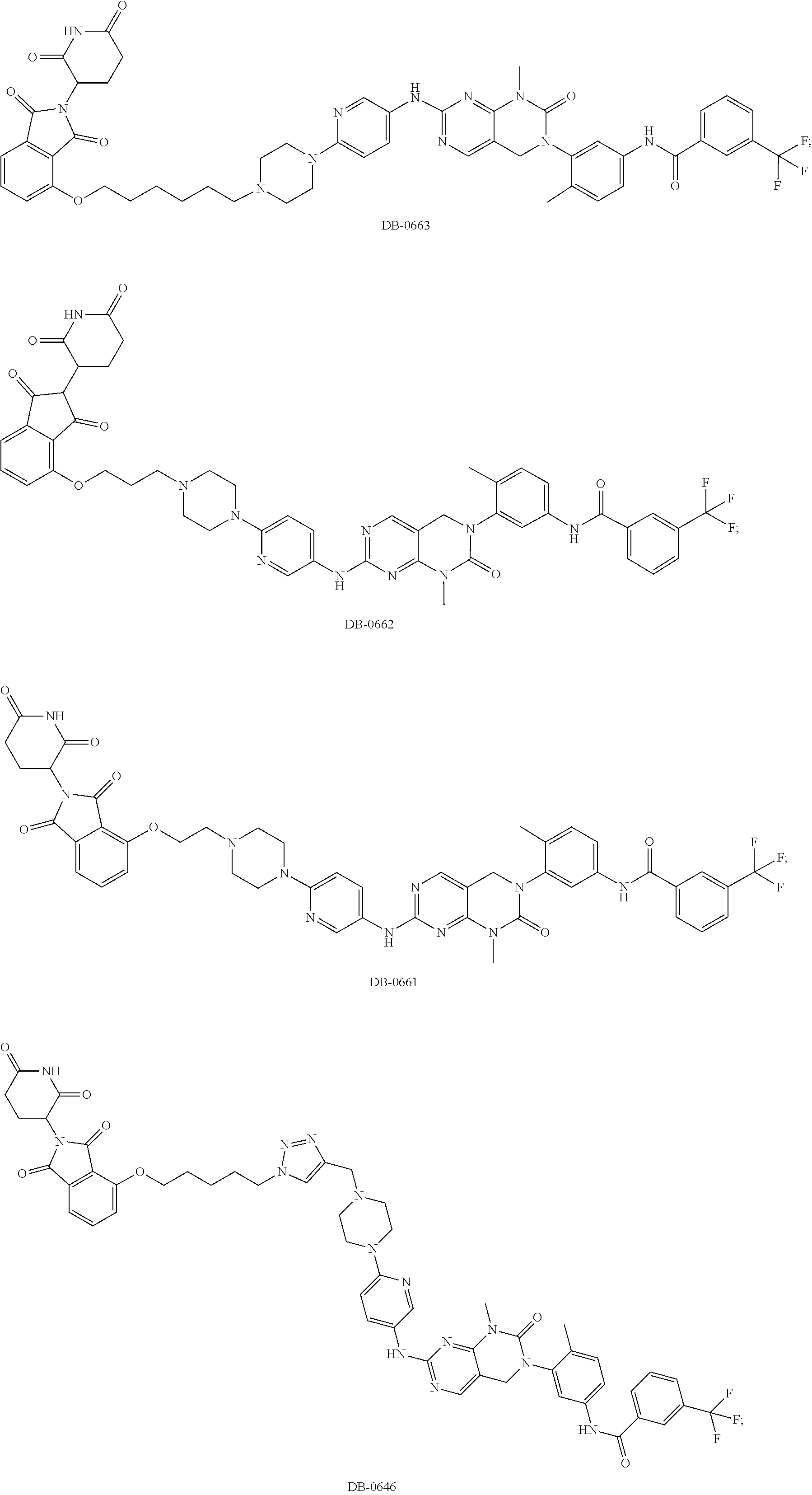 [SMILES: Cc1ccc(NC(=O)c2cccc(C(F)(F)F)c2)cc1N1Cc2cnc(Nc3ccc(N4CCN(CCCCCCOc5cccc6c5C(=O)N(C5CCC(=O)NC5=O)C6=O)CC4)nc3)nc2N(C)C1=O ; Cc1ccc(NC(=O)c2cccc(C(F)(F)F)c2)cc1N1Cc2cnc(Nc3ccc(N4CCN(CCCOc5cccc6c5C(=O)C(C5CCC(=O)NC5=O)C6=O)CC4)nc3)nc2N(C)C1=O ; Cc1ccc(NC(=O)c2cccc(C(F)(F)F)c2)cc1N1Cc2cnc(Nc3ccc(N4CCN(CCOc5cccc6c5C(=O)N(C5CCC(=O)NC5=O)C6=O)CC4)nc3)nc2N(C)C1=O ; Cc1ccc(NC(=O)c2cccc(C(F)(F)F)c2)cc1N1Cc2cnc(Nc3ccc(N4CCN(Cc5cn(CCCCCOc6cccc7c6C(=O)N(C6CCC(=O)NC6=O)C7=O)nn5)CC4)nc3)nc2N(C)C1=O] 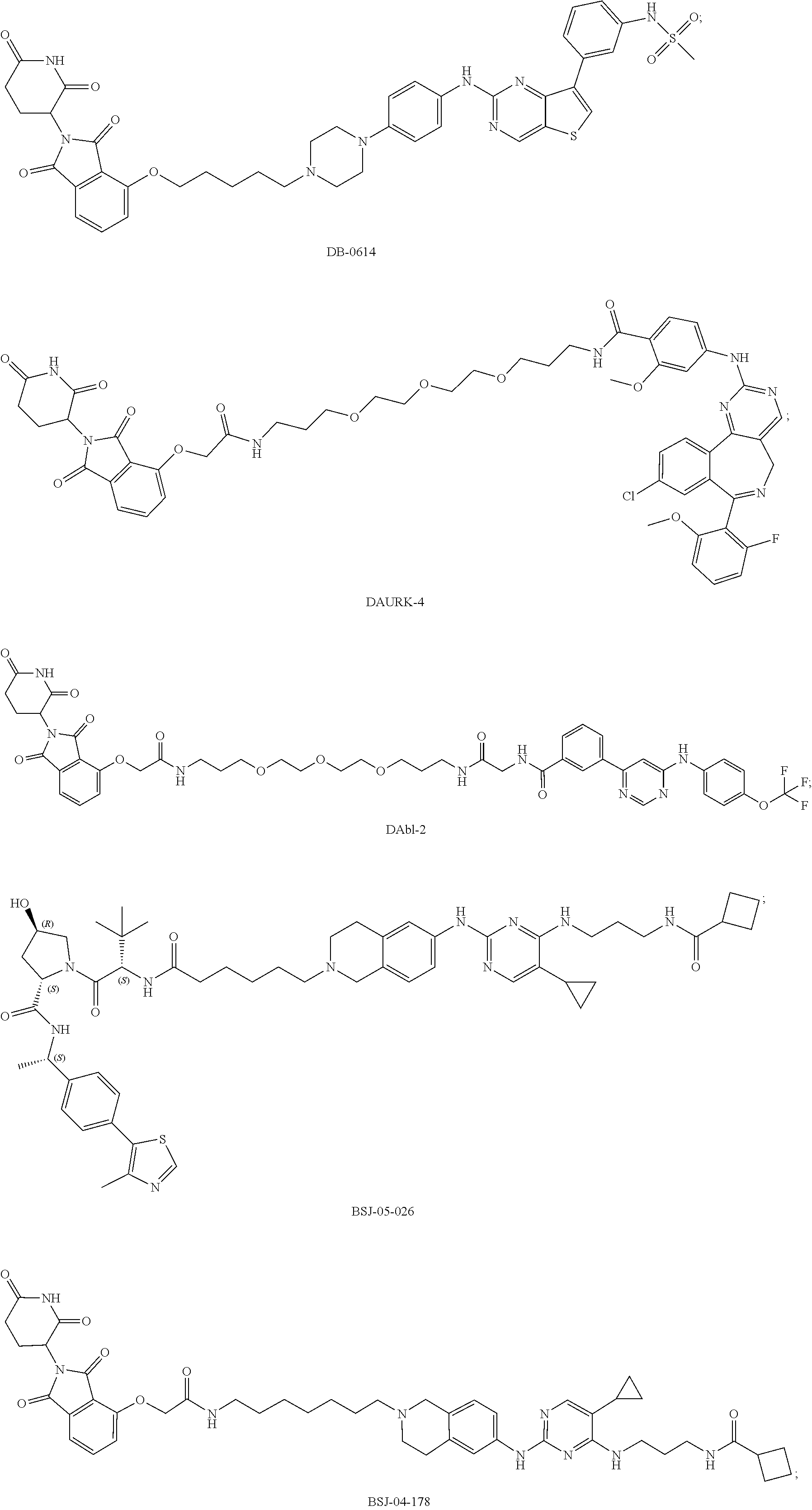 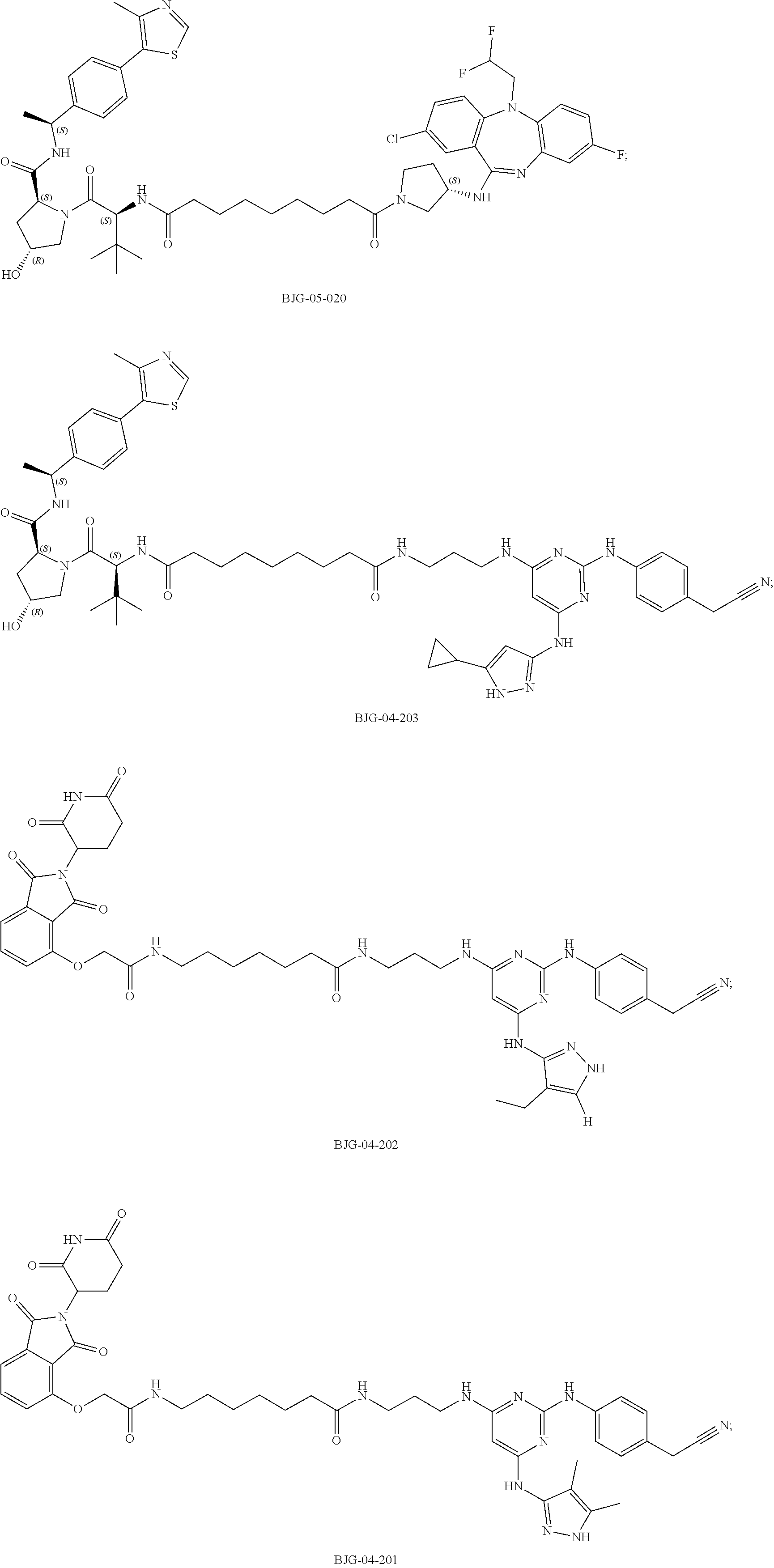 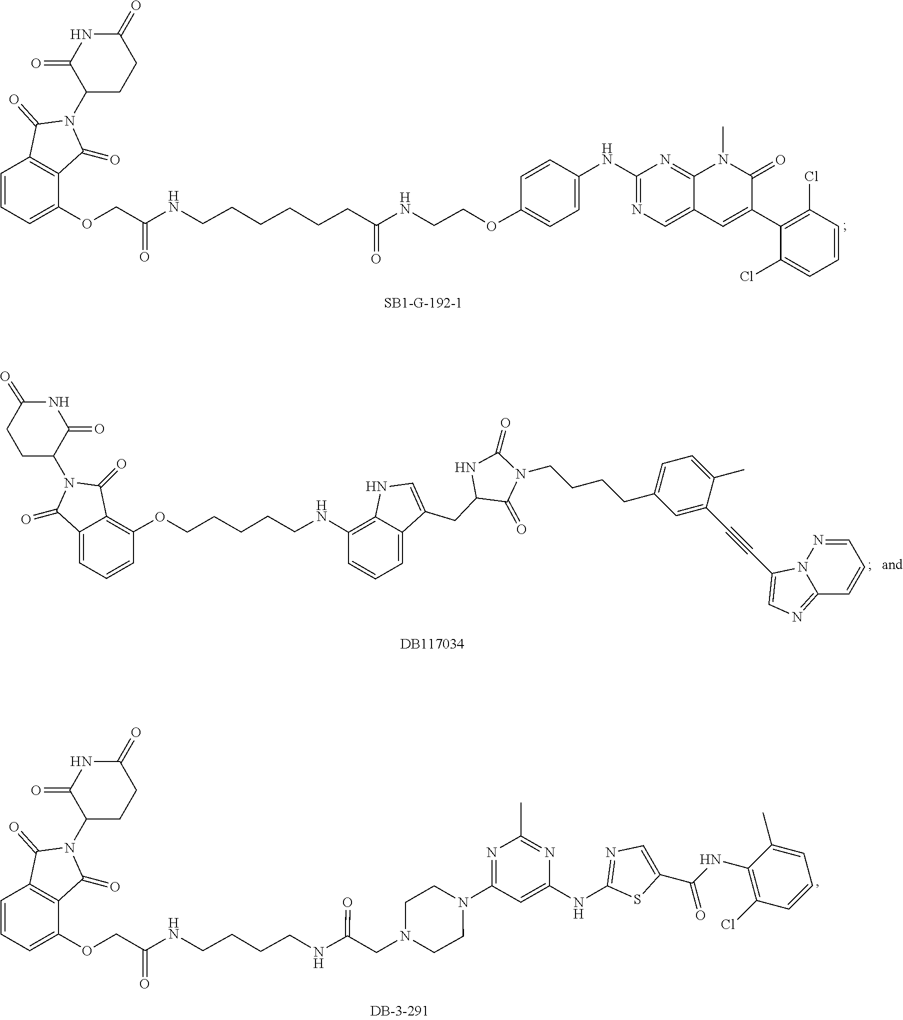 [SMILES: Cn1c(=O)c(-c2c(Cl)cccc2Cl)cc2cnc(Nc3ccc(OCCNC(=O)CCCCCCNC(=O)COc4cccc5c4C(=O)N(C4CCC(=O)NC4=O)C5=O)cc3)nc21 ; Cc1ccc(CCCCN2C(=O)NC(Cc3c[nH]c4c(NCCCCCOc5cccc6c5C(=O)N(C5CCC(=O)NC5=O)C6=O)cccc34)C2=O)cc1C#Cc1cnc2cccnn12 ; Cc1nc(Nc2ncc(C(=O)Nc3c(C)cccc3Cl)s2)cc(N2CCN(CC(=O)NCCCCNC(=O)COc3cccc4c3C(=O)N(C3CCC(=O)NC3=O)C4=O)CC2)n1]
and pharmaceutically acceptable salts or stereoisomers thereof.

2. The bifunctional compound of claim 1, which degrades at least one of
AP2-associated protein kinase 1 (AAK1), ABL proto-oncogene (ABL)1, ABL2, Serine/Threonine kinase (AKT)2, AKT3, Aurora kinase (AURK)4, AURKA, AURKB, branched chain ketoacid dehydrogenase kinase (BCKDK), B-lymphoid tyrosine kinase (BLK), BMP-2-inducible protein kinase (BMP2K), Bone morphogenetic protein receptor type-1A (BMPR1A), mitotic checkpoint serine/threonine-protein kinase BUB 1 (BUB1), BUB1B, calcium/calmodulin-dependent protein kinase kinase 1 (CAMKK1), cell division cycle 7 (CDC7), cyclin-dependent kinase (CDK)1, CDK10, CDK11A, CDK11B, CDK12, CDK13, CDK14, CDK16, CDK17, CDK18, CDK2, CDK3, CDK4, CDK5, CDK6, CDK7, CDK9, Checkpgint kinase 1(CHEK1), citron Rho-interacting kinase (CIT), CDC Like Kinase 1 (CLK1), coenzyme Q8 (COQ8)A, C008B, Tyrosine-protein kinase CSK (CSK), casein kinase 1 (CSNK1)A1, CSNK1D, CSNK1E, death-associated protein kinase 1 (DAPK1), discoidin domain-containing receptor 2 (DDR2), eukaryotic translation initiation factor 2-alpha kinase (EIF2AK)2, EIF2AK4, ephrin type-A receptor (EPHA)1, EPHA2, EPHA3, ephrin type-B receptor (EPHB)2, EPHB3, EPHB4, EPHB6, endoplasmic reticulum to nucleus signaling 1 (ERN1), tyrosine-protein kinase Fer (FER), fibroblast growth factor receptor 1 (FGFR1), fibroblast growth factor receptor 2 (FGR2), proto-oncogene tyrosine-protein kinase Fyn (FYN), cyclin G-associated kinase (GAK), glycogen synthase kinase 3 (GSK3)A, GSK3B, homeodomain-interacting protein kinase 1 (HIPK1), interleukin-1 receptor-associated kinase (IRAK)1, IRAK4, tyrosine-protein kinase ITK/TSK (ITK), large tumor suppressor kinase 1 (LATS1), lymphocyte cell-specific protein-tyrosine kinase (LCK), LIM domain kinase (LIMK)1, LIMK2, leucine-rich repeat kinase 2 (LRRK2), tyrosine-protein kinase Lyn (LYN), dual specificity mitogen-activated protein kinase kinase 5 (MAP2K5), mitogen-activated protein kinase kinase kinase (MAP3K)1, MAP3K11, MAP3K12, MAP3K20, MAP3K21, MAP3K7, mitogen-activated protein kinase kinase kinase kinase (MAP4K)1, MAP4K2, MAP4K3, MAP4K5, mitogen-activated protein kinase (MAPK)11, MAPK12, MAPK14, MAPK6, MAPK7, MAPK8, MAPK9, mitogen-activated protein kinase-activated protein kinase (MAPKAPK)2, MAPKAPK3, MAPKAPK5, microtubule affinity regulating kinase (MARK)2, MARK3, MARK4, microtubule-associated serine/threonine-protein kinase 3 (MAST3), maternal embryonic leucine zipper kinase (MELK), misshapen like kinase 1 (MINK1), MAP kinase-interacting serine/threonine-protein kinase 2 (MKNK2), never in mitosis A-related kinase (NEK)2, NEK9, nemo like kinase (NLK), NUAK family SNF1-like kinase 1 (NUAK1), serine/threonine-protein kinase PAK 4 (PAK4), serine/threonine-protein kinase PDIK1L (PDIK1L), 3-phosphoinositide-dependent protein kinase (PDK)1, PDK2, PDK3, phosphatidylinositol 4,5-bisphosphate 3-kinase catalytic subunit gamma isoform (PIK3CG), serine/threonine-protein kinase pim-2 (PIM2), membrane-associated tyrosine- and threonine-specific cdc2-inhibitory kinase (PKMYT1), serine/threonine-protein kinase N3 (PKN3), polo like kinase (PLK)1, PLK4, PEAK1 related, kinase-activating pseudokinase 1 (PRAG1), 5-AMP-activated protein kinase catalytic subunit alpha (PRKAA)1, PRKAA2, protein tyrosine kinase (PTK)2, PTK2B, PTK6, RIO kinase 2 (RIOK2), receptor-interacting serine/threonine-protein kinase (RIPK)1, RIPK2, ribosomal protein S6 kinase 2 alpha (RPS6KA)1, RPS6KA3, RPS6KA4, RPS6KA6, ribosomal protein S6 kinase beta 1 (RPS6KB1), ribosomal protein S6 kinase beta C1 (RPS6KC1), SH3 domain binding kinase 1 (SBK1), serum/glucocorticoid-regulated kinase 3 (SGK3), salt inducible kinase (SIK)2, SIK3, SIKA2, sucrose nonfermenting 1-related kinase (SNRK), proto-oncogene tyrosine-protein kinase Src (SRC), serine/threonine-protein kinase (STK)10, STK17A, STK17B, STK32C, STK33, STK35, STK38, STK4, STK40, thousand and one amino-acid kinase (TAOK)2, TAOK3, tyrosine-protein kinase Tec (TEC), dual specificity testis-specific protein kinase 2 (TESK2), transforming growth factor beta receptor 1 (TGFBR1), tyrosine kinase non receptor (TNK)1, TNK2, Tribbles homolog 3 (TRIB3), transient receptor potential cation channel subfamily M member 7 (TRPM7), dual specificity protein kinase TTK (TTK), non-receptor tyrosine-protein kinase (TYK2) TYK2, U2AF homology motif kinase 1 (UHMK1), unc-51 like autophagy activating kinase (ULK)1, ULK3, WEE1 G2 checkpoint kinase (WEE1), and YES proto-oncogene 1 (YES1).

3.-49. (canceled)

50. A pharmaceutical composition, comprising a therapeutically effective amount of the bifunctional compound or pharmaceutically acceptable salt or stereoisomer thereof of claim 1, and a pharmaceutically acceptable carrier.

51. A method of treating a disease or disorder that is characterized or mediated by aberrant activity of at least one kinase, comprising administering to a subject in need thereof a therapeutically effective amount of the compound or pharmaceutically acceptable salt or stereoisomer thereof of claim 1, wherein the disease or disorder is cancer, a neurodegenerative disease, an autoimmune disease, an infectious disease, or inflammatory disorder.

52.-57. (canceled)

58. A method of using the bifunctional compound of claim 1 as a tool for rapidly interrogating targeted protein degradation of a plurality of kinases.

59. A method for identifying a degradable kinase comprising:
assembling a kinase-targeting degrader library comprising a plurality of kinase-targeting scaffolds;
prescreening candidate degrader compounds for cellular permeability in a relevant E3-ligase target engagement assay;
selecting a cell permeable degrader for further characterization of degradation targets;
treating a cell with the selected cell permeable degrader;
employing whole cell multiplexed quantitative proteomics to measure changes in abundance of the proteome in response to treatment with the degrader relative to DMSO; and
analyzing the generated datasets to calculate kinase degradation frequency across the library, as a measure of target tractability.

60. The method of claim 59, wherein the degradation targets are further characterized using unbiased mass-spectrometry-based global proteomics analysis, based on chemical diversity and ranking in cellular ligase engagement assays relative to close analogs.

61. The method of claim 59, wherein the relevant E3-ligase target engagement assay comprises a cereblon (CRBN) or Von Hippel-Lindau tumor suppressor (VHL) target engagement assay.

62. The method of claim 59, wherein the cell is a mammalian cell.

63. The method of claim 63, wherein the mammalian cell is a human cell.

64. The method of claim 62, wherein the cell is a myeloid cell, lymphoid cell, neural cell, epithelial cell, endothelial cell, stem or progenitor cell, hepatocyte, myoblast, osteoblast, osteoclast, lymphocyte, keratinocyte, melanocyte, mesothelial cell, germ cell, muscle cell, fibroblast, transformed cell, or cancer cell.

65. The method of claim 64, wherein the cell is a HEK293T, MOLT-4, Mino, MM1.S, OVCAR-8, KATO III, or KELLY cell.

66. The method of claim 59, wherein the cell is treated with a cell permeable degrader for 1 h, 2 h, 3 h, 4 h, 5 h, 6 h, 7 h, or 8 h.

67. The method of claim 66, wherein the cell is treated with a cell permeable degrader for 5 h.

68. The method of claim 59, wherein the cell is treated with 0.1-10 μM cell permeable degrader.

69. The method of claim 68, wherein the cell is treated with 0.1-5 μM cell permeable degrader.

70. The method of claim 68, wherein the cell is treated with 1 μM cell permeable degrader.

71. The method of claim 59, the abundance fold change cutoff is set at −1.25, and P-value <0.01.

72. The method of claim 59, which is used for rapidly identifying optimal kinase:scaffold pairs.
